(19) United States
(12) Patent Application Publication
(10) Pub. No.: US 2025/0002913 A1
(43) Pub. Date: Jan. 2, 2025

(54) NUCLEIC ACIDS CONTAINING ABASIC NUCLEOSIDES

(71) Applicant: E-THERAPEUTICS PLC, London (GB)

(72) Inventors: Amy MCCARTHY, London (GB); Graham CRAGGS, London (GB); James LONGDEN, London (GB); Ines DE SANTIAGO, London (GB); Duncan BROWN, London (GB); Ahmad ALI MORTAZAVI, London (GB); Viviana MANNELLA, London (GB); Muthusamy JAYARAMAN, Walpole, MA (US)

(21) Appl. No.: 18/698,235

(22) PCT Filed: Jul. 27, 2022
(86) PCT No.: PCT/US2022/074223

§ 371 Date: Apr. 3, 2024

Related U.S. Application Data
(60) Provisional application No. 63/262,316, filed on Oct. 8, 2021.
(60) Provisional application No. 63/271,684, filed on Oct. 25, 2021.

(30) Foreign Application Priority Data
Jan. 28, 2022 (WO) PCT/EP2022/052070

(51) Int. Cl. C12N 15/113

(52) U.S. Cl. CPC C12N 15/113; C12N 15/1137; C12N 2310/14; C12N 2310/313; C12N 2310/321; C12N 2310/322; C12N 2310/332; C12N 2310/351

(57) ABSTRACT

The present invention relates to nucleic acid molecules for use in the treatment or prevention of disease.

### CROSS-REFERENCE TO RELATED APPLICATIONS

This application claims priority to U.S. Provisional Patent Application No. 63/262,316, filed Oct. 8, 2021, and U.S. Provisional Patent Application No. 63/271,684, filed Oct. 25, 2021, and International Application No. PCT/EP2022/052070, filed Jan. 28, 2022, the content of each of which are incorporated herein by reference in their entirety.

### FIELD

The present invention provides novel oligonucleoside compounds, which are nucleic acid compounds, suitable for therapeutic use. Additionally, the present invention provides methods of making these compounds, as well as methods of using such compounds for the treatment of various diseases and conditions.

### BACKGROUND OF THE INVENTION

Oligonucleotide/oligonucleoside compounds have important therapeutic applications in medicine. Oligonucleotides/oligonucleosides can be used to silence genes that are responsible for a particular disease. Gene-silencing prevents formation of a protein by inhibiting translation. Importantly, gene-silencing agents are a promising alternative to traditional small, organic compounds that inhibit the function of the protein linked to the disease. siRNA, antisense RNA, and micro-RNA are oligonucleotides/oligonucleosides that prevent the formation of proteins by gene-silencing.

A number of modified siRNA compounds in particular have been developed in the last two decades for diagnostic and therapeutic purposes, including siRNA/RNAi therapeutic agents for the treatment of various diseases including central-nervous-system diseases, inflammatory diseases, metabolic disorders, oncology, infectious diseases, and ocular diseases.

The present invention relates to such oligonucleoside compounds, which are nucleic acid compounds, for use in the treatment and/or prevention of disease.

### STATEMENTS OF INVENTION

A nucleic acid, optionally an RNA, for inhibiting expression of a target gene in a cell, comprising at least one duplex region that comprises at least a portion of a first strand and at least a portion of a second strand that is at least partially complementary to the first strand, wherein said first strand is at least partially complementary to at least a portion of RNA transcribed from said target gene to be inhibited, wherein the second strand comprises one or more abasic nucleosides in a terminal region of the second strand, and wherein said abasic nucleoside(s) is/are connected to an adjacent nucleoside through a reversed internucleoside linkage.

A conjugate for inhibiting expression of a target gene in a cell, said conjugate comprising a nucleic acid portion and one or more ligand moieties, said nucleic acid portion comprising a nucleic acid as disclosed herein.

A pharmaceutical composition comprising a nucleic acid as disclosed herein or a conjugate as disclosed herein and a physiologically acceptable excipient.

### FIGURES

FIG. 1 shows analysis of hsC5 mRNA expression levels in a total of 45 human-derived cancer cell lysates and lysates of primary human hepatocytes (PHHs). mRNA expression levels are shown in relative light units [RLUs].

FIG. 2 shows analysis of hsHAO1 mRNA expression levels in a total of 45 human-derived cancer cell lysates and lysates of primary human hepatocytes (PHHs). mRNA expression levels are shown in relative light units [RLUs].

FIG. 3 shows analysis of hsTTR mRNA expression levels in a total of 45 human-derived cancer cell lysates and lysates of primary human hepatocytes (PHHs). mRNA expression levels are shown in relative light units [RLUs].

FIGS. 4A-B shows the results from the dose-response analysis of hsTTR targeting GalNAc-siRNAs in HepG2 cells in Example 1.

FIGS. 5A-B shows the results from the dose-response analysis of hsC5 targeting GalNAc-siRNAs in HepG2 cells in Example 1.

FIG. 6 shows the analysis of hsTTR (top), hsC5 (middle) and hsHAO1 (bottom) mRNA expression levels in all three batches of primary human hepatocytes BHuf16087 (left), CHF2101 (middle) and CyHuf19009 (right) each after 0 h, 24 h, 48 h and 72 h in culture. mRNA expression levels are shown in relative light units [RLUs].

FIG. 7 shows the analysis of hsGAPDH (top) and hsAHSA1 (bottom) mRNA expression levels in all three batches of primary human hepatocytes BHuf16087 (left), CHF2101 (middle) and CyHuf19009 (right) each after 0 h, 24 h, 48 h and 72 h in culture. mRNA expression levels are shown in relative light units [RLUs].

FIGS. 8A-B shows the results from the dose-response analysis of hsHAO1 targeting GalNAc-siRNAs in PHHs in Example 1.

FIGS. 9A-B shows the results from the dose-response analysis of hsC5 targeting GalNAc-siRNAs in PHHs in Example 1.

FIGS. 10A-B shows the results from the dose-response analysis of hsTTR targeting GalNAc-siRNAs in PHHs in Example 1.

FIGS. 11 A-B shows the results from the dose-response analysis of hsTTR targeting GalNAc-siRNAs in HepG2 cells in Example 3.

FIGS. 12 A-B shows the results from the dose-response analysis of hsC5 targeting GalNAc-siRNAs in HepG2 cells in Example 3.

FIGS. 13 A-B shows the results from the dose-response analysis of hsHAO1 targeting GalNAc-siRNAs in PHHs in Example 3.

FIGS. 14 A-B shows the results from the dose-response analysis of hsC5 targeting GalNAc-siRNAs in PHHs in Example 3.

FIGS. 15 A-B shows the results from the dose-response analysis of hsTTR targeting GalNAc-siRNAs in PHHs in Example 3.

FIG. 16 Single dose mouse pharmacology of ETX005. HAO1 mRNA expression is shown relative to the saline control group. Each point represents the mean and standard deviation of 3 mice.

FIG. 17 Single dose mouse pharmacology of ETX005. Serum glycolate concentration is shown. Each point represents the mean and standard deviation of 3 mice, except for baseline glycolate concentration (day 0) which was derived from a group of 5 mice.

FIG. 18 Single dose mouse pharmacology of ETX006. HAO1 mRNA expression is shown relative to the saline control group. Each point represents the mean and standard deviation of 3 mice.

FIG. 19 Single dose mouse pharmacology of ETX006. Serum glycolate concentration is shown. Each point represents the mean and standard deviation of 3 mice, except for baseline glycolate concentration (day 0) which was derived from a group of 5 mice.

FIG. 20 Single dose mouse pharmacology of ETX014. C5 mRNA expression is shown relative to the saline control group. Each point represents the mean and standard deviation of 3 mice.

FIG. 21 Single dose mouse pharmacology of ETX0014. Serum C5 concentration is shown relative to the saline control group. Each point represents the mean and standard deviation of 3 mice.

FIG. 22 Single dose mouse pharmacology of ETX015. C5 mRNA expression is shown relative to the saline control group. Each point represents the mean and standard deviation of 3 mice.

FIG. 23 Single dose mouse pharmacology of ETX0015. Serum C5 concentration is shown relative to the saline control group. Each point represents the mean and standard deviation of 3 mice.

FIG. 24 Single dose NHP pharmacology of ETX023. Serum TTR concentration is shown relative to day 1 of the study. Each point represents the mean and standard deviation of 3 animals.

FIG. 25 Single dose NHP pharmacology of ETX024. Serum TTR concentration is shown relative to day 1 of the study. Each point represents the mean and standard deviation of 3 animals.

FIG. 26 Single dose NHP pharmacology of ETX019. Serum TTR concentration is shown relative to day 1 of the study and also pre-dose. Each point represents the mean and standard deviation of 3 animals. Time points up to 84 days are shown.

FIG. 27 Single dose NHP pharmacology of ETX020. Serum TTR concentration is shown relative to day 1 of the study and also pre-dose. Each point represents the mean and standard deviation of 3 animals. Time points up to 84 days are shown.

FIG. 28a Single dose NHP pharmacology of ETX023. Serum TTR concentration is shown relative to day 1 of the study and also pre-dose. Each point represents the mean and standard deviation of 3 animals. Time points up to 84 days are shown.

FIG. 28b Sustained suppression of TTR gene expression in the liver after a single 1 mg/kg dose of ETX023. TTR mRNA is shown relative to baseline levels measured pre-dose. Each point represents the mean and standard deviation of 3 animals. Time points up to 84 days are shown.

FIG. 28c Body weight of animals dosed with a single 1 mg/kg dose of ETX023. Each point represents the mean and standard deviation of 3 animals. Time points up to 84 days are shown.

FIG. 28d ALT concentration in serum from animals treated with a single 1 mg/kg dose of ETX023. Each point represents the mean and standard deviation of 3 animals. The dotted lines show the range of values considered normal for this species (Park et al. 2016 Reference values of clinical pathology parameter in cynomolgus monkeys used in preclinical studies. Lab Anim Res 32:79-86.) Time points up to 84 days are shown.

FIG. 28e AST concentration in serum from animals treated with a single 1 mg/kg dose of ETX023. Each point represents the mean and standard deviation of 3 animals. The dotted lines show the range of values considered normal for this species (Park et al. 2016 Reference values of clinical pathology parameter in cynomolgus monkeys used in preclinical studies. Lab Anim Res 32:79-86. Time points up to 84 days are shown.

FIG. 29a Single dose NHP pharmacology of ETX024. Serum TTR concentration is shown relative to day 1 of the study and also pre-dose. Each point represents the mean and standard deviation of 3 animals. Time points up to 84 days are shown.

FIG. 29b Sustained suppression of TTR gene expression in the liver after a single 1 mg/kg dose of ETX024. TTR mRNA is shown relative to baseline levels measured pre-dose. Each point represents the mean and standard deviation of 3 animals. Time points up to 84 days are shown.

FIG. 29c Body weight of animals dosed with a single 1 mg/kg dose of ETX024. Each point represents the mean and standard deviation of 3 animals. Time points up to 84 days are shown.

FIG. 29d ALT concentration in serum from animals treated with a single 1 mg/kg dose of ETX024. Each point represents the mean and standard deviation of 3 animals. The dotted lines show the range of values considered normal for this species (Park et al. 2016 Reference values of clinical pathology parameter in cynomolgus monkeys used in preclinical studies. Lab Anim Res 32:79-86.) Time points up to 84 days are shown.

FIG. 29e AST concentration in serum from animals treated with a single 1 mg/kg dose of ETX024. Each point represents the mean and standard deviation of 3 animals. The dotted lines show the range of values considered normal for this species (Park et al. 2016 Reference values of clinical pathology parameter in cynomolgus monkeys used in preclinical studies. Lab Anim Res 32:79-86. Time points up to 84 days are shown.

FIG. 30 Linker and ligand portion of ETX005, 014, 023

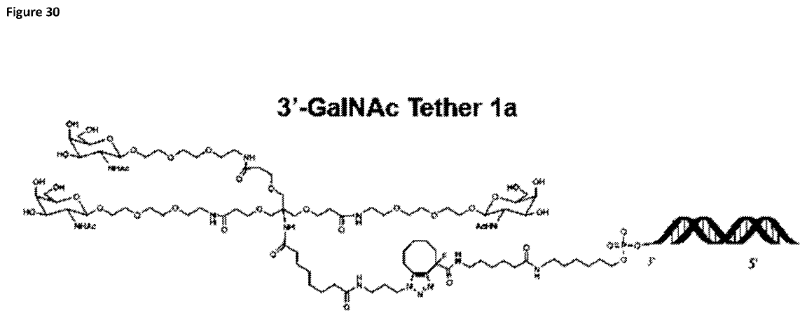

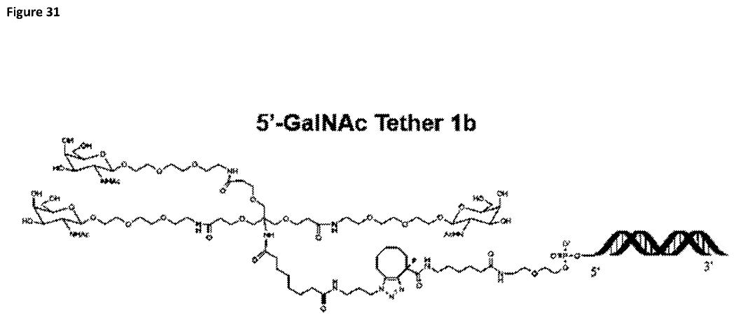

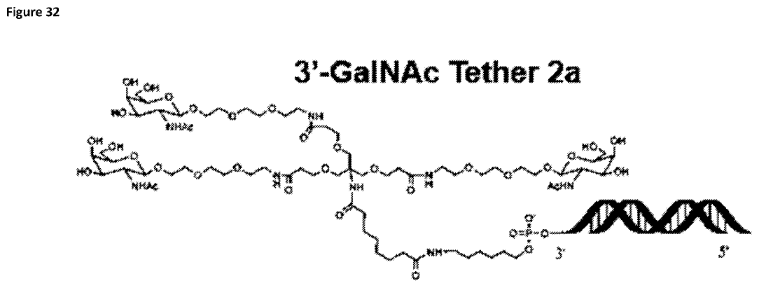

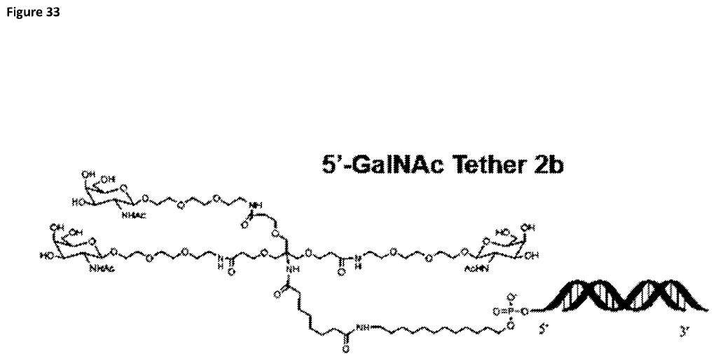

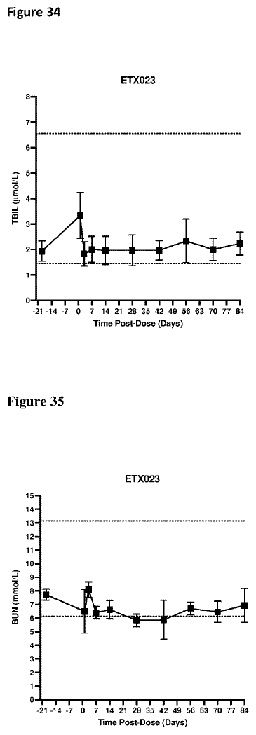

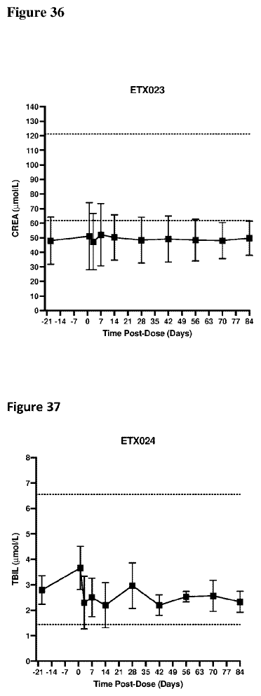

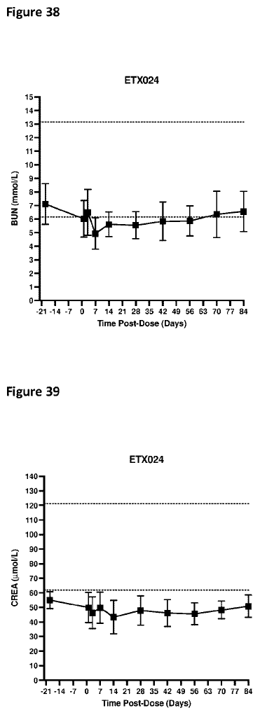

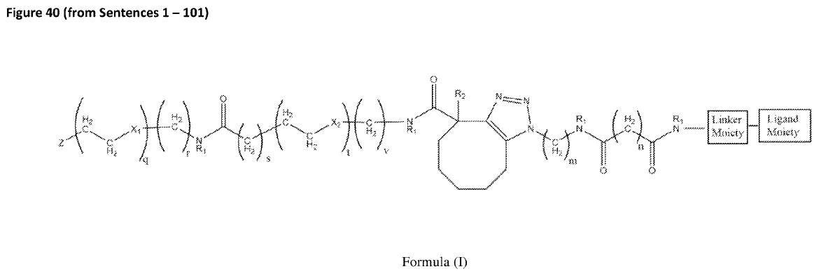

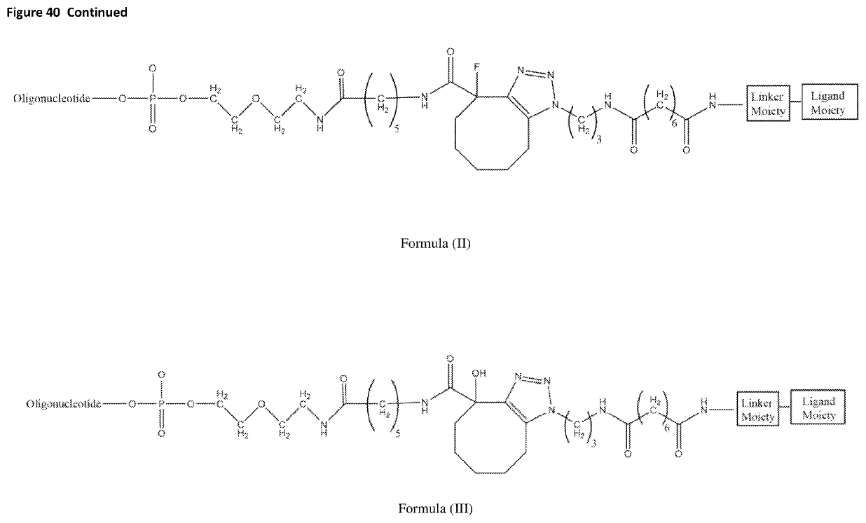

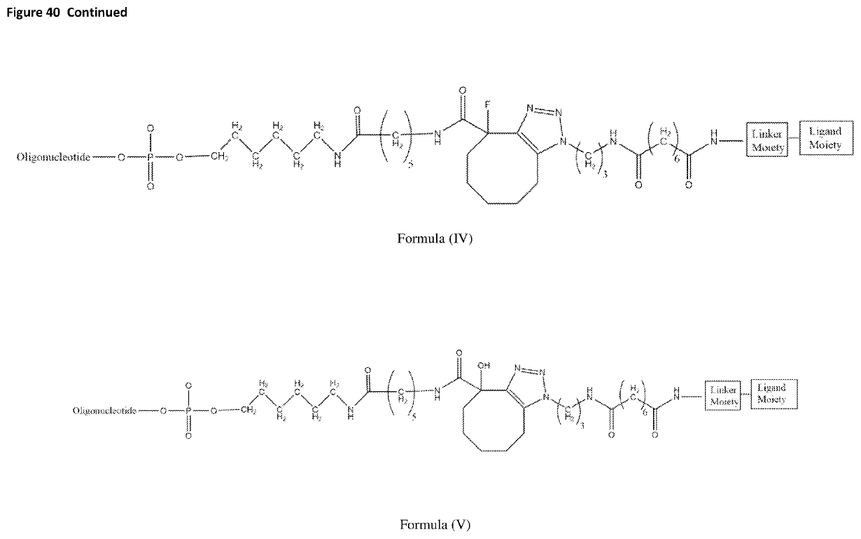

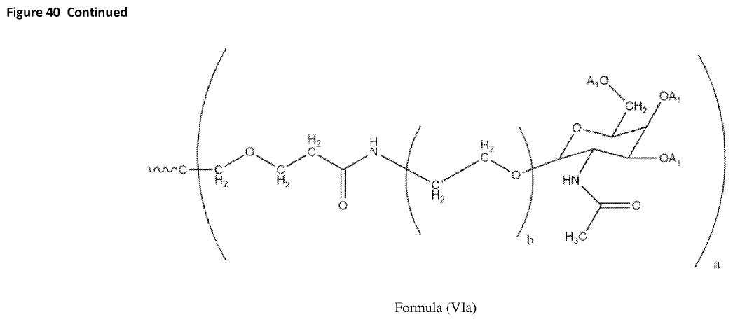

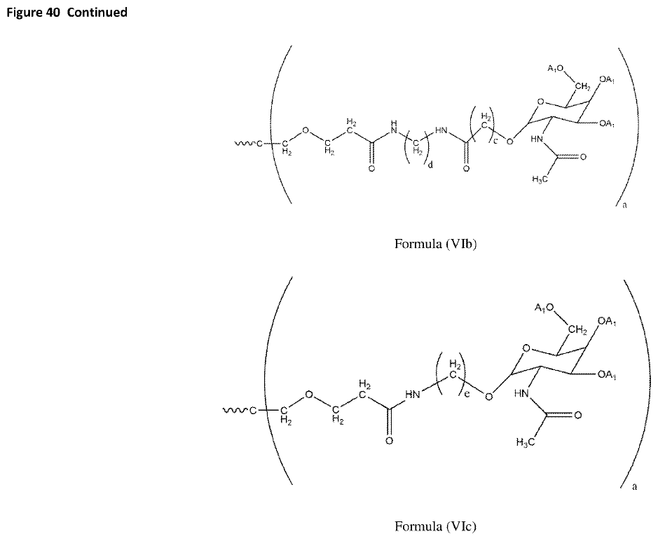

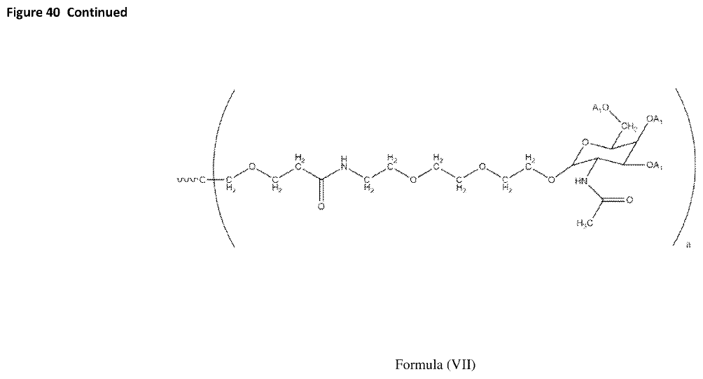

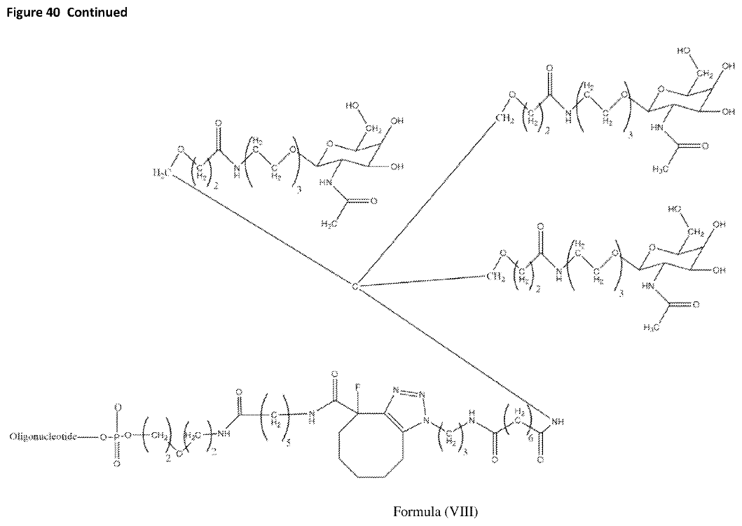

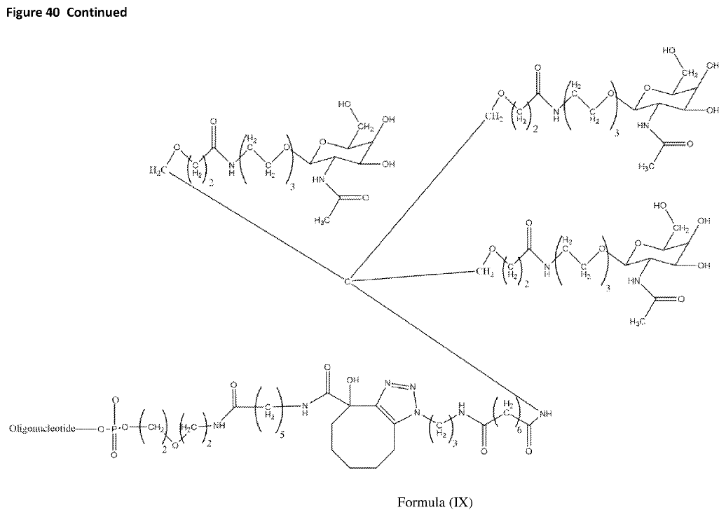

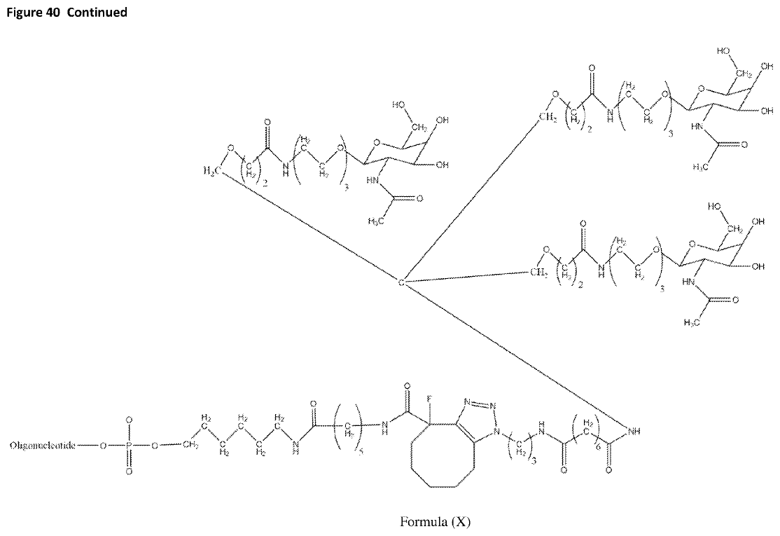

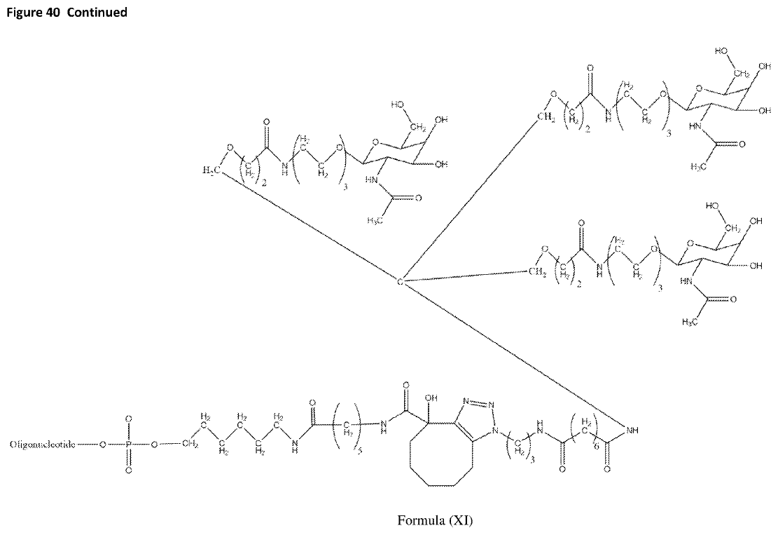

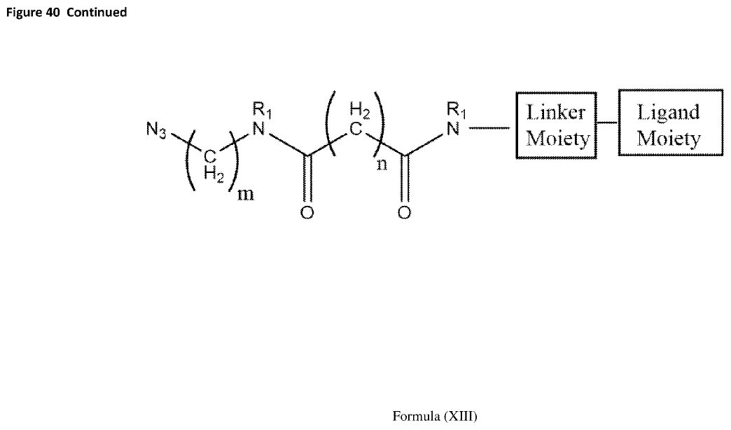

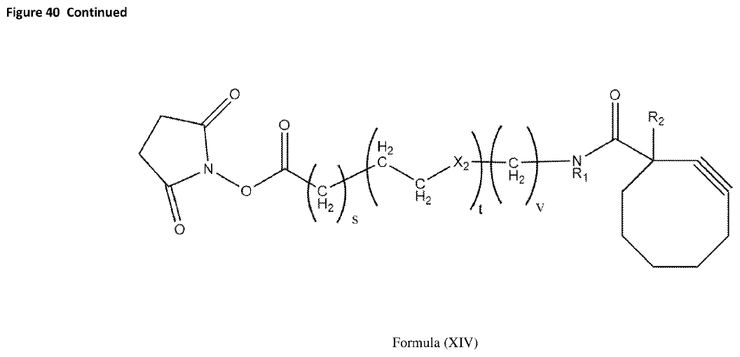

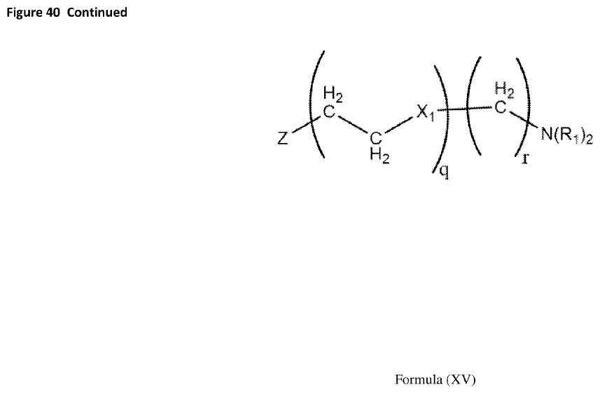

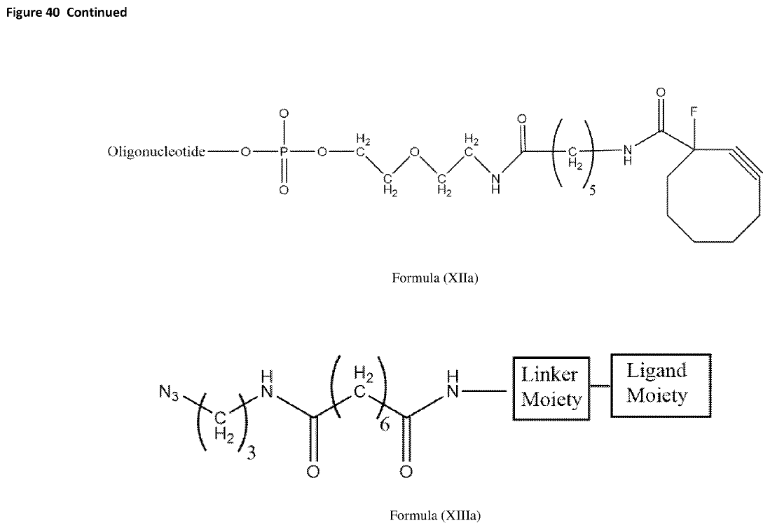

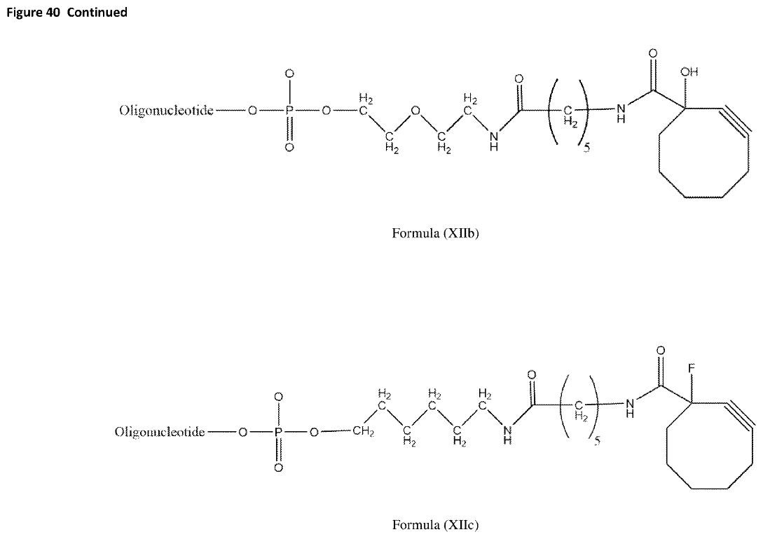

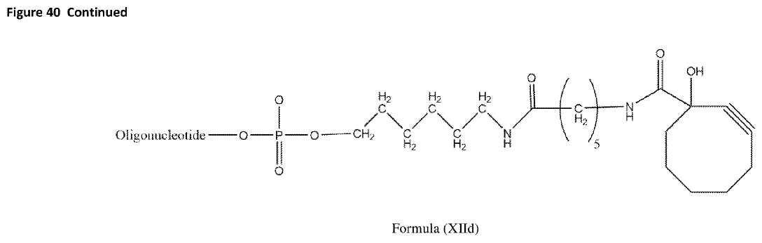

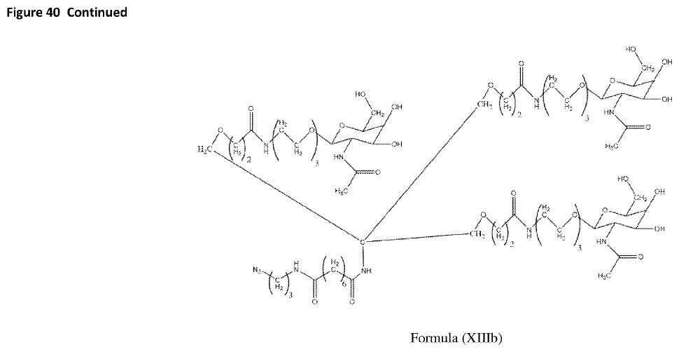

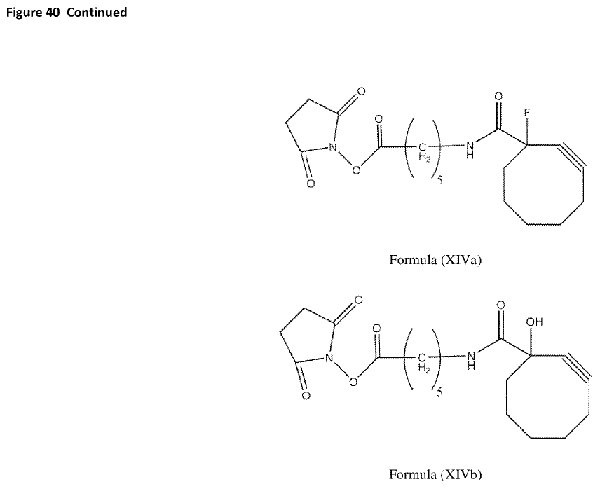

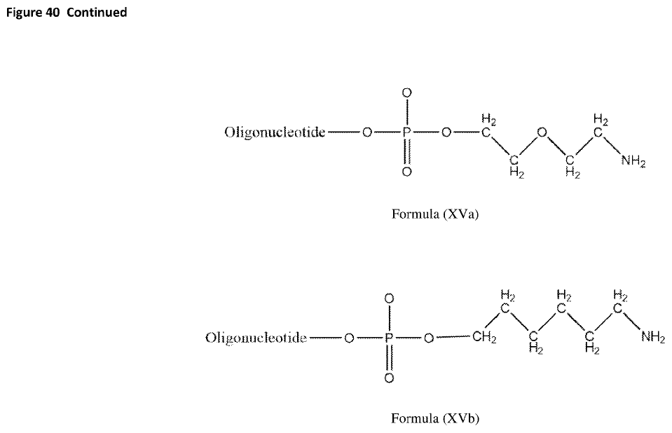

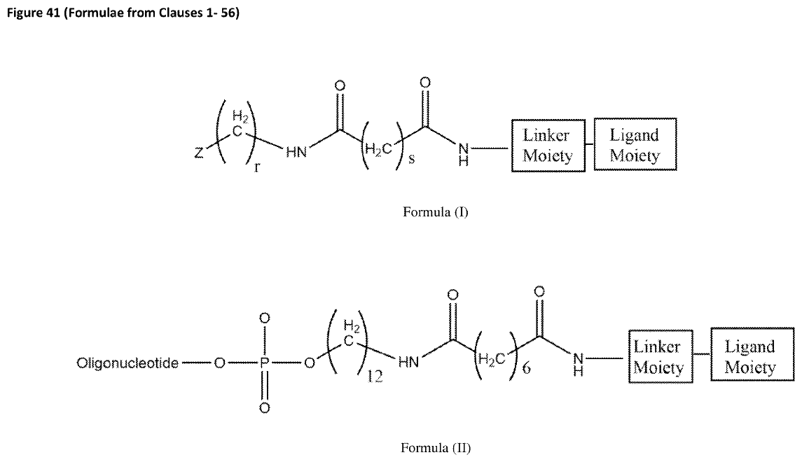

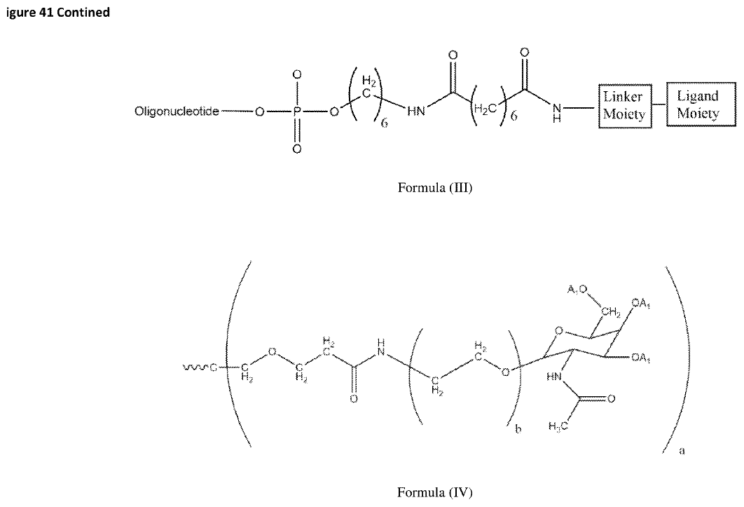

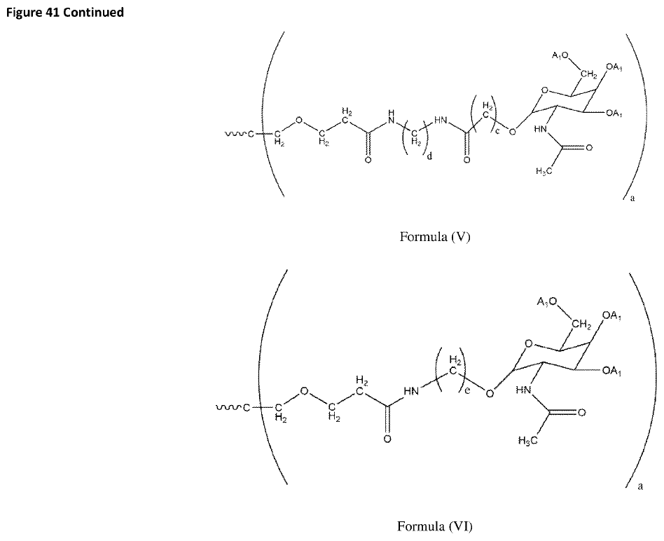

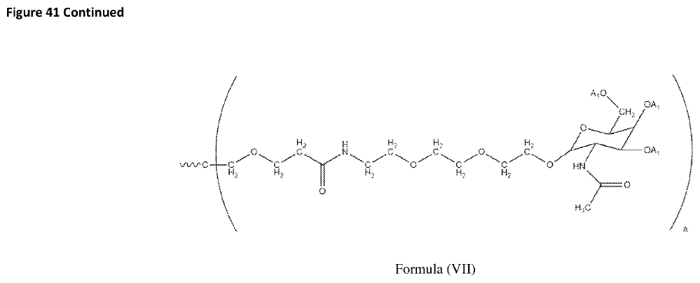

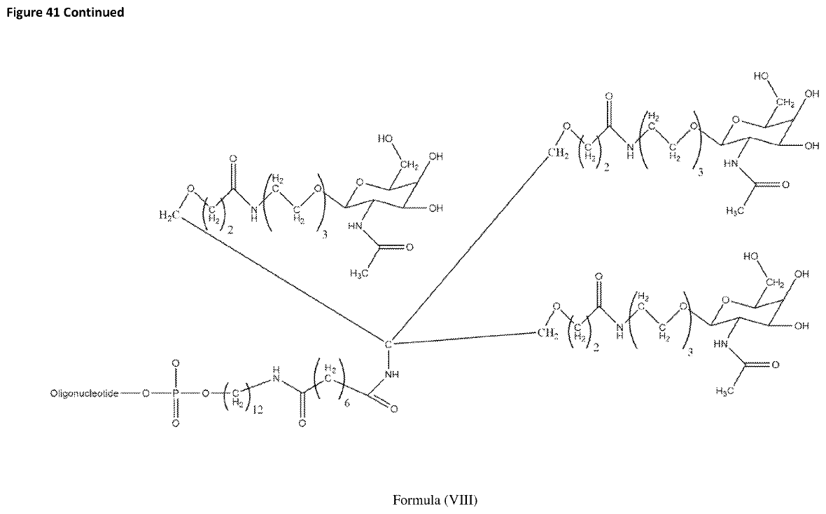

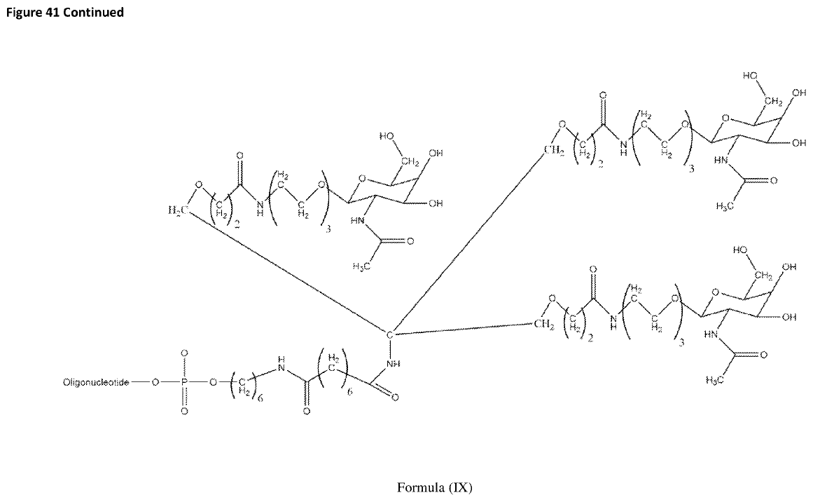

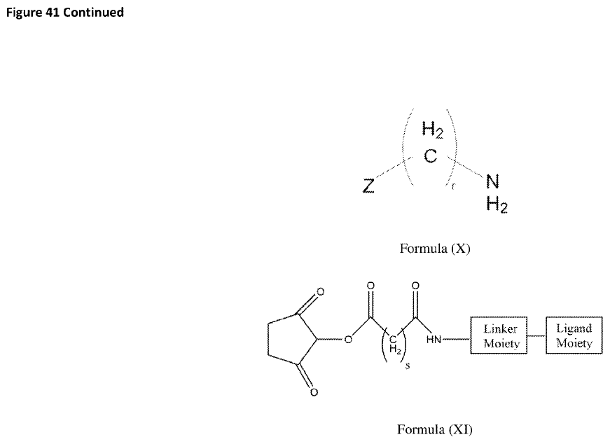

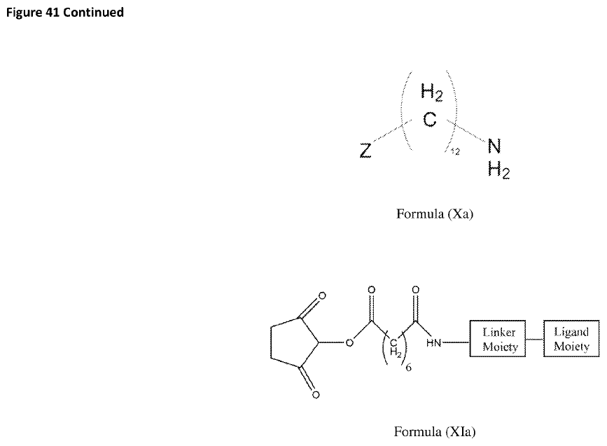

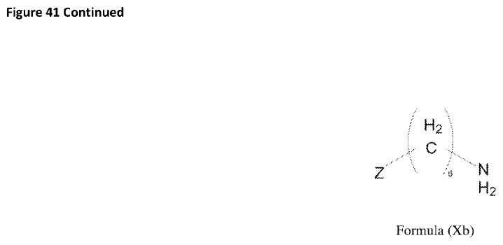

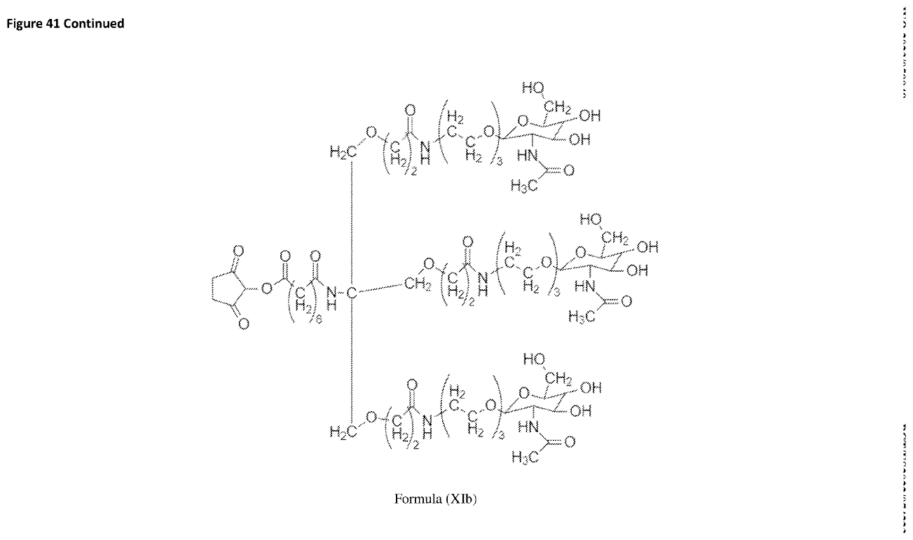

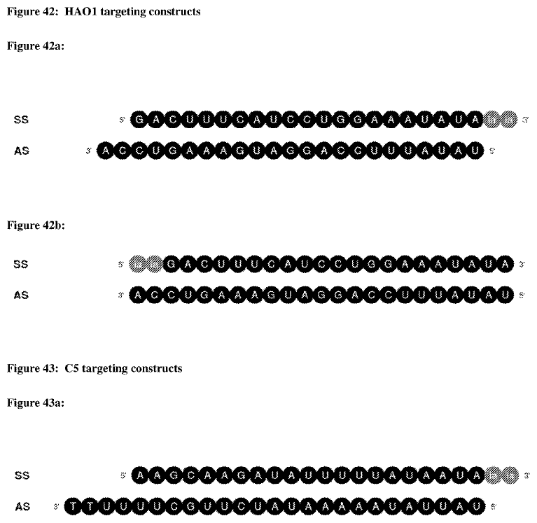

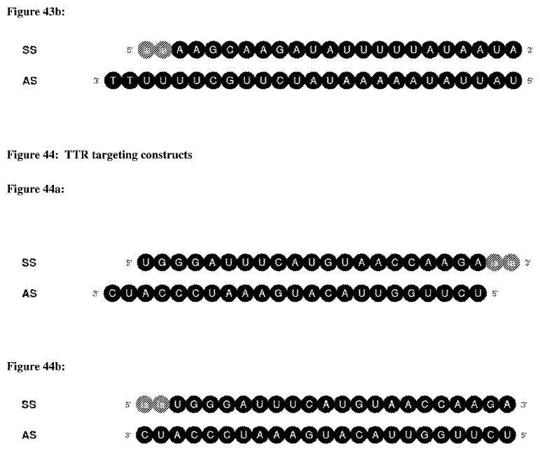

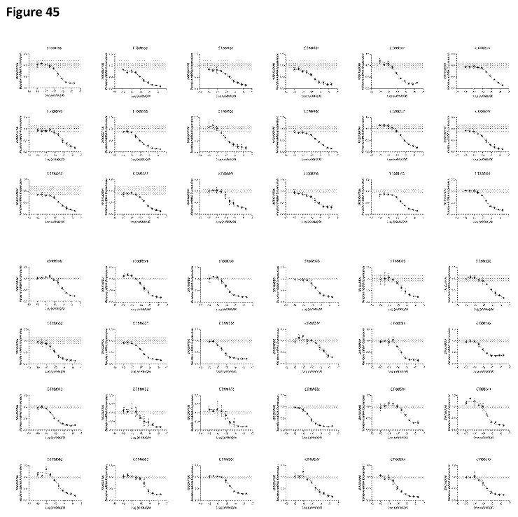

It should also be understood that where appropriate while ETX005 as a product includes molecules based on the linker and ligand portions as specifically depicted in FIG. 30 attached to an oligonucleoside moiety as also depicted herein, this ETX005 product may alternatively further comprise, or consist essentially of, molecules wherein the linker and ligand portions are essentially as depicted in FIG. 30 attached to an oligonucleoside moiety but having the F substituent as shown in FIG. 30 on the cyclo-octyl ring replaced by a substituent occurring as a result of hydrolytic displacement, such as an OH substituent. In this way, (a) ETX005 can consist essentially of molecules having linker and ligand portions specifically as depicted in FIG. 30, with a F substituent on the cyclo-octyl ring; or (b) ETX005 can consist essentially of molecules having linker and ligand portions essentially as depicted in FIG. 30 but having the F substituent as shown in FIG. 30 on the cyclo-octyl ring replaced by a substituent occurring as a result of hydrolytic displacement, such as an OH substituent, or (c) ETX005 can comprise a mixture of molecules as defined in (a) and/or (b).

It should also be understood that where appropriate while ETX014 as a product includes molecules based on the linker and ligand portions as specifically depicted in FIG. 30 attached to an oligonucleoside moiety as also depicted herein, this ETX014 product may alternatively further comprise, or consist essentially of, molecules wherein the linker and ligand portions are essentially as depicted in FIG. 30 attached to an oligonucleoside moiety but having the F substituent as shown in FIG. 30 on the cyclo-octyl ring replaced by a substituent occurring as a result of hydrolytic displacement, such as an OH substituent. In this way, (a) ETX014 can consist essentially of molecules having linker and ligand portions specifically as depicted in FIG. 30, with a F substituent on the cyclo-octyl ring; or (b) ETX014 can consist essentially of molecules having linker and ligand portions essentially as depicted in FIG. 30 but having the F substituent as shown in FIG. 30 on the cyclo-octyl ring replaced by a substituent occurring as a result of hydrolytic displacement, such as an OH substituent, or (c) ETX014 can comprise a mixture of molecules as defined in (a) and/or (b).

It should also be understood that where appropriate while ETX023 as a product includes molecules based on the linker and ligand portions as specifically depicted in FIG. 30 attached to an oligonucleoside moiety as also depicted herein, this ETX023 product may alternatively further comprise, or consist essentially of, molecules wherein the linker and ligand portions are essentially as depicted in FIG. 30 attached to an oligonucleoside moiety but having the F substituent as shown in FIG. 30 on the cyclo-octyl ring replaced by a substituent occurring as a result of hydrolytic displacement, such as an OH substituent. In this way, (a) ETX023 can consist essentially of molecules having linker and ligand portions specifically as depicted in FIG. 30, with a F substituent on the cyclo-octyl ring; or (b) ETX023 can consist essentially of molecules having linker and ligand portions essentially as depicted in FIG. 30 but having the F substituent as shown in FIG. 30 on the cyclo-octyl ring replaced by a substituent occurring as a result of hydrolytic displacement, such as an OH substituent, or (c) ETX023 can comprise a mixture of molecules as defined in (a) and/or (b).

FIG. 31 Linker and ligand portion of ETX001, 010 and 019

It should also be understood that where appropriate while ETX001 as a product includes molecules based on the linker and ligand portions as specifically depicted in FIG. 31 attached to an oligonucleoside moiety as also depicted herein, this ETX001 product may alternatively further comprise, or consist essentially of, molecules wherein the linker and ligand portions are essentially as depicted in FIG. 31 attached to an oligonucleoside moiety but having the F substituent as shown in FIG. 31 on the cyclo-octyl ring replaced by a substituent occurring as a result of hydrolytic displacement, such as an OH substituent. In this way, (a) ETX001 can consist essentially of molecules having linker and ligand portions specifically as depicted in FIG. 31, with a F substituent on the cyclo-octyl ring; or (b) ETX001 can consist essentially of molecules having linker and ligand portions essentially as depicted in FIG. 31 but having the F substituent as shown in FIG. 31 on the cyclo-octyl ring replaced by a substituent occurring as a result of hydrolytic displacement, such as an OH substituent, or (c) ETX001 can comprise a mixture of molecules as defined in (a) and/or (b).

It should also be understood that where appropriate while ETX010 as a product includes molecules based on the linker and ligand portions as specifically depicted in FIG. 31 attached to an oligonucleoside moiety as also depicted herein, this ETX010 product may alternatively further comprise, or consist essentially of, molecules wherein the linker and ligand portions are essentially as depicted in FIG. 31 attached to an oligonucleoside moiety but having the F substituent as shown in FIG. 31 on the cyclo-octyl ring replaced by a substituent occurring as a result of hydrolytic displacement, such as an OH substituent. In this way, (a) ETX010 can consist essentially of molecules having linker and ligand portions specifically as depicted in FIG. 31, with a F substituent on the cyclo-octyl ring; or (b) ETX010 can consist essentially of molecules having linker and ligand portions essentially as depicted in FIG. 31 but having the F substituent as shown in FIG. 31 on the cyclo-octyl ring replaced by a substituent occurring as a result of hydrolytic displacement, such as an OH substituent, or (c) ETX010 can comprise a mixture of molecules as defined in (a) and/or (b).

It should also be understood that where appropriate while ETX019 as a product includes molecules based on the linker and ligand portions as specifically depicted in FIG. 31 attached to an oligonucleoside moiety as also depicted herein, this ETX019 product may alternatively further comprise, or consist essentially of, molecules wherein the linker and ligand portions are essentially as depicted in FIG. 31 attached to an oligonucleoside moiety but having the F substituent as shown in FIG. 31 on the cyclo-octyl ring replaced by a substituent occurring as a result of hydrolytic displacement, such as an OH substituent. In this way, (a) ETX019 can consist essentially of molecules having linker and ligand portions specifically as depicted in FIG. 31, with a F substituent on the cyclo-octyl ring; or (b) ETX019 can consist essentially of molecules having linker and ligand portions essentially as depicted in FIG. 31 but having the F substituent as shown in FIG. 31 on the cyclo-octyl ring replaced by a substituent occurring as a result of hydrolytic displacement, such as an OH substituent, or (c) ETX019 can comprise a mixture of molecules as defined in (a) and/or (b).

FIG. 32 Linker and ligand portion of ETX006, 015 and 024

FIG. 33 Linker and ligand portion of ETX002, 011 and 020.

FIG. 34 Total bilirubin concentration in serum from animals treated with a single 1 mg/kg dose of ETX023. Each point represents the mean and standard deviation of 3 animals. The dotted lines show the range of values considered normal for this species (Park et al. 2016 Reference values of clinical pathology parameter in cynomolgus monkeys used in preclinical studies. Lab Anim Res 32:79-86.)

FIG. 35. Blood urea nitrogen (BUN) concentration from animals treated with a single 1 mg/kg dose of ETX023. Each point represents the mean and standard deviation of 3 animals. The dotted lines show the range of values considered normal for this species (Park et al. 2016 Reference values of clinical pathology parameter in cynomolgus monkeys used in preclinical studies. Lab Anim Res 32:79-86.)

FIG. 36 Creatinine (CREA) concentration from animals treated with a single 1 mg/kg dose of ETX023. Each point represents the mean and standard deviation of 3 animals. The dotted lines show the range of values considered normal for this species (Park et al. 2016 Reference values of clinical pathology parameter in cynomolgus monkeys used in preclinical studies. Lab Anim Res 32:79-86.)

FIG. 37 Total bilirubin concentration in serum from animals treated with a single 1 mg/kg dose of ETX024. Each point represents the mean and standard deviation of 3 animals. The shaded are shows the range of values considered normal at the facility used for the study. The dotted lines show values considered normal for this species (Park et al. 2016 Reference values of clinical pathology parameter in cynomolgus monkeys used in preclinical studies. Lab Anim Res 32:79-86.)

FIG. 38 Blood urea nitrogen (BUN) concentration from animals treated with a single 1 mg/kg dose of ETX024. Each point represents the mean and standard deviation of 3 animals. The shaded are shows the range of values considered normal at the facility used for the study. The dotted lines show values considered normal for this species (Park et al. 2016 Reference values of clinical pathology parameter in cynomolgus monkeys used in preclinical studies. Lab Anim Res 32:79-86.)

FIG. 39 Creatinine (CREA) concentration from animals treated with a single 1 mg/kg dose of ETX024. Each point represents the mean and standard deviation of 3 animals. The shaded are shows the range of values considered normal at the facility used for the study. The dotted lines show values considered normal for this species (Park et al. 2016 Reference values of clinical pathology parameter in cynomolgus monkeys used in preclinical studies. Lab Anim Res 32:79-86.)

FIG. 40 shows the detail of the formulae described in Sentences 1-101 disclosed herein.

FIG. 41 shows the detail of formulae described in Clauses 1-56 disclosed herein

FIG. 42a shows the underlying nucleoside sequences for the sense (SS) and antisense (AS) strands of constructs ETX001, ETX002 as described herein. For both ETX001 and ETX002 a galnac linker is attached to the 5′ end region of the sense strand in use (not depicted in FIG. 42a). For ETX001 the galnac linker is attached and as shown in FIG. 31. For ETX002 the galnac linker is attached and as shown in FIG. 33.

iaia as shown at the 3′ end region of the sense strand in FIG. 42a represents (i) two abasic nucleosides provided as the penultimate and terminal nucleosides at the 3′ end region of the sense strand, (ii) wherein a 3′-3′ reversed linkage is provided between the antepenultimate nucleoside (namely A at position 21 of the sense strand, wherein position 1 is the terminal 5′ nucleoside of the sense strand, namely terminal G at the 5′end region of the sense strand) and the adjacent penultimate abasic residue of the sense strand, and (iii) the linkage between the terminal and penultimate abasic nucleosides is 5′-3′ when reading towards the 3′ end region comprising the terminal and penultimate abasic nucleosides.

For the sense strand of FIG. 42a, when reading from position 1 of the sense strand (which is the terminal 5′ nucleoside of the sense strand, namely terminal G at the 5′end region of the sense strand), then: (i) the nucleosides at positions 1 to 6, 8, and 12 to 21 have sugars that are 2′ O-methyl modified, (ii) the nucleosides at positions 7, and 9 to 11 have sugars that are 2′ F modified, (iii) the abasic nucleosides have sugars that have H at positions 1 and 2.

For the antisense strand of FIG. 42a, when reading from position 1 of the antisense strand (which is the terminal 5′ nucleoside of the antisense strand, namely terminal U at the 5′end region of the antisense strand), then: (i) the nucleosides at positions 1, 3 to 5, 7, 10 to 13, 15, 17 to 23 have sugars that are 2′ O-methyl modified, (ii) the nucleosides at positions 2, 6, 8, 9, 14, 16 have sugars that are 2′ F modified.

FIG. 42b shows the underlying nucleoside sequences for the sense (SS) and antisense (AS) strands of constructs ETX005, ETX006 as described herein. For both ETX005 and ETX006 a galnac linker is attached to the 3′ end region of the sense strand in use (not depicted in FIG. 42b). For ETX005 the galnac linker is attached and as shown in FIG. 30. For ETX006 the galnac linker is attached and as shown in FIG. 32.

iaia as shown at the 5′ end region of the sense strand in FIG. 42b represents (i) two abasic nucleosides provided as the penultimate and terminal nucleosides at the 5′ end region of the sense strand, (ii) wherein a 5′-5′ reversed linkage is provided between the antepenultimate nucleoside (namely G at position 1 of the sense strand, not including the iaia motif at the 5′ end region of the sense strand in the nucleoside position numbering on the sense strand) and the adjacent penultimate abasic residue of the sense strand, and (iii) the linkage between the terminal and penultimate abasic nucleosides is 3′-5′ when reading towards the 5′ end region comprising the terminal and penultimate abasic nucleosides.

For the sense strand of FIG. 42b, when reading from position 1 of the sense strand (which is the terminal 5′ nucleoside of the sense strand, namely terminal G at the 5′end region of the sense strand, not including the iaia motif at the 5′ end region of the sense strand in the nucleoside position numbering on the sense strand), then: (i) the nucleosides at positions 1 to 6, 8, and 12 to 21 have sugars that are 2′ O-methyl modified, (ii) the nucleosides at positions 7, and 9 to 11 have sugars that are 2′ F modified, (iii) the abasic nucleosides have sugars that have H at positions 1 and 2.

For the antisense strand of FIG. 42b, when reading from position 1 of the antisense strand (which is the terminal 5′ nucleoside of the antisense strand, namely terminal U at the 5′end region of the antisense strand), then: (i) the nucleosides at positions 1, 3 to 5, 7, 10 to 13, 15, 17 to 23 have sugars that are 2′ O-methyl modified, (ii) the nucleosides at positions 2, 6, 8, 9, 14, 16 have sugars that are 2′ F modified.

FIG. 43a shows the underlying nucleoside sequences for the sense (SS) and antisense (AS) strands of constructs ETX010, ETX011 as described herein. For both ETX010 and ETX011 a galnac linker is attached to the 5′ end region of the sense strand in use (not depicted in FIG. 43a). For ETX010 the galnac linker is attached and as shown in FIG. 31. For ETX011 the galnac linker is attached and as shown in FIG. 33.

iaia as shown at the 3′ end region of the sense strand in FIG. 43a represents (i) two abasic nucleosides provided as the penultimate and terminal nucleosides at the 3′ end region of the sense strand, (ii) wherein a 3′-3′ reversed linkage is provided between the antepenultimate nucleoside (namely A at position 21 of the sense strand, wherein position 1 is the terminal 5′ nucleoside of the sense strand, namely terminal A at the 5′end region of the sense strand) and the adjacent penultimate abasic residue of the sense strand, and (iii) the linkage between the terminal and penultimate abasic nucleosides is 5′-3′ when reading towards the 3′ end region comprising the terminal and penultimate abasic nucleosides.

For the sense strand of FIG. 43a, when reading from position 1 of the sense strand (which is the terminal 5′ nucleoside of the sense strand, namely terminal A at the 5′end region of the sense strand), then: (i) the nucleosides at positions 1, 2, 4, 6, 8, 12, 14, 15, 17, 19 to 21 have sugars that are 2′ O-methyl modified, (ii) the nucleosides at positions 3, 5, 7, 9 to 11, 13, 16, 18 have sugars that are 2′ F modified, (iii) the abasic nucleosides have sugars that have H at positions 1 and 2.

For the antisense strand of FIG. 43a, when reading from position 1 of the antisense strand (which is the terminal 5′ nucleoside of the antisense strand, namely terminal U at the 5′end region of the antisense strand), then: (i) the nucleosides at positions 1, 4, 6, 7, 9, 11 to 13, 15, 17, 19 to 23 have sugars that are 2′ O-methyl modified, (ii) the nucleosides at positions 2, 3, 5, 8, 10, 14, 16, 18 have sugars that are 2′ F modified, (iii) the penultimate and terminal T nucleosides at positions 24, 25 at the 3′ end region of the antisense strand have sugars that have H at position 2.

FIG. 43b shows the underlying nucleoside sequences for the sense (SS) and antisense (AS) strands of constructs ETX014, ETX015 as described herein. For both ETX014 and ETX015 a galnac linker is attached to the 3′ end region of the sense strand in use (not depicted in FIG. 43b). For ETX014 the galnac linker is attached and as shown in FIG. 30. For ETX015 the galnac linker is attached and as shown in FIG. 32.

iaia as shown at the 5′ end region of the sense strand in FIG. 43b represents (i) two abasic nucleosides provided as the penultimate and terminal nucleosides at the 5′ end region of the sense strand, (ii) wherein a 5′-5′ reversed linkage is provided between the antepenultimate nucleoside (namely A at position 1 of the sense strand, not including the iaia motif at the 5′ end region of the sense strand in the nucleoside position numbering on the sense strand) and the adjacent penultimate abasic residue of the sense strand, and (iii) the linkage between the terminal and penultimate abasic nucleosides is 3′-5′ when reading towards the 5′ end region comprising the terminal and penultimate abasic nucleosides.

For the sense strand of FIG. 43b, when reading from position 1 of the sense strand (which is the terminal 5′ nucleoside of the sense strand, namely terminal A at the 5′end region of the sense strand, not including the iaia motif at the 5′ end region of the sense strand in the nucleoside position numbering on the sense strand), then: (i) the nucleosides at positions 1, 2, 4, 6, 8, 12, 14, 15, 17, 19 to 21 have sugars that are 2′ O-methyl modified, (ii) the nucleosides at positions 3, 5, 7, 9 to 11, 13, 16, 18 have sugars that are 2′ F modified, (iii) the abasic nucleosides have sugars that have H at positions 1 and 2.

For the antisense strand of FIG. 43b, when reading from position 1 of the antisense strand (which is the terminal 5′ nucleoside of the antisense strand, namely terminal U at the 5′end region of the antisense strand), then: (i) the nucleosides at positions 1, 4, 6, 7, 9, 11 to 13, 15, 17, 19 to 23 have sugars that are 2′ O-methyl modified, (ii) the nucleosides at positions 2, 3, 5, 8, 10, 14, 16, 18 have sugars that are 2′ F modified, (iii) the penultimate and terminal T nucleosides at positions 24, 25 at the 3′ end region of the antisense strand have sugars that have H at position 2.

FIG. 44a shows the underlying nucleoside sequences for the sense (SS) and antisense (AS) strands of constructs ETX019, ETX020 as described herein. For both ETX019 and ETX020 a galnac linker is attached to the 5′ end region of the sense strand in use (not depicted in FIG. 44a). For ETX019 the galnac linker is attached and as shown in FIG. 31. For ETX020 the galnac linker is attached and as shown in FIG. 33.

iaia as shown at the 3′ end region of the sense strand in FIG. 44a represents (i) two abasic nucleosides provided as the penultimate and terminal nucleosides at the 3′ end region of the sense strand, (ii) wherein a 3′-3′ reversed linkage is provided between the antepenultimate nucleoside (namely A at position 21 of the sense strand, wherein position 1 is the terminal 5′ nucleoside of the sense strand, namely terminal U at the 5′end region of the sense strand) and the adjacent penultimate abasic residue of the sense strand, and (iii) the linkage between the terminal and penultimate abasic nucleosides is 5′-3′ when reading towards the 3′ end region comprising the terminal and penultimate abasic nucleosides.

For the sense strand of FIG. 44a, when reading from position 1 of the sense strand (which is the terminal 5′ nucleoside of the sense strand, namely terminal U at the 5′end region of the sense strand), then: (i) the nucleosides at positions 1 to 6, 8, and 12 to 21 have sugars that are 2′ O-methyl modified, (ii) the nucleosides at positions 7, and 9 to 11 have sugars that are 2′ F modified, (iii) the abasic nucleosides have sugars that have H at positions 1 and 2.

For the antisense strand of FIG. 44a, when reading from position 1 of the antisense strand (which is the terminal 5′ nucleoside of the antisense strand, namely terminal U at the 5′end region of the antisense strand), then: (i) the nucleosides at positions 1, 3 to 5, 7, 8, 10 to 13, 15, 17 to 23 have sugars that are 2′ O-methyl modified, (ii) the nucleosides at positions 2, 6, 9, 14, 16 have sugars that are 2′ F modified.

FIG. 44b shows the underlying nucleoside sequences for the sense (SS) and antisense (AS) strands of constructs ETX023, ETX024 as described herein. For both ETX023 and ETX024 a galnac linker is attached to the 3′ end region of the sense strand in use (not depicted in FIG. 44b). For ETX023 the galnac linker is attached and as shown in FIG. 30. For ETX024 the galnac linker is attached and as shown in FIG. 32.

iaia as shown at the 5′ end region of the sense strand in FIG. 47b represents (i) two abasic nucleosides provided as the penultimate and terminal nucleosides at the 5′ end region of the sense strand, (ii) wherein a 5′-5′ reversed linkage is provided between the antepenultimate nucleoside (namely U at position 1 of the sense strand, not including the iaia motif at the 5′ end region of the sense strand in the nucleoside position numbering on the sense strand) and the adjacent penultimate abasic residue of the sense strand, and (iii) the linkage between the terminal and penultimate abasic nucleosides is 3′-5′ when reading towards the 5′ end region comprising the terminal and penultimate abasic nucleosides.

For the sense strand of FIG. 44b, when reading from position 1 of the sense strand (which is the terminal 5′ nucleoside of the sense strand, namely terminal U at the 5′end region of the sense strand, not including the iaia motif at the 5′ end region of the sense strand in the nucleoside position numbering on the sense strand), then: (i) the nucleosides at positions 1 to 6, 8, and 12 to 21 have sugars that are 2′ O-methyl modified, (ii) the nucleosides at positions 7, and 9 to 11 have sugars that are 2′ F modified, (iii) the abasic nucleosides have sugars that have H at positions 1 and 2.

For the antisense strand of FIG. 44b, when reading from position 1 of the antisense strand (which is the terminal 5′ nucleoside of the antisense strand, namely terminal U at the 5′end region of the antisense strand), then: (i) the nucleosides at positions 1, 3 to 5, 7, 8, 10 to 13, 15, 17 to 23 have sugars that are 2′ O-methyl modified, (ii) the nucleosides at positions 2, 6, 9, 14, 16 have sugars that are 2′ F modified.

FIG. 45: Results of dose-response experiments for inhibition of HCII or ZPI mRNA expression in human Huh7 cells. Points represent mean relative expression of HCII or ZPI mRNA compared to untreated wells after treatment with siRNA construct at the indicated concentrations on the x-axis. Error bars represent standard deviation of the mean. Dotted curves represent 95% confidence intervals. Dotted lines and shaded areas represent the mean relative expression+/−standard deviation from untreated wells on the same plate.

### SUMMARY OF THE INVENTION

A nucleic acid for inhibiting expression of a target gene, comprising a duplex region that comprises:
a first strand that is at least partially complementary to a portion of RNA transcribed from the target gene, and a second strand that is at least partially complementary to the first strand,
wherein the second strand comprises 2 consecutive abasic nucleosides in the 5′ terminal region of the second strand, wherein one such abasic nucleoside is a terminal nucleoside at the 5′ terminal region of the second strand and the other abasic nucleoside is a penultimate nucleoside at the 5′ terminal region of the second strand, wherein:
(a) said penultimate abasic nucleoside is connected to an adjacent first basic nucleoside in an adjacent 5′ near terminal region through a reversed internucleoside linkage;
(b) the reversed linkage is a 5-5′ reversed linkage; and
(c) the linkage between the terminal and penultimate abasic nucleosides is 3′-5′ when reading towards the terminus comprising the terminal and penultimate abasic nucleosides.

A nucleic acid for inhibiting expression of a target gene, comprising a duplex region that comprises:
a first strand that is at least partially complementary to a portion of RNA transcribed from the target gene, and a second strand that is at least partially complementary to the first strand,
wherein the second strand comprises 2 consecutive abasic nucleosides preferably in an overhang in the 3′ terminal region of the second strand, wherein one such abasic nucleoside is a terminal nucleoside at the 3′ terminal region of the second strand and the other abasic nucleoside is a penultimate nucleoside at the 3′ terminal region of the second strand, wherein:
(a) said penultimate abasic nucleoside is connected to an adjacent first basic nucleoside in an adjacent 3′ near terminal region through a reversed internucleoside linkage;
(b) the reversed linkage is a 3-3′ reversed linkage; and
(c) the linkage between the terminal and penultimate abasic nucleosides is 5′-3′ when reading towards the terminus comprising the terminal and penultimate abasic nucleosides.

A nucleic acid for inhibiting expression of a target gene, comprising a duplex region that comprises:
a first strand that is at least partially complementary to a portion of RNA transcribed from the target gene, and a second strand that is at least partially complementary to the first strand,
wherein:
(i) preferably the first strand and the second strand each has a length of 23 nucleosides (this length for the second strand includes the two abasic nucleosides);
(ii) the second strand comprises 2 consecutive abasic nucleosides in the 5′ terminal region of the second strand, wherein one such abasic nucleoside is a terminal nucleoside at the 5′ terminal region of the second strand and the other abasic nucleoside is a penultimate nucleoside at the 5′ terminal region of the second strand, wherein:
   (a) said penultimate abasic nucleoside is connected to an adjacent first basic nucleoside in an adjacent 5′ near terminal region through a reversed internucleoside linkage; and
   (b) the reversed linkage is a 5-5′ reversed linkage; and
   (c) the linkage between the terminal and penultimate abasic nucleosides is 3-′5′ when reading towards the terminus comprising the terminal and penultimate abasic nucleosides;
(iii) two phosphorothioate internucleoside linkages are respectively present between three consecutive positions in said 5′ near terminal region of the second strand, wherein a first phosphorothioate internucleoside linkage is present between said first basic nucleoside of (a) and an adjacent second basic nucleoside in said 5′ near terminal region of the second strand, and a second phosphorothioate internucleoside linkage is present between said second basic nucleoside and an adjacent third basic nucleoside in said 5′ near terminal region of the second strand;
(iv) two phosphorothioate internucleoside linkages are respectively present between three consecutive positions in both 5′ and 3′ terminal regions of the first strand, whereby a terminal nucleoside respectively at each of the 5′ and 3′ terminal regions of said first strand is each attached to a respective 5′ and 3′ adjacent penultimate nucleoside by a phosphorothioate internucleoside linkage, and each 5′ and 3′ penultimate nucleoside is attached to a respective 5′ and 3′ adjacent antepenultimate nucleoside by a phosphorothioate internucleoside linkage;
and
(v) the second strand of the nucleic acid is conjugated directly or indirectly to the one or more ligand moieties at the 3′ terminal region of the second strand.

A nucleic acid for inhibiting expression of a target gene, comprising a duplex region that comprises:
a first strand that is at least partially complementary to a portion of RNA transcribed from the target gene, and a second strand that is at least partially complementary to the first strand,
wherein:
(i) preferably the first strand and the second strand each has a length of 23 nucleosides (this length for the second strand includes the two abasic nucleosides);
(ii) the second strand comprises 2 consecutive abasic nucleosides in the 3′ terminal region of the second strand, wherein one such abasic nucleoside is a terminal nucleoside at the 3′ terminal region of the second strand and the other abasic nucleoside is a penultimate nucleoside at the 3′ terminal region of the second strand, wherein:
   (a) said penultimate abasic nucleoside is connected to an adjacent first basic nucleoside in an adjacent 3′ near terminal region through a reversed internucleoside linkage; and
   (b) the reversed linkage is a 3-3′ reversed linkage; and
   (c) the linkage between the terminal and penultimate abasic nucleosides is 5-′3′ when reading towards the terminus comprising the terminal and penultimate abasic nucleosides;
(iii) two phosphorothioate internucleoside linkages are respectively present between three consecutive positions in said 3′ near terminal region of the second strand, wherein a first phosphorothioate internucleoside linkage is present between said first basic nucleoside of (a) and an adjacent second basic nucleoside in said 3′ near terminal region of the second strand, and a second phosphorothioate internucleoside linkage is present between said second basic nucleoside and an adjacent third basic nucleoside in said 3′ near terminal region of the second strand;
(iv) two phosphorothioate internucleoside linkages are respectively present between three consecutive positions in both 5′ and 3′ terminal regions of the first strand, whereby a terminal nucleoside respectively at each of the 5′ and 3′ terminal regions of said first strand is each attached to a respective 5′ and 3′ adjacent penultimate nucleoside by a phosphorothioate internucleoside linkage, and each 5′ and 3′ penultimate nucleoside is attached to a respective 5′ and 3′ adjacent antepenultimate nucleoside by a phosphorothioate internucleoside linkage;
and
(v) the second strand of the nucleic acid is conjugated directly or indirectly to the one or more ligand moieties at the 5′ terminal region of the second strand.

A nucleic acid for inhibiting expression of a target gene, comprising a duplex region that comprises:
a first strand that is at least partially complementary to a portion of RNA transcribed from the target gene, and a second strand that is at least partially complementary to the first strand,
wherein the second strand comprises 2 consecutive abasic nucleosides in the 5′ terminal region of the second strand present as the following 5′ terminal motif

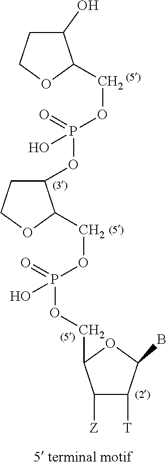

wherein:
B represents a nucleoside base,
T represent H, OH or a 2′ ribose modification,
Z represents the remaining nucleosides of said second strand.

A nucleic acid for inhibiting expression of a target gene, comprising a duplex region that comprises:
a first strand that is at least partially complementary to a portion of RNA transcribed from the target gene, and a second strand that is at least partially complementary to the first strand,
wherein the second strand comprises 2 consecutive abasic nucleosides in the 5′ terminal region of the second strand present as the following 5′ terminal motif

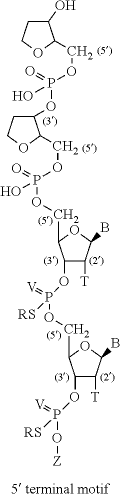

wherein:
B represents a nucleoside base,
T represent H, OH or a 2′ ribose modification,
V represent O or S (preferably O),
R represent H or C1-4 alkyl (preferably H),
Z represents the remaining nucleosides of said second strand,
more preferably the following 5′ terminal motif

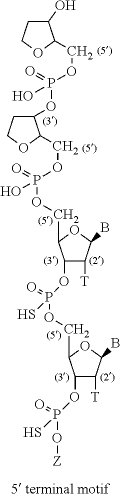

wherein:
B represents a nucleoside base,
T represent H, OH or a 2′ ribose modification,
Z represents the remaining nucleosides of said second strand.

A nucleic acid for inhibiting expression of a target gene, comprising a duplex region that comprises:
a first strand that is at least partially complementary to a portion of RNA transcribed from the target gene, and a second strand that is at least partially complementary to the first strand,
wherein the second strand comprises 2 consecutive abasic nucleosides in the 5′ terminal region of the second strand present as the following 5′ terminal motif

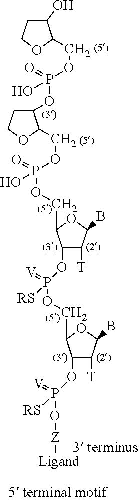

wherein:
B represents a nucleoside base,
T represent H, OH or a 2′ ribose modification,
V represent O or S (preferably O),
R represent H or C₁₋₄ alkyl (preferably H),
Z comprises 11 to 26 contiguous nucleosides, preferably 15 to 21 contiguous nucleosides, and more preferably 19 contiguous nucleosides,
more preferably the following 5′ terminal motif

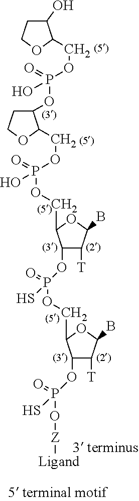

wherein:
B represents a nucleoside base,
T represent H, OH or a 2′ ribose modification,
Z comprises 11 to 26 contiguous nucleosides, preferably 15 to 21 contiguous nucleosides, and more preferably 19 contiguous nucleosides.

A nucleic acid for inhibiting expression of a target gene, comprising a duplex region that comprises:
a first strand that is at least partially complementary to a portion of RNA transcribed from the target gene, and a second strand that is at least partially complementary to the first strand,
wherein the second strand comprises 2 consecutive abasic nucleosides in the 3′ terminal region of the second strand present as the following 3′ terminal motif

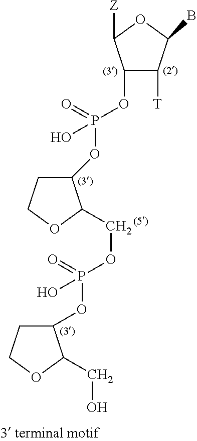

wherein:
B represents a nucleoside base,
T represent H, OH or a 2′ ribose modification,
Z represents the remaining nucleosides of said second strand.

A nucleic acid for inhibiting expression of a target gene, comprising a duplex region that comprises:
a first strand that is at least partially complementary to a portion of RNA transcribed from the target gene, and a second strand that is at least partially complementary to the first strand,
wherein the second strand comprises 2 consecutive abasic nucleosides in the 3′ terminal region of the second strand present as the following 3′ terminal motif

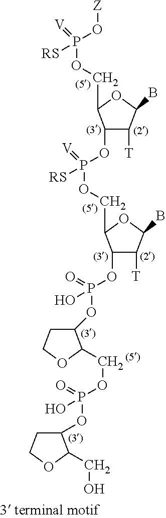

wherein:
B represents a nucleoside base,
T represent H, OH or a 2′ ribose modification,
V represent O or S (preferably O),
R represent H or C₁₋₄ alkyl (preferably H),
Z represents the remaining nucleosides of said second strand,
more preferably the following 3′ terminal motif

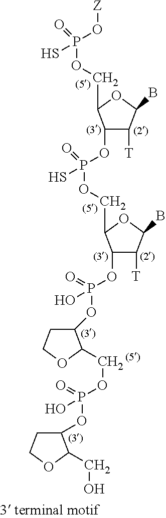

wherein:
B represents a nucleoside base,
T represent H, OH or a 2′ ribose modification,
Z represents the remaining nucleosides of said second strand.

A nucleic acid for inhibiting expression of a target gene, comprising a duplex region that comprises:
a first strand that is at least partially complementary to a portion of RNA transcribed from the target gene, and a second strand that is at least partially complementary to the first strand,
wherein the second strand comprises 2 consecutive abasic nucleosides in the 3′ terminal region of the second strand present as the following 3′ terminal motif

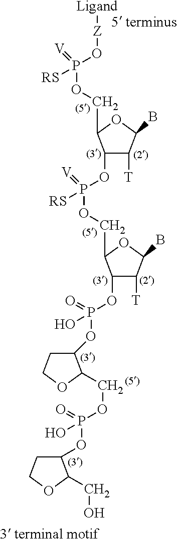

wherein:
B represents a nucleoside base,
T represent H, OH or a 2′ ribose modification,
V represent O or S (preferably O),
R represent H or C₁₋₄ alkyl (preferably H),
Z comprises 11 to 26 contiguous nucleosides, preferably 15 to 21 contiguous nucleosides, and more preferably 19 contiguous nucleosides,
more preferably the following 3′ terminal motif

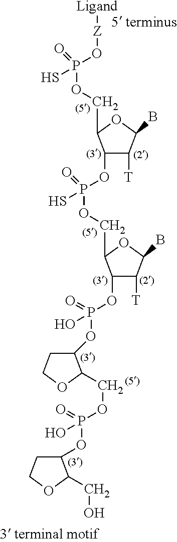

wherein:
B represents a nucleoside base,
T represent H, OH or a 2′ ribose modification,
Z comprises 11 to 26 contiguous nucleosides, preferably 15 to 21 contiguous nucleosides, and more preferably 19 contiguous nucleosides.

### Definitions

The “first strand”, also called the antisense strand or guide strand herein and which can be used interchangeably herein, refers to the nucleic acid strand, e.g. the strand of an iRNA, e.g. a dsRNA, which includes a region that is substantially complementary to a target sequence, e.g. to an mRNA. As used herein, the term “region of complementarity” refers to the region on the antisense strand that is substantially complementary to a sequence, for example a target sequence. Where the region of complementarity is not fully complementary to the target sequence, the mismatches can be in the internal or terminal regions of the molecule. In some embodiments, a double stranded nucleic acid e.g. RNAi agent of the invention includes a nucleoside mismatch in the antisense strand.

The “second strand” (also called the sense strand or passenger strand herein, and which can be used interchangeably herein), refers to the strand of a nucleic acid e.g. iRNA that includes a region that is substantially complementary to a region of the antisense strand as that term is defined herein.

In the context of molecule comprising a nucleic acid provided with a ligand moiety, optionally also with a linker moiety, the nucleic acid of the invention may be referred to as an oligonucleoside moiety.

Oligonucleotides are short nucleic acid polymers. Whilst oligonucleotides contain phosphodiester bonds between the nucleoside thereof (base plus sugar), the present invention is not limited to oligonucleotides always joined by such a phosphodiester bond between adjacent nucleosides, and other oligomers of nucleosides joined by bonds which are bonds other than a phosphodiester bond are contemplated. For example, a bond between nucleosides may be a phosphorothioate bond. Therefore, the term “oligonucleoside” as used herein covers both oligonucleotides and other oligomers of nucleosides. An oligonucleoside which is a nucleic acid having at least a portion which is an oligonucleotide is preferred according to the present invention. An oligonucleoside having one or more, or a majority of, phosphodiester backbone bonds between nucleosides is also preferred according to the present invention. An oligonucleoside having one or more, or a majority of, phosphodiester backbone bonds between nucleosides, and also having one or more phosphorothioate backbone bonds between nucleosides (typically in a terminal region of the first and/or second strands) is also preferred according to the present invention.

In some embodiments, a double stranded nucleic acid e.g. RNAi agent of the invention includes a nucleoside mismatch in the sense strand. In some embodiments, the nucleoside mismatch is, for example, within 5, 4, 3, 2, or 1 nucleosides from the 3′-end of the nucleic acid e.g. iRNA.

In another embodiment, the nucleoside mismatch is, for example, in the 3′-terminal nucleoside of the nucleic acid e.g. iRNA.

A “target sequence” (which may also be called a target RNA or a target mRNA) refers to a contiguous portion of the nucleoside sequence of an mRNA molecule formed during the transcription of a gene, including mRNA that is a product of RNA processing of a primary transcription product.

The target sequence may be from about 10-35 nucleosides in length, e.g., about 15-30 nucleosides in length. For example, the target sequence can be from about 15-30 nucleosides, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-component 22 nucleosides in length. Ranges and lengths intermediate to the above recited ranges and lengths are also contemplated to be part of the invention.

The term “ribonucleoside” or “nucleoside” can also refer to a modified nucleoside, as further detailed below.

A nucleic acid can be a DNA or an RNA, and can comprise modified nucleosides. RNA is a preferred nucleic acid.

The terms “iRNA”, “RNAi agent,” and “iRNA agent,” “RNA interference agent” as used interchangeably herein, refer to an agent that contains RNA, and which mediates the targeted cleavage of an RNA transcript via an RNA-induced silencing complex (RISC) pathway. iRNA directs the sequence-specific degradation of mRNA through RNA interference (RNAi).

A double stranded RNA is referred to herein as a “double stranded RNAi agent,” “double stranded RNA (dsRNA) molecule,” “dsRNA agent,” or “dsRNA”, which refers to a complex of ribonucleic acid molecules, having a duplex structure comprising two anti-parallel and substantially complementary nucleic acid strands, referred to as having “sense” and “antisense” orientations with respect to a target RNA.

The majority of nucleosides of each strand of the nucleic acid, e.g. a dsRNA molecule, are preferably ribonucleosides, but in that case each or both strands can also include one or more non-ribonucleosides, e.g., a deoxyribonucleoside or a modified nucleoside. In addition, as used in this specification, an “iRNA” may include ribonucleosides with chemical modifications.

The term “modified nucleoside” refers to a nucleoside having, independently, a modified sugar moiety, a modified internucleoside linkage, or modified nucleobase, or any combination thereof. Thus, the term modified nucleoside encompasses substitutions, additions or removal of, e.g., a functional group or atom, to internucleoside linkages, sugar moieties, or nucleobases. Any such modifications, as used in a siRNA type molecule, are encompassed by “iRNA” or “RNAi agent” for the purposes of this specification and claims.

The duplex region of a nucleic acid of the invention e.g. a dsRNA may range from about 9 to 40 base pairs in length such as 9 to 36 base pairs in length, e.g., about 15-30 base pairs in length, for example, about 9, 10, 11, 12, 13, 14, 15, 16, 17, 18, 19, 20, 21, 22, 23, 24, 25, 26, 27, 28, 29, 30, 31, 32, 33, 34, 35, or 36 base pairs in length, such as about 15-30, 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24, 20-23, 20-22, 20-21, 21-30, 21-29, 21-28, 21-27, 21-26, 21-25, 21-24, 21-23, or 21-22 base pairs in length.

The two strands forming the duplex structure may be different portions of one larger molecule, or they may be separate molecules e.g. RNA molecules.

The term “nucleoside overhang” refers to at least one unpaired nucleoside that extends from the duplex structure of a double stranded nucleic acid. A ds nucleic acid can comprise an overhang of at least one nucleoside; alternatively the overhang can comprise at least two nucleosides, at least three nucleosides, at least four nucleosides, at least five nucleosides or more. A nucleoside overhang can comprise or consist of a nucleoside/nucleoside analog, including a deoxynucleoside. The overhang(s) can be on the sense strand, the antisense strand, or any combination thereof. Furthermore, the nucleoside(s) of an overhang can be present on the 5′-end, 3-end, or both ends of either an antisense or sense strand.

In certain embodiments, the antisense strand has a 1-10 nucleoside, e.g., 0-3, 1-3, 2-4, 2-5, 4-10, 5-10, 1, 2, 3, 4, 5, 6, 7, 8, 9, or 10 nucleoside, overhang at the 3′-end or the 5′-end.

“Blunt” or “blunt end” means that there are no unpaired nucleosides at that end of the double stranded nucleic acid, i.e., no nucleoside overhang. The nucleic acids of the invention include those with no nucleoside overhang at one end or with no nucleoside overhangs at either end.

Unless otherwise indicated, the term “complementary,” when used to describe a first nucleoside sequence in relation to a second nucleoside sequence, refers to the ability of an oligonucleoside or polynucleoside comprising the first nucleoside sequence to hybridize and form a duplex structure under certain conditions with an oligonucleoside or polynucleoside comprising the second nucleoside sequence, as will be understood by the skilled person. Such conditions can, for example, be stringent conditions, where stringent conditions can include: 400 mM NaCl, 40 mM PIPES pH 6.4, 1 mM EDTA, 50° C. or 70° C. for 12-16 hours followed by washing (see, e.g., “Molecular Cloning: A Laboratory Manual, Sambrook, et al. (1989) Cold Spring Harbor Laboratory Press).

Complementary sequences within nucleic acid e.g. a dsRNA, as described herein, include base-pairing of the oligonucleoside or polynucleoside comprising a first nucleoside sequence to an oligonucleoside or polynucleoside comprising a second nucleoside sequence over the entire length of one or both nucleoside sequences. Such sequences can be referred to as “fully complementary” with respect to each other herein. However, where a first sequence is referred to as “substantially complementary” with respect to a second sequence herein, the two sequences can be fully complementary, or they can form one or more mismatched base pairs, such as 2, 4, or 5 mismatched base pairs, but preferably not more than 5, while retaining the ability to hybridize under the conditions most relevant to their ultimate application, e.g., inhibition of gene expression via a RISC pathway. Overhangs shall not be regarded as mismatches with regard to the determination of complementarity. For example, a nucleic acid e.g. dsRNA comprising one oligonucleoside 17 nucleosides in length and another oligonucleoside 19 nucleosides in length, wherein the longer oligonucleoside comprises a sequence of 17 nucleosides that is fully complementary to the shorter oligonucleoside, can yet be referred to as “fully complementary”.

“Complementary” sequences, as used herein, can also include, or be formed entirely from, non-Watson-Crick base pairs or base pairs formed from non-natural and modified nucleosides, in so far as the above requirements with respect to their ability to hybridize are fulfilled. Such non-Watson-Crick base pairs include, but are not limited to, G:U Wobble or Hoogstein base pairing.

The terms “complementary,” “fully complementary” and “substantially complementary” herein can be used with respect to the base matching between the sense strand and the antisense strand of a nucleic acid eg dsRNA, or between the antisense strand of a double stranded nucleic acid e.g. RNAi agent and a target sequence.

As used herein, a nucleic acid or polynucleoside that is “substantially complementary” to at least part of a messenger RNA (mRNA) refers to a polynucleoside that is substantially complementary to a contiguous portion of the mRNA of interest (e.g., an mRNA encoding a gene). For example, a polynucleoside is complementary to at least a part of an mRNA of a gene of interest if the sequence is substantially complementary to a non-interrupted portion of an mRNA encoding that gene.

Accordingly, in some preferred embodiments, the sense strand polynucleosides and the antisense polynucleosides disclosed herein are fully complementary to the target gene sequence.

In other embodiments, the antisense polynucleosides disclosed herein are substantially complementary to a target RNA sequence and comprise a contiguous nucleoside sequence which is at least about 80% complementary over its entire length to the equivalent region of the target RNA sequence, such as at least about 85%, 86%, 87%, 88%, 89%, about 90%, 91%, 92%, 93%, 94%, 95%, 96%, 97%, 98%, or 99% complementary or 100% complementary.

In some embodiments, a nucleic acid e.g. an iRNA of the invention includes a sense strand that is substantially complementary to an antisense polynucleoside which, in turn, is complementary to a target gene sequence and comprises a contiguous nucleoside sequence which is at least about 80% complementary over its entire length to the equivalent region of the nucleoside sequence of the antisense strand, such as about 85%, 86%, 87%, 88%, 89%, 90%, 91%, 92%, 93%, 94%, 95%, 96%, 97%, 98%, or 99% complementary, or 100% complementary.

In some embodiments, a nucleic acid e.g. an iRNA of the invention includes an antisense strand that is substantially complementary to the target sequence and comprises a contiguous nucleoside sequence which is at least 80% complementary over its entire length to the target sequence such as about 85%, 86%, 87%, 88%, 89%, 90%, 91%, 92%, 93%, 94%, 95%, 96%, 97%, 98%, or 99% complementary, or 100% complementary.

As used herein, a “subject” is an animal, such as a mammal, including a primate (such as a human, a non-human primate, e.g., a monkey, and a chimpanzee), or a non-primate or a bird that expresses the target gene, either endogenously or heterologously, when the target gene sequence has sufficient complementarity to the nucleic acid e.g. iRNA agent to promote target knockdown. In certain preferred embodiments, the subject is a human.

The terms “treating” or “treatment” refer to a beneficial or desired result including, but not limited to, alleviation or amelioration of one or more symptoms associated with gene expression. “Treatment” can also mean prolonging survival as compared to expected survival in the absence of treatment. Treatment can include prevention of development of co-morbidities, e.g., reduced liver damage in a subject with a hepatic infection.

“Therapeutically effective amount,” as used herein, is intended to include the amount of a nucleic acid e.g. an iRNA that, when administered to a patient for treating a subject having disease, is sufficient to effect treatment of the disease (e.g., by diminishing, ameliorating or maintaining the existing disease or one or more symptoms of disease or its related comorbidities).

The phrase “pharmaceutically acceptable” is employed herein to refer to compounds, materials, compositions, or dosage forms which are suitable for use in contact with the tissues of human subjects and animal subjects without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition, or vehicle, such as a liquid or solid filler, diluent, excipient, manufacturing aid or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject being treated.

Where a value or range of values of a parameter are recited, it is intended that values and ranges intermediate to the recited values are also intended to be part of this invention.

The articles “a” and “an” are used herein to refer to one or to more than one (i.e. to at least one) of the grammatical object of the article.

The term “including” is used herein to mean, and is used interchangeably with, the phrase “including but not limited to”.

The term “or” is used herein to mean, and is used interchangeably with, the term “and/or,” unless context clearly indicates otherwise. For example, “sense strand or antisense strand” is understood as “sense strand or antisense strand or sense strand and antisense strand.”

The term “about” is used herein to mean within the typical ranges of tolerances in the art. For example, “about” can be understood as about 2 standard deviations from the mean. In certain embodiments, about means+10%. In certain embodiments, about means+5%. When about is present before a series of numbers or a range, it is understood that “about” can modify each of the numbers in the series or range.

The term “at least” prior to a number or series of numbers is understood to include the number adjacent to the term “at least”, and all subsequent numbers or integers that could logically be included, as clear from context. For example, the number of nucleosides in a nucleic acid molecule must be an integer. For example, “at least 18 nucleosides of a 21 nucleoside nucleic acid molecule” means that 18, 19, 20, or 21 nucleosides have the indicated property. When at least is present before a series of numbers or a range, it is understood that “at least” can modify each of the numbers in the series or range.

As used herein, “no more than” or “less than” is understood as the value adjacent to the phrase and logical lower values or integers, as logical from context, to zero. For example, a duplex with an overhang of “no more than 2 nucleosides” has a 2, 1, or 0 nucleoside overhang. When “no more than” is present before a series of numbers or a range, it is understood that “no more than” can modify each of the numbers in the series or range.

The terminal region of a strand is the last 5 nucleosides from the 5′ or the 3′ end.

Various embodiments of the invention can be combined as determined appropriate by one of skill in the art.

### Abasic Nucleosides

There are 1, e.g. 2, e.g. 3, e.g. 4 or more abasic nucleosides present in the nucleic acid. Abasic nucleosides are modified nucleosides because they lack the base normally seen at position 1 of the sugar moiety. Typically, there will be a hydrogen at position 1 of the sugar moiety of the abasic nucleosides present in a nucleic acid according to the present invention.

The abasic nucleosides are in the terminal region of the second strand, preferably located within the terminal 5 nucleosides of the end of the strand. The terminal region may be the terminal 5 nucleosides, which includes abasic nucleosides.

The second strand may comprise, as preferred features (which are all specifically contemplated in combination unless mutually exclusive):
2, or more than 2, abasic nucleosides in a terminal region of the second strand; and/or
2, or more than 2, abasic nucleosides in either the 5′ or 3′ terminal region of the second strand; and/or
2, or more than 2, abasic nucleosides in either the 5′ or 3′ terminal region of the second strand, wherein the abasic nucleosides are present in an overhang as herein described; and/or
2, or more than 2, consecutive abasic nucleosides in a terminal region of the second strand, wherein preferably one such abasic nucleoside is a terminal nucleoside; and/or
2, or more than 2, consecutive abasic nucleosides in either the 5′ or 3′ terminal region of the second strand, wherein preferably one such abasic nucleoside is a terminal nucleoside in either the 5′ or 3′ terminal region of the second strand; and/or
a reversed internucleoside linkage connects at least one abasic nucleoside to an adjacent basic nucleoside in a terminal region of the second strand; and/or
a reversed internucleoside linkage connects at least one abasic nucleoside to an adjacent basic nucleoside in either the 5′ or 3′ terminal region of the second strand; and/or
an abasic nucleoside as the penultimate nucleoside which is connected via the reversed linkage to the nucleoside which is not the terminal nucleoside (called the antepenultimate nucleoside herein); and/or
abasic nucleosides as the 2 terminal nucleosides connected via a 5′-3′ linkage when reading the strand in the direction towards the terminus comprising the terminal nucleosides;
abasic nucleosides as the 2 terminal nucleosides connected via a 3′-5′ linkage when reading the strand in the direction towards the terminus comprising the terminal nucleosides;
abasic nucleosides as the terminal 2 positions, wherein the penultimate nucleoside is connected via the reversed linkage to the antepenultimate nucleoside, and wherein the reversed linkage is a 5-5′ reversed linkage or a 3′-3′ reversed linkage;
abasic nucleosides as the terminal 2 positions, wherein the penultimate nucleoside is connected via the reversed linkage to the antepenultimate nucleoside, and wherein either
(1) the reversed linkage is a 5-5′ reversed linkage and the linkage between the terminal and penultimate abasic nucleosides is 3′5′ when reading towards the terminus comprising the terminal and penultimate abasic nucleosides; or
(2) the reversed linkage is a 3-3′ reversed linkage and the linkage between the terminal and penultimate abasic nucleosides is 5′3′ when reading towards the terminus comprising the terminal and penultimate abasic nucleosides.

Preferably there is an abasic nucleoside at the terminus of the second strand.

Preferably there are 2 or at least 2 abasic nucleosides in the terminal region of the second strand, preferably at the terminal and penultimate positions.

Preferably 2 or more abasic nucleosides are consecutive, for example all abasic nucleosides may be consecutive. For example, the terminal 1 or terminal 2 or terminal 3 or terminal 4 nucleosides may be abasic nucleosides.

An abasic nucleoside may also be linked to an adjacent nucleoside through a 5′-3′ phosphodiester linkage or reversed linkage unless there is only 1 abasic nucleoside at the terminus, in which case it will have a reversed linkage to the adjacent nucleoside.

A reversed linkage (which may also be referred to as an inverted linkage, which is also seen in the art), comprises either a 5′-5′, a 3′3′, a 3′-2′ or a 2′-3′ phosphodiester linkage between the adjacent sugar moieties of the nucleosides.

Abasic nucleosides which are not terminal will have 2 phosphodiester bonds, one with each adjacent nucleoside, and these may be a reversed linkage or may be a 5′-3 phosphodiester bond or may be one of each.

A preferred embodiment comprises 2 abasic nucleosides at the terminal and penultimate positions of the second strand, and wherein the reversed internucleoside linkage is located between the penultimate (abasic) nucleoside and the antepenultimate nucleoside.

Preferably there are 2 abasic nucleosides at the terminal and penultimate positions of the second strand and the penultimate nucleoside is linked to the antepenultimate nucleoside through a reversed internucleoside linkage and is linked to the terminal nucleoside through a 5′-3′ or 3′-5′ phosphodiester linkage (reading in the direction of the terminus of the molecule).

Different preferred features are as follows:

The reversed internucleoside linkage is a 3′3 reversed linkage. The reversed internucleoside linkage is at a terminal region which is distal to the 5′ terminal phosphate of the second strand.

The reversed internucleoside linkage is a 5′5 reversed linkage. The reversed internucleoside linkage is at a terminal region which is distal to the 3′ terminal hydroxide of the second strand.

Examples of the structures are as follows (where the specific RNA nucleosides shown are not limiting and could be any RNA nucleoside):
A A 3′-3′ reversed bond (and also showing the 5′-3 direction of the last phosphodiester bond between the two abasic molecules reading towards the terminus of the molecule)

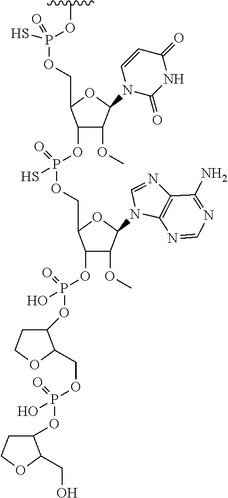 [SMILES: *OP(=O)(S)OCC1O[C@@H](n2ccc(=O)[nH]c2=O)C(OC)C1OP(=O)(S)OCC1O[C@@H](n2cnc3c(N)ncnc32)C(OC)C1OP(=O)(O)OC1CCOC1COP(=O)(O)OC1CCOC1CO]

B Illustrating a 5′-5′ reversed bond (and also showing the 3′-5′ direction of the last phosphodiester bond between the two abasic molecules reading towards the terminus of the molecule)

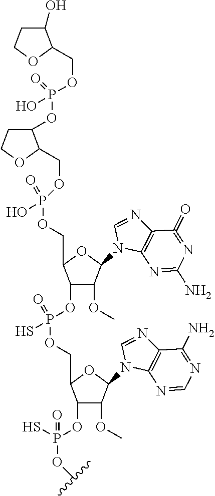

The abasic nucleoside or abasic nucleosides present in the nucleic acid are provided in the presence of a reversed internucleoside linkage or linkages, namely a 5′-5′ or a 3′-3′ reversed internucleoside linkage. A reversed linkage occurs as a result of a change of orientation of an adjacent nucleoside sugar, such that the sugar will have a 3′ 5′ orientation as opposed to the conventional 5′ 3′ orientation (with reference to the numbering of ring atoms on the nucleoside sugars). The abasic nucleoside or nucleosides as present in the nucleic acids of the invention preferably include such inverted nucleoside sugars.

In the case of a terminal nucleoside having an inverted orientation, then this will result in an “inverted” end configuration for the overall nucleic acid. Whilst certain structures drawn and referenced herein are represented using conventional 5′-3′ direction (with reference to the numbering of ring atoms on the nucleoside sugars), it will be appreciated that the presence of a terminal nucleoside having a change of orientation and a proximal 3′-3′ reversed linkage, will result in a nucleic acid having an overall 5′-5′ end structure (i.e. the conventional 3′ end nucleoside becomes a 5′ end nucleoside). Alternatively, it will be appreciated that the presence of a terminal nucleoside having a change of orientation and a proximal 5′-5′ reversed linkage will result in a nucleic acid with an overall 3′-3′ end structure.

The proximal 3′-3′ or 5′-5′ reversed linkage as herein described, may comprise the reversed linkage being directly adjacent/attached to a terminal nucleoside having an inverted orientation, such as a single terminal nucleoside having an inverted orientation. Alternatively, the proximal 3′-3′ or 5′-5′ reversed linkage as herein described, may comprise the reversed linkage being adjacent 2, or more than 2, nucleosides having an inverted orientation, such as 2, or more than 2, terminal region nucleosides having an inverted orientation, such as the terminal and penultimate nucleosides. In this way, the reversed linkage may be attached to a penultimate nucleoside having an inverted orientation. While a skilled addressee will appreciate that inverted orientations as described above can result in nucleic acid molecules having overall 3′-3′ or 5′-5′ end structures as described herein, it will also be appreciated that with the presence of one or more additional reversed linkages and/or nucleosides having an inverted orientation, then the overall nucleic acid may have 3′-5′ end structures corresponding to the conventionally positioned 5′/3′ ends.

In one aspect the nucleic acid may have a 3′-3′ reversed linkage, and the terminal sugar moiety may comprise a 5′ OH rather than a 5′ phosphate group at the 5′ position of that terminal sugar.

A skilled person would therefore clearly understand that 5′-5′, 3′-3′ and 3′-5′ (reading in the direction of that terminus) end variants of the more conventional 5′-3′ structures (with reference to the numbering of ring atoms on the end nucleoside sugars) drawn herein are included in the scope of the disclosure, where a reversed linkage or linkages is/are present.

In the situation of eg a reversed internucleoside linkage and/or one or more nucleosides having an inverted orientation creating an inverted end, and where the relative position of a linkage (eg to a linker) or the location of an internal feature (such as a modified nucleoside) is defined relative to the 5′ or 3′ end of the nucleic acid, then the 5′ or 3′ end is the conventional 5′ or 3′ end which would have existed had a reversed linkage not been in place, and wherein the conventional 5′ or 3′ end is determined by consideration of the directionality of the majority of the internal nucleoside linkages and/or nucleoside orientation within the nucleic acid. It is possible to tell from these internal bonds and/or nucleoside orientation which ends of the nucleic acid would constitute the conventional 5′ and 3′ ends (with reference to the numbering of ring atoms on the end nucleoside sugars) of the molecule absent the reversed linkage.

For example, in the structure shown below there are abasic residues in the first 2 positions located at the “5′” end. Where the terminal nucleoside has an inverted orientation then the “5′” end indicated in the diagram below, which is the conventional 5′ end, can in fact comprise a 3′ OH in view of the inverted nucleoside at the terminal position. Nevertheless the majority of the molecule will comprise conventional internucleoside linkages that run from the 3′ OH of the sugar to the 5′ phosphate of the next sugar, when reading in the standard 5′ [P04] to 3′ [OH] direction of a nucleic acid molecule (with reference to the numbering of ring atoms on the nucleoside sugars), which can be used to determine the conventional 5′ and 3′ ends that would be found absent the inverted end configuration.
A 5′ A-A-Me-Me-Me-Me-Me-Me-F-Me-F—F—F-Me-Me-Me-Me-Me-Me-Me-Me-Me-Me 3′

The reversed bond is preferably located at the end of the nucleic acid eg RNA which is distal to a ligand moiety, such as a GalNAc containing portion, of the molecule.

GalNAc-siRNA constructs with a 5′-GalNAc on the sense strand can have a reversed linkage on the opposite end of the sense strand.

GalNAc-siRNA constructs with a 3′-GalNAc on the sense strand can have a reversed linkage on the opposite end of the sense strand.

### Nucleic Acid Lengths

In one aspect the i) the first strand of the nucleic acid has a length in the range of 15 to 30 nucleosides, preferably 19 to 25 nucleosides, more preferably 23 or 25; and/or
ii) the second strand of the nucleic acid has a length in the range of 15 to 30 nucleosides, preferably 19 to 25 nucleosides, more preferably 23.

Generally, the duplex structure of the nucleic acid e.g. an iRNA is about 15 to 30 base pairs in length, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 base pairs in length. Ranges and lengths intermediate to the above recited ranges and lengths are also contemplated to be part of the invention.

Similarly, the region of complementarity of an antisense sequence to a target sequence and/or the region of complementarity of an antisense sequence to a sense sequence is about 15 to 30 nucleosides in length, e.g., 15-29, 15-28, 15-27, 15-26, 15-25, 15-24, 15-23, 15-22, 15-21, 15-20, 15-19, 15-18, 15-17, 18-30, 18-29, 18-28, 18-27, 18-26, 18-25, 18-24, 18-23, 18-22, 18-21, 18-20, 19-30, 19-29, 19-28, 19-27, 19-26, 19-25, 19-24, 19-23, 19-22, 19-21, 19-20, 20-30, 20-29, 20-28, 20-27, 20-26, 20-25, 20-24,20-23, 20-22, 20-21, 21-30, 21-29, 21-28, 21-27, 21-26, 21-25, 21-24, 21-23, or 21-22 nucleosides in length. Ranges and lengths intermediate to the above recited ranges and lengths are also contemplated to be part of the invention.

In certain preferred embodiments, the region of complementarity of an antisense sequence to a target sequence and/or the region of complementarity of an antisense sequence to a sense sequence is at least 17 nucleosides in length. For example, the region of complementarity between the antisense strand and the target is 19 to 21 nucleosides in length, for example, the region of complementarity is 21 nucleosides in length.

In preferred embodiments, each strand is no more than 30 nucleosides in length.

A nucleic acid e.g. a dsRNA as described herein can further include one or more single-stranded nucleoside overhangs e.g., 1-4, 2-4, 1-3, 2-3, 1, 2, 3, or 4 nucleosides. A nucleoside overhang can comprise or consist of a nucleoside/nucleoside analog, including a deoxynucleoside/nucleoside. The overhang(s) can be on the sense strand, the antisense strand, or any combination thereof. Furthermore, the nucleoside(s) of an overhang can be present on the 5′-end, 3′-end, or both ends of an antisense or sense strand of a nucleic acid e.g. a dsRNA.

In certain preferred embodiments, at least one strand comprises a 3′ overhang of at least 1 nucleoside, e.g., at least one strand comprises a 3′ overhang of at least 2 nucleosides. The overhang is suitably on the antisense/guide strand and/or the sense/passenger strand.

### Nucleic Acid Modifications

In certain embodiments, the nucleic acid e.g. an RNA of the invention e.g., a dsRNA, does not comprise further modifications (beyond the required abasic modifications), e.g., chemical modifications or conjugations known in the art and described herein.

In other preferred embodiments, the nucleic acid e.g. RNA of the invention, e.g., a dsRNA, is further chemically modified (beyond the abasic modifications) to enhance stability or other beneficial characteristics.

In certain embodiments of the invention, substantially all of the nucleosides are modified.

The nucleic acids featured in the invention can be synthesized or modified by methods well established in the art, such as those described in “Current protocols in nucleic acid chemistry,” Beaucage, S. L. et al. (Edrs.), John Wiley & Sons, Inc., New York, NY, USA, which is hereby incorporated herein by reference.

Modifications include, for example, end modifications, e.g., 5′-end modifications (phosphorylation, conjugation, inverted linkages) or 3′-end modifications (conjugation, DNA nucleosides within an RNA, or RNA nucleosides within a DNA, inverted linkages, etc.); base modifications, e.g., replacement with stabilizing bases, destabilizing bases, or bases that base pair with an expanded repertoire of partners, conjugated bases; sugar modifications (e.g., at the 2-position or 4′-position) or replacement of the sugar; or backbone modifications, including modification or replacement of the phosphodiester linkages.

Specific examples of nucleic acids such as iRNA compounds useful in the embodiments described herein include, but are not limited to RNAs containing modified backbones or no natural internucleoside linkages. Nucleic acids such as RNAs having modified backbones include, among others, those that do not have a phosphorus atom in the backbone. For the purposes of this specification, and as sometimes referenced in the art, modified nucleic acids e.g. RNAs that do not have a phosphorus atom in their internucleoside backbone can also be considered to be oligonucleosides. In some embodiments, a modified nucleic acid e.g. an iRNA will have a phosphorus atom in its internucleoside backbone.

Modified nucleic acid e.g. RNA backbones include, for example, phosphorothioates, chiral phosphorothioates, phosphorodithioates, phosphotriesters, aminoalkylphosphotriesters, methyl and other alkyl phosphonates including 3′-alkylene phosphonates and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates having normal 3′-5′ linkages, 2′-5′-linked analogs of these, and those having inverted polarity wherein the adjacent pairs of nucleoside units are linked 5′-3′ or 5′-2′. Various salts, mixed salts and free acid forms are also included.

Modified nucleic acids e.g. RNAs can also contain one or more substituted sugar moieties. The nucleic acids e.g. iRNAs, e.g., dsRNAs, featured herein can include one of the following at the 2′-position: OH; F; 0-, S—, or N-alkyl; 0-, S—, or N-alkenyl; 0-, S- or N-alkynyl; or O-alkyl-O-alkyl, wherein the alkyl, alkenyl and alkynyl can be substituted or unsubstituted. 2′ O-methyl and 2′-F are preferred modifications.

In certain preferred embodiments, the nucleic acid comprises at least one modified nucleoside.

The nucleic acid of the invention may comprise one or more modified nucleosides on the first strand and/or the second strand.

In some embodiments, substantially all of the nucleosides of the sense strand and all of the nucleosides of the antisense strand comprise a modification.

In some embodiments, all of the nucleosides of the sense strand and substantially all of the nucleosides of the antisense strand comprise a modification.

In some embodiments, all of the nucleosides of the sense strand and all of the nucleosides of the antisense strand comprise a modification.

In one embodiment, at least one of the modified nucleosides is selected from the group consisting of a deoxy-nucleoside, a 3′-terminal deoxy-thymine (dT) nucleoside, a 2′-0-methyl modified nucleoside (also called herein 2′-Me, where Me is a methoxy), a 2′-fluoro modified nucleoside, a 2′-deoxy-modified nucleoside, a locked nucleoside, an unlocked nucleoside, a conformationally restricted nucleoside, a constrained ethyl nucleoside, an abasic nucleoside, a 2′-amino-modified nucleoside, a 2′-O-allyl-modified nucleoside, 2′-C-alkyl-modified nucleoside, 2′-hydroxyl-modified nucleoside, a 2′-methoxyethyl modified nucleoside, a 2-0-alkyl-modified nucleoside, a morpholino nucleoside, a phosphoramidate, a non-natural base comprising nucleoside, a tetrahydropyran modified nucleoside, a 1,5-anhydrohexitol modified nucleoside, a cyclohexenyl modified nucleoside, a nucleoside comprising a phosphorothioate group, a nucleoside comprising a methylphosphonate group, a nucleoside comprising a 5′-phosphate, and a nucleoside comprising a 5′-phosphate mimic. In another embodiment, the modified nucleosides comprise a short sequence of 3′-terminal deoxy-thymine nucleosides (dT).

Modifications on the nucleosides may preferably be selected from the group including, but not limited to, LNA, HNA, CeNA, 2-methoxyethyl, 2′-0-alkyl, 2-0-allyl, 2′-C-allyl, 2′-fluoro, 2′-deoxy, 2′-hydroxyl, and combinations thereof. In another embodiment, the modifications on the nucleosides are 2-0-methyl (“2-Me”) or 2′-fluoro modifications.

One preferred modification is a modification at the 2′-OH group of the ribose sugar, optionally selected from 2′-Me or 2′-F modifications.

Preferred nucleic acid comprise one or more nucleosides on the first strand and/or the second strand which are modified, to form modified nucleosides, as follows:

A nucleic acid wherein the modification is a modification at the 2′-OH group of the ribose sugar, optionally selected from 2′-Me or 2′-F modifications.

A nucleic acid wherein the first strand comprises a 2′-F at any of position 14, position 2, position 6, or any combination thereof, counting from position 1 of said first strand.

A nucleic acid wherein the second strand comprises a 2′-F modification at position 7 and/or 9, and/or 11, and/or 13, counting from position 1 of said second strand.

A nucleic acid wherein the second strand comprises a 2′-F modification at position 7 and 9 and 11 counting from position 1 of said second strand.

A nucleic acid wherein the first and second strand each comprise 2′-Me and 2′-F modifications.

A nucleic acid wherein the nucleic acid comprises at least one thermally destabilizing modification, suitably at one or more of positions 1 to 9 of the first strand, and/or at one or more of positions on the second strand aligned with positions 1 to 9 of the first strand, wherein the destabilizing modification is selected from a modified unlocked nucleic acid (IMUNA) and a glycol nucleic acid (GNA), preferably a glycol nucleic acid. The nucleic acid may be a double stranded molecule, preferably double stranded RNA, which has a melting temperature in the range of about 40 to 80° C. The nucleic acid may comprise at least one thermally destabilizing modification at position 7 of the first strand.

A nucleic acid wherein the nucleic acid comprises 3 or more 2′-F modifications at positions 7 to 13 of the second strand, such as 4, 5, 6 or 7 2′-F modifications at positions 7 to 13 of the second strand, counting from position 1 of said second strand.

A nucleic acid wherein said second strand comprises at least 3, such as 4, 5 or 6, 2′-Me modifications at positions 1 to 6 of the second strand, counting from position 1 of said second strand.

A nucleic acid wherein said first strand comprises at least 5 2′-Me consecutive modifications at the 3′ terminal region, preferably including the terminal nucleoside at the 3′ terminal region, or at least within 1 or 2 nucleosides from the terminal nucleoside at the 3′ terminal region.

A nucleic acid wherein said first strand comprises 7 2′-Me consecutive modifications at the 3′ terminal region, preferably including the terminal nucleoside at the 3′ terminal region.

Preferred modification patterns include:
A nucleic acid wherein the second strand includes the following modification pattern:

N^{A}—(N)₃₋₅—N^{B}
wherein N represents a nucleoside with a first modification;
N^{A} represents a nucleoside with a second modification different to the first modification of N;
N^{B} represents a nucleoside with a third modification different to the first modification of N, but either the same or different to the second modification of N^{A}; and
wherein said pattern has a 5′ to 3′ directionality along the second strand.

A nucleic acid wherein the second strand includes the following modification pattern:

N^{A}—(N)₃—N^{B}.

A nucleic acid wherein the second strand includes the following modification pattern:

N^{A}—(N)₅—N^{B}.

A nucleic acid wherein the second strand includes the following modification pattern:

Me-(F)₃-Me.

A nucleic acid wherein the second strand includes the following modification pattern:

Me-(F)₅-Me.

A nucleic acid wherein the second strand includes the following modification pattern:

N^{C}—N^{A}—(N)₃₋₅—N^{B}—N^{D}

wherein N_{C} and N^{D}, which may be the same or different, respectively denote a plurality of 5′ and 3′ terminal region chemically modified nucleosides, wherein at least N^{C} comprises at least two differently modified nucleosides.

A nucleic acid wherein N^{D} comprises at least two differently modified nucleosides, or a plurality of nucleosides each having the same modification, preferably 2′-Me consecutive modifications.

A nucleic acid wherein the second strand includes the following modification pattern:
5′ A-A-Me-Me-Me-Me-Me-Me-F-Me-F—F—F-Me-Me-Me-Me-Me-Me-Me-Me-Me-Me 3′
   where A represents an abasic modification.

A nucleic acid wherein the second strand includes the following modification pattern:
5′ A-A-Me-Me-F-Me-F-Me-F-Me-F—F—F-Me-F-Me-Me-F-Me-F-Me-Me-Me 3′
   where A represents an abasic modification.

A nucleic acid wherein the second strand includes the following modification pattern:
5′ Me-Me-Me-Me-Me-Me-F-Me-F—F—F-Me-Me-Me-Me-Me-Me-Me-Me-Me-Me-A-A 3′
   where A represents an abasic modification.

A nucleic acid wherein the second strand includes the following modification pattern:
5′ Me-Me-F-Me-F-Me-F-Me-F—F—F-Me-F-Me-Me-F-Me-F-Me-Me-Me-A-A 3′
   where A represents an abasic modification.

A nucleic acid wherein the first strand includes the following modification pattern:

M^{A}-(M)₃₋₅-M^{B}
wherein M represents a nucleoside with a first modification and wherein typically (M)₃₋₅ are substantially aligned with (N)₃₋₅ in said second strand;
M^{A} represents a nucleoside with a second modification different to the first modification of M;
M^{B} represents a nucleoside with a third modification different to the first modification of M, but either the same or different to the second modification of M^{A}.

A nucleic acid, wherein the first strand includes the following modification pattern:

M^{A}-(M)₃-M^{B}.

A nucleic acid, wherein the first strand includes the following modification pattern:

M^{A}-(M)₄-M^{B}.

A nucleic acid, wherein the first strand includes the following modification pattern:

M^{A}-(M)₅-M^{B}.

A nucleic acid wherein the first strand includes the following modification pattern:

F-(Me)₃-F.

A nucleic acid wherein the first strand includes the following modification pattern:

F-(Me)₄-F.

A nucleic acid, wherein the first strand includes the following modification pattern:

F-(Me)₅-F.

A nucleic acid, wherein the first strand includes the following modification pattern:

M^{C}-M^{A}-(M)₃₋₅-M^{B}-M^{D}

wherein M^{C} and M^{D}, which may be the same or different, respectively denote a plurality of 5′ and 3′ terminal region chemically modified nucleosides each comprising at least two differently modified nucleosides.

A nucleic acid wherein the first strand includes the following modification pattern:
3′ Me-Me-Me-Me-Me-Me-Me-F-Me-F-Me-Me-Me-Me-F—F-Me-F-Me-Me-Me-F-Me 5′.

A nucleic acid wherein the first strand includes the following modification pattern:
3′ H—H-Me-Me-Me-Me-Me-F-Me-F-Me-F-Me-Me-Me-F-Me-F-Me-Me-F-Me-F—F-Me 5′.

A nucleic acid wherein the first strand includes the following modification pattern:
3′ Me-Me-Me-Me-Me-Me-Me-F-Me-F-Me-Me-Me-Me-F-Me-Me-F-Me-Me-Me-F-Me 5′.

A nucleic acid wherein the second strand includes the following modification pattern:
ia-ia-Me-Me-Me-Me-Me-Me-F—F—F—F—F-Me-Me-Me-Me-Me-Me-Me-F-Me-Me, or
ia-ia-Me-Me-Me-Me-Me-F—F-Me-F—F—F—F-Me-Me-Me-Me-Me-Me-Me-Me-Me, or
ia-ia-Me-Me-Me-Me-Me-Me-F-Me-F—F—F—F-Me-Me-Me-Me-Me-Me-Me-Me-Me, or
ia-ia-Me-Me-Me-Me-Me-Me-Me-Me-F—F—F-Me-Me-Me-Me-Me-Me-Me-Me-Me-Me, or
ia-ia-Me-Me-Me-Me-Me-Me-F-Me-F—F—F-Me-Me-Me-Me-Me-Me-Me-Me-Me-Me, or
Me-Me-Me-Me-Me-Me-F—F—F—F—F-Me-Me-Me-Me-Me-Me-Me-F-Me-Me-ia-ia, or
Me-Me-Me-Me-Me-F—F-Me-F—F—F—F-Me-Me-Me-Me-Me-Me-Me-Me-Me-ia-ia, or
Me-Me-Me-Me-Me-Me-F-Me-F—F—F—F-Me-Me-Me-Me-Me-Me-Me-Me-Me-ia-ia, or
Me-Me-Me-Me-Me-Me-Me-Me-F—F—F-Me-Me-Me-Me-Me-Me-Me-Me-Me-Me-ia-ia, or
Me-Me-Me-Me-Me-Me-F-Me-F—F—F-Me-Me-Me-Me-Me-Me-Me-Me-Me-Me-ia-ia.

A nucleic acid wherein the modified nucleosides have the following modification patterns:

### Modification Pattern 1:

Second strand (5′-3′): ia-ia-Me-Me-Me-Me-Me-Me-F—F—F—F—F-Me-Me-Me-Me-Me-Me-Me-F-Me-Me,
First strand (5′-3′): Me-F-Me-F-Me-F-Me-F—F-Me-Me-Me-Me-F-Me-F-Me-e-Me-Me-Me-Me-Me,

### Or Modification Pattern 2:

Second strand (5′-3′): ia-ia-Me-Me-Me-Me-Me-F—F-Me-F—F—F—F-Me-Me-Me-Me-Me-Me-Me-Me-Me,
First strand (5′-3′): Me-F-Me-F-Me-F-Me-F—F-Me-Me-Me-Me-F-Me-F-Me-Me-Me-Me-Me-Me-Me,

### Or Modification Pattern 3:

Second strand (5′-3′): ia-ia-Me-Me-Me-Me-Me-Me-F-Me-F—F—F—F-Me-Me-Me-Me-Me-Me-Me-Me-Me,
First strand (5′-3′): Me-F-Me-F-Me-F-Me-F—F-Me-Me-Me-Me-F-Me-F-Me-Me-Me-Me-Me-Me-Me,

### Or Modification Pattern 4:

Second strand (5′-3′): ia-ia-Me-Me-Me-Me-Me-Me-F-Me-F—F—F—F-Me-Me-Me-Me-Me-Me-Me-Me-Me,
First strand (5′-3′): Me-F-Me-F-Me-F-Me-Me-Me-Me-Me-Me-Me-F-Me-F-Me-Me-Me-Me-Me-Me-Me,

### Or Modification Pattern 5:

Second strand (5′-3′): ia-ia-Me-Me-Me-Me-Me-Me-Me-Me-F—F—F-Me-Me-Me-Me-Me-Me-Me-Me-Me-Me,
First strand (5′-3′): Me-F-Me-Me-Me-F-Me-Me-F-Me-Me-Me-Me-F-Me-F-Me-Me-Me-Me-Me-Me-Me,

### Or Modification Pattern 6:

Second strand (5′-3′): ia-ia-Me-Me-Me-Me-Me-Me-F-Me-F—F—F-Me-Me-Me-Me-Me-Me-Me-Me-Me-Me,
First strand (5′-3′): Me-F-Me-Me-Me-F-Me-Me-F-Me-Me-Me-Me-F-Me-F-Me-Me-Me-Me-Me-Me-Me,

### Or Modification Pattern 7:

Second strand (5′-3′): Me-Me-Me-Me-Me-Me-F—F—F—F—F-Me-Me-Me-Me-Me-Me-Me-F-Me-Me-ia-ia,
First strand (5′-3′): Me-F-Me-F-Me-F-Me-F—F-Me-Me-Me-Me-F-Me-F-Me-Me-Me-Me-Me-Me-Me,

### Or Modification Pattern 8:

Second strand (5′-3′): Me-Me-Me-Me-Me-F—F-Me-F—F—F—F-Me-Me-Me-Me-Me-Me-Me-Me-Me-ia-ia,
First strand (5′-3′): Me-F-Me-F-Me-F-Me-F—F-Me-Me-Me-Me-F-Me-F-Me-Me-Me-Me-Me-Me-Me,

### Or Modification Pattern 9:

Second strand (5′-3′): Me-Me-Me-Me-Me-Me-F-Me-F—F—F—F-Me-Me-Me-Me-Me-Me-Me-Me-Me-ia-ia,
First strand (5′-3′): Me-F-Me-F-Me-F-Me-F—F-Me-Me-Me-Me-F-Me-F-Me-Me-Me-Me-Me-Me-Me,

### Or Modification Pattern 10:

Second strand (5′-3′): Me-Me-Me-Me-Me-Me-F-Me-F—F—F—F-Me-Me-Me-Me-Me-Me-Me-Me-Me-ia-ia,
First strand (5′-3′): Me-F-Me-F-Me-F-Me-Me-Me-Me-Me-Me-Me-F-Me-F-Me-Me-Me-Me-Me-Me-Me,

### Or Modification Pattern 11:

Second strand (5′-3′): Me-Me-Me-Me-Me-Me-Me-Me-F—F—F-Me-Me-Me-Me-Me
Me-Me-Me-Me-Me-ia-ia,
First strand (5′-3′): Me-F-Me-Me-Me-F-Me-Me-F-Me-Me-Me-Me-F-Me-F-Me-Me-Me-Me-Me-Me-Me,

### Or Modification Pattern 12:

Second strand (5′-3′): Me-Me-Me-Me-Me-Me-F-Me-F—F—F-Me-Me-Me-Me-Me-Me-Me-Me-Me-Me-ia-ia,
First strand (5′-3′): Me-F-Me-Me-Me-F-Me-Me-F-Me-Me-Me-Me-F-Me-F-Me-Me-Me-Me-Me-Me-Me.

A nucleic acid which is an siRNA oligonucleoside, wherein each of the first and second strands comprises an alternating modification pattern, preferably a fully alternating modification pattern along the entire length of each of the first and second strands, wherein the nucleosides of the first strand are modified by (i) 2′Me modifications on the odd numbered nucleosides counting from position 1 of the first strand, and (ii) 2′F modifications on the even numbered nucleosides counting from position 1 of the first strand, and nucleosides of the second strand are modified by (i) 2′F modifications on the odd numbered nucleosides counting from position 1 of the second strand, and (ii) 2′Me modifications on the even numbered nucleosides counting from position 1 of the second strand. Typically such fully alternating modification patterns are present in a blunt ended oligonucleoside, wherein each of the first and second strands are 19 nucleosides in length.

Position 1 of the first or the second strand is the nucleoside which is the closest to the end of the nucleic acid (ignoring any abasic nucleosides) and that is joined to an adjacent nucleoside (at Position 2) via a 3′ to 5′ internal bond, with reference to the bonds between the sugar moieties of the backbone, and reading in a direction away from that end of the molecule.

It can therefore be seen that “position 1 of the sense strand” is the 5′ most nucleoside (not including abasic nucleosides) at the conventional 5′ end of the sense strand. Typically, the nucleoside at this position 1 of the sense strand will be equivalent to the 5′ nucleoside of the selected target nucleic acid sequence, and more generally the sense strand will have equivalent nucleosides to those of the target nucleic acid sequence starting from this position 1 of the sense strand, whilst also allowing for acceptable mismatches between the sequences.

As used herein, “position 1 of the antisense strand” is the 5′ most nucleoside (not including abasic nucleosides) at the conventional 5′ end of the antisense strand. As hereinbefore described, there will be a region of complementarity between the sense and antisense strands, and in this way the antisense strand will also have a region of complementarity to the target nucleic acid sequence as referred to above.

In certain embodiments, the nucleic acid e.g. RNAi agent further comprises at least one phosphorothioate or methylphosphonate internucleoside linkage. For example the phosphorothioate or methylphosphonate internucleoside linkage can be at the 3′-terminus or in the terminal region of one strand, i.e., the sense strand or the antisense strand; or at the ends of both strands, the sense strand and the antisense strand.

In certain embodiments, the phosphorothioate or methylphosphonate internucleoside linkage is at the 5′terminus or in the terminal region of one strand, i.e., the sense strand or the antisense strand; or at the ends of both strands, the sense strand and the antisense strand.

In certain embodiments, a phosphorothioate or a methylphosphonate internucleoside linkage is at both the 5′- and 3′-terminus or in the terminal region of one strand, i.e., the sense strand or the antisense strand; or at the ends of both strands, the sense strand and the antisense strand.

Any nucleic acid may comprise one or more phosphorothioate (PS) modifications within the nucleic acid, such as at least two PS internucleoside bonds at the ends of a strand.

At least one of the oligoribonucleoside strands preferably comprises at least two consecutive phosphorothioate modifications in the last 3 nucleosides of the oligonucleoside.

The invention therefore also relates to: A nucleic acid disclosed herein which comprises phosphorothioate internucleoside linkages respectively between at least two or three consecutive positions, such as in a 5′ and/or 3′ terminal region and/or near terminal region of the second strand, whereby said near terminal region is preferably adjacent said terminal region wherein said one or more abasic nucleosides of said second strand is/are located.

A nucleic acid disclosed herein which comprises phosphorothioate internucleoside linkages respectively between at least two or three consecutive positions in a 5′ and/or 3′ terminal region of the first strand, whereby preferably the terminal position at the 5′ and/or 3′ terminal region of said first strand is attached to its adjacent position by a phosphorothioate internucleoside linkage.

The nucleic acid strand may be an RNA comprising a phosphorothioate internucleoside linkage between the three nucleosides contiguous with 2 terminally located abasic nucleosides.

A preferred nucleic acid is a double stranded RNA comprising 2 adjacent abasic nucleosides at the 5′ terminus of the second strand and a ligand moiety comprising one or more GalNAc ligand moieties at the opposite 3′ end of the second strand. Further preferred, the same nucleic acid may also comprise a phosphorothioate bond between nucleotides at positions 3-4 and 4-5 of the second strand, reading from the position 1 of the second strand. Further preferred, the same nucleic acid may also comprise a 2′ F modification at positions 7, 9 and 11 of the second strand.

### Conjugation

Another modification of the nucleic acid e.g. RNA e.g. an iRNA of the invention involves linking the nucleic acid e.g. the iRNA to one or more ligand moieties e.g. to enhance the activity, cellular distribution, or cellular uptake of the nucleic acid e.g. iRNA e.g., into a cell.

In some embodiments, the ligand moiety described can be attached to a nucleic acid e.g. an iRNA oligonucleoside, via a linker that can be cleavable or non-cleavable. The term “linker” or “linking group” means an organic moiety that connects two parts of a compound, e.g., covalently attaches two parts of a compound.

The ligand can be attached to the 3′ or 5′ end of the sense strand.

The ligand is preferably conjugated to 3′ end of the sense strand of the nucleic acid e.g. an RNAi agent.

The invention therefore relates in a further aspect to a conjugate for inhibiting expression of a target gene in a cell, said conjugate comprising a nucleic acid portion and one or more ligand moieties, said nucleic acid portion comprising a nucleic acid as disclosed herein.

In one aspect the second strand of the nucleic acid is conjugated directly or indirectly (e.g. via a linker) to the one or more ligand moiety(s), wherein said ligand moiety is typically present at a terminal region of the second strand, preferably at the 3′ terminal region thereof.

In certain embodiments, the ligand moiety comprises a GalNAc or GalNAc derivative attached to the nucleic acid eg dsRNA through a linker.

Therefore the invention relates to a conjugate wherein the ligand moiety comprises
i) one or more GalNAc ligands; and/or
ii) one or more GalNAc ligand derivatives; and/or
iii) one or more GalNAc ligands conjugated to said nucleic acid through a linker.

Said GalNAc ligand may be conjugated directly or indirectly to the 5′ or 3′ terminal region of the second strand of the nucleic acid, preferably at the 3′ terminal region thereof.

GalNAc ligands are well known in the art and described in, inter alia, EP3775207A1.

### Vector and Cell

In one aspect, the invention provides a cell containing a nucleic acid, such as inhibitory RNA [RNAi] as described herein.

In one aspect, the invention provides a cell comprising a vector as described herein.

### Pharmaceutically Acceptable Compositions

In one aspect, the invention provides a pharmaceutical composition for inhibiting expression of a target gene, the composition comprising a nucleic acid as disclosed herein.

The pharmaceutically acceptable composition may comprise an excipient and or carrier.

Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or poly anhydrides; (22) bulking agents, such as polypeptides and amino acids (23) serum component, such as serum albumin, HDL and LDL; and (22) other non-toxic compatible substances employed in pharmaceutical formulations.

Typical pharmaceutical carriers include, but are not limited to, binding agents (e.g., pregelatinized maize starch, polyvinylpyrrolidone or hydroxypropyl methylcellulose, etc.); fillers (e.g., lactose and other sugars, microcrystalline cellulose, pectin, gelatin, calcium sulfate, ethyl cellulose, polyacrylates or calcium hydrogen phosphate, etc.); lubricants (e.g., magnesium stearate, tale, silica, colloidal silicon dioxide, stearic acid, metallic stearates, hydrogenated vegetable oils, corn starch, polyethylene glycols, sodium benzoate, sodium acetate, etc.); disintegrants (e.g., starch, sodium starch glycolate, etc.); and wetting agents (e.g., sodium lauryl sulphate, etc).

Pharmaceutically acceptable organic or inorganic excipients suitable for non-parenteral administration which do not deleteriously react with nucleic acids can also be used to formulate the compositions of the present invention. Suitable pharmaceutically acceptable excipients include, but are not limited to, water, salt solutions, alcohols, polyethylene glycols, gelatin, lactose, amylose, magnesium stearate, talc, silicic acid, viscous paraffin, hydroxymethylcellulose, polyvinylpyrrolidone, and the like.

Formulations for topical administration of nucleic acids can include sterile and non-sterile aqueous solutions, non-aqueous solutions in common solvents such as alcohols, or solutions of the nucleic acids in liquid or solid oil bases. The solutions can also contain buffers, diluents and other suitable additives. Pharmaceutically acceptable organic or inorganic excipients suitable for non-parenteral administration which do not deleteriously react with nucleic acids can be used.

In one embodiment, the nucleic acid or composition is administered in an unbuffered solution. In certain embodiments, the unbuffered solution is saline or water. In other embodiments, the nucleic acid e.g. RNAi agent is administered in a buffered solution. In such embodiments, the buffer solution can comprise acetate, citrate, prolamine, carbonate, or phosphate, or any combination thereof. For example, the buffer solution can be phosphate buffered saline (PBS).

### Dosages

The pharmaceutical compositions of the invention may be administered in dosages sufficient to inhibit expression of a gene. In general, a suitable dose of a nucleic acid e.g. an iRNA of the invention will be in the range of about 0.001 to about 200.0 milligrams per kilogram body weight of the recipient per day, generally in the range of about 1 to 50 mg per kilogram body weight per day. Typically, a suitable dose of a nucleic acid e.g. an iRNA of the invention will be in the range of about 0.1 mg/kg to about 5.0 mg/kg, e.g., about 0.3 mg/kg and about 3.0 mg/kg.

A repeat-dose regimen may include administration of a therapeutic amount of a nucleic acid e.g. iRNA on a regular basis, such as every other day or once a year. In certain embodiments, the nucleic acid e.g. iRNA is administered about once per month to about once per quarter (i.e., about once every three months).

In various embodiments, the nucleic acid e.g. RNAi agent is administered at a dose of about 0.01 mg/kg to about 10 mg/kg or about 0.5 mg/kg to about 50 mg/kg. In some embodiments, the nucleic acid e.g. RNAi agent is administered at a dose of about 10 mg/kg to about 30 mg/kg. In certain embodiments, the nucleic acid e.g. RNAi agent is administered at a dose selected from about 0.5 mg/kg 1 mg/kg, 1.5 mg/kg, 3 mg/kg, 5 mg/kg, 10 mg/kg, and 30 mg/kg. In certain embodiments, the nucleic acid e.g. RNAi agent is administered about once per week, once per month, once every other two months, or once a quarter (i.e., once every three months) at a dose of about 0.1 mg/kg to about 5.0 mg/kg. In certain embodiments, the nucleic acid e.g. RNAi agent is administered to the subject once a week. In certain embodiments, the nucleic acid e.g. RNAi agent is administered to the subject once a month. In certain embodiments, the nucleic acid e.g. RNAi agent is administered once per quarter (i.e; every three months).

After an initial treatment regimen, the treatments can be administered on a less frequent basis. For example, after administration weekly or biweekly for three months, administration can be repeated once per month, for six months, or a year; or longer.

The pharmaceutical composition can be administered once daily, or administered as two, three, or more sub-doses at appropriate intervals throughout the day or even using continuous infusion or delivery through a controlled release formulation. In that case, the nucleic acid e.g. iRNA contained in each sub-dose must be correspondingly smaller in order to achieve the total daily dosage. The dosage unit can also be compounded for delivery over several days, e.g., using a conventional sustained release formulation which provides sustained release of the nucleic acid e.g. iRNA over a several day period. Sustained release formulations are well known in the art and are particularly useful for delivery of agents at a particular site, such as could be used with the agents of the present invention. In this embodiment, the dosage unit contains a corresponding multiple of the daily dose.

In other embodiments, a single dose of the pharmaceutical compositions can be long lasting, such that subsequent doses are administered at not more than 3, 4, or 5 day intervals, or at not more than 1, 2, 3, or 4 week intervals. In some embodiments of the invention, a single dose of the pharmaceutical compositions of the invention is administered once per week. In other embodiments of the invention, a single dose of the pharmaceutical compositions of the invention is administered bimonthly. In certain embodiments, the iRNA is administered about once per month to about once per quarter (i.e., about once every three months), or even every 6 months or 12 months.

Estimates of effective dosages and in vivo half-lives for the individual nucleic acid e.g. iRNAs encompassed by the invention can be made using conventional methodologies or on the basis of in vivo testing using an appropriate animal model, as known in the art.

The pharmaceutical compositions of the present invention can be administered in a number of ways depending upon whether local or systemic treatment is desired and upon the area to be treated. Administration can be topical {e.g., by a transdermal patch), pulmonary, e.g., by inhalation or insufflation of powders or aerosols, including by nebulizer; intratracheal, intranasal, epidermal and transdermal, oral or parenteral. Parenteral administration includes intravenous, intraarterial, subcutaneous, intraperitoneal, or intramuscular injection or infusion; subdermal, e.g., via an implanted device; or intracranial, e.g., by intraparenchymal, intrathecal or intraventricular administration. In certain preferred embodiments, the compositions are administered by intravenous infusion or injection. In certain embodiments, the compositions are administered by subcutaneous injection.

In one embodiment, the nucleic acid e.g. RNAi agent is administered to the subject subcutaneously.

The nucleic acid e.g. iRNA can be delivered in a manner to target a particular tissue {e.g. in particular liver cells).

### Methods for Inhibiting Gene Expression

The present invention also provides methods of inhibiting expression of a gene in a cell. The methods include contacting a cell with an nucleic acid of the invention e.g. RNAi agent, such as double stranded RNAi agent, in an amount effective to inhibit expression of the gene in the cell, thereby inhibiting expression of the gene in the cell.

Contacting of a cell with the nucleic acid e.g. an iRNA, such as a double stranded RNAi agent, may be done in vitro or in vivo. Contacting a cell in vivo with nucleic acid e.g. iRNA includes contacting a cell or group of cells within a subject, e.g., a human subject, with the nucleic acid e.g. iRNA. Combinations of in vitro and in vivo methods of contacting a cell are also possible. Contacting a cell may be direct or indirect, as discussed above. Furthermore, contacting a cell may be accomplished via a targeting ligand moiety, including any ligand moiety described herein or known in the art. In preferred embodiments, the targeting ligand moiety is a carbohydrate moiety, e.g. a GalNAc3 ligand, or any other ligand moiety that directs the RNAi agent to a site of interest.

The term “inhibiting,” as used herein, is used interchangeably with “reducing,” “silencing,” “downregulating”, “suppressing”, and other similar terms, and includes any level of inhibition.

In some embodiments of the methods of the invention, expression of a gene is inhibited by at least 30%, 35%, 40%, 45%, 50%, 55%, 60%, 65%, 70%, 75%, 80%, 85%, 90%, or 95%, or to below the level of detection of the assay. In certain embodiments, the methods include a clinically relevant inhibition of expression of a target gene e.g. as demonstrated by a clinically relevant outcome after treatment of a subject with an agent to reduce the expression of the gene

Inhibition of the expression of a gene may be manifested by a reduction of the amount of mRNA of the target gene of interest in comparison to a suitable control.

In other embodiments, inhibition of the expression of a gene may be assessed in terms of a reduction of a parameter that is functionally linked to gene expression, e.g, protein expression or signalling pathways.

Methods of Treating or Preventing Diseases Associated with Gene Expression

The present invention also provides methods of using nucleic acid e.g. an iRNA of the invention or a composition containing nucleic acid e.g. an iRNA of the invention to reduce or inhibit gene expression in a cell. The methods include contacting the cell with a nucleic acid e.g. dsRNA of the invention and maintaining the cell for a time sufficient to obtain degradation of the mRNA transcript of a gene, thereby inhibiting expression of the gene in the cell. Reduction in gene expression can be assessed by any methods known in the art.

In the methods of the invention the cell may be contacted in vitro or in vivo, i.e., the cell may be within a subject.

A cell suitable for treatment using the methods of the invention may be any cell that expresses a gene of interest associated with disease.

The in vivo methods of the invention may include administering to a subject a composition containing a nucleic acid of the invention e.g. an iRNA, where the nucleic acid e.g. iRNA includes a nucleoside sequence that is complementary to at least a part of an RNA transcript of the gene of the mammal to be treated.

The present invention further provides methods of treatment of a subject in need thereof. The treatment methods of the invention include administering a nucleic acid such as an iRNA of the invention to a subject, e.g., a subject that would benefit from a reduction or inhibition of the expression of a gene, in a therapeutically effective amount e.g. a nucleic acid such as an iRNA targeting a gene or a pharmaceutical composition comprising the nucleic acid targeting a gene.

An nucleic acid e.g. iRNA of the invention may be administered as a “free” nucleic acid or “free iRNA, administered in the absence of a pharmaceutical composition. The naked nucleic acid may be in a suitable buffer solution. The buffer solution may comprise acetate, citrate, prolamine, carbonate, or phosphate, or any combination thereof. In one embodiment, the buffer solution is phosphate buffered saline (PBS). The pH and osmolarity of the buffer solution can be adjusted such that it is suitable for administering to a subject.

Alternatively, a nucleic acid e.g. iRNA of the invention may be administered as a pharmaceutical composition, such as a dsRNA liposomal formulation.

In one embodiment, the method includes administering a composition featured herein such that expression of the target gene is decreased, such as for about 1, 2, 3, 4, 5, 6, 7, 8, 12, 16, 18, 24 hours, 28, 32, or about 36 hours. In one embodiment, expression of the target gene is decreased for an extended duration, e.g., at least about two, three, four days or more, e.g., about one week, two weeks, three weeks, or four weeks or longer, e.g., about 1 month, 2 months, or 3 months.

Subjects can be administered a therapeutic amount of nucleic acid e.g. iRNA, such as about 0.01 mg/kg to about 200 mg/kg.

The nucleic acid e.g. iRNA can be administered by intravenous infusion over a period of time, on a regular basis. In certain embodiments, after an initial treatment regimen, the treatments can be administered on a less frequent basis. Administration of the iRNA can reduce gene product levels of a target gene, e.g., in a cell or tissue of the patient by at least about 20%, 25%, 30%, 35%, 40%, 45%, 50%, 55%, 60%, 65%, 70%, 75%, 80%, 85%, 90%, or 95%, or below the level of detection of the assay method used. In certain embodiments, administration results in clinical stabilization or preferably clinically relevant reduction of at least one sign or symptom of a gene-associated disorder.

Alternatively, the nucleic acid e.g. iRNA can be administered subcutaneously, i.e., by subcutaneous injection. One or more injections may be used to deliver the desired daily dose of nucleic acid e.g. iRNA to a subject. The injections may be repeated over a period of time. The administration may be repeated on a regular basis. In certain embodiments, after an initial treatment regimen, the treatments can be administered on a less frequent basis. A repeat-dose regimen may include administration of a therapeutic amount of nucleic acid on a regular basis, such as every other day or to once a year. In certain embodiments, the nucleic acid is administered about once per month to about once per quarter (i.e. about once every three months).

In one aspect the present invention may be applied in the compounds, processes, compositions or uses of the following Sentences numbered 1-101 (wherein reference to any Formula in the Sentences 1-101 refers only to those Formulas that are defined within Sentences 1-101. These formulae are reproduced in FIG. 43)
1. A compound comprising the following structure:

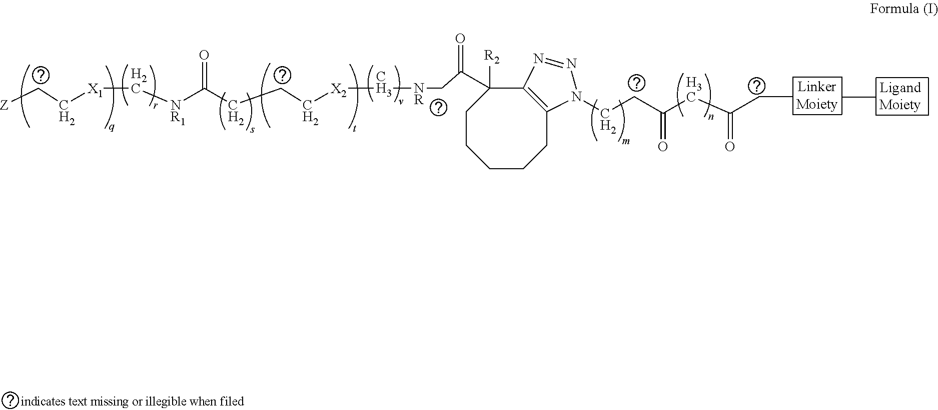

wherein:
R₁ at each occurrence is independently selected from the group consisting of hydrogen, methyl and ethyl;
R₂ is selected from the group consisting of hydrogen, hydroxy, —OC₁₋₃alkyl, —C(═O)OC₁₋₃alkyl, halo and nitro;
X₁ and X₂ at each occurrence are independently selected from the group consisting of methylene, oxygen and sulfur;
m is an integer of from 1 to 6;
n is an integer of from 1 to 10;
q, r, s, t, v are independently integers from 0 to 4, with the proviso that:
(i) q and r cannot both be 0 at the same time; and
(ii) s, t and v cannot all be 0 at the same time;
Z is an oligonucleoside moiety.
2. A compound according to Sentence 1, wherein R1 is hydrogen at each occurrence.
3. A compound according to Sentence 1, wherein R1 is methyl.
4. A compound according to Sentence 1, wherein R1 is ethyl.
5. A compound according to any of Sentences 1 to 4, wherein R2 is hydroxy.
6. A compound according to any of Sentences 1 to 4, wherein R2 is halo.
7. A compound according to Sentence 6, wherein R2 is fluoro.
8. A compound according to Sentence 6, wherein R2 is chloro.
9. A compound according to Sentence 6, wherein R2 is bromo.
10. A compound according to Sentence 6, wherein R2 is iodo.
11. A compound according to Sentence 6, wherein R₂ is nitro.
12. A compound according to any of Sentences 1 to 11, wherein X1 is methylene.
13. A compound according to any of Sentences 1 to 11, wherein X1 is oxygen.
14. A compound according to any of Sentences 1 to 11, wherein X1 is sulfur.
15. A compound according to any of Sentences 1 to 14, wherein X2 is methylene.
16. A compound according to any of Sentences 1 to 15, wherein X2 is oxygen.
17. A compound according to any of Sentences 1 to 16, wherein X₂ is sulfur.
18. A compound according to any of Sentences 1 to 17, wherein m=3.
19. A compound according to any of Sentences 1 to 18, wherein n=6.
20. A compound according to Sentences 13 and 15, wherein X₁ is oxygen and X₂ is methylene, and preferably wherein:
   q=1,
   r=2,
   s=1,
   t=1,
   v=1.
21. A compound according to Sentences 12 and 15, wherein both X₁ and X₂ are methylene, and preferably wherein:
   q=1,
   r=3,
   s=1,
   t=1,
   v=1.
22. A compound according to any of Sentences 1 to 21, wherein Z is:

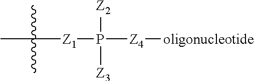

wherein:
Z1, Z2, Z3, Z4 are independently at each occurrence oxygen or sulfur; and
one the bonds between P and Z2, and P and Z3 is a single bond and the other bond is a double bond.
23. A compound according to Sentence 22, wherein said oligonucleoside is an RNA compound capable of modulating, preferably inhibiting, expression of a target gene.
24. A compound according to Sentence 23, wherein said RNA compound comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends.
25. A compound according to Sentence 24, wherein the RNA compound is attached at the 5′ end of its second strand to the adjacent phosphate.
26. A compound according to Sentence 24, wherein the RNA compound is attached at the 3′ end of its second strand to the adjacent phosphate.
27. A compound of Formula (II):

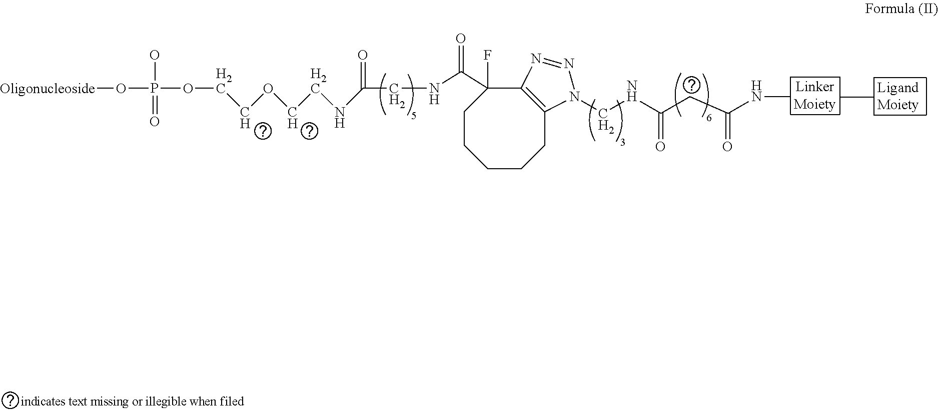

28. A compound of Formula (III):

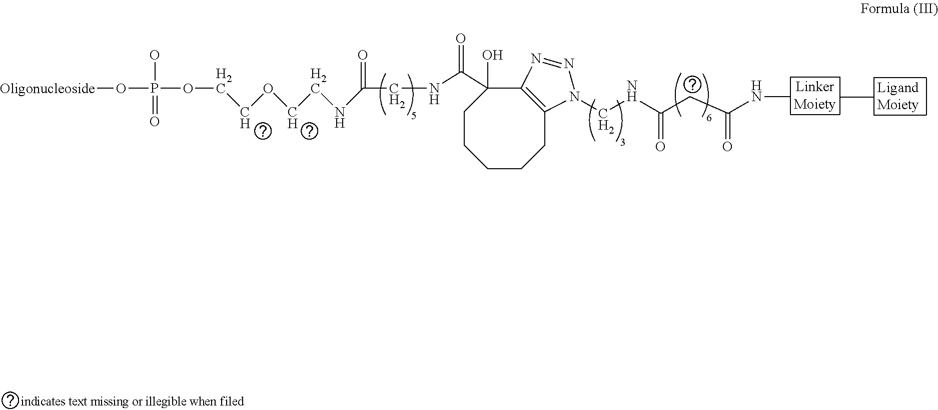

29. A compound according to Sentence 27 or 28, wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 5′ end of its second strand to the adjacent phosphate.
30. A composition comprising a compound of Formula (II) as defined in Sentence 27, and a compound of Formula (III) as defined in Sentence 28, optionally dependent on Sentence 29.
31. A composition according to Sentence 30, wherein said compound of Formula (III) as defined in Sentence 28 is present in an amount in the range of 10 to 15% by weight of said composition.
32. A compound of Formula (IV):

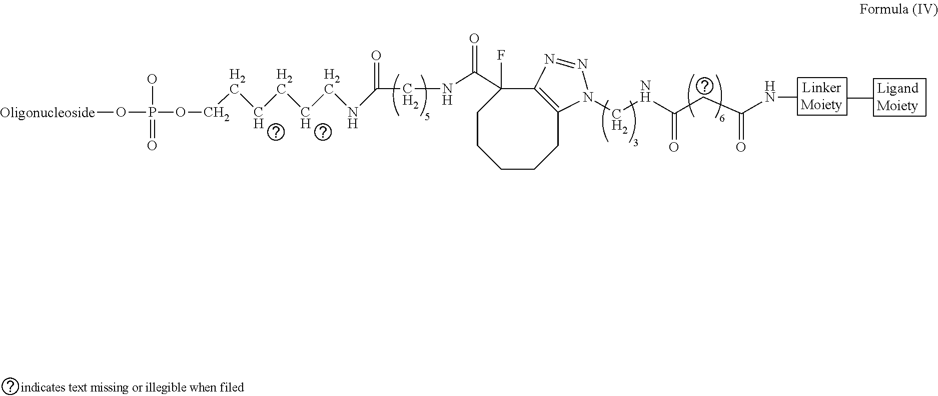

33. A compound of Formula (V):

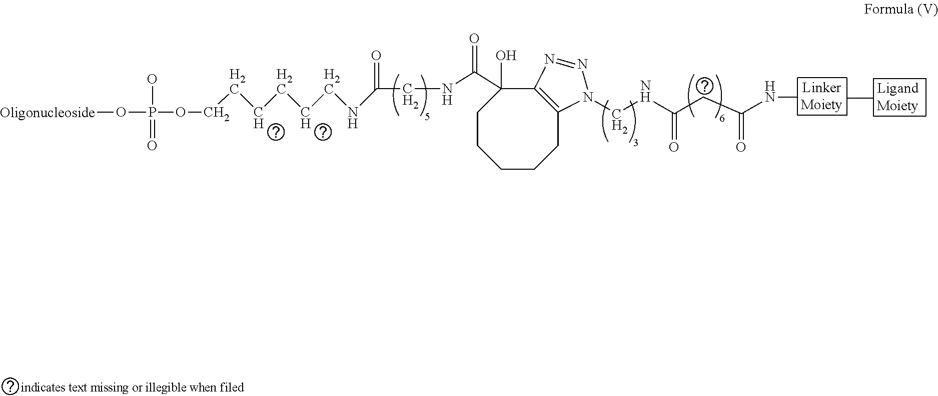

34. A compound according to Sentence 32 or 33, wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 3′ end of its second strand to the adjacent phosphate.
35. A composition comprising a compound of Formula (IV) as defined in Sentence 32, and a compound of Formula (V) as defined in Sentence 33, optionally dependent on Sentence 34.
36. A composition according to Sentence 35, wherein said compound of Formula (V) as defined in Sentence 33 is present in an amount in the range of 10 to 15% by weight of said composition.
37. A compound as defined in any of Sentences 1 to 29, or 32 to 34, wherein the oligonucleoside comprises an RNA duplex which further comprises one or more riboses modified at the 2′ position, preferably a plurality of riboses modified at the 2′ position.
38. A compound according to Sentence 37, wherein the modifications are chosen from 2′-O-methyl, 2′-deoxy-fluoro, and 2′-deoxy.
39. A compound according to any of Sentences 1 to 29, or 32 to 34, or 37 to 38, wherein the oligonucleoside further comprises one or more degradation protective moieties at one or more ends.
40. A compound according to Sentence 39, wherein said one or more degradation protective moieties are not present at the end of the oligonucleoside strand that carries the ligand moieties, and/or wherein said one or more degradation protective moieties is selected from phosphorothioate internucleoside linkages, phosphorodithioate internucleoside linkages and inverted abasic nucleosides, wherein said inverted abasic nucleosides are present at the distal end of the strand that carries the ligand moieties.
41. A compound according to any of Sentences 1 to 29, or 32 to 34, or 37 to 40, wherein said ligand moiety as depicted in Formula (I) in Sentence 1 comprises one or more ligands.
42. A compound according to Sentence 41, wherein said ligand moiety as depicted in Formula (I) in Sentence 1 comprises one or more carbohydrate ligands.
43. A compound according to Sentence 42, wherein said one or more carbohydrates can be a monosaccharide, disaccharide, trisaccharide, tetrasaccharide, oligosaccharide or polysaccharide.
44. A compound according to Sentence 43, wherein said one or more carbohydrates comprise one or more galactose moieties, one or more lactose moieties, one or more N-AcetylGalactosamine moieties, and/or one or more mannose moieties.
45. A compound according to Sentence 44, wherein said one or more carbohydrates comprise one or more N-Acetyl-Galactosamine moieties.
46. A compound according to Sentence 45, which comprises two or three N-AcetylGalactosamine moieties.
47. A compound according to any of Sentences 41 to 46, wherein said one or more ligands are attached in a linear configuration, or in a branched configuration.
48. A compound according to Sentence 47, wherein said one or more ligands are attached as a biantennary or triantennary branched configuration.
49. A compound according to Sentences 46 to 48, wherein said moiety:

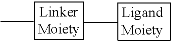

as depicted in Formula (I) in Sentence 1 is any of Formulae (VIa), (VIb) or (VIc), preferably Formula (VIa):

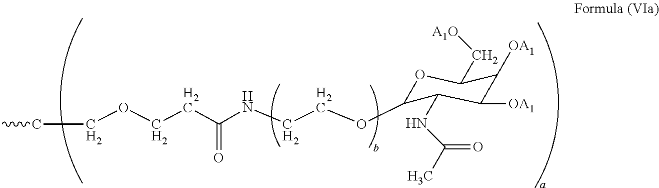

wherein:
A₁ is hydrogen, or a suitable hydroxy protecting group;
a is an integer of 2 or 3; and
b is an integer of 2 to 5; or

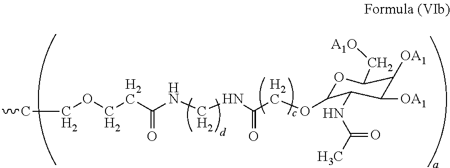

wherein:
A₁ is hydrogen, or a suitable hydroxy protecting group;
a is an integer of 2 or 3; and
c and d are independently integers of 1 to 6; or

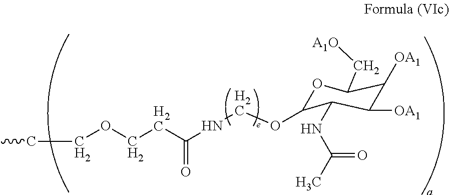

wherein:
A₁ is hydrogen, or a suitable hydroxy protecting group;
a is an integer of 2 or 3; and
e is an integer of 2 to 10.
50. A compound according to Sentences 46 to 48, wherein said moiety:

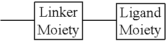

as depicted in Formula (I) in Sentence 1 is Formula (VII):

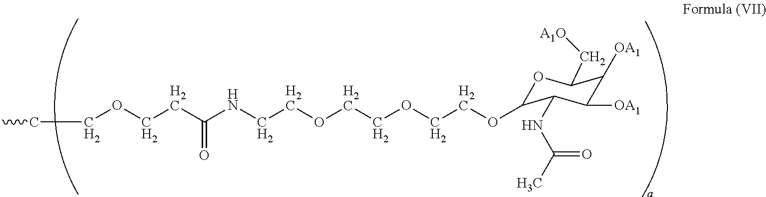

wherein:
A₁ is hydrogen;
a is an integer of 2 or 3.
51. A compound according to Sentence 49 or 50, wherein a=2.
52. A compound according to Sentence 49 or 50, wherein a=3.
53. A compound according to Sentence 49, wherein b=3.
54. A compound of Formula (VIII):

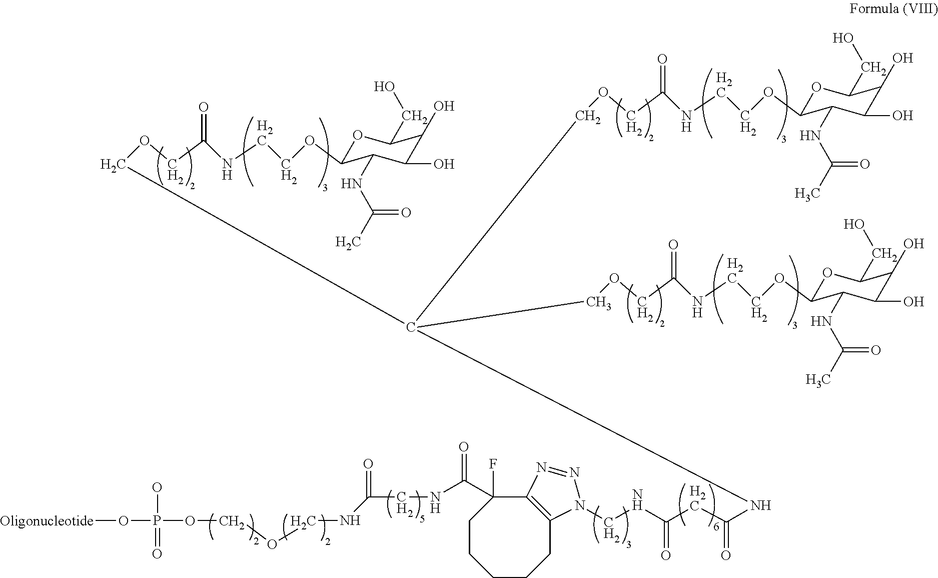

55. A compound of Formula (IX):

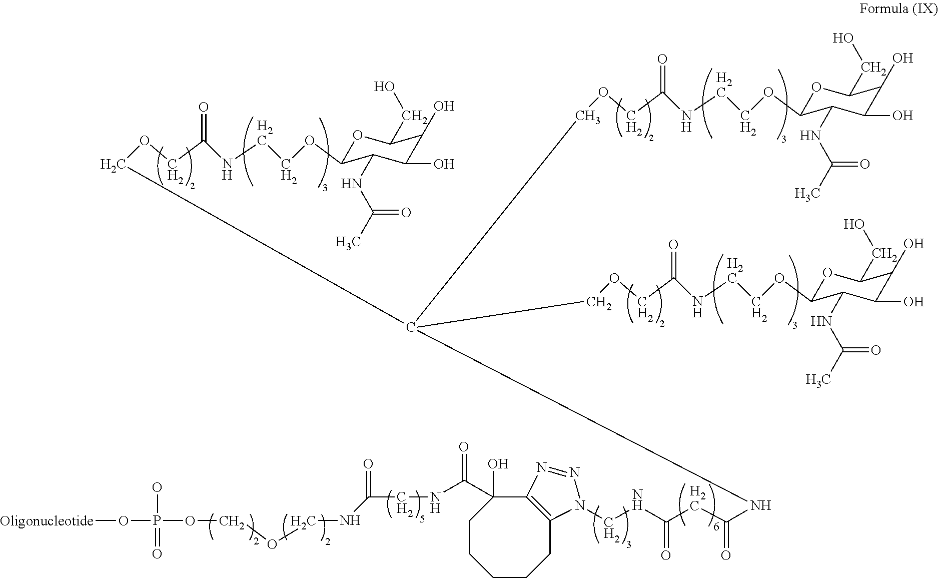

56. A compound according to Sentence 54 or 55, wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 5′ end of its second strand to the adjacent phosphate.
57. A composition comprising a compound of Formula (VIII) as defined in Sentence 54, and a compound of Formula (IX) as defined in Sentence 55, optionally dependent on Sentence 56.
58. A composition according to Sentence 57, wherein said compound of Formula (IX) as defined in Sentence 55 is present in an amount in the range of 10 to 15% by weight of said composition.
59. A compound of Formula (X):

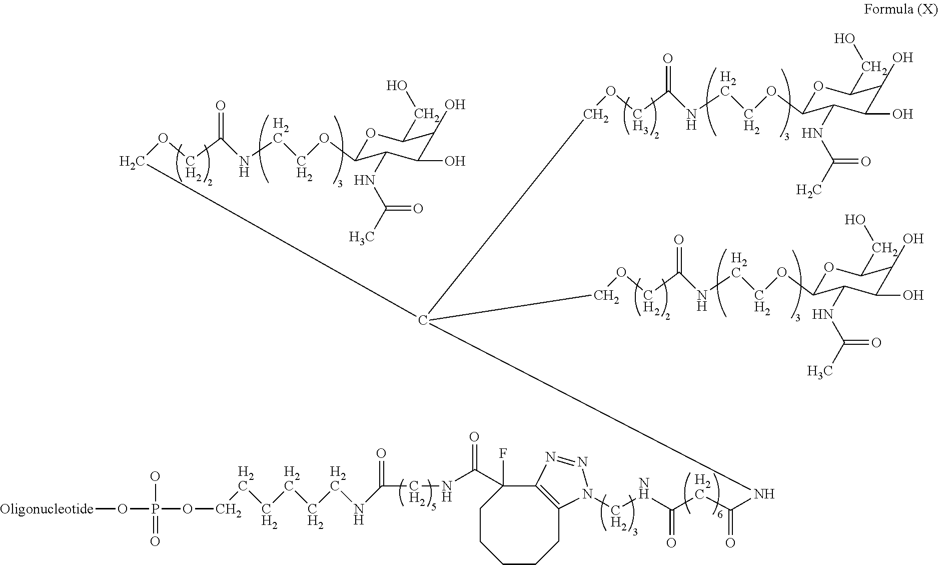

60. A compound of Formula (XI):

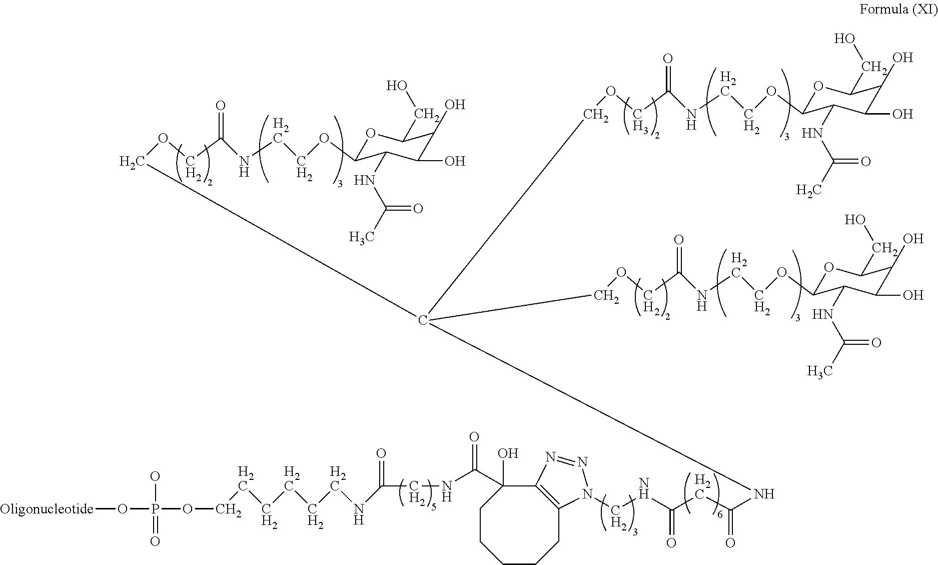

61. A compound according to Sentence 59 or 60, wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 3′ end of its second strand to the adjacent phosphate.
62. A composition comprising a compound of Formula (X) as defined in Sentence 59, and a compound of Formula (XI) as defined in Sentence 60, optionally dependent on Sentence 61.
63. A composition according to Sentence 62, wherein said compound of Formula (XI) as defined in Sentence 60 is present in an amount in the range of 10 to 15% by weight of said composition.
64. A compound as defined in any of Sentences 54 to 63, wherein the oligonucleoside comprises an RNA duplex which further comprises one or more riboses modified at the 2′ position, preferably a plurality of riboses modified at the 2′ position.
65. A compound according to Sentence 64, wherein the modifications are chosen from 2′-O-methyl, 2′-deoxy-fluoro, and 2′-deoxy.
66. A compound according to any of Sentences 54 to 65, wherein the oligonucleoside further comprises one or more degradation protective moieties at one or more ends.
67. A compound according to Sentence 66, wherein said one or more degradation protective moieties are not present at the end of the oligonucleoside strand that carries the ligand moieties, and/or wherein said one or more degradation protective moieties is selected from phosphorothioate internucleoside linkages, phosphorodithioate internucleoside linkages and inverted abasic nucleosides, wherein said inverted abasic nucleosides are present at the distal end of the strand that carries the ligand moieties, as shown in any of Formulae (VIII), (IX), (X) or (XI) in any of Sentences 54, 55, 59 or 60.
68. A process of preparing a compound according to any of Sentences 1 to 29, 32 to 34, 37 to 56, 59 to 61, and 64 to 67, and/or a composition according to any of Sentences 30, 31, 35, 36, 57, 58, 62, 63, which comprises reacting compounds of Formulae (XII) and (XIII):

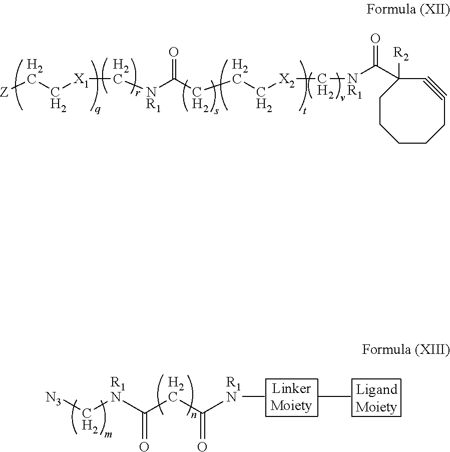

herein:
R₁ at each occurrence is independently selected from the group consisting of hydrogen, methyl and ethyl;
R₂ is selected from the group consisting of hydrogen, hydroxy, —OC₁₋₃alkyl, —C(═O)OC₁₋₃alkyl, halo and nitro;
X₁ and X2 at each occurrence are independently selected from the group consisting of methylene, oxygen and sulfur;
m is an integer of from 1 to 6;
n is an integer of from 1 to 10;
q, r, s, t, v are independently integers from 0 to 4, with the proviso that:
(i) q and r cannot both be 0 at the same time; and
(ii) s, t and v cannot all be 0 at the same time;
Z is an oligonucleoside moiety;
and where appropriate carrying out deprotection of the ligand and/or annealing of a second strand for the oligonucleoside moiety.
69. A process according to Sentence 68, wherein a compound of Formula (XII) is prepared by reacting compounds of Formulae (XIV) and (XV):

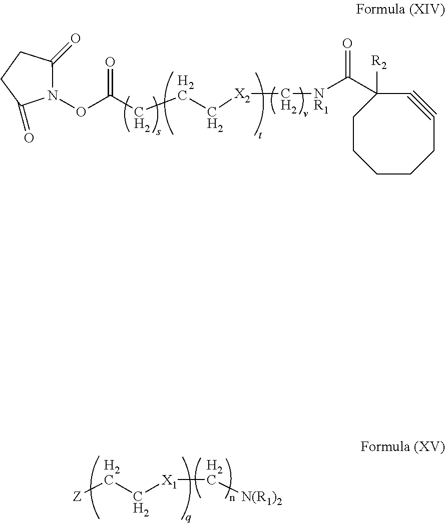

R₁ at each occurrence is independently selected from the group consisting of hydrogen, methyl and ethyl;
R₂ is selected from the group consisting of hydrogen, hydroxy, —OC₁₋₃alkyl, —C(═O)OC₁₋₃alkyl, halo and nitro;
X₁ and X₂ at each occurrence are independently selected from the group consisting of methylene, oxygen and sulfur;
q, r, s, t, v are independently integers from 0 to 4, with the proviso that:
(i) q and r cannot both be 0 at the same time; and
(ii) s, t and v cannot all be 0 at the same time;
Z is an oligonucleoside moiety.
70. A process according to Sentence 68, to prepare a compound according to any of Sentences 20, 25, 27, 29, 54, 56, and/or a composition according to any of Sentences 30, 31, 57, 58, wherein:
compound of Formula (XII) is Formula (XIIa):

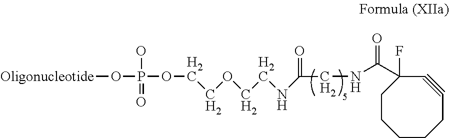

and compound of Formula (XIII) is Formula (XIIIa):

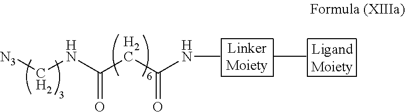

wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 5′ end of its second strand to the adjacent phosphate.
71. A process according to Sentence 68, to prepare a compound according to any of Sentences 20, 25, 28, 29, 55, 56, and/or a composition according to any of Sentences 30, 31, 57, 58, wherein: compound of Formula (XII) is Formula (XIIb):

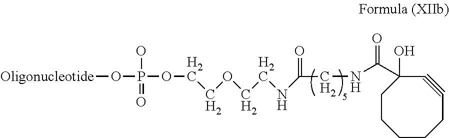

and compound of Formula (XIII) is Formula (XIIIa):

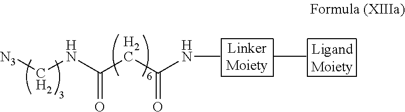

wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 5′ end of its second strand to the adjacent phosphate.
72. A process according to Sentence 68, to prepare a compound according to any of Sentences 21, 26, 32, 34, 59, 61, and/or a composition according to any of Sentences 35, 36, 62, 63, wherein:
compound of Formula (XII) is Formula (XIIc):

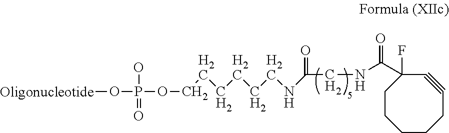

and compound of Formula (XIII) is Formula (XIIIa):

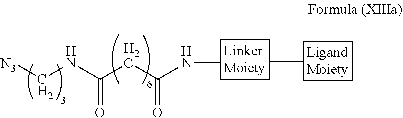

wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 3′ end of its second strand to the adjacent phosphate.
73. A process according to Sentence 68, to prepare a compound according to any of Sentences 21, 26, 33, 34, 60, 61, and/or a composition according to any of Sentences 35, 36, 62, 63, wherein:
compound of Formula (XII) is Formula (XIId):

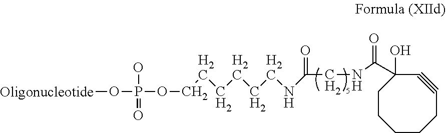

and compound of Formula (XIII) is Formula (XIIIa):

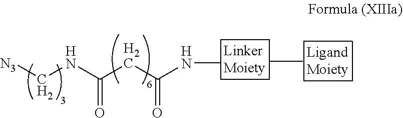

wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 3′ end of its second strand to the adjacent phosphate.
74. A process according to any of Sentences 70 to 73, wherein:
compound of Formula (XIIIa) is Formula (XIIIb):

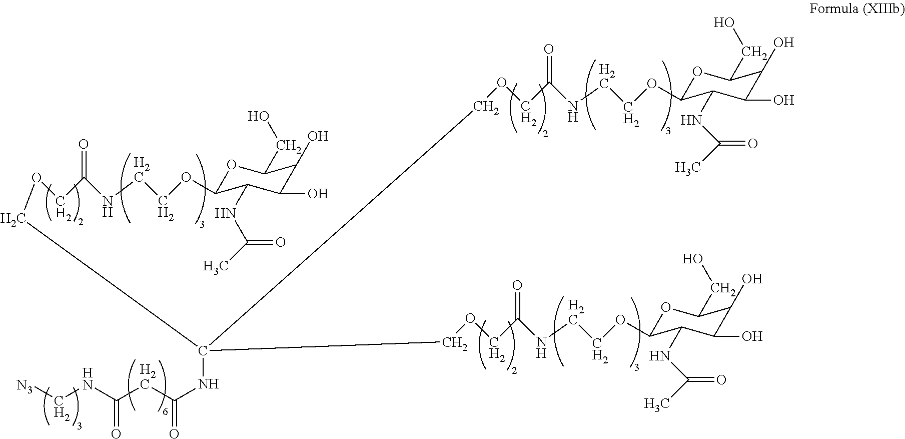

75. A process according to Sentences 69, as dependent on Sentences 70 to 73, wherein:
compound of Formula (XIV) is either Formula (XIVa) or Formula (XIVb):

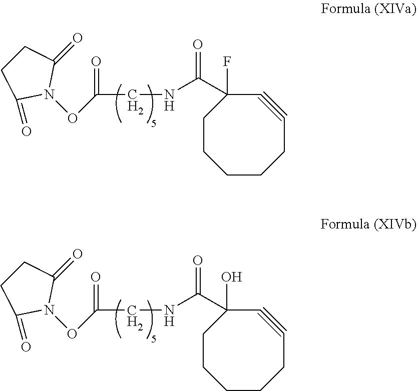

and compound of Formula (XV) is either Formula (XVa) or Formula (XIVb):

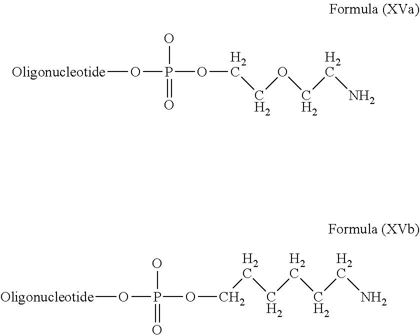

wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein (i) said RNA duplex is attached at the 5′ end of its second strand to the adjacent phosphate in Formula (XVa), or (ii) said RNA duplex is attached at the 3′ end of its second strand to the adjacent phosphate in Formula (XVb).
76. A compound of Formula (XII):

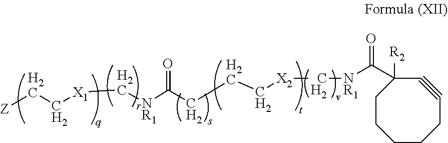

wherein:
R₁ at each occurrence is independently selected from the group consisting of hydrogen, methyl and ethyl;
R₂ is selected from the group consisting of hydrogen, hydroxy, —OC₁₋₃alkyl, —C(═O)OC₁₋₃alkyl, halo and nitro;
X₁ and X₂ at each occurrence are independently selected from the group consisting of methylene, oxygen and sulfur;
q, r, s, t, v are independently integers from 0 to 4, with the proviso that:
(i) q and r cannot both be 0 at the same time; and
(ii) s, t and v cannot all be 0 at the same time;
Z is an oligonucleoside moiety.
77. A compound of Formula (XIIa):

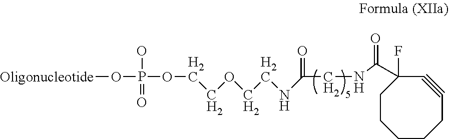

78. A compound of Formula (XIIb):

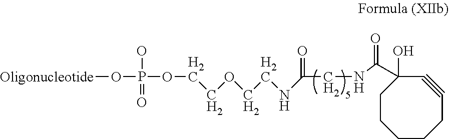

79. A compound of Formula (XIIc):

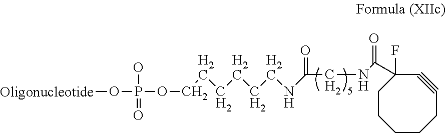

80. compound of Formula (XIId):

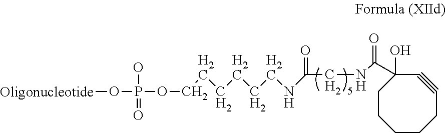

81. A compound of Formula (XIII):

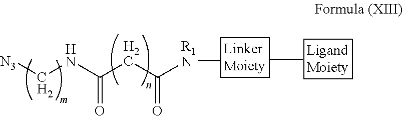

wherein:
R₁ at each occurrence is independently selected from the group consisting of hydrogen, methyl and ethyl;
m is an integer of from 1 to 6;
n is an integer of from 1 to 10.
82. A compound of Formula (XIIIa):

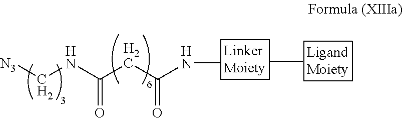

83. A compound of Formula (XIITb):

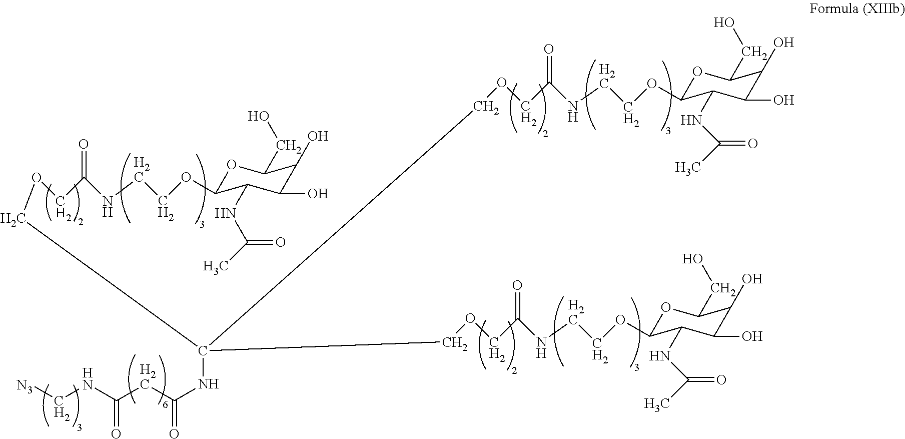

84. A compound of Formula (XIV):

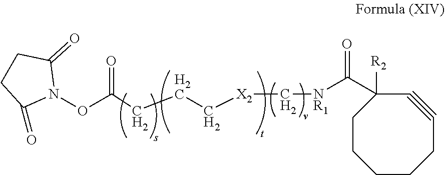

wherein:
R₁ is selected from the group consisting of hydrogen, methyl and ethyl;
R₂ is selected from the group consisting of hydrogen, hydroxy, —OC₁₋₃alkyl, —C(═O)OC₁₋₃alkyl, halo and nitro;
X₂ is selected from the group consisting of methylene, oxygen and sulfur;
s, t, y are independently integers from 0 to 4, with the proviso that s, t and y cannot all be 0 at the same time.
85. A compound of Formula (XIVa):

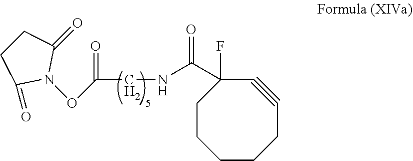

86. A compound of Formula (XIVb):

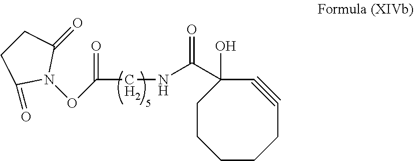

87. A compound of Formula (XV):

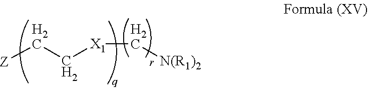

wherein:
R1 at each occurrence is independently selected from the group consisting of hydrogen, methyl and ethyl;
X1 is selected from the group consisting of methylene, oxygen and sulfur;
q and r are independently integers from 0 to 4, with the proviso that q and r cannot both be 0 at the same time;
Z is an oligonucleoside moiety.
88. A compound of Formula (XVa):

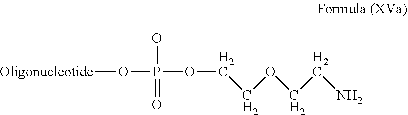

89. A compound of Formula (XVb):

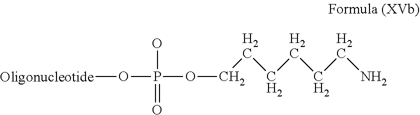

90. Use of a compound according to any of Sentences 76, 81 to 84, 87, for the preparation of a compound according to any of Sentences 1 to 29, 32 to 34, 37 to 56, 59 to 61, and 64 to 67, and/or a composition according to any of Sentences 30, 31, 35, 36, 57, 58, 62 and 63.
91. Use of a compound according to Sentence 85, for the preparation of a compound according to any of Sentences 1 to 29, 32 to 34, 37 to 56, 59 to 61, and 64 to 67, and/or a composition according to any of Sentences 30, 31, 35, 36, 57, 58, 62 and 63, wherein R2=F.
92. Use of a compound according to Sentence 86, for the preparation of a compound according to any of Sentences 1 to 29, 32 to 34, 37 to 56, 59 to 61, and 64 to 67, and/or a composition according to any of Sentences 30, 31, 35, 36, 57, 58, 62 and 63, wherein R2=OH.
93. Use of a compound according to Sentence 77, for the preparation of a compound according to any of Sentences 20, 25, 27, 29, 54, 56, and/or a composition according to any of Sentences 30, 31, 57, 58.
94. Use of a compound according to Sentence 78, for the preparation of a compound according to any of Sentences 20, 25, 28, 29, 55, 56, and/or a composition according to any of Sentences 30, 31, 57, 58.
95. Use of a compound according to Sentence 79, for the preparation of a compound according to any of Sentences 21, 26, 32, 34, 59, 61, and/or a composition according to any of Sentences 35, 36, 62, 63.
96. Use of a compound according to Sentence 80, for the preparation of a compound according to any of Sentences 21, 26, 33, 34, 60, 61, and/or a composition according to any of Sentences 35, 36, 62, 63.
97. Use of a compound according to Sentence 88, for the preparation of a compound according to any of Sentences 20, 25, 27 to 29, 54 to 56, and/or a composition according to any of Sentences 30, 31, 57, 58.
98. Use of a compound according to Sentence 89, for the preparation of a compound according to any of Sentences 21, 26, 32 to 34, 59 to 61, and/or a composition according to any of Sentences 35, 36, 62, 63.
99. A compound or composition obtained, or obtainable by a process according to any of Sentences 68 to 75.
100. A pharmaceutical composition comprising of a compound according to any of Sentences 1 to 29, 32 to 34, 37 to 56, 59 to 61, and 64 to 67, and/or a composition according to any of Sentences 30, 31, 35, 36, 57, 58, 62 and 63, together with a pharmaceutically acceptable carrier, diluent or excipient.
101. A compound according to any of Sentences 1 to 29, 32 to 34, 37 to 56, 59 to 61, and 64 to 67, and/or a composition according to any of Sentences 30, 31, 35, 36, 57, 58, 62 and 63, for use in therapy.

In another aspect the present invention may be applied in the compounds, processes, compositions or uses of the following Clauses numbered 1-56 (wherein reference to any Formula in the Clauses refers only to those Formulas that are defined within Clause 1-56. These formulae are reproduced in FIG. 44).
1. A compound comprising the following structure:

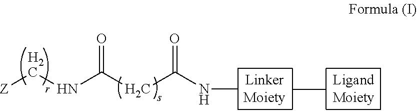

wherein:
r and s are independently an integer selected from 1 to 16; and
Z is an oligonucleoside moiety.
2. A compound according to Clause 1, wherein s is an integer selected from 4 to 12.
3. A compound according to Clause 2, wherein s is 6.
4. A compound according to any of Clauses 1 to 3, wherein r is an integer selected from 4 to 14.
5. A compound according to Clause 4, wherein r is 6.
6. A compound according to Clause 4, wherein r is 12.
7. A compound according to Clause 5, which is dependent on Clause 3.
8. A compound according to Clause 6, which is dependent on Clause 3.
9. A compound according to any of Clauses 1 to 8, wherein Z is:

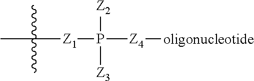

wherein:
Z₁, Z₂, Z₃, Z₄ are independently at each occurrence oxygen or sulfur; and one the bonds between P and Z₂, and P and Z₃ is a single bond and the other bond is a double bond.
10. A compound according to any of Clauses 1 to 9, wherein said oligonucleoside is an RNA compound capable of modulating, preferably inhibiting, expression of a target gene.
11. A compound according to any of Clause 10, wherein said RNA compound comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends.
12. A compound according to Clause 11, preferably also dependent on Clauses 3 and 6, wherein the RNA compound is attached at the 5′ end of its second strand to the adjacent phosphate.
13. A compound according to Clause 11, preferably also dependent on Clauses 3 and 5, wherein the RNA compound is attached at the 3′ end of its second strand to the adjacent phosphate.
14. A compound of Formula (II), preferably dependent on Clause 12:

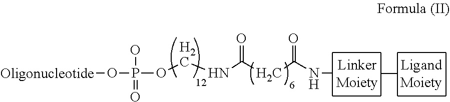

15. A compound of Formula (III), preferably dependent on Clause 13:

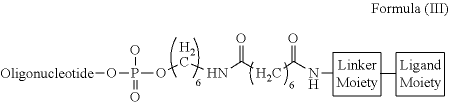

16. A compound as defined in any of Clauses 1 to 15, wherein the oligonucleoside comprises an RNA duplex which further comprises one or more riboses modified at the 2′ position, preferably a plurality of riboses modified at the 2′ position.
17. A compound according to Clause 16, wherein the modifications are chosen from 2′-O-methyl, 2′-deoxy-fluoro, and 2′-deoxy.
18. A compound according to any of Clauses 1 to 17, wherein the oligonucleoside further comprises one or more degradation protective moieties at one or more ends.
19. A compound according to Clause 18, wherein said one or more degradation protective moieties are not present at the end of the oligonucleoside strand that carries the linker/ligand moieties, and/or wherein said one or more degradation protective moieties is selected from phosphorothioate internucleoside linkages, phosphorodithioate internucleoside linkages and inverted abasic nucleosides, wherein said inverted abasic nucleosides are present at the distal end of the same strand to the end that carries the linker/ligand moieties.
20. A compound according to any of Clauses 1 to 19, wherein said ligand moiety as depicted in Formula (I) in Clause 1 comprises one or more ligands.
21. A compound according to Clause 20, wherein said ligand moiety as depicted in Formula (I) in Clause 1 comprises one or more carbohydrate ligands.
22. A compound according to Clause 21, wherein said one or more carbohydrates can be a monosaccharide, disaccharide, trisaccharide, tetrasaccharide, oligosaccharide or polysaccharide.
23. A compound according to Clause 22, wherein said one or more carbohydrates comprise one or more galactose moieties, one or more lactose moieties, one or more N-AcetylGalactosamine moieties, and/or one or more mannose moieties.
24. A compound according to Clause 23, wherein said one or more carbohydrates comprise one or more N-Acetyl-Galactosamine moieties.
25. A compound according to Clause 24, which comprises two or three N-AcetylGalactosamine moieties.
26. A compound according to any of the preceding Clauses, wherein said one or more ligands are attached in a linear configuration, or in a branched configuration.
27. A compound according to Clause 26, wherein said one or more ligands are attached as a biantennary or triantennary branched configuration.
28. A compound according to Clauses 20 to 27, wherein said moiety:

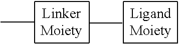

as depicted in Formula (I) in Clause 1 is any of Formulae (IV), (V) or (VI), preferably Formula (IV):

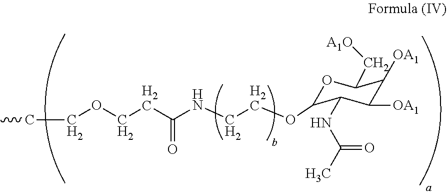

wherein:
A₁ is hydrogen, or a suitable hydroxy protecting group;
a is an integer of 2 or 3; and
b is an integer of 2 to 5; or

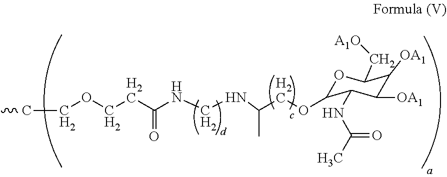

wherein:
A₁ is hydrogen, or a suitable hydroxy protecting group;
a is an integer of 2 or 3; and
c and d are independently integers of 1 to 6; or

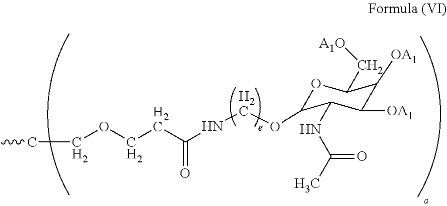

wherein:
A₁ is hydrogen, or a suitable hydroxy protecting group;
a is an integer of 2 or 3; and
e is an integer of 2 to 10.
29. A compound according to any of Clauses 1 to 28, wherein said moiety:

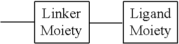

as depicted in Formula (I) in Clause 1 is Formula (VII):

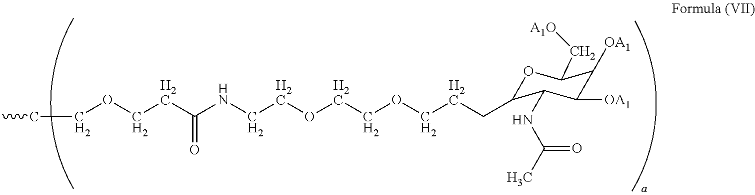

wherein:
A₁ is hydrogen;
a is an integer of 2 or 3.
30. A compound according to Clause 28 or 29, wherein a=2.
31. A compound according to Clause 28 or 29, wherein a=3.
32. A compound according to Clause 28, wherein b=3.
33. A compound of Formula (VIII):

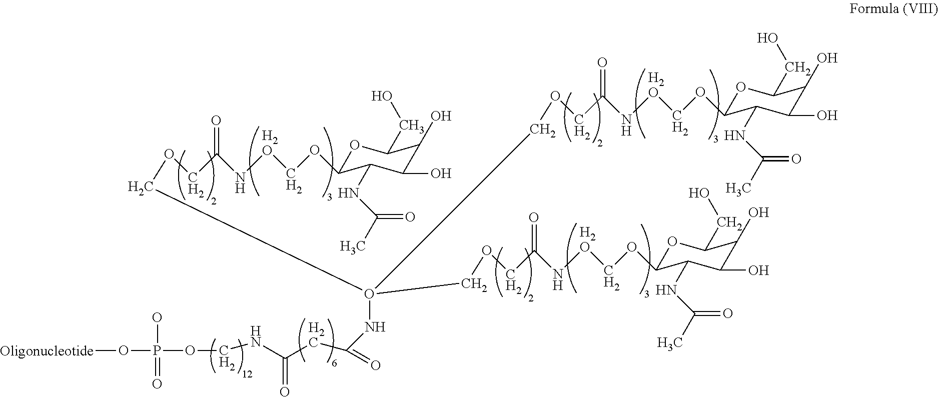

34. A compound of Formula (IX):

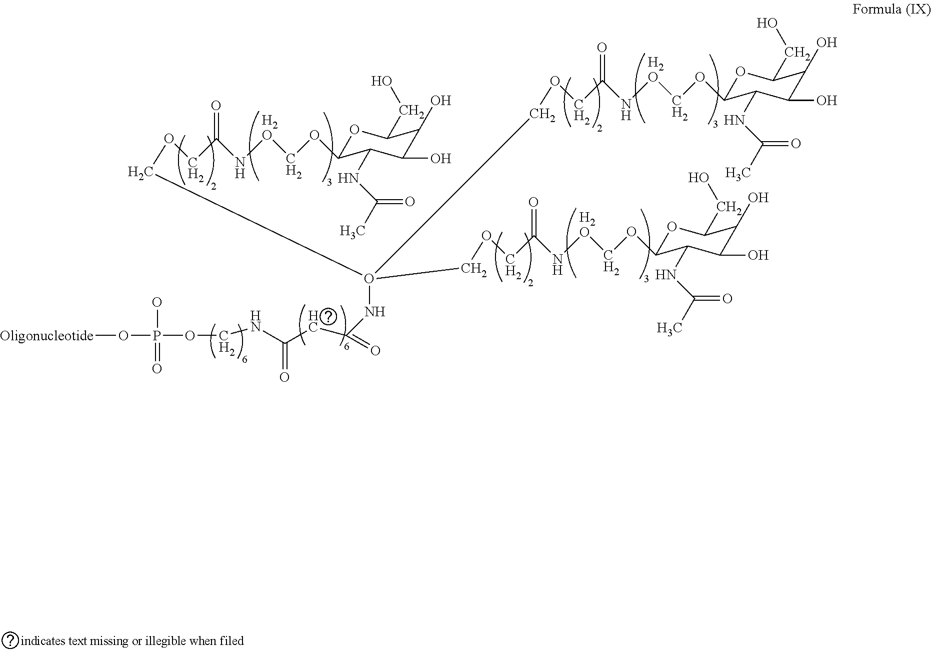

35. A compound according to Clause 33 or 34, wherein the oligonucleoside comprises an RNA duplex which further comprises one or more riboses modified at the 2′ position, preferably a plurality of riboses modified at the 2′ position.
36. A compound according to Clause 35, wherein the modifications are chosen from 2′-O-methyl, 2′-deoxy-fluoro, and 2′-deoxy.
37. A compound according to any of Clauses 33 to 36, wherein the oligonucleoside further comprises one or more degradation protective moieties at one or more ends.
38. A compound according to Clause 37, wherein said one or more degradation protective moieties are not present at the end of the oligonucleoside strand that carries the linker/ligand moieties, and/or wherein said one or more degradation protective moieties is selected from phosphorothioate internucleoside linkages, phosphorodithioate internucleoside linkages and inverted abasic nucleosides, wherein said inverted abasic nucleosides are present at the distal end of the same strand to the end that carries the linker/ligand moieties.
39. A compound according to Clause 33, wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 5′ end of its second strand to the adjacent phosphate.
40. A compound according to Clause 34, wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 3′ end of its second strand to the adjacent phosphate.
41. A process of preparing a compound according to any of Clauses 1 to 40, which comprises reacting compounds of Formulae (X) and (XI):

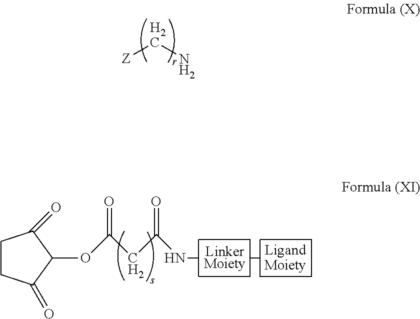

wherein:
r and s are independently an integer selected from 1 to 16; and
Z is an oligonucleoside moiety;
and where appropriate carrying out deprotection of the ligand and/or annealing of a second strand for the oligonucleoside.
42. A process according to Clause 41, to prepare a compound according to any of Clauses 6, 8 to 14, 16 to 33, and 35 to 40, wherein: compound of Formula (X) is Formula (Xa):

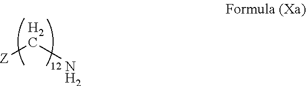

and compound of Formula (XI) is Formula (XIa):

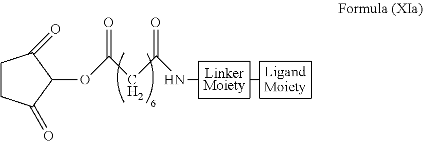

wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 5′ end of its second strand to the adjacent phosphate.
43. A process according to Clause 41, to prepare a compound according to any of Clauses 5, 7, 9 to 13, 15 to 32, and 34 to 40, wherein:
compound of Formula (X) is Formula (Xb):

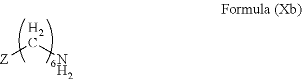

and compound of Formula (XI) is Formula (XIa):

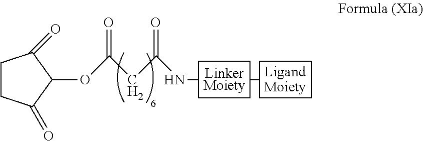

wherein the oligonucleoside comprises an RNA duplex comprising first and second strands, wherein the first strand is at least partially complementary to an RNA sequence of a target gene, and the second strand is at least partially complementary to said first strand, and wherein each of the first and second strands have 5′ and 3′ ends, and wherein said RNA duplex is attached at the 3′ end of its second strand to the adjacent phosphate.
44. A process according to Clauses 42 or 43, wherein:
compound of Formula (XIa) is Formula (XIb):

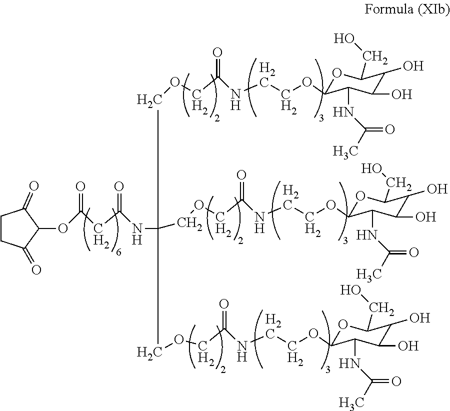

45. A compound of Formula (X):

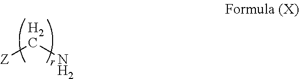

wherein:
r is independently an integer selected from 1 to 16; and
Z is an oligonucleoside moiety.
46. A compound of Formula (Xa):

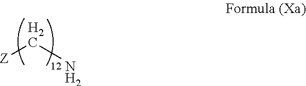

47. A compound of Formula (Xb):

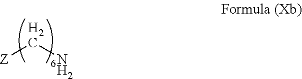

48. A compound of Formula (XI):

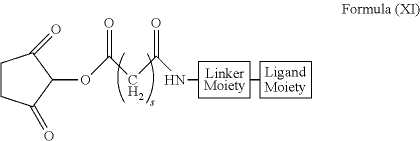

wherein:
s is independently an integer selected from 1 to 16; and
Z is an oligonucleoside moiety.
49. A compound of Formula (XIa):

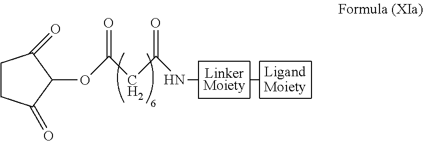

50. A compound of Formula (XIb):

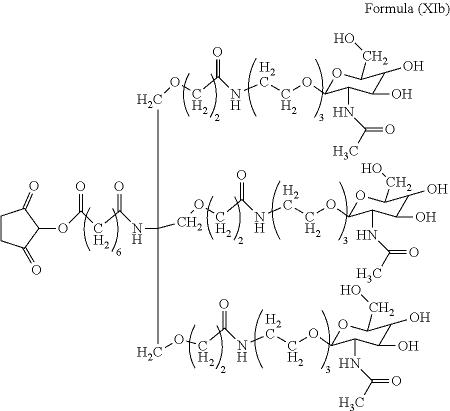

51. Use of a compound according to any of Clauses 45 and 48 to 50, for the preparation of a compound according to any of Clauses 1 to 40.
52. Use of a compound according to Clause 46, for the preparation of a compound according to any of Clauses 6, 8 to 14, 16 to 33, and 35 to 40.
53. Use of a compound according to Clause 47, for the preparation of a compound according to any of Clauses 5, 7, 9 to 13, 15 to 32, and 34 to 40.
54. A compound or composition obtained, or obtainable by a process according to any of Clauses 41 to 44.
55. A pharmaceutical composition comprising of a compound according to any of Clauses 1 to 40, together with a pharmaceutically acceptable carrier, diluent or excipient.
56. A compound according to any of Clauses 1 to 40, for use in therapy.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended Clauses.

The following constructs are used in the examples

TABLE 1

| Target | ID | Sense Sequence 5′ → 3′ | Antisense Sequence 5′ → 3′ |
|---|---|---|---|
| hsHAO1 | ETX005 | (invabasic)(invabasic)gsascuuuCfaUfCfCfuggaaauasusa | usAfsuauUfuCfCfaggaUfgAfaagucscsa |
| | | (NHC6)(MFCO)(ET-GalNAc-TIN3) | |
| hsHAO1 | ETX001 | (ET-GaINAc-TIN3)(MFCO)(NH-DEG) | usAfsuauUfuCfCfaggaUfgAfaagucscsa |
| | | gacuuuCfaUfCfCfuggaaauasusa(invabasic)(invabasic) | |
| hsC5 | ETX014 | (invabasic)(invabasic)asasGfcAfaGfaUfA | usAfsUfuAfuaAfaAfauaUfcUfuGfcuusu |
| | | fUfuUfuuAfuAfaua(NHC6)(MFCO)(ET-GalNAc-TIN3) | sudTdT |
| hsC5 | ETX010 | (ET-GalNAc-TIN3)(MFCO)(NH-DEG)aaGfcAfaGfaUfAfUfu | usAfsUfuAfuaAfaAfauaUfcUfuGfcuusu |
| | | UfuuAfuAfasusa(invabasic)(invabasic) | sudTdT |
| hsTTR | ETX019 | (ET-GaINAc-TIN3)(MFCO)(NH-DEG) | usCfsuugGfuuAfcaugAfaAfucccasusc |
| | | ugggauUfuCfAfUfguaaccaasgsa(invabasic)(invabasic) | |
| hsTTR | ETX023 | (invabasic)(invabasic)usgsggauUfuCfAfUfguaaccaaga | usCfsuugGfuu AfcaugAfaAfucccasusc |
| | | (NHC6)(MFCO)(ET-GalNAc-TIN3) | |

In Table 1 the components in brackets having the following nomenclature (ET-GalNAc-T1N3), (MFCO), and (NH-DEG) are descriptors of elements of the linkers, and the complete corresponding linker structures are shown in FIG. 30 and FIG. 31 herein. This correspondence of abbreviation to actual linker structure similarly applies to all other references of the above abbreviations herein.

Reference to (invabasic)(invabasic) refers to nucleosides in an overall polynucleoside which are the terminal 2 nucleosides which have sugar moieties that are (i) abasic, and (ii) in an inverted configuration, whereby the bond between the penultimate nucleoside and the antepenultimate nucleoside has a reversed linkage, namely either a 5-5 or a 3-3 linkage. Again, this similarly applies to all other references to (invabasic)(invabasic) herein.

TABLE 1A

| | | | Linker plus ligand |
|---|---|---|---|
| Target | ID | Short Descriptor | SiRNA as Table 1 |
| hsHAO1 | ETX005 | 3′-GalNAc T1a | Linker + ligand as FIG. 30 |
| | | inverted abasic | |
| hsHAO1 | ETX001 | 5′-GalNAc T1b | Linker + ligand as FIG. 31 |
| | | inverted abasic | |
| hsC5 | ETX014 | 3′-GalNAc T1a | Linker + ligand as FIG. 30 |
| | | inverted abasic | |
| hsC5 | ETX010 | 5′-GalNAc T1b | Linker + ligand as FIG. 31 |
| | | inverted abasic | |
| hsTTR | ETX019 | 5′-GalNAc T1b | Linker + ligand as FIG. 31 |
| | | inverted abasic | |
| hsTTR | ETX023 | 3′-GalNAc T1a | Linker + ligand as FIG. 30 |
| | | inverted abasic | |

It should also be understood as already explained herein with reference to FIG. 30/FIG. 31, that where appropriate for the linker portions as shown in FIG. 30/FIG. 31 which can be present in any of products ETX001, ETX005, ETX010, ETX014, ETX019, ETX023 according to the present invention, that while these products can include molecules based on the linker and ligand portions as specifically depicted in FIG. 30/FIG. 31 attached to an oligonucleoside moiety as also depicted herein, these products may alternatively further comprise, or consist essentially of, molecules wherein the linker and ligand portions are essentially as depicted in FIG. 30/FIG. 31 attached to an oligonucleoside moiety but having the F substituent as shown in FIG. 30/FIG. 31 on the cyclo-octyl ring replaced by a substituent occurring as a result of hydrolytic displacement, such as an OH substituent. In this way, (a) these products can consist essentially of molecules having linker and ligand portions specifically as depicted in FIG. 30/FIG. 31, with a F substituent on the cyclo-octyl ring; or (b) these products can consist essentially of molecules having linker and ligand portions essentially as depicted in FIG. 30/FIG. 31 but having the F substituent as shown in FIG. 30/FIG. 31 on the cyclo-octyl ring replaced by a substituent occurring as a result of hydrolytic displacement, such as an OH substituent, or (c) these products can comprise a mixture of molecules as defined in (a) or (b).

The following control constructs are also used in the examples:

TABLE 2

| Target | ID | Sense Sequence 5′ → 3′ | Antisense Sequence 5′ → 3′ |
|---|---|---|---|
| F-Luc | XD- | cuuAcGcuGAGuAcuucGAdTsdT | UCGAAGuACUCAGCGuAAGdTsdT |
| | 00914 | | |
| hsFVII | XD- | AGAuAuGcAcAcAcAcGGAdTsdT | UCCGUGUGUGUGcAuAUCUdTsdT |
| | 03999 | | |
| hsAHSA1 | XD- | uscsUfcGfuGfgCfcUfuAfaU | UfsUfsuCfaUfuAfaGfgCfcA |
| | 15421 | fgAfaAf(invdT) | fcGfaGfasusu |

### Abbreviations

AHSA1 Activator of heat shock protein ATPase1
ASGR1 Asialoglycoprotein Receptor 1
ASO Antisense oligonucleoside
bDNA branched DNA
bp base-pair
C5 complement C5
conc. concentration
ctrl. control
CV coefficient of variation
dG, dC, dA, dT DNA residues
F Fluoro
FCS fetal calf serum
GalNAc N-Acetylgalactosamine
GAPDH Glyceraldehyde 3-phosphate dehydrogenase
G, C, A, U RNA residues
g, c, a, u 2′-O-Methyl modified residues
Gf, Cf, Af, Uf 2′-Fluoro modified residues
h hour
HAO1 Hydroxyacid Oxidase 1
HPLC High performance liquid chromatography
Hs Homo sapiens
IC50 concentration of an inhibitor where the response is reduced by 50%
ID identifier
KD knockdown
LF2000 Lipofectamine2000
M molar
Mf Macaca fascicularis
min minute
MV mean value
n.a. or N/A not applicable
NEAA non-essential amino acid
nt nucleoside
QC Quality control
QG2.0 QuantiGene 2.0
RLU relative light unit
RNAi RNA interference
RT room temperature
s Phosphorothioate backbone modification
SAR structure-activity relationship
SD standard deviation
siRNA small interfering RNA
TTR Transthyretin

### Example 1

### Summary

GalNAc-siRNAs targeting either hsHAO1, hsC5 or hsTTR mRNA were synthesized and QC-ed. The entire set of siRNAs (except siRNAs targeting HAO1) was first studied in a dose-response setup in HepG2 cells by transfection using RNAiMAX, followed by a dose-response analysis in a gymnotic free uptake setup in primary human hepatocytes.

Direct incubation of primary human hepatocytes with GalNAc-siRNAs targeting hsHAO1, hsC5 or hsTTR mRNA resulted in dose-dependent on-target mRNA silencing to varying degrees.

### Aim of Study

The aim of this set of experiments was to analyze the in vitro activity of different GalNAc-ligands in the context of siRNAs targeting three different on-targets, namely hsHAO1, hsC5 or hsTTR mRNA.

Work packages of this study included (i) assay development to design, synthesize and test bDNA probe sets specific for each and every individual on-target of interest, (ii) to identify a cell line suitable for subsequent screening experiments, (iii) dose-response analysis of potentially all siRNAs (by transfection) in one or more human cancer cell lines, and (iv) dose-response analysis of siRNAs in primary human hepatocytes in a gymnotic, free uptake setting. In both settings, IC50 values and maximal inhibition values should be calculated followed by ranking of the siRNA study set according to their potency.

### Material and Methods

### Oligonucleoside Synthesis

Standard solid-phase synthesis methods were used to chemically synthesize siRNAs of interest (see Table 1) as well as controls (see Table 2).

### Cell Culture and In-Vitro Transfection Experiments

Cell culture, transfection and QuantiGene2.0 branched DNA assay are described below, and siRNA sequences are listed in Tables 1 and 2. HepG2 cells were supplied by American Tissue Culture Collection (ATCC) (HB-8065, Lot #: 63176294) and cultured in ATCC-formulated Eagle's Minimum Essential Medium supplemented to contain 10% fetal calf serum (FCS). Primary human hepatocytes (PHHs) were sourced from Primacyt (Schwerin, Germany) (Lot #: CyHuf19009HEc). Cells are derived from a malignant glioblastoma tumor by explant technique. All cells used in this study were cultured at 37° C. in an atmosphere with 5% CO₂ in a humidified incubator.

For transfection of HepG2 cells with hsC5 or hsTTR targeting siRNAs (and controls), cells were seeded at a density of 20.000 cells/well in regular 96-well tissue culture plates. Transfection of cells with siRNAs was carried out using the commercially available transfection reagent RNAiMAX (Invitrogen/Life Technologies) according to the manufacturer's instructions. 10 point dose-response experiments of 20 candidates (11×hsC5, 9×hsTTR) were done in HepG2 cells with final siRNA concentrations of 24, 6, 1.5, 0.4, 0.1, 0.03, 0.008, 0.002, 0.0005 and 0.0001 nM, respectively.

Dose response analysis in PHHs was done by direct incubation of cells in a gymnotic, free uptake setting starting with 1.5 μM highest final siRNA concentration, followed by 500 nM and from there on going serially down in twofold dilution steps.

Control wells were transfected into HepG2 cells or directly incubated with primary human hepatocytes at the highest test siRNA concentrations studied on the corresponding plate. All control siRNAs included in the different project phases next to mock treatment of cells are summarized and listed in Table 2. For each siRNA and control, at least four wells were transfected/directly incubated in parallel, and individual data points were collected from each well.

After 24 h of incubation with siRNA post-transfection, media was removed and HepG2 cells were lysed in Lysis Mixture (1 volume of lysis buffer plus 2 volumes of nuclease-free water) and then incubated at 53° C. for at least 45 minutes. In the case of PHHs, plating media was removed 5 h post treatment of cells followed by addition of 50 μl of complete maintenance medium per well. Media was exchanged in that way every 24 h up to a total incubation period of 72 h. At either 4 h or 72 h time point, cell culture supernatant was removed followed by addition of 200 μl of Lysis Mixture supplemented with 1:1000 v/v of Proteinase K.

The branched DNA (bDNA) assay was performed according to manufacturer's instructions. Luminescence was read using a 1420 Luminescence Counter (WALLAC VICTOR Light, Perkin Elmer, Rodgau-Jugesheim, Germany) following 30 minutes incubation in the presence of substrate in the dark. For each well, the on-target mRNA levels were normalized to the hsGAPDH mRNA levels. The activity of any siRNA was expressed as percent on-target mRNA concentration (normalized to hsGAPDH mRNA) in treated cells, relative to the mean on-target mRNA concentration (normalized to hsGAPDH mRNA) across control wells.

### Assay Development

QuantiGene2.0 branched DNA (bDNA) probe sets were designed and synthesised specific for Homo sapiens GAPDH, AHSA1, hsHAO1, hsC5 and hsTTR. bDNA probe sets were initially tested by bDNA analysis according to manufacturer's instructions, with evaluation of levels of mRNAs of interest in two different lysate amounts, namely 10 μl and 50 μl, of the following human and monkey cancer cell lines next to primary human hepatocytes: SJSA-1, TF1, NCI-H1650, Y-79, Kasumi-1, EAhy926, Caki-1, Colo205, RPTEC, A253, HeLaS3, Hep3B, BxPC3, DU145, THP-1, NCI-H460, IGR37, LS174T, Be(2)-C, SW 1573, NCI-H358, TC71, 22Rv1, BT474, HeLa, KBwt, Panc-1, U87MG, A172, C42, HepG2, LNCaP, PC3, SupT1l, A549, HCT116, HuH7, MCF7, SH-SY5Y, HUVEC, C33A, HEK293, HT29, MOLM 13 and SK-MEL-2. Wells containing only bDNA probe set without the addition of cell lysate were used to monitor technical background and noise signal.

### Results

### Identification of Suitable Cell Types for Screening of GalNAc-siRNas

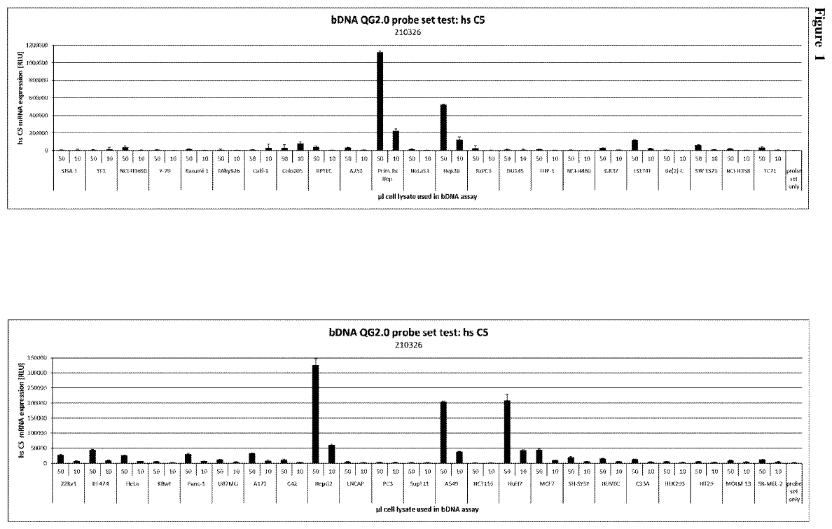

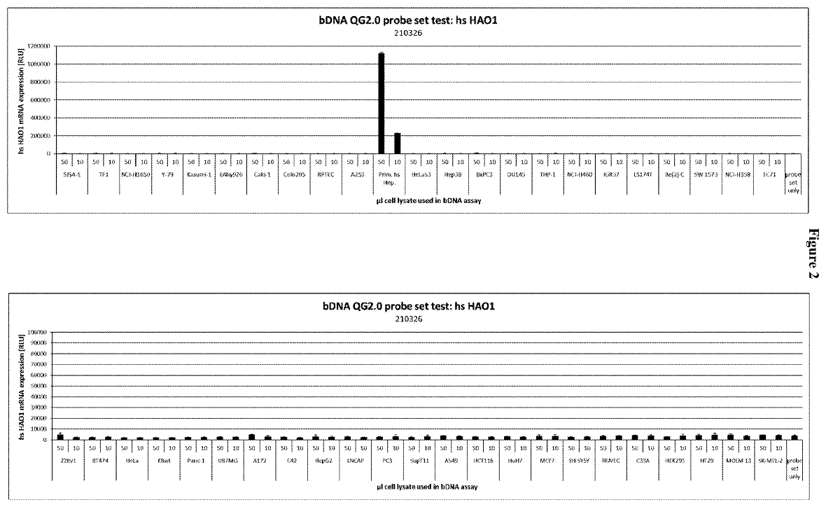

FIG. 1 to FIG. 3 show mRNA expression data for the three on-targets of interest, namely hsC5, hsHAO1 and hsTTR, in lysates of a diverse set of human cancer cell lines plus primary human hepatocytes. Cell numbers per lysate volume are identical with each cell line tested, this is necessary to allow comparisons of expression levels amongst different cell types. FIG. 1 shows hsC5 mRNA expression data for all cell types tested.

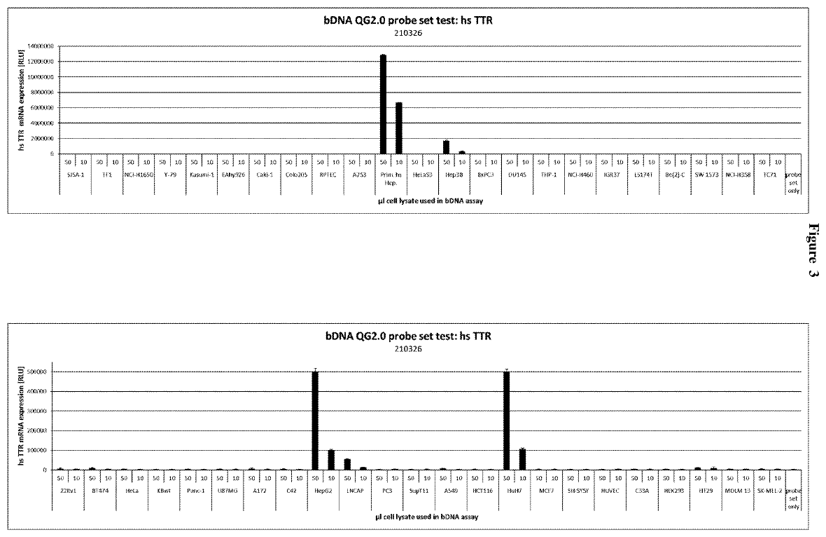

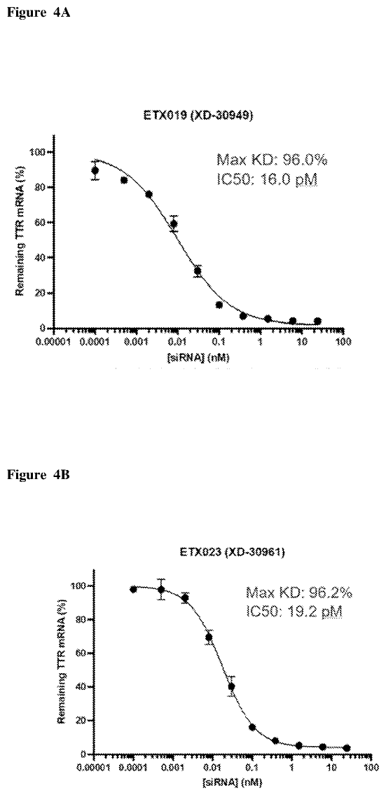

The identical type of cells were also screened for expression of hsHAO1 mRNA, results are shown in bar diagrams as part of FIG. 2.

Lastly, suitable cell types were identified which would allow for screening of GalNAc-siRNAs targeting hsTTR, respective data are part of FIG. 3.

In summary, mRNA expression levels for all three on-targets of interest are high enough in primary human hepatocytes (PHHs). Further, HepG2 cells could be used to screen GalNAc-siRNAs targeting hsC5 and hsTTR mRNAs, in contrast, no cancer cell line could be identified which would be suitable to test siRNAs specific for hsHAO1 mRNA.

Dose-Response Analysis of hsTTR Targeting GalNAc-siRNAs in HepG2 Cells

Following transfection optimization, HepG2 cells were transfected with the entire set of hsTTR targeting GalNAc-siRNAs (see Table 1) in a dose-response setup using RNAiMAX. The highest final siRNA test concentration was 24 nM, going down in nine fourfold dilution steps. The experiment ended at 4 h and 24 h post transfection of HepG2 cells. Table 3 lists activity data for all hsTTR targeting GalNAC-siRNAs studied.

TABLE 3
Target, incubation time, external ID, IC20/IC50/IC80 values and
maximal inhibition of hsTTR targeting siRNAs in HepG2 cells.
The listing is ordered according to external ID, with
4 h of incubation listed on top and 24 h of incubation on the bottom.

| | Incu- | | | | | Max. |
|---|---|---|---|---|---|---|
| | bation | External | IC20 | IC50 | IC80 | Inhib. |
| Target | [h] | ID | [nM] | [nM] | [nM] | [%] |
| hsTTR | 4 | ETX019 | 0.206 | 3.769 | #N/A | 58.4 |
| hsTTR | 4 | ETX023 | 1.338 | #N/A | #N/A | 45.9 |
| hsTTR | 24 | ETX019 | 0.002 | 0.016 | 0.143 | 96.0 |
| hsTTR | 24 | ETX023 | 0.005 | 0.019 | 0.081 | 96.2 |

Results for the 24 h incubation are also shown in FIGS. 4A-B

In general, transfection of HepG2 cells with hsTTR targeting siRNAs results in on-target mRNA silencing spanning in general the entire activity range from 0% silencing to maximal inhibition. Data generated 24 h post transfection are more robust with lower standard variations, as compared to data generated only 4 h post transfection. Further, the extent of on-target knockdown generally increases over time from 4 h up to 24 h of incubation. hsTTR GalNAc-siRNAs have been identified that silence the on-target mRNA>95% with IC50 values in the low double-digit pM range.

Dose-Response Analysis of hsC5 Targeting GalNAc-siRNAs in HepG2 Cells

The second target of interest, hsC5 mRNA, was tested in an identical dose-response setup (with minimally different final siRNA test concentrations, however) by transfection of HepG2 cells using RNAiMAX with GalNAc-siRNAs sharing identical linger/position/GalNAc-ligand variations as with hsTTR siRNAs, but sequences specific for the on-target hsC5 mRNA.

TABLE 4
Target, incubation time, external ID, IC20/IC50/IC80 values and
maximal inhibition of hsC5 targeting siRNAs in HepG2 cells.
The listing is ordered according to external ID, with 4 h
of incubation listed on top and 24 h of incubation on the bottom.

| | Incu- | | | | | Max. |
|---|---|---|---|---|---|---|
| | bation | External | IC20 | IC50 | IC80 | Inhib. |
| Target | [h] | ID | [nM] | [nM] | [nM] | [%] |
| C5 | 4 | ETX010 | 0.125 | 0.445 | #N/A | 71.1 |
| C5 | 4 | ETX014 | 0.595 | 2.554 | #N/A | 52.7 |
| C5 | 24 | ETX010 | 0.003 | 0.011 | 0.064 | 88.3 |
| C5 | 24 | ETX014 | 0.003 | 0.014 | 0.130 | 88.3 |

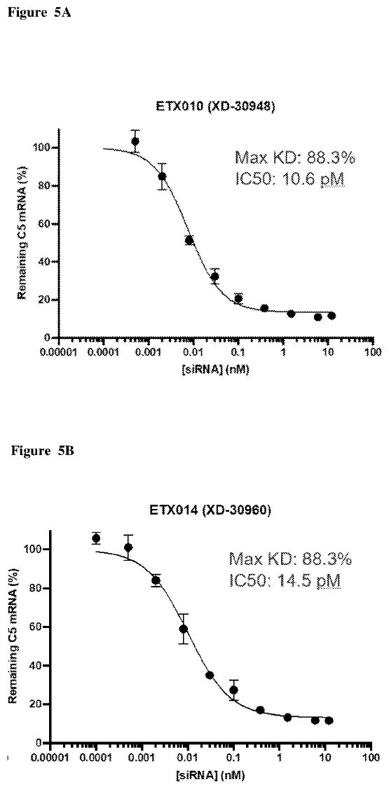

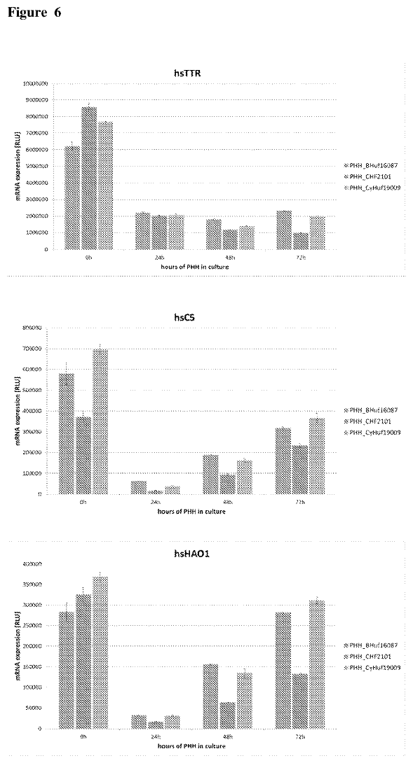

Results for the 24 h incubation are also shown in FIGS. 5A-B

There is dose-dependent on-target hsC5 mRNA silencing upon transfection of HepG2 cells with the GalNAc-siRNA set specific for hsC5. Some knockdown can already be detected at 4 h post-transfection of cells, an even higher on-target silencing is observed after a longer incubation period, namely 24 h. hsC5 GalNAc-siRNAs have been identified that silence the on-target mRNA almost 90% with IC50 values in the low single-digit pM range.

### Identification of a Primary Human Hepatocyte Batch Suitable for Testing of all GalNAc-siRNAs

The dose-response analysis of the two GalNAc-siRNA sets in human cancer cell line HepG2 should demonstrate (and ensure) that all new GalNAc-/linker/position/cap variants are indeed substrates for efficient binding to AGO2 and loading into RISC, and in addition, able to function in RNAi-mediated cleavage of target mRNA. However, in order to test whether the targeting GalNAc-ligand derivatives allow for efficient uptake into hepatocytes, dose-response analysis experiments should be done in primary human hepatocytes by gymnotic, free uptake setup. Hepatocytes do exclusively express the Asialoglycoprotein receptor (ASGR1) to high levels, and this receptor generally is used by the liver to remove target glycoproteins from circulation. It is common knowledge by now, that certain types of oligonucleosides, e.g. siRNAs or ASOs, conjugated to GalNAc-ligands are recognized by this high turnover receptor and efficiently taken up into the cytoplasm via clathrin-coated vesicles and trafficking to endosomal compartments. Endosomal escape is thought to be the rate-limiting step for oligonucleoside delivery.

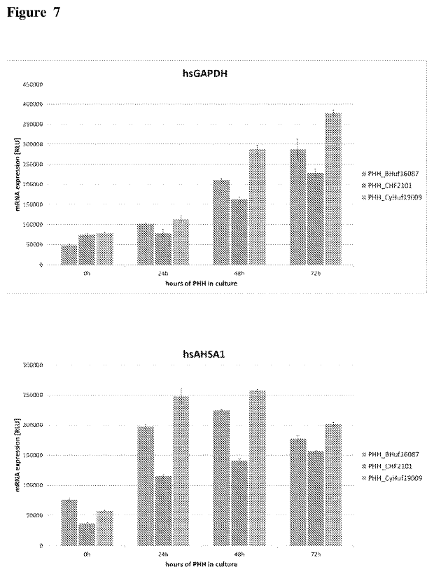

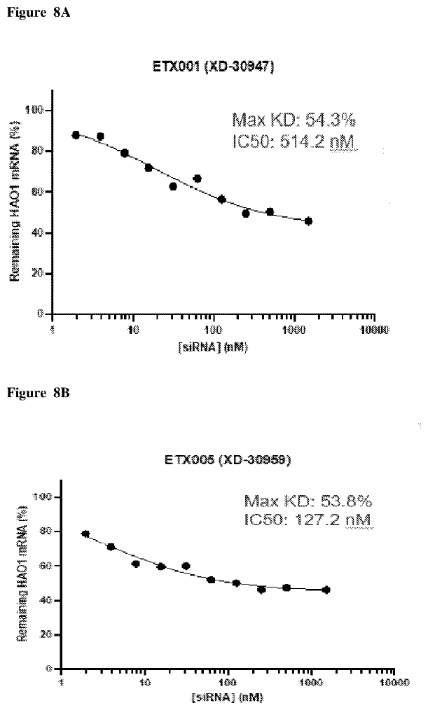

An intermediate assay development experiment was done in which different batches of primary human hepatocytes were tested for their expression levels of relevant genes of interest, namely hsC5, hsTTR, hsHAO1, hsGAPDH and hsAHSA1. Primacyt (Schwerin, Germany) provided three vials of different primary human hepatocyte batches for testing, namely BHuf16087, CHF2101 and CyHuf19009. The cells were seeded on collagen-coated 96-well tissue culture plates, followed by incubation of cells for 0 h, 24 h, 48 h and 72 h before cell lysis and bDNA analysis to monitor mRNA levels of interest. FIG. 6 shows the absolute mRNA expression data for all three on-targets of interest—hsTTR, hsC5 and hsHAO1—in the primary human hepatocyte batches BHuf16087, CHF2101 and CyHuf19009. mRNA expression levels of hsGAPDH and hsAHSA1 are shown in FIG. 7.

In FIGS. 6 and 7 the left hand column of each data set triplet is BHuf16087, the middle column is CHF2101 and the right hand column is CyHuf19009.

Overall, the mRNA expression of all three on-targets of interest in the primary human hepatocyte batches BHuf16087 and CyHuf19009 are high enough after 72 h to continue with the bDNA assay. Due to the total amount of vials available for further experiments, we continued the experiments with the batch CyHuf19009.

Dose-Response Analysis of hsHAO1 Targeting GalNAc-siRNAs in PHHs

Following the identification of a suitable batch (CyHuf19009) of primary human hepatocytes (PHHs), a gymnotic, free uptake analysis was performed of hsHAO1 targeting GalNAc-siRNAs, listed in Table 1. The highest tested final siRNA concentration was 1.5 μM, followed by 500 nM, going down in eight two-fold serial dilution steps to the lowest final siRNA concentration of 1.95 nM. The experiments ended at 4 h and 72 h post direct incubation of PHH cells. Table 5 lists activity data for all hsHAO1 targeting GalNAc-siRNAs studied. All control siRNAs included in this experiment are summarized and listed in Table 2.

TABLE 5
Target, incubation time, external ID, IC20/IC50/IC80 values and
maximal inhibition of hsHAO1 targeting GalNAc-siRNAs in
primary human hepatocytes (PHHs). The listing is
organized according to external ID, with 4 h and 72 h
incubation listed on top and bottom, respectively.

| | Incu- | | | | | Max. |
|---|---|---|---|---|---|---|
| | bation | External | IC20 | IC50 | IC80 | Inhib. |
| Target | [h] | ID | [nM] | [nM] | [nM] | [%] |
| hsHAO1 | 4 | ETX001 | #N/A | #N/A | #N/A | 3.5 |
| (hsGO1) | | | | | | |
| hsHAO1 | 4 | ETX005 | #N/A | #N/A | #N/A | 0.7 |
| (hsGO1) | | | | | | |
| hsHAO1 | 72 | ETX001 | 7.1 | 514.2 | #N/A | 54.3 |
| (hsGO1) | | | | | | |
| hsHAO1 | 72 | ETX005 | 1.5 | 127.2 | #N/A | 53.8 |
| (hsGO1) | | | | | | |

Results for the 72 h incubation are also shown in FIGS. 8A-B.

Gymnotic, free uptake of GalNAc-siRNAs targeting hsHAO1 did not lead to significant on-target silencing within 4 h, however after 72 h incubation on-target silencing was visible in a range of 35.5 to 58.1% maximal inhibition.

Dose-Response Analysis of hsC5 Targeting GalNAc-siRNAs in PHHs

The second target of interest, hsC5 mRNA, was tested in an identical dose-response setup by gymnotic, free uptake in PHHs with GalNAc-siRNAs sharing identical linker/position/GalNAc-ligand variations as with hsTTR and hsHAO1 tested in the assays before, but sequences specific for the on-target hsC5 mRNA. Sequences for the GalNAc-siRNAs targeting hsC5 and all sequences and information about control siRNAs are listed in Table 1 and Table 2, respectively. The experiment ended after 4 h and 72 h direct incubation of PHHs. Table 6 lists activity data for all hsC5 targeting GalNAc-siRNAs studied.

TABLE 6
Target, incubation time, external ID, IC20/IC50/IC80 values and
maximal inhibition of hsC5 targeting GalNAc-siRNAs in PHHs.
The listing is organized according to external ID,
with 4 h and 72 h incubation listed on top and bottom, respectively.

| | Incu- | | | | | Max. |
|---|---|---|---|---|---|---|
| | bation | External | IC20 | IC50 | IC80 | Inhib. |
| Target | [h] | ID | [nM] | [nM] | [nM] | [%] |
| C5 | 4 | ETX010 | #N/A | #N/A | #N/A | −1.3 |
| C5 | 4 | ETX014 | 51.8 | #N/A | #N/A | 23.7 |
| C5 | 72 | ETX010 | 4.3 | 72.1 | #N/A | 64.9 |
| C5 | 72 | ETX014 | 2.2 | 63.7 | #N/A | 65.6 |

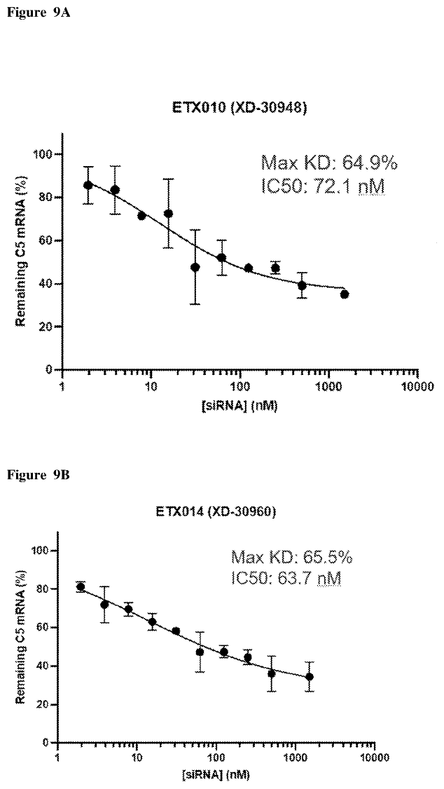

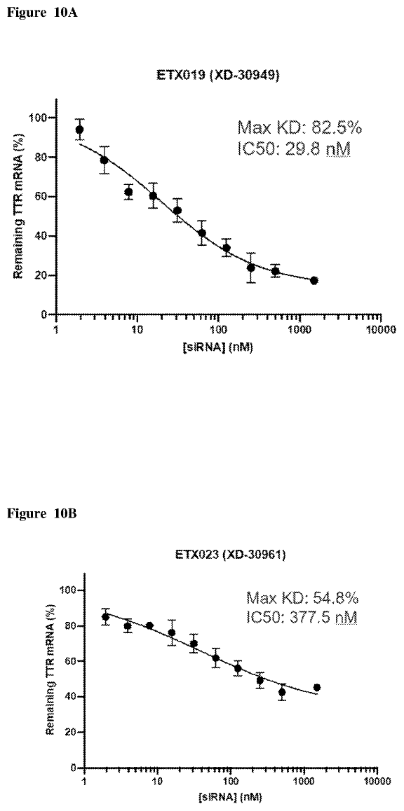

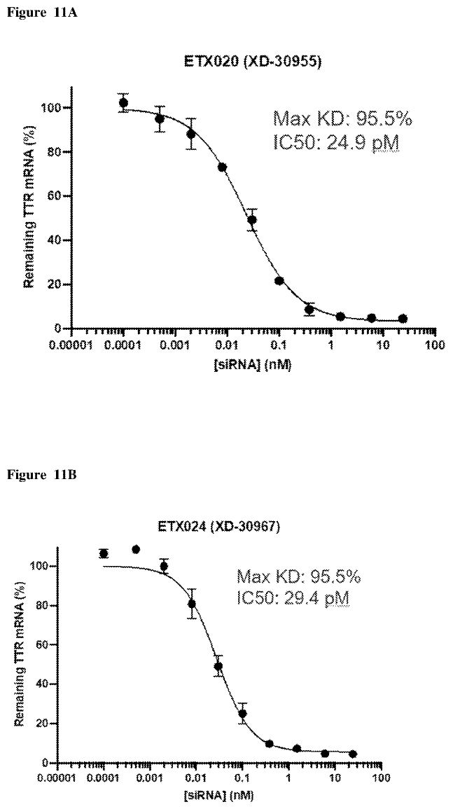

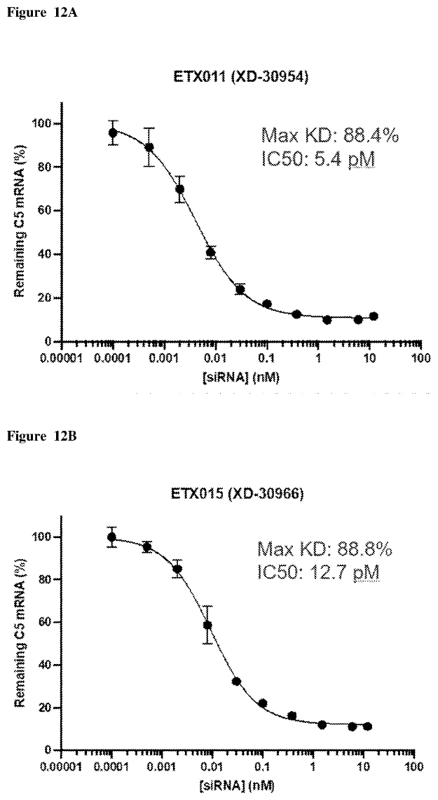

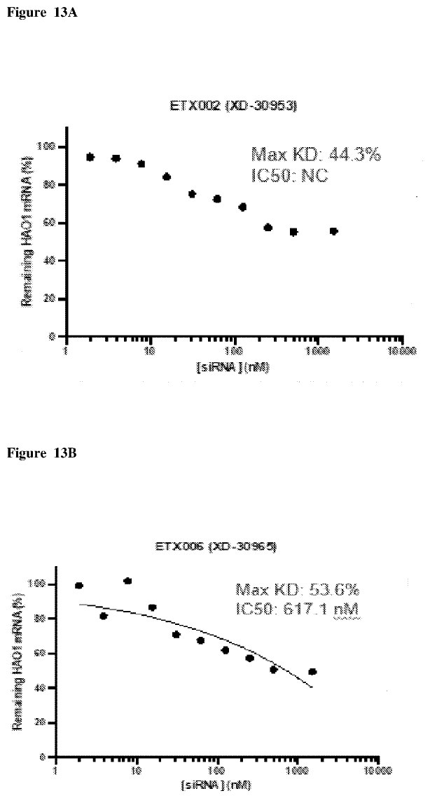

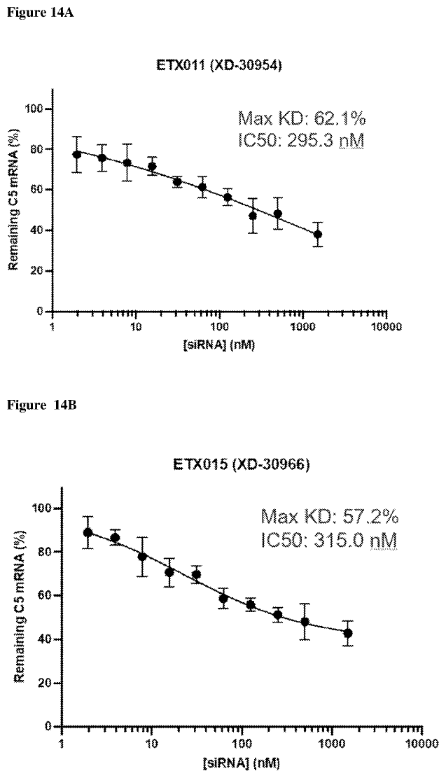

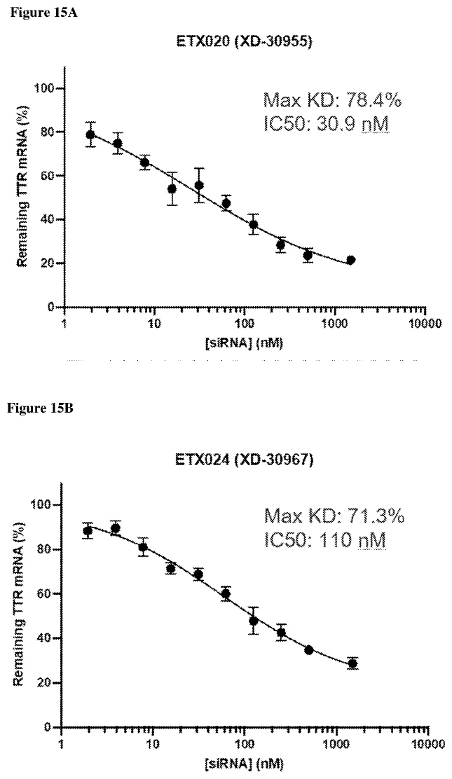

Results for the 72 h incubation are also shown in FIGS. 9A-B.

No significant on-target silencing of GalNAc-siRNAs is visible after 4 h incubation. Data generated after an incubation period of 72 h showed a more robust on-target silencing of up to 65.5% maximal inhibition.

Dose-Response Analysis of hsTTR Targeting GalNAc-siRNAs in PHHs

The last target of interest, hsTTR mRNA, was again tested in a gymnotic, free uptake in PHHs in an identical dose-response setup as for the targets hsHAO1 and hsC5, with the only difference being that specific siRNA sequences for the on-target hsTTR mRNA was used (see Table 1).

The experiment ended after 72 h of direct incubation of PHHs. Table 7 lists activity data for all hsTTR targeting GalNAc-siRNAs studied.

TABLE 7
Target, incubation time, external ID, IC20/IC50/IC80 values
and maximal inhibition of hsTTR targeting GalNAc-siRNAs in
primary human hepatocytes (PHHs). The listing is
organized according to external ID.

| | Incu- | | | | | Max. |
|---|---|---|---|---|---|---|
| | bation | External | IC20 | IC50 | IC80 | Inhib. |
| Target | [h] | ID | [nM] | [nM] | [nM] | [%] |
| hsTTR | 72 | ETX019 | 3.9 | 29.8 | 1536.8 | 82.5 |
| hsTTR | 72 | ETX023 | 6.7 | 377.5 | #N/A | 54.8 |

Results are also shown in FIGS. 10A-B.

Gymnotic, free uptake of GalNAc-siRNAs targeting hsTTR did lead to significant on-target silencing within 72 h, ranging between 46 to 82.5% maximal inhibition. hsTTR GalNAc-siRNAs were identified that silence the on-target mRNA with IC50 values in the low double-digit nM range.

### Conclusions and Discussion

The scope of this study was to analyze the in vitro activity of GalNAc-ligands according to the present invention when used in the context of siRNAs targeting three different on-targets, namely hsHAO1, hsC5 and hsTTR mRNA. siRNA sets specific for each target were composed of siRNAs with different linker/cap/modification/GalNAc-ligand chemistries in the context of two different antisense strands each.

For all targets, GalNAc-siRNAs from Table 1 were identified that showed a high overall potency and low IC50 value.

### 1.1 Example 2

### Routes of Synthesis

### i) Synthesis of the Conjugate Building Blocks TriGalNAc

Thin layer chromatography (TLC) was performed on silica-coated aluminium plates with fluorescence indicator 254 nm from Macherey-Nagel. Compounds were visualized under UV light (254 nm), or after spraying with the 5% H₂SO₄ in methanol (MeOH) or ninhydrin reagent according to Stahl (from Sigma-Aldrich), followed by heating. Flash chromatography was performed with a Biotage Isolera One flash chromatography instrument equipped with a dual variable UV wavelength detector (200-400 nm) using Biotage Sfär Silica 10, 25, 50 or 100 g columns (Uppsala, Sweden).

All moisture-sensitive reactions were carried out under anhydrous conditions using dry glassware, anhydrous solvents and argon atmosphere. All commercially available reagents were purchased from Sigma-Aldrich and solvents from Carl Roth GmbH+Co. KG. D-Galactosamine pentaacetate was purchased from AK scientific.

HPLC/ESI-MS was performed on a Dionex UltiMate 3000 RS UHPLC system and Thermo Scientific MSQ Plus Mass spectrometer using an Acquity UPLC Protein BEH C4 column from Waters (300 Å, 1.7 μm, 2.1×100 mm) at 60° C. The solvent system consisted of solvent A with H₂O containing 0.1% formic acid and solvent B with acetonitrile (ACN) containing 0.1% formic acid. A gradient from 5-100% of B over 15 min with a flow rate of 0.4 mL/min was employed. Detector and conditions: Corona ultra-charged aerosol detection (from esa). Nebulizer Temp.: 25° C. N₂ pressure: 35.1 psi. Filter: Corona.

¹H and ¹³C NMR spectra were recorded at room temperature on a Varian spectrometer at 500 MHz (1H NMR) and 125 MHz (¹³C NMR). Chemical shifts are given in ppm referenced to the solvent residual peak (CDCl₃—¹H NMR: δ at 7.26 ppm and ¹³C NMR δ at 77.2 ppm; DMSO-d₆—1H NMR: δ at 2.50 ppm and ¹³C NMR δ at 39.5 ppm). Coupling constants are given in Hertz. Signal splitting patterns are described as singlet (s), doublet (d), triplet (t) or multiplet (m).

### ii) Synthesis Route for the Conjugate Building Block TriGalNAc

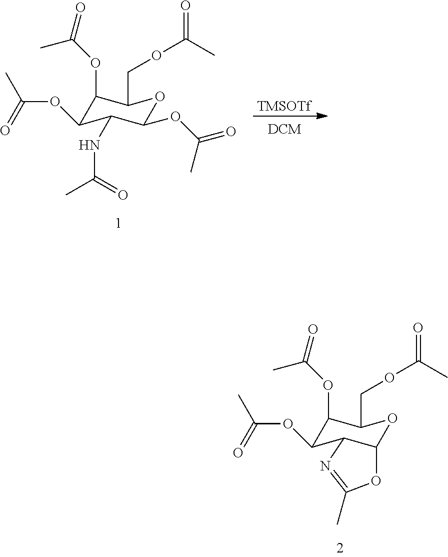 [SMILES: "TMSOTf" ; CC(=O)NC1C(OC(C)=O)OC(COC(C)=O)C(OC(C)=O)C1OC(C)=O ; CC(=O)OCC1OC2OC(C)=NC2C(OC(C)=O)C1OC(C)=O]

Preparation of compound 2: D-Galactosamine pentaacetate (3.00 g, 7.71 mmol, 1.0 eq.) was dissolved in anhydrous dichloromethane (DCM) (30 mL) under argon and trimethylsilyl trifluoromethanesulfonate (TMSOTf, 4.28 g, 19.27 mmol, 2.5 eq.) was added. The reaction was stirred at room temperature for 3 h. The reaction mixture was diluted with DCM (50 mL) and washed with cold saturated aq. NaHCO₃ (100 ml) and water (100 mL). The organic layer was separated, dried over Na2SO4 and concentrated to afford the title compound as yellow oil, which was purified by flash chromatography (gradient elution: 0-10% MeOH in DCM in 10 CV). The product was obtained as colourless oil (2.5 g, 98%, rf=0.45 (2% MeOH in DCM)).

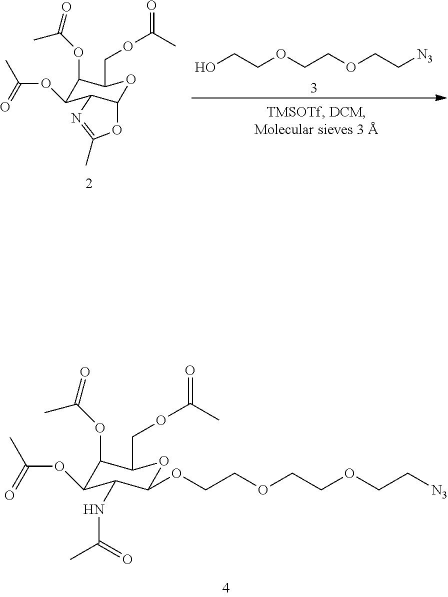

Preparation of compound 4: Compound 2 (2.30 g, 6.98 mmol, 1.0 eq.) and azido-PEG3-OH (1.83 g, 10.5 mmol, 1.5 eq.) were dissolved in anhydrous DCM (40 mL) under argon and molecular sieves 3 Å (5 g) was added to the solution. The mixture was stirred at room temperature for 1 h. TMSOTf (0.77 g, 3.49 mmol, 0.5 eq.) was then added to the mixture and the reaction was stirred overnight. The molecular sieves were filtered, the filtrate was diluted with DCM (100 ml) and washed with cold saturated aq. NaHCO₃ (100 mL) and water (100 mL). The organic layer was separated, dried over Na₂SO₄ and the solvent was removed under reduced pressure. The crude material was purified by flash chromatography (gradient elution: 0-3% MeOH in DCM in 10 CV) to afford the title product as light yellow oil (3.10 g, 88%, rf=0.25 (2% MeOH in DCM)). MS: calculated for C₂₀H₃₂N₄O₁₁, 504.21. Found 505.4. ¹H NMR (500 MHz, CDCl₃) δ 6.21-6.14 (m, 1H), 5.30 (dd, J=3.4, 1.1 Hz, 1H), 5.04 (dd, J=11.2, 3.4 Hz, 1H), 4.76 (d, J=8.6 Hz, 1H), 4.23-4.08 (m, 3H), 3.91-3.80 (m, 3H), 3.74-3.59 (m, 9H), 3.49-3.41 (m, 2H), 2.14 (s, 3H), 2.02 (s, 3H), 1.97 (d, J=4.2 Hz, 6H). ¹³C NMR (125 MHz, CDCl₃) δ 170.6 (C), 170.5 (C), 170.4 (C), 170.3 (C), 102.1 (CH), 71.6 (CH), 70.8 (CH), 70.6 (CH), 70.5 (CH), 70.3 (CH₂), 69.7 (CH₂), 68.5 (CH₂), 66.6 (CH₂), 61.5 (CH₂), 23.1 (CH₃), 20.7 (3×CH₃).

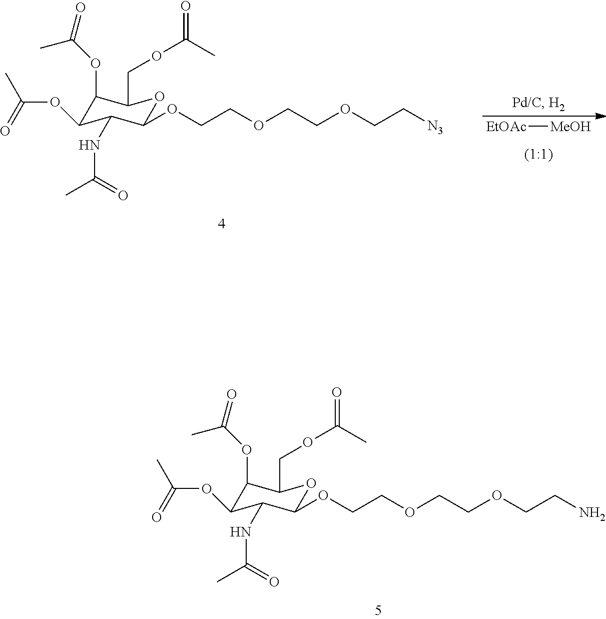

Preparation of compound 5: Compound 4 (1.00 g, 1.98 mmol, 1.0 eq.) was dissolved in a mixture of ethyl acetate (EtOAc) and MeOH (30 mL 1:1 v/v) and Pd/C (100 mg) was added. The reaction mixture was degassed using vacuum/argon cycles (3×) and hydrogenated under balloon pressure overnight. The reaction mixture was filtered through celite and washed with EtOAc (30 mL). The solvent was removed under reduced pressure to afford the title compound as colourless oil (0.95 g, quantitative yield, rf=0.25 (10% MeOH in DCM)). The compound was used without further purification. MS: calculated for C₂₀H₃₄N₂O₁₁, 478.2. Found 479.4.

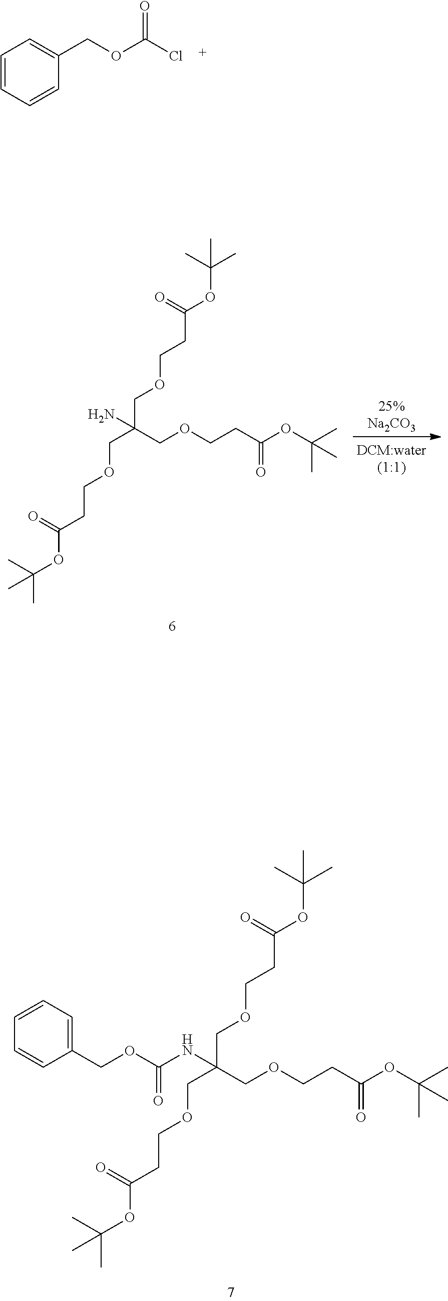 [SMILES: O=C(Cl)OCc1ccccc1 ; CC(C)(C)OC(=O)CCOCC(N)(COCCC(=O)OC(C)(C)C)COCCC(=O)OC(C)(C)C ; CC(C)(C)OC(=O)CCOCC(COCCC(=O)OC(C)(C)C)(COCCC(=O)OC(C)(C)C)NC(=O)OCc1ccccc1]

Preparation of compound 7: Tris{[2-(tert-butoxycarbonyl)ethoxy]methyl}-methylamine 6 (3.37 g, 6.67 mmol, 1.0 eq.) was dissolved in a mixture of DCM/water (40 mL 1:1 v/v) and Na₂CO₃ (0.18 g, 1.7 mmol, 0.25 eq.) was added while stirring vigorously. Benzyl chloroformate (2.94 mL, 20.7 mmol, 3.10 eq.) was added dropwise to the previous mixture and the reaction was stirred at room temperature for 24 h. The reaction mixture was diluted with CH₂Cl₂ (100 mL) and washed with water (100 mL). The organic layer was separated and dried over Na₂SO₄. The solvent was removed under reduced pressure and the resulting crude material was purified by flash chromatography (gradient elution: 0-10% EtOAc in cyclohexane in 12 CV) to afford the title compound as pale yellowish oil (3.9 g, 91%, rf=0.56 (10% EtOAc in cyclohexane)). MS: calculated for C₃₃H₅₃NO₁, 639.3. Found 640.9. ¹H NMR (500 MHz, DMSO-d₆) δ 7.38-7.26 (m, 5H), 4.97 (s, 2H), 3.54 (t, 6H), 3.50 (s, 6H), 2.38 (t, 6H), 1.39 (s, 27H). ¹³C NMR (125 MHz, DMSO-d₆) δ 170.3 (3×C), 154.5 (C), 137.1 (C), 128.2 (2×CH), 127.7 (CH), 127.6 (2×CH), 79.7 (3×C), 68.4 (3×CH₂), 66.8 (3×CH₂), 64.9 (C), 58.7 (CH₂), 35.8 (3×CH₂), 27.7 (9×CH₃).

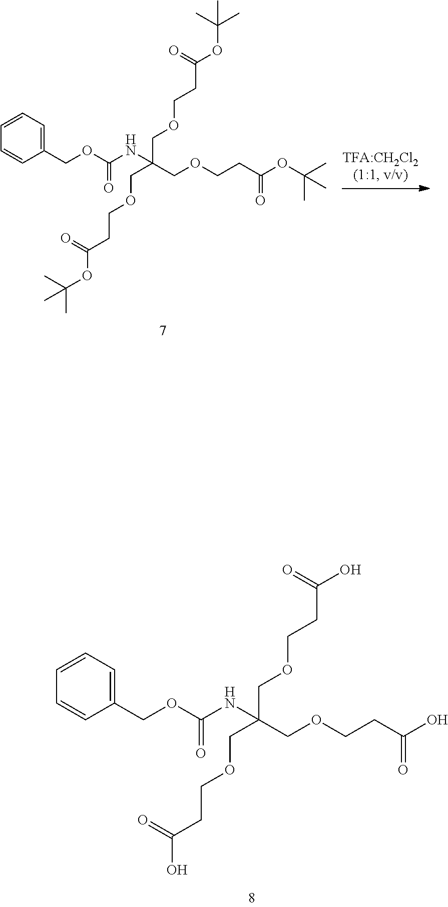 [SMILES: CC(C)(C)OC(=O)CCOCC(COCCC(=O)OC(C)(C)C)(COCCC(=O)OC(C)(C)C)NC(=O)OCc1ccccc1 ; O=C(O)CCOCC(COCCC(=O)O)(COCCC(=O)O)NC(=O)OCc1ccccc1]

Preparation of compound 8: Cbz-NH-tris-Boc-ester 7 (0.20 g, 0.39 mmol, 1.0 eq.) was dissolved in CH₂Cl₂ (1 mL) under argon, trifluoroacetic acid (TFA, 1 mL) was added and the reaction was stirred at room temperature for 1 h. The solvent was removed under reduced pressure, the residue was co-evaporated 3 times with toluene (5 mL) and dried under high vacuum to get the compound as its TFA salt (0.183 g, 98%). The compound was used without further purification. MS: calculated for C₂₁H₂₉NO₁₁, 471.6. Found 472.4.

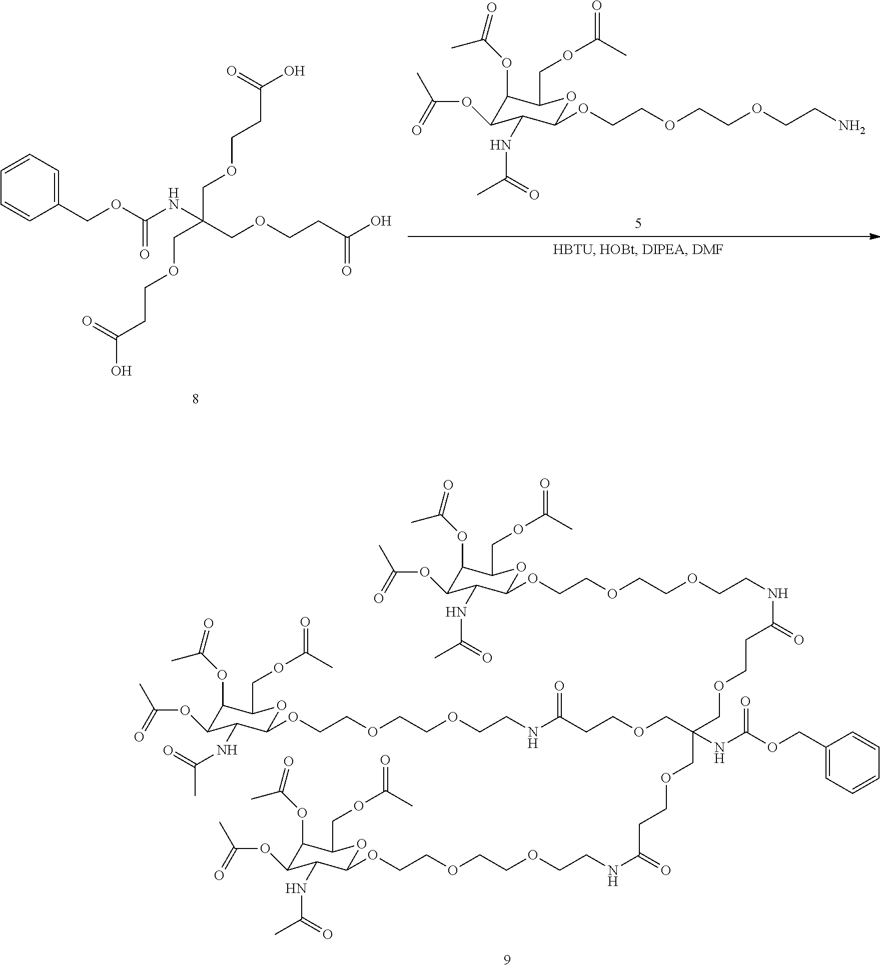 [SMILES: O=C(O)CCOCC(COCCC(=O)O)(COCCC(=O)O)NC(=O)OCc1ccccc1 ; CC(=O)NC1C(OCCOCCOCCN)OC(COC(C)=O)C(OC(C)=O)C1OC(C)=O ; CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)OCc2ccccc2)OC(COC(C)=O)C(OC(C)=O)C1OC(C)=O]

Preparation of compound 9: CbzNH-tris-COOH 8 (0.72 g, 1.49 mmol, 1.0 eq.) and GalNAc-PEG3-NH₂ 5 (3.56 g, 7.44 mmol, 5.0 eq.) were dissolved in N,N-dimethylformamide (DMF) (25 mL). Then N,N,N′,N′-tetramethyl-O-(1H-benzotriazol-1-yl)uroniumhexafluorophosphate (HBTU) (2.78 g, 7.44 mmol, 5.0 eq.), 1-hydroxybenzotriazole hydrate (HOBt) (1.05 g, 7.44 mmol, 5.0 eq.) and N,N-diisopropylethylamine (DIPEA) (2.07 mL, 11.9 mmol, 8.0 eq.) were added to the solution and the reaction was stirred for 72 h. The solvent was removed under reduced pressure, the residue was dissolved in DCM (100 mL) and washed with saturated aq. NaHCO₃ (100 mL). The organic layer was dried over Na₂SO₄, the solvent evaporated and the crude material was purified by flash chromatography (gradient elution: 0-5% MeOH in DCM in 14 CV). The product was obtained as pale yellowish oil (1.2 g, 43%, rf=0.20 (5% MeOH in DCM)). MS: calculated for C₈₁H₁₂₅N₇O₄₁, 1852.9. Found 1854.7. ¹H NMR (500 MHz, DMSO-d₆) δ 7.90-7.80 (m, 10H), 7.65-7.62 (m, 4H), 7.47-7.43 (m, 3H), 7.38-7.32 (m, 8H), 5.24-5.22 (m, 3H), 5.02-4.97 (m, 4H), 4.60-4.57 (m, 3H), 4.07-3.90 (m 10H), 3.67-3.36 (m, 70H), 3.23-3.07 (m, 25H), 2.18 (s, 10H), 2.00 (s, 13H), 1.89 (s, 11H), 1.80-1.78 (m, 17H). ¹³C NMR (125 MHz, DMSO-d₆) δ 170.1 (C), 169.8 (C), 169.7 (C), 169.4 (C), 169.2 (C), 169.1 (C), 142.7 (C), 126.3 (CH), 123.9 (CH), 118.7 (CH), 109.7 (CH), 100.8 (CH), 70.5 (CH), 69.8 (CH), 69.6 (CH), 69.5 (CH), 69.3 (CH₂), 69.0 (CH₂), 68.2 (CH₂), 67.2 (CH₂), 66.7 (CH₂), 61.4 (CH₂), 22.6 (CH₂), 22.4 (3×CH₃), 20.7 (9×CH₃).

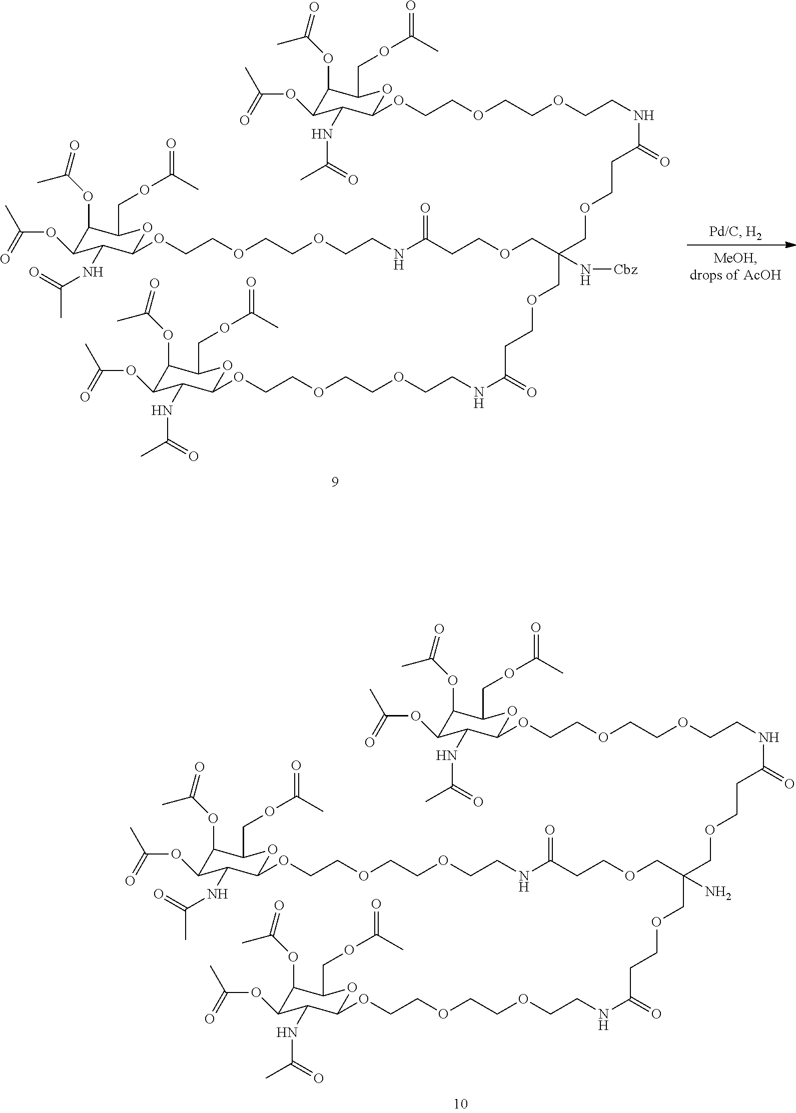 [SMILES: CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)OCc2ccccc2)OC(COC(C)=O)C(OC(C)=O)C1OC(C)=O ; CC(=O)NC1C(OCCOCCOCCNC(=O)CCOCC(N)(COCCC(=O)NCCOCCOCCOC2OC(COC(C)=O)C(OC(C)=O)C(OC(C)=O)C2NC(C)=O)COCCC(=O)NCCOCCOCCOC2OC(COC(C)=O)C(OC(C)=O)C(OC(C)=O)C2NC(C)=O)OC(COC(C)=O)C(OC(C)=O)C1OC(C)=O]

Preparation of compound 10: Triantennary GalNAc compound 9 (0.27 g, 0.14 mmol, 1.0 eq.) was dissolved in MeOH (15 mL), 3 drops of acetic acid (AcOH) and Pd/C (30 mg) was added. The reaction mixture was degassed using vacuum/argon cycles (3×) and hydrogenated under balloon pressure overnight. The completion of the reaction was followed by mass spectrometry and the resulting mixture was filtered through a thin pad of celite. The solvent was evaporated and the residue obtained was dried under high vacuum and used for the next step without further purification. The product was obtained as pale yellowish oil (0.24 g, quantitative yield). MS: calculated for C₇₃H₁₁₉N₇O₃₉, 1718.8. Found 1719.3.

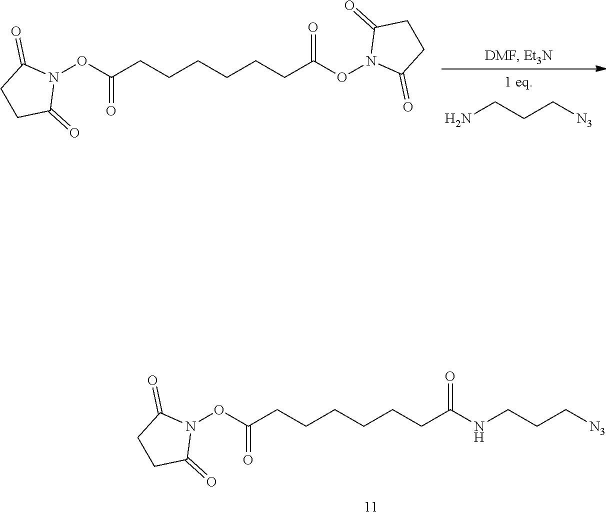 [SMILES: O=C(CCCCCCC(=O)ON1C(=O)CCC1=O)ON1C(=O)CCC1=O ; [N-]=[N+]=NCCCN ; [N-]=[N+]=NCCCNC(=O)CCCCCCC(=O)ON1C(=O)CCC1=O]

Preparation of compound 11: Commercially available suberic acid bis(N-hydroxysuccinimide ester) (3.67 g, 9.9 mmol, 1.0 eq.) was dissolved in DMF (5 mL) and triethylamine (1.2 mL) was added. To this solution was added dropwise a solution of 3-azido-1-propylamine (1.0 g, 9.9 mmol, 1.0 eq.) in DMF (5 mL). The reaction was stirred at room temperature for 3 h. The reaction mixture was diluted with EtOAc (100 mL) and washed with water (50 mL). The organic layer was separated, dried over Na₂SO₄ and the solvent was removed under reduced pressure. The crude material was purified by flash chromatography (gradient elution: 0-5% MeOH in DCM in 16 CV). The product was obtained as white solid (1.54 g, 43%, rf=0.71 (5% MeOH in DCM)). MS: calculated for C₁₅H₂₃N₅O₅, 353.4. Found 354.3.

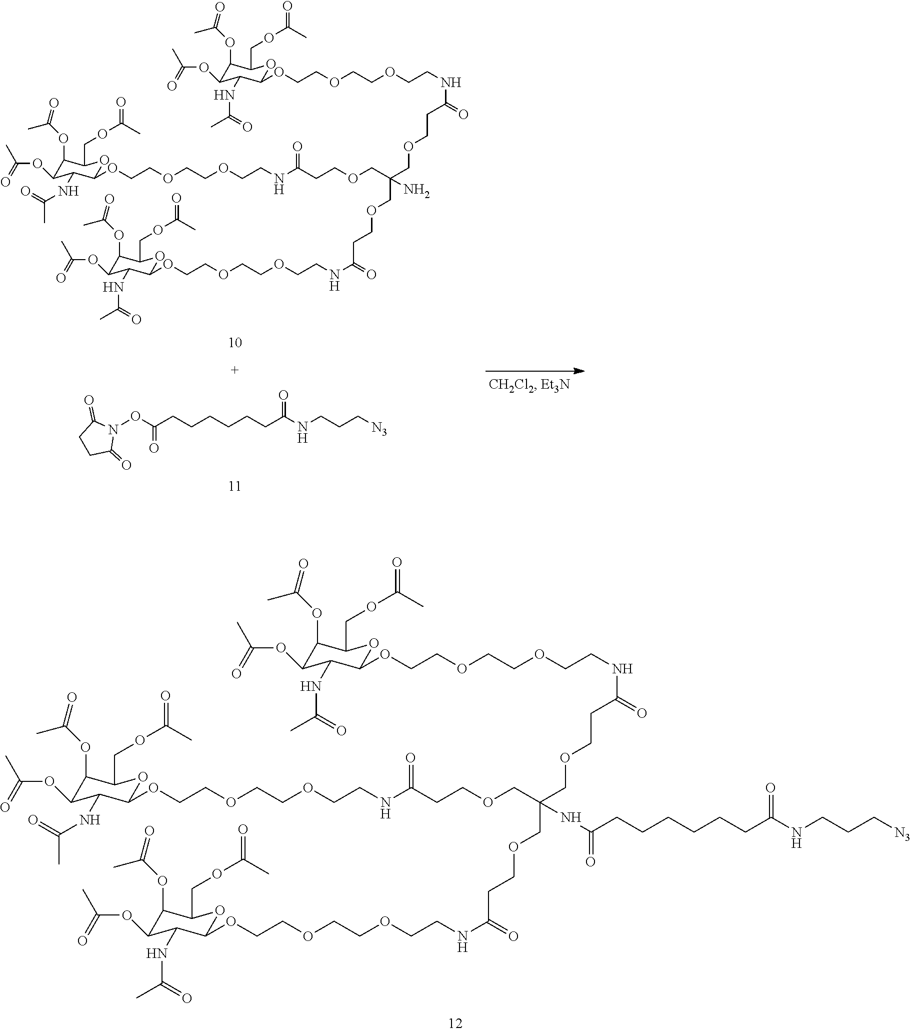 [SMILES: CC(=O)NC1C(OCCOCCOCCNC(=O)CCOCC(N)(COCCC(=O)NCCOCCOCCOC2OC(COC(C)=O)C(OC(C)=O)C(OC(C)=O)C2NC(C)=O)COCCC(=O)NCCOCCOCCOC2OC(COC(C)=O)C(OC(C)=O)C(OC(C)=O)C2NC(C)=O)OC(COC(C)=O)C(OC(C)=O)C1OC(C)=O ; [N-]=[N+]=NCCCNC(=O)CCCCCCC(=O)ON1C(=O)CCC1=O ; CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)CCCCCCC(=O)NCCCN=[N+]=[N-])OC(COC(C)=O)C(OC(C)=O)C1OC(C)=O]

Preparation of TriGalNAc (12): Triantennary GalNAc compound 10 (0.35 g, 0.24 mmol, 1.0 eq.) and compound 11 (0.11 g, 0.31 mmol, 1.5 eq.) were dissolved in DCM (5 mL) under argon and triethylamine (0.1 mL, 0.61 mmol, 3.0 eq.) was added. The reaction was stirred at room temperature overnight. The solvent was removed under reduced pressure, the residue was dissolved in EtOAc (100 mL) and washed with water (100 mL). The organic layer was separated and dried over Na₂SO₄. The solvent was evaporated and the resulting crude material was purified by flash chromatography (elution gradient: 0-10% MeOH in DCM in 20 CV) to afford the title compound as white fluffy solid (0.27 g, 67%, rf=0.5 (10% MeOH in DCM)). MS: calculated for C₈₄H₁₃₇N₁₁O₄₁, 1957.1. Found 1959.6.

Compound 12 was used for subsequent oligonucleoside conjugate preparations employing “click chemistry”.

iii) Oligonucleoside Synthesis

TABLE 8

| Single | | Purity by RP |
|---|---|---|
| strand ID | Sequence 5′ - 3′ | HPLC (%) |
| X91382 | (NH2- | 89.5 |
| | DEG)gacuuuCfaUfCfCfuggaaauasusa(invabasic)(invabasic) | |
| X91383 | (NH2- | 91.6 |
| | DEG)aaGfcAfaGfaUfAfUfuUfuuAfuAfasusa(invabasic) | |
| | (invabasic) | |
| X91384 | (NH2- | 94.0 |
| | DEG)ugggauUfuCfAfUfguaaccaasgsa(invabasic)(invabasic) | |
| X91403 | (NH2C12)gacuuuCfaUfCfCfuggaaauasusa(invabasic) | 94.2 |
| | (invabasic) | |
| X91404 | (NH2C12)aaGfcAfaGfaUfAfUfuUfuuAfuAfasusa(invabasic) | 96.5 |
| | (invabasic) | |
| X91405 | (NH2C12)ugggauUfuCfAfUfguaaccaasgsa(invabasic) | 91.3 |
| | (invabasic) | |
| X91415 | (invabasic)(invabasic)gsascuuuCfaUfCfCfuggaaauasusa | 96.4 |
| | (NH2C6) | |
| X91416 | (invabasic)(invabasic)asasGfcAfaGfaUfAfUfuUfuuAfuAfaua | 77.4 |
| | (NH2C6) | |
| X91417 | (invabasic)(invabasic)usgsggauUfuCfAfUfguaaccaaga | 96.7 |
| | (NH2C6) | |
| X91379 | gsascuuuCfaUfCfCfuggaaauaua(GalNAc) | 92.8 |
| X91380 | asasGfcAfaGfaUfAfUfuUfuuAfuAfaua(GalNAc) | 95.7 |
| X91446 | usgsggauUfuCfAfUfguaaccaaga(GalNAc) | 92.1 |
| X38483 | usAfsuauUfuCfCfaggaUfgAfaagucscsa | 91.0 |
| X91381 | usAfsUfuAfuaAfaAfauaUfcUfuGfcuususudTdT | 90.0 |
| X38104 | usCfsuugGfuuAfcaugAfaAfucccasusc | 95.4 |

Af, Cf, Gf, Uf. 2′-F RNA nucleosides
a, c, g, u: 2′-O-Me RNA nucleosides
dT: DNA nucleosides
s: Phosphorothioate
invabasic: 1,2-dideoxyribose
NH2-DEG: Aminoethoxyethyl linker
NH2C12: Aminododecyl linker
NH2C6: Aminohexyl linker

Oligonucleosides were synthesized on solid phase according to the phosphoramidite approach. Depending on the scale either a Mermade 12 (BioAutomation Corporation) or an AKTA Oligopilot (GE Healthcare) was used.

Syntheses were performed on commercially available solid supports made of controlled pore glass either loaded with invabasic (CPG, 480 Å, with a loading of 86 μmol/g; LGC Biosearch cat. #BCG-1047-B) or 2′-F A (CPG, 520 Å, with a loading of 90 μmol/g; LGC Biosearch cat. #BCG-1039-B) or NH2C6 (CPG, 520 Å, with a loading of 85 μmol/g LGC Biosearch cat. #BCG-1397-B) or GalNAc (CPG, 500 Å, with a loading of 57 μmol/g; Primetech) or 2′-O-Methyl C (CPG, 500 Å, with a loading of 84 μmol/g LGC Biosearch cat. #BCG-10-B) or 2′-O-Methyl A (CPG, 497 Å, with a loading of 85 mol/g, LGC Biosearch, Cat. #BCG-1029-B) or dT (CPG, 497 Å, with a loading of 87 μmol/g LGC Biosearch, cat. #BCG-1055-B).

2′-O-Me, 2′-F RNA phosphoramidites and ancillary reagents were purchased from SAFC Proligo (Hamburg, Germany).

2′-O-Methyl phosphoramidites include: 5′-(4,4′-dimethoxytrityl)-N-benzoyl-adenosine 2′-O-methyl-3′-[(2-cyanoethyl)-(N,N-diisopropyl)]-phosphoramidite, 5′-(4,4′-dimethoxytrityl)-N-benzoyl-cytidine 2′-O-methyl-3′-[(2-cyanoethyl)-(N,N-diisopropyl)]-phosphoramidite, 5′-(4,4′-dimethoxytrityl)-N-dimethylformamidine-guanosine 2′-O-methyl-3′-[(2-cyanoethyl)-(N,N-diisopropyl)]-phosphoramidite, 5′-(4,4′-dimethoxytrityl)-uridine 2′-O-methyl-3′-[(2-cyanoethyl)-(N,N-diisopropyl)]-phosphoramidite.

2′-F phosphoramidites include: 5′-dimethoxytrityl-N-benzoyl-deoxyadenosine 2-fluoro-3′-[(2-cyanoethyl)-(N,N-diisopropyl)]-phosphoramidite, 5′-dimethoxytrityl-N-acetyl-deoxycytidine 2′-fluoro-3′-[(2-cyanoethyl)-(N,N-diisopropyl)]-phosphoramidite, 5′-dimethoxytrityl-N-isobutyryl-deoxyguanosine 2′-fluoro-3′-[(2-cyanoethyl)-(N,N-diisopropyl)]-phosphoramidite and 5′-dimethoxytrityl-deoxyuridine 2′-fluoro-3′-[(2-cyanoethyl)-(N,N-diisopropyl)]-phosphoramidite.

In order to introduce the required amino linkers at the 5′-end of the oligonucleosides the 2-[2-(4-Monomethoxytrityl)aminoethoxy]ethyl-(2-cyanoethyl)-N,N-diisopropyl)-phosphoramidite (Glen Research Cat. #1905) and the 12-(trifluoroacetylamino)dodecyl-[(2-cyanoethyl)-(N,N-diisopropyl)]-phosphoramidite (ChemGenes Cat. #CLP-1575) were employed. The invabasic modification was introduced using 5-O-dimethoxytrityl-1,2-dideoxyribose-3-[(2-cyanoethyl)-(N,N-diisopropyl)]-phosphoramidite (ChemGenes Cat. #ANP-1422).

All building blocks were dissolved in anhydrous acetonitrile (100 mM (Mermade12) or 200 mM (AKTA Oligopilot)) containing molecular sieves (3 Å) except 2′-O-methyl-uridine phosphoramidite which was dissolved in 50% anhydrous DCM in anhydrous acetonitrile. Iodine (50 mM in pyridine/H₂O 9:1 v/v) was used as oxidizing reagent. 5-Ethyl thiotetrazole (ETT, 500 mM in acetonitrile) was used as activator solution.

Thiolation for introduction of phosphorthioate linkages was carried out using 100 mM xanthane hydride (TCI, Cat. #6846-35-1) in acetonitrile/pyridine 4:6 v/v.

Coupling times were 5.4 minutes except when stated otherwise. 5′ amino modifications were incorporated into the sequence employing a double coupling step with a coupling time of 11 minutes per each coupling (total coupling time 22 min). The oxidizer contact time was set to 1.2 min and thiolation time was 5.2 min.

Sequences were synthesized with removal of the final DMT group, with exception of the MMT group from the NH2DEG sequences.

At the end of the synthesis, the oligonucleosides were cleaved from the solid support using a 1:1 volume solution of 28-30% ammonium hydroxide (Sigma-Aldrich, Cat. #221228) and 40% aqueous methylamine (Sigma-Aldrich, Cat. #8220911000) for 16 hours at 6° C. The solid support was then filtered off, the filter was thoroughly washed with H₂O and the volume of the combined solution was reduced by evaporation under reduced pressure. The pH of the resulting solution was adjusted to pH 7 with 10% AcOH (Sigma-Aldrich, Cat. #A6283).

The crude materials were purified either by reversed phase (RP) HPLC or anion exchange (AEX) HPLC.

RP HPLC purification was performed using a XBridge C18 Prep 19×50 mm column (Waters) on an AKTA Pure instrument (GE Healthcare). Buffer A was 100 mM triethyl-ammonium acetate (TEAAc, Biosolve) pH 7 and buffer B contained 95% acetonitrile in buffer A. A flow rate of 10 m/min and a temperature of 60° C. were employed. UV traces at 280 nm were recorded. A gradient of 0% B to 100% B within 120 column volumes was employed. Appropriate fractions were pooled and precipitated in the freezer with 3 M sodium acetate (NaOAc) (Sigma-Aldrich), pH 5.2 and 85% ethanol (VWR). Pellets were isolated by centrifugation, redissolved in water (50 mL), treated with 5 M NaCl (5 mL) and desalted by Size exclusion HPLC on an Akta Pure instrument using a 50×165 mm ECO column (YMC, Dinslaken, Germany) filled with Sephadex G25-Fine resin (GE Healthcare).

AEX HPLC purification was performed using a TSK gel SuperQ-5PW 20×200 mm (BISCHOFF Chromatography) on an AKTA Pure instrument (GE Healthcare). Buffer A was 20 mM sodium phosphate (Sigma-Aldrich) pH 7.8 and buffer B was the same as buffer A with the addition of 1.4 M sodium bromide (Sigma-Aldrich). A flow rate of 10 mL/min and a temperature of 60° C. were employed. UV traces at 280 nm were recorded. A gradient of 10% B to 100% B within 27 column volumes was employed. Appropriate fractions were pooled and precipitated in the freezer with 3 M NaOAc, pH 5.2 and 85% ethanol. Pellets were isolated by centrifugation, redissolved in water (50 mL), treated with 5 M NaCl (5 mL) and desalted by size exclusion chromatography.

The MMT group was removed with 25% acetic acid in water. Once the reaction was complete the solution was neutralized and the samples were desalted by size exclusion chromatography.

Single strands were analyzed by analytical LC-MS on a 2.1×50 mm XBridge C18 column (Waters) on a Dionex Ultimate 3000 (Thermo Fisher Scientific) HPLC system combined either with a LCQ Deca XP-plus Q-ESI-TOF mass spectrometer (Thermo Finnigan) or with a Compact ESI-Qq-TOF mass spectrometer (Bruker Daltonics). Buffer A was 16.3 mM triethylamine, 100 mM 1,1,1,3,3,3-hexafluoro-2-propanol (HFIP) and 1% MeOH in H₂O and buffer B contained buffer A in 95% MeOH. A flow rate of 250 μL/min and a temperature of 60° C. were employed. UV traces at 260 and 280 nm were recorded. A gradient of 1-40% B within 0.5 min followed by 40 to 100% B within 13 min was employed. Methanol (LC-MS grade), water (LC-MS grade), 1,1,1,3,3,3-hexafluoro-2-propanol (puriss. p.a.) and triethylamine (puriss. p.a.) were purchased from Sigma-Aldrich.

iv) Monofluoro cyclooctyne (MFCO) conjugation at 5′- or 3′-end

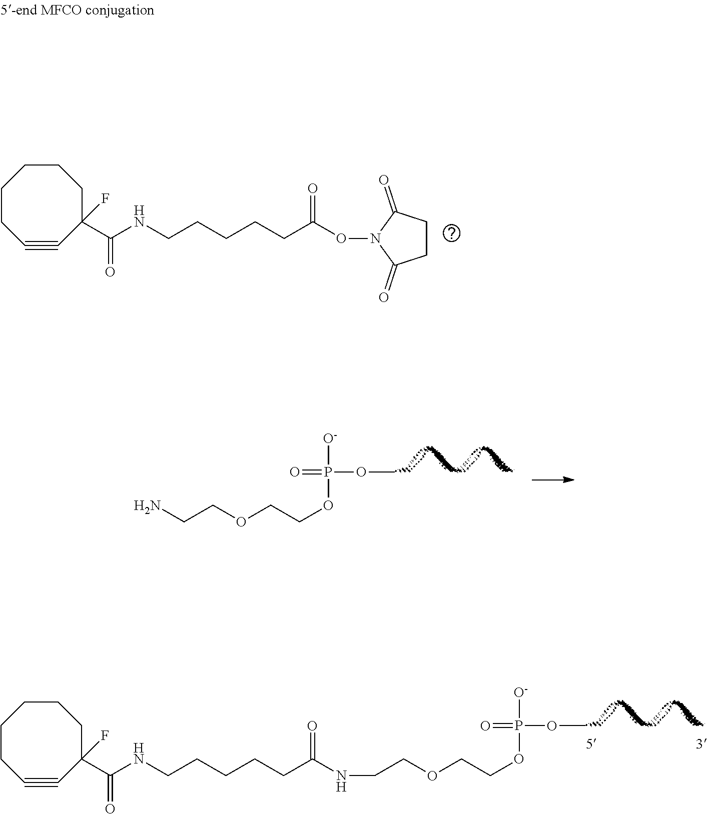 [SMILES: O=C(CCCCCNC(=O)C1(F)C#CCCCCC1)ON1C(=O)CCC1=O ; COP(=O)([O-])OCCOCCN ; COP(=O)([O-])OCCOCCNC(=O)CCCCCNC(=O)C1(F)C#CCCCCC1]

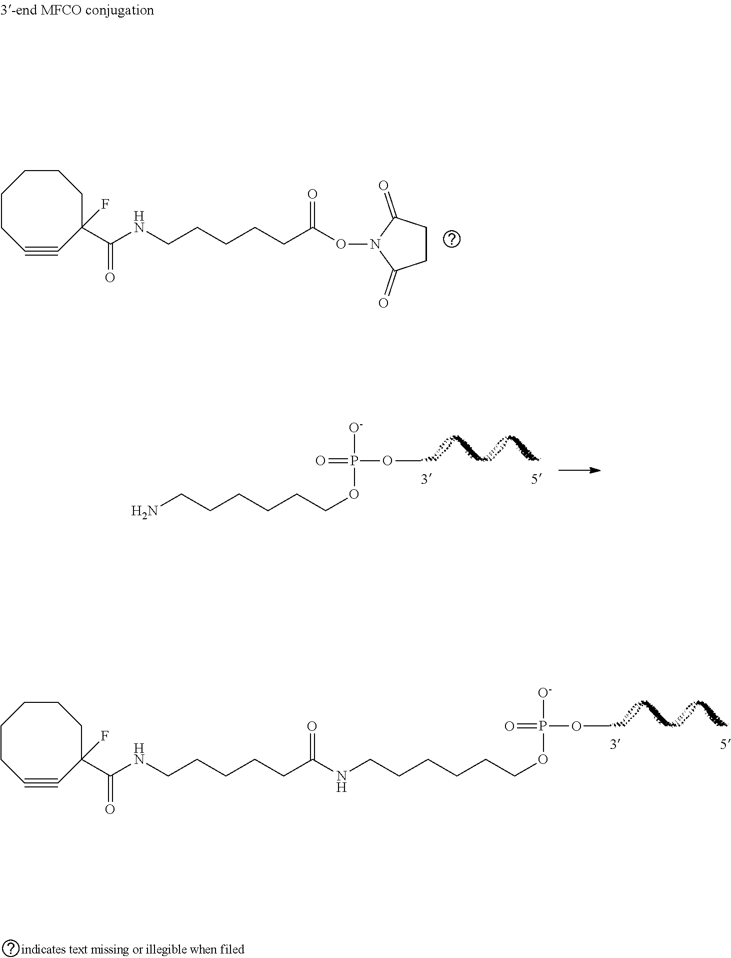 [SMILES: O=C(CCCCCNC(=O)C1(F)C#CCCCCC1)ON1C(=O)CCC1=O ; COP(=O)([O-])OCCCCCCN ; COP(=O)([O-])OCCCCCCNC(=O)CCCCCNC(=O)C1(F)C#CCCCCC1]

General conditions for MFCO conjugation: Amine-modified single strand was dissolved at 700 OD/mL in 50 mM carbonate/bicarbonate buffer pH 9.6/dimethyl sulfoxide (DMSO) 4:6 (v/v) and to this solution was added one molar equivalent of a 35 mM solution of MFCO-C6-NHS ester (Berry & Associates, Cat. #LK 4300) in DMF. The reaction was carried out at room temperature and after 1 h another molar equivalent of the MFCO solution was added. The reaction was allowed to proceed for an additional hour and was monitored by LC/MS. At least two molar equivalent excess of the MFCO NHS ester reagent relative to the amino modified oligonucleoside were needed to achieve quantitative consumption of the starting material. The reaction mixture was diluted 15-fold with water, filtered trough a 1.2 μm filter from Sartorius and then purified by reserve phase (RP HPLC) on an Akta Pure instrument (GE Healthcare).

Purification was performed using a XBridge C18 Prep 19×50 mm column from Waters. Buffer A was 100 mM TEAAc pH 7 and buffer B contained 95% acetonitrile in buffer A. A flow rate of 10 mL/min and a temperature of 60° C. were employed. UV traces at 280 nm were recorded. A gradient of 0-100% B within 60 column volumes was employed.

Fractions containing full length conjugated oligonucleoside were pooled, precipitated in the freezer with 3 M NaOAc, pH 5.2 and 85% ethanol and the collected pellet was dissolved in water. Samples were desalted by size exclusion chromatography and concentrated using a speed-vac concentrator to yield the conjugated oligonucleoside in an isolated yield of 40-80%.

TABLE 9

| Sense | | Purity by RP |
|---|---|---|
| strand ID | Sense strand sequence 5′ - 3′ | HPLC (%) |
| X91388 | (MFCO)(NH- | 89.0 |
| | DEG)gacuuuCfaUfCfCfuggaaauasusa(invabasic)(invabasic) | |
| X91389 | (MFCO)(NH- | 91.0 |
| | DEG)aaGfcAfaGfaUfAfUfuUfuuAfuAfasusa(invabasic) | |
| | (invabasic) | |
| X91390 | (MFCO)(NH- | 90.0 |
| | DEG)ugggauUfuCfAfUfguaaccaasgsa(invabasic)(invabasic) | |
| X91421 | (invabasic)(invabasic)gsascuuuCfaUfCfCfuggaaauasusa(NHC6) | 94.0 |
| | (MFCO) | |
| X91422 | (invabasic)(invabasic)asasGfcAfaGfaUfAfUfuUfuuAfuAfauac | 89.0 |
| | (NH2C6C6)(MFCO) | |
| X91423 | (invabasic)(invabasic)usgsggau UfuCfAfUfguaaccaaga(NHC6) | 89.0 |
| | (MFCO) | |

v) TriGalNAc (GalNAc-T1) conjugation at 5′- or 3′-end

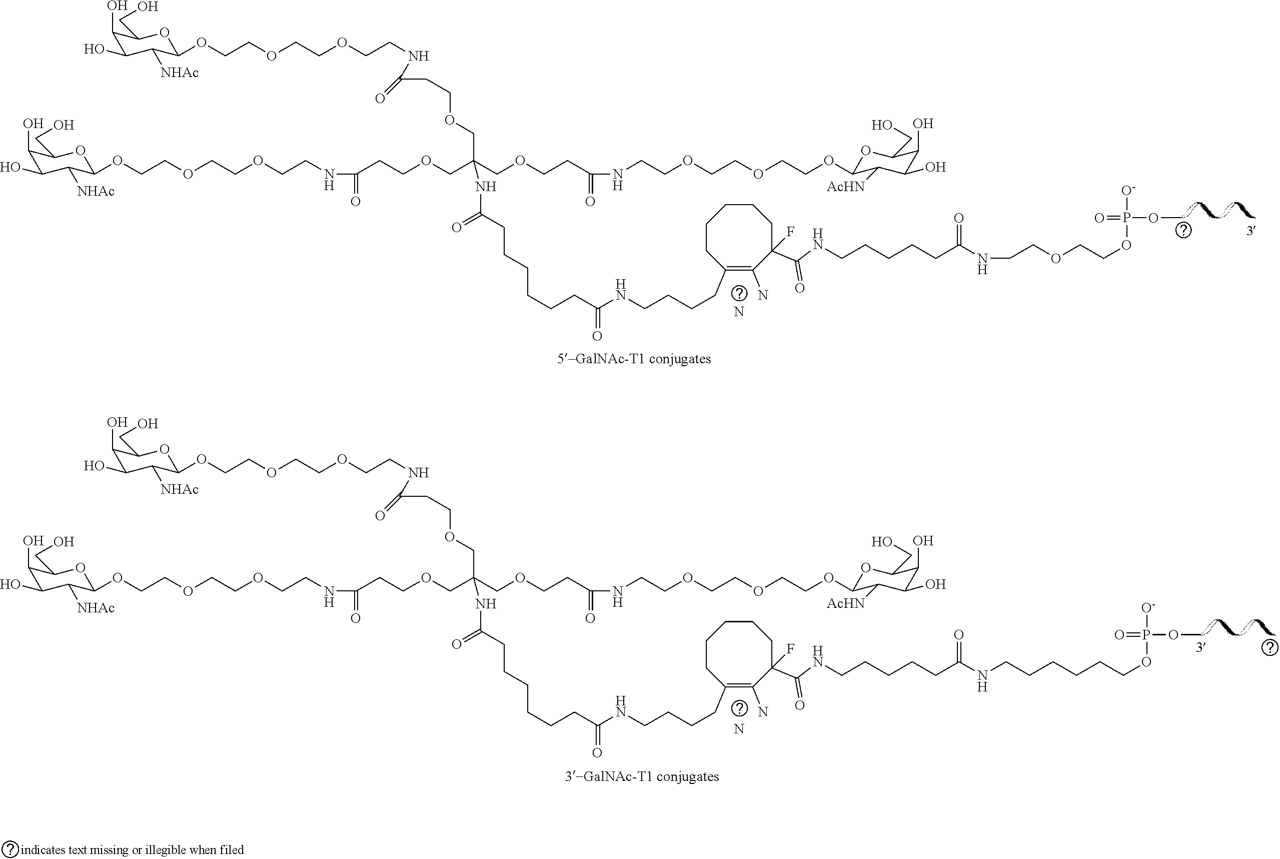

General procedure for TriGalNAc conjugation: MFCO-modified single strand was dissolved at 2000 OD/mL in water and to this solution was added one equivalent solution of compound 12 (10 mM) in DMF. The reaction was carried out at room temperature and after 3 h 0.7 molar equivalent of the compound 12 solution was added. The reaction was allowed to proceed overnight and completion was monitored by LCMS. The conjugate was diluted 15-fold in water, filtered through a 1.2 μm filter from Sartorius and then purified by RP HPLC on an Akta Pure instrument (GE Healthcare).

RP HPLC purification was performed using a XBridge C18 Prep 19×50 mm column from Waters. Buffer A was 100 mM triethylammonium acetate pH 7 and buffer B contained 95% acetonitrile in buffer A. A flow rate of 10 m/min and a temperature of 60° C. were employed. UV traces at 280 nm were recorded. A gradient of 0-100% B within 60 column volumes was employed.

Fractions containing full-length conjugated oligonucleoside were pooled, precipitated in the freezer with 3 M NaOAc, pH 5.2 and 85% ethanol and the collected pellet was dissolved in water to give an oligonucleoside solution of about 1000 OD/mL. The O-acetates were removed by adding 20% aqueous ammonia. Quantitative removal of these protecting groups was verified by LC-MS.

The conjugates were desalted by size exclusion chromatography using Sephadex G25 Fine resin (GE Healthcare) on an Akta Pure (GE Healthcare) instrument to yield the conjugated nucleoside in an isolated yield of 50-70%.

TABLE 10

| Sense | | Purity by |
|---|---|---|
| strand | | RP HPLC |
| ID | Sense strand sequence 5′ - 3′ | (%) |
| X91394 | (GalNAc-T1)(MFCO)(NH- | 80.0 |
| | DEG)gacuuuCfaUfCfCfuggaaauasusa(invabasic)(invabasic) | |
| X91395 | (GalNAc-T1)(MFCO)(NH- | 87.8 |
| | DEG)aaGfcAfaGfaUfAfUfuUfuuAfuAfasusa(invabasic)(invabasic) | |
| X91396 | (GalNAc-T1)(MFCO)(NH- | 87.9 |
| | DEG)ugggauUfuCfAfUfguaaccaasgsa(invabasic)(invabasic) | |
| X91427 | (invabasic)(invabasic)gsascuuuCfaUfCfCfuggaaauasusa | 88.0 |
| | (NHC6)(MFCO)(GalNAc-T1) | |
| X91428 | (invabasic)(invabasic)asasGfcAfaGfaUfAfUfuUfuuAfuAfaua | 82.6 |
| | (NHC6)(MFCO)(GalNAc-T1) | |
| X91429 | (invabasic)(invabasic)usgsggauUfuCfAfUfguaaccaaga(NHC6) | 82.9 |
| | (MFCO)(GalNAc-T1) | |

### vi) Duplex Annealing

To generate the desired siRNA duplex, the two complementary strands were annealed by combining equimolar aqueous solutions of both strands. The mixtures were placed into a water bath at 70° C. for 5 minutes and subsequently allowed to cool to ambient temperature within 2 h. The duplexes were lyophilized for 2 days and stored at −20° C.

The duplexes were analyzed by analytical SEC HPLC on Superdex™ 75 Increase 5/150 GL column 5×153-158 mm (Cytiva) on a Dionex Ultimate 3000 (Thermo Fisher Scientific) HPLC system. Mobile phase consisted of 1×PBS containing 10% acetonitrile. An isocratic gradient was run in 10 min at a flow rate of 1.5 mL/min at room temperature. UV traces at 260 and 280 nm were recorded. Water (LC-MS grade) was purchased from Sigma-Aldrich and Phosphate-buffered saline (PBS; 10×, pH 7.4) was purchased from GIBCO (Thermo Fisher Scientific).

GalNAc conjugates prepared are compiled in the table below. These were directed against 3 different target genes. siRNA coding along with the corresponding single strands, sequence information as well as purity for the duplexes is captured.

TABLE 11

| | | | | Duplex |
|---|---|---|---|---|
| | Duplex | | | Purity by |
| Target | ID | SSRN ID | ssRNA-Sequence 5′-3′ | HPLC (%) |
| GO | ETX001 | X91394 | (GalNAc- | 96.8 |
| | | | T1)(MFCO)(NHDEG)gacuuuCfaUfCfCfugg | |
| | | | aaauasusa(invabasic)(invabasic) | |
| | | X38483 | usAfsuauUfuCfCfaggaUfgAfaagucscsa | |
| | ETX005 | X91427 | (invabasic)(invabasic)gsascuuuCfaUfCfCfug | 92.8 |
| | | | gaaauasusa(NHC6)(MFCO)(GalNAc-T1) | |
| | | X38483 | usAfsuauUfuCfCfaggaUfgAfaagucscsa | |
| C5 | ETX010 | X91395 | (GalNAc-T1)(MFCO)(NH- | 96.4 |
| | | | DEG)aaGfcAfaGfaUfAfUfuUfuuAfuAfasus | |
| | | | a(invabasic)(invabasic) | |
| | | X91381 | usAfsUfuAfuaAfaAfauaUfcUfuGfcuususud | |
| | | | TdT | |
| | ETX014 | X91428 | (invabasic)(invabasic)asasGfcAfaGfaUfAfU | 97.2 |
| | | | fuUfuuAfuAfaua(NHC6)(MFCO)(GalNAc- | |
| | | | T1) | |
| | | X91381 | usAfsUfuAfuaAfaAfauaUfcUfuGfcuususud | |
| | | | TdT | |
| TTR | ETX019 | X91396 | (GalNAc-T1)(MFCO)(NH- | 97.2 |
| | | | DEG)ugggauUfuCfAfUfguaaccaasgsa | |
| | | | (invabasic)(invabasic) | |
| | | X38104 | usCfsuugGfuu AfcaugAfaAfucccasusc | |
| | ETX023 | X91429 | (invabasic)(invabasic)usgsggauUfuCfAfUfg | 96.3 |
| | | | uaaccaaga(NHC6)(MFCO)(GalNAc-T1) | |
| | | X38104 | usCfsuugGfuu AfcaugAfaAfucccasusc | |

The following schemes further set out the routes of synthesis:

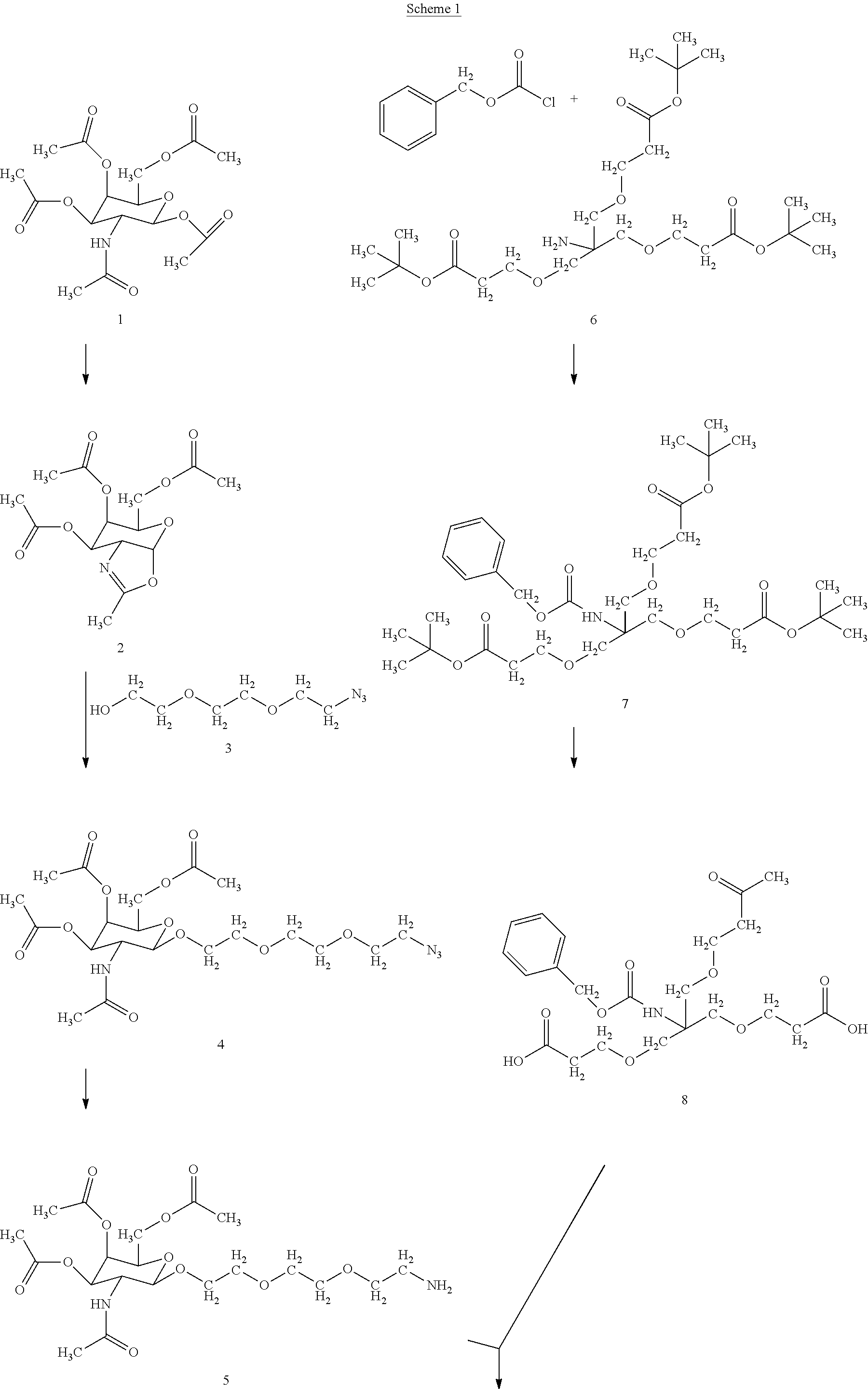

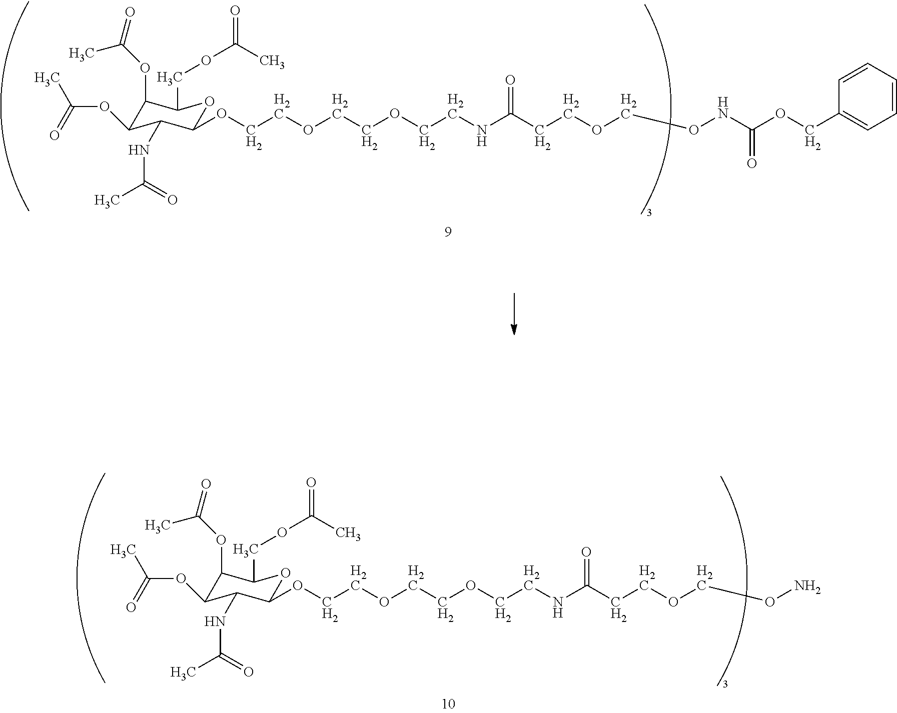

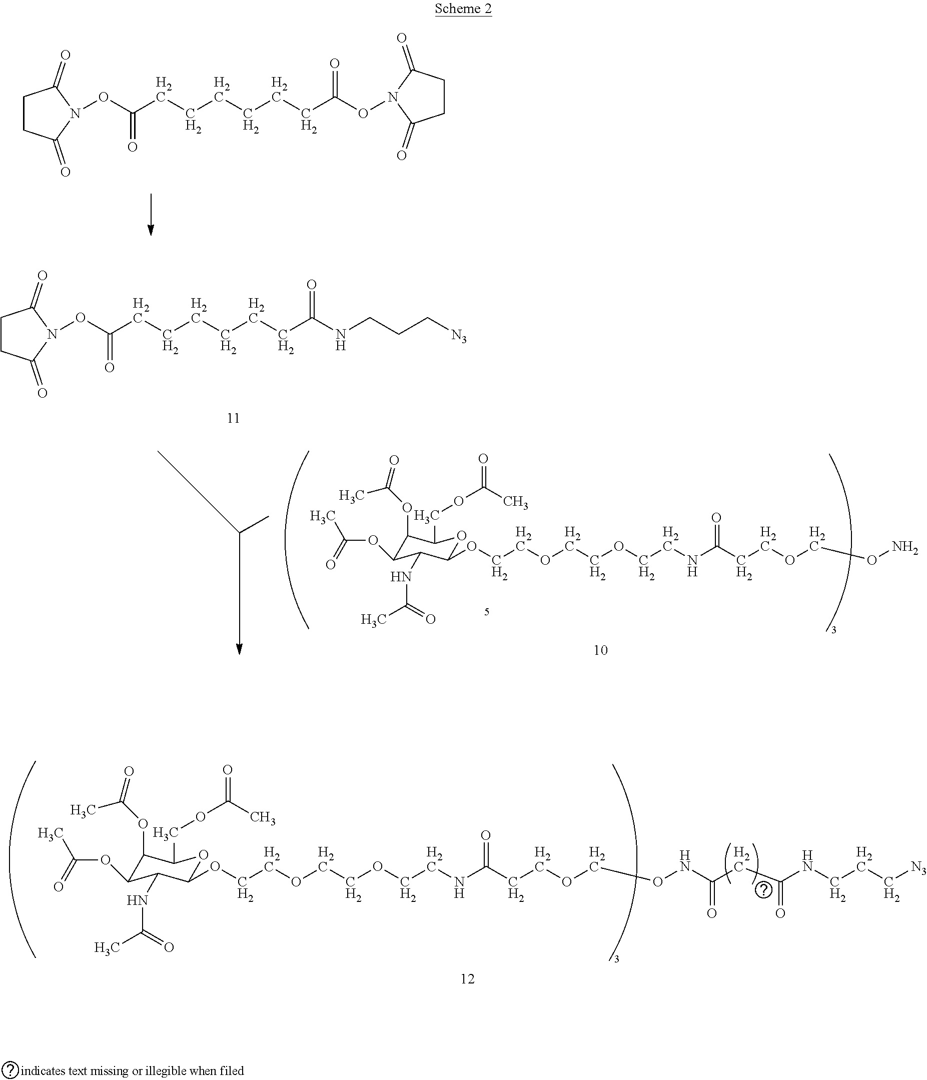

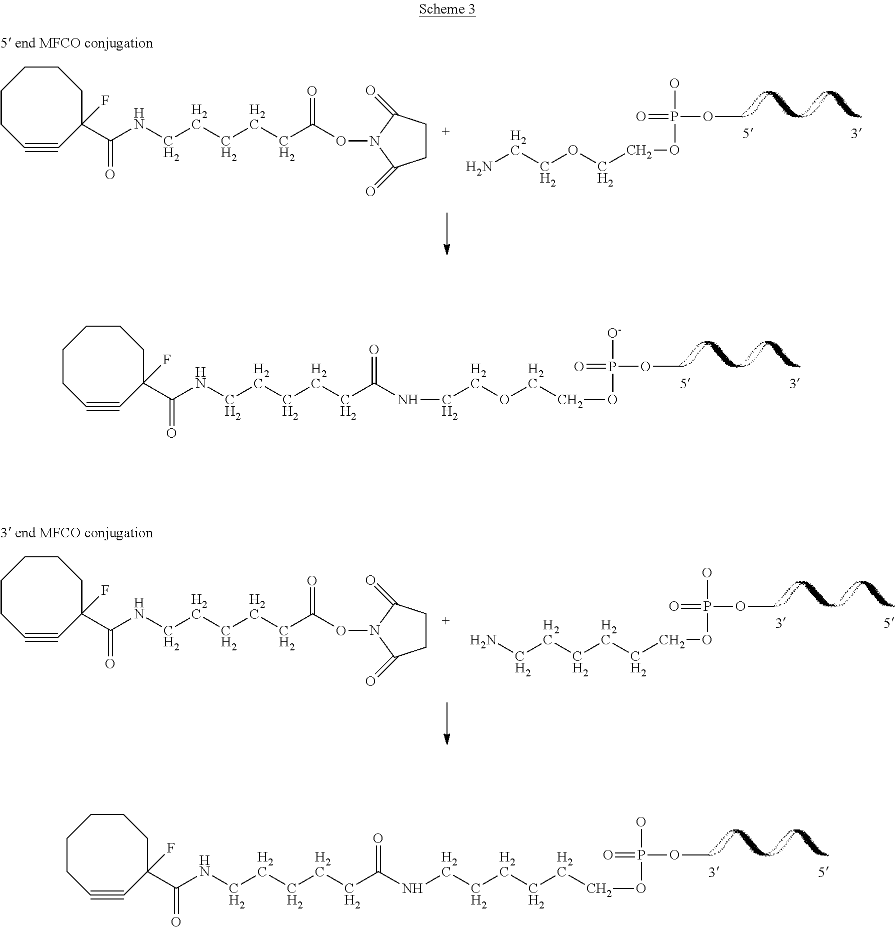 [SMILES: O=C(CCCCCNC(=O)C1(F)C#CCCCCC1)ON1C(=O)CCC1=O ; COP(=O)(O)OCCOCCN ; COP(=O)([O-])OCCOCCNC(=O)CCCCCNC(=O)C1(F)C#CCCCCC1 ; O=C(CCCCCNC(=O)C1(F)C#CCCCCC1)ON1C(=O)CCC1=O ; COP(=O)(O)OCCCCCCN ; COP(=O)(O)OCCCCCCNC(=O)CCCCCNC(=O)C1(F)C#CCCCCC1]

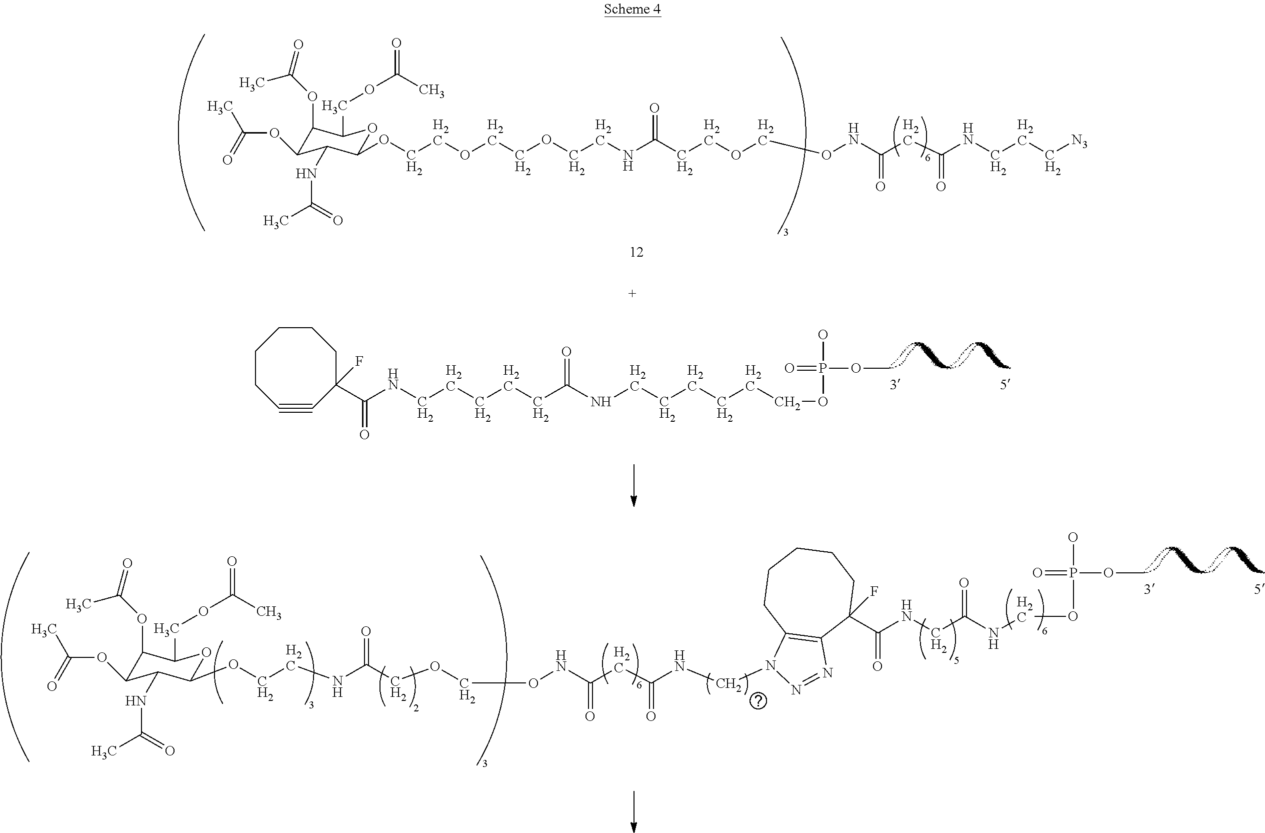

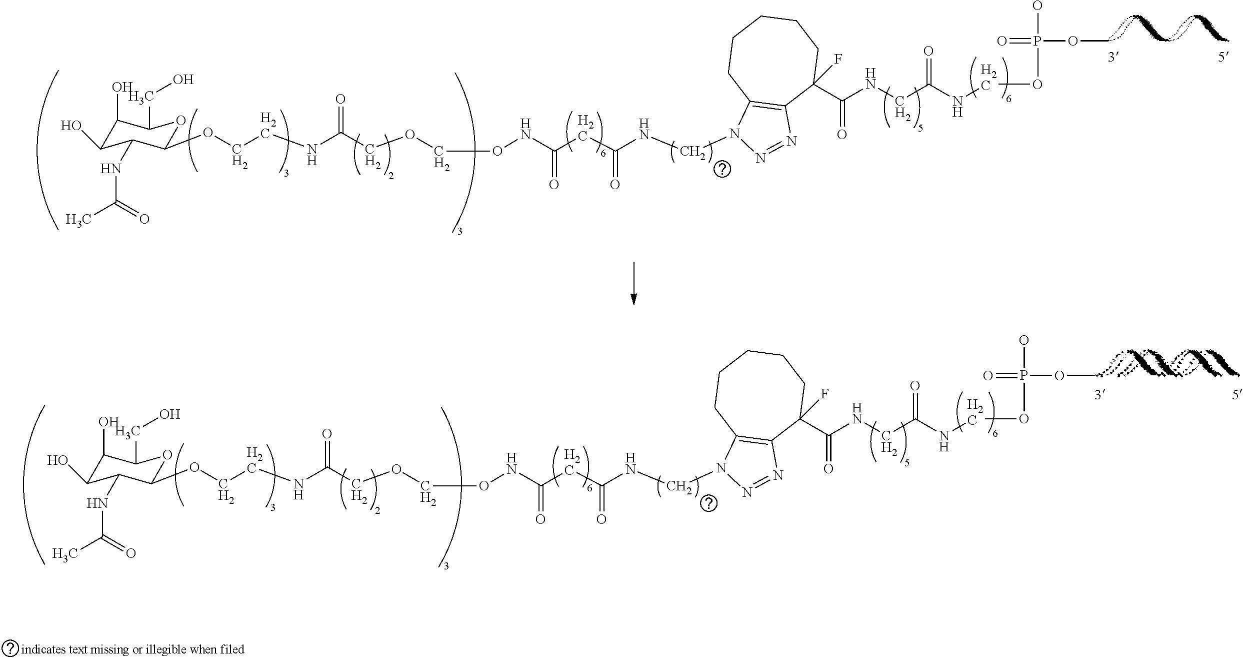

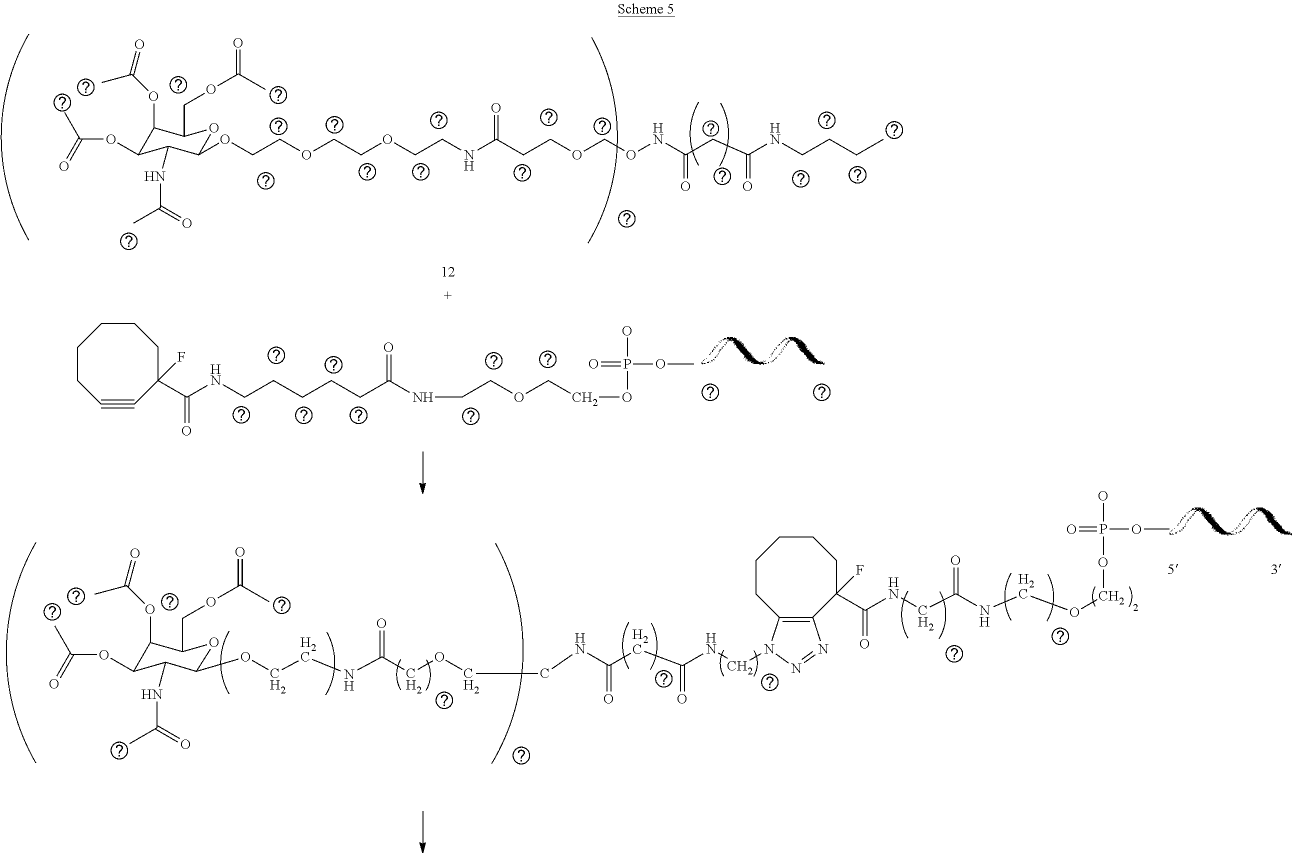

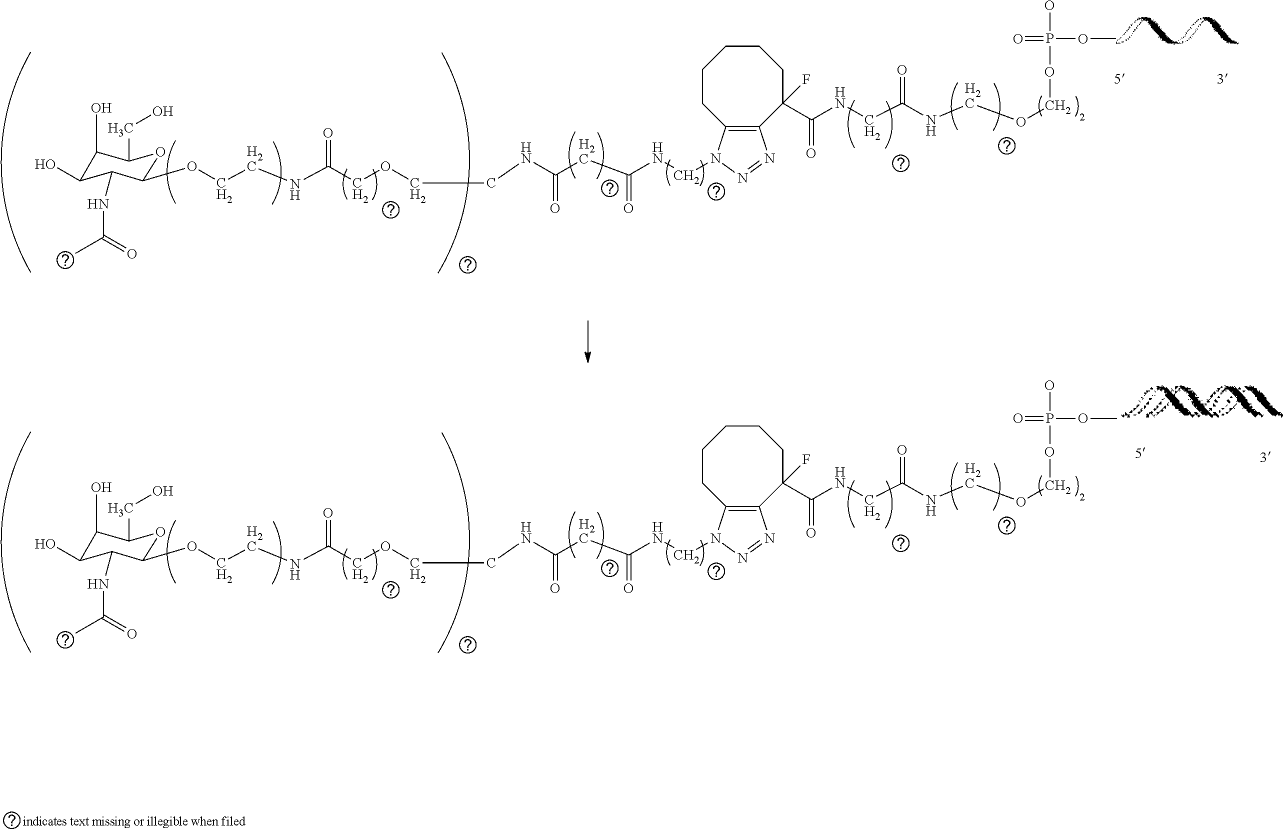

The following constructs are used in examples 3 and 4:

TABLE 12

| Target | ID | Sense Sequence 5′ → 3′ | Antisense Sequence 5′ → 3′ |
|---|---|---|---|
| hsHA01 | ETX006 | (invabasic)(invabasic)gsascuuuCfaUfC/ | usAfsuauUfuCfCfaggaUfgAfaaguescsa |
| | | Cfuggaaauasusa(NHC6)(ET-GaINAc-T2CO) | |
| hsHA01 | ETX002 | (ET-GalINAc-T2CO)(NH2C12)gacunuCfaUfC | usAfsuanUfuCfCfaggaUfgAfaagucscsa |
| | | fCfuggaaauasusa(invabasic)(invabasic) | |
| hsC5 | ETX011 | (ET-GalNAc-T2CO)(NH2C12)aaGfcAfaGfaUfA | usAfsUfuAfuaAfaAfauaUfcUfuGfcuusu |
| | | fUfuUfuuAfuAfasusa(invabasic)(invabasic) | sudTdT |
| hsC5 | ETX015 | (invabasic)(invabasic)asasGfcAfaGfaUfA | usAfsUfuAfuaAfaAfauaUfcUfuGfcuusu |
| | | fUfuUfuuAfuAfaua(NHC6)(ET-GalNAc-T2C0) | sudTdT |
| hsTTR | ETX020 | ET-GalNAc-T2CO)(NH2C12)uggganUfuCfA | usCfsuugGfuuAfcaugAfaAfucccasusc |
| | | fUfguaaccaasgsa(invabasic)(invabasic) | |
| hsTTR | ETX024 | (invabasic)(invabasic)usgsggauUfuCfAfU | usCfsuugGfuuAfcaugAfaAfucccasusc |
| | | fguaaccaaga(NHC6)(ET-GalNAc-T2C0) | |

In Table 12 the components in brackets having the following nomenclature (NHC6), (NH2C12) and (ET-GalNAc-T2CO) are descriptors of elements of the linkers, and the complete corresponding linker structures are shown in FIG. 32 and FIG. 33 herein. This correspondence of abbreviation to actual linker structure similarly applies to all other references of the above abbreviations herein.

TABLE 12a

| | | | Linker plus ligand |
|---|---|---|---|
| Target | ID | Short Descriptor | SiRNA as Table 12 |
| hsHAO1 | ETX006 | 3′-GalNAc T2a | Linker + ligand as FIG. 32 |
| | | inverted abasic | |
| hsHAO1 | ETX002 | 5′-GalNAc T2b | Linker + ligand as FIG. 33 |
| | | inverted abasic | |
| hsC5 | ETX011 | 5′-GalNAc T2b | Linker + ligand as FIG. 33 |
| | | inverted abasic | |
| hsC5 | ETX015 | 3′-GalNAc T2a | Linker + ligand as FIG. 32 |
| | | inverted abasic | |
| hsTTR | ETX020 | 5′-GalNAc T2b | Linker + ligand as FIG. 33 |
| | | inverted abasic | |
| hsTTR | ETX024 | 3′-GalNAc T2a | Linker + ligand as FIG. 32 |
| | | inverted abasic | |

The following control constructs are also used in the examples:

TABLE 13

| Target | ID | Sense Sequence 5′ → 3′ | Antisense Sequence 5′ → 3′ |
|---|---|---|---|
| F-Luc | XD- | cuuAcGcuGAGuAcuucGAdTsdT | UCGAAGUACUCAGCGuAAGdTsdT |
| | 00914 | | |
| hsFVII | XD- | AGAuAuGcAcAcAcAcGGAdTsdT | UCCGUGUGUGUGcAuAUCUdTsdT |
| | 03999 | | |
| hsAHSA1 | XD- | uscsUfcGfuGfgCfcUfuAfaUf | UfsUfsuCfaUfuAfaGfgCfcAf |
| | 15421 | gAfaAf(invdT) | cGfaGfasusu |

### Abbreviations

AHSA1 Activator of heat shock protein ATPase1
ASGR1 Asialoglycoprotein Receptor 1
ASO Antisense oligonucleoside
bDNA branched DNA
bp base-pair
C5 complement C5
conc. concentration
ctrl. control
CV coefficient of variation
dG, dC, dA, dT DNA residues
F Fluoro
FCS fetal calf serum
GalNAc N-Acetylgalactosamine
GAPDH Glyceraldehyde 3-phosphate dehydrogenase
G, C, A, U RNA residues
g, c, a, u 2′-O-Methyl modified residues
Gf, Cf, Af, Uf 2′-Fluoro modified residues
h hour
HAO1 Hydroxyacid Oxidase 1
HPLC High performance liquid chromatography
Hs Homo sapiens
IC50 concentration of an inhibitor where the response is reduced by 50%
ID identifier
KD knockdown
LF2000 Lipofectamine2000
M molar
Mf Macaca fascicularis
min minute
MV mean value
n.a. or N/A not applicable
NEAA non-essential amino acid
nt nucleoside
QC Quality control
QG2.0 QuantiGene 2.0
RLU relative light unit
RNAi RNA interference
RT room temperature
s Phosphorothioate backbone modification
SAR structure-activity relationship
SD standard deviation
siRNA small interfering RNA
TTR Transthyretin

### Example 3

### Summary

GalNAc-siRNAs targeting either hsHAO1, hsC5 or hsTTR mRNA were synthesized and QC-ed. The entire set of siRNAs (except siRNAs targeting HAO1) was first studied in a dose-response setup in HepG2 cells by transfection using RNAiMAX, followed by a dose-response analysis in a gymnotic free uptake setup in primary human hepatocytes.

Direct incubation of primary human hepatocytes with GalNAc-siRNAs targeting hsHAO1, hsC5 or hsTTR mRNA resulted in dose-dependent on-target mRNA silencing to varying degrees.

### Aim of Study

The aim of this set of experiments was to analyze the in vitro activity of different GalNAc-ligands in the context of siRNAs targeting three different on-targets, namely hsHAO1, hsC5 or hsTTR mRNA.

Work packages of this study included (i) assay development to design, synthesize and test bDNA probe sets specific for each and every individual on-target of interest, (ii) to identify a cell line suitable for subsequent screening experiments, (iii) dose-response analysis of potentially all siRNAs (by transfection) in one or more human cancer cell lines, and (iv) dose-response analysis of siRNAs in primary human hepatocytes in a gymnotic, free uptake setting. In both settings, IC50 values and maximal inhibition values should be calculated followed by ranking of the siRNA study set according to their potency.

### Material and Methods

### Oligonucleoside Synthesis

Standard solid-phase synthesis methods were used to chemically synthesize siRNAs of interest (see Table 12) as well as controls (see Table 13).

### Cell Culture and In-Vitro Transfection Experiments

Cell culture, transfection and QuantiGene2.0 branched DNA assay are described below, and siRNA sequences are listed in Tables 12 and 13. HepG2 cells were supplied by American Tissue Culture Collection (ATCC) (HB-8065, Lot #: 63176294) and cultured in ATCC-formulated Eagle's Minimum Essential Medium supplemented to contain 10% fetal calf serum (FCS). Primary human hepatocytes (PHHs) were sourced from Primacyt (Schwerin, Germany) (Lot #: CyHuf19009HEc). Cells are derived from a malignant glioblastoma tumor by explant technique. All cells used in this study were cultured at 37° C. in an atmosphere with 5% CO₂ in a humidified incubator.

For transfection of HepG2 cells with hsC5 or hsTTR targeting siRNAs (and controls), cells were seeded at a density of 20.000 cells/well in regular 96-well tissue culture plates. Transfection of cells with siRNAs was carried out using the commercially available transfection reagent RNAiMAX (Invitrogen/Life Technologies) according to the manufacturer's instructions. 10 point dose-response experiments of 20 candidates (11×hsC5, 9×hsTTR) were done in HepG2 cells with final siRNA concentrations of 24, 6, 1.5, 0.4, 0.1, 0.03, 0.008, 0.002, 0.0005 and 0.0001 nM, respectively.

Dose response analysis in PHHs was done by direct incubation of cells in a gymnotic, free uptake setting starting with 1.5 μM highest final siRNA concentration, followed by 500 nM and from there on going serially down in twofold dilution steps.

Control wells were transfected into HepG2 cells or directly incubated with primary human hepatocytes at the highest test siRNA concentrations studied on the corresponding plate. All control siRNAs included in the different project phases next to mock treatment of cells are summarized and listed in Table 13. For each siRNA and control, at least four wells were transfected/directly incubated in parallel, and individual data points were collected from each well.

After 24 h of incubation with siRNA post-transfection, media was removed and HepG2 cells were lysed in Lysis Mixture (1 volume of lysis buffer plus 2 volumes of nuclease-free water) and then incubated at 53° C. for at least 45 minutes. In the case of PHHs, plating media was removed 5 h post treatment of cells followed by addition of 50 μl of complete maintenance medium per well. Media was exchanged in that way every 24 h up to a total incubation period of 72 h. At either 4 h or 72 h time point, cell culture supernatant was removed followed by addition of 200 μl of Lysis Mixture supplemented with 1:1000 v/v of Proteinase K.

The branched DNA (bDNA) assay was performed according to manufacturer's instructions. Luminescence was read using a 1420 Luminescence Counter (WALLAC VICTOR Light, Perkin Elmer, Rodgau-Jügesheim, Germany) following 30 minutes incubation in the presence of substrate in the dark. For each well, the on-target mRNA levels were normalized to the hsGAPDH mRNA levels. The activity of any siRNA was expressed as percent on-target mRNA concentration (normalized to hsGAPDH mRNA) in treated cells, relative to the mean on-target mRNA concentration (normalized to hsGAPDH mRNA) across control wells.

### Assay Development

QuantiGene2.0 branched DNA (bDNA) probe sets were designed and synthesised specific for Homo sapiens GAPDH, AHSA1, hsHAO1, hsC5 and hsTTR. bDNA probe sets were initially tested by bDNA analysis according to manufacturer's instructions, with evaluation of levels of mRNAs of interest in two different lysate amounts, namely 10 μl and 50 μl, of the following human and monkey cancer cell lines next to primary human hepatocytes: SJSA-1, TF1, NCI-H1650, Y-79, Kasumi-1, EAhy926, Caki-1, Colo205, RPTEC, A253, HeLaS3, Hep3B, BxPC3, DU145, THP-1, NCI-H460, IGR37, LS174T, Be(2)-C, SW 1573, NCI-H358, TC71, 22Rv1, BT474, HeLa, KBwt, Panc-1, U87MG, A172, C42, HepG2, LNCaP, PC3, SupT11, A549, HCT116, HuH7, MCF7, SH-SY5Y, HUVEC, C33A, HEK293, HT29, MOLM 13 and SK-MEL-2. Wells containing only bDNA probe set without the addition of cell lysate were used to monitor technical background and noise signal.

### Results

### Identification of Suitable Cell Types for Screening of GalNAc-siRNAs

FIG. 1 to FIG. 3 show mRNA expression data for the three on-targets of interest, namely hsC5, hsHAO1 and hsTTR, in lysates of a diverse set of human cancer cell lines plus primary human hepatocytes. Cell numbers per lysate volume are identical with each cell line tested, this is necessary to allow comparisons of expression levels amongst different cell types. FIG. 1 shows hsC5 mRNA expression data for all cell types tested.

The identical type of cells were also screened for expression of hsHAO1 mRNA, results are shown in bar diagrams as part of FIG. 2.

Lastly, suitable cell types were identified which would allow for screening of GalNAc-siRNAs targeting hsTTR, respective data are part of FIG. 3.

In summary, mRNA expression levels for all three on-targets of interest are high enough in primary human hepatocytes (PHHs). Further, HepG2 cells could be used to screen GalNAc-siRNAs targeting hsC5 and hsTTR mRNAs, in contrast, no cancer cell line could be identified which would be suitable to test siRNAs specific for hsHAO1 mRNA.

Dose-Response Analysis of hsTTR Targeting GalNAc-siRNAs in HepG2 Cells

Following transfection optimization, HepG2 cells were transfected with the entire set of hsTTR targeting GalNAc-siRNAs (see Table 12) in a dose-response setup using RNAiMAX. The highest final siRNA test concentration was 24 nM, going down in ninecells. Table 14 lists activity data for all hsTTR targeting GalNAC-siRNAs studied.

TABLE 14
Target, incubation time, external ID, IC20/IC50/IC80
values and maximal inhibition
of hsTTR targeting siRNAs in HepG2 cells. The
listing is ordered according to external ID, with 4 h of
incubation listed on top and 24 h of incubation on the bottom.

| | Incu- | | | | | Max. |
|---|---|---|---|---|---|---|
| | bation | External | IC20 | IC50 | IC80 | Inhib. |
| Target | [h] | ID | [nM] | [nM] | [nM] | [%] |
| hsTTR | 4 | ETX020 | 1.953 | #N/A | #N/A | 37.9 |
| hsTTR | 4 | ETX024 | 1.952 | #N/A | #N/A | 48.2 |
| hsTTR | 24 | ETX020 | 0.005 | 0.025 | 0.133 | 95.5 |
| hsTTR | 24 | ETX024 | 0.008 | 0.029 | 0.134 | 95.5 |

Results for the 24 h incubation are also shown in FIGS. 11A-B

In general, transfection of HepG2 cells with hsTTR targeting siRNAs results in on-target mRNA silencing spanning in general the entire activity range from 0% silencing to maximal inhibition. Data generated 24 h post transfection are more robust with lower standard variations, as compared to data generated only 4 h post transfection. Further, the extent of on-target knockdown generally increases over time from 4 h up to 24 h of incubation. hsTTR GalNAc-siRNAs have been identified that silence the on-target mRNA>95% with IC50 values in the low double-digit pM range.

Dose-Response Analysis of hsC5 Targeting GalNAc-siRNAs in HepG2 Cells

The second target of interest, hsC5 mRNA, was tested in an identical dose-response setup (with minimally different final siRNA test concentrations, however) by transfection of HepG2 cells using RNAiMAX with GalNAc-siRNAs sharing identical linger/position/GalNAc-ligand variations as with hsTTR siRNAs, but sequences specific for the on-target hsC5 mRNA.

TABLE 15
Target, incubation time, external ID, IC20/IC50/IC80 values and
maximal inhibition of hsC5 targeting siRNAs in HepG2 cells.
The listing is ordered according to external ID, with 4 h of
incubation listed on top and 24 h of incubation on the bottom.

| | Incu- | | | | | Max. |
|---|---|---|---|---|---|---|
| | bation | External | IC20 | IC50 | IC80 | Inhib. |
| Target | [h] | ID | [nM] | [nM] | [nM] | [%] |
| C5 | 4 | ETX011 | 0.091 | 0.424 | #N/A | 74.6 |
| C5 | 4 | ETX015 | 0.407 | 0.578 | #N/A | 61.9 |
| C5 | 24 | ETX011 | 0.001 | 0.005 | 0.045 | 88.4 |
| C5 | 24 | ETX015 | 0.003 | 0.013 | 0.099 | 88.8 |

Results for the 24 h incubation are also shown in FIGS. 12A-B

There is dose-dependent on-target hsC5 mRNA silencing upon transfection of HepG2 cells with the GalNAc-siRNA set specific for hsC5. Some knockdown can already be detected at 4 h post-transfection of cells, an even higher on-target silencing is observed after a longer incubation period, namely 24 h. hsC5 GalNAc-siRNAs have been identified that silence the on-target mRNA almost 90% with IC50 values in the low single-digit pM range.

### Identification of a Primary Human Hepatocyte Batch Suitable for Testing of all GalNAc-siRNAs

The dose-response analysis of the two GalNAc-siRNA sets in human cancer cell line HepG2 should demonstrate (and ensure) that all new GalNAc-/linker/position/cap variants are indeed substrates for efficient binding to AGO2 and loading into RISC, and in addition, able to function in RNAi-mediated cleavage of target mRNA. However, in order to test whether the targeting GalNAc-ligand derivatives allow for efficient uptake into hepatocytes, dose-response analysis experiments should be done in primary human hepatocytes by gymnotic, free uptake setup. Hepatocytes do exclusively express the Asialoglycoprotein receptor (ASGR1) to high levels, and this receptor generally is used by the liver to remove target glycoproteins from circulation. It is common knowledge by now, that certain types of oligonucleosides, e.g. siRNAs or ASOs, conjugated to GalNAc-ligands are recognized by this high turnover receptor and efficiently taken up into the cytoplasm via clathrin-coated vesicles and trafficking to endosomal compartments. Endosomal escape is thought to be the rate-limiting step for oligonucleoside delivery.

An intermediate assay development experiment was done in which different batches of primary human hepatocytes were tested for their expression levels of relevant genes of interest, namely hsC5, hsTTR, hsHAO1, hsGAPDH and hsAHSA1. Primacyt (Schwerin, Germany) provided three vials of different primary human hepatocyte batches for testing, namely BHuf16087, CHF2101 and CyHuf19009. The cells were seeded on collagen-coated 96-well tissue culture plates, followed by incubation of cells for 0 h, 24 h, 48 h and 72 h before cell lysis and bDNA analysis to monitor mRNA levels of interest. FIG. 6 shows the absolute mRNA expression data for all three on-targets of interest—hsTTR, hsC5 and hsHAO1—in the primary human hepatocyte batches BHuf16087, CHF2101 and CyHuf19009. mRNA expression levels of hsGAPDH and hsAHSA1 are shown in FIG. 7.

Overall, the mRNA expression of all three on-targets of interest in the primary human hepatocyte batches BHuf16087 and CyHuf19009 are high enough after 72 h to continue with the bDNA assay. Due to the total amount of vials available for further experiments, we continued the experiments with the batch CyHuf19009.

Dose-Response Analysis of hsHAO1 Targeting GalNAc-siRNAs in PHHs

Following the identification of a suitable batch (CyHuf19009) of primary human hepatocytes (PHHs), a gymnotic, free uptake analysis was performed of hsHAO1 targeting GalNAc-siRNAs, listed in Table 12. The highest tested final siRNA concentration was 1.5 μM, followed by 500 nM, going down in eight two-fold serial dilution steps to the lowest final siRNA concentration of 1.95 nM. The experiments ended at 4 h and 72 h post direct incubation of PHH cells. Table 16 lists activity data for all hsHAO1 targeting GalNAc-siRNAs studied. All control siRNAs included in this experiment are summarized and listed in Table 13.

TABLE 16
Target, incubation time, external ID, IC20/IC50/IC80 values and
maximal inhibition of hsHAO1 targeting GalNAc-siRNAs in
primary human hepatocytes (PHHs). The listing is
organized according to external ID, with 4 h and 72 h
incubation listed on top and bottom, respectively.

| | Incu- | | | | | Max. |
|---|---|---|---|---|---|---|
| | bation | External | IC20 | IC50 | IC80 | Inhib. |
| Target | [h] | ID | [nM] | [nM] | [nM] | [%] |
| hsHAO1 | 4 | ETX002 | #N/A | #N/A | #N/A | 7.2 |
| (hsGO1) | | | | | | |
| hsHAO1 | 4 | ETX006 | #N/A | #N/A | #N/A | 0.5 |
| (hsGO1) | | | | | | |
| hsHAO1 | 72 | ETX002 | 23.9 | #N/A | #N/A | 44.3 |
| (hsGO1) | | | | | | |
| hsHAO1 | 72 | ETX006 | 27.5 | 617.1 | #N/A | 53.6 |
| (hsGO1) | | | | | | |

Results for the 72 h incubation are also shown in FIGS. 13A-B.

Gymnotic, free uptake of GalNAc-siRNAs targeting hsHAO1 did not lead to significant on-target silencing within 4 h, however after 72 h incubation on-target silencing was visible in a range of 35.5 to 58.1% maximal inhibition.

Dose-Response Analysis of hsC5 Targeting GalNAc-siRNAs in PHHs

The second target of interest, hsC5 miRNA, was tested in an identical dose-response setup by gymnotic, free uptake in PHHs with GalNAc-siRNAs sharing identical linker/position/GalNAC-ligand variations as with hsTTR and hsHAO1 tested in the assays before, but sequences specific for the on-target hsC5 mRNA. Sequences for the GalNAc-siRNAs targeting hsC5 and all sequences and information about control siRNAs are listed in Table 12 and Table 13, respectively. The experiment ended after 4 h and 72 h direct incubation of PHHs. Table 17 lists activity data for all hsC5 targeting GalNAc-siRNAs studied.

TABLE 17
Target, incubation time, external ID, IC20/IC50/IC80 values and
maximal inhibition of hsC5 targeting GalNAc-siRNAs in PHHs.
The listing is organized according to external ID, with 4 h
and 72 h incubation listed on top and bottom, respectively.

| | Incu- | | | | | Max. |
|---|---|---|---|---|---|---|
| | bation | External | IC20 | IC50 | IC80 | Inhib. |
| Target | [h] | ID | [nM] | [nM] | [nM] | [%] |
| C5 | 4 | ETX011 | #N/A | #N/A | #N/A | −2.9 |
| C5 | 4 | ETX015 | #N/A | #N/A | #N/A | 7.6 |
| C5 | 72 | ETX011 | 2.6 | 295.3 | #N/A | 62.1 |
| C5 | 72 | ETX015 | 7.2 | 315.0 | #N/A | 57.2 |

Results for the 72 h incubation are also shown in FIGS. 14A-B.

No significant on-target silencing of GalNAc-siRNAs is visible after 4 h incubation. Data generated after an incubation period of 72 h showed a more robust on-target silencing of up to 65.5% maximal inhibition.

Dose-Response Analysis of hsTTR Targeting GalNAc-siRNAs in PHHs

The last target of interest, hsTTR mRNA, was again tested in a gymnotic, free uptake in PHHs in an identical dose-response setup as for the targets hsHAO1 and hsC5, with the only difference being that specific siRNA sequences for the on-target hsTTR mRNA was used (see Table 12).

The experiment ended after 72 h of direct incubation of PHHs. Table 18 lists activity data for all hsTTR targeting GalNAc-siRNAs studied.

TABLE 18
Target, incubation time, external ID, IC20/IC50/IC80 values
and maximal inhibition of hsTTR targeting GalNAc-siRNAs
in primary human hepatocytes (PHHs).
The listing is organized according to external ID.

| | Incu- | | | | | Max. |
|---|---|---|---|---|---|---|
| | bation | External | IC20 | IC50 | IC80 | Inhib. |
| Target | [h] | ID | [nM] | [nM] | [nM] | [%] |
| hsTTR | 72 | ETX020 | 2.2 | 31.0 | #N/A | 78.4 |
| hsTTR | 72 | ETX024 | 9.5 | 110.0 | #N/A | 71.3 |

Results are also shown in FIGS. 15A-B.

Gymnotic, free uptake of GalNAc-siRNAs targeting hsTTR did lead to significant on-target silencing within 72 h, ranging between 46 to 82.5% maximal inhibition.

### Conclusions and Discussion

The scope of this study was to analyze the in vitro activity of GalNAc-ligands according to the present invention when used in the context of siRNAs targeting three different on-targets, namely hsHAO1, hsC5 and hsTTR mRNA. siRNA sets specific for each target were composed of siRNAs with different linker/cap/modification/GalNAc-ligand chemistries in the context of two different antisense strands each.

For all targets, GalNAc-siRNAs from Table 12 were identified that showed a high overall potency and low IC50 value.

### Example 4

### Routes of Synthesis

vii) Synthesis of the Conjugate Building Blocks TriGalNAc

Thin layer chromatography (TLC) was performed on silica-coated aluminium plates with fluorescence indicator 254 nm from Macherey-Nagel. Compounds were visualized under UV light (254 nm), or after spraying with the 5% H₂SO₄ in methanol (MeOH) or ninhydrin reagent according to Stahl (from Sigma-Aldrich), followed by heating. Flash chromatography was performed with a Biotage Isolera One flash chromatography instrument equipped with a dual variable UV wavelength detector (200-400 nm) using Biotage Sfar Silica 10, 25, 50 or 100 g columns (Uppsala, Sweden).

All moisture-sensitive reactions were carried out under anhydrous conditions using dry glassware, anhydrous solvents and argon atmosphere. All commercially available reagents were purchased from Sigma-Aldrich and solvents from Carl Roth GmbH+Co. KG. D-Galactosamine pentaacetate was purchased from AK scientific.

HPLC/ESI-MS was performed on a Dionex UltiMate 3000 RS UHPLC system and Thermo Scientific MSQ Plus Mass spectrometer using an Acquity UPLC Protein BEH C4 column from Waters (300 Å, 1.7 μm, 2.1×100 mm) at 60° C. The solvent system consisted of solvent A with H₂O containing 0.1% formic acid and solvent B with acetonitrile (ACN) containing 0.1% formic acid. A gradient from 5-100% of B over 15 min with a flow rate of 0.4 mL/min was employed. Detector and conditions: Corona ultra-charged aerosol detection (from esa). Nebulizer Temp.: 25° C. N₂ pressure: 35.1 psi. Filter: Corona.

¹H and ¹³C NMR spectra were recorded at room temperature on a Varian spectrometer at 500 MHz (¹H NMR) and 125 MHz (¹³C NMR). Chemical shifts are given in ppm referenced to the solvent residual peak (CDCl₃—1H NMR: δ at 7.26 ppm and ¹³C NMR δ at 77.2 ppm; DMSO-d₆-¹H NMR: δ at 2.50 ppm and ¹³C NMR δ at 39.5 ppm). Coupling constants are given in Hertz. Signal splitting patterns are described as singlet (s), doublet (d), triplet (t) or multiplet (m).

viii) Synthesis Route for the Conjugate Building Block TriGalNAc

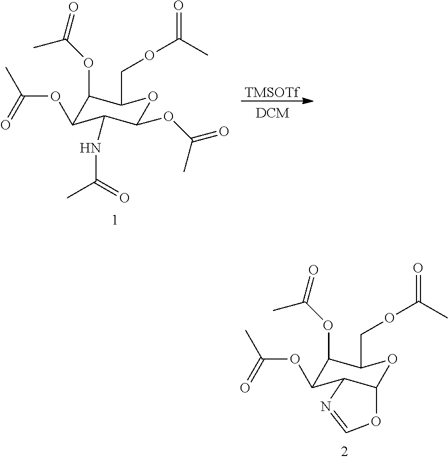 [SMILES: "TMSOTf" ; CC(=O)NC1C(OC(C)=O)OC(COC(C)=O)C(OC(C)=O)C1OC(C)=O ; CC(=O)OCC1OC2OC=NC2C(OC(C)=O)C1OC(C)=O]

Preparation of compound 2: D-Galactosamine pentaacetate (3.00 g, 7.71 mmol, 1.0 eq.) was dissolved in anhydrous dichloromethane (DCM) (30 ml) under argon and trimethylsilyl trifluoromethanesulfonate (TMSOTf, 4.28 g, 19.27 mmol, 2.5 eq.) was added. The reaction was stirred at room temperature for 3 h. The reaction mixture was diluted with DCM (50 mL) and washed with cold saturated aq. NaHCO₃ (100 mL) and water (100 mL). The organic layer was separated, dried over Na2SO4 and concentrated to afford the title compound as yellow oil, which was purified by flash chromatography (gradient elution: 0-10% MeOH in DCM in 10 CV). The product was obtained as colourless oil (2.5 g, 98%, rf=0.45 (2% MeOH in DCM)).

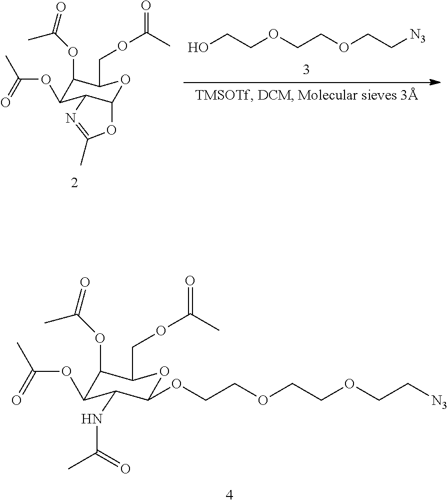

Preparation of compound 4: Compound 2 (2.30 g, 6.98 mmol, 1.0 eq.) and azido-PEG3-OH (1.83 g, 10.5 mmol, 1.5 eq.) were dissolved in anhydrous DCM (40 mL) under argon and molecular sieves 3 Å (5 g) was added to the solution. The mixture was stirred at room temperature for 1 h. TMSOTf (0.77 g, 3.49 mmol, 0.5 eq.) was then added to the mixture and the reaction was stirred overnight. The molecular sieves were filtered, the filtrate was diluted with DCM (100 mL) and washed with cold saturated aq. NaHCO₃ (100 mL) and water (100 mL). The organic layer was separated, dried over Na₂SO₄ and the solvent was removed under reduced pressure. The crude material was purified by flash chromatography (gradient elution: 0-3% MeOH in DCM in 10 CV) to afford the title product as light yellow oil (3.10 g, 88%, rf=0.25 (2% MeOH in DCM)). MS: calculated for C₂₀H₃₂N₄O₁₁, 504.21. Found 505.4. ¹H NMR (500 MHz, CDCl₃) δ 6.21-6.14 (m, 1H), 5.30 (dd, J=3.4, 1.1 Hz, 1H), 5.04 (dd, J=11.2, 3.4 Hz, 1H), 4.76 (d, J=8.6 Hz, 1H), 4.23-4.08 (m, 3H), 3.91-3.80 (m, 3H), 3.74-3.59 (m, 9H), 3.49-3.41 (m, 2H), 2.14 (s, 3H), 2.02 (s, 3H), 1.97 (d, J=4.2 Hz, 6H). ¹³C NMR (125 MHz, CDCl₃) δ 170.6 (C), 170.5 (C), 170.4 (C), 170.3 (C), 102.1 (CH), 71.6 (CH), 70.8 (CH), 70.6 (CH), 70.5 (CH), 70.3 (CH₂), 69.7 (CH₂), 68.5 (CH₂), 66.6 (CH₂), 61.5 (CH₂), 23.1 (CH₃), 20.7 (3×CH₃).

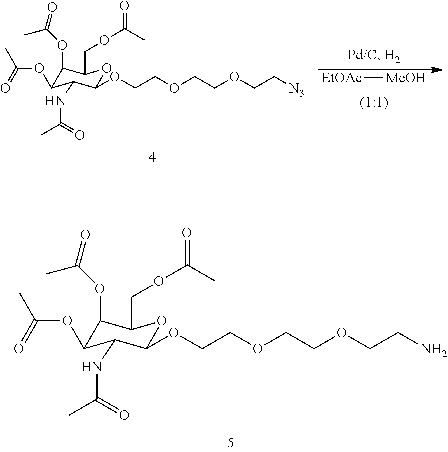

Preparation of compound 5: Compound 4 (1.00 g, 1.98 mmol, 1.0 eq.) was dissolved in a mixture of ethyl acetate (EtOAc) and MeOH (30 mL 1:1 v/v) and Pd/C (100 mg) was added. The reaction mixture was degassed using vacuum/argon cycles (3×) and hydrogenated under balloon pressure overnight. The reaction mixture was filtered through celite and washed with EtOAc (30 mL). The solvent was removed under reduced pressure to afford the title compound as colourless oil (0.95 g, quantitative yield, rf=0.25 (10% MeOH in DCM)). The compound was used without further purification. MS: calculated for C₂₀H₃₄N₂O₁₁, 478.2. Found 479.4.

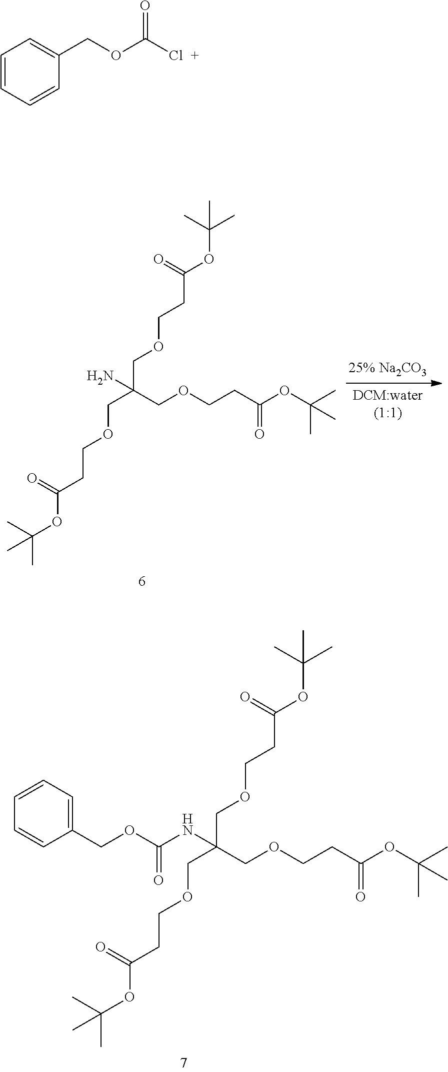 [SMILES: O=C(Cl)OCc1ccccc1 ; CC(C)(C)OC(=O)CCOCC(N)(COCCC(=O)OC(C)(C)C)COCCC(=O)OC(C)(C)C ; CC(C)(C)OC(=O)CCOCC(COCCC(=O)OC(C)(C)C)(COCCC(=O)OC(C)(C)C)NC(=O)OCc1ccccc1]

Preparation of compound 7: Tris{[2-(tert-butoxycarbonyl)ethoxy]methyl}-methylamine 6 (3.37 g, 6.67 mmol, 1.0 eq.) was dissolved in a mixture of DCM/water (40 mL 1:1 v/v) and Na₂CO₃ (0.18 g, 1.7 mmol, 0.25 eq.) was added while stirring vigorously. Benzyl chloroformate (2.94 mL, 20.7 mmol, 3.10 eq.) was added dropwise to the previous mixture and the reaction was stirred at room temperature for 24 h. The reaction mixture was diluted with CH₂Cl₂ (100 mL) and washed with water (100 mL). The organic layer was separated and dried over Na₂SO₄. The solvent was removed under reduced pressure and the resulting crude material was purified by flash chromatography (gradient elution: 0-10% EtOAc in cyclohexane in 12 CV) to afford the title compound as pale yellowish oil (3.9 g, 91%, rf=0.56 (10% EtOAc in cyclohexane)). MS: calculated for C₃₃H₅₃NO₁₁, 639.3. Found 640.9. ¹H NMR (500 MHz, DMSO-d₆) δ 7.38-7.26 (m, 5H), 4.97 (s, 2H), 3.54 (t, 6H), 3.50 (s, 6H), 2.38 (t, 6H), 1.39 (s, 27H). ¹³C NMR (125 MHz, DMSO-d₆) δ 170.3 (3×C), 154.5 (C), 137.1 (C), 128.2 (2×CH), 127.7 (CH), 127.6 (2×CH), 79.7 (3×C), 68.4 (3×CH₂), 66.8 (3×CH₂), 64.9 (C), 58.7 (CH₂), 35.8 (3×CH₂), 27.7 (9×CH₃).

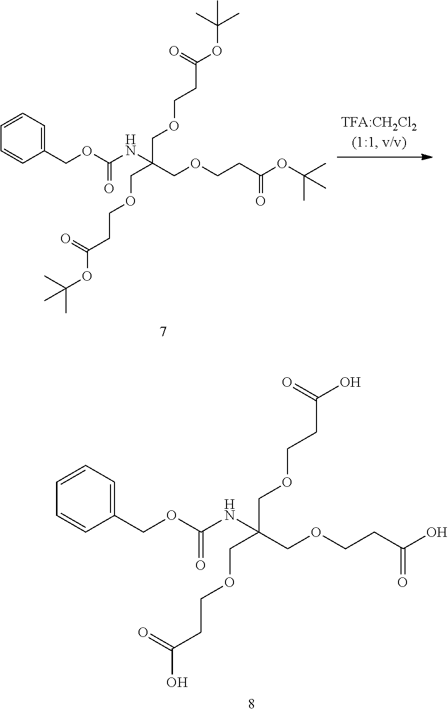 [SMILES: CC(C)(C)OC(=O)CCOCC(COCCC(=O)OC(C)(C)C)(COCCC(=O)OC(C)(C)C)NC(=O)OCc1ccccc1 ; O=C(O)CCOCC(COCCC(=O)O)(COCCC(=O)O)NC(=O)OCc1ccccc1]

Preparation of compound 8: Cbz-NH-tris-Boc-ester 7 (0.20 g, 0.39 mmol, 1.0 eq.) was dissolved in CH₂Cl₂ (1 mL) under argon, trifluoroacetic acid (TFA, 1 ml) was added and the reaction was stirred at room temperature for 1 h. The solvent was removed under reduced pressure, the residue was co-evaporated 3 times with toluene (5 mL) and dried under high vacuum to get the compound as its TFA salt (0.183 g, 98%). The compound was used without further purification. MS: calculated for C₂₁H₂₉NO₁₁, 471.6. Found 472.4.

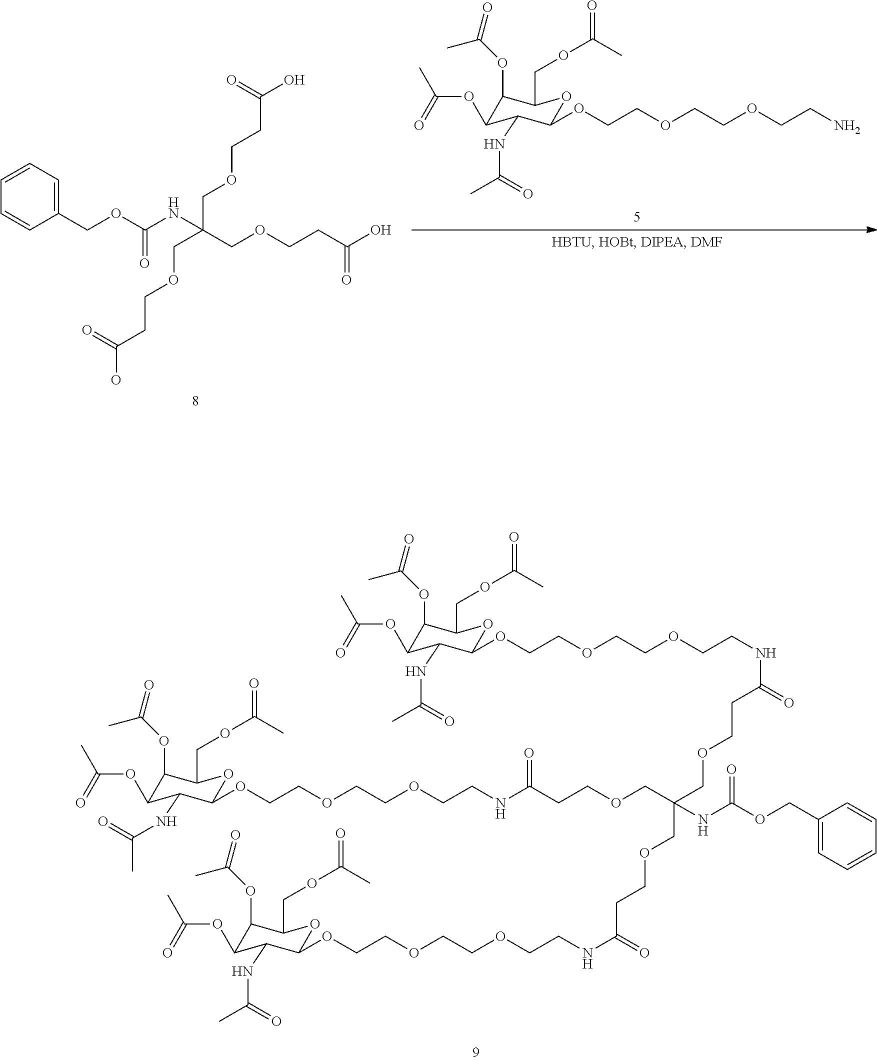 [SMILES: O=C(O)CCOCC(COCCC(=O)O)(COCCC(=O)O)NC(=O)OCc1ccccc1 ; CC(=O)NC1C(OCCOCCOCCN)OC(COC(C)=O)C(OC(C)=O)C1OC(C)=O ; CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)OCc2ccccc2)OC(COC(C)=O)C(OC(C)=O)C1OC(C)=O]

Preparation of compound 9: CbzNH-tris-COOH 8 (0.72 g, 1.49 mmol, 1.0 eq.) and GalNAc-PEG3-NH₂ 5 (3.56 g, 7.44 mmol, 5.0 eq.) were dissolved in N,N-dimethylformamide (DMF) (25 mL). Then N,N,N′,N′-tetramethyl-O-(1H-benzotriazol-1-yl)uronium hexafluorophosphate (HBTU) (2.78 g, 7.44 mmol, 5.0 eq.), 1-hydroxybenzotriazole hydrate (HOBt) (1.05 g, 7.44 mmol, 5.0 eq.) and N,N-diisopropylethylamine (DIPEA) (2.07 mL, 11.9 mmol, 8.0 eq.) were added to the solution and the reaction was stirred for 72 h. The solvent was removed under reduced pressure, the residue was dissolved in DCM (100 ml) and washed with saturated aq. NaHCO₃ (100 mL). The organic layer was dried over Na₂SO₄, the solvent evaporated and the crude material was purified by flash chromatography (gradient elution: 0-5% MeOH in DCM in 14 CV). The product was obtained as pale yellowish oil (1.2 g, 43%, rf=0.20 (5% MeOH in DCM)). MS: calculated for C₈₁H₁₂₅N₇O₄₁, 1852.9. Found 1854.7. ¹H NMR (500 MHz, DMSO-d₆) δ 7.90-7.80 (m, 10H), 7.65-7.62 (m, 4H), 7.47-7.43 (m, 3H), 7.38-7.32 (m, 8H), 5.24-5.22 (m, 3H), 5.02-4.97 (m, 4H), 4.60-4.57 (m, 3H), 4.07-3.90 (m 10H), 3.67-3.36 (m, 70H), 3.23-3.07 (m, 25H), 2.18 (s, 10H), 2.00 (s, 13H), 1.89 (s, 11H), 1.80-1.78 (m, 17H). ¹³C NMR (125 MHz, DMSO-d₆) δ 170.1 (C), 169.8 (C), 169.7 (C), 169.4 (C), 169.2 (C), 169.1 (C), 142.7 (C), 126.3 (CH), 123.9 (CH), 118.7 (CH), 109.7 (CH), 100.8 (CH), 70.5 (CH), 69.8 (CH), 69.6 (CH), 69.5 (CH), 69.3 (CH₂), 69.0 (CH₂), 68.2 (CH₂), 67.2 (CH₂), 66.7 (CH₂), 61.4 (CH₂), 22.6 (CH₂), 22.4 (3×CH₃), 20.7 (9×CH₃).

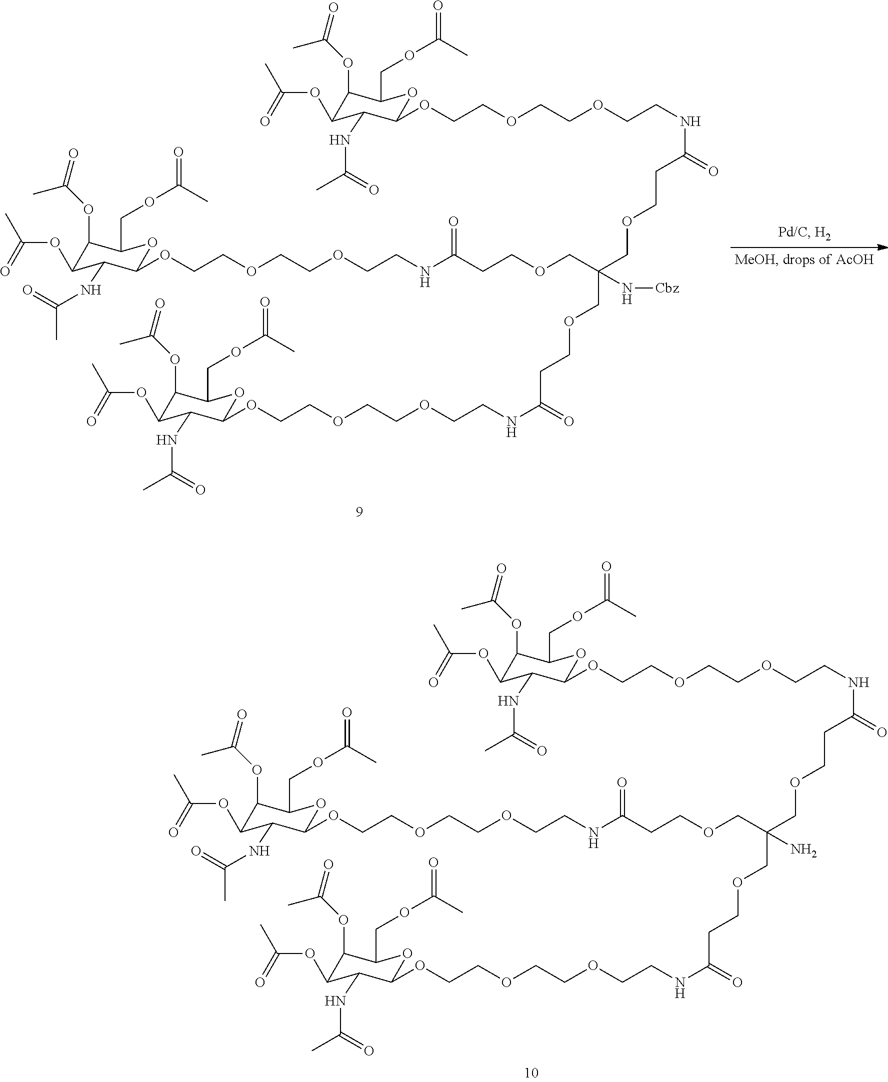 [SMILES: CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)OCc2ccccc2)OC(COC(C)=O)C(OC(C)=O)C1OC(C)=O ; CC(=O)NC1C(OCCOCCOCCNC(=O)CCOCC(N)(COCCC(=O)NCCOCCOCCOC2OC(COC(C)=O)C(OC(C)=O)C(OC(C)=O)C2NC(C)=O)COCCC(=O)NCCOCCOCCOC2OC(COC(C)=O)C(OC(C)=O)C(OC(C)=O)C2NC(C)=O)OC(COC(C)=O)C(OC(C)=O)C1OC(C)=O]

Preparation of compound 10: Triantennary GalNAc compound 9 (0.27 g, 0.14 mmol, 1.0 eq.) was dissolved in MeOH (15 mL), 3 drops of acetic acid (AcOH) and Pd/C (30 mg) was added. The reaction mixture was degassed using vacuum/argon cycles (3×) and hydrogenated under balloon pressure overnight. The completion of the reaction was followed by mass spectrometry and the resulting mixture was filtered through a thin pad of celite. The solvent was evaporated and the residue obtained was dried under high vacuum and used for the next step without further purification. The product was obtained as pale yellowish oil (0.24 g, quantitative yield). MS: calculated for C₇₃H₁₁₉N₇O₃₉, 1718.8. Found 1719.3.

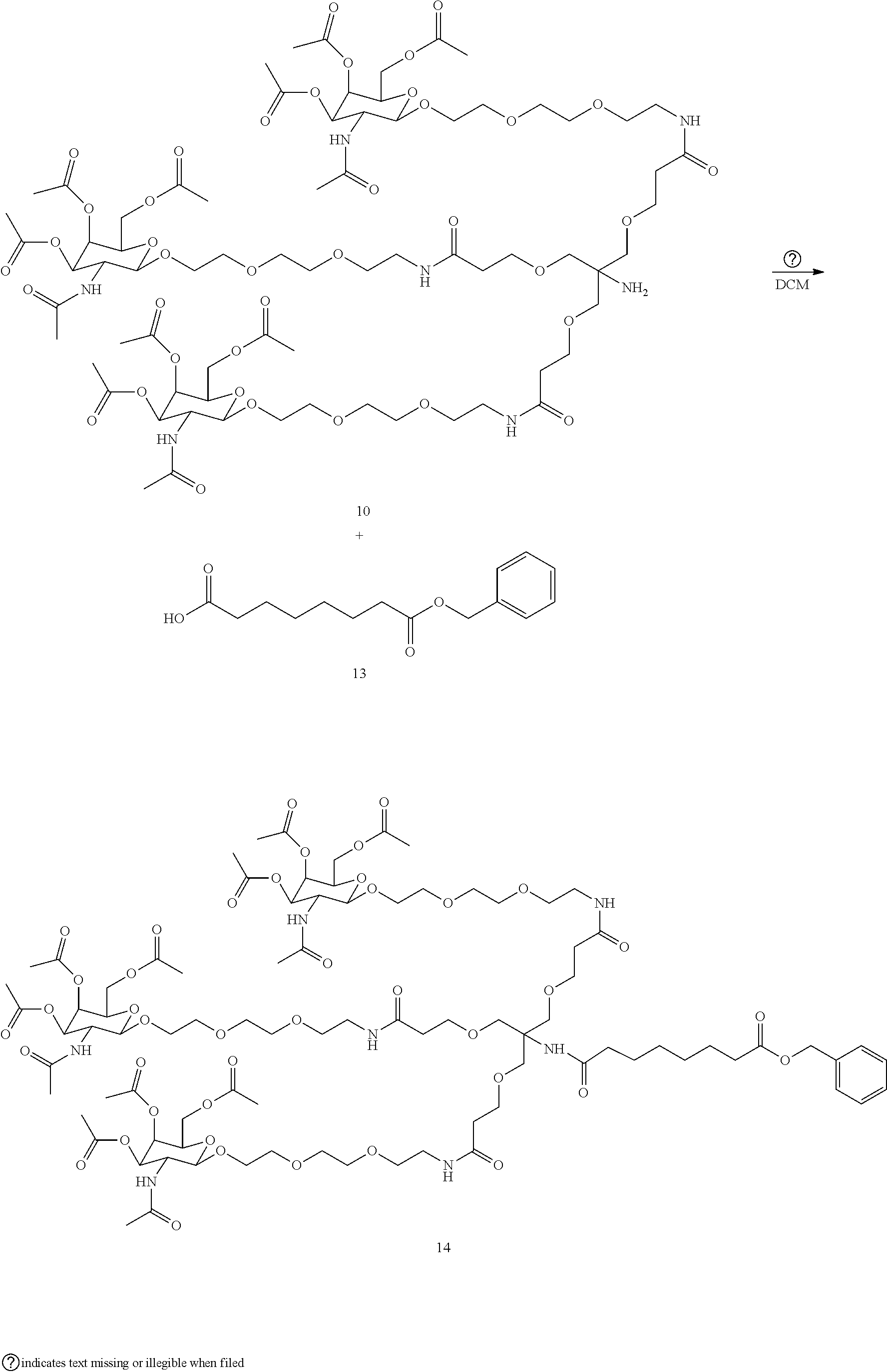 [SMILES: CC(=O)NC1C(OCCOCCOCCNC(=O)CCOCC(N)(COCCC(=O)NCCOCCOCCOC2OC(COC(C)=O)C(OC(C)=O)C(OC(C)=O)C2NC(C)=O)COCCC(=O)NCCOCCOCCOC2OC(COC(C)=O)C(OC(C)=O)C(OC(C)=O)C2NC(C)=O)OC(COC(C)=O)C(OC(C)=O)C1OC(C)=O ; O=C(O)CCCCCCC(=O)OCc1ccccc1 ; CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)CCCCCCC(=O)OCc2ccccc2)OC(COC(C)=O)C(OC(C)=O)C1OC(C)=O]

Preparation of compound 14: Triantennary GalNAc compound 10 (0.45 g, 0.26 mmol, 1.0 eq.), HBTU (0.19 g, 0.53 mmol, 2.0 eq.) and DIPEA (0.23 mL, 1.3 mmol, 5.0 eq.) were dissolved in DCM (10 mL) under argon. To this mixture, it was added dropwise a solution of compound 13 (0.14 g, 0.53 mmol, 2.0 eq.) in DCM (5 mL). The reaction was stirred at room temperature overnight. The solvent was removed and the residue was dissolved in EtOAc (50 mL), washed with water (50 mL) and dried over Na2SO4. The solvent was evaporated and the crude material was purified by flash chromatography (gradient elution: 0-5% MeOH in DCM in 20 CV). The product was obtained as white fluffy solid (0.25 g, 48%, rf=0.4 (10% MeOH in DCM)). MS: calculated for C88H137N7O42, 1965.1. Found 1965.6.

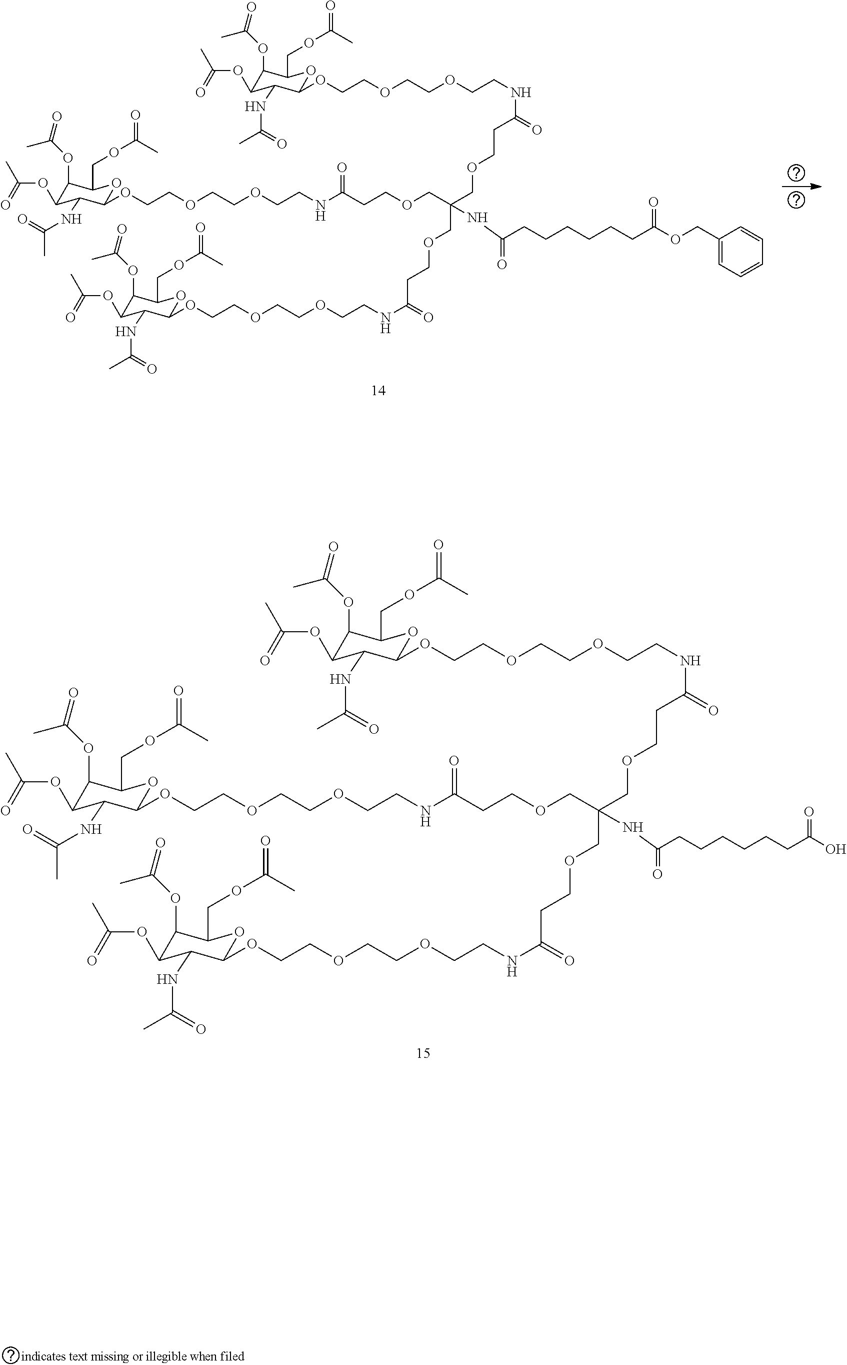 [SMILES: CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)CCCCCCC(=O)OCc2ccccc2)OC(COC(C)=O)C(OC(C)=O)C1OC(C)=O ; CC(=O)NC1C(OCCOCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)CCCCCCC(=O)O)OC(COC(C)=O)C(OC(C)=O)C1OC(C)=O]

Preparation of TriGalNAc (15): Triantennary GalNAc compound 14 (0.31 g, 0.15 mmol, 1.0 eq.) was dissolved in EtOAc (15 mL) and Pd/C (40 mg) was added. The reaction mixture was degassed by using vacuum/argon cycles (3×) and hydrogenated under balloon pressure overnight. The completion of the reaction was monitored by mass spectrometry and the resulting mixture was filtered through a thin pad of celite. The solvent was removed under reduced pressure and the resulting residue was dried under high vacuum over night. The residue was used for conjugations to oligonucleosides without further purification (0.28 g, quantitative yield). MS: calculated for C81H131N7O42, 1874.9. Found 1875.3.

### ix) Oligonucleoside Synthesis

TABLE 19

| Single | | Purity by RP |
|---|---|---|
| strand ID | Sequence 5′ - 3′ | HPLC (%) |
| X91382 | (NH2- | 89.5 |
| | DEG)gacuuuCfaUfCfCfuggaaauasusa(invabasic)(invabasic) | |
| X91383 | (NH2- | 91.6 |
| | DEG)aaGfcAfaGfaUfAfUfuUfuuAfuAfasusa(invabasic)(invabasic) | |
| X91384 | (NH2- | 94.0 |
| | DEG)ugggauUfuCfAfUfguaaccaasgsa(invabasic)(invabasic) | |
| X91403 | (NH2C12)gacuuuCfaUfCfCfuggaaauasusa(invabasic)(invabasic) | 94.2 |
| X91404 | (NH2C12)aaGfcAfaGfaUfAfUfuUfuuAfuAfasusa(invabasic) | 96.5 |
| | (invabasic) | |
| X91405 | (NH2C12)ugggauUfuCfAfUfguaaccaasgsa(invabasic)(invabasic) | 91.3 |
| X91415 | (invabasic)(invabasic)gsascuuuCfaUfCfCfuggaaauasusa(NH2C6) | 96.4 |
| X91416 | (invabasic)(invabasic)asasGfcAfaGfaUfAfUfuUfuuAfuAfaua | 77.4 |
| | (NH2C6) | |
| X91417 | (invabasic)(invabasic)usgsggauUfuCfAfUfguaaccaaga(NH2C6) | 96.7 |
| X91379 | gsascuuuCfaUfCfCfuggaaauaua(GalNAc) | 92.8 |
| X91380 | asasGfcAfaGfaUfAfUfuUfuuAfuAfaua(GalNAc) | 95.7 |
| X91446 | usgsggauUfuCfAfUfguaaccaaga(GalNAc) | 92.1 |
| X38483 | usAfsuauUfuCfCfaggaUfgAfaagucscsa | 91.0 |
| X91381 | usAfsUfuAfuaAfaAfauaUfcUfuGfcuususudTdT | 90.0 |
| X38104 | usCfsuugGfuuAfcaugAfaAfucccasusc | 95.4 |

Af, Cf, Gf, Uf: 2′-F RNA nucleosides
a, c, g, u: 2′-O-Me RNA nucleosides
dT: DNA nucleosides
s: Phosphorothioate
invabasic: 1,2-dideoxyribose
NH2-DEG: Aminoethoxyethyl linker
NH2C12: Aminododecyl linker
NH2C6: Aminohexyl linker

Oligonucleosides were synthesized on solid phase according to the phosphoramidite approach. Depending on the scale either a Mermade 12 (BioAutomation Corporation) or an AKTA Oligopilot (GE Healthcare) was used.

Syntheses were performed on commercially available solid supports made of controlled pore glass either loaded with invabasic (CPG, 480 Å, with a loading of 86 μmol/g; LGC Biosearch cat. #BCG-1047-B) or 2′-F A (CPG, 520 Å, with a loading of 90 μmol/g; LGC Biosearch cat. #BCG-1039-B) or NH2C6 (CPG, 520 Å, with a loading of 85 μmol/g LGC Biosearch cat. #BCG-1397-B) or GalNAc (CPG, 500 Å, with a loading of 57 μmol/g; Primetech) or 2′-O-Methyl C (CPG, 500 Å, with a loading of 84 μmol/g LGC Biosearch cat. #BCG-10-B) or 2′-O-Methyl A (CPG, 497 Å, with a loading of 85 mol/g, LGC Biosearch, Cat. #BCG-1029-B) or dT (CPG, 497 Å, with a loading of 87 μmol/g LGC Biosearch, cat. #BCG-1055-B).

2′-O-Me, 2′-F RNA phosphoramidites and ancillary reagents were purchased from SAFC Proligo (Hamburg, Germany).

2′-O-Methyl phosphoramidites include: 5′-(4,4′-dimethoxytrityl)-N-benzoyl-adenosine 2′-O-methyl-3′-[(2-cyanoethyl)-(N,N-diisopropyl)]-phosphoramidite, 5′-(4,4′-dimethoxytrityl)-N-benzoyl-cytidine 2′-O-methyl-3′-[(2-cyanoethyl)-(N,N-diisopropyl)]-phosphoramidite, 5′-(4,4′-dimethoxytrityl)-N-dimethylformamidine-guanosine 2′-O-methyl-3′-[(2-cyanoethyl)-(N,N-diisopropyl)]-phosphoramidite, 5′-(4,4′-dimethoxytrityl)-uridine 2′-O-methyl-3′-[(2-cyanoethyl)-(N,N-diisopropyl)]-phosphoramidite.

2′-F phosphoramidites include: 5′-dimethoxytrityl-N-benzoyl-deoxyadenosine 2′-fluoro-3′-[(2-cyanoethyl)-(N,N-diisopropyl)]-phosphoramidite, 5′-dimethoxytrityl-N-acetyl-deoxycytidine 2′-fluoro-3′-[(2-cyanoethyl)-(N,N-diisopropyl)]-phosphoramidite, 5′-dimethoxytrityl-N-isobutyryl-deoxyguanosine 2′-fluoro-3′-[(2-cyanoethyl)-(N,N-diisopropyl)]-phosphoramidite and 5′-dimethoxytrityl-deoxyuridine 2′-fluoro-3′-[(2-cyanoethyl)-(N,N-diisopropyl)]-phosphoramidite.

In order to introduce the required amino linkers at the 5′-end of the oligonucleosides the 2-[2-(4-Monomethoxytrityl)aminoethoxy]ethyl-(2-cyanoethyl)-N,N-diisopropyl)-phosphoramidite (Glen Research Cat. #1905) and the 12-(trifluoroacetylamino)dodecyl-[(2-cyanoethyl)-(N,N-diisopropyl)]-phosphoramidite (ChemGenes Cat. #CLP-1575) were employed. The invabasic modification was introduced using 5-O-dimethoxytrityl-1,2-dideoxyribose-3-[(2-cyanoethyl)-(N,N-diisopropyl)]-phosphoramidite (ChemGenes Cat. #ANP-1422).

All building blocks were dissolved in anhydrous acetonitrile (100 mM (Mermade12) or 200 mM (AKTA Oligopilot)) containing molecular sieves (3 Å) except 2′-O-methyl-uridine phosphoramidite which was dissolved in 50% anhydrous DCM in anhydrous acetonitrile. Iodine (50 mM in pyridine/H₂O 9:1 v/v) was used as oxidizing reagent. 5-Ethyl thiotetrazole (ETT, 500 mM in acetonitrile) was used as activator solution. Thiolation for introduction of phosphorthioate linkages was carried out using 100 mM xanthane hydride (TCI, Cat. #6846-35-1) in acetonitrile/pyridine 4:6 v/v.

Coupling times were 5.4 minutes except when stated otherwise. 5′ amino modifications were incorporated into the sequence employing a double coupling step with a coupling time of 11 minutes per each coupling (total coupling time 22 min). The oxidizer contact time was set to 1.2 min and thiolation time was 5.2 min.

Sequences were synthesized with removal of the final DMT group, with exception of the MMT group from the NH2DEG sequences.

At the end of the synthesis, the oligonucleosides were cleaved from the solid support using a 1:1 volume solution of 28-30% ammonium hydroxide (Sigma-Aldrich, Cat. #221228) and 40% aqueous methylamine (Sigma-Aldrich, Cat. #8220911000) for 16 hours at 6° C. The solid support was then filtered off, the filter was thoroughly washed with H₂O and the volume of the combined solution was reduced by evaporation under reduced pressure. The pH of the resulting solution was adjusted to pH 7 with 10% AcOH (Sigma-Aldrich, Cat. #A6283).

The crude materials were purified either by reversed phase (RP) HPLC or anion exchange (AEX) HPLC.

RP HPLC purification was performed using a XBridge C18 Prep 19×50 mm column (Waters) on an AKTA Pure instrument (GE Healthcare). Buffer A was 100 mM triethyl-ammonium acetate (TEAAc, Biosolve) pH 7 and buffer B contained 95% acetonitrile in buffer A. A flow rate of 10 mL/min and a temperature of 60° C. were employed. UV traces at 280 nm were recorded. A gradient of 0% B to 100% B within 120 column volumes was employed. Appropriate fractions were pooled and precipitated in the freezer with 3 M sodium acetate (NaOAc) (Sigma-Aldrich), pH 5.2 and 85% ethanol (VWR). Pellets were isolated by centrifugation, redissolved in water (50 mL), treated with 5 M NaCl (5 mL) and desalted by Size exclusion HPLC on an Akta Pure instrument using a 50×165 mm ECO column (YMC, Dinslaken, Germany) filled with Sephadex G25-Fine resin (GE Healthcare).

AEX HPLC purification was performed using a TSK gel SuperQ-5PW 20×200 mm (BISCHOFF Chromatography) on an AKTA Pure instrument (GE Healthcare). Buffer A was 20 mM sodium phosphate (Sigma-Aldrich) pH 7.8 and buffer B was the same as buffer A with the addition of 1.4 M sodium bromide (Sigma-Aldrich). A flow rate of 10 mL/min and a temperature of 60° C. were employed. UV traces at 280 nm were recorded. A gradient of 10% B to 100% B within 27 column volumes was employed. Appropriate fractions were pooled and precipitated in the freezer with 3 M NaOAc, pH 5.2 and 85% ethanol. Pellets were isolated by centrifugation, redissolved in water (50 mL), treated with 5 M NaCl (5 mL) and desalted by size exclusion chromatography.

The MMT group was removed with 25% acetic acid in water. Once the reaction was complete the solution was neutralized and the samples were desalted by size exclusion chromatography.

Single strands were analyzed by analytical LC-MS on a 2.1×50 mm XBridge C18 column (Waters) on a Dionex Ultimate 3000 (Thermo Fisher Scientific) HPLC system combined either with a LCQ Deca XP-plus Q-ESI-TOF mass spectrometer (Thermo Finnigan) or with a Compact ESI-Qq-TOF mass spectrometer (Bruker Daltonics). Buffer A was 16.3 mM triethylamine, 100 mM 1,1,1,3,3,3-hexafluoro-2-propanol (HFIP) and 1% MeOH in H₂O and buffer B contained buffer A in 95% MeOH. A flow rate of 250 μL/min and a temperature of 60° C. were employed. UV traces at 260 and 280 nm were recorded. A gradient of 1-40% B within 0.5 min followed by 40 to 100% B within 13 min was employed. Methanol (LC-MS grade), water (LC-MS grade), 1,1,1,3,3,3-hexafluoro-2-propanol (puriss. p.a.) and triethylamine (puriss. p.a.) were purchased from Sigma-Aldrich.

### x) TriGalNAc Tether 2 (GalNAc-T2) Conjugation at 5′-End or 3′-End

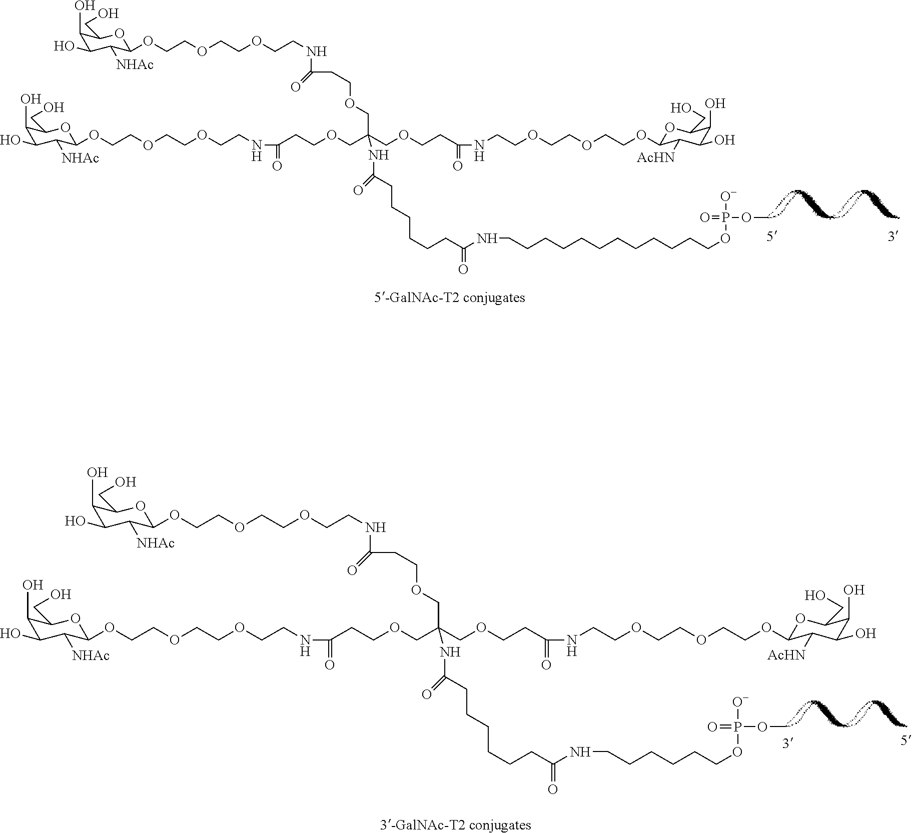 [SMILES: COP(=O)([O-])OCCCCCCCCCCCCNC(=O)CCCCCCC(=O)NC(COCCC(=O)NCCOCCOCCOC1OC(CO)C(O)C(O)C1NC(C)=O)(COCCC(=O)NCCOCCOCCOC1OC(CO)C(O)C(O)C1NC(C)=O)COCCC(=O)NCCOCCOCCOC1OC(CO)C(O)C(O)C1NC(C)=O ; COP(=O)([O-])OCCCCCCNC(=O)CCCCCCC(=O)NC(COCCC(=O)NCCOCCOCCOC1OC(CO)C(O)C(O)C1NC(C)=O)(COCCC(=O)NCCOCCOCCOC1OC(CO)C(O)C(O)C1NC(C)=O)COCCC(=O)NCCOCCOCCOC1OC(CO)C(O)C(O)C1NC(C)=O]

Preparation of TriGalNAc tether 2 NHS ester: To a solution of carboxylic acid tether 2 (compound 15, 227 mg, 121 μmol) in DMF (2.1 mL), N-hydroxysuccinimide (NHS) (15.3 mg, 133 μmol) and N,N′-diisopropylcarbodiimide (DIC) (19.7 μL, 127 μmol) were added. The solution was stirred at room temperature for 18 h and used without purification for the subsequent conjugation reactions.

General procedure for triGalNAc tether 2 conjugation: Amine-modified single strand was dissolved at 700 OD/mL in 50 mM carbonate/bicarbonate buffer pH 9.6/DMSO 4:6 (v/v) and to this solution was added one molar equivalent of Tether 2 NHS ester (57 mM) solution in DMF. The reaction was carried out at room temperature and after 1 h another molar equivalent of the NHS ester solution was added. The reaction was allowed to proceed for one more hour and reaction progress was monitored by LCMS. At least two molar equivalent excess of the NHS ester reagent relative to the amino modified oligonucleoside were needed to achieve quantitative consumption of the starting material. The reaction mixture was diluted 15-fold with water, filtered once through 1.2 μm filter from Sartorius and then purified by reserve phase (RP HPLC) on an Akta Pure (GE Healthcare) instrument.

The purification was performed using a XBridge C18 Prep 19×50 mm column from Waters. Buffer A was 100 mM TEEAc pH 7 and buffer B contained 95% acetonitrile in buffer A. A flow rate of 10 mL/min and a temperature of 60° C. were employed. UV traces at 280 nm were recorded. A gradient of 0-100% B within 60 column volumes was employed.

Fractions containing full-length conjugated oligonucleosides were pooled together, precipitated in the freezer with 3 M NaOAc, pH 5.2 and 85% ethanol and then dissolved at 1000 OD/mL in water. The O-acetates were removed with 20% ammonium hydroxide in water until completion (monitored by LC-MS).

The conjugates were desalted by size exclusion chromatography using Sephadex G25 Fine resin (GE Healthcare) on an Akta Pure (GE Healthcare) instrument to yield the conjugated oligonucleosides in an isolated yield of 60-80%.

TABLE 20

| Sense | | Purity by RP |
|---|---|---|
| strand ID | Sense strand sequence 5′-3′ | HPLC (%) |
| X91409 | (GalNAc-T2)(NH2C12)gacuuuCfaUfCfCfuggaaauasusa(invabasic) | 85.0 |
| | (invabasic) | |
| X91410 | (GalNAc-T2)(NH2C12)aaGfcAfaGfaUfAfUfuUfuuAfuAfasusa | 92.3 |
| | (invabasic)(invabasic) | |
| X91411 | (GalNAc-T2)(NH2C12)ugggauUfuCfAfUfguaaccaasgsa(invabasic) | 92.7 |
| | (invabasic) | |
| X91433 | (invabasic)(invabasic)gsascuuuCfaUfCfCfuggaaauasusa(NHC6) | 85.3 |
| | (GalNAc-T2) | |
| X91434 | (invabasic)(invabasic)asasGfcAfaGfaUfAfUfuUfuuAfuAfaua | 85.8 |
| | (NHC6)(GalNAc-T2) | |
| X91435 | (invabasic)(invabasic)usgsggauUfuCfAfUfguaaccaaga(NHC6) | 84.0 |
| | (GalNAc-T2) | |

The conjugates were characterized by HPLC-MS analysis with a 2.1×50 mm XBridge C18 column (Waters) on a Dionex Ultimate 3000 (Thermo Fisher Scientific) HPLC system equipped with a Compact ESI-Qq-TOF mass spectrometer (Bruker Daltonics). Buffer A was 16.3 mM triethylamine, 100 mM HFIP in 1% MeOH in H₂O and buffer B contained 95% MeOH in buffer A. A flow rate of 250 μL/min and a temperature of 60° C. were employed. UV traces at 260 and 280 nm were recorded. A gradient of 1-100% B within 31 min was employed.

### xi) Duplex Annealing

To generate the desired siRNA duplex, the two complementary strands were annealed by combining equimolar aqueous solutions of both strands. The mixtures were placed into a water bath at 70° C. for 5 minutes and subsequently allowed to cool to ambient temperature within 2 h. The duplexes were lyophilized for 2 days and stored at −20° C.

The duplexes were analyzed by analytical SEC HPLC on Superdex™ 75 Increase 5/150 GL column 5×153-158 mm (Cytiva) on a Dionex Ultimate 3000 (Thermo Fisher Scientific) HPLC system. Mobile phase consisted of 1×PBS containing 10% acetonitrile. An isocratic gradient was run in 10 min at a flow rate of 1.5 mL/min at room temperature. UV traces at 260 and 280 nm were recorded. Water (LC-MS grade) was purchased from Sigma-Aldrich and Phosphate-buffered saline (PBS; 10×, pH 7.4) was purchased from GIBCO (Thermo Fisher Scientific).

GalNAc conjugates prepared are compiled in the table below. These were directed against 3 different target genes. siRNA coding along with the corresponding single strands, sequence information as well as purity for the duplexes is captured.

TABLE 21

| | | | | Duplex |
|---|---|---|---|---|
| | Duplex | ssRN | | Purity by |
| Target | ID | ID | SSRNA-Sequence 5′-3′ | HPLC (%) |
| GO | ETX0 | X914 | (GalNAc-T2)(NH2C12)gacuuuCfaUfCfCfuggaaauasusa | 94.1 |
| | 02 | 09 | (invabasic)(invabasic) | |
| | | X384 | usAfsuauUfuCfCfaggaUfgAfaagucscsa | |
| | | 83 | | |
| | ETX0 | X914 | (invabasic)(invabasic)gsascuuuCfaUfCfCfuggaaauasusa | 94.1 |
| | 06 | 33 | (NHC6)(GalNAc-T2) | |
| | | X384 | usAfsuauUfuCfCfaggaUfgAfaagucscsa | |
| | | 83 | | |
| C5 | ETX0 | X914 | (GalNAc-T2)(NH2C12)aaGfcAfaGfaUfAfUfuUfuuAfuAfasusa | 93.5 |
| | 11 | 10 | (invabasic)(invabasic) | |
| | | X913 | usAfsUfuAfuaAfaAfauaUfcUfuGfcuususudTdT | |
| | | 81 | | |
| | ETX0 | X914 | (invabasic)(invabasic)asasGfcAfaGfaUfAfUfuUfuuAfuAf | 95.3 |
| | 15 | 34 | aua(NHC6)(GalNAc-T2) | |
| | | X913 | usAfsUfuAfuaAfaAfauaUfcUfuGfcuususudTdT | |
| | | 81 | | |
| | ETXO | X914 | (GalNAc-T2)(NH2C12)ugggauUfuCfAfUfguaaccaasgsa | 97.5 |
| | 20 | 11 | (invabasic)(invabasic) | |
| | | X381 | usCfsuugGfuuAfcaugAfaAfucccasusc | |
| | | 04 | | |
| TTR | ETXO | X914 | (invabasic)(invabasic)usgsggauUfuCfAfUfguaaccaaga | 95.3 |
| | 24 | 35 | (NHC6)(GalNAc-T2) | |
| | | X381 | usCfsuugGfuuAfcaugAfaAfucccasusc | |
| | | 04 | | |

The following schemes further set out the routes of synthesis:

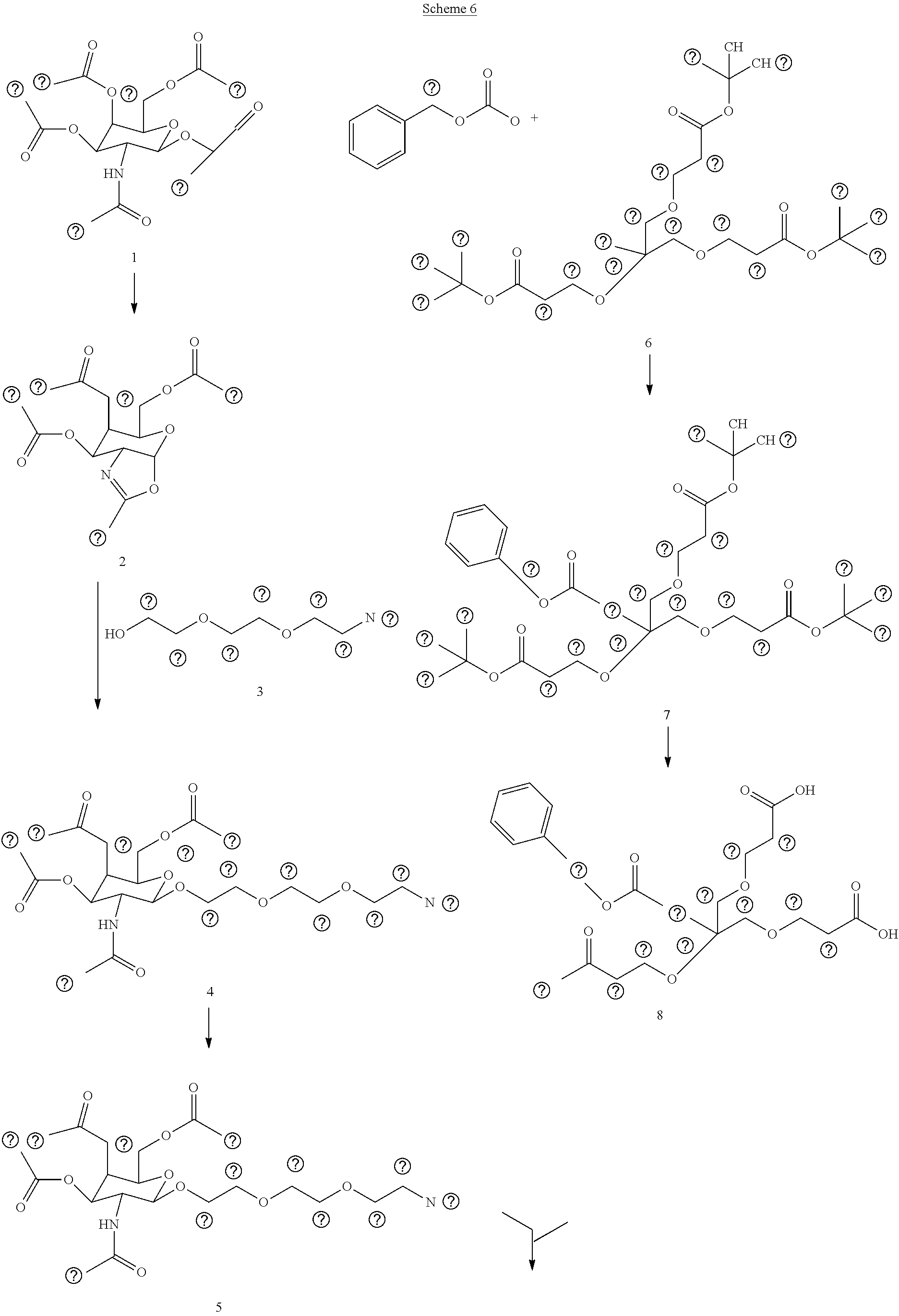

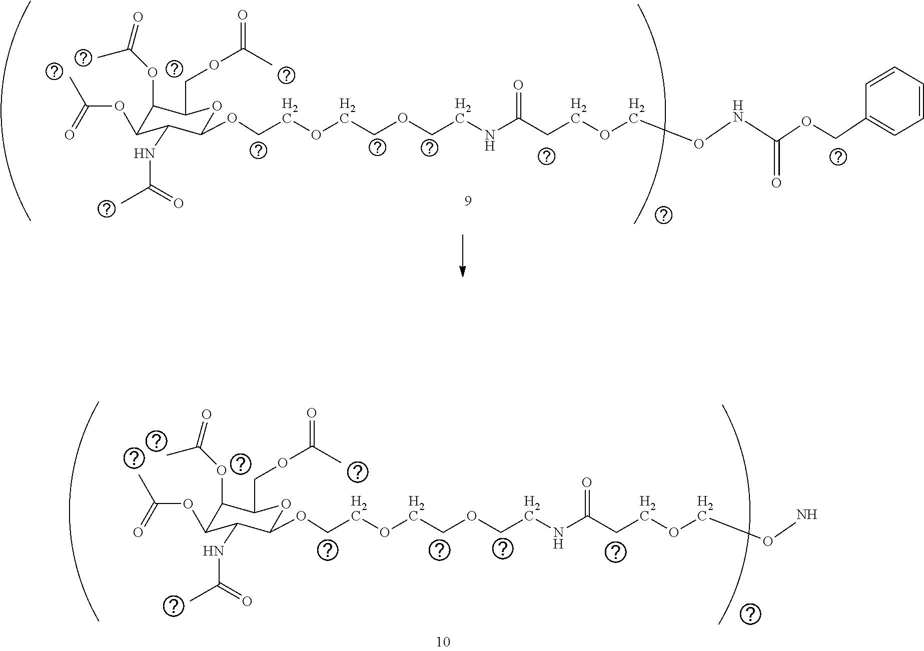

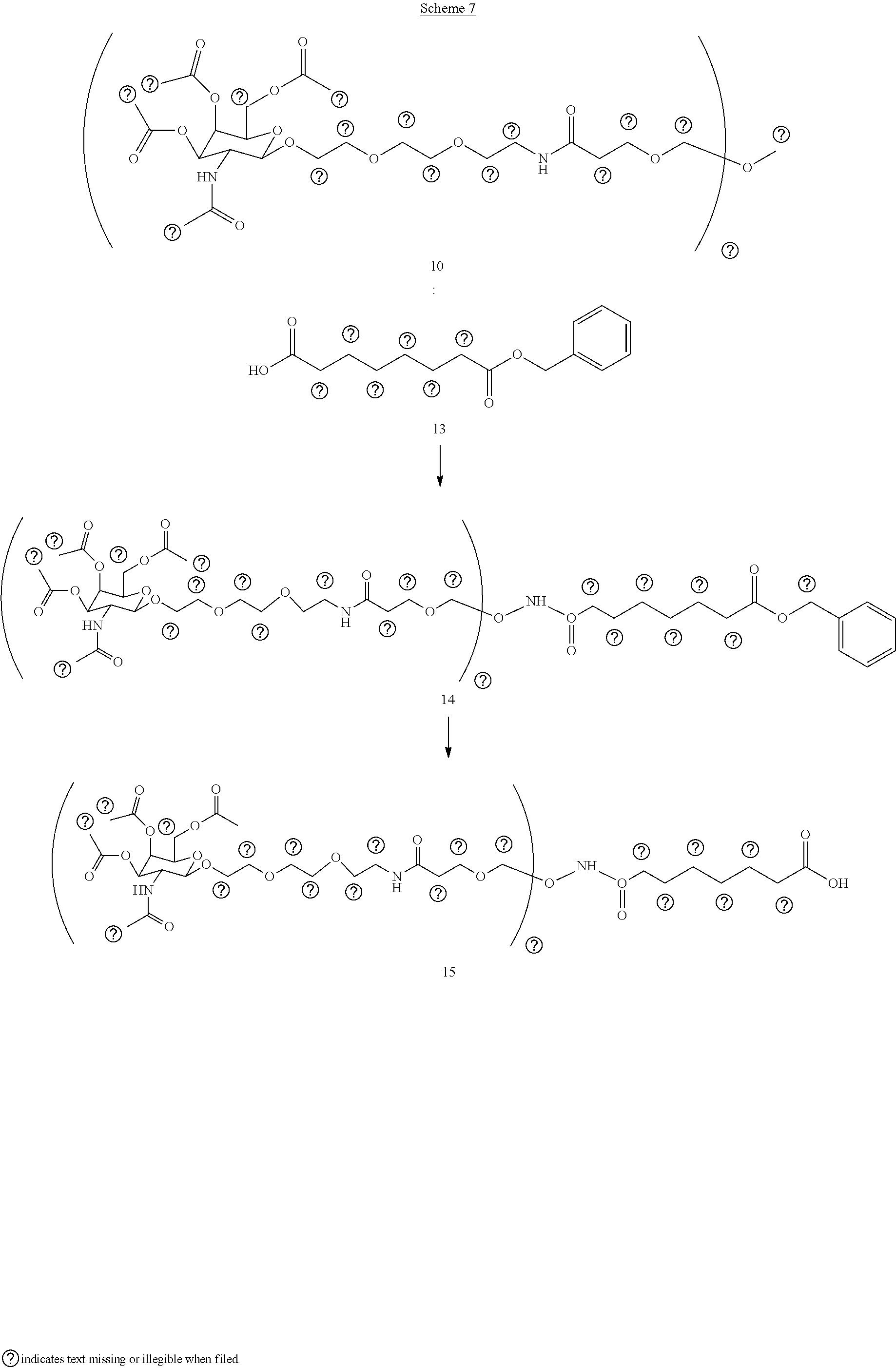

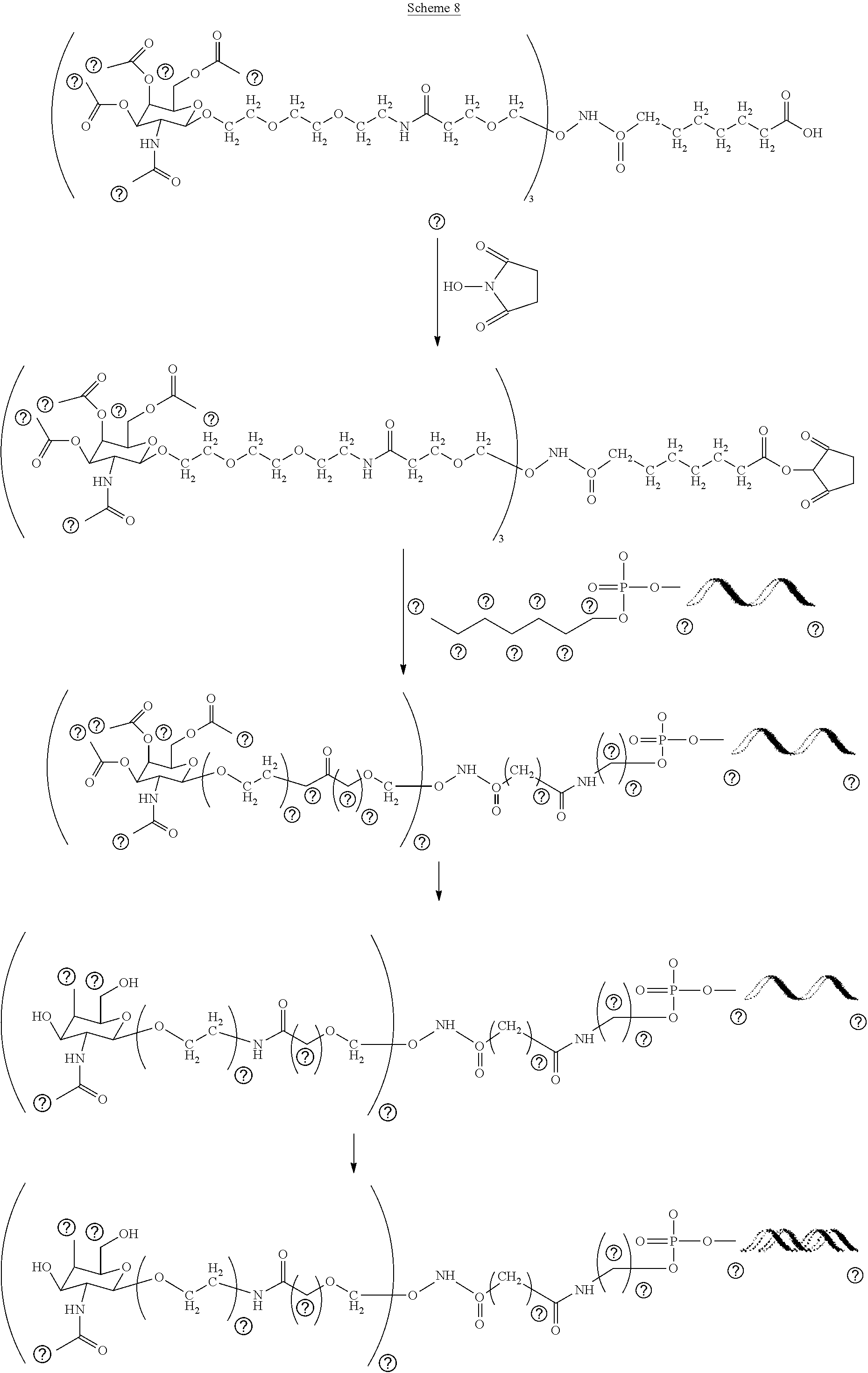

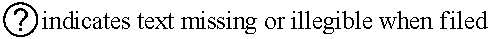

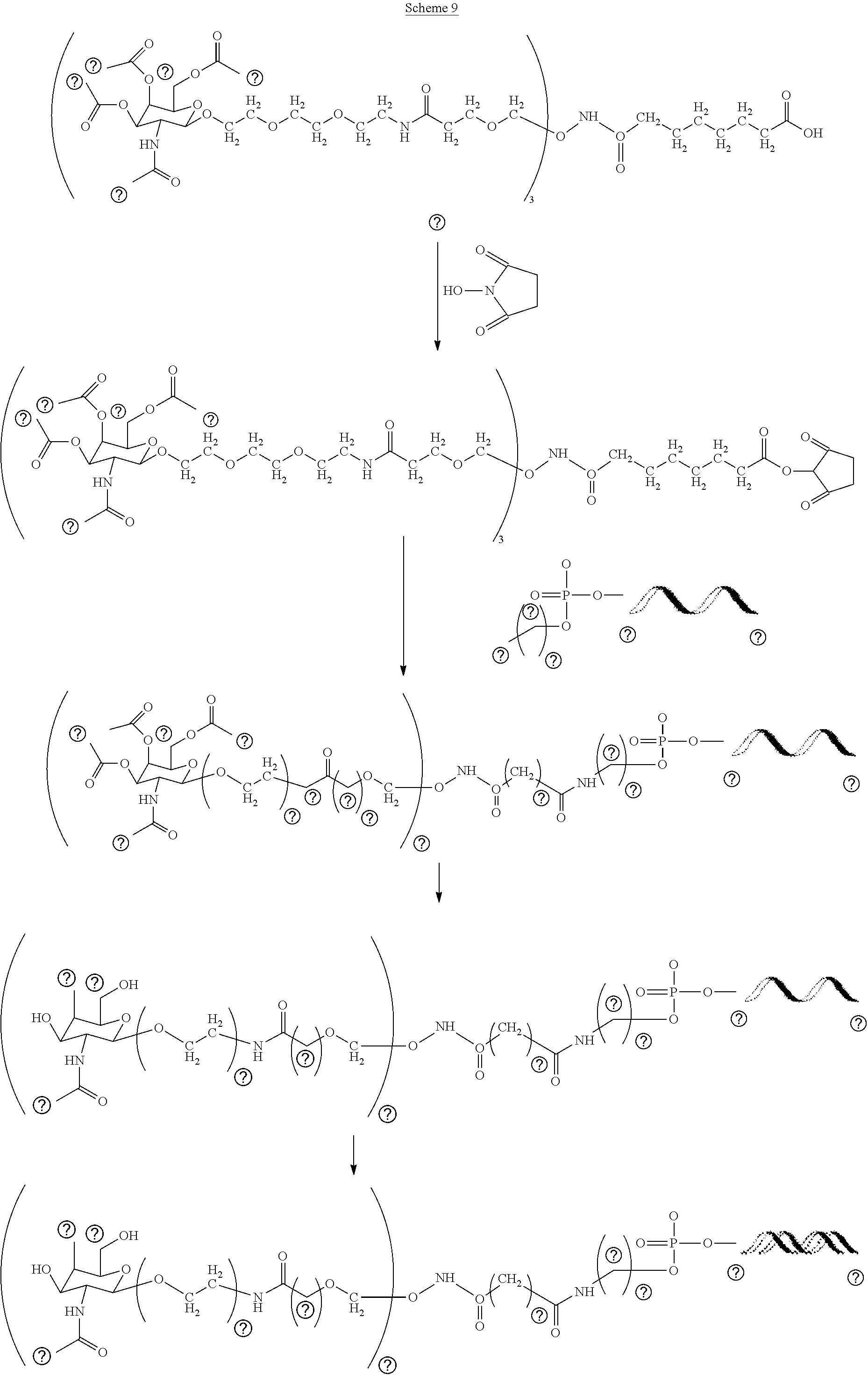

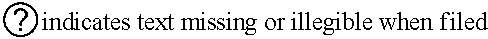

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

### Example 5

### Mouse Data for GalNAc-siRNA Constructs ETX005, ETX006, ETX014 and ETX015

ETX005 (Targeting HAO1 mRNA) T1a Inverted Abasic

An in vivo mouse pharmacology study was performed showing knockdown of HAO1 mRNA in liver tissue with an associated increase in serum glycolate level following a single subcutaneous dose of up to 3 mg/kg GalNAc conjugated modified siRNA ETX005.

Male C57BL/6 mice with an age of about 8 weeks were randomly assigned into groups of 21 mice. On day 0 of the study, the animals received a single subcutaneous dose of 0.3 or 3 mg/kg GalNAc-siRNA dissolved in saline (sterile 0.9% sodium chloride) or saline only as control. At day 1, day 2, day 4, day 7, day 14, day 21, and day 28 of the study, 3 mice from each group were euthanised and serum and liver samples taken.

Serum was taken from a group of 5 untreated mice at day 0 to provide a baseline measurement of glycolate concentration.

Serum was stored at −80° C. until further analysis. Liver sample (approximately 50 mg) were treated with RNAlater and stored overnight at 4° C., before being stored at −80° C.

Liver samples were analysed using quantitative real-time PCR for HAO1 mRNA (Thermo assay ID Mm00439249_m1) and the housekeeping gene GAPDH mRNA (Thermo assay ID Mm99999915_g1). The delta delta Ct method was used to calculated changes in HAO1 expression normalised to GAPDH and relative to the saline control group.

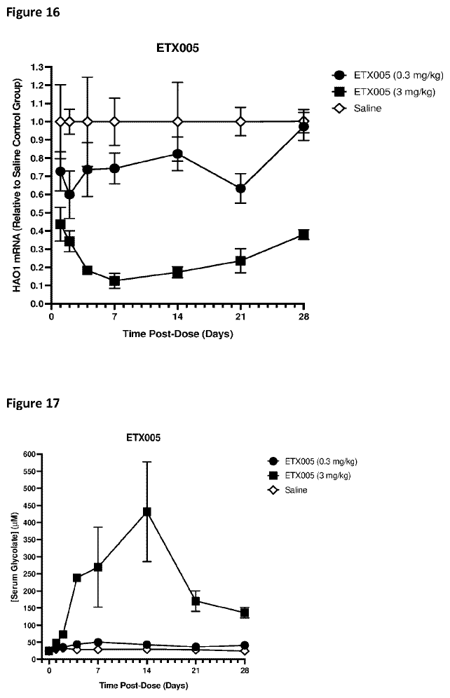

A single 3 mg/kg dose of ETX005 inhibited HAO1 mRNA expression by greater than 80% after 7 days (FIG. 16). The suppression of HAO1 expression was durable, with a single 3 mg/kg dose of ETX005 maintaining greater than 60% inhibition of HAO1 mRNA at the end of the study on day 28. A single dose of 0.3 mg/kg ETX005 also inhibited HAO1 expression when compared with the saline control group, with HAO1 expression levels reaching normal levels only at day 28 of the study.

Suppression of HAO1 mRNA expression is expected to cause an increase in serum glycolate levels. Serum glycolate concentration was measured using LC-MS/MS (FIG. 17). A single 3 mg/kg dose of ETX005 caused a significant increase in serum glycolate concentration, reaching peak levels 14 days after dosing and remaining higher than baseline level (day 0) and the saline control group until the end of the study at day 28. A single 0.3 mg/kg dose of ETX005 showed a smaller and more transient increase in serum glycolate concentration above the level seen in a baseline and saline control group, demonstrating that a very small dose can suppress HAO1 mRNA at a magnitude sufficient to affect the concentration of a metabolic biomarker in serum.

FIG. 16. Single dose mouse pharmacology of ETX005. HAO1 mRNA expression is shown relative to the saline control group. Each point represents the mean and standard deviation of 3 mice.

FIG. 17. Single dose mouse pharmacology of ETX005. Serum glycolate concentration is shown. Each point represents the mean and standard deviation of 3 mice, except for baseline glycolate concentration (day 0) which was derived from a group of 5 mice.

ETX006 (Targeting HAO1 mRNA) T2a Inverted Abasic

An in vivo mouse pharmacology study was performed showing knockdown of HAO1 mRNA in liver tissue and a concomitant increase in serum glycolate levels following a single subcutaneous dose of up to 3 mg/kg GalNAc conjugated modified siRNA ETX006.

Male C57BL/6 mice with an age of about 8 weeks were randomly assigned into groups of 21 mice. On day 0 of the study, the animals received a single subcutaneous dose of 0.3 or 3 mg/kg GalNAc-siRNA dissolved in saline (sterile 0.9% sodium chloride) or saline only as control. At day 1, day 2, day 4, day 7, day 14, day 21, and day 28 of the study, 3 mice from each group were euthanised and serum and liver samples taken.

Serum was taken from a group of 5 untreated mice at day 0 to provide a baseline measurement of glycolate concentration.

Serum was stored at −80° C. until further analysis. Liver sample (approximately 50 mg) were treated with RNAlater and stored overnight at 4° C., before being stored at −80° C.

Liver samples were analysed using quantitative real-time PCR for HAO1 mRNA (Thermo assay ID Mm00439249_m1) and the housekeeping gene GAPDH mRNA (Thermo assay ID Mm99999915_g1). The delta delta Ct method was used to calculated changes in HAO1 expression normalised to GAPDH and relative to the saline control group.

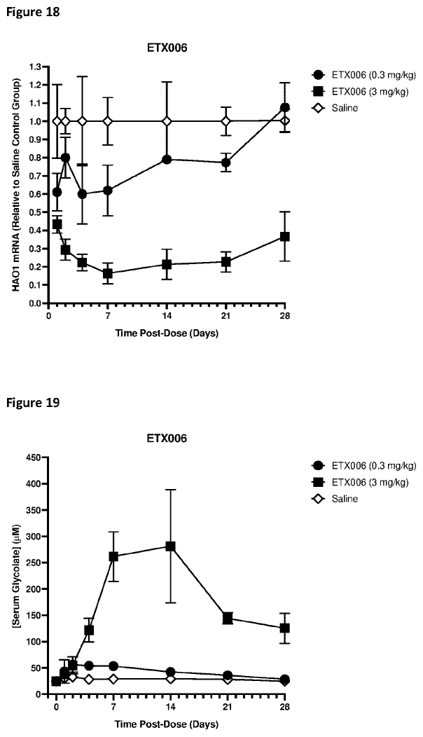

A single 3 mg/kg dose of ETX006 inhibited HAO1 mRNA expression by than 80% after 7 days (FIG. 18). The suppression of HAO1 expression was durable and continued until the end of the study, with ETX006 maintaining greater than 60% inhibition of HAO1 mRNA at day 28. A single dose of 0.3 mg/kg also inhibited HAO1 expression when compared with the saline control group, with HAO1 expression levels reaching normal levels only at day 28 of the study.

Suppression of HAO1 mRNA expression is expected to cause an increase in serum glycolate levels. Serum glycolate concentration was measured using LC-MS/MS (FIG. 19). A single 3 mg/kg dose of ETX006 caused a significant increase in serum glycolate concentration, reaching peak levels 14 days after dosing and remaining higher than baseline levels (day 0) and the saline control group until the end of the study at day 28. A single 0.3 mg/kg dose of ETX006 showed a smaller and more transient increase in serum glycolate concentration above the level seen in a baseline and saline control groups, demonstrating that a very small dose can also affect the concentration of a metabolic biomarker in serum.

FIG. 18. Single dose mouse pharmacology of ETX006. HAO1 mRNA expression is shown relative to the saline control group. Each point represents the mean and standard deviation of 3 mice.

FIG. 19. Single dose mouse pharmacology of ETX006. Serum glycolate concentration is shown. Each point represents the mean and standard deviation of 3 mice, except for baseline glycolate concentration (day 0) which was derived from a group of 5 mice.

ETX014 (Targeting C5 mRNA) T1a Inverted Abasic

An in vivo mouse pharmacology study was performed showing knockdown of C5 mRNA in liver tissue and the resulting decrease in serum C5 protein concentration following a single subcutaneous dose of up to 3 mg/kg GalNAc conjugated modified siRNA ETX014.

Male C57BL/6 mice with an age of about 8 weeks were randomly assigned into groups of 21 mice. On day 0 of the study, the animals received a single subcutaneous dose of 0.3, 1, or 3 mg/kg GalNAc-siRNA dissolved in saline (sterile 0.9% sodium chloride) or saline only as control. At day 1, day 2, day 4, day 7, day 14, day 21, and day 28 of the study, 3 mice from each group were euthanised and serum and liver samples taken.

Serum was stored at −80° C. until further analysis. Liver sample (approximately 50 mg) were treated with RNAlater and stored overnight at 4° C., before being stored at −80° C.

Liver samples were analysed using quantitative real-time PCR for C5 mRNA (Thermo assay ID Mm00439275_m1) and the housekeeping gene GAPDH mRNA (Thermo assay ID Mm99999915_g1). The delta delta Ct method was used to calculated changes in C5 expression normalised to GAPDH and relative to the saline control group.

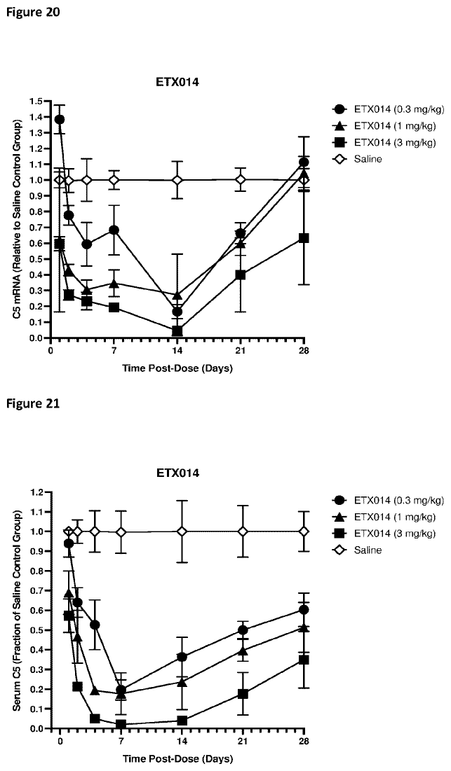

ETX014 inhibited C5 mRNA expression in a dose-dependent manner (FIG. 20) with the 3 mg/kg dose achieving greater than 90% reduction in C5 mRNA at day 14. The suppression of C5 expression by ETX014 was durable, with the 3 mg/kg dose of each molecule showing clear knockdown of C5 mRNA until the end of the study at day 28.

For C5 protein level analysis, serum samples were measured using a commercially available C5 ELISA kit (Abcam ab264609). Serum C5 levels were calculated relative to the saline group means at matching timepoints.

Serum protein data support the mRNA analysis (FIG. 21). Treatment with ETX014 caused a dose-dependent decrease in serum C5 protein concentration. All doses of ETX014 reduced C5 protein levels by greater than 70%, with the 3 mg/kg dose reducing C5 levels to almost undetectable levels at day 7 of the study. Reduction of serum C5 was sustained by all doses until study termination, with even the lowest dose of 0.3 mg/kg still showing inhibition of approximately 40% at day 28.

FIG. 20. Single dose mouse pharmacology of ETX014. C5 mRNA expression is shown relative to the saline control group. Each point represents the mean and standard deviation of 3 mice.

FIG. 21. Single dose mouse pharmacology of ETX0014. Serum C5 concentration is shown relative to the saline control group. Each point represents the mean and standard deviation of 3 mice.

ETX015 (Targeting C5 mRNA) T2a Inverted Abasic

An in vivo mouse pharmacology study was performed showing knockdown of C5 mRNA in liver tissue and the resulting decrease in serum C5 protein concentration following a single subcutaneous dose of up to 3 mg/kg GalNAc conjugated modified siRNA ETX015.

Male C57BL/6 mice with an age of about 8 weeks were randomly assigned into groups of 21 mice. On day 0 of the study, the animals received a single subcutaneous dose of 0.3, 1, or 3 mg/kg GalNAc-siRNA dissolved in saline (sterile 0.9% sodium chloride) or saline only as control. At day 1, day 2, day 4, day 7, day 14, day 21, and day 28 of the study, 3 mice from each group were euthanised and serum and liver samples taken.

Serum was stored at −80° C. until further analysis. Liver sample (approximately 50 mg) were treated with RNAlater and stored overnight at 4° C., before being stored at −80° C.

Liver samples were analysed using quantitative real-time PCR for C5 mRNA (Thermo assay ID Mm00439275_m1) and the housekeeping gene GAPDH mRNA (Thermo assay ID Mm99999915_g1). The delta delta Ct method was used to calculated changes in C5 expression normalised to GAPDH and relative to the saline control group.

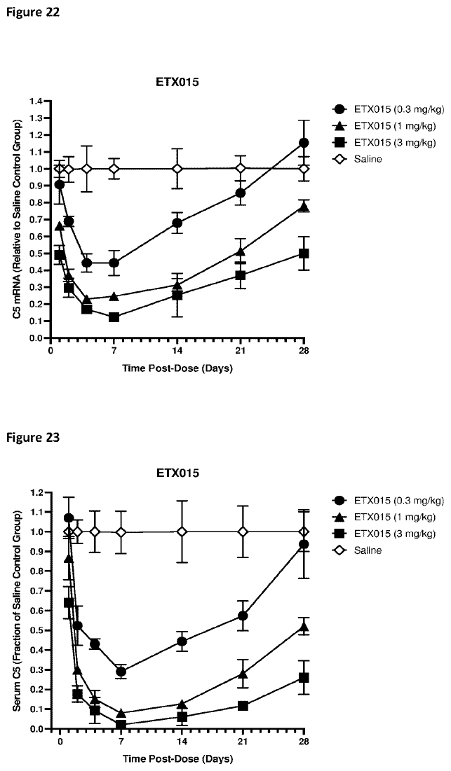

ETX015 inhibited C5 mRNA expression in a dose-dependent manner (FIG. 22) with the 3 mg/kg dose achieving greater than 85% reduction of C5 mRNA. The suppression of C5 expression was durable, with the 3 mg/kg dose of each molecule showing clear knockdown of C5 mRNA until the end of the study at day 28. Mice dosed with 3 mg/kg ETX015 still exhibited less than 50% of normal liver C5 mRNA levels 28 days after dosing.

For C5 protein level analysis, serum samples were measured using a commercially available C5 ELISA kit (Abcam ab264609). Serum C5 levels were calculated relative to the saline group means at matching timepoints.

Serum protein data support the mRNA analysis (FIG. 23). ETX015 caused a dose-dependent decrease in serum C5 protein concentration. The 3 mg/kg and 1 mg/kg doses of ETX015 achieved greater than 90% reduction of serum C5 protein levels. The highest dose exhibited durable suppression of C5 protein expression, with a greater than 70% reduction of C5 at day 28 of the study compared to saline control.

FIG. 22. Single dose mouse pharmacology of ETX015. C5 mRNA expression is shown relative to the saline control group. Each point represents the mean and standard deviation of 3 mice.

FIG. 23. Single dose mouse pharmacology of ETX0014. Serum C5 concentration is shown relative to the saline control group. Each point represents the mean and standard deviation of 3 mice.

### Example 6

### NHP Data for GalNAc-siRNA Constructs ETX023 and ETX024

ETX023 (Targeting TTR mRNA) Tia Inverted Abasic

ETX023 pharmacology was evaluated in non-human primate (NHP) by quantifying serum transthyretin (TTR) protein levels. A single subcutaneous dose of 1 mg/kg GalNAc conjugated modified siRNA ETX023 demonstrated durable suppression of TTR protein expression.

Male cynomolgus monkeys (3-5 years old, 2-3 kg) were assigned into groups of 3 animals. Animals were acclimatised for 2 weeks, and blood taken 14 days prior to dosing to provide baseline TTR concentration. A liver biopsy was performed 18 or 38 days prior to dosing to provide baseline mRNA levels. On day 0 of the study, the animals received a single subcutaneous dose of 1 mg/kg GalNAc-siRNA ETX023 dissolved in saline (sterile 0.9% sodium chloride). At day 3, day 14, day 28, day 42, day 56, day 70 and day 84 of the study, a liver biopsy was taken and RNA extracted for measurement of TTR mRNA. At day 1, day 3, day 7, day 14, day 28, day 42, day 56, day 70 and day 84 of the study, a blood sample was taken for measurement of serum TTR concentration and clinical blood chemistry analysis.

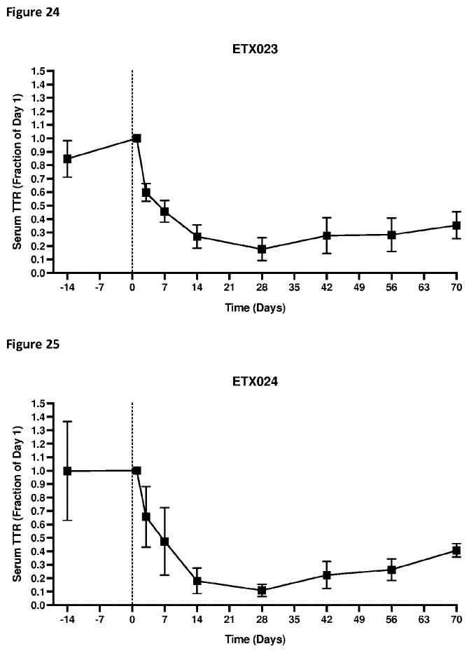

Suppression of TTR mRNA expression is expected to cause a decrease in serum TTR protein levels. Serum TTR protein concentration was measured by a commercially available ELISA kit (Abcam ab231920). TTR concentration as a fraction of day 1 was calculated for each individual animal and this was plotted as mean and standard deviation for the group of 3 animals (FIG. 24).

A single 1 mg/kg dose of ETX023 caused a rapid and significant reduction in serum TTR concentration, reaching nadir 28 days after dosing and remaining suppressed until day 70.

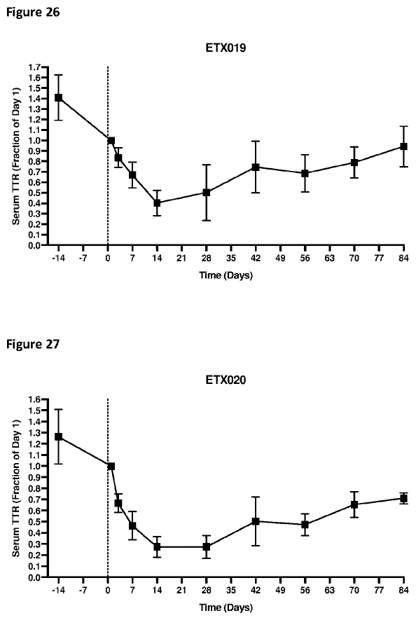

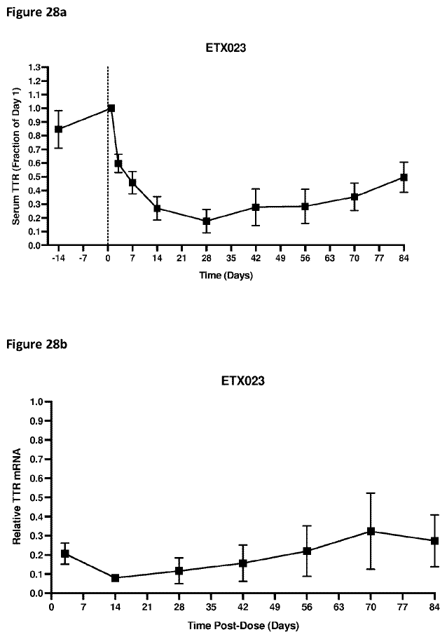

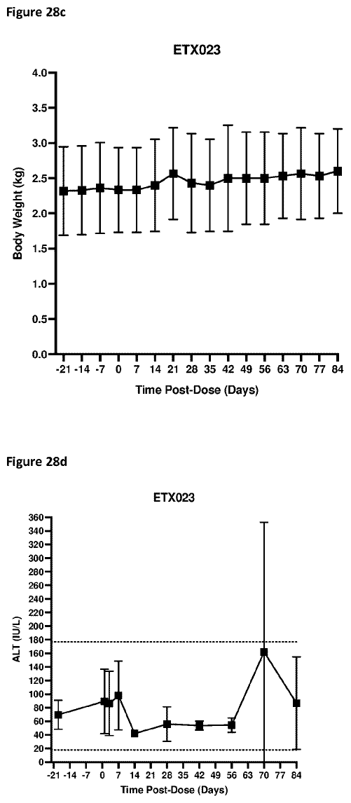

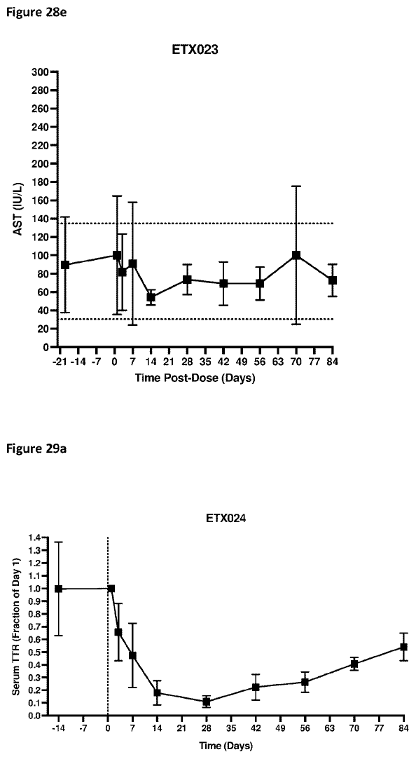

Data was further obtained until day 84. Identical experiments were carried out using ETX019. Data is provided for 84 days in FIG. 26 for ETX0019 and FIG. 28a for ETX0023.

TTR mRNA was measured by real-time quantitative PCR using a TaqMan Gene expression kit TTR (Thermo, assay ID Mf02799963_m1). GAPDH expression was also measured (Thermo, assay ID Mf04392546_g1) to provide a reference. Relative TTR expression for each animal was calculated normalised to GAPDH and relative to pre-dose levels by the □□Ct method. A single 1 mg/kg dose of ETX023 also caused a rapid and significant reduction in liver TTR mRNA, reaching nadir 14 days after dosing and remaining suppressed until day 84 (FIG. 28b).

Animal body weight was measured once a week during the study. No fluctuations or decrease in body weight was associated with dosing ETX023 and animals continued to gain weight throughout the study (FIG. 28c).

Serum was analysed within 2 hours using an automatic biochemical analyser. A significant increase in ALT (alanine transaminase) and AST (aspartate transaminase) are commonly used to demonstrate liver toxicity. No increase in ALT (FIG. 28d) or ALT (FIG. 28e) was associated with dosing of ETX023.

ETX024 (Targeting TTR mRNA) T2a Inverted Abasic

ETX024 pharmacology was evaluated in non-human primate (NHP) by quantifying serum transthyretin (TTR) protein levels. A single subcutaneous dose of 1 mg/kg GalNAc conjugated modified siRNA ETX024 demonstrated durable suppression of TTR protein expression.

Male cynomolgus monkeys (3-5 years old, 2-3 kg) were assigned into groups of 3 animals. Animals were acclimatised for 2 weeks, and blood taken 14 days prior to dosing to provide baseline TTR concentration. A liver biopsy was performed 18 or 38 days prior to dosing to provide baseline mRNA levels. On day 0 of the study, the animals received a single subcutaneous dose of 1 mg/kg GalNAc-siRNA ETX024 dissolved in saline (sterile 0.9% sodium chloride). At day 3, day 14, day 28, day 42, day 56, day 70 and day 84 of the study, a liver biopsy was taken and RNA extracted for measurement of TTR mRNA. At day 1, day 3, day 7, day 14, day 28, day 42, day 56, 70 and day 84 of the study, a blood sample was taken for measurement of serum TTR concentration and clinical blood chemistry analysis.

Suppression of TTR mRNA expression is expected to cause a decrease in serum TTR protein levels. Serum TTR protein concentration was measured by a commercially available ELISA kit (Abeam ab231920). TTR concentration as a fraction of day 1 was calculated for each individual animal and this was plotted as mean and standard deviation for the group of 3 animals (FIG. 25).

A single 1 mg/kg dose of ETX024 caused a rapid and significant reduction in serum TTR concentration, reaching nadir 28 days after dosing and remaining suppressed until day 70.

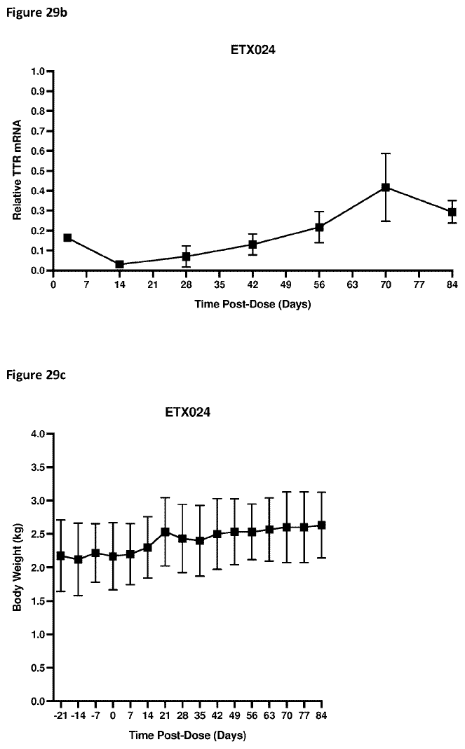

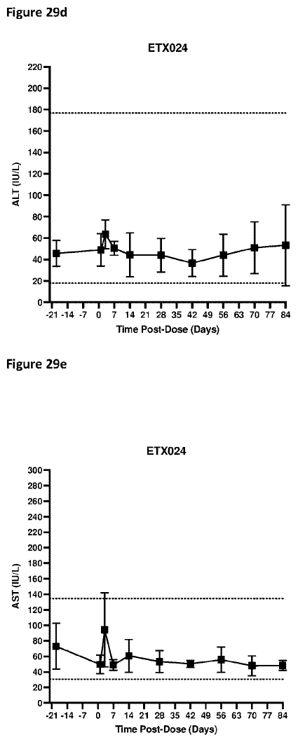

Data was further obtained with ETX024 until day 84. Identical experiments were carried out using ETX020. Data is provided for 84 days in FIG. 27 for ETX0020 and FIG. 29a for ETX0024.

TTR mRNA was measured by real-time quantitative PCR using a TaqMan Gene expression kit TTR (Thermo, assay ID Mf02799963_m1). GAPDH expression was also measured (Thermo, assay ID Mf04392546_g1) to provide a reference. Relative TTR expression for each animal was calculated normalised to GAPDH and relative to pre-dose levels by the □□Ct method. A single 1 mg/kg dose of ETX024 caused a rapid and significant reduction in liver TTR mRNA, reaching nadir 14 days after dosing and remaining suppressed until day 84 (FIG. 29b).

Animal body weight was measured once a week during the study. No fluctuations or decrease in body weight was associated with dosing ETX024 and animals continued to gain weight throughout the study (FIG. 29c).

Serum was analysed within 2 hours using an automatic biochemical analyser. A significant increase in ALT (alanine transaminase) and AST (aspartate transaminase) are commonly used to demonstrate liver toxicity. No increase in ALT (FIG. 29d) or ALT (FIG. 29e) was associated with dosing of ETX024.

In preferred aspects, compounds of the invention are able to depress serum protein level of a target protein to a value below the initial (starting) concentration at day 0, over a period of up to at least about 14 days after day 0, up to at least about 21 days after day 0, up to at least about 28 days after day 0, up to at least about 35 days after day 0, up to at least about 42 days after day 0, up to at least about 49 days after day 0, up to at least about 56 days after day 0, up to at least about 63 days after day 0, up to at least about 70 days after day 0, up to at least about 77 days after day 0, or up to at least about 84 days after day 0, hereinafter referred to as the “dose duration”. “Day 0” as referred to herein is the day when dosing of a compound of the invention to a patient is initiated, in other words the start of the dose duration or the time post dose.

In preferred aspects, compounds of the invention are able to depress serum protein level of a target protein to a value of at least about 90% or below of the initial (starting) concentration at day 0, such as at least about 85% or below, at least about 80% or below, at least about 75% or below, at least about 70% or below, at least about 65% or below, at least about 60% or below, at least about 55% or below, at least about 50% or below, at least about 45% or below, at least about 40% or below, at least about 35% or below, at least about 30% or below, at least about 25% or below, at least about 20% or below, at least about 15% or below, at least about 10% or below, at least about 5% or below, of the initial (starting) concentration at day 0. Typically such depression of serum protein can be maintained over a period of up to at least about 14 days after day 0, up to at least about 21 days after day 0, up to at least about 28 days after day 0, up to at least about 35 days after day 0, up to at least about 42 days after day 0, up to at least about 49 days after day 0, up to at least about 56 days after day 0, up to at least about 63 days after day 0, up to at least about 70 days after day 0, up to at least about 77 days after day 0, or up to at least about 84 days after day 0. More preferably, at a period of up to at least about 84 days after day 0, the serum protein can be depressed to a value of at least about 90% or below of the initial (starting) concentration at day 0, such as at least about 85% or below, at least about 80% or below, at least about 75% or below, at least about 70% or below, at least about 65% or below, at least about 60% or below, at least about 55% or below, at least about 50% or below, at least about 45% or below, at least about 40% or below, of the initial (starting) concentration at day 0.

In preferred aspects, compounds of the invention are able to achieve a maximum depression of serum protein level of a target protein to a value of at least about 50% or below of the initial (starting) concentration at day 0, such as at least about 45% or below, at least about 40% or below, at least about 35% or below, at least about 30% or below, at least about 25% or below, at least about 20% or below, at least about 15% or below, at least about 10% or below, at least about 5% or below, of the initial (starting) concentration at day 0. Typically such maximum depression of serum protein occurs at about day 14 after day 0, at about day 21 after day 0, at about day 28 after day 0, at about day 35 after day 0, or at about day 42 after day 0. More typically, such maximum depression of serum protein occurs at about day 14 after day 0, at about day 21 after day 0, or at about day 28 after day 0.

Specific compounds of the invention can typically achieve a maximum % depression of serum protein level of a target protein and/or a % depression over a period of up to at least about 84 days as follows:
ETX019 can typically achieve at least 50% depression of serum protein level of a target protein, typically TTR, typically at about 7 to 21 days after day 0, in particular at about 14 days after day 0, and/or can typically maintain at least 90% depression of serum protein level of a target protein, typically TTR, over a period of up to at least about 84 days after day 0 (as hereinbefore described, “day 0” as referred to herein is the day when dosing of a compound of the invention to a patient is initiated, and as such denotes the time post dose);
ETX020 can typically achieve at least 30% depression of serum protein level of a target protein, typically TTR, typically at about 7 to 21 days after day 0, in particular at about 14 days after day 0, and/or can typically maintain at least 80% depression of serum protein level of a target protein, typically TTR, over a period of up to at least about 84 days after day 0 (as hereinbefore described, “day 0” as referred to herein is the day when dosing of a compound of the invention to a patient is initiated, and as such denotes the time post dose);
ETX023 can typically achieve at least 20% depression of serum protein level of a target protein, typically TTR, typically at about 7 to 21 days after day 0, in particular at about 14 days after day 0, and/or can typically maintain at least 50% depression of serum protein level of a target protein, typically TTR, over a period of up to at least about 84 days after day 0 (as hereinbefore described, “day 0” as referred to herein is the day when dosing of a compound of the invention to a patient is initiated, and as such denotes the time post dose);
ETX024 can typically achieve at least 20% depression of serum protein level of a target protein, typically TTR, typically at about 7 to 21 days after day 0, in particular at about 14 days after day 0, and/or can typically maintain at least 60% depression of serum protein level of a target protein, typically TTR, over a period of up to at least about 84 days after day 0 (as hereinbefore described, “day 0” as referred to herein is the day when dosing of a compound of the invention to a patient is initiated, and as such denotes the time post dose).

Suitably the depression of serum level is determined in non-human primates by delivering a single subcutaneous dose of 1 mg/kg of the relevant active agent, eg ETX0023 or ETX0024, dissolved in saline (sterile 0.9% sodium chloride). Suitable methods are described herein. It will be appreciated that this is not limiting and other suitable methods with appropriate controls may be used.

### Example 7 ETX023 (Targeting TTR mRNA) T1a Inverted Abasic

### Total Bilirubin Levels Remained Stable Throughout the Study (FIG. 34)

Kidney health was monitored by assessment of urea (blood urea nitrogen, BUN) and creatinine concentration throughout the study. Both blood urea concertation (BUN) and creatinine levels remained stable and within the expected range after a single 1 mg/kg dose of ETX023 (FIGS. 35 and 36).

### Example 8 ETX024 (Targeting TTR mRNA) T2a Inverted Abasic

### Total Bilirubin Levels Remained Stable Throughout the Study (FIG. 37)

Kidney health was monitored by assessment of urea (blood urea nitrogen, BUN) and creatinine concentration throughout the study. Both blood urea concertation (BUN) and creatinine levels remained stable and within the expected range after a single 1 mg/kg dose of ETX024 (FIGS. 38 and 39).

### Example 9: Alternative Synthesis Route for the Conjugate Building Block TriGalNAc_Tether2

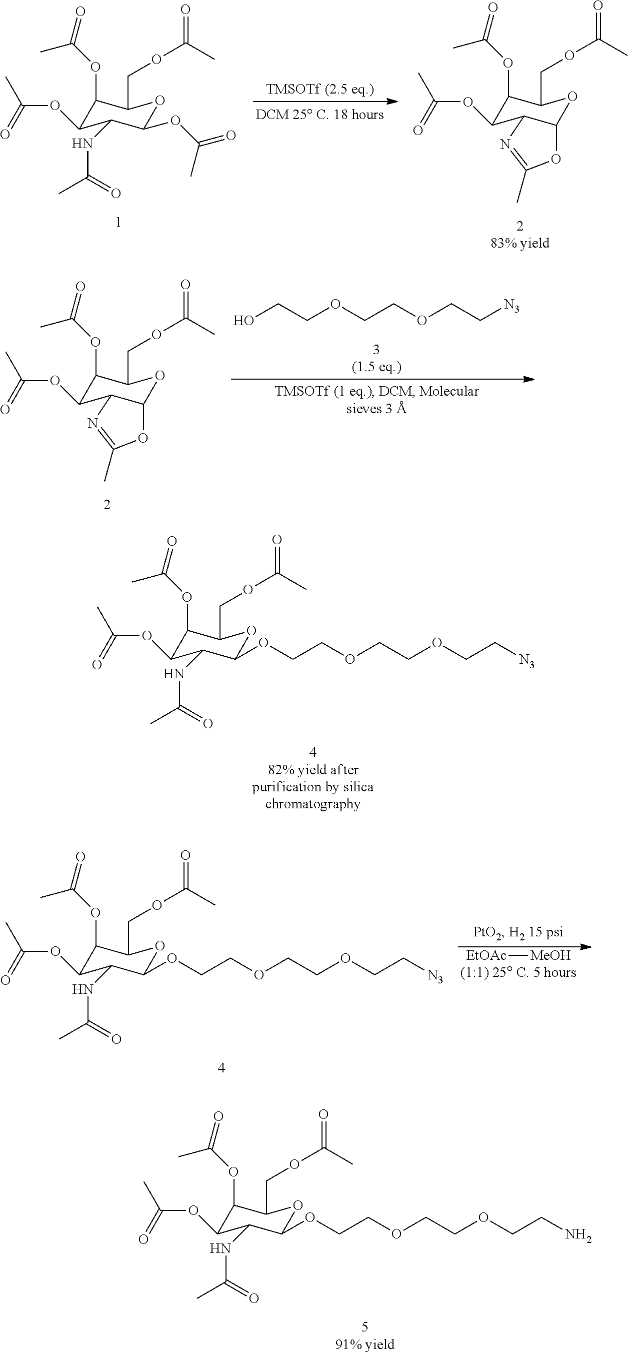

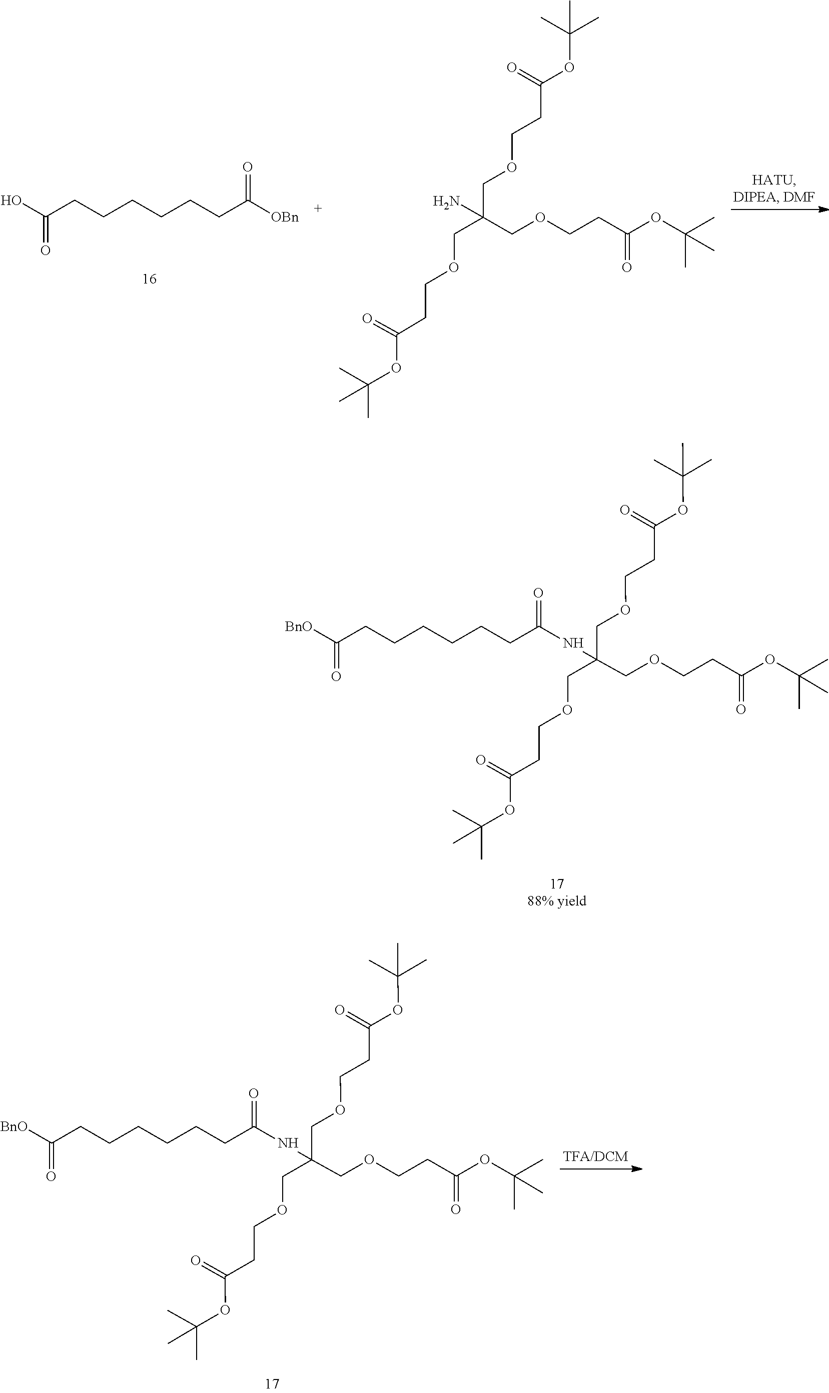 [SMILES: O=C(O)CCCCCCC(=O)OCc1ccccc1 ; CC(C)(C)OC(=O)CCOCC(N)(COCCC(=O)OC(C)(C)C)COCCC(=O)OC(C)(C)C ; CC(C)(C)OC(=O)CCOCC(COCCC(=O)OC(C)(C)C)(COCCC(=O)OC(C)(C)C)NC(=O)CCCCCCC(=O)OCc1ccccc1 ; CC(C)(C)OC(=O)CCOCC(COCCC(=O)OC(C)(C)C)(COCCC(=O)OC(C)(C)C)NC(=O)CCCCCCC(=O)OCc1ccccc1]

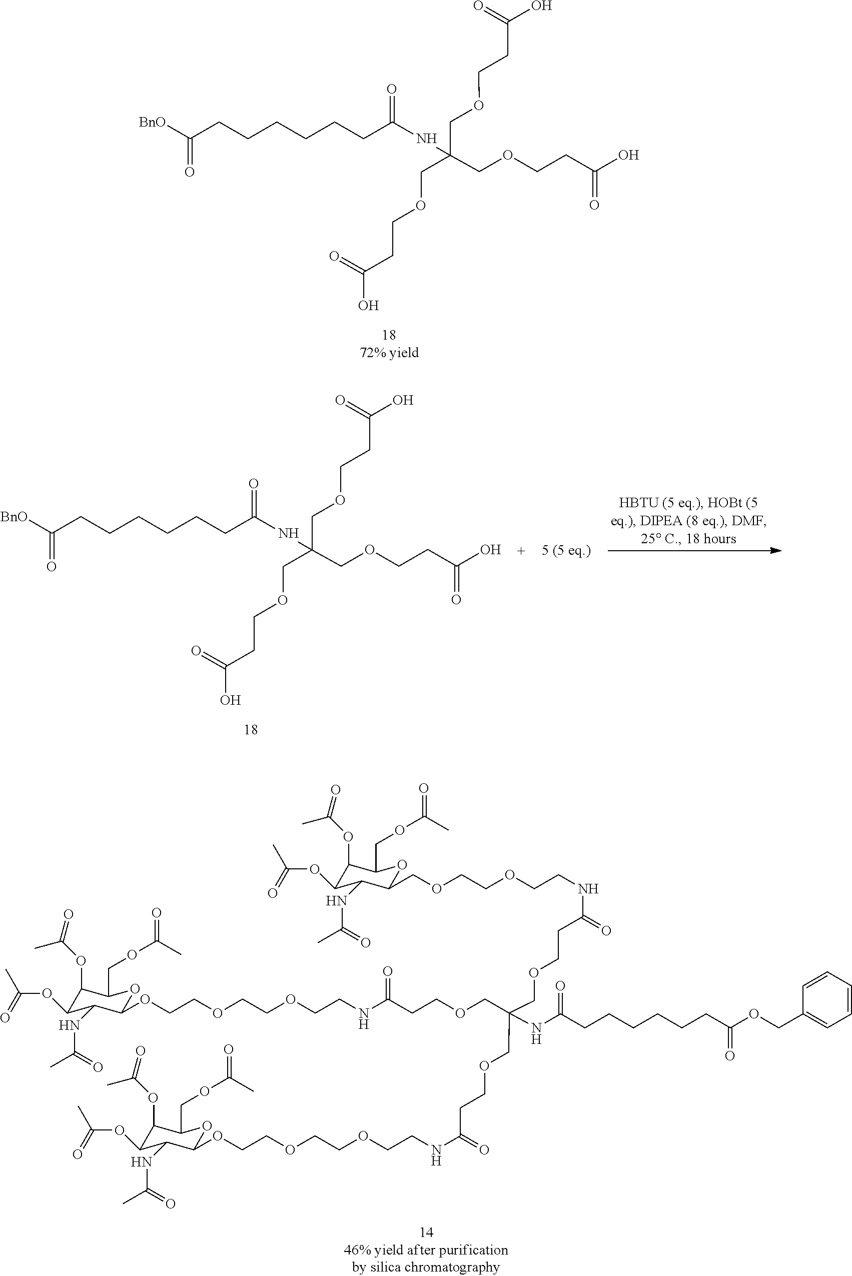 [SMILES: O=C(O)CCOCC(COCCC(=O)O)(COCCC(=O)O)NC(=O)CCCCCCC(=O)OCc1ccccc1 ; O=C(O)CCOCC(COCCC(=O)O)(COCCC(=O)O)NC(=O)CCCCCCC(=O)OCc1ccccc1 ; CC(=O)NC1C(COCCOCCNC(=O)CCOCC(COCCC(=O)NCCOCCOCCOC2OC(COC(C)=O)C(OC(C)=O)C(OC(C)=O)C2NC(C)=O)(COCCC(=O)NCCOCCOCCOC2OC(COC(C)=O)C(OC(C)=O)C(OC(C)=O)C2NC(C)=O)NC(=O)CCCCCCC(=O)OCc2ccccc2)OC(COC(C)=O)C(OC(C)=O)C1OC(C)=O]

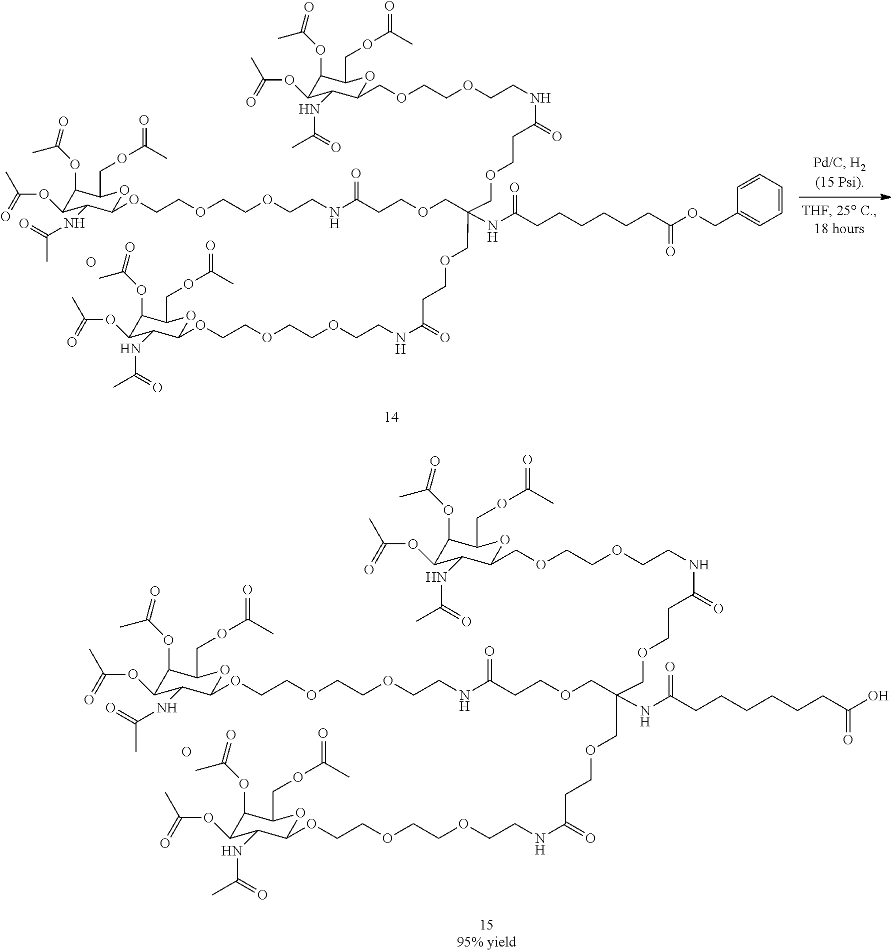

Conjugation of Tether 2 to a siRNA Strand: TriGalNAc Tether 2 (GalNAc-T2) Conjugation at 5′-End or 3′-End

### Conjugation Conditions

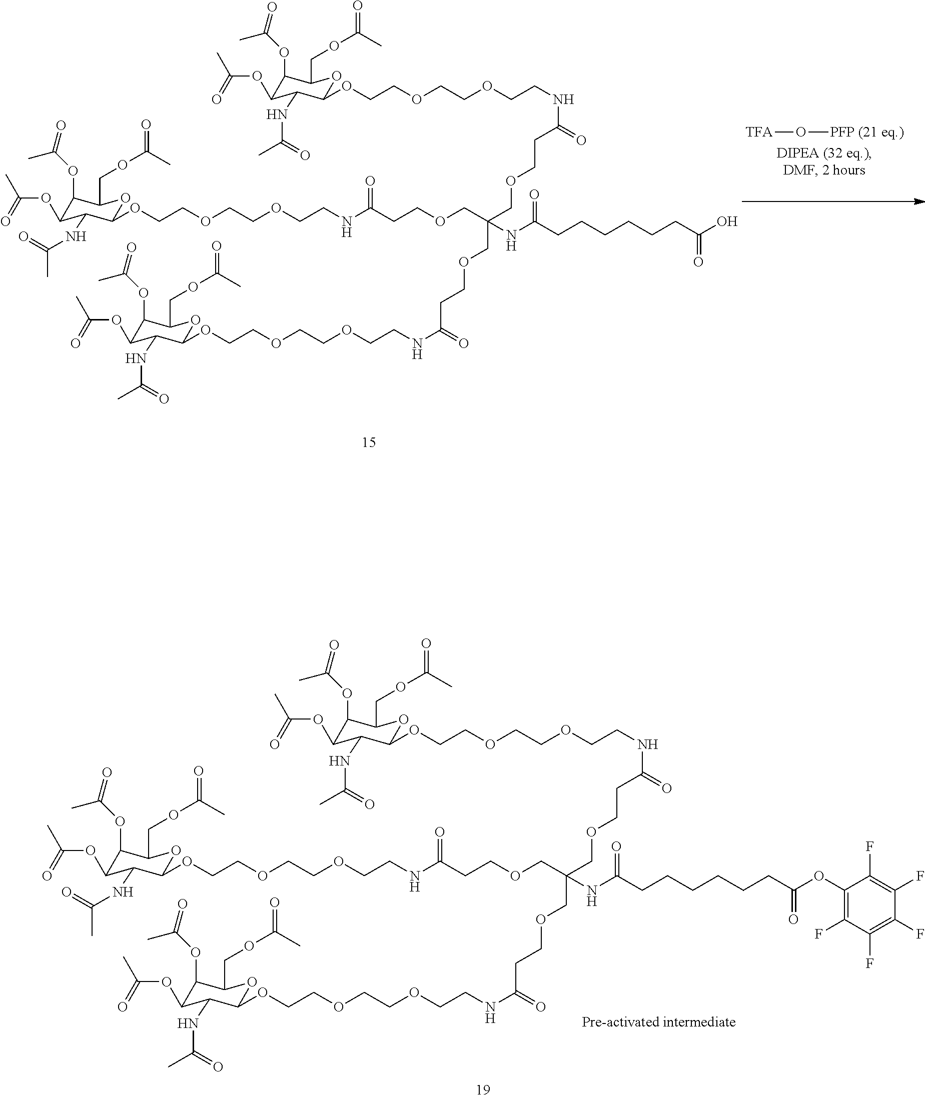

Pre-activation: To a solution of compound 15 (16 umol, 4 eq.) in DMF (160 μL) was added TFA-O—PFP (15 μl, 21 eq.) followed by DIPEA (23 μl, 32 eq.) at 25° C. The tube was shaken for 2 h at 25° C. The reaction was quenched with H₂O (10 μL).

Coupling: The resulting mixture was diluted with DMF (400 μl), followed by addition of oligo-amine solution (4.0 μmol in 10×PBS, pH 7.4, 500 μL; final oligo concentration in organic and aqueous solution: 4 μmol/ml=4 mM). The tube was shaken at 25° C. for 16 h and the reaction was analysed by LCMS. The resulting mixture was treated with 28% NH₄OH (4.5 ml) and shaken for 2 h at 25° C. The mixture was analysed by LCMS, concentrated, and purified by IP—RP HPLC to produce the oligonucleotides conjugated to tether 2 GalNAc.

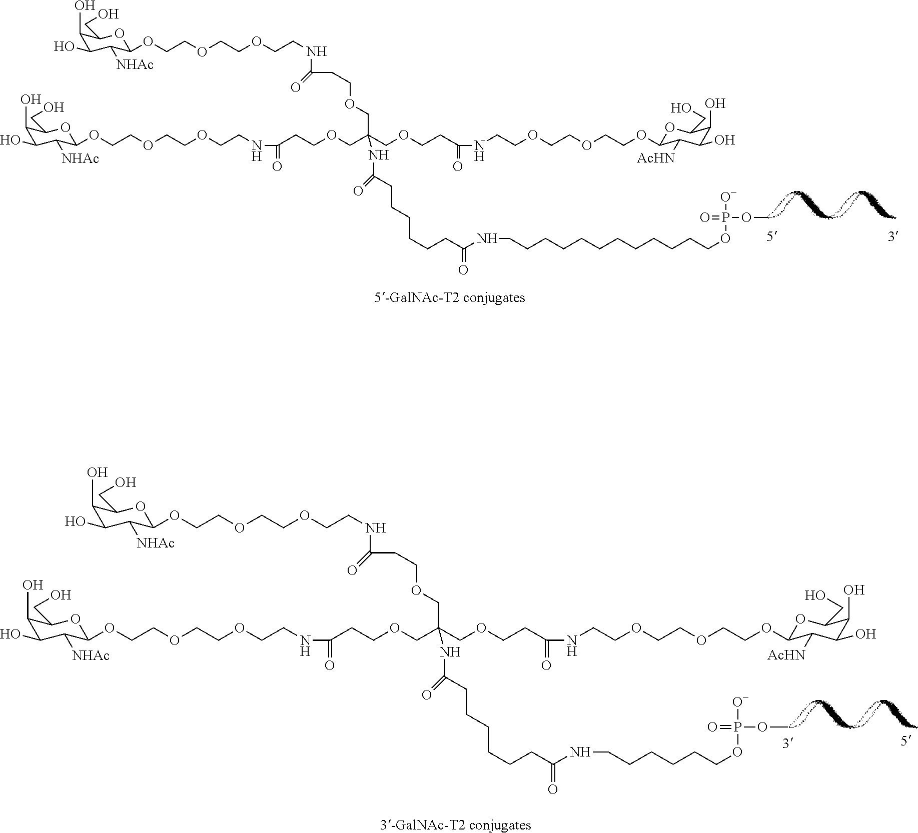 [SMILES: COP(=O)([O-])OCCCCCCCCCCCCNC(=O)CCCCCCC(=O)NC(COCCC(=O)NCCOCCOCCOC1OC(CO)C(O)C(O)C1NC(C)=O)(COCCC(=O)NCCOCCOCCOC1OC(CO)C(O)C(O)C1NC(C)=O)COCCC(=O)NCCOCCOCCOC1OC(CO)C(O)C(O)C1NC(C)=O ; COP(=O)([O-])OCCCCCCNC(=O)CCCCCCC(=O)NC(COCCC(=O)NCCOCCOCCOC1OC(CO)C(O)C(O)C1NC(C)=O)(COCCC(=O)NCCOCCOCCOC1OC(CO)C(O)C(O)C1NC(C)=O)COCCC(=O)NCCOCCOCCOC1OC(CO)C(O)C(O)C1NC(C)=O]

### Example 10: Solid Phase Synthesis Method: Scale ≤1 μmol

Syntheses of siRNA sense and antisense strands were performed on a MerMadel92X synthesiser with commercially available solid supports made of controlled pore glass with universal linker (Universal CPG, with a loading of 40 μmol/g; LGC Biosearch or Glen Research).

RNA phosphoramidites were purchased from ChemGenes or Hongene.

The 2′-O-Methyl phosphoramidites used were the following: 5′-(4,4′-dimethoxytrityl)-N-benzoyl-adenosine 2′-O-methyl-3′-[(2-cyanoethyl)-(N,N-diisopropyl)]-phosphoramidite, 5′-(4,4′-dimethoxytrityl)-N-acetyl-cytidine 2′-O-methyl-3′-[12-cyanoethyl)-(N,N-diisopropyl)]-phosphoramidite, 5′-(4,4′-dimethoxytrityl)-N-isobutyryl-guanosine 2′-O-methyl-3′-[(2-cyanoethyl)-(N,N-diisopropyl)]-phosphoramidite, 5′-(4,4′-dimethoxytrityl)-uridine 2′-O-methyl-3′-[(2-cyanoethyl)-(N,N-diisopropyl)]-phosphoramidite.

The 2′-F phosphoramidites used were the following: 5′-dimethoxytrityl-N-benzoyl-deoxyadenosine 2′-fluoro-3′-[(2-cyanoethyl)-(N,N-diisopropyl)]-phosphoramidite, 5′-dimethoxytrityl-N-acetyl-deoxycytidine 2′-fluoro-3′-[(2-cyanoethyl)-(N,N-diisopropyl)]-phosphoramidite, 5′-dimethoxytrityl-N-isobutyryl-deoxyguanosine 2′-fluoro-3′-[(2-cyanoethyl)-(N,N-diisopropyl)]-phosphoramidite and 5′-dimethoxytrityl-deoxyuridine 2′-fluoro-3′-[(2-cyanoethyl)-(N,N-diisopropyl)]-phosphoramidite.

All phosphoramidites were dissolved in anhydrous acetonitrile (Honeywell Research Chemicals) at a concentration of 0.05M, except 2′-O-methyl-uridine phosphoramidite which was dissolved in DMF/MeCN (1:4, v/v). Iodine at 0.02M in acetonitrile/Pyridine/H₂O (DNAchem) was used as oxidizing reagent. Thiolation for phosphorothioate linkages was performed with 0.2 M PADS (TCI) in acetonitrile/pyridine 1:1 v/v. 5-Ethyl thiotetrazole (ETT), 0.25M mM in acetonitrile was used as activator solution.

Inverted abasic phosphoramidite, 3-O-Dimethoxytrityl-2-deoxyribose-5-[(2-cyanoethyl)-(N, N-diisopropyl)]-phosphoramidite were purchased from Chemgenes (ANP-1422) or Hongene (OP-040).

At each cycle, the DMT was removed by deblock solution, 3% TCA in DCM (DNAchem).

The coupling time was 180 seconds. The oxidizer contact time was set to 80 seconds and thiolation time was 2*100 seconds.

At the end of the synthesis, the oligonucleotides were cleaved from the solid support using a NH₄OH:EtOH solution 4:1 (v/v) for 20 hours at 45° C. (TCI). The solid support was then filtered off, the filter was thoroughly washed with H₂O and the volume of the combined solution was reduced by evaporation under reduced pressure.

Oligonucleotide were treated to form the sodium salt by ultracentrifugation using Amicon Ultra-2 Centrifugal Filter Unit; PBS buffer (10×, Teknova, pH 7.4, Sterile) or by EtOH precipitation from 1M sodium acetate.

The single strands identity were assessed by MS ESI- and then, were annealed in water to form the final duplex siRNA and duplex purity were assessed by size exclusion chromatography.

### Example 11: Solid Phase Synthesis Method: Scale ≥5 μMol

Syntheses of siRNA sense and antisense strands were performed on a MerMadel2 synthesiser with commercially available solid supports made of controlled pore glass with universal linker (Universal CPG, with a loading of 40 gmol/g; LGC Biosearch or Glen Research) at 5 μmol scale. Sense strand destined to 3′ conjugation were sytnthesised at 12 μmol on 3′-PT-Amino-Modifier C6 CPG 500 Å solid support with a loading of 86 μmol/g (LGC).

RNA phosphoramidites were purchased from ChemGenes or Hongene.

The 2′-O-Methyl phosphoramidites used were the following: 5′-(4,4′-dimethoxytrityl)-N-benzoyl-adenosine 2′-O-methyl-3′-[(2-cyanoethyl)-(N,N-diisopropyl)]-phosphoramidite, 5′-(4,4′-dimethoxytrityl)-N-acetyl-cytidine 2′-O-methyl-3′-[(2-cyanoethyl)-(N,N-diisopropyl)]-phosphoramidite, 5′-(4,4′-dimethoxytrityl)-N-isobutyryl-guanosine 2′-O-methyl-3′-[(2-cyanoethyl)-(N,N-diisopropyl)]-phosphoramidite, 5′-(4,4′-dimethoxytrityl)-uridine 2′-O-methyl-3′-[(2-cyanoethyl)-(N,N-diisopropyl)]-phosphoramidite.

The 2′-F phosphoramidites used were the following: 5′-dimethoxytrityl-N-benzoyl-deoxyadenosine 2′-fluoro-3′-[(2-cyanoethyl)-(N,N-diisopropyl)]-phosphoramidite, 5′-dimethoxytrityl-N-acetyl-deoxycytidine 2′-fluoro-3′-[(2-cyanoethyl)-(N,N-diisopropyl)]-phosphoramidite, 5′-dimethoxytrityl-N-isobutyryl-deoxyguanosine 2′-fluoro-3′-[(2-cyanoethyl)-(N,N-diisopropyl)]-phosphoramidite and 5′-dimethoxytrityl-deoxyuridine 2′-fluoro-3′-[(2-cyanoethyl)-(N,N-diisopropyl)]-phosphoramidite.

Inverted abasic phosphoramidite, 3-O-Dimethoxytrityl-2-deoxyribose-5-[(2-cyanoethyl)-(N, N-diisopropyl)]-phosphoramidite were purchased from Chemgenes (ANP-1422) or Hongene (OP-040).

All phosphoramidites were dissolved in anhydrous acetonitrile (Honeywell Research Chemicals) at a concentration of 0.05M, except 2′-O-methyl-uridine phosphoramidite which was dissolved in DMF/MeCN (1:4, v/v). Iodine at 0.02M in acetonitrile/Pyridine/H₂O (DNAchem) was used as oxidizing reagent. Thiolation for phosphorothioate linkages was performed with 0.2 M PADS (TCI) in acetonitrile/pyridine 1:1 v/v. 5-Ethyl thiotetrazole (ETT), 0.25M mM in acetonitrile was used as activator solution.

At each cycle, the DMT was removed by deblock solution, 3% TCA in DCM (DNAchem).

For strands synthesised on universal CPG the coupling was performed with 8 eq. of amidite for 130 seconds. The oxidation time was 47 seconds, the thiolation time was 210 seconds.

For strands synthesised on 3′-PT-Amino-Modifier C6 CPG the coupling was performed with 8 eq. of amidite for 2*150 seconds. The oxidation time was 47 seconds, the thiolation time was 250 seconds

At the end of the synthesis, the oligonucleotides were cleaved from the solid support using a NH₄OH:EtOH solution 4:1 (v/v) for 20 hours at 45° C. (TCI). The solid support was then filtered off, the filter was thoroughly washed with H₂O and the volume of the combined solution was reduced by evaporation under reduced pressure.

Oligonucleotide were treated to form the sodium salt by EtOH precipitation from 1M sodium acetate.

The single strand oligonucleotides were purified by IP—RP HPLC on Xbridge BEH C18 5 μm, 130 Å, 19×150 mm (Waters) column with an increasing gradient of B in A. Mobile phase A: 240 mM HFIP, 7 mM TEA and 5% methanol in water; mobile phase B: 240 mM HFIP, 7 mM TEA in methanol.

The single strands purity and identity were assessed by UPLC/MS ESI− on Xbridge BEH C18 2.5 μm, 3×50 mm (Waters) column with an increasing gradient of B in A. Mobile phase A: 100 mM HFIP, 5 mM TEA in water; mobile phase B: 20% mobile phase A: 80% Acetonitrile (v/v).

Sense strand were conjugated as per protocols provided in any of examples herein.

Sense and Antisense strands were then annealed in water to form the final duplex siRNA and duplex purity were assessed by size exclusion chromatography.

### Example 12: Nucleic Acid Sequences

siRNA oligonucleosides suitable for use according to the present invention can target HCII and ZPI. The full DNA sequences of the HCII and ZPI targets are respectively as follows (SEQ ID NOs: 1 and 2):

(HCII)
SEQ ID NO: 1
TTGCGCTTCTAGAATGCTTCCCTCTCAATGAGAACAGTAGCTCCACGTGGCTGGGAAGTT
CAAAGTGGTTTTGACACAGAAAAGAGGAAGTAAGTGGACTCTATCTTTGATTTGGGATC
CTACTCCTGACCCTGTGAACTTCTTGGCTCCCTCTTGAGGACGTTGGCTTGAAAGTGGCT
CTGTGGGTTCTCCCTGCTCTCTGACTTCTCCGAGCCTGCTGGCCACTGTCTTGGCTGAGAC
TGCTCTAGTCTCCAGAAAGGAGATCTGCTCACTCCTAAGAAGTATCAAGGTCAGGCCAG
GTGTGGTGGCTCACGCCTGTAATCCCAGCACTTTGGGAGGCCAAGACAGGCAGATCAGG
AGGTCAGGAGATCGAGATCAGCCTGGCTAACACGGTAAAACCCCATCTCTACTAAAAAT
ACAAAAAATTAGCCAGGCGTGGTGGCACACACCTGTAGTCCCAGGTACTCGGGAGGCTG
AAGCAGGAGAATCGCTTGAAACCAGGAGGCCGAGGTTGCAGTGAGCCAAGATTGCGCC
ACTGCACTGCAGCCTGGGCGACAGAGCGAGACGCCATTTCAAAAAAAAAAAAAAATCA
AGGTCAGGGGGGAAGTGGGAAGACTGAAATAGATAAAGGATTCTAAAGAGATATAACA
GTCAAATGCGACACATGAAACCCTGACCAGATAAAAATTAAAAACCCATAAAATACATG
TTTGAAGTCATAGAGTAATCTGACTTGGACTAGACATGTGATATATGTGAGGCTTGTGAT
CTTCCCAGGAGTGATGGTAGCACAGCACAGGGCAGAGACCCGTCCATGGAAGAAACACT
GGTGCTAGTGCCCAGGGCAGAAGTGAGTGATGTCTTTAAGTGGATATGGAAAAATATTA
ACTATTCTACCTAGGTTGTGGGTGTATGGATATTTAGTATTCAATTATTCCAATTTCTCTG
TGTATGTATACATATTTTTTTTAGAGACAGGGTCTCACTCTGTCGACCACACTGGAGTAG
GGGGTACAATCATAGCTCACTGTACATACTCAAGTGATCCTTCTGCCTCAGCCTCCTGAG
CAGATGGGACTACAGGTGTGCAGCATCATGGCCCAGTTTTTTTTTTTTTGGTAGAGATGG
GTTTTGCTAGCCGGGAGCAGTGGCTCATGCCTGTAATCCTAGCACTTTGGGAGGCTGAGG
CGGGCAGATCATCTGAGGTCAGGAGTTCAAGACCAGCCTGGGCAACATGGTAAAACCCT
GTCTCTACTAAAAACACAAAAATTAGCCAGGCATGATGGCAGGCGCCTGTAATCCCAGC
TACTTGGGAGGCTGAGGCAGGAGAATCGCTTGAACCCAGGAGGCAGAGGTTGCAGCAA
GCTAAGATTGAGCCACTGCACTCCAGCCTGGGCAACAGAGCAAAAACTCCGTCTCAAAA
AAAAAAAAAAAAAAAGAGAGAGAGAGAGATCGGTTTTGCTATGTTGCCCAAGCTGGAC
ACGGACACACACACACACACACACACACACACACACACACACACACACACACACACAA
GCTGGACACAGAGACACACACAGTGACAGGGCAAAGGTTCCAAAATTTTAAACCTGGTA
AATCTGGGTACGGGTATACAGGAGTTGTTCTACTACACTATTCTTTCAACTTTTTTGAAA
GTTTGAAGTTATTTCAAAAGAAAAAGTTTTCCAAACTTTAGTGATCCTCCTGCCTCAGCC
TCCCAAAGTGCTGGGATGATAGGCATGAGCCACCGTGCCTGACCCCTCTGTATATTTTTA
GAATTTCATGTTAAAAGATGGAAAAGTCTGGATGAGGTAGTTCACGCCTGTCTTCCCAGC
TCTTTGGGAGGCCAAGGTGGGAAGACTGCTTGAAGCCAGACGTTCAAGACCAACTTGGC
CAACATAGTGAGACCCCGCTTTTTTCTAACTAAAAAAATTTTTTTCCAAGTTGGAAAAAA
TATCTAGCCATAAGACAAACCTTGAAACTGCAAAAGAACAATGGAGTATGTGTGACAGG
AGGTACTGCTCTACAGTGGGGTTAAAGCCATACACAAGCTGTGGTGGCTCACGCCTGTA
ATCCCAGCACTTTGGGAGGCCGATGCGGGCGGATCATGAGGTTAGGAGTTCAAGACCAG
CCTGGCCAGCATGGTGAAACCCGTCTCTACTAAAAATACAAAACATTAGCCAGACGTGG
TGGTGGGCACCTGTAGTCCCAGCTACTAGGGAGGCTGAGGCAGGAGAATGGCGTGAACC
CAGGAGGCGGAGCTTGCAGTGAGCTGAGATTGCGCCACTGCACTCCAGCCTGGGCGACA
GAGCGAGACTCTGTCTCAAAAAAAAAAAAGCCATACACAAGCTGTTACCACTAAATGGG
AAAATGACTGAAAAATGTCAATGTCAAGAGGGACTGAAATCAAATTTTTCCAATAGTGG
GTTACATGATCAGAAATCCAAATAGACAGGAAATATGTTGGCTTTATTTATTTATTTATT
TATTTATTTATTTATTTAGACAGAGTCTCACTCTGTCACCCAGGCTGGAGTACAGTGGCA
TGAACTCGGCTCACTGCAACCTTCACCTCCCAGGTTCAAGCGATTGTCCTGCCTCAGCCT
CCCGAGTAGCTGGGACTACAGATGTGTGCCACCACACCCAGCTAATTTTTGTATTTTTAG
TAGGGACGGGGTTTTACCATGTTGGTCAGGCTGGTCTTGAACTCCTGACCTCAAGTGATC
CACCCGCCTTGGCCTCTCAAAGTGCTGGGATTACAGGTGTGAGCCACCACACCTGGCCG
GTACTGGCTTTAAAAATAACAAAAGTAATACATACACATAGAAAAAGGTCAAACAAAG
AAGTACATAGAATGAAAAATGAATGCTGTGTCCCCTCCCAGACCATTTCTGTGAATAAA
TATGTAATACCATGAAATGATGAGGACTAACATTTTCTGAATGCCAGGCACCACTCTATG
TGCTTTCCACACATTCATTAACCTCATTTAATTTTCTCATTTAATTAATGAGATAAATTAA
TGTATCTCATTTAATTTTCACAACAACCTCATGCAGTAGGTGTAACTGTCACCCTCATTTC
AGAGAGCAGAATACTGAGAGCTGGAGGCCAAGGGGCAATTTCAGCCAGGGTGGCTGGT
GACGCCTCGGTGAAACCAAGAGCGAACAGTGAGAGCAGCGGCCACCTGCTGGTCTGCA
GGGATGGTGTCCTGGGCAGAAAGAATAGCAAGTGCCAGGGCTGTGCTGGGGCCGGGCTT
TGCATGTGTGAGAACAAGACAGAGAATGAGGGAGGTGGGCCCACGAGGAGTGTGGGCA
CAGACAGCAGCCTCTGCCTGTGGTGCCACGCTGAAGACTCAGTATTGTATGTGACAGAT
GAAGGCTCTAAGAAGACAGCTCTGACAAAAGCTAGAGTGCAAAATCAGACTCAGACAC
AACCACCGGTCTGTGTCCTGAACACAATGGACCTTTACACTCTGGAATTTCTCAAACGGA
GCAATGCACAGACACCCCCATGGGCCCCTTGCACACCCGCAGATTCTCCTAGGAGTCAC
ATTCTCTCTTCAGATAGACTCTGGGTGCCGACACTCCCAAACATGCTCTTGAGGAGCAGT
CTCTGTGATAAGCTGATCTTCCAGACAATCCAGAATATTCTTAAAACTTTTTAGATCATA
AAATTTAAAACACAAATTAAAAAACAAATTATCATAAGGCCGGGCACAGTGACTCATGC
CTGTAATCCCAGCACTTTGCAAGGCTGAAGCAGGAGGATCACTTGAGCCCAAGAGTTCA
AGACCAGCCTAGGCAACATAGTGAGACCCTGTCTCTACAAAAAAGTCAAAAGTTAGCTA
GACATGGTGGTGTGCACCTGTATTCCCAGCTACTTGCAGGGCTGAGGTGAGGAGGATTG
CTTCAGCTCGGGAGGTTGAGGCTGCAGTGAGCCAAGATCACGCCACTGCACTCCAGCCT
GGGTAACAGAGTGAGACCCTGTCTCAAAAAACACATAGGGCCAGGCGTGGTGGCTCACG
CATGTAATCCCAGCACTTTGGGAGGCCGAGACGGGAGGATCACTTCACTCCAGGAGTTC
AACACCAGCCTGGCCAACATAGTGAAACCCCGTCTCTACTAAAAATACAAAAAATTAGT
TGGACATGGTGGTGTGCGCCTGTAATCTCAGCCACTCAGGAGGCTGAGGCAGGAGAACG
CTTGAACTTGGGAGACAGAGGTTGCAGTGAGCTGAGATCGCACCACTGCACTCCAGCAT
GGGCAGCAGCGCGAAACTCTGTCTCAAAACAAACAAACAAACAAACAAACACCCATAA
ACACAAAATGTATCACAGCCTCAGAGATCCCCACGAATGCCTAAGTGGCCCTGAATTTG
GGAGGCACTGCTCAGTAATAGTCCTATCTGTCCCACAACAGACAGGAGTGCTGGGCTGC
ACCTACTGGCAACAAACACAGCAACCCTTGACTGAAGAAAGGTCCATGCCACAATCCCC
TTATTCTGTAAGCCACTAATTTTGTCCTCTCTCCTCCACCTTTCACTGAGGAACGAGCTCT
TGGAAGGACAGGGACACCCGCCTAGTAGCTGAGCCAGCCACATCAGTCCTGGAGAGCA
GGTGGAGGGCAGATGCTGTGATCATCCCAGAAGAGAGGACACAGTTGGAGGCAGATGC
ATGGTCTCTACTTTCAGCTACCCTCAATGCAGCCTGGTCCCCAGAGGCCTGAAGAGCGCC
TTGTTTATGTGGTGACCTCAAGAGGGGCTGCTCCTGCACCAAGGCTATGTGTGCATGCTA
ACACAGTAACCGTCATATACTCAAAGTGTCAGCTCTAAGAACTGGAGATGAGGAGCTGC
AAGCCACTCTACAGTTATCAAAGGCACAGCTGAGGGGGTTTGTGCTGACCAAGCTGGTT
GCCTGGTGTTTGGATTGGGACTTATTTACTTTGGAAAATATGCAGCAACAGCCCAGCACC
AAAGTTCACATCAAAATCCCACTGATGACCTTGGCTGCTTTCATCTCTGAAGCGCCACTT
CTCAGAAACACAGAGGTAAGTTGGGTTTCTAATGTTTCTGCTGATTATAAATTATTTTTG
GTGTTTACGGATAGGCAACTGGTTCATTTTTCTAGCAAACTAAGAATTCAGAAGCTTTCT
ACACTGTTTTAGAAGTGGGAAATGGTTTCATTTTTCAGTGTGCCTATTATAAAATTGTGT
CAGTTCCATTGTTGGGAGAGTTGACAAACTTAGAATAGGAGCTGTGGAATAGATGAAAA
TATTGTACTTATATTAAATTAATCGAATTGGATAACTGTCCTGTGATTATGTATGAGAAT
ATCCTTGCTCTTGGGTATTTTCCCTGAAGTATTAGTATTAAAGGTTAGAGGGGCCGGGTG
CAGTGGCTCACGCCTGTAATCCCAACACTTTGGGAGGCCGAGGCGGGTGGATCACGAGG
TCAGGAGTTCAAGACCAGCCTGACCAACATGGTGAAGCCAAGTCTCTACTAAAAATACA
AAAATTAGCTGGGCGTGGTGGCACGCGCCTGTAATCCCAGCTACTCAGGAGGCTGAGGC
AGGAGAATCGCTTAAACCCGGGAGGCAGAGGTTGCAGTGAGCGGAGATCGTGCCACTG
CACTCCAGCCTGGACAACAGAGTTAGACTCCGTCAAAAAAAAAAAAAAAAAGAAGAAA
AAAGAAAAAATGTTAGAGGAACAAGATATAGGAGACCTACTCTCAAATGGTCTAGAAG
AAAAAATGTGTATGTGCATGCCTGTGAGAACACACACGTACGTACACACACACACAGAT
AATGACAGGGCAAAGGTTCCAAAATTTTAAACCTGGTAAATCTCGGTACGGGTATACAG
GAGTTGTTCTACTACACTATTCTTTCAACATTTTTGGAAGTTTGAACTTACTTCAAAATAA
AAAGTTTTCCAAACTTTAGGCAGTTACTTCTCTCCCATTCTGCCTGCTCTGTTGGGCCTGG
AGACCATACACCAGGAGGGATGACGGTTTATCAAGTGTTATGCTCTGATGCGTGACTGA
AAAGGCCAACCCAGCTCTGGCAATTAGCAAGAAAGCACAATATGAAGTTCCCAGGAAA
AAAAAAAAGCAAAACAAACTTTTGAATGATTTATCTTTAAAATATATTGTTTCTCTTCAA
ACAGTAATCTGGATTTAATCACAACCTAGTGATAGTTTTTAAACGTCTTCTACAATGTTT
GTTATACTAAATAGCAAAACATCAGGAAGATTTACCTTCAGATCTTTAATTTCAATCCAT
AAAAGATATCAGAGATATTTTCTCCTTCCTCTGGTAAGGGAATGACGAAAACTATTTTTG
GCTTTTTATCAGATAATGTGGGAACAGGGTATAAGAAGTTTCCAAATATAACTTCTGAAT
ACCGGGATAAAACATGCATGTCTTTACTCTGCCACTCTATCTGGCCTCAGATACGTTTTC
CTGAATGCTTATTTATTCAAGTTGGTTTTTGTTTTGTTCTTTAACCTTATTTTTATCTGAGA
AGAAAACATTTTCCCCCTTTGTTCCTTCTTCTTTTGGCTTTCTTTTTTAAAATAGAGATGA
GGTCTTGCTATGTTGCTCCAGCTGGTCTTGAACTCCTGGGCTCAAGCGATCCTCCTGCCTT
GGCCTCCCAAGATGCTAAGATTACAGGTGTGAGCCCCTATGCCTGGTCTTCTTCTTCTTG
ATCTTAGCCAAAAGGCCAAGAAGTGATAAGAGGAGGACACTTGAAGTGTAGTTGGGCA
AGGAGCCTTCTACCAGCTGCTTACTTTCTTTGTTCCTGACTTTTAAAAGTGTGTTGCTATT
GATACACAGTCTCCTGATATGTAAAATGCTGGGAGGATGAAGCTAAGTTACTCAAAGTG
CCATTCAGAAACTGGGCCCAGTTCTATTTGCAGCTACATACATTAGAAATCATTTCTAGA
GGCTGAGCATGGTAACTCATACCTGTAATTCCAGCACTTTGGGAGGCCAAGGCAGGAGA
ATTGCCTGAGCTCAGGAGTTTGAGACCTGTCTGGGCAACATGGTAAAACCCCATCTTTAC
CAAAAACACAAAAAATTAACTGGGTTTGGTGGCACACACCTGTGGTCCCAGCTACTTCA
AAAGGCTGAGGTGGGAGGGTCTCTTGAGCCTGAGAGGAACAGGTGGCAGTGAACCAAT
ATTGTGCCACTGCACTCCAGCCTGGGTGACAGAGTGAGACCCCGCCGTCTCAAAATAAA
AATAAAAAGAAATCGTTTCTAGAAACTGTTTTCCCGTGTGTAAACTAGTGGCACTGCAGC
CTGAGGCAGGTGCTGAGATGGGGACCTGGAAAAGGCAACAGGCATTTTGAGTCAGAAA
CAATGTGACTTTCCTGCTCCAAAATGTGCAATTCAAAAGTCTTTCTTAGTTGTGACTAAA
ACAAACTTTGAACTTACTATTTCAACAGTATTATAAGGGGAAGACCCAAGGAATGGGAC
TGGCACTGGGAAAACAGCTAGGAAGCTGCTCTGCACGGCCAGGGAGTCTGGAAGCATCC
TGGTACTCCAGAGCGAACAAGGCTGAGCGCTTGATGTGGGGCTTAGAGGCTTAACCAAC
TTGGTTCGAATCTAGCCACTGCCACTTATTAGTGACAGTGACGAAAGGCTCAGTCTCCTG
ATATATAAAATGTTGGGAGGATGAAACTAAGTTACACGAAGTGCCTTATACAGCGTGTC
AGGCATCCAACAGAGGCCATTATCAACATTAACCACACTGACAGCATTTCAAGCAGAGT
ATCCGAACAGTTACCCCATCTTCAGGCCTACTGAGTTCAAATATTTGCTTAACAAGAGCA
GCCAGTAACTCTTACCTGGCCTCAACTGGCAGCAGATATTCTGGGCCTCAAATATCTATC
TAATAGGAAATGGTCACAGACACAAAATAAGCTTAACAAAAGGCAGTTTTTTTTTGTTTT
TTTTTTGTTTTCTGTTTTTTGAGATAAGGACTCACTCTATCCCCCAGGTTGGAGTGCAGTA
GTGGCGTGATCACGGCTCACTGCAGACTCAAGTGATCCTCCTACTTCAGCCTCTCAAGTA
GATGGGACCACAGGCGTGTGCCATCACACCAGGCTAATTATTTTTCTTTTCTTTTTTTTTT
TTTTGAGACGGAGTTTCGCTCTTTTTGCCCAGGCTGGAGTGCAATGGTGCGATCTTGGCT
CACCACAACCTCTGCCTCCTGAATTCAAACGAATCTCCTGCCTCAGCCTCCTAAGTATCT
GGGATTACAGGCATGCGCCACCACGCCGGCTAATTTTTTTGTATTTTTTGTAGAGACAGG
GTTTCTCCATGTTGGCCAGGCTGGTCTCGAACTCCCGACCTCAGATGATCCGCCCACCTC
GGCCTCCCAAAGTGCTGGGATTACTGACCTGAGCCACCGCACCCAGCCTATTTATTTAAT
TTTTCACAGAGATGAGGTCTTGCTATGTTGCCCACACTGGTCTTGAGCTCCTGGGCTCAA
GTGATCTTCCTGCCTTGGTCTCCCAGTGTTGGGATTATAGGCGTAAGCCACAGCGCCTGG
CCGGCAGTTCTTTCTGGGGTGATTAGAAGTTGGGACCATGTATTACCTGTCTGAGTCAGC
ATTATAAACACCTATGGTCACTGTCCTGGCAAAACATGGAATCATCAAAGCTCATCTAAC
CAGAGTGCAGTTAATAACCAGGAAGTAAGCAAGAGAAAGACAAAGGATTTGGCAGTCA
AAACAGATTTGACAGGCCAAGTCAGATCCTCCTCTGAACGAGTCAGAGGAACAAATAAA
GACAGGATTGCCATAATGCCTCTGTGCTAAAAGCTTATCTTGTTTACTTAAATAAAGGGA
GTGCCCCTCAGGTCTTGAGTAAGAGCTTGCTGACATCACCCTCACACAGACTTTATCTCT
TGTTTCTAACCCTGTGTTAGAAGCAGTAACACAGAAGATTTAGTTGCTCCTGACAGCAGT
GGGAGCTATTGTCTAAGAGATACAAAGGAGAAAAAAGTATACCTGCAGCAAGTGATATC
ACCTCTGGGGCTGCCACCACATCACCTCACTACGCCCTGAGGGGGTCTCAGCACTAGAC
AAGTTCCAAATCTTTTGCAAATTAAACAACCCCAGGTCAGGCGTGGTGGCTTATGCCTGT
AATCCCAGCACTTTGGGGGGCTGAGGTGGGTGGATCACCTGAGGTCAGGAGTTTGAGAC
CAGCCTGGCCAACAGAGCAAAACCCCATCTCTACTAAACAAAATACAAAAATTAACCAG
GCGTAGTGGTGTGCACCTGTAGTCCCAGCTACTTGGGAGGCTGAGGCAAGAGAATTGCT
TGAGTCCAGGAGGCCGAAGTTGCAGTAAGCCGAGATCGCGCCACTGCACTCCAGCCTGG
GTGACAGAGTGAGACTCCATTTCAAAAAATAAAAACAACAAAAGCCAATTACAACAAC
AACAACAAAAAAACAACGAATTAAACAACCCCAAAGATTGCACAAATTTCAAGTATCTT
TAGAATATGTTTTCAGAAAGCCTGGCCCATGGACATTTTTCAACAGCATCTCCATTGCAA
AGGTGGAATGGTGTGAGTCACACAGGCATGGCTGAGTCCCACTAATGCACATCCCTTCT
AGGTACTCTCCAATCACCAGCCCCAGGTGCCCACTCAAGCCCAGCTCTTAGTGAGGTTTC
CCTGACTCTCTGGGCACTTCCACTCCTACCACACAGGGTAGAGCCACACCCCTTTCCGTA
CCCCCATGTGCTCTGGCAGCATTATTTTGAGAGCCTTCGCTTTACTGCACGTCTGTCCCAT
CTGTCCCCTGACTGGTCCATGAGCCCCTGGTGGGAACTTTGTCTCTGGTAACTAAACACT
GTCTGGAGGTGGTGGACAAGGTGTCTGGAGAAAAACAAACTCCTCCCTGGGATGCCTGA
GCTCCCAGGATTCTAGAAGGTTAGTTTTGCAAACCTTTAAAGAAGGGATTTTCATCAAGG
GGCCCACAGATCCTTCATTGAGGTTTATGAGTCCCACATCAAAGGTTGGGTGTCTATCTA
CATCAGATTCTCTTAAAGTCCATGATCCTAAAACAGTTAAGAACTAATGCTGTGAGGGCC
TCTTCCTGGGTCAAAGCCACAGGGAACCTGCCATGTGGATGCTGCAGCGGGGTGTGGAT
CAGCCAGGCCGCCTTTCACTGTGTTCTGTTTTCCCTCCCAGCTTTAGCTCCGCCAAAATGA
AACACTCATTAAACGCACTTCTCATTTTCCTCATCATAACATCTGCGTGGGGTGGGAGCA
AAGGCCCGCTGGATCAGCTAGAGAAAGGAGGGGAAACTGCTCAGTCTGCAGATCCCCA
GTGGGAGCAGTTAAATAACAAAAACCTGAGCATGCCTCTTCTCCCTGCCGACTTCCACA
AGGAAAACACCGTCACCAACGACTGGATTCCAGAGGGGGAGGAGGACGACGACTATCT
GGACCTGGAGAAGATATTCAGTGAAGACGACGACTACATCGACATCGTCGACAGTCTGT
CAGTTTCCCCGACAGACTCTGATGTGAGTGCTGGGAACATCCTCCAGCTTTTTCATGGCA
AGAGCCGGATCCAGCGTCTTAACATCCTCAACGCCAAGTTCGCTTTCAACCTCTACCGAG
TGCTGAAAGACCAGGTCAACACTTTCGATAACATCTTCATAGCACCCGTTGGCATTTCTA
CTGCGATGGGTATGATTTCCTTAGGTCTGAAGGGAGAGACCCATGAACAAGTGCACTCG
ATTTTGCATTTTAAAGACTTTGTTAATGCCAGCAGCAAGTATGAAATCACGACCATTCAT
AATCTCTTCCGTAAGCTGACTCATCGCCTCTTCAGGAGGAATTTTGGGTACACACTGCGG
TCAGTCAATGACCTTTATATCCAGAAGCAGTTTCCAATCCTGCTTGACTTCAAAACTAAA
GTAAGAGAGTATTACTTTGCTGAGGCCCAGATAGCTGACTTCTCAGACCCTGCCTTCATA
TCAAAAACCAACAACCACATCATGAAGCTCACCAAGGGCCTCATAAAAGATGCTCTGGA
GAATATAGACCCTGCTACCCAGATGATGATTCTCAACTGCATCTACTTCAAAGGTAAGA
GGCACCTTTACAGTTCTCACAGCAAACCCACAACATACTATTTTTGTATGTGGGTAGATT
GAATGCCAAGAACTGTACTGTAGCTATAATTTATCCAGGAAAACTAGACACAAGATTGA
CTCTGGAACGGGGACAGGGAAGGCCAAGCTGAAGTGACAGTAGCATCTGACACTTACTG
AGCCCTAACTCTGTGCTTTAACACAGCCTTGTGAGGTCATCACTGTTATTAGCATCCCCA
TTTTACAGAGGAAGCCACCAACACATGAAGTAAAAGGATGGGCTGGGCGCGGTGGCTCA
CGCCTGTAATCCCAGCACTTTGGGAGGCCGAGGCAGGCAGATCACTTGAGGTCAGGAGT
TCGAGATCAGCCTGACCAACAGACCAACATGGTGAAAACCTGGCTCTACTAAAAATACA
AAAATTAGCTGGGCCTGGCGGTGGGTGCCTGTACTCCCAGCTACTTGGGAGGCTGAGGC
AGGAGAATCACTTGAACCTGGAAGGCAGAGATTGCAGTGAGCCGAGACTGTGCCACTGC
ACTCTAGCCTGGACGACAGAGTGAGACTCCATCTCAAAAAAAAAAAAAAAAGAAGTAA
AACGATGCTCCAAGGGCACCCAGTTATTAAGGGGCAGAGCCAAAGCTGAACCCAGGGA
GGCCAACCCTAGCAATCTGTTAAATTGGAAGAAATAATACAAAAACTGTTTTAGCATTT
GGCCAGCCTGGATTTGAGTTTTCTCTTTTCCTTTCCCAATTATCAATAAGCAGGAATATA
GACAAAAGGCTAAAGAAATGCACCTGTGAACTATTCAGCTTGAGCAGCTGACATTGACA
CCTACAAGTGCTTTTCAGGATACTTTTGAACTACTGGGCAGGTGGGATGGAGAAATAAA
TTACTATTTCCCCAGCAACTGTTCTGGGCTGAGCACAAGGGCACTTTTTAAGGAGGTCAC
CCCACACCCATCACACACACATAGGACCCCTGGAATCCTAGGAATAAATAAGCATGGAT
TTGTAAAATCCAAACCTCTCTTTTCAAATATCCTCACCTGGACCAGACCAGAAGAAACCT
CTACTTTACTCTCTAAGCTGAGAGTGTGGAAGGGGAAACACGAGGAATGGTTCGGCTTC
AGGACTAATTGCGGTGACACACAACCACTTCTCTTTGCCACCAAGGACTACCAGGTACCT
GCAAAGGGCAGTACTTGGAGGCCAGTGCTTTCTGCTAGTTAGCTCCCGTGGTTTTATAGC
AGCCCAGGCGAAGGAAGGAGACCCCCCCCAGCTCCTGGCTTCTGTTCAGGGAAAGGGGG
CCAGAGCCCCTCCTGATCTGTCCACACACCTGCTCTGTGCCTTGGCTGAGGCCCCTGCAG
CTCTACAAGGCAGGCATTCTGCTGGATAGGCCAAGCAGGGTCACTCTGACACCCAGGTT
TCCACCCCAAGGCATGGCACAATGCTGGCCTCCTGTGGGTGGAATCAAAGGCTGAGTTC
TAACAGGCTTGCGGCAGACACACACACAGAGACCACATGTACATGATGAACACACATAT
CCTTTTCATTACAGGTTATTAGTACAAGTTTTGGAATTGAGCAAACAAGAGTCTAAGCGC
TGGTTTCACCACTTCTCGTTTGTGTGACCTCAGACAAGTCATTCAACATCTCTATGACTCA
GTTTCCTTATCTTTATCACAGAGATGACACCCACTCTGACAGGGCCGAGGGAAGAACCA
TAAGCGATGGCAATGCAACAGAGTGGCACATGACAAGAGCTCAGCGAATTTGAGGGAA
TGAAACTGTAGATTACAATACTAGTACAATATGATAAACATATGATATTGTTAGTGACAT
TTATTTTACTTCTACTAGCAAATAACCTATGTTTAGGACTGACTTTAGAACAGGCTGGCA
GAAGCATTTTTGGCAGCATCAAAGTCCTCCAACCTACTGGTCTGTTGGAGCCCCCCAAGT
ACACCAAAGAGCCTCTGCATTAGCCCTGGCTGAGGGTTCAGGGACAGGCAGAGAAGTAC
AGCAGTGAGCCATCCCTGCCTGCATGGAGGTGGAGAAATGATCAGGCATGGTCAGTTGA
CAATCTCCTAAACACAGTAACCCGTGTCATACCACAGTGTAAACACACGTGCAAATGCT
TCTGCTTCCTTTCCCCATCATGAGAATAGTCACTCAATGCCGGGCATCACAAGGGATCAA
ATGCTAGGAGTACCCAATCATTCATGGATGCTTCTCAAAGGGGACGAGTGTCTAGAAGT
GTAATTTTAATTTCACTTAATTTCATATGGAATCATCTCCATTACTAATTTTGTTCTAATTT
TAATGTGATAATCACTTTGTAAAGCACAATAAACAGAGGCAGGCTCTCATGAGGAAGTC
AGAAGGAAAGAATCCCAAGAGACATGGGACAGCTCCATCCAAACTGAAAGGGCCGTGA
TTCCCAAAAGAGCAATTTTGTCCCCAAGGTCTGAAGACACTTTTGGTTGTCACAACCTGG
GGGGTTGGAGTAAGCATTACTGGTATCTAGAAGGGGGAGGCTGGGGATGTTGCTAAACA
CCCTACCATGCACAGGGCAGCCCACATTGCCACAAACTATTATGTGGCCCAAATGTCAA
AAATGCTGAGGTTGAGAAACCCTGGGTGAGGCAGACTCAGGGAGAAGGGAATCGAGCT
TCACTCACAGGCAGGCAGGAGCTGTCTGGTACTTCAACCTCCAAGACACCTCCTGCTCAT
CTCATCCTGGCTGCTCTACCCACCAGCTAGAAACCTTGAACAAGTTACTTCACTTCTTTGT
GCCTCTGTTTCCTCATATGTAAAAGAGGGATAACAAAACGCACACAACTTGCATGTTGCT
AGGAGCAGAAATGAGATAATACAGGAAAGGTGCTGAGAAGAATGCCCGGCACATGGCC
AGTTCTCAACTACTAGTCACCCATTACTATTAGTTACTCACATCTTAGAGCTAACATAGA
CATGGGCTTATTCCTGGATACACAGCACTGTCCCCATATCTACAGTGGTGATCCTAAGGG
CAACATGGCATCACCCAAATGTCTTGTTAGTCACTACAGAATCACAGTGTGAGGGATGA
AGGCCATCAAGACAGAGCTGAGGCTGGCAGGGTGGCTCATGCCTATAATCCCAGTGCTT
TGGAAGGCTGAGGCAGGAGGATTGCTTGAGGCCAAGGGTTTGAGACCAGCCTAGGTAAC
ATAGCAAGACCCCATCTACAATTAAAAAAAAAAAAAAAAAGACAGAAAGAAAAAATAG
CCAGGCGTGGCATGTGCTTGTAGTCCAAGCTACTGGGGAGGGAGGCTGAGGCAGGAGG
ATTCCTTGAGCCTGGGAGTGTGAGGCTGCAGTGAGCTATGATGGCATCGCCGCACTCCA
GCCTGCATGACACAGTGAGACCTGGTCTCAAAAACCAAATAATAATAACAGTAATAAAA
GCTGGAAAGAGCTCAAAGTTACTCATTTGACAGATGTGACAGATGAAGAAATAGAAGCG
AGTTAGGTGCCTTACCATGGTCAAACAACTAGTTCGTATCAGACCCTACTCCAGAAACTA
TTCCAGTCCGGGTAACCTCTCGTTAACCTCTCTTGTTAGAAATGCAAATTTCTGCCCAAA
TCAGGCCTCAGGAATCAAGAGACTGTGGGGTCGGCTCTGCAGGCTATCTGAATGAGGCC
TCCAGGGAAATCAGATTCACTCTCAAGGGTGAGACGATTTCCCTAAAGGAACCTTCTCAT
AACAGCCTCTTCCTGTGGCCTTTACAGGATCCTGGGTGAATAAATTCCCAGTGGAAATGA
CACACAACCACAACTTCCGGCTGAATGAGAGAGAGGTAGTTAAGGTTTCCATGATGCAG
ACCAAGGGGAACTTCCTCGCAGCAAATGACCAGGAGCTGGACTGCGACATCCTCCAGCT
GGAATACGTGGGGGGCATCAGCATGCTAATTGTGGTCCCACACAAGATGTCTGGGATGA
AGACCCTCGAAGCGCAACTGACACCCCGGGTGGTGGAGAGATGGCAAAAAAGCATGAC
AAACAGGTATTTCACACTGTGTGTTTGTTCTTTTGAGCTCCCAGATGCTGGGGGTGTCTG
GGAATACTGGAAAATGGATCATTTTTTTAAAAAGGGAGAATTATGTACAAGTACCCAAG
AACTTCCATACAGGGCCACTCTGTTAATTCAGCCCCAATTTGTTGCTTGAGATAAGAGAT
GATTAGAGAGCATTCATAAGGGACACATCTGCCCTCTAGGGGCCAGTTTCAGAAGTTAG
AGGCAGATGACTTAGAGACAGCTTGGTGCTTGCTTTGTGGCTTCGAGTCCCAGCTTCATC
ATCCCTAAAATGGGTATAATTCCATTACTTCCCCGGGTCACTTGAGAAAATAACAGAATC
AGCGATGCTGAGCGCCCCTCCCAGTACTTGGAACCTAGGAGGCACTCAAAAAAAGATTG
GCTCAACTCTTCCCTGCCCAGGAAATTCCAAGGTCCTCTTAGCCTACCGAGGACACATCA
TTCATGATTTCCTCTATTATTATTCGTTACTTTGTAGTTAAAACTGCAGGTGTTAAGTACT
TATTGAGATTATTATTGGGTCATGGCAGAAAGAATGGAGAGGTCTTATTTCTGTCTTACT
GGATACTGGCTAGGCCCATATGAAGAAGTGATTCTGGTTTGAACCTCCTTATAGGACAA
GAATACAAACATATGCAACCAAACTGAGAAAAGTAGGCTCTCAGAGGAAGGTATTTGCC
CGGGTAGCCAGTCATCATGCTCTGTGAATTTTTCCTTAACAACGTCCCTTCTGTACCTGCC
TCCTTCCATTCCTCCCTGCAGCCCGGCAGCTCTTGAGAAAGGGACTGCATCTTTTTTTTTT
TTTTTTTTTTGAGACAGGGTCTTGTTCTGTCACCCAGGCTGGAGTGCAGTGGCATCATCAT
GGCTCACTGCAGCCTCAACCTCCTGAACTTAAGTGATCCTCTCACCTCAGCCTCCTGAAT
AGTTGAGACTACAGGCGTGCACCTTCATGCCCAGCTAATTAAACTTTTTTTGGTAGAGAT
GAGGTCTCGCTGTGTTGCCCAGGCTGGTCTTGAACTCCTGGCCTCAAGCAGTCCTCCTGC
CTTGGCCTTCCAAAGTGCTGGGATTAACAGGCGTGAGCCGCTGTGCCTGGCCCATTTGAC
TTTTAATTGAGATCTTACTTGGTGCAAGGTATGAGCTAGGTAAAAGAGTGAAGAAGATC
AAGCCTTCCTGCCCATCCAGCTGGGATTGCACCTTAAATCTCTTTATCCCCTGCAAAGTG
CCAGACTAACTCCACAGGCACTACTGTTGCTATCCGCCCCCTTAGGGATTGAGTAAGTTG
AGGCAAAGATTGAGAATATTCAGCATTGTCTAGTATATACAGGAAAGGTTCTTTTTAAA
AGTACACTACCAGATATTCGACTCCTTAATTACAAAAAAAAAACCAAATGCCTAAAATT
GGGAAACCAAACCAGAGAATTATTTTAGATGCCTTTTTAAACCATAAACCAGGAAAAGT
TCTGCTGCTAACCTTGAAGATAGGAAACGAACCATACAGTCTCAAGGAAATAATCATGC
AACAGAAAACACACCTCAGTTTTCAGTAGCGGAATTACAAAGGAGTGTGCTTCCTAAAA
TCCTCAACTGACAGTCCCGGAATATAAATTTTAATAAGTGCTATATCAATTCTGTGATAA
ATATAACCCGTGGCCCTTTAAAGGGAAAATCATGATTCTTTTGTAACTTGTGGTTCAATA
AAACTGGGCCCCCCTTTCCTTTTCTGTCTAGAACTCGAGAAGTGCTTCTGCCGAAATTCA
AGCTGGAGAAGAACTACAATCTAGTGGAGTCCCTGAAGTTGATGGGGATCAGGATGCTG
TTTGACAAAAATGGCAACATGGCAGGCATCTCAGACCAAAGGATCGCCATCGACCTGGT
AACCACTCCCTTGTCCACCCCCGACCCGTCCCCAGGGTCTGCCTCAGCACAGCCCCACCT
CCACTTGCCCTTCCTACCCACCCCCCAATCTCATGTCCCAGCTTGGGGTGCTGAGTCTGCT
CTTCGGCCTGGGTGGGATACACAGAATGCCTAGTTTCATGGATGCCAGCTGGAGAGCAC
GGCACCTGGCAGACACTTACTGGGCAGGGGGGATCCCAAGAGCAGCCATGGGGTGAGC
CCCACTCCCGCTGACACCAGAGACAGGGGAGACATGTGCTGCGGTCTGGGAAATAGCTA
CCCCCAGCCAAATCATGAAAGAGCCATTAAACACCGCACTATACACATACTTAACTTAA
ACCAATCGGGCGCTCAGCAAAAGAGAGAGAACACCAGTCCAAACAGTGCAGCAGACCC
AGTTCCCCATCCCGGAGAAGTGCGCAGCAGTGTGGGGAGCTGGAGCTGGGGTGGCTGTC
CTGCACCAGCCCCCACGACCCTCAGACCACAGGCACTGCCAAGAGGGAACATGAACCTA
GCCGGCCTCTAAGTGCAACGGCTGCCCCTGACAGGTGGTGACAGATATTTTCAAGAGTG
ACTCTGACCAGCTGTGATTTCCACCTTACATGTTGTCTTTGGATCCTTTCCCTGAATGATA
TGAGATTGTGCTGGGAACTCTAGCCCTCTGTGTGCTGACCTCCAGAATCTGACAACTTTC
CTTTCCAAACAGTTCAAGCACCAAGGCACGATCACAGTGAACGAGGAAGGCACCCAAGC
CACCACTGTGACCACGGTGGGGTTCATGCCGCTGTCCACCCAAGTCCGCTTCACTGTCGA
CCGCCCCTTTCTTTTCCTCATCTACGAGCATCGCACCAGCTGCCTGCTCTTCATGGGAAG
AGTGGCCAACCCCAGCAGGTCCTAGAGGTGGAGGTCTAGGTGTCTGAAGTGCCTTGGGG
GCACCCTCATTTTGTTTCCATTCCAACAACGAGAACAGAGATGTTCTGGCATCATTTACG
TAGTTTACGCTACCAATCTGAATTCGAGGCCCATATGAGAGGAGCTTAGAAACGACCAA
GAAGAGAGGCTTGTTGGAATCAATTCTGCACAATAGCCCATGCTGTAAGCTCATAGAAG
TCACTGTAACTGTAGTGTGTCTGCTGTTACCTAGAGGGTCTCACCTCCCCACTCTTCACA
GCAAACCTGAGCAGCGCGTCCTAAGCACCTCCCGCTCCGGTGACCCCATCCTTGCACACC
TGACTCTGTCACTCAAGCCTTTCTCCACCAGGCCCCTCATCTGAATACCAAGCACAGAAA
TGAGTGGTGTGACTAATTCCTTACCTCTCCCAAGGAGGGTACACAACTAGCACCATTCTT
GATGTCCAGGGAAGAAGCCACCTCAAGACATATGAGGGGTGCCCTGGGCTAATGTTAGG
GCTTAATTTTCTCAAAGCCTGACCTTTCAAATCCATGATGAATGCCATCAGTCCCTCCTG
CTGTTGCCTCCCTGTGACCTGGAGGACAGTGTGTGCCATGTCTCCCATACTAGAGATAAA
TAAATGTAGCCACATTTACTGTGTATCTGTTATAATTCTCTATTTTTTGAAGCTCAAATAT
CAAAAGCCAAATCCAAATTCCTGGATAACTCCAGGTATGATAAAGGCTGAGAGGAAGTC
ACTTGAGCACCACAATGTGCCACAGCAGGGCATGTTCTCAGGACAGGACAGGTGTGTGC
TGAATCCTGGGGAGGGTCTGTGCAGTACCCCAGAACTGTGGGGTGCTAAGTGGCACACA
AGCCCCAGGGCTCCCACAGTCTATGCCAGGCTGCTGCAGCTTTCATCCCTCATACCTGGT
CCTGCAGTGGGTCTGGTTTGACAGAGCAGATGACACCTGAGGAATATGTTTCTGGATCCT
TCAATCCCTGGGTAAGACAAGTGAAATCCACAGAGGCTGTTCAGCACGCAAGAGTGCCA
GTGCTCTTTCAGTGAGGGGATGACTGACGGTCACAGGTGCTGTGTGTGCAGGTGTCTAAC
TGTAACCCCCACAGCCTGGCAGATGAGGAAGACAAGGGTTGGAAGAGTTCTGAAACCTG
TCCAAGATGCTGAAGTAGTGGGGCTGGGTTCAAGTGCAGGTTGGCTGGACTCCAGGGAC
CACACAAGGAGTCCTGTCACAGGCTTCTGACCCCATGAGACCAATACCAGTAAGAAGAG
TGGTAAAAGGGAGTAGGGACGGAAGGGGAACGTCACTGCCCTTTGTAGGCATGCCTGTG
GGTTATCTCACAGAGTCTCCTTACCCTCAATCCCTAGGGGGCTGGCACTGTTACCCCTCC
TTTTTACAGCTGCAGAAGCAATTTCAGCTCACAGAAGGGAAGGCCTCTGCCTGAGGCCT
GAATCCACACCCAGGCAGGGGGACCCTGCAGCCCTGCTTTCCCCTGCTCCCTTCCTGACT
TCCCACACTGGGCTCTGCCTCCTTACTCTGCTGAGAGCAGATGGTGCAGGGGCTGGATGA
ATTGCCCCAAGCCATCCTCTCGGCTTCCTGGTGAACCCTGATGCTGCGGATGGCCCACTC
CTTCAATTCATTCTCCAATCTGCTTCACCCCTCTTCTTTTCTGTCATTCTCCAAACTGCTTC
ACCACTCTTCTTTTCTGTCATTCTCCAACCTGCTTCACCACTCTTCTTTTCTGGTGCCTGTC
CTATATTTCTCATCTTGCTGCAGCTTCCTTTTGGCTCTTCTCATTTCTAAATGTAATAATCT
CAAAAAACCCTTTTAGTCCTTTGCCATGTCTGTCCCATACCCAGAAAGGCAGTGGTCACT
TCTGCTCACCCAGCGCCCTCTCTGCTACAGCCGGTGTGGAGTCCTCCACACTCTTGAGCA
TCCAGACACCCCCGTTTCAATGCCTTTTGTTCATGTACACCCACTCAGAATCTCTCAGATC
CCCTCTTACAGAAACTAGCCCATCTGTTACTCAAAGCAGGAGAGTACTCATTCAGAACA
CAGGCTCTGAGCCAGGCTGCCTGGTTTGAATCCTGGCTCTGCCATCTAGTAGCTATGAAA
CTCTAGTAGCAGGTTCTGTGCCTCAGTATCCTCATCTGTAAAATGGGGAGACCAGCAGCA
CTTACCTTGAGGGATTGCTGTGAGGATTAATCAAATTAATGTCTAGAAAGCATTTATTTA
TTTATTTATTTATTCATTTATTTTATTTTTTTGAGACGGAGTCTCGCTCTGTTGCCCAGGCT
GGAGTGCAATGGCACAATCCTGGCTCACTGCAACCTCCGCCTCCTGGGTTCAAGCAATTC
TCCTGCCTAAGCCTCCCGAGTAGCTGGGACTACAGGCACGTGCCACCACGCTTGGCTAAT
TTTTGTATTTTTAGCAGAGATGAGGTTTCACCATGTTGGACAGGCTGGTCTCGAACTCCT
GACCTCAGGTGATCTGCCCACCTTGGCCTCCCAAAGTGTTGGGATTACAGGTGTAAGCCA
CCATGCCTGGTCTGGAAAGCATTTAGATCACTGCTTGGTTTTAGCAAGAACTAGGAAAG
GTTGTCACATTATTCTCAATCTAAGAGAGTACATAAGCCAGGCC
(ZPI)
SEQ ID NO: 2
AATGTGGGTTGGAGCCCCCATACAGAATCTCTATGGGGGCACTGCCTAGTGGAGCTGTG
AGAAGACGGCCACCGTCCTCCAGACCCCTGAATGGTAGATCCACCGACAGCTTGCGCCA
TTTATCCGGAAAAGCCACAGACACTCAACGCCAGCCCGTGAAAGCAGCCAGGAGGGAG
GCTGTACCCTGCAAAGCCACAGGGGCAGAGCTGCCCAAGACCAAGGGAAGCTACCTTTT
GCATCAACGTGACCTGGACTCAAAGGAGATCATTTTGGAGCTTTAAAATTTGACTGACCT
GCTGGATTTCAGACTTGCATGGGCCCTGTAACCACTTCGTTTAGGCCAATTTCTCCCATTT
GGAACAGCCGTATTTACCCAATACCTGTAACCCCATTGTATCTAGGCAGTAACTAGCTTG
CTTTTGATTTTACAGGCTCATAGGCAGAAGGGACTTGCCTTATCTCAGGTGAGACTTTGG
ATTGTGGACTTTTGGGTTAATGATGAAATGAGTTAAGACTTTGGGGGACTGTTGAGAAG
GCATGATTGGTTTTGAAATGTGAGGACATGAGATTTGGCAGGGCCAGAGGCGGAATGAT
ATGGTTTGGCTCTGTATCCCCACCCAAATCTCATCTTGAATTGTACTCCCATAATTCCCAC
ATGTTGTGGGAAGGGACCCAGTGGGAGATAATTTGAATCATGGGGGTGGTTCCGCCATA
CTGTTCTTGTGATAGTGAATAAGTCTCACAAGATCTGATGCCTTTATTGGGGGTTTCTGCT
TTTGCGTCTTCCTCATTTTCTCTTGCCGCCACCAGGTAAGCAGTGCCTTTTGCCTCCCACC
ATGATTCTGAGGCCTCCCCAGCCACGTGGAGCTGTAAGTGCATTTAAACCTCTTTCTCTT
CCCAGTCTCGGGTATGTCTTTATCAGCGGCGTGAAAATGGACTAATACACTGTGGTTATG
TATTATAGTCATATGATATTTTCATATTTTTGGAAGCTGGGTGAAGGGTAGATGTGGAGA
CCATGATTTTTGCAAATTTTTTTAAGTTTAAAGTTATTTCTAAATTAGAAGTTTAAAAAGA
AGAAATCACATAAGCCATAACACAATAGAAAGATGTCTTTAAAGTTCAAGGCAGGAGG
GATGTCTGGAAATCAGCGAGAAATTTGCACCTGTGTGTGCATGTGCATATGTGTGTGTGT
ATGTTGCAAGGACTTGGAAAGCCCTTTTTTTCCTACCTCTGTACTACTGTGGGGGGAGGC
TAAACTTGACTTCTTCCCATCTTAGTTCTTTTTTGGGATAGACTCCTGTAACAAAAGACA
GACAAGAGAAAAATCAGCTTACAACATGGGCCATGCACTTCACACAGGAGAAACCTGC
ATGAAAAGTAACTCAAAATGGTGCCTTAGAACTCCACTTACCTTTAGTAAAGAGCAATA
AATTAGCAGGAAAATCATGGATCGGGACAAGGGAAGTGGTTTTATGCTTCCAAGGGCAG
GAAATCATGGAAGGTAAATATATGGGAGGAAACTAAAGGAATAAGGCTTGTTTGCATAT
TCCTCTGATGCCATCTCTGGGTTGATAAGAGTCTAGAGTCATTTCCAGTAAAGATGAATT
TTTATCTGTCTTTAGGAAGAAAGGGGGAAAGATAGAGAAAACTATTTCTCCATTTGCTGT
TTCTTAATTACCTTCAGTTCAAAAATAATTTTTATATCAGAAAGGCATATTTAGAGGTAT
GTTAGTTTATTTTCACACTGCTAATAAAGACATACCCAAGACTGGGTAATTTATAAAGAA
AAAGAGGTTTAATGGACTCACCGTTCCACATGGTTGGAGAGGCCTCACAATCAAGGCAG
GTCTTACATGGCAGCAGGCAAGAGGGAGAATGAGAGCCAAGCGAAAGGAATTTCCCCTT
AAAAATCCCCTTATAAAACCATCAGATCTCGTGAGACTTACTCACTACCACAAGAACAG
TATGGGGGAAACCACCTCTATGATTCAATGATCTCCCACTGGGTACCCCCCAACAACAC
GTGGGAATTATGGGAGCTACAATTCAAGATAAGATTTGGGTGGGGACACAGACAGACCA
TATCAAGGGGTAACATAGTCTGGTTTCCTTTACTACCCACCTACCCAAACACCCCCTTCA
TCTGATCCACACAAAGTAAACTCTTGCAGTTCTCTCACTGTTTCCTGGAGTCTGCTTTTGG
TCTCATAGGACTGCCCTAACGCTTGTTTTTCAGACGTTTAACCCTGTAGGTCTCTGGACA
AATTTGCTTTAGAAGCCCCTCGATGTCGCCCTGAAGAGTGGCTTTCAGAAGTTGTGCCTC
CTGCCTGAGGGGAGTTCCAGGAAGGGTTCTGCATCGCCTATGAGTTTATCTGGATCACCA
GAGGCCTTCCCGTCAGAGCTTTCCCAATCGTTTTTGGCCAAGGAGTGTGAGAAGCTAAA
GTTCATAACAACTGGAAGTCAGACAGCCTGGTCTATTCTGCTTTAACTCTAGCAGGAAAG
GCCTTCATGGTGGGGCCTGAATATCTTCCTTTATAAAATCAAAGCCTGGGGACAGGGTTA
CTTACTTCTGAGGTTCAATCTGGCTCTAAAATTATGCAACAAATGCCATTCCTTTAGCAC
TTCCTTCCTACCGGGCGAGATACTCAACTCCACAGGCACCACCTCAGTTCATCCTCTCAG
AAGTCCTAACAGCTCAGCCTGGGGCACCCCATTTTACAGATTAGTAAACTGAGGCTAAG
AGAGGTTAGGTAGCTTGTTCAGGGTCATGCTGCTGGTAAAAGAGCTCAGGCTACAGTGC
TATGCATTGAGTTTTCTCACTTTCCCATCTAACTGGAGGGCTAAAGGTCAAAGAGTGGGC
AGCTCCCTTGTTGGGAGCTGTACAGGAATAATGTCCTCCCTGAAGGAGGGGGACTTCTG
AGCCACACCCTGGGGTCCAGGGCTCACAGCCTTAGGAGCAAAATCGTCCACCCCCTTCC
TGGTTCCTCGGTGCTGCAGAGATATTCATAGGACAGAGTCTGAGTTCTGGCCACTTAACA
GAGGAAGAAAGGCTGGCTCGGTGAGGTTAACTTACATCCCAGCAGCTAGGAACCGGGA
GCAGAGGACCTCAGATTCACACCAGGGCAGGAGGCAATGGCCTGGCTGAAGCCTTCACA
ATCTTCCCAATATACTCCGCTGCCTTCCTTTATAAGGATCCATTTCTGAAACCCTGTGCCC
TGGCCAGGCACGGTGGCTCACACCTGTAATTCCAGTACTTTGGGAGGCCAAGGCAGGAG
GACCACGAGGTCAGGAGTTTGAGACCAGCCTGGCCAATATGGTGAAACCCCGTCTCTAC
TAAAAATAGAAAAATTAGCGTGGTGGCAGGCGCCTGTAATCCCAGCTACTCGGGAGGCT
GAGGCAGGAGAACTGTTTGAACCTGGGAGGTGGAGGTCTCAGTGAGCTGAGACAGACA
GTGCCTGGGTGACAGACAGAGACTCCGTCTCAAAAAAAAAAAAAAAAAGAAAGAAACC
CTGTGCCCTAAGACCTGCACACTCGCTGGCTCCGCTCAGACATTTAGCAAAGCAGACAC
CTTCCCAGGCCTGGAGGAAACAGCCCCTGCTTTTTGGGAATCCACAAGCCCGCAGCTGC
AGAGCTCGACCTGGATGGGCAGGCAAAGGCTGACTCCTGTGCGTGGTGTGAGTCCAGCC
TGGCCCCTCTACACCCTCACTTTCACCTCTTAAAGAACTGCCTATTAACAGAGCAGGTAC
TGCCCAAAAGGAACACTCTGGAAACTTGTTGGGACACTTCTGCCTTTCACAAACGTTTGG
GGGGAGTACTACTAGCATTTAAGGATTGAGGGTTAGCAATGCCAGACATACCAGAACAC
GCAGGGCAGTCTCCCATGATGAAGAGGCCGCCGGGTTCCCCAGGACTCACATGTCCACC
TCAAGTTCACGTGGGATTATCTGAGCCTAGACTGTCAGTCCTGGGGCTGCTTTATTTCAT
ATAAAAATATAATATTTATCCAAGGTTTTACTACACACTGCATTTTCTGTGAAGACAATG
ACCGTGTAAATCAGGGAAAGATCTATATTTTATTTTGTTTGAAACTTTACCAAGCATTAT
TTACCATTTCAAAAGCTCTATCCCTGGTAGTACCATTGGTTTTCTTGTTCACCGGCCAGCA
GTGAGCAGCACACAAGCGACCTCCCGTGGGCTCCACATTGGACAGCCTCACTGCACCTG
CCCAGGCCCTTAGGCCACAGCACTGCCATATTCAGGGACACATTATTCTCTTTTATTATG
CCTCCATATTATCATTACAGCATTATCTTTTTTTTAATTTTGTGGGTAGATTATATTAGCT
ATACGTTTCACTTCAATGGTAGTAGTAAGGGGCACATAACAAAATATTTACTTATATATA
TTAAAAAAGAGAGTCTGAGAAGTCTGAAAAGTTTTGCCATAAACGGTCTCCACCAGCCT
CAACTCTGAGTGCCCGAGGATTCAGTCTCAAGTCCAGCAACATTGTGAAGCAGGAAATT
TACCTTGAAAGGAGCTATGTACTCTAAGTAGTGATTTACCTGTCTGCCTCCCCCACTGGA
TTGACCAGTTCCTTGGGGGCTGAGAGAACAGGTCCTGAACATTTCTGCTGTGCCCCCCAA
CCCACATCCTCATAGTGTCCAGTACCAGGCTGGGGACTCAGGAAGCATCCATGGGATCC
CCCAGTGCCTTCTTTCTCGAGGTGTTCAGCACCTAGAACAGCTCAAGACAAATTCCCCAC
ACCCCACCCAGACAGAGCTGAATCTTACTGGGGCGAAGCCTTGAGTTGCAAGGCAGAAG
CTCTCGTGATGGGATTTGGGTCATATTCCGGGTTATAGGAGGAGCTGGGGAGTATGGGA
AGCCTCCCACTTGGTCTTTGGTTTTCCAGAAACTCCACCATCACAAGCAGGATGTTAATC
AGTAACCGTCCCACAGGGGATCATACTTTGGAATAGCAAATATTTGCTGAAGGTTCTGG
GCTGCAAAGCTGAAGCTTTGGTTTCTGCTCTAAATGAAGGACTTTTCCAGGACCCAAGGC
CACACACTGGTAAGAGGCAGTGGGTTACAGGAGACCTTCAATGAGTCTAATCAGGGAGG
GACCGGGAAGGATGGTATCATCCCTGGGCGGGCTCCAACGTGAGGGCTGTGTGGCTGAG
CAGTGCAAAGACCTCCATCCTACACTCCACAGGGACTGTACATACAGATTGGGAGCTGG
AGTGGGGTAAGAGGCGAATTATAGACACAAGGGGCTCCTCTGCAGGAAGGAGGCCAAG
GGAAAGAGGCTTGAAAGGCTTGATATTTCACCCACCACCACTCACTGCCGGAGTAAGCA
GGTCTCCCCTTCCCAGGGCTGAGGGGAGGCAGGGATGTGTGCTGTCCCAGGGCTGAGAA
GTGGCAGGTGAGCTGGTGATTCCTTACTGCCCAGGTTCTGTCTAGGAAGGTGCGTCCTCA
CCATGCTGGATGGTGTCCTAGTCCAGGAGCACCCCCTGAGCTCCTGGCCTAGACTCCAAA
GGGTTGGGTAGATGAGCAAAGACTTTACAAAGACCTTAGGCGATATATGTCCAGGAGCA
CCCAGGAATTACTGGGCTACCACTGCAGACTGCAGGACAAGCTCCAAGAACAGGAAGGT
AAGACTCAGCATTTGGAGGTGGTGACATCTAGTTGGCGTGCTGGGCTAATTTCCTGACCA
TTGTACAGGGAGAAGTAACCTTGAATTCAGGAGTATTCTGTGTGGTCTTAATGTAGAAA
GTAGCACTAAATGATGCCACGTAATCGTTTTAGCTCAGGCTCCTCTAACAAAACACCACA
GGCTGGGTGGCTCCAACAGCCATTGATTTTTCACAGTTTTGGAGGCTGAAAGTCCGAGTC
AGGGTGCCAGCGTGGCCGGATTCTGGTAGGGCTGTCTTCTTGGCTTGCAGATGGCCACCT
TCGCACCGTGTCCTCCCATGGAGAGGAGGTGCGGAGGGGGACTCTGCTCTCTTCTTATGA
CAGCACTAGTGCTATCACAGGGGCCTTGCCCTCACGACCTCATCTAAACCTAATCACCTC
CCAAGCGCCCCAACTCTATTGCCATCACAATGGTGGTTCGGGCTTCAACTTATTAATTCT
CAGGGGACACATTCAGTCCATAACAATAAAAGCGTGAAACTGGGCTGCGTTTACACTGA
AAGAGCTATTTACCCAACGTTTACAATACTTGGGTGACCTGTTGAATGCAGGCTTGCCAT
TTAGAGTCAAAAAGAGCTTCCTCAACAGTGTCCTTTGGGAAACACAGTGGAAGTATTTC
ACTGCTTCTACAGGGGAGAGGGTAGTGCCGTTCAGACTGCAGAGTGAGGCCCTGAATTC
CGGGGTGCCATTCAGCCCGAGCAAGGGGCAACATGCTGGGCCCTGGCGCTGGAGGCGGT
TTTGTCCCAGGCATAGATAAGGACTCAGCCCCTGCATCAGGAAGAGGCCTGGCAGCACC
GCCTGTCAATACATTTTGCCGCAGGTGACCTTGGTCAAGAATAAGGGTCTCTGCTGATGG
GAACTACTGTGAGGCCGGCAGCATCCACCCTGCGCTCACTGGGCTGGGTGGCCTACCCC
ACCCAGACCCTCCCAGGGCAGTGGGCCCAGAGAGAGGATGAGGGAGGGCAGGTGTCCC
AGGGGTTCTGCCCAGCCAGCCTCTGGGATCAGGCCTGCAGTGTGGCTGAACACCAGAAC
TGAGTTTGGACACAGCCAGGTGGCCCAGGCCAGTCCCAAGCCATGTATTTGGATGGAAA
ACATGGAAGTATTCAGGAGCCAGGCTCTGTGTCCAAGGATGTGGAGGGAGCCTAAAAGG
CGACAGAGAAGGGGACAGCTAACGGTGAAGAAGTGTAGCTCCCACACTGCAGCCTAGG
ACAGTGAGAACCGGCATGCAGCCCAGGTGGCTGAGGGCTCTATGAAGCCACAGTGGAG
GGAGCCCAGAAGTGGGTTGTATGAATTGCGGGGCCTCCTGCTACCCGGGAGCTGCAGCT
ATAGGAAGGAAGGAAGGAAGGAAGACCTCCAAGGAACTGTGTAGCAGAGGTGCAGTGC
AAAGAGAATTTTGATAAAAAATCCAGGAAAGCTCCAATACTTTCCCCCTTCCTTGCCTAA
CGGGCATGCAGGCACTCCAATCCCCAGCCAAACAGGGCACTGGGCAAGGCCGGCCACCC
ATCTGGATGGGCAGCCTGACGACCAGATGGTCAGGGCAGTGAATGAAGCAGATCAAGG
AAAGGTGTGTGAGGACCCCTGATTCCACCTGCTTGGACCCCCACCTTCTGTGCTGCCTCC
TGCTCCCAGAGTGGACTCTCTTGCCCTGGCCCTCAGGGAGGAGACGGGATGAATGAAAA
CGGGGTCAGGACTGAGAGCTGCCTGCCGGCCTGGCAGGGAATGGGAACTGGAGGAGGT
TTTGCTCTGTGAAATAATGTCCCCTCTTTGGGTGAGCAAATGTCACCCACACTTGCTCTA
GGTCTCCCTGGGGCAGGGCTAACCTACTTGAGCCACAGGAAGGAGGCAGGGTCCCTGAA
GAAGCTTTTACTATCCACAAAGACATTTTAGGAGGCATTAAAACCATCTCTATCCTCTCC
TCTCCACAGGAAGTCTTGCAGCTGAAGGGAGGCACTCCTTGGCCTCCGCAGCCGATCAC
ATGAAGGTGGTGCCAAGTCTCCTGCTCTCCGTCCTCCTGGCACAGGTGTGGCTGGTACCC
GGCTTGGCCCCCAGTCCTCAGTCGCCAGAGACCCCAGCCCCTCAGAACCAGACCAGCAG
GGTAGTGCAGGCTCCCAAGGAGGAAGAGGAAGATGAGCAGGAGGCCAGCGAGGAGAA
GGCCAGTGAGGAAGAGAAAGCCTGGCTGATGGCCAGCAGGCAGCAGCTTGCCAAGGAG
ACTTCAAACTTCGGATTCAGCCTGCTGCGAAAGATCTCCATGAGGCACGATGGCAACAT
GGTCTTCTCTCCATTTGGCATGTCCTTGGCCATGACAGGCTTGATGCTGGGGGCCACAGG
GCCGACTGAAACCCAGATCAAGAGAGGGCTCCACTTGCAGGCCCTGAAGCCCACCAAGC
CCGGGCTCCTGCCTTCCCTCTTTAAGGGACTCAGAGAGACCCTCTCCCGCAACCTGGAAC
TGGGCCTCACACAGGGGAGTTTTGCCTTCATCCACAAGGATTTTGATGTCAAAGAGACTT
TCTTCAATTTATCCAAGAGGTATTTTGATACAGAGTGCGTGCCTATGAATTTTCGCAATG
CCTCACAGGCCAAAAGGCTCATGAATCATTACATTAACAAAGAGACTCGGGGGAAAATT
CCCAAACTGTTTGATGAGATTAATCCTGAAACCAAATTAATTCTTGTGGATTACATCTTG
TTCAAAGGTACTTTGATAATGTTCTGCTCTCCCAAGGCCACAGGGCCCTACGATTGTCTC
TCCCTTTCCTTTCGTTAGGCCAGCATATGATTAACGCTACGTGATTTTCTATGAATGTGTT
TTCACGTTTCAAAAACAGATTGATACACATATTGAACAGTGCCAGACGCTGTCATTTGAG
GCCCTTCCCTGGTATCCTATGTGCTTGTAGTCCTTATTATTTTCAGAGCACTCTACATAGC
TCCCCTCTGACACTTAGAAGCATAGGGTCTTTCCAAAAAACAGGGGGCTGGGGGATTAT
CTGGGGGATTTAGGATTGCATCATTGCTCCTTCATTTTTACTTTTTGACCAACTCTCTGCC
CTTAGATTCCTATTATAGAAAATAGGGACACTCCACCTACTACAGTGTTAGAGGCTAAAT
GAGACAATGAATGTAAAGTGCCCAGATGGGCTTGGCACATAGCAGACACTGAGTATCTA
TTGTTTACTTGTTCTTCCAAACTGCCAATCAGCAGGTAGAGCAGGAGTTGTCTCCTTTCTA
AAGATGAAACCAGCTCAGAGACGTTAGCTTGATCAAGGTCACACAGTAAGTGGCAGAG
GCAAAACCCAAACAAGGGCCTCCTGACCCCCTGATCCTAGGTTCTGTCCAGCCCTGCCTC
CCTAATGGGGCACTGGACGTGGGTTGGATGCCACTTTCGCAGAGCTGGCACCAGACTTA
CAAAGCCCCGGCAGGGGAAGCCACTTTACAACCAGCCAGGCCACACCCCCAGGGCAGA
CGTTTATGTAGAGAGTAATGTACCTGCCTGCTAGTAGCCTCTGCATTGTGGGGCCTTCTC
TCAGAACCACACTAAACAGTGGGTGGGTGAGAAGTGTCACTCCTGCCACCTTGGACTCT
GCATGTGCTTGTGCCTGGTGTGAATGAGACAAAGTGGCAGTCAGAGGTGCCAGGCAAAG
GCTTTTCTCTAAGCTGGAGCCAACTATGAGGGAACGACTGTGAATTCCGTTCAGGTCCAG
GACAATGAGAGGAGCCAGGGATTGTTAGGAAACATTTCCCTGCTTTCGTGTGCGATTCCC
AATAGGGCCTGCGAGTGGAGCTGCATTTTGCTAGCTGGGCTAGAGGACGGGGAAAATTT
TGGGGAAATTTATTTTGCCTGCCTGAGCTGTGGAAAAGCCAACCCAATTAGGGAACGCC
TTTCCTAGTTGGAACGAGAAGACGAGAAGTGAGAGAAGTGAGATAGAAGGCTCCCTCTC
TATTATTTGAGCAAGAACAATGCTTTTCAAAGAGGGAATTTCTGCAATGAGTTCTTCTCT
TACTTGTTCAGGGAAATGGTTGACCCCATTTGACCCTGTCTTCACCGAAGTCGACACTTT
CCACCTGGACAAGTACAAGACCATTAAGGTGCCCATGATGTACGGTGCAGGCAAGTTTG
CCTCCACCTTTGACAAGAATTTTCGTTGTCATGTCCTCAAACTGCCCTACCAAGGAAATG
CCACCATGCTGGTGGTCCTCATGGAGAAAATGGGTGACCACCTCGCCCTTGAAGACTAC
CTGACCACAGACTTGGTGGAGACATGGCTCAGAAACATGAAAACCAGGTACAACTCTTG
CCCACACCCTATACAAACTCTACCTTTCTGTACTGGCAAACGCTCAGCACAATTTCATTG
AATGCACCGTGATTTAATGTCTCCTCCAGTGAGCTATAAGTTTCCTGAAGGCAGGGCAGC
ATTTGTCTTTTTTTCCACTCTATCCCCAGCATCTGTCACAGGGTGCCTGGCTGATTCATTC
ATTGAGTCCATCAGTATTTTACGTTCTGCGACTGTGATAAATATATGATGCCAGGGATCC
ATCAGCAAACAAAACAGGCAAAATTAGTCTGCCCTCATGCAGCTTACATTCTATTGAAG
GAAGACAAAGAGTAAATTAAAAATAGGTAATAATGCAGGGAAGGGGACAAGAAGCATC
ATCAGGATGCAGATGGAGGTTAGACAAGGCCTCTCCAAGAAGGTAACAGTAAGCAAAC
ATCTGAAGATGAAGGATAAACCATGTGGATATATTCGGGGAGAGAAGTGTTATGTTACA
GGCAGAAGTGTACAAGTTCTGGGATGGGAGTGTACCTGGTGGGTTTGAAGAACATCAAG
GAGACAAGTGTGGCTTCAGCAGTTGGAGATAAAATCAGAGAGGAAACAGGGGCCCAGT
CCCCAGAAAAGACTTGGGCTTTCCTGAGAGAGGCAGGAAGCCACTGGATGGTTCTGAGT
AGAGGAGCAACCTGATTTTGACTTCTGTTTTTAAAGGATCACATAAGCTCCTGTGTTGAG
AAAAGACACTAGGGGGTAAGGATGGAAGCAAGGGAGAGTGGTTAGAAAGTTACTAGCA
ATCCAGGTAGAGATGCTGCTACCTGGACTGCGGTGGTGGTAGTGGAAGTGGTGAGAAGT
GGCTGGATTCTGGATCTATTAGGAAGTGCAGGATCTGCTAATCGATTGGATGTGGGTGA
GAGAGGTGTCAAAGGTGATCACAAAGTTTTTGGCCTTAGCAACTGGAAAGACGGATTTG
CCATTTACTGAAAGGGGGAGGAACAGGTCTGGGGTAAGTGCAGAAGTTCAGTCTTAAAC
ACTTGGATCAGAAATATCTATTAGACATCCAAGTTGAGATGTCAAGACGACAGGTGGAT
CTGGAGTCTAGGGTGAGGTCCAGGCCGGAGATATAAATTCGGTCATCAACACAGAACTA
GAATCTAGACACATGACAGGGTTGGGGTCTGTAAATATAGAGGAGAGGAAAAGAAAGC
ACAGAGTGGGCACTGAAATGTCTGCCCAATAAATTAATCCACCTATTGGAGTACAAGGA
AAATGGCTGCAATACGAATTCCATGGCTATGGCTTCTGAATCCTGTGACTCAGATTTTGG
CAGACAAGTGCAGCTAAAGGTCCCCAGGGTTAGTTTTATCTTCATTATTCTTCTTTCATTT
TTCTTCATATCTTTAGCACCTAACAATGAACCCCAAACATCATAAGCCCTCAAGTAATGT
TTGCTGAATGAATAACTTTTTAAATTAATCTTCAAGACACGTCATGTCCTCAATTATTTTT
AAATAAATAAAAAAATTTTATTTTGAGCCACAGAACTCATCTTTTCAAGCAACATATTTT
CAAAGGAGGACTCCAGTATACAAAATAGATGGTATCAGAGCTTCTCTGGCTAAAGACGG
GTAGGGGTTGAAAGTTTTCTTTGCTCCCCTCCCCATCCATCCCCAGACTCCTCGGGTCTGC
AGAATCCAGGAGCTGAAAACAGCCATCATCCAGGAGGCTGCAGGACTGCTGAAAGCAG
CTGTTAACTCAGGTTTTTTTTAAAATATAGGGAAATGAACACATAAGTACTTTGCTAAAG
AAAACGTGAGTCACTGGCTGAGGAATAAAACTCATTCACTGAAGCTGAAGTACTATTTG
ATAAGCTAGAAATATTTTCCCTGAGTAGACCACTGTAAAAGAATGGCATGAACTACATA
GTCAACTGAAAGACTCATTAATGGAAATAATCTTAAAGAACAAAAATTGTGACCTTTTT
GGTGTCCACAGACTAGGGCTTTGTCTACATTTCACCATCATCTGTTCTTGTACCACAGAA
ACATGGAAGTTTTCTTTCCGAAGTTCAAGCTAGATCAGAAGTATGAGATGCATGAGCTG
CTTAGGCAGATGGGAATCAGAAGAATCTTCTCACCCTTTGCTGACCTTAGTGAACTCTCA
GCTACTGGAAGAAATCTCCAAGTATCCAGGGTAAGTCAGGATCTTTCATCAGAGCCCAA
CCTCAGCATGAAATGTCACCAAAACAAATGCTTTTACAAACCATTTAACTTTGATAAAAT
ACCTAATTGTAGTGGAAAATTAGATTTAAGTCCCAAATACTTGAAATAGCACCCAGGTT
GGATGTTTTAAGAATTTCAAGCAACTTCATTAAAATAACTTTTCAACTAATTTATTTTAA
GCAGACCTCTCCCCCTCTGCTTAAAGTGCTCAGGGAGAAATTTGACCCTGAAATAGAACT
GGTTTACAGAGGCATCATCATTTATGTTGAATACAACTTGAATAGTTCATGAAATTACAC
CACCTTTACAATGAAACAAACCCCTAGACATCATCTAGCCCAACTTCTCCCTCCTTGTGG
AAATCCCCTCCATAGCCCTACGAAATAGCCCTCCAACTTCTCTTCCTCTTCATGCTTCCAG
TGACATCAAACTCACCATTTCTTTGAAGAGCTGCCCAATCCACAAATAGCTAAAATTGTT
ATATGTATATATATATATGTGTGTATATATATGTATATATGTATGTGTGTATAAATGTATA
TGTGTGTATATGTGTGTGTGTATATATATATACACACACATATATATATATATGGAGAGA
GACATACATATATATATGGAGAGAGAGAGAGAGAGAGTCCTGTAACTTCTGATTCATAC
TTTTTGGTCCTAGTTCTATCTCTAAAACTTCTAAGAACAAGTTTAGTCACCATCCACATAG
AATCCCTTCAGTTACTCAGTGTTTCTCAGTGGAAGGGTTCTTGGTTTTGAGGGGAACTGC
TTGTTGTCCAGAGCAGTTGTGCATGTTGCAGGGAACTGGTTAGCATTGCTGGCCCATGTT
CACTAATGCCAGTAGGAAACTCCAGTCATCACTATAAAAATGCTCCCACACATTTCCAA
ATGGCAGCTACATCTCTCTACATTCTTCCTTAGCTGTGTGGTTTAATATTTTCTTATACAA
TTGCAATTTTCAATTCCAAGAGAGACTAAAAATGGCATCCACTTAAGTAGGACACAGTA
GGGTAACTGTGGCCTGGAATCAGGTCTTACAACCTCAAGAGAGGTAAGACAATTAAATA
AAACAATCCGTCAGACCAGCACCTGAAAGTGTTTCTGCTATGAACACATGAAAAACTGA
AATGCGCTGCTGCTTTATGAAGGGTCATCATGAAATTTAAACTGTAAATGATTAAATATT
CTCCCTCTGTTTGCTCTGGGGAATTAATTTTCCTCTAGGAAATCAGGGAATTTCCTGGAG
TGAAAATCAGTGTAATTACATGTTATGTTTTCATTATCTCTTATAACACAGTAATTATATA
GGTACATCACTCATATCACATCTTGTTTCTGTAAAAAAGGGCCTCCCAAACATAGCAAGC
AGCCACAGTATAGGCAGCCAGAATTCAGGAAGGCTCCAGGGACCCCTGGGCTTGGCCCA
GAAAAATGCCTCAGAGTAGTACCAGGTGCTGGGAAGCTGCTACAGAAGACTAGCCATTC
CCTGCCTCCACCTTGCCTGCCAAAAGGAAAGTCAGAGGACTCAAGGGATCCAGGGATCA
AGGGATCCAGGCAGCTTGAAAACCTTTTAGGAGCACCAGCTCAGCTCAAGAATTAGTAG
CATAAATTACATGCTCAATAAAGATTTGATGCATGAGTGCATCCTGAGTCCATGCCCGGA
ATGTGTTTCACATATTCCACAATACTTCACATTGGGTTCCTGAGGTCTCCTGGTATTGTTT
AAGACTCCTGTGGCAGTCCCTGGTGCAACCCCAGACCACTCCTCTTAACGTAGATGGGCC
TGCTCCACTAAATCCCAGGAGCATGACCCCATGGGTAGGACCACTGTGAAGAATTTCAA
GGGGCTCATTTAATTCCTCCTTTGCACTGCCACACAAATGGTTTTTCACATTATTTCCTTT
TTCCAGGTTTTACAAAGAACAGTGATTGAAGTTGATGAAAGGGGCACTGAGGCAGTGGC
AGGAATCTTGTCAGAAATTACTGCTTATTCCATGCCTCCTGTCATCAAAGTGGACCGGCC
ATTTCATTTCATGATCTATGAAGAAACCTCTGGAATGCTTCTGTTTCTGGGCAGGGTGGT
GAATCCGACTCTCCTATAATTCAGGACACGCATAAGCACTTCGTGCTGTAGTAGATGCTG
AATCTGAGGTATCAAACACACACAGGATACCAGCAATGGATGGCAGGGGAGAGTGTTCC
TTTTGTTCTTAACTAGTTTAGGGTGTTCTCAAATAAATACAGTAGTCCCCACTTATCTGAG
GGGGATACATTCAAAGACCCCCAGCAGATGCCTGAAACGGTGGACAGTGCTGAACCTTA
TATATATTTTTTCCTACACATACATACCTATGATAAAGTTTAATTTATAAATTAGGCACA
GTAAGAGATTAACAATAATAACAACATTAAGTAAAATGAGTTACTTGAATGCAAGCACT
GCAATACCATAACAGTCAAACTGATTATAGAGAAGGCTACTAAGTGACTCATGGGCGAG
GAGCATAGACAGTGTGGAGACATTGGGCAAGGGGAGAATTCACATCCTGGGTGGGACA
GAGCAGGACAATGCAAGATTCCATCCCACTACTCAGAATGGCATGCTGCTTAAGACTTTT
AGATTGTTTATTTCTGGAATTTTTCATTTAATGTTTTTGGACCATGGTTGACCATGGTTAA
CTGAGACTGCAGAAAGCAAAACCATGGATAAGGGAGGACTACTACAAAAGCATTAAAT
TGATACATATTTTTTAAGATGTTTGTGCAATCTGTCTGGTATTTTAAGCTTGTTTCTAAGA
ACCTTAGTTACTTGGCTAAAGACTAGCTGGGTAGAATATCTTTTCTCTGTTGCTCACATAT
TTTCATTTTTAAAAAGTTGCAGATGAGAACACTATGTCAAGATAAAGCCTTTGGGAGGA
ACACATGTAAACATTCTCCTTGAGTCATGTGCTTCTCTCTCTTTCCTTCTCTCTGGTGCAA
AATAAGTGTTTTATTTTAATCTATTACGGAGTCATTTCTTGCTGACTGACATCAGAAGAA
AATAGCTCTAACCAGTCCTGATCACAGCATCTGCTTCCATGGTGCATCAAATCGCTTGGC
AGAGGCATTGGCTGAATCACAGATCATCTAGTTCAATACCTTCATTTTACAAAGGAAAG
AAAGAGGGACCCAGAAACAGGTCCATATTCTTACTTTCATGGGCCCTAGGCACGTTTAA
CCTTGTAGACTCCTCCTTCCTTCATGAAGATATATATGTTCTATGGCTGCATTGGTAGAA
AGATGAATATATTCGTCTTTCAAAGTTGCATATCTAGCTTCAAAGTTATATGTCTAGCAT
ATGGCAATAAGCAAAACACCTTCATGGGCCCTTACAGTACTGTCAGCCTTGGGCACTGT
GTCTTCTGCATCTAGTGGATAAGTCATACCTTATATACCAGTGGGAACAAAATACTTGTC
CAAGGTCTTCCAGTGTGGCAATGGCAGAGTCAGAAGCCTACCTTTCCTGAGTCTAGTCTC
CAAGCCCTTTTTACTCTTCCTTCCATCTAAAACATCTGATGGGGACCAGGTAAACAGCAT
GCACTACAGCTACCCATGGGGGTTAAACAGAATATAAGCATGAACTTTGTCCCAGGGTG
AAAAGGAAAATCGTAAATATCCCTGATCTTCCTTAGGCAGTTATTTTCTGTCACAGAAAC
AGAAAAGACTATATTCAGAGAATCCTGAATAGAGCTGATTTACAGTGTGAACTATGTTA
ACTAAATGCCTAATTGGATTTCTGTCTGTCTGCTATCTAATGTTTAAAAAAACCTAAAAT
TCATTTATTGATTAGTTGTTTAATATAATTCAGAGTAATGTGAATAGGTAATAATATTAA
TATGCAGTCTAAATACTGACTTTTCATCATTCCATAACCTGGACTGATGAAAAGTCAGTA
TTTAGACTGCATATTAATAAAATAAAATTCATTCCTGTATTCATTCCAAGAGTACTAATT
GACACTTATGAAGGGACAGGCAATTCTAGGCCCTAGAGGGCCAAAGACAGAGGACTAA
CTCTATCTGACATTCTTAAGTCACCTTGTTTGTGTTCAATTAGTCAGATTTGTTTGTGGAA
AAATAGTAGAAAGAGGAATAAAGTAGCATCCAGTCCAATTTCCCACTTTTAAGAGATGA
AATCTGGAAAAATAAGTCTGTGAGAGCACAATACTCACTGAAATCAATATGGCCAAACC
CAGTAATAAAAAGGTACATTATTATTGAAGGATTCATATAGCATGCAGATAAAAAACTC
CTGCCTTCTTCCCACCACATACACTGCAAAGCAACAACAGCATAATAATTGTATTTAATA
TACTACTCTTTAAGGTAGAAAATGGACCTATTCTATATTTTAAATATACTTTTTAATGTTC
CCTCACATTTGCTTTAAGAAGTTCCTAAGACACTCAGTTTCAGATTTCCCAAGTACACAG
GCATGACAGAAAAACGCAGACCAATAAAAAATGTAACTTACCTTACACAAATACATACA
CACAAATTCAGGGTTTCCAACCGAGCGGGGGAAATCTTAACATTGTAGAAGTCTTCACT
ATATATGTGTCGAGTTTTTGTTTTTGTTTTTGTTTTTGTTTTGAGACAGAGTCTTGCTCTGT
CACCCAGGCTGGAGTGCAGTGGTGCGATCTCAGCTCACTGCAACCTCCACCTCCCGGGTT
CGTGCCATTCTCCTGCCTCAGCCTCCCGAGTAGCTGGGACTACAGGCACCTGCCACCACG
ACCGGCTAATTTTTTGTATTTTAAGTAGAGATGGGGTTTCACTGTGTTAGTCAGGATGGT
CTTGATCTCCTGACCTTGTGATCTGCCCGCCTCGGCCTCCCAAAGTGCTGGGATTACAGG
CATGAGCCACCACGCCCGGCCAAGTGTCGAGTCTTAAAAATTGTTCCTACACAGACACA
CTCAACCACACGTTCTCACATATATATGCTGTAACAACTGAGAACAGGTTACTGACTTAA
TCTAATTCATTCTATCTTCATTGTAAAACTTCCACTCCAGCTGAAGAGCCTGTTTCATTTC
AATTCAAAGATTTCTCATATATCCACTAATTGTATGGCAAAACTGACTCATCTCCAGACT
AAGATATTCAAGCTCAGGAAGTCAAATAATAGAAATGATTTTTTAAATGTGTAAGAGGT
TATAAAGAAAAACTTTATGTGCTCCTTATTTAACCTCTATTAAGTAAAATCCTTTATAGA
CCTATCTCCATTTCTGCAGTAAAAGTGAGCTCTACAGTTAGCTTGTAAGGCTAACTAGTG
AAATTCCTGGACTTGTTCTTAAAAATGCAAGTTTTAGTAATTAACAAAATGATGATGAAG
ATGTCCCTTTTCCCTACAACTACAGATGGAGGGAGATTTTTCTTTGCCATACAACTAGCT
TAAAGGATTAATTTGATAAGTTGTTAAACTGAGAACTTTCACAAAAGTATCCATCTTGTT
TTTGATATAAATGGAGATACATGTAGTTATTCATAACTGTCAGTAATTTGCTGTTTATCCT
GTTTCTATATATCTGTCCTTGAGAGTATAATTTTAATAAATATTTCAAAGATTTTAGGAA
ATGTCATGTTCTGTTAAAAAACTTCCAAAAGTAATTTTGATGAACAGTTTTGATAACTTA
GTACTAACTAGGACTAAGACTGCAATTGACTGCTCTACATTCCTGAACTTTATAAGCAGT
AGTTGTTTCTCTCTGTCAAATCAGTGTCCCCTTTTCCCATTTGCATCATGGGAAAGTGAAA
CCTTATAATTCTGCTAAATTTATTATAACAAATACATTGAAATTCTCCATTTTATTAAATT
AATAGAATGTTATGAATCAAAGCACCAAAAAAACTGATGCAATTTTGATGTCTCGTTCTG
TACCACATTCTCCAGATCTTAATATATTCAGTTCCACATTATTGGTGCTAGTAGGAGACA
TAATGAAAACAGTTAAATGAAATCCACAGCGAGTATACTGATTAACCAGTACTGTCAAA
TTTCTCATACCTATTGAATTTTAACTACTGACAAAATGAGCAGTAACAATTCCATTTACC
TGATTGTCCTTTGGCAAAGGATATTATTAAGAATCACTAAAAATAGCCATAAAGAAGCC
ATATGGAAGGAAGAAGGAAAACAAATGGCATGAAAAGGTCTCTCACTGAGTAACTATG
CTCTTATAGTTGACGCTGGTATATTTCTTTTATTCACTACCTAAAAATGAACTATCTTACT
CTTTAATTATAGAATAAAAACTGCAGGAAAGTATTTAAGACTTTTTTTCACAAACACAGG
TATCTCATTAACCTATGTTTTATTTTGAGTAAATTCATTATTCATTATTTCACATTATAAA
AAGTAACCACACATACATATGCATTCACAAATTAGATCATCTTTATCATACATCAATATA
TTTTAAAAAACAAATATCTTCTAATATCAATATAGTTATATGCTGATTGCATTTTGAAAT
AGAGAAGCTGACAATAGCTTCACACGGTATATCTCAAGAACTGACAGTTTAAAATTAAG
AACTGTATATATTCCACAGGCAAATTTTGATGGAAATATTAGCATTAGTACAAATAAATG
CTGTTGACATAGCTTAAGCATGATAGCTTGGAATAACAGCTGATTCAGACTAGATTCATC
ATTTTAAATAAAGACAAGTACAATCTAAAATGTAAACAAAGTATTTATAAAATAAATTC
TCTAGGAAATAAAGAAAATCATCAATCTATTATTTTTAAGGTATTTATAGCTCAAAGTTA
CCAGAAATCTTTGTGGAATTTTCACTGCCAAATTTAAATTTGGGAATGTCCGGGTACAAC
ATATTGTCACCACAATCCGGAGGGCCGCCAAAATCGCAGACGGCTATTTGCATCCTTTCA
GTGTGACTTTTCAAGTGGGCTTGGAGACTCATGAGAAAATGCAGTATCTTTCTCACCTTC
CAAGTCCCCCTCCAAGTGCTTATCAAGCTAGGACAATTCAGCTGATGTAGACTTTCATAC
GATTTTTAAATGCTAAAACTCTAGAACAATTAAATGGCTGGTTTCCTGCACAAATAAATG
CAGACTTGTCTCTTTTGCAGCAGTGGTTAAAGCACATTCCTAGAGATGTTTTTCATTACA
CTTCACTATAACATTGGAATTCCGTAACCACATTATTACTCAAGAAATATATATTATACC
TCCTAGGGAATCTAATTTGAAATATGAAAAGTTTAACATCAGCTGTCATTATGTCTCTCT
TTCTGCTCATTAACAACAACAAAAAAAAAAACCCAAAATTTAAAAACAAAGCCCCAGCC
ACTGCTTTAGCTTTTGTGTACCAATCACATTATCTCCTGCTGCCTTTGTTTTGCCTCCTTCA
TCAAGCAGTTGATTTAAGGATTGGATTTTCTGGATTTTCTTTGGGAAGAAAGAAATGAAG
GAAGAGAGGGAGGGTGGGGAAGGAGGGAGTGAGAAAGGGAGAAAAAGAAAAAAATAT
GAAAAATGTTATTCATATAATGTGTACAAAGTAAATTAAAAATATATAGATACTCTACTT
TGAATAATTCTAATATATGAGAAGT

Following Table 22 provides oligonucleoside mRNA target sequences of HCII and ZPI, together with the corresponding positions in transcripts NM_000185.4 and NM_016186.3.

TABLE 22

| | | Starting | |
|---|---|---|---|
| | | position on | |
| | Oligonucleoside mRNA target | reference | Reference |
| SEQ ID NO | sequence 5′→3′ | sequence | sequence ID |
| SEQ ID NO: 3 | GGUGAAUAAAUUCCCAGUGGAAA | 946 | NM_000185.4 |
| SEQ ID NO: 4 | AACUGCAUCUACUUCAAAGGAUC | 920 | NM_000185.4 |
| SEQ ID NO: 5 | CAACUGCAUCUACUUCAAAGGAU | 919 | NM_000185.4 |
| SEQ ID NO: 6 | AAGGGAGAGACCCAUGAACAAGU | 557 | NM_000185.4 |
| SEQ ID NO: 7 | UGGGUGAAUAAAUUCCCAGUGGA | 944 | NM_000185.4 |
| SEQ ID NO: 8 | CUUCAGGAGGAAUUUUGGGUACA | 676 | NM_000185.4 |
| SEQ ID NO: 9 | CAGCUGCCUGCUCUUCAUGGGAA | 1501 | NM_000185.4 |
| SEQ ID NO: 10 | CUCACCAAGGGCCUCAUAAAAGA | 854 | NM_000185.4 |
| SEQ ID NO: 11 | GACCUUUAUAUCCAGAAGCAGUU | 716 | NM_000185.4 |
| SEQ ID NO: 12 | CUCAACUGCAUCUACUUCAAAGG | 917 | NM_000185.4 |
| SEQ ID NO: 13 | AAGAGCCGGAUCCAGCGUCUUAA | 407 | NM_000185.4 |
| SEQ ID NO: 14 | GGAUCCAGCGUCUUAACAUCCUC | 414 | NM_000185.4 |
| SEQ ID NO: 15 | GGCAAAAAAGCAUGACAAACAGA | 1191 | NM_000185.4 |
| SEQ ID NO: 16 | UUCAGGAGGAAUUUUGGGUACAC | 677 | NM_000185.4 |
| SEQ ID NO: 17 | CACAACCACAACUUCCGGCUGAA | 974 | NM_000185.4 |
| SEQ ID NO: 18 | AUGGCAAAAAAGCAUGACAAACA | 1189 | NM_000185.4 |
| SEQ ID NO: 19 | GUGAAUAAAUUCCCAGUGGAAAU | 947 | NM_000185.4 |
| SEQ ID NO: 20 | GGGGGCAUCAGCAUGCUAAUUGU | 1100 | NM_000185.4 |
| SEQ ID NO: 21 | CAAAAAAGCAUGACAAACAGAAC | 1193 | NM_000185.4 |
| SEQ ID NO: 22 | GAGAGUAUUACUUUGCUGAGGCC | 771 | NM_000185.4 |
| SEQ ID NO: 23 | UUUCCUUAGGUCUGAAGGGAGAG | 543 | NM_000185.4 |
| SEQ ID NO: 24 | GCCAUCGACCUGUUCAAGCACCA | 1346 | NM_000185.4 |
| SEQ ID NO: 25 | GCUCACCAAGGGCCUCAUAAAAG | 853 | NM_000185.4 |
| SEQ ID NO: 26 | UGAAUAAAUUCCCAGUGGAAAUG | 948 | NM_000185.4 |
| SEQ ID NO: 27 | UGGCAAAAAAGCAUGACAAACAG | 1190 | NM_000185.4 |
| SEQ ID NO: 28 | ACUUCCGGCUGAAUGAGAGAGAG | 984 | NM_000185.4 |
| SEQ ID NO: 29 | AAGCAUGACAAACAGAACUCGAG | 1198 | NM_000185.4 |
| SEQ ID NO: 30 | GCCUGCUCUUCAUGGGAAGAGUG | 1506 | NM_000185.4 |
| SEQ ID NO: 31 | AGAGAGUAUUACUUUGCUGAGGC | 770 | NM_000185.4 |
| SEQ ID NO: 32 | CUCUUCAGGAGGAAUUUUGGGUA | 674 | NM_000185.4 |
| SEQ ID NO: 33 | AAAAGCAUGACAAACAGAACUCG | 1196 | NM_000185.4 |
| SEQ ID NO: 34 | GGGUGAAUAAAUUCCCAGUGGAA | 945 | NM_000185.4 |
| SEQ ID NO: 35 | CCGGAUCCAGCGUCUUAACAUCC | 412 | NM_000185.4 |
| SEQ ID NO: 36 | AUGACAAACAGAACUCGAGAAGU | 1202 | NM_000185.4 |
| SEQ ID NO: 37 | GCAUCUCAGACCAAAGGAUCGCC | 1326 | NM_000185.4 |
| SEQ ID NO: 38 | CACAACUUCCGGCUGAAUGAGAG | 980 | NM_000185.4 |
| SEQ ID NO: 39 | UCUUCAGGAGGAAUUUUGGGUAC | 675 | NM_000185.4 |
| SEQ ID NO: 40 | AAGAGAGUAUUACUUUGCUGAGG | 769 | NM_000185.4 |
| SEQ ID NO: 41 | AACCACAACUUCCGGCUGAAUGA | 977 | NM_000185.4 |
| SEQ ID NO: 42 | GCCGGAUCCAGCGUCUUAACAUC | 411 | NM_000185.4 |
| SEQ ID NO: 43 | AUUCUCAACUGCAUCUACUUCAA | 914 | NM_000185.4 |
| SEQ ID NO: 44 | AGGCAUCUCAGACCAAAGGAUCG | 1324 | NM_000185.4 |
| SEQ ID NO: 45 | AAAAAGCAUGACAAACAGAACUC | 1195 | NM_000185.4 |
| SEQ ID NO: 46 | GCUCUGGAGAAUAUAGACCCUGC | 878 | NM_000185.4 |
| SEQ ID NO: 47 | UAAGAGAGUAUUACUUUGCUGAG | 768 | NM_000185.4 |
| SEQ ID NO: 48 | UUCCGGCUGAAUGAGAGAGAGGU | 986 | NM_000185.4 |
| SEQ ID NO: 49 | AUCCUGGGUGAAUAAAUUCCCAG | 940 | NM_000185.4 |
| SEQ ID NO: 50 | UCCUUAGGUCUGAAGGGAGAGAC | 545 | NM_000185.4 |
| SEQ ID NO: 51 | AACUUCCGGCUGAAUGAGAGAGA | 983 | NM_000185.4 |
| SEQ ID NO: 52 | AAUAAAUUCCCAGUGGAAAUGAC | 950 | NM_000185.4 |
| SEQ ID NO: 53 | ACAACCACAACUUCCGGCUGAAU | 975 | NM_000185.4 |
| SEQ ID NO: 54 | CUGGAGAAUAUAGACCCUGCUAC | 881 | NM_000185.4 |
| SEQ ID NO: 55 | CGCCAUCGACCUGUUCAAGCACC | 1345 | NM_000185.4 |
| SEQ ID NO: 56 | GAUUCUCAACUGCAUCUACUUCA | 913 | NM_000185.4 |
| SEQ ID NO: 57 | GAUCCAGCGUCUUAACAUCCUCA | 415 | NM_000185.4 |
| SEQ ID NO: 58 | GCAGGCAUCUCAGACCAAAGGAU | 1322 | NM_000185.4 |
| SEQ ID NO: 59 | AUGCCGCUGUCCACCCAAGUCCG | 1430 | NM_000185.4 |
| SEQ ID NO: 60 | GCAUGACAAACAGAACUCGAGAA | 1200 | NM_000185.4 |
| SEQ ID NO: 61 | GUGGGGUUCAUGCCGCUGUCCAC | 1421 | NM_000185.4 |
| SEQ ID NO: 62 | CGGAUCCAGCGUCUUAACAUCCU | 413 | NM_000185.4 |
| SEQ ID NO: 63 | CACCCAAGUCCGCUUCACUGUCG | 1441 | NM_000185.4 |
| SEQ ID NO: 64 | CAACUUCCGGCUGAAUGAGAGAG | 982 | NM_000185.4 |
| SEQ ID NO: 65 | AGUAUUACUUUGCUGAGGCCCAG | 774 | NM_000185.4 |
| SEQ ID NO: 66 | CUCUGGAGAAUAUAGACCCUGCU | 879 | NM_000185.4 |
| SEQ ID NO: 67 | GAUGAUUCUCAACUGCAUCUACU | 910 | NM_000185.4 |
| SEQ ID NO: 68 | CAUGCCGCUGUCCACCCAAGUCC | 1429 | NM_000185.4 |
| SEQ ID NO: 69 | UUCUCAACUGCAUCUACUUCAAA | 915 | NM_000185.4 |
| SEQ ID NO: 70 | CAACCACAACUUCCGGCUGAAUG | 976 | NM_000185.4 |
| SEQ ID NO: 71 | CACGGUGGGGUUCAUGCCGCUGU | 1417 | NM_000185.4 |
| SEQ ID NO: 72 | CUUCCGGCUGAAUGAGAGAGAGG | 985 | NM_000185.4 |
| SEQ ID NO: 73 | CCUCUUCAGGAGGAAUUUUGGGU | 673 | NM_000185.4 |
| SEQ ID NO: 74 | GAGUAUUACUUUGCUGAGGCCCA | 773 | NM_000185.4 |
| SEQ ID NO: 75 | AUGAUUCUCAACUGCAUCUACUU | 911 | NM_000185.4 |
| SEQ ID NO: 76 | GGCAUCUCAGACCAAAGGAUCGC | 1325 | NM_000185.4 |
| SEQ ID NO: 77 | CAGGCAUCUCAGACCAAAGGAUC | 1323 | NM_000185.4 |
| SEQ ID NO: 78 | UCUCAACUGCAUCUACUUCAAAG | 916 | NM_000185.4 |
| SEQ ID NO: 79 | CCCAAGUCCGCUUCACUGUCGAC | 1443 | NM_000185.4 |
| SEQ ID NO: 80 | GGGGGGCAUCAGCAUGCUAAUUG | 1099 | NM_000185.4 |
| SEQ ID NO: 81 | AGAGUAUUACUUUGCUGAGGCCC | 772 | NM_000185.4 |
| SEQ ID NO: 82 | ACGGUGGGGUUCAUGCCGCUGUC | 1418 | NM_000185.4 |
| SEQ ID NO: 83 | GCACCAGCUGCCUGCUCUUCAUG | 1497 | NM_000185.4 |
| SEQ ID NO: 84 | CCUGGGUGAAUAAAUUCCCAGUG | 942 | NM_000185.4 |
| SEQ ID NO: 85 | GCAAAAAAGCAUGACAAACAGAA | 1192 | NM_000185.4 |
| SEQ ID NO: 86 | ACCCAAGUCCGCUUCACUGUCGA | 1442 | NM_000185.4 |
| SEQ ID NO: 87 | GCAAGAGCCGGAUCCAGCGUCUU | 405 | NM_000185.4 |
| SEQ ID NO: 88 | AAAAAAGCAUGACAAACAGAACU | 1194 | NM_000185.4 |
| SEQ ID NO: 89 | GUUCAUGCCGCUGUCCACCCAAG | 1426 | NM_000185.4 |
| SEQ ID NO: 90 | AAAGCAUGACAAACAGAACUCGA | 1197 | NM_000185.4 |
| SEQ ID NO: 91 | GACACACAACCACAACUUCCGGC | 970 | NM_000185.4 |
| SEQ ID NO: 92 | CACACAACCACAACUUCCGGCUG | 972 | NM_000185.4 |
| SEQ ID NO: 93 | CAAGAGCCGGAUCCAGCGUCUUA | 406 | NM_000185.4 |
| SEQ ID NO: 94 | ACACAACCACAACUUCCGGCUGA | 973 | NM_000185.4 |
| SEQ ID NO: 95 | CCAUCGACCUGUUCAAGCACCAA | 1347 | NM_000185.4 |
| SEQ ID NO: 96 | CGGGUGGUGGAGAGAUGGCAAAA | 1175 | NM_000185.4 |
| SEQ ID NO: 97 | CCACCCAAGUCCGCUUCACUGUC | 1440 | NM_000185.4 |
| SEQ ID NO: 98 | AGCAUGACAAACAGAACUCGAGA | 1199 | NM_000185.4 |
| SEQ ID NO: 99 | GAGCCGGAUCCAGCGUCUUAACA | 409 | NM_000185.4 |
| SEQ ID NO: | UGGCAAGAGCCGGAUCCAGCGUC | 403 | NM_000185.4 |
| 100 | | | |
| SEQ ID NO: | AUGGCAAGAGCCGGAUCCAGCGU | 402 | NM_000185.4 |
| 101 | | | |
| SEQ ID NO: | GGUGGGGUUCAUGCCGCUGUCCA | 1420 | NM_000185.4 |
| 102 | | | |
| SEQ ID NO: | UUUGCCUUCAUCCACAAGGAUUU | 991 | NM_016186.3 |
| 103 | | | |
| SEQ ID NO: | GCUGCGAAAGAUCUCCAUGAGGC | 756 | NM_016186.3 |
| 104 | | | |
| SEQ ID NO: | AUGCUGGUGGUCCUCAUGGAGAA | 1390 | NM_016186.3 |
| 105 | | | |
| SEQ ID NO: | CUGCGAAAGAUCUCCAUGAGGCA | 757 | NM_016186.3 |
| 106 | | | |
| SEQ ID NO: | AAGUAUGAGAUGCAUGAGCUGCU | 1531 | NM_016186.3 |
| 107 | | | |
| SEQ ID NO: | CUGUUUGAUGAGAUUAAUCCUGA | 1156 | NM_016186.3 |
| 108 | | | |
| SEQ ID NO: | GAUGAGAUUAAUCCUGAAACCAA | 1162 | NM_016186.3 |
| 109 | | | |
| SEQ ID NO: | UUUGAUGAGAUUAAUCCUGAAAC | 1159 | NM_016186.3 |
| 110 | | | |
| SEQ ID NO: | UGAUGAGAUUAAUCCUGAAACCA | 1161 | NM_016186.3 |
| 111 | | | |
| SEQ ID NO: | UUGAUGAGAUUAAUCCUGAAACC | 1160 | NM_016186.3 |
| 112 | | | |
| SEQ ID NO: | AACUGUUUGAUGAGAUUAAUCCU | 1154 | NM_016186.3 |
| 113 | | | |
| SEQ ID NO: | AGUUUUGCCUUCAUCCACAAGGA | 988 | NM_016186.3 |
| 114 | | | |
| SEQ ID NO: | UGCGAAAGAUCUCCAUGAGGCAC | 758 | NM_016186.3 |
| 115 | | | |
| SEQ ID NO: | CAUGCUGGUGGUCCUCAUGGAGA | 1389 | NM_016186.3 |
| 116 | | | |
| SEQ ID NO: | UGCCUUCAUCCACAAGGAUUUUG | 993 | NM_016186.3 |
| 117 | | | |
| SEQ ID NO: | GCGAAAGAUCUCCAUGAGGCACG | 759 | NM_016186.3 |
| 118 | | | |
| SEQ ID NO: | CCUUCAUCCACAAGGAUUUUGAU | 995 | NM_016186.3 |
| 119 | | | |
| SEQ ID NO: | UGUUUGAUGAGAUUAAUCCUGAA | 1157 | NM_016186.3 |
| 120 | | | |
| SEQ ID NO: | UUUUGCCUUCAUCCACAAGGAUU | 990 | NM_016186.3 |
| 121 | | | |
| SEQ ID NO: | AAGAUCUCCAUGAGGCACGAUGG | 763 | NM_016186.3 |
| 122 | | | |
| SEQ ID NO: | ACCAUGCUGGUGGUCCUCAUGGA | 1387 | NM_016186.3 |
| 123 | | | |
| SEQ ID NO: | GUUUUGCCUUCAUCCACAAGGAU | 989 | NM_016186.3 |
| 124 | | | |
| SEQ ID NO: | UUGCCUUCAUCCACAAGGAUUUU | 992 | NM_016186.3 |
| 125 | | | |
| SEQ ID NO: | CCUACCAAGGAAAUGCCACCAUG | 1370 | NM_016186.3 |
| 126 | | | |
| SEQ ID NO: | GUUUGAUGAGAUUAAUCCUGAAA | 1158 | NM_016186.3 |
| 127 | | | |
| SEQ ID NO: | GCCUUCAUCCACAAGGAUUUUGA | 994 | NM_016186.3 |
| 128 | | | |
| SEQ ID NO: | CGAAAGAUCUCCAUGAGGCACGA | 760 | NM_016186.3 |
| 129 | | | |
| SEQ ID NO: | ACUGUUUGAUGAGAUUAAUCCUG | 1155 | NM_016186.3 |
| 130 | | | |
| SEQ ID NO: | CCAUGCUGGUGGUCCUCAUGGAG | 1388 | NM_016186.3 |
| 131 | | | |
| SEQ ID NO: | GAGUUUUGCCUUCAUCCACAAGG | 987 | NM_016186.3 |
| 132 | | | |
| SEQ ID NO: | UGCCACCAUGCUGGUGGUCCUCA | 1383 | NM_016186.3 |
| 133 | | | |
| SEQ ID NO: | GCCACCAUGCUGGUGGUCCUCAU | 1384 | NM_016186.3 |
| 134 | | | |
| SEQ ID NO: | GAAAGAUCUCCAUGAGGCACGAU | 761 | NM_016186.3 |
| 135 | | | |
| SEQ ID NO: | GGAGUUUUGCCUUCAUCCACAAG | 986 | NM_016186.3 |
| 136 | | | |
| SEQ ID NO: | CCACCAUGCUGGUGGUCCUCAUG | 1385 | NM_016186.3 |
| 137 | | | |
| SEQ ID NO: | AAAGAUCUCCAUGAGGCACGAUG | 762 | NM_016186.3 |
| 138 | | | |
| SEQ ID NO: | CACCAUGCUGGUGGUCCUCAUGG | 1386 | NM_016186.3 |
| 139 | | | |
| SEQ ID NO: | UUUGCCUCCACCUUUGACAAGAA | 1321 | NM_016186.3 |
| 140 | | | |
| SEQ ID NO: | UGCCUCCACCUUUGACAAGAAUU | 1323 | NM_016186.3 |
| 141 | | | |
| SEQ ID NO: | ACCAUUAAGGUGCCCAUGAUGUA | 1285 | NM_016186.3 |
| 142 | | | |
| SEQ ID NO: | AACUGCCCUACCAAGGAAAUGCC | 1364 | NM_016186.3 |
| 143 | | | |
| SEQ ID NO: | CUCAAACUGCCCUACCAAGGAAA | 1360 | NM_016186.3 |
| 144 | | | |
| SEQ ID NO: | CCUCAAACUGCCCUACCAAGGAA | 1359 | NM_016186.3 |
| 145 | | | |
| SEQ ID NO: | GCCUCCACCUUUGACAAGAAUUU | 1324 | NM_016186.3 |
| 146 | | | |
| SEQ ID NO: | GUCGACACUUUCCACCUGGACAA | 1255 | NM_016186.3 |
| 147 | | | |
| SEQ ID NO: | ACACUUUCCACCUGGACAAGUAC | 1259 | NM_016186.3 |
| 148 | | | |
| SEQ ID NO: | UCAAACUGCCCUACCAAGGAAAU | 1361 | NM_016186.3 |
| 149 | | | |
| SEQ ID NO: | AAACUGCCCUACCAAGGAAAUGC | 1363 | NM_016186.3 |
| 150 | | | |
| SEQ ID NO: | GACACUUUCCACCUGGACAAGUA | 1258 | NM_016186.3 |
| 151 | | | |
| SEQ ID NO: | GACCAUUAAGGUGCCCAUGAUGU | 1284 | NM_016186.3 |
| 152 | | | |
| SEQ ID NO: | GUUUGCCUCCACCUUUGACAAGA | 1320 | NM_016186.3 |
| 153 | | | |
| SEQ ID NO: | ACUUUCCACCUGGACAAGUACAA | 1261 | NM_016186.3 |
| 154 | | | |
| SEQ ID NO: | UGUCCUCAAACUGCCCUACCAAG | 1356 | NM_016186.3 |
| 155 | | | |
| SEQ ID NO: | CAAACUGCCCUACCAAGGAAAUG | 1362 | NM_016186.3 |
| 156 | | | |
| SEQ ID NO: | GUCCUCAAACUGCCCUACCAAGG | 1357 | NM_016186.3 |
| 157 | | | |
| SEQ ID NO: | AUGUCCUCAAACUGCCCUACCAA | 1355 | NM_016186.3 |
| 158 | | | |
| SEQ ID NO: | UCGACACUUUCCACCUGGACAAG | 1256 | NM_016186.3 |
| 159 | | | |
| SEQ ID NO: | AGUUUGCCUCCACCUUUGACAAG | 1319 | NM_016186.3 |
| 160 | | | |
| SEQ ID NO: | CGACACUUUCCACCUGGACAAGU | 1257 | NM_016186.3 |
| 161 | | | |
| SEQ ID NO: | CUUUCCACCUGGACAAGUACAAG | 1262 | NM_016186.3 |
| 162 | | | |
| SEQ ID NO: | CACUUUCCACCUGGACAAGUACA | 1260 | NM_016186.3 |
| 163 | | | |
| SEQ ID NO: | UCCUCAAACUGCCCUACCAAGGA | 1358 | NM_016186.3 |
| 164 | | | |
| SEQ ID NO: | GAUUACAUCUUGUUCAAAGGGAA | 1198 | NM_016186.3 |
| 165 | | | |
| SEQ ID NO: | AAUGCCACCAUGCUGGUGGUCCU | 1381 | NM_016186.3 |
| 166 | | | |
| SEQ ID NO: | UUUAUCCAAGAGGUAUUUUGAUA | 1038 | NM_016186.3 |
| 167 | | | |
| SEQ ID NO: | GGAAAUGCCACCAUGCUGGUGGU | 1378 | NM_016186.3 |
| 168 | | | |
| SEQ ID NO: | GGAUUACAUCUUGUUCAAAGGGA | 1197 | NM_016186.3 |
| 169 | | | |
| SEQ ID NO: | AUCUCCAUGAGGCACGAUGGCAA | 766 | NM_016186.3 |
| 170 | | | |
| SEQ ID NO: | AUUCCAUGCCUCCUGUCAUCAAA | 1721 | NM_016186.3 |
| 171 | | | |
| SEQ ID NO: | UUAUUCCAUGCCUCCUGUCAUCA | 1719 | NM_016186.3 |
| 172 | | | |
| SEQ ID NO: | ACCAAGGAAAUGCCACCAUGCUG | 1373 | NM_016186.3 |
| 173 | | | |
| SEQ ID NO: | GCUGGUGGUCCUCAUGGAGAAAA | 1392 | NM_016186.3 |
| 174 | | | |
| SEQ ID NO: | ACAUCUUGUUCAAAGGGAAAUGG | 1202 | NM_016186.3 |
| 175 | | | |
| SEQ ID NO: | CCAAGGAAAUGCCACCAUGCUGG | 1374 | NM_016186.3 |
| 176 | | | |
| SEQ ID NO: | UUGCCUCCACCUUUGACAAGAAU | 1322 | NM_016186.3 |
| 177 | | | |
| SEQ ID NO: | GGGAGUUUUGCCUUCAUCCACAA | 985 | NM_016186.3 |
| 178 | | | |
| SEQ ID NO: | CUGCUGCGAAAGAUCUCCAUGAG | 754 | NM_016186.3 |
| 179 | | | |
| SEQ ID NO: | CAAGUUUGCCUCCACCUUUGACA | 1317 | NM_016186.3 |
| 180 | | | |
| SEQ ID NO: | AAGUUUGCCUCCACCUUUGACAA | 1318 | NM_016186.3 |
| 181 | | | |
| SEQ ID NO: | UACAUCUUGUUCAAAGGGAAAUG | 1201 | NM_016186.3 |
| 182 | | | |
| SEQ ID NO: | GGGGAGUUUUGCCUUCAUCCACA | 984 | NM_016186.3 |
| 183 | | | |
| SEQ ID NO: | UGCUGCGAAAGAUCUCCAUGAGG | 755 | NM_016186.3 |
| 184 | | | |
| SEQ ID NO: | UUCCAUGCCUCCUGUCAUCAAAG | 1722 | NM_016186.3 |
| 185 | | | |
| SEQ ID NO: | UCUGUUUCUGGGCAGGGUGGUGA | 1794 | NM_016186.3 |
| 186 | | | |
| SEQ ID NO: | CAGCCUGCUGCGAAAGAUCUCCA | 750 | NM_016186.3 |
| 187 | | | |
| SEQ ID NO: | CAAACUGUUUGAUGAGAUUAAUC | 1152 | NM_016186.3 |
| 188 | | | |
| SEQ ID NO: | CCAUGCCUCCUGUCAUCAAAGUG | 1724 | NM_016186.3 |
| 189 | | | |
| SEQ ID NO: | GAAGUAUGAGAUGCAUGAGCUGC | 1530 | NM_016186.3 |
| 190 | | | |
| SEQ ID NO: | AGAAGUAUGAGAUGCAUGAGCUG | 1529 | NM_016186.3 |
| 191 | | | |
| SEQ ID NO: | CAUGUCCUCAAACUGCCCUACCA | 1354 | NM_016186.3 |
| 192 | | | |
| SEQ ID NO: | AAACUGUUUGAUGAGAUUAAUCC | 1153 | NM_016186.3 |
| 193 | | | |
| SEQ ID NO: | AAGACCAUUAAGGUGCCCAUGAU | 1282 | NM_016186.3 |
| 194 | | | |
| SEQ ID NO: | AGACCAUUAAGGUGCCCAUGAUG | 1283 | NM_016186.3 |
| 195 | | | |
| SEQ ID NO: | CUGUUUCUGGGCAGGGUGGUGAA | 1795 | NM_016186.3 |
| 196 | | | |
| SEQ ID NO: | GCUUCUGUUUCUGGGCAGGGUGG | 1791 | NM_016186.3 |
| 197 | | | |
| SEQ ID NO: | AGUUUUCUUUCCGAAGUUCAAGC | 1500 | NM_016186.3 |
| 198 | | | |
| SEQ ID NO: | GUUUUCUUUCCGAAGUUCAAGCU | 1501 | NM_016186.3 |
| 199 | | | |
| SEQ ID NO: | CUUCUGUUUCUGGGCAGGGUGGU | 1792 | NM_016186.3 |
| 200 | | | |
| SEQ ID NO: | UCAUGUCCUCAAACUGCCCUACC | 1353 | NM_016186.3 |
| 201 | | | |
| SEQ ID NO: | AGUCGACACUUUCCACCUGGACA | 1254 | NM_016186.3 |
| 202 | | | |

Table 23 provides the unmodified first (antisense) and corresponding unmodified second (sense) strand sequences for siRNA oligonucleosides (targeting HCII and ZPI) according to the present invention, together with the corresponding positions in the overall gene sequence of SEQ ID NOs: 1 and 2 as follows.

TABLE 23

| | | | Second (Sense) | | |
|---|---|---|---|---|---|
| | | | Strand Base | | |
| | First (Antisense) | | Sequence | Corre- | |
| | Strand Base Sequence | | 5′ → 3′ | sponding | |
| | 5′ → 3′ | | (Shown as an | positions | |
| | (Shown as an | | Unmodified | on | Reference |
| SEQ ID | Unmodified | SEQ ID | Nucleoside | reference | sequence |
| NO (AS) | Nucleoside Sequence) | NO (SS) | Sequence) | sequence | ID |
| SEQ ID | UUUCCACUGGGAA | SEQ ID | UGAAUAAAUUC | 946-967 | NM_000185.4 |
| NO: 203 | UUUAUUCACC | NO: 403 | CCAGUGGAAA | | |
| SEQ ID | GAUCCUUUGAAGU | SEQ ID | CUGCAUCUACUU | 920-941 | NM_000185.4 |
| NO: 204 | AGAUGCAGUU | NO: 404 | CAAAGGAUC | | |
| SEQ ID | AUCCUUUGAAGUA | SEQ ID | ACUGCAUCUACU | 919-940 | NM_000185.4 |
| NO: 205 | GAUGCAGUUG | NO: 405 | UCAAAGGAU | | |
| SEQ ID | ACUUGUUCAUGGG | SEQ ID | GGGAGAGACCCA | 557-578 | NM_000185.4 |
| NO: 206 | UCUCUCCCUU | NO: 406 | UGAACAAGU | | |
| SEQ ID | UCCACUGGGAAUU | SEQ ID | GGUGAAUAAAU | 944-965 | NM_000185.4 |
| NO: 207 | UAUUCACCCA | NO: 407 | UCCCAGUGGA | | |
| SEQ ID | UGUACCCAAAAUU | SEQ ID | UCAGGAGGAAU | 676-697 | NM_000185.4 |
| NO: 208 | CCUCCUGAAG | NO: 408 | UUUGGGUACA | | |
| SEQ ID | UUCCCAUGAAGAG | SEQ ID | GCUGCCUGCUCU | 1501-1522 | NM_000185.4 |
| NO: 209 | CAGGCAGCUG | NO: 409 | UCAUGGGAA | | |
| SEQ ID | UCUUUUAUGAGGC | SEQ ID | CACCAAGGGCCU | 854-875 | NM_000185.4 |
| NO: 210 | CCUUGGUGAG | NO: 410 | CAUAAAAGA | | |
| SEQ ID | AACUGCUUCUGGA | SEQ ID | CCUUUAUAUCCA | 716-737 | NM_000185.4 |
| NO: 211 | UAUAAAGGUC | NO: 411 | GAAGCAGUU | | |
| SEQ ID | CCUUUGAAGUAGA | SEQ ID | CAACUGCAUCUA | 917-938 | NM_000185.4 |
| NO: 212 | UGCAGUUGAG | NO: 412 | CUUCAAAGG | | |
| SEQ ID | UUAAGACGCUGGA | SEQ ID | GAGCCGGAUCCA | 407-428 | NM_000185.4 |
| NO: 213 | UCCGGCUCUU | NO: 413 | GCGUCUUAA | | |
| SEQ ID | GAGGAUGUUAAG | SEQ ID | AUCCAGCGUCUU | 414-435 | NM_000185.4 |
| NO: 214 | ACGCUGGAUCC | NO: 414 | AACAUCCUC | | |
| SEQ ID | UCUGUUUGUCAUG | SEQ ID | CAAAAAAGCAU | 1191-1212 | NM_000185.4 |
| NO: 215 | CUUUUUUGCC | NO: 415 | GACAAACAGA | | |
| SEQ ID | GUGUACCCAAAAU | SEQ ID | CAGGAGGAAUU | 677-698 | NM_000185.4 |
| NO: 216 | UCCUCCUGAA | NO: 416 | UUGGGUACAC | | |
| SEQ ID | UUCAGCCGGAAGU | SEQ ID | CAACCACAACUU | 974-995 | NM_000185.4 |
| NO: 217 | UGUGGUUGUG | NO: 417 | CCGGCUGAA | | |
| SEQ ID | UGUUUGUCAUGCU | SEQ ID | GGCAAAAAAGC | 1189-1210 | NM_000185.4 |
| NO: 218 | UUUUUGCCAU | NO: 418 | AUGACAAACA | | |
| SEQ ID | AUUUCCACUGGGA | SEQ ID | GAAUAAAUUCCC | 947-968 | NM_000185.4 |
| NO: 219 | AUUUAUUCAC | NO: 419 | AGUGGAAAU | | |
| SEQ ID | ACAAUUAGCAUGC | SEQ ID | GGGCAUCAGCAU | 1100-1121 | NM_000185.4 |
| NO: 220 | UGAUGCCCCC | NO: 420 | GCUAAUUGU | | |
| SEQ ID | GUUCUGUUUGUCA | SEQ ID | AAAAAGCAUGA | 1193-1214 | NM_000185.4 |
| NO: 221 | UGCUUUUUUG | NO: 421 | CAAACAGAAC | | |
| SEQ ID | GGCCUCAGCAAAG | SEQ ID | GAGUAUUACUU | 771-792 | NM_000185.4 |
| NO: 222 | UAAUACUCUC | NO: 422 | UGCUGAGGCC | | |
| SEQ ID | CUCUCCCUUCAGA | SEQ ID | UCCUUAGGUCUG | 543-564 | NM_000185.4 |
| NO: 223 | CCUAAGGAAA | NO: 423 | AAGGGAGAG | | |
| SEQ ID | UGGUGCUUGAACA | SEQ ID | CAUCGACCUGUU | 1346-1367 | NM_000185.4 |
| NO: 224 | GGUCGAUGGC | NO: 424 | CAAGCACCA | | |
| SEQ ID | CUUUUAUGAGGCC | SEQ ID | UCACCAAGGGCC | 853-874 | NM_000185.4 |
| NO: 225 | CUUGGUGAGC | NO: 425 | UCAUAAAAG | | |
| SEQ ID | CAUUUCCACUGGG | SEQ ID | AAUAAAUUCCCA | 948-969 | NM_000185.4 |
| NO: 226 | AAUUUAUUCA | NO: 426 | GUGGAAAUG | | |
| SEQ ID | CUGUUUGUCAUGC | SEQ ID | GCAAAAAAGCA | 1190-1211 | NM_000185.4 |
| NO: 227 | UUUUUUGCCA | NO: 427 | UGACAAACAG | | |
| SEQ ID | CUCUCUCUCAUUC | SEQ ID | UUCCGGCUGAAU | 984-1005 | NM_000185.4 |
| NO: 228 | AGCCGGAAGU | NO: 428 | GAGAGAGAG | | |
| SEQ ID | CUCGAGUUCUGUU | SEQ ID | GCAUGACAAACA | 1198-1219 | NM_000185.4 |
| NO: 229 | UGUCAUGCUU | NO: 429 | GAACUCGAG | | |
| SEQ ID | CACUCUUCCCAUG | SEQ ID | CUGCUCUUCAUG | 1506-1527 | NM_000185.4 |
| NO: 230 | AAGAGCAGGC | NO: 430 | GGAAGAGUG | | |
| SEQ ID | GCCUCAGCAAAGU | SEQ ID | AGAGUAUUACU | 770-791 | NM_000185.4 |
| NO: 231 | AAUACUCUCU | NO: 431 | UUGCUGAGGC | | |
| SEQ ID | UACCCAAAAUUCC | SEQ ID | CUUCAGGAGGA | 674-695 | NM_000185.4 |
| NO: 232 | UCCUGAAGAG | NO: 432 | AUUUUGGGUA | | |
| SEQ ID | CGAGUUCUGUUUG | SEQ ID | AAGCAUGACAA | 1196-1217 | NM_000185.4 |
| NO: 233 | UCAUGCUUUU | NO: 433 | ACAGAACUCG | | |
| SEQ ID | UUCCACUGGGAAU | SEQ ID | GUGAAUAAAUU | 945-966 | NM_000185.4 |
| NO: 234 | UUAUUCACCC | NO: 434 | CCCAGUGGAA | | |
| SEQ ID | GGAUGUUAAGAC | SEQ ID | GGAUCCAGCGUC | 412-433 | NM_000185.4 |
| NO: 235 | GCUGGAUCCGG | NO: 435 | UUAACAUCC | | |
| SEQ ID | ACUUCUCGAGUUC | SEQ ID | GACAAACAGAAC | 1202-1223 | NM_000185.4 |
| NO: 236 | UGUUUGUCAU | NO: 436 | UCGAGAAGU | | |
| SEQ ID | GGCGAUCCUUUGG | SEQ ID | AUCUCAGACCAA | 1326-1347 | NM_000185.4 |
| NO: 237 | UCUGAGAUGC | NO: 437 | AGGAUCGCC | | |
| SEQ ID | CUCUCAUUCAGCC | SEQ ID | CAACUUCCGGCU | 980-1001 | NM_000185.4 |
| NO: 238 | GGAAGUUGUG | NO: 438 | GAAUGAGAG | | |
| SEQ ID | GUACCCAAAAUUC | SEQ ID | UUCAGGAGGAA | 675-696 | NM_000185.4 |
| NO: 239 | CUCCUGAAGA | NO: 439 | UUUUGGGUAC | | |
| SEQ ID | CCUCAGCAAAGUA | SEQ ID | GAGAGUAUUAC | 769-790 | NM_000185.4 |
| NO: 240 | AUACUCUCUU | NO: 440 | UUUGCUGAGG | | |
| SEQ ID | UCAUUCAGCCGGA | SEQ ID | CCACAACUUCCG | 977-998 | NM_000185.4 |
| NO: 241 | AGUUGUGGUU | NO: 441 | GCUGAAUGA | | |
| SEQ ID | GAUGUUAAGACGC | SEQ ID | CGGAUCCAGCGU | 411-432 | NM_000185.4 |
| NO: 242 | UGGAUCCGGC | NO: 442 | CUUAACAUC | | |
| SEQ ID | UUGAAGUAGAUG | SEQ ID | UCUCAACUGCAU | 914-935 | NM_000185.4 |
| NO: 243 | CAGUUGAGAAU | NO: 443 | CUACUUCAA | | |
| SEQ ID | CGAUCCUUUGGUC | SEQ ID | GCAUCUCAGACC | 1324-1345 | NM_000185.4 |
| NO: 244 | UGAGAUGCCU | NO: 444 | AAAGGAUCG | | |
| SEQ ID | GAGUUCUGUUUG | SEQ ID | AAAGCAUGACA | 1195-1216 | NM_000185.4 |
| NO: 245 | UCAUGCUUUUU | NO: 445 | AACAGAACUC | | |
| SEQ ID | GCAGGGUCUAUAU | SEQ ID | UCUGGAGAAUA | 878-899 | NM_000185.4 |
| NO: 246 | UCUCCAGAGC | NO: 446 | UAGACCCUGC | | |
| SEQ ID | CUCAGCAAAGUAA | SEQ ID | AGAGAGUAUUA | 768-789 | NM_000185.4 |
| NO: 247 | UACUCUCUUA | NO: 447 | CUUUGCUGAG | | |
| SEQ ID | ACCUCUCUCUCAU | SEQ ID | CCGGCUGAAUGA | 9 86-1007 | NM_000185.4 |
| NO: 248 | UCAGCCGGAA | NO: 448 | GAGAGAGGU | | |
| SEQ ID | CUGGGAAUUUAU | SEQ ID | CCUGGGUGAAU | 940-961 | NM_000185.4 |
| NO: 249 | UCACCCAGGAU | NO: 449 | AAAUUCCCAG | | |
| SEQ ID | GUCUCUCCCUUCA | SEQ ID | CUUAGGUCUGA | 545-566 | NM_000185.4 |
| NO: 250 | GACCUAAGGA | NO: 450 | AGGGAGAGAC | | |
| SEQ ID | UCUCUCUCAUUCA | SEQ ID | CUUCCGGCUGAA | 983-1004 | NM_000185.4 |
| NO: 251 | GCCGGAAGUU | NO: 451 | UGAGAGAGA | | |
| SEQ ID | GUCAUUUCCACUG | SEQ ID | UAAAUUCCCAGU | 950-971 | NM_000185.4 |
| NO: 252 | GGAAUUUAUU | NO: 452 | GGAAAUGAC | | |
| SEQ ID | AUUCAGCCGGAAG | SEQ ID | AACCACAACUUC | 975-996 | NM_000185.4 |
| NO: 253 | UUGUGGUUGU | NO: 453 | CGGCUGAAU | | |
| SEQ ID | GUAGCAGGGUCUA | SEQ ID | GGAGAAUAUAG | 881-902 | NM_000185.4 |
| NO: 254 | UAUUCUCCAG | NO: 454 | ACCCUGCUAC | | |
| SEQ ID | GGUGCUUGAACAG | SEQ ID | CCAUCGACCUGU | 1345-1366 | NM_000185.4 |
| NO: 255 | GUCGAUGGCG | NO: 455 | UCAAGCACC | | |
| SEQ ID | UGAAGUAGAUGC | SEQ ID | UUCUCAACUGCA | 913-934 | NM_000185.4 |
| NO: 256 | AGUUGAGAAUC | NO: 456 | UCUACUUCA | | |
| SEQ ID | UGAGGAUGUUAA | SEQ ID | UCCAGCGUCUUA | 415-436 | NM_000185.4 |
| NO: 257 | GACGCUGGAUC | NO: 457 | ACAUCCUCA | | |
| SEQ ID | AUCCUUUGGUCUG | SEQ ID | AGGCAUCUCAGA | 1322-1343 | NM_000185.4 |
| NO: 258 | AGAUGCCUGC | NO: 458 | CCAAAGGAU | | |
| SEQ ID | CGGACUUGGGUGG | SEQ ID | GCCGCUGUCCAC | 1430-1451 | NM_000185.4 |
| NO: 259 | ACAGCGGCAU | NO: 459 | CCAAGUCCG | | |
| SEQ ID | UUCUCGAGUUCUG | SEQ ID | AUGACAAACAG | 1200-1221 | NM_000185.4 |
| NO: 260 | UUUGUCAUGC | NO: 460 | AACUCGAGAA | | |
| SEQ ID | GUGGACAGCGGCA | SEQ ID | GGGGUUCAUGCC | 1421-1442 | NM_000185.4 |
| NO: 261 | UGAACCCCAC | NO: 461 | GCUGUCCAC | | |
| SEQ ID | AGGAUGUUAAGA | SEQ ID | GAUCCAGCGUCU | 413-434 | NM_000185.4 |
| NO: 262 | CGCUGGAUCCG | NO: 462 | UAACAUCCU | | |
| SEQ ID | CGACAGUGAAGCG | SEQ ID | CCCAAGUCCGCU | 1441-1462 | NM_000185.4 |
| NO: 263 | GACUUGGGUG | NO: 463 | UCACUGUCG | | |
| SEQ ID | CUCUCUCAUUCAG | SEQ ID | ACUUCCGGCUGA | 982-1003 | NM_000185.4 |
| NO: 264 | CCGGAAGUUG | NO: 464 | AUGAGAGAG | | |
| SEQ ID | CUGGGCCUCAGCA | SEQ ID | UAUUACUUUGC | 774-795 | NM_000185.4 |
| NO: 265 | AAGUAAUACU | NO: 465 | UGAGGCCCAG | | |
| SEQ ID | AGCAGGGUCUAUA | SEQ ID | CUGGAGAAUAU | 879-900 | NM_000185.4 |
| NO: 266 | UUCUCCAGAG | NO: 466 | AGACCCUGCU | | |
| SEQ ID | AGUAGAUGCAGU | SEQ ID | UGAUUCUCAACU | 910-931 | NM_000185.4 |
| NO: 267 | UGAGAAUCAUC | NO: 467 | GCAUCUACU | | |
| SEQ ID | GGACUUGGGUGG | SEQ ID | UGCCGCUGUCCA | 1429-1450 | NM_000185.4 |
| NO: 268 | ACAGCGGCAUG | NO: 468 | CCCAAGUCC | | |
| SEQ ID | UUUGAAGUAGAU | SEQ ID | CUCAACUGCAUC | 915-936 | NM_000185.4 |
| NO: 269 | GCAGUUGAGAA | NO: 469 | UACUUCAAA | | |
| SEQ ID | CAUUCAGCCGGAA | SEQ ID | ACCACAACUUCC | 976-997 | NM_000185.4 |
| NO: 270 | GUUGUGGUUG | NO: 470 | GGCUGAAUG | | |
| SEQ ID | ACAGCGGCAUGAA | SEQ ID | CGGUGGGGUUC | 1417-1438 | NM_000185.4 |
| NO: 271 | CCCCACCGUG | NO: 471 | AUGCCGCUGU | | |
| SEQ ID | CCUCUCUCUCAUU | SEQ ID | UCCGGCUGAAUG | 985-1006 | NM_000185.4 |
| NO: 272 | CAGCCGGAAG | NO: 472 | AGAGAGAGG | | |
| SEQ ID | ACCCAAAAUUCCU | SEQ ID | UCUUCAGGAGG | 673-694 | NM_000185.4 |
| NO: 273 | CCUGAAGAGG | NO: 473 | AAUUUUGGGU | | |
| SEQ ID | UGGGCCUCAGCAA | SEQ ID | GUAUUACUUUG | 773-794 | NM_000185.4 |
| NO: 274 | AGUAAUACUC | NO: 474 | CUGAGGCCCA | | |
| SEQ ID | AAGUAGAUGCAG | SEQ ID | GAUUCUCAACUG | 911-932 | NM_000185.4 |
| NO: 275 | UUGAGAAUCAU | NO: 475 | CAUCUACUU | | |
| SEQ ID | GCGAUCCUUUGGU | SEQ ID | CAUCUCAGACCA | 1325-1346 | NM_000185.4 |
| NO: 276 | CUGAGAUGCC | NO: 476 | AAGGAUCGC | | |
| SEQ ID | GAUCCUUUGGUCU | SEQ ID | GGCAUCUCAGAC | 1323-1344 | NM_000185.4 |
| NO: 277 | GAGAUGCCUG | NO: 477 | CAAAGGAUC | | |
| SEQ ID | CUUUGAAGUAGA | SEQ ID | UCAACUGCAUCU | 916-937 | NM_000185.4 |
| NO: 278 | UGCAGUUGAGA | NO: 478 | ACUUCAAAG | | |
| SEQ ID | GUCGACAGUGAAG | SEQ ID | CAAGUCCGCUUC | 1443-1464 | NM_000185.4 |
| NO: 279 | CGGACUUGGG | NO: 479 | ACUGUCGAC | | |
| SEQ ID | CAAUUAGCAUGCU | SEQ ID | GGGGCAUCAGCA | 1099-1120 | NM_000185.4 |
| NO: 280 | GAUGCCCCCC | NO: 480 | UGCUAAUUG | | |
| SEQ ID | GGGCCUCAGCAAA | SEQ ID | AGUAUUACUUU | 772-793 | NM_000185.4 |
| NO: 281 | GUAAUACUCU | NO: 481 | GCUGAGGCCC | | |
| SEQ ID | GACAGCGGCAUGA | SEQ ID | GGUGGGGUUCA | 1418-1439 | NM_000185.4 |
| NO: 282 | ACCCCACCGU | NO: 482 | UGCCGCUGUC | | |
| SEQ ID | CAUGAAGAGCAGG | SEQ ID | ACCAGCUGCCUG | 1497-1518 | NM_000185.4 |
| NO: 283 | CAGCUGGUGC | NO: 483 | CUCUUCAUG | | |
| SEQ ID | CACUGGGAAUUUA | SEQ ID | UGGGUGAAUAA | 942-963 | NM_000185.4 |
| NO: 284 | UUCACCCAGG | NO: 484 | AUUCCCAGUG | | |
| SEQ ID | UUCUGUUUGUCAU | SEQ ID | AAAAAAGCAUG | 1192-1213 | NM_000185.4 |
| NO: 285 | GCUUUUUUGC | NO: 485 | ACAAACAGAA | | |
| SEQ ID | UCGACAGUGAAGC | SEQ ID | CCAAGUCCGCUU | 1442-1463 | NM_000185.4 |
| NO: 286 | GGACUUGGGU | NO: 486 | CACUGUCGA | | |
| SEQ ID | AAGACGCUGGAUC | SEQ ID | AAGAGCCGGAUC | 405-426 | NM_000185.4 |
| NO: 287 | CGGCUCUUGC | NO: 487 | CAGCGUCUU | | |
| SEQ ID | AGUUCUGUUUGUC | SEQ ID | AAAAGCAUGAC | 1194-1215 | NM_000185.4 |
| NO: 288 | AUGCUUUUUU | NO: 488 | AAACAGAACU | | |
| SEQ ID | CUUGGGUGGACAG | SEQ ID | UCAUGCCGCUGU | 1426-1447 | NM_000185.4 |
| NO: 289 | CGGCAUGAAC | NO: 489 | CCACCCAAG | | |
| SEQ ID | UCGAGUUCUGUUU | SEQ ID | AGCAUGACAAAC | 1197-1218 | NM_000185.4 |
| NO: 290 | GUCAUGCUUU | NO: 490 | AGAACUCGA | | |
| SEQ ID | GCCGGAAGUUGUG | SEQ ID | CACACAACCACA | 970-991 | NM_000185.4 |
| NO: 291 | GUUGUGUGUC | NO: 491 | ACUUCCGGC | | |
| SEQ ID | CAGCCGGAAGUUG | SEQ ID | CACAACCACAAC | 972-993 | NM_000185.4 |
| NO: 292 | UGGUUGUGUG | NO: 492 | UUCCGGCUG | | |
| SEQ ID | UAAGACGCUGGAU | SEQ ID | AGAGCCGGAUCC | 406-427 | NM_000185.4 |
| NO: 293 | CCGGCUCUUG | NO: 493 | AGCGUCUUA | | |
| SEQ ID | UCAGCCGGAAGUU | SEQ ID | ACAACCACAACU | 973-994 | NM_000185.4 |
| NO: 294 | GUGGUUGUGU | NO: 494 | UCCGGCUGA | | |
| SEQ ID | UUGGUGCUUGAAC | SEQ ID | AUCGACCUGUUC | 1347-1368 | NM_000185.4 |
| NO: 295 | AGGUCGAUGG | NO: 495 | AAGCACCAA | | |
| SEQ ID | UUUUGCCAUCUCU | SEQ ID | GGUGGUGGAGA | 1175-1196 | NM_000185.4 |
| NO: 296 | CCACCACCCG | NO: 496 | GAUGGCAAAA | | |
| SEQ ID | GACAGUGAAGCGG | SEQ ID | ACCCAAGUCCGC | 1440-1461 | NM_000185.4 |
| NO: 297 | ACUUGGGUGG | NO: 497 | UUCACUGUC | | |
| SEQ ID | UCUCGAGUUCUGU | SEQ ID | CAUGACAAACAG | 1199-1220 | NM_000185.4 |
| NO: 298 | UUGUCAUGCU | NO: 498 | AACUCGAGA | | |
| SEQ ID | UGUUAAGACGCUG | SEQ ID | GCCGGAUCCAGC | 409-430 | NM_000185.4 |
| NO: 299 | GAUCCGGCUC | NO: 499 | GUCUUAACA | | |
| SEQ ID | GACGCUGGAUCCG | SEQ ID | GCAAGAGCCGGA | 403-424 | NM_000185.4 |
| NO: 300 | GCUCUUGCCA | NO: 500 | UCCAGCGUC | | |
| SEQ ID | ACGCUGGAUCCGG | SEQ ID | GGCAAGAGCCGG | 402-423 | NM_000185.4 |
| NO: 301 | CUCUUGCCAU | NO: 501 | AUCCAGCGU | | |
| SEQ ID | UGGACAGCGGCAU | SEQ ID | UGGGGUUCAUG | 1420-1441 | NM_000185.4 |
| NO: 302 | GAACCCCACC | NO: 502 | CCGCUGUCCA | | |
| SEQ ID | AAAUCCUUGUGGA | SEQ ID | UGCCUUCAUCCA | 991-1012 | NM_016186.3 |
| NO: 303 | UGAAGGCAAA | NO: 503 | CAAGGAUUU | | |
| SEQ ID | GCCUCAUGGAGAU | SEQ ID | UGCGAAAGAUC | 756-777 | NM_016186.3 |
| NO: 304 | CUUUCGCAGC | NO: 504 | UCCAUGAGGC | | |
| SEQ ID | UUCUCCAUGAGGA | SEQ ID | GCUGGUGGUCCU | 1390-1411 | NM_016186.3 |
| NO: 305 | CCACCAGCAU | NO: 505 | CAUGGAGAA | | |
| SEQ ID | UGCCUCAUGGAGA | SEQ ID | GCGAAAGAUCUC | 757-778 | NM_016186.3 |
| NO: 306 | UCUUUCGCAG | NO: 506 | CAUGAGGCA | | |
| SEQ ID | AGCAGCUCAUGCA | SEQ ID | GUAUGAGAUGC | 1531-1552 | NM_016186.3 |
| NO: 307 | UCUCAUACUU | NO: 507 | AUGAGCUGCU | | |
| SEQ ID | UCAGGAUUAAUCU | SEQ ID | GUUUGAUGAGA | 1156-1177 | NM_016186.3 |
| NO: 308 | CAUCAAACAG | NO: 508 | UUAAUCCUGA | | |
| SEQ ID | UUGGUUUCAGGA | SEQ ID | UGAGAUUAAUC | 1162-1183 | NM_016186.3 |
| NO: 309 | UUAAUCUCAUC | NO: 509 | CUGAAACCAA | | |
| SEQ ID | GUUUCAGGAUUA | SEQ ID | UGAUGAGAUUA | 1159-1180 | NM_016186.3 |
| NO: 310 | AUCUCAUCAAA | NO: 510 | AUCCUGAAAC | | |
| SEQ ID | UGGUUUCAGGAU | SEQ ID | AUGAGAUUAAU | 1161-1182 | NM_016186.3 |
| NO: 311 | UAAUCUCAUCA | NO: 511 | CCUGAAACCA | | |
| SEQ ID | GGUUUCAGGAUU | SEQ ID | GAUGAGAUUAA | 1160-1181 | NM_016186.3 |
| NO: 312 | AAUCUCAUCAA | NO: 512 | UCCUGAAACC | | |
| SEQ ID | AGGAUUAAUCUCA | SEQ ID | CUGUUUGAUGA | 1154-1175 | NM_016186.3 |
| NO: 313 | UCAAACAGUU | NO: 513 | GAUUAAUCCU | | |
| SEQ ID | UCCUUGUGGAUGA | SEQ ID | UUUUGCCUUCAU | 988-1009 | NM_016186.3 |
| NO: 314 | AGGCAAAACU | NO: 514 | CCACAAGGA | | |
| SEQ ID | GUGCCUCAUGGAG | SEQ ID | CGAAAGAUCUCC | 758-779 | NM_016186.3 |
| NO: 315 | AUCUUUCGCA | NO: 515 | AUGAGGCAC | | |
| SEQ ID | UCUCCAUGAGGAC | SEQ ID | UGCUGGUGGUCC | 1389-1410 | NM_016186.3 |
| NO: 316 | CACCAGCAUG | NO: 516 | UCAUGGAGA | | |
| SEQ ID | CAAAAUCCUUGUG | SEQ ID | CCUUCAUCCACA | 993-1014 | NM_016186.3 |
| NO: 317 | GAUGAAGGCA | NO: 517 | AGGAUUUUG | | |
| SEQ ID | CGUGCCUCAUGGA | SEQ ID | GAAAGAUCUCCA | 759-780 | NM_016186.3 |
| NO: 318 | GAUCUUUCGC | NO: 518 | UGAGGCACG | | |
| SEQ ID | AUCAAAAUCCUUG | SEQ ID | UUCAUCCACAAG | 995-1016 | NM_016186.3 |
| NO: 319 | UGGAUGAAGG | NO: 519 | GAUUUUGAU | | |
| SEQ ID | UUCAGGAUUAAUC | SEQ ID | UUUGAUGAGAU | 1157-1178 | NM_016186.3 |
| NO: 320 | UCAUCAAACA | NO: 520 | UAAUCCUGAA | | |
| SEQ ID | AAUCCUUGUGGAU | SEQ ID | UUGCCUUCAUCC | 990-1011 | NM_016186.3 |
| NO: 321 | GAAGGCAAAA | NO: 521 | ACAAGGAUU | | |
| SEQ ID | CCAUCGUGCCUCA | SEQ ID | GAUCUCCAUGAG | 763-784 | NM_016186.3 |
| NO: 322 | UGGAGAUCUU | NO: 522 | GCACGAUGG | | |
| SEQ ID | UCCAUGAGGACCA | SEQ ID | CAUGCUGGUGG | 1387-1408 | NM_016186.3 |
| NO: 323 | CCAGCAUGGU | NO: 523 | UCCUCAUGGA | | |
| SEQ ID | AUCCUUGUGGAUG | SEQ ID | UUUGCCUUCAUC | 989-1010 | NM_016186.3 |
| NO: 324 | AAGGCAAAAC | NO: 524 | CACAAGGAU | | |
| SEQ ID | AAAAUCCUUGUGG | SEQ ID | GCCUUCAUCCAC | 992-1013 | NM_016186.3 |
| NO: 325 | AUGAAGGCAA | NO: 525 | AAGGAUUUU | | |
| SEQ ID | CAUGGUGGCAUUU | SEQ ID | UACCAAGGAAA | 1370-1391 | NM_016186.3 |
| NO: 326 | CCUUGGUAGG | NO: 526 | UGCCACCAUG | | |
| SEQ ID | UUUCAGGAUUAA | SEQ ID | UUGAUGAGAUU | 1158-1179 | NM_016186.3 |
| NO: 327 | UCUCAUCAAAC | NO: 527 | AAUCCUGAAA | | |
| SEQ ID | UCAAAAUCCUUGU | SEQ ID | CUUCAUCCACAA | 994-1015 | NM_016186.3 |
| NO: 328 | GGAUGAAGGC | NO: 528 | GGAUUUUGA | | |
| SEQ ID | UCGUGCCUCAUGG | SEQ ID | AAAGAUCUCCAU | 760-781 | NM_016186.3 |
| NO: 329 | AGAUCUUUCG | NO: 529 | GAGGCACGA | | |
| SEQ ID | CAGGAUUAAUCUC | SEQ ID | UGUUUGAUGAG | 1155-1176 | NM_016186.3 |
| NO: 330 | AUCAAACAGU | NO: 530 | AUUAAUCCUG | | |
| SEQ ID | CUCCAUGAGGACC | SEQ ID | AUGCUGGUGGU | 1388-1409 | NM_016186.3 |
| NO: 331 | ACCAGCAUGG | NO: 531 | CCUCAUGGAG | | |
| SEQ ID | CCUUGUGGAUGAA | SEQ ID | GUUUUGCCUUCA | 987-1008 | NM_016186.3 |
| NO: 332 | GGCAAAACUC | NO: 532 | UCCACAAGG | | |
| SEQ ID | UGAGGACCACCAG | SEQ ID | CCACCAUGCUGG | 1383-1404 | NM_016186.3 |
| NO: 333 | CAUGGUGGCA | NO: 533 | UGGUCCUCA | | |
| SEQ ID | AUGAGGACCACCA | SEQ ID | CACCAUGCUGGU | 1384-1405 | NM_016186.3 |
| NO: 334 | GCAUGGUGGC | NO: 534 | GGUCCUCAU | | |
| SEQ ID | AUCGUGCCUCAUG | SEQ ID | AAGAUCUCCAUG | 761-782 | NM_016186.3 |
| NO: 335 | GAGAUCUUUC | NO: 535 | AGGCACGAU | | |
| SEQ ID | CUUGUGGAUGAA | SEQ ID | AGUUUUGCCUUC | 986-1007 | NM_016186.3 |
| NO: 336 | GGCAAAACUCC | NO: 536 | AUCCACAAG | | |
| SEQ ID | CAUGAGGACCACC | SEQ ID | ACCAUGCUGGUG | 1385-1406 | NM_016186.3 |
| NO: 337 | AGCAUGGUGG | NO: 537 | GUCCUCAUG | | |
| SEQ ID | CAUCGUGCCUCAU | SEQ ID | AGAUCUCCAUGA | 762-783 | NM_016186.3 |
| NO: 338 | GGAGAUCUUU | NO: 538 | GGCACGAUG | | |
| SEQ ID | CCAUGAGGACCAC | SEQ ID | CCAUGCUGGUGG | 1386-1407 | NM_016186.3 |
| NO: 339 | CAGCAUGGUG | NO: 539 | UCCUCAUGG | | |
| SEQ ID | UUCUUGUCAAAGG | SEQ ID | UGCCUCCACCUU | 1321-1342 | NM_016186.3 |
| NO: 340 | UGGAGGCAAA | NO: 540 | UGACAAGAA | | |
| SEQ ID | AAUUCUUGUCAAA | SEQ ID | CCUCCACCUUUG | 1323-1344 | NM_016186.3 |
| NO: 341 | GGUGGAGGCA | NO: 541 | ACAAGAAUU | | |
| SEQ ID | UACAUCAUGGGCA | SEQ ID | CAUUAAGGUGCC | 1285-1306 | NM_016186.3 |
| NO: 342 | CCUUAAUGGU | NO: 542 | CAUGAUGUA | | |
| SEQ ID | GGCAUUUCCUUGG | SEQ ID | CUGCCCUACCAA | 1364-1385 | NM_016186.3 |
| NO: 343 | UAGGGCAGUU | NO: 543 | GGAAAUGCC | | |
| SEQ ID | UUUCCUUGGUAGG | SEQ ID | CAAACUGCCCUA | 1360-1381 | NM_016186.3 |
| NO: 344 | GCAGUUUGAG | NO: 544 | CCAAGGAAA | | |
| SEQ ID | UUCCUUGGUAGGG | SEQ ID | UCAAACUGCCCU | 1359-1380 | NM_016186.3 |
| NO: 345 | CAGUUUGAGG | NO: 545 | ACCAAGGAA | | |
| SEQ ID | AAAUUCUUGUCAA | SEQ ID | CUCCACCUUUGA | 1324-1345 | NM_016186.3 |
| NO: 346 | AGGUGGAGGC | NO: 546 | CAAGAAUUU | | |
| SEQ ID | UUGUCCAGGUGGA | SEQ ID | CGACACUUUCCA | 1255-1276 | NM_016186.3 |
| NO: 347 | AAGUGUCGAC | NO: 547 | CCUGGACAA | | |
| SEQ ID | GUACUUGUCCAGG | SEQ ID | ACUUUCCACCUG | 1259-1280 | NM_016186.3 |
| NO: 348 | UGGAAAGUGU | NO: 548 | GACAAGUAC | | |
| SEQ ID | AUUUCCUUGGUAG | SEQ ID | AAACUGCCCUAC | 1361-1382 | NM_016186.3 |
| NO: 349 | GGCAGUUUGA | NO: 549 | CAAGGAAAU | | |
| SEQ ID | GCAUUUCCUUGGU | SEQ ID | ACUGCCCUACCA | 1363-1384 | NM_016186.3 |
| NO: 350 | AGGGCAGUUU | NO: 550 | AGGAAAUGC | | |
| SEQ ID | UACUUGUCCAGGU | SEQ ID | CACUUUCCACCU | 1258-1279 | NM_016186.3 |
| NO: 351 | GGAAAGUGUC | NO: 551 | GGACAAGUA | | |
| SEQ ID | ACAUCAUGGGCAC | SEQ ID | CCAUUAAGGUGC | 1284-1305 | NM_016186.3 |
| NO: 352 | CUUAAUGGUC | NO: 552 | CCAUGAUGU | | |
| SEQ ID | UCUUGUCAAAGGU | SEQ ID | UUGCCUCCACCU | 1320-1341 | NM_016186.3 |
| NO: 353 | GGAGGCAAAC | NO: 553 | UUGACAAGA | | |
| SEQ ID | UUGUACUUGUCCA | SEQ ID | UUUCCACCUGGA | 1261-1282 | NM_016186.3 |
| NO: 354 | GGUGGAAAGU | NO: 554 | CAAGUACAA | | |
| SEQ ID | CUUGGUAGGGCAG | SEQ ID | UCCUCAAACUGC | 1356-1377 | NM_016186.3 |
| NO: 355 | UUUGAGGACA | NO: 555 | CCUACCAAG | | |
| SEQ ID | CAUUUCCUUGGUA | SEQ ID | AACUGCCCUACC | 1362-1383 | NM_016186.3 |
| NO: 356 | GGGCAGUUUG | NO: 556 | AAGGAAAUG | | |
| SEQ ID | CCUUGGUAGGGCA | SEQ ID | CCUCAAACUGCC | 1357-1378 | NM_016186.3 |
| NO: 357 | GUUUGAGGAC | NO: 557 | CUACCAAGG | | |
| SEQ ID | UUGGUAGGGCAG | SEQ ID | GUCCUCAAACUG | 1355-1376 | NM_016186.3 |
| NO: 358 | UUUGAGGACAU | NO: 558 | CCCUACCAA | | |
| SEQ ID | CUUGUCCAGGUGG | SEQ ID | GACACUUUCCAC | 1256-1277 | NM_016186.3 |
| NO: 359 | AAAGUGUCGA | NO: 559 | CUGGACAAG | | |
| SEQ ID | CUUGUCAAAGGUG | SEQ ID | UUUGCCUCCACC | 1319-1340 | NM_016186.3 |
| NO: 360 | GAGGCAAACU | NO: 560 | UUUGACAAG | | |
| SEQ ID | ACUUGUCCAGGUG | SEQ ID | ACACUUUCCACC | 1257-1278 | NM_016186.3 |
| NO: 361 | GAAAGUGUCG | NO: 561 | UGGACAAGU | | |
| SEQ ID | CUUGUACUUGUCC | SEQ ID | UUCCACCUGGAC | 1262-1283 | NM_016186.3 |
| NO: 362 | AGGUGGAAAG | NO: 562 | AAGUACAAG | | |
| SEQ ID | UGUACUUGUCCAG | SEQ ID | CUUUCCACCUGG | 1260-1281 | NM_016186.3 |
| NO: 363 | GUGGAAAGUG | NO: 563 | ACAAGUACA | | |
| SEQ ID | UCCUUGGUAGGGC | SEQ ID | CUCAAACUGCCC | 1358-1379 | NM_016186.3 |
| NO: 364 | AGUUUGAGGA | NO: 564 | UACCAAGGA | | |
| SEQ ID | UUCCCUUUGAACA | SEQ ID | UUACAUCUUGU | 1198-1219 | NM_016186.3 |
| NO: 365 | AGAUGUAAUC | NO: 565 | UCAAAGGGAA | | |
| SEQ ID | AGGACCACCAGCA | SEQ ID | UGCCACCAUGCU | 1381-1402 | NM_016186.3 |
| NO: 366 | UGGUGGCAUU | NO: 566 | GGUGGUCCU | | |
| SEQ ID | UAUCAAAAUACCU | SEQ ID | UAUCCAAGAGG | 1038-1059 | NM_016186.3 |
| NO: 367 | CUUGGAUAAA | NO: 567 | UAUUUUGAUA | | |
| SEQ ID | ACCACCAGCAUGG | SEQ ID | AAAUGCCACCAU | 1378-1399 | NM_016186.3 |
| NO: 368 | UGGCAUUUCC | NO: 568 | GCUGGUGGU | | |
| SEQ ID | UCCCUUUGAACAA | SEQ ID | AUUACAUCUUG | 1197-1218 | NM_016186.3 |
| NO: 369 | GAUGUAAUCC | NO: 569 | UUCAAAGGGA | | |
| SEQ ID | UUGCCAUCGUGCC | SEQ ID | CUCCAUGAGGCA | 766-787 | NM_016186.3 |
| NO: 370 | UCAUGGAGAU | NO: 570 | CGAUGGCAA | | |
| SEQ ID | UUUGAUGACAGG | SEQ ID | UCCAUGCCUCCU | 1721-1742 | NM_016186.3 |
| NO: 371 | AGGCAUGGAAU | NO: 571 | GUCAUCAAA | | |
| SEQ ID | UGAUGACAGGAG | SEQ ID | AUUCCAUGCCUC | 1719-1740 | NM_016186.3 |
| NO: 372 | GCAUGGAAUAA | NO: 572 | CUGUCAUCA | | |
| SEQ ID | CAGCAUGGUGGCA | SEQ ID | CAAGGAAAUGCC | 1373-1394 | NM_016186.3 |
| NO: 373 | UUUCCUUGGU | NO: 573 | ACCAUGCUG | | |
| SEQ ID | UUUUCUCCAUGAG | SEQ ID | UGGUGGUCCUCA | 1392-1413 | NM_016186.3 |
| NO: 374 | GACCACCAGC | NO: 574 | UGGAGAAAA | | |
| SEQ ID | CCAUUUCCCUUUG | SEQ ID | AUCUUGUUCAA | 1202-1223 | NM_016186.3 |
| NO: 375 | AACAAGAUGU | NO: 575 | AGGGAAAUGG | | |
| SEQ ID | CCAGCAUGGUGGC | SEQ ID | AAGGAAAUGCC | 1374-1395 | NM_016186.3 |
| NO: 376 | AUUUCCUUGG | NO: 576 | ACCAUGCUGG | | |
| SEQ ID | AUUCUUGUCAAAG | SEQ ID | GCCUCCACCUUU | 1322-1343 | NM_016186.3 |
| NO: 377 | GUGGAGGCAA | NO: 577 | GACAAGAAU | | |
| SEQ ID | UUGUGGAUGAAG | SEQ ID | GAGUUUUGCCU | 985-1006 | NM_016186.3 |
| NO: 378 | GCAAAACUCCC | NO: 578 | UCAUCCACAA | | |
| SEQ ID | CUCAUGGAGAUCU | SEQ ID | GCUGCGAAAGA | 754-775 | NM_016186.3 |
| NO: 379 | UUCGCAGCAG | NO: 579 | UCUCCAUGAG | | |
| SEQ ID | UGUCAAAGGUGG | SEQ ID | AGUUUGCCUCCA | 1317-1338 | NM_016186.3 |
| NO: 380 | AGGCAAACUUG | NO: 580 | CCUUUGACA | | |
| SEQ ID | UUGUCAAAGGUG | SEQ ID | GUUUGCCUCCAC | 1318-1339 | NM_016186.3 |
| NO: 381 | GAGGCAAACUU | NO: 581 | CUUUGACAA | | |
| SEQ ID | CAUUUCCCUUUGA | SEQ ID | CAUCUUGUUCAA | 1201-1222 | NM_016186.3 |
| NO: 382 | ACAAGAUGUA | NO: 582 | AGGGAAAUG | | |
| SEQ ID | UGUGGAUGAAGG | SEQ ID | GGAGUUUUGCC | 984-1005 | NM_016186.3 |
| NO: 383 | CAAAACUCCCC | NO: 583 | UUCAUCCACA | | |
| SEQ ID | CCUCAUGGAGAUC | SEQ ID | CUGCGAAAGAUC | 755-776 | NM_016186.3 |
| NO: 384 | UUUCGCAGCA | NO: 584 | UCCAUGAGG | | |
| SEQ ID | CUUUGAUGACAGG | SEQ ID | CCAUGCCUCCUG | 1722-1743 | NM_016186.3 |
| NO: 385 | AGGCAUGGAA | NO: 585 | UCAUCAAAG | | |
| SEQ ID | UCACCACCCUGCC | SEQ ID | UGUUUCUGGGC | 1794-1815 | NM_016186.3 |
| NO: 386 | CAGAAACAGA | NO: 586 | AGGGUGGUGA | | |
| SEQ ID | UGGAGAUCUUUCG | SEQ ID | GCCUGCUGCGAA | 750-771 | NM_016186.3 |
| NO: 387 | CAGCAGGCUG | NO: 587 | AGAUCUCCA | | |
| SEQ ID | GAUUAAUCUCAUC | SEQ ID | AACUGUUUGAU | 1152-1173 | NM_016186.3 |
| NO: 388 | AAACAGUUUG | NO: 588 | GAGAUUAAUC | | |
| SEQ ID | CACUUUGAUGACA | SEQ ID | AUGCCUCCUGUC | 1724-1745 | NM_016186.3 |
| NO: 389 | GGAGGCAUGG | NO: 589 | AUCAAAGUG | | |
| SEQ ID | GCAGCUCAUGCAU | SEQ ID | AGUAUGAGAUG | 1530-1551 | NM_016186.3 |
| NO: 390 | CUCAUACUUC | NO: 590 | CAUGAGCUGC | | |
| SEQ ID | CAGCUCAUGCAUC | SEQ ID | AAGUAUGAGAU | 1529-1550 | NM_016186.3 |
| NO: 391 | UCAUACUUCU | NO: 591 | GCAUGAGCUG | | |
| SEQ ID | UGGUAGGGCAGU | SEQ ID | UGUCCUCAAACU | 1354-1375 | NM_016186.3 |
| NO: 392 | UUGAGGACAUG | NO: 592 | GCCCUACCA | | |
| SEQ ID | GGAUUAAUCUCAU | SEQ ID | ACUGUUUGAUG | 1153-1174 | NM_016186.3 |
| NO: 393 | CAAACAGUUU | NO: 593 | AGAUUAAUCC | | |
| SEQ ID | AUCAUGGGCACCU | SEQ ID | GACCAUUAAGG | 1282-1303 | NM_016186.3 |
| NO: 394 | UAAUGGUCUU | NO: 594 | UGCCCAUGAU | | |
| SEQ ID | CAUCAUGGGCACC | SEQ ID | ACCAUUAAGGU | 1283-1304 | NM_016186.3 |
| NO: 395 | UUAAUGGUCU | NO: 595 | GCCCAUGAUG | | |
| SEQ ID | UUCACCACCCUGC | SEQ ID | GUUUCUGGGCA | 1795-1816 | NM_016186.3 |
| NO: 396 | CCAGAAACAG | NO: 596 | GGGUGGUGAA | | |
| SEQ ID | CCACCCUGCCCAG | SEQ ID | UUCUGUUUCUG | 1791-1812 | NM_016186.3 |
| NO: 397 | AAACAGAAGC | NO: 597 | GGCAGGGUGG | | |
| SEQ ID | GCUUGAACUUCGG | SEQ ID | UUUUCUUUCCGA | 1500-1521 | NM_016186.3 |
| NO: 398 | AAAGAAAACU | NO: 598 | AGUUCAAGC | | |
| SEQ ID | AGCUUGAACUUCG | SEQ ID | UUUCUUUCCGAA | 1501-1522 | NM_016186.3 |
| NO: 399 | GAAAGAAAAC | NO: 599 | GUUCAAGCU | | |
| SEQ ID | ACCACCCUGCCCA | SEQ ID | UCUGUUUCUGG | 1792-1813 | NM_016186.3 |
| NO: 400 | GAAACAGAAG | NO: 600 | GCAGGGUGGU | | |
| SEQ ID | GGUAGGGCAGUU | SEQ ID | AUGUCCUCAAAC | 1353-1374 | NM_016186.3 |
| NO: 401 | UGAGGACAUGA | NO: 601 | UGCCCUACC | | |
| SEQ ID | UGUCCAGGUGGAA | SEQ ID | UCGACACUUUCC | 1254-1275 | NM_016186.3 |
| NO: 402 | AGUGUCGACU | NO: 602 | ACCUGGACA | | |

Table 24 provides the modified first (antisense) sequences, together with the corresponding unmodified first (antisense) sequences for siRNA oligonucleosides (targeting HCII and ZPI) according to the present invention as follows.

TABLE 24

| | | | Underlying Base Sequence | |
|---|---|---|---|---|
| | Modified First (Antisense) | SEQ ID | 5′ → 3′ | SEQ ID |
| Antisense | Strand | NO (AS - | (Shown as an Unmodified | NO (AS - |
| strand ID | 5′ → 3′ | mod) | Nucleoside Sequence) | unmod) |
| ETXS232 | UmsUfsUmCmCmAfCmUf | SEQ ID | UUUCCACUGGGAAUU | SEQ ID |
| | GfGmGmAmAmUfUmUfA | NO: 603 | UAUUCACC | NO: 203 |
| | mUmUmCmAmsCmsCm | | | |
| ETXS234 | GmsAfsUmCmCmUfUmUf | SEQ ID | GAUCCUUUGAAGUAG | SEQ ID |
| | GfAmAmGmUmAfGmAfU | NO: 604 | AUGCAGUU | NO: 204 |
| | mGmCmAmGmsUmsUm | | | |
| ETXS236 | AmsUfsCmCmUmUfUmGf | SEQ ID | AUCCUUUGAAGUAGA | SEQ ID |
| | AfAmGmUmAmGfAmUfG | NO: 605 | UGCAGUUG | NO: 205 |
| | mCmAmGmUmsUmsGm | | | |
| ETXS238 | AmsCfsUmUmGmUfUmCf | SEQ ID | ACUUGUUCAUGGGUC | SEQ ID |
| | AfUmGmGmGmUfCmUfC | NO: 606 | UCUCCCUU | NO: 206 |
| | mUmCmCmCmsUmsUm | | | |
| ETXS240 | UmsCfsCmAmCmUfGmGf | SEQ ID | UCCACUGGGAAUUUA | SEQ ID |
| | GfAmAmUmUmUfAmUfU | NO: 607 | UUCACCCA | NO: 207 |
| | mCmAmCmCmsCmsAm | | | |
| ETXS242 | UmsGfsUmAmCmCfCmAf | SEQ ID | UGUACCCAAAAUUCC | SEQ ID |
| | AfAmAmUmUmCfCmUfC | NO: 608 | UCCUGAAG | NO: 208 |
| | mCmUmGmAmsAmsGm | | | |
| ETXS244 | UmsUfsCmCmCmAfUmGf | SEQ ID | UUCCCAUGAAGAGCA | SEQ ID |
| | AfAmGmAmGmCfAmGfG | NO: 609 | GGCAGCUG | NO: 209 |
| | mCmAmGmCmsUmsGm | | | |
| ETXS246 | UmsCfsUmUmUmUfAmUf | SEQ ID | UCUUUUAUGAGGCCC | SEQ ID |
| | GfAmGmGmCmCfCmUfU | NO: 610 | UUGGUGAG | NO: 210 |
| | mGmGmUmGmsAmsGm | | | |
| ETXS248 | AmsAfsCmUmGmCfUmUf | SEQ ID | AACUGCUUCUGGAUA | SEQ ID |
| | CfUmGmGmAmUfAmUfA | NO: 611 | UAAAGGUC | NO: 211 |
| | mAmAmGmGmsUmsCm | | | |
| ETXS250 | CmsCfsUmUmUmGfAmAf | SEQ ID | CCUUUGAAGUAGAUG | SEQ ID |
| | GfUmAmGmAmUfGmCfA | NO: 612 | CAGUUGAG | NO: 212 |
| | mGmUmUmGmsAmsGm | | | |
| ETXS252 | UmsUfsAmAmGmAfCmGf | SEQ ID | UUAAGACGCUGGAUC | SEQ ID |
| | CfUmGmGmAmUfCmCfG | NO: 613 | CGGCUCUU | NO: 213 |
| | mGmCmUmCmsUmsUm | | | |
| ETXS254 | GmsAfsGmGmAmUfGmUf | SEQ ID | GAGGAUGUUAAGACG | SEQ ID |
| | UfAmAmGmAmCfGmCfU | NO: 614 | CUGGAUCC | NO: 214 |
| | mGmGmAmUmsCmsCm | | | |
| ETXS256 | UmsCfsUmGmUmUfUmGf | SEQ ID | UCUGUUUGUCAUGCU | SEQ ID |
| | UfCmAmUmGmCfUmUfU | NO: 615 | | |
| | mUmUmUmGmsCmsCm | | UUUUUGCC | NO: 215 |
| ETXS258 | GmsUfsGmUmAmCfCmCf | SEQ ID | GUGUACCCAAAAUUC | SEQ ID |
| | AfAmAmAmUmUfCmCfU | NO: 616 | CUCCUGAA | NO: 216 |
| | mCmCmUmGmsAmsAm | | | |
| ETXS260 | UmsUfsCmAmGmCfCmGf | SEQ ID | UUCAGCCGGAAGUUG | SEQ ID |
| | GfAmAmGmUmUfGmUfG | NO: 617 | UGGUUGUG | NO: 217 |
| | mGmUmUmGmsUmsGm | | | |
| ETXS262 | UmsGfsUmUmUmGfUmCf | SEQ ID | UGUUUGUCAUGCUUU | SEQ ID |
| | AfUmGmCmUmUfUmUfU | NO: 618 | UUUGCCAU | NO: 218 |
| | mUmGmCmCmsAmsUm | | | |
| ETXS264 | AmsUfsUmUmCmCfAmCf | SEQ ID | AUUUCCACUGGGAAU | SEQ ID |
| | UfGmGmGmAmAfUmUfU | NO: 619 | UUAUUCAC | NO: 219 |
| | mAmUmUmCmsAmsCm | | | |
| ETXS266 | AmsCfsAmAmUmUfAmGf | SEQ ID | ACAAUUAGCAUGCUG | SEQ ID |
| | CfAmUmGmCmUfGmAfU | NO: 620 | | NO: 220 |
| | mGmCmCmCmsCmsCm | | AUGCCCCC | |
| ETXS268 | GmsUfsUmCmUmGfUmUf | SEQ ID | GUUCUGUUUGUCAUG | SEQ ID |
| | UfGmUmCmAmUfGmCfU | NO: 621 | CUUUUUUG | NO: 221 |
| | mUmUmUmUmsUmsGm | | | |
| ETXS270 | GmsGfsCmCmUmCfAmGf | SEQ ID | GGCCUCAGCAAAGUA | SEQ ID |
| | CfAmAmAmGmUfAmAfU | NO: 622 | AUACUCUC | NO: 222 |
| | mAmCmUmCmsUmsCm | | | |
| ETXS272 | CmsUfsCmUmCmCfCmUf | SEQ ID | CUCUCCCUUCAGACC | SEQ ID |
| | UfCmAmGmAmCfCmUfA | NO: 623 | UAAGGAAA | NO: 223 |
| | mAmGmGmAmsAmsAm | | | |
| ETXS274 | UmsGfsGmUmGmCfUmUf | SEQ ID | UGGUGCUUGAACAGG | SEQ ID |
| | GfAmAmCmAmGfGmUfC | NO: 624 | UCGAUGGC | NO: 224 |
| | mGmAmUmGmsGmsCm | | | |
| ETXS276 | CmsUfsUmUmUmAfUmGf | SEQ ID | CUUUUAUGAGGCCCU | SEQ ID |
| | AfGmGmCmCmCfUmUfG | NO: 625 | UGGUGAGC | NO: 225 |
| | mGmUmGmAmsGmsCm | | | |
| ETXS278 | CmsAfsUmUmUmCfCmAf | SEQ ID | CAUUUCCACUGGGAA | SEQ ID |
| | CfUmGmGmGmAfAmUfU | NO: 626 | UUUAUUCA | NO: 226 |
| | mUmAmUmUmsCmsAm | | | |
| ETXS280 | CmsUfsGmUmUmUfGmUf | SEQ ID | CUGUUUGUCAUGCUU | SEQ ID |
| | CfAmUmGmCmUfUmUfU | NO: 627 | UUUUGCCA | NO: 227 |
| | mUmUmGmCmsCmsAm | | | |
| ETXS282 | CmsUfsCmUmCmUfCmUf | SEQ ID | CUCUCUCUCAUUCAG | SEQ ID |
| | CfAmUmUmCmAfGmCfC | NO: 628 | CCGGAAGU | NO: 228 |
| | mGmGmAmAmsGmsUm | | | |
| ETXS284 | CmsUfsCmGmAmGfUmUf | SEQ ID | CUCGAGUUCUGUUUG | SEQ ID |
| | CfUmGmUmUmUfGmUfC | NO: 629 | UCAUGCUU | NO: 229 |
| | mAmUmGmCmsUmsUm | | | |
| ETXS286 | CmsAfsCmUmCmUfUmCf | SEQ ID | CACUCUUCCCAUGAA | SEQ ID |
| | CfCmAmUmGmAfAmGfA | NO: 630 | GAGCAGGC | NO: 230 |
| | mGmCmAmGmsGmsCm | | | |
| ETXS288 | GmsCfsCmUmCmAfGmCf | SEQ ID | GCCUCAGCAAAGUAA | SEQ ID |
| | AfAmAmGmUmAfAmUfA | NO: 631 | UACUCUCU | NO: 231 |
| | mCmUmCmUmsCmsUm | | | |
| ETXS290 | UmsAfsCmCmCmAfAmAf | SEQ ID | UACCCAAAAUUCCUC | SEQ ID |
| | AfUmUmCmCmUfCmCfU | NO: 632 | CUGAAGAG | NO: 232 |
| | mGmAmAmGmsAmsGm | | | |
| ETXS292 | CmsGfsAmGmUmUfCmUf | SEQ ID | CGAGUUCUGUUUGUC | SEQ ID |
| | GfUmUmUmGmUfCmAfU | NO: 633 | AUGCUUUU | NO: 233 |
| | mGmCmUmUmsUmsUm | | | |
| ETXS294 | UmsUfsCmCmAmCfUmGf | SEQ ID | UUCCACUGGGAAUUU | SEQ ID |
| | GfGmAmAmUmUfUmAfU | NO: 634 | AUUCACCC | NO: 234 |
| | mUmCmAmCmsCmsCm | | | |
| ETXS296 | GmsGfsAmUmGmUfUmAf | SEQ ID | GGAUGUUAAGACGCU | SEQ ID |
| | AfGmAmCmGmCfUmGfG | NO: 635 | GGAUCCGG | NO: 235 |
| | mAmUmCmCmsGmsGm | | | |
| ETXS298 | AmsCfsUmUmCmUfCmGf | SEQ ID | ACUUCUCGAGUUCUG | SEQ ID |
| | AfGmUmUmCmUfGmUfU | NO: 636 | UUUGUCAU | NO: 236 |
| | mUmGmUmCmsAmsUm | | | |
| ETXS300 | GmsGfsCmGmAmUfCmCf | SEQ ID | GGCGAUCCUUUGGUC | SEQ ID |
| | UfUmUmGmGmUfCmUfG | NO: 637 | UGAGAUGC | NO: 237 |
| | mAmGmAmUmsGmsCm | | | |
| ETXS302 | CmsUfsCmUmCmAfUmUf | SEQ ID | CUCUCAUUCAGCCGG | SEQ ID |
| | CfAmGmCmCmGfGmAfA | NO: 638 | AAGUUGUG | NO: 238 |
| | mGmUmUmGmsUmsGm | | | |
| ETXS304 | GmsUfsAmCmCmCfAmAf | SEQ ID | GUACCCAAAAUUCCU | SEQ ID |
| | AfAmUmUmCmCfUmCfC | NO: 639 | CCUGAAGA | NO: 239 |
| | mUmGmAmAmsGmsAm | | | |
| ETXS306 | CmsCfsUmCmAmGfCmAf | SEQ ID | CCUCAGCAAAGUAAU | SEQ ID |
| | AfAmGmUmAmAfUmAfC | NO: 640 | ACUCUCUU | NO: 240 |
| | mUmCmUmCmsUmsUm | | | |
| ETXS308 | UmsCfsAmUmUmCfAmGf | SEQ ID | UCAUUCAGCCGGAAG | SEQ ID |
| | CfCmGmGmAmAfGmUfU | NO: 641 | UUGUGGUU | NO: 241 |
| | mGmUmGmGmsUmsUm | | | |
| ETXS310 | GmsAfsUmGmUmUfAmAf | SEQ ID | GAUGUUAAGACGCUG | SEQ ID |
| | GfAmCmGmCmUfGmGfA | NO: 642 | GAUCCGGC | NO: 242 |
| | mUmCmCmGmsGmsCm | | | |
| ETXS312 | UmsUfsGmAmAmGfUmAf | SEQ ID | UUGAAGUAGAUGCAG | SEQ ID |
| | GfAmUmGmCmAfGmUfU | NO: 643 | UUGAGAAU | NO: 243 |
| | mGmAmGmAmsAmsUm | | | |
| ETXS314 | CmsGfsAmUmCmCfUmUf | SEQ ID | CGAUCCUUUGGUCUG | SEQ ID |
| | UfGmGmUmCmUfGmAfG | NO: 644 | AGAUGCCU | NO: 244 |
| | mAmUmGmCmsCmsUm | | | |
| ETXS316 | GmsAfsGmUmUmCfUmGf | SEQ ID | GAGUUCUGUUUGUCA | SEQ ID |
| | UfUmUmGmUmCfAmUfG | NO: 645 | UGCUUUUU | NO: 245 |
| | mCmUmUmUmsUmsUm | | | |
| ETXS318 | GmsCfsAmGmGmGfUmCf | SEQ ID | GCAGGGUCUAUAUUC | SEQ ID |
| | UfAmUmAmUmUfCmUfC | NO: 646 | UCCAGAGC | NO: 246 |
| | mCmAmGmAmsGmsCm | | | |
| ETXS320 | CmsUfsCmAmGmCfAmAf | SEQ ID | CUCAGCAAAGUAAUA | SEQ ID |
| | AfGmUmAmAmUfAmCfU | NO: 647 | CUCUCUUA | NO: 247 |
| | mCmUmCmUmsUmsAm | | | |
| ETXS322 | AmsCfsCmUmCmUfCmUf | SEQ ID | ACCUCUCUCUCAUUC | SEQ ID |
| | CfUmCmAmUmUfCmAfG | NO: 648 | AGCCGGAA | NO: 248 |
| | mCmCmGmGmsAmsAm | | | |
| ETXS324 | CmsUfsGmGmGmAfAmUf | SEQ ID | CUGGGAAUUUAUUCA | SEQ ID |
| | UfUmAmUmUmCfAmCfC | NO: 649 | CCCAGGAU | NO: 249 |
| | mCmAmGmGmsAmsUm | | | |
| ETXS326 | GmsUfsCmUmCmUfCmCf | SEQ ID | GUCUCUCCCUUCAGA | SEQ ID |
| | CfUmUmCmAmGfAmCfC | NO: 650 | CCUAAGGA | NO: 250 |
| | mUmAmAmGmsGmsAm | | | |
| ETXS328 | UmsCfsUmCmUmCfUmCf | SEQ ID | UCUCUCUCAUUCAGC | SEQ ID |
| | AfUmUmCmAmGfCmCfG | NO: 651 | CGGAAGUU | NO: 251 |
| | mGmAmAmGmsUmsUm | | | |
| ETXS330 | GmsUfsCmAmUmUfUmCf | SEQ ID | GUCAUUUCCACUGGG | SEQ ID |
| | CfAmCmUmGmGfGmAfA | NO: 652 | AAUUUAUU | NO: 252 |
| | mUmUmUmAmsUmsUm | | | |
| ETXS332 | AmsUfsUmCmAmGfCmCf | SEQ ID | AUUCAGCCGGAAGUU | SEQ ID |
| | GfGmAmAmGmUfUmGfU | NO: 653 | GUGGUUGU | NO: 253 |
| | mGmGmUmUmsGmsUm | | | |
| ETXS334 | GmsUfsAmGmCmAfGmGf | SEQ ID | GUAGCAGGGUCUAUA | SEQ ID |
| | GfUmCmUmAmUfAmUfU | NO: 654 | UUCUCCAG | NO: 254 |
| | mCmUmCmCmsAmsGm | | | |
| ETXS336 | GmsGfsUmGmCmUfUmGf | SEQ ID | GGUGCUUGAACAGGU | SEQ ID |
| | AfAmCmAmGmGfUmCfG | NO: 655 | CGAUGGCG | NO: 255 |
| | mAmUmGmGmsCmsGm | | | |
| ETXS338 | UmsGfsAmAmGmUfAmGf | SEQ ID | UGAAGUAGAUGCAGU | SEQ ID |
| | AfUmGmCmAmGfUmUfG | NO: 656 | UGAGAAUC | NO: 256 |
| | mAmGmAmAmsUmsCm | | | |
| ETXS340 | UmsGfsAmGmGmAfUmGf | SEQ ID | UGAGGAUGUUAAGAC | SEQ ID |
| | UfUmAmAmGmAfCmGfC | NO: 657 | GCUGGAUC | NO: 257 |
| | mUmGmGmAmsUmsCm | | | |
| ETXS342 | AmsUfsCmCmUmUfUmGf | SEQ ID | AUCCUUUGGUCUGAG | SEQ ID |
| | GfUmCmUmGmAfGmAfU | NO: 658 | AUGCCUGC | NO: 258 |
| | mGmCmCmUmsGmsCm | | | |
| ETXS344 | CmsGfsGmAmCmUfUmGf | SEQ ID | CGGACUUGGGUGGAC | SEQ ID |
| | GfGmUmGmGmAfCmAfG | NO: 659 | AGCGGCAU | NO: 259 |
| | mCmGmGmCmsAmsUm | | | |
| ETXS346 | UmsUfsCmUmCmGfAmGf | SEQ ID | UUCUCGAGUUCUGUU | SEQ ID |
| | UfUmCmUmGmUfUmUfG | NO: 660 | UGUCAUGC | NO: 260 |
| | mUmCmAmUmsGmsCm | | | |
| ETXS348 | GmsUfsGmGmAmCfAmGf | SEQ ID | GUGGACAGCGGCAUG | SEQ ID |
| | CfGmGmCmAmUfGmAfA | NO: 661 | AACCCCAC | NO: 261 |
| | mCmCmCmCmsAmsCm | | | |
| ETXS350 | AmsGfsGmAmUmGfUmUf | SEQ ID | AGGAUGUUAAGACGC | SEQ ID |
| | AfAmGmAmCmGfCmUfG | NO: 662 | UGGAUCCG | NO: 262 |
| | mGmAmUmCmsCmsGm | | | |
| ETXS352 | CmsGfsAmCmAmGfUmGf | SEQ ID | CGACAGUGAAGCGGA | SEQ ID |
| | AfAmGmCmGmGfAmCfU | NO: 663 | CUUGGGUG | NO: 263 |
| | mUmGmGmGmsUmsGm | | | |
| ETXS354 | CmsUfsCmUmCmUfCmAf | SEQ ID | CUCUCUCAUUCAGCC | SEQ ID |
| | UfUmCmAmGmCfCmGfG | NO: 664 | GGAAGUUG | NO: 264 |
| | mAmAmGmUmsUmsGm | | | |
| ETXS356 | CmsUfsGmGmGmCfCmUf | SEQ ID | CUGGGCCUCAGCAAA | SEQ ID |
| | CfAmGmCmAmAfAmGfU | NO: 665 | GUAAUACU | NO: 265 |
| | mAmAmUmAmsCmsUm | | | |
| ETXS358 | AmsGfsCmAmGmGfGmUf | SEQ ID | AGCAGGGUCUAUAUU | SEQ ID |
| | CfUmAmUmAmUfUmCfU | NO: 666 | CUCCAGAG | NO: 266 |
| | mCmCmAmGmsAmsGm | | | |
| ETXS360 | AmsGfsUmAmGmAfUmGf | SEQ ID | AGUAGAUGCAGUUGA | SEQ ID |
| | CfAmGmUmUmGfAmGfA | NO: 667 | GAAUCAUC | NO: 267 |
| | mAmUmCmAmsUmsCm | | | |
| ETXS362 | GmsGfsAmCmUmUfGmGf | SEQ ID | GGACUUGGGUGGACA | SEQ ID |
| | GfUmGmGmAmCfAmGfC | NO: 668 | GCGGCAUG | NO: 268 |
| | mGmGmCmAmsUmsGm | | | |
| ETXS364 | UmsUfsUmGmAmAfGmUf | SEQ ID | UUUGAAGUAGAUGCA | SEQ ID |
| | AfGmAmUmGmCfAmGfU | NO: 669 | GUUGAGAA | NO: 269 |
| | mUmGmAmGmsAmsAm | | | |
| ETXS366 | CmsAfsUmUmCmAfGmCf | SEQ ID | CAUUCAGCCGGAAGU | SEQ ID |
| | CfGmGmAmAmGfUmUfG | NO: 670 | UGUGGUUG | NO: 270 |
| | mUmGmGmUmsUmsGm | | | |
| ETXS368 | AmsCfsAmGmCmGfGmCf | SEQ ID | ACAGCGGCAUGAACC | SEQ ID |
| | AfUmGmAmAmCfCmCfC | NO: 671 | CCACCGUG | NO: 271 |
| | mAmCmCmGmsUmsGm | | | |
| ETXS370 | CmsCfsUmCmUmCfUmCf | SEQ ID | CCUCUCUCUCAUUCA | SEQ ID |
| | UfCmAmUmUmCfAmGfC | NO: 672 | GCCGGAAG | NO: 272 |
| | mCmGmGmAmsAmsGm | | | |
| ETXS372 | AmsCfsCmCmAmAfAmAf | SEQ ID | ACCCAAAAUUCCUCC | SEQ ID |
| | UfUmCmCmUmCfCmUfG | NO: 673 | UGAAGAGG | NO: 273 |
| | mAmAmGmAmsGmsGm | | | |
| ETXS374 | UmsGfsGmGmCmCfUmCf | SEQ ID | UGGGCCUCAGCAAAG | SEQ ID |
| | AfGmCmAmAmAfGmUfA | NO: 674 | UAAUACUC | NO: 274 |
| | mAmUmAmCmsUmsCm | | | |
| ETXS376 | AmsAfsGmUmAmGfAmUf | SEQ ID | AAGUAGAUGCAGUUG | SEQ ID |
| | GfCmAmGmUmUfGmAfG | NO: 675 | AGAAUCAU | NO: 275 |
| | mAmAmUmCmsAmsUm | | | |
| ETXS378 | GmsCfsGmAmUmCfCmUf | SEQ ID | GCGAUCCUUUGGUCU | SEQ ID |
| | UfUmGmGmUmCfUmGfA | NO: 676 | GAGAUGCC | NO: 276 |
| | mGmAmUmGmsCmsCm | | | |
| ETXS380 | GmsAfsUmCmCmUfUmUf | SEQ ID | GAUCCUUUGGUCUGA | SEQ ID |
| | GfGmUmCmUmGfAmGfA | NO: 677 | GAUGCCUG | NO: 277 |
| | mUmGmCmCmsUmsGm | | | |
| ETXS382 | CmsUfsUmUmGmAfAmGf | SEQ ID | CUUUGAAGUAGAUGC | SEQ ID |
| | UfAmGmAmUmGfCmAfG | NO: 678 | AGUUGAGA | NO: 278 |
| | mUmUmGmAmsGmsAm | | | |
| ETXS384 | GmsUfsCmGmAmCfAmGf | SEQ ID | GUCGACAGUGAAGCG | SEQ ID |
| | UfGmAmAmGmCfGmGfA | NO: 679 | GACUUGGG | NO: 279 |
| | mCmUmUmGmsGmsGm | | | |
| ETXS386 | CmsAfsAmUmUmAfGmCf | SEQ ID | CAAUUAGCAUGCUGA | SEQ ID |
| | AfUmGmCmUmGfAmUfG | NO: 680 | UGCCCCCC | NO: 280 |
| | mCmCmCmCmsCmsCm | | | |
| ETXS388 | GmsGfsGmCmCmUfCmAf | SEQ ID | GGGCCUCAGCAAAGU | SEQ ID |
| | GfCmAmAmAmGfUmAfA | NO: 681 | AAUACUCU | NO: 281 |
| | mUmAmCmUmsCmsUm | | | |
| ETXS390 | GmsAfsCmAmGmCfGmGf | SEQ ID | GACAGCGGCAUGAAC | SEQ ID |
| | CfAmUmGmAmAfCmCfC | NO: 682 | CCCACCGU | NO: 282 |
| | mCmAmCmCmsGmsUm | | | |
| ETXS392 | CmsAfsUmGmAmAfGmAf | SEQ ID | CAUGAAGAGCAGGCA | SEQ ID |
| | GfCmAmGmGmCfAmGfC | NO: 683 | GCUGGUGC | NO: 283 |
| | mUmGmGmUmsGmsCm | | | |
| ETXS394 | CmsAfsCmUmGmGfGmAf | SEQ ID | CACUGGGAAUUUAUU | SEQ ID |
| | AfUmUmUmAmUfUmCfA | NO: 684 | CACCCAGG | NO: 284 |
| | mCmCmCmAmsGmsGm | | | |
| ETXS396 | UmsUfsCmUmGmUfUmUf | SEQ ID | UUCUGUUUGUCAUGC | SEQ ID |
| | GfUmCmAmUmGfCmUfU | NO: 685 | UUUUUUGC | NO: 285 |
| | mUmUmUmUmsGmsCm | | | |
| ETXS398 | UmsCfsGmAmCmAfGmUf | SEQ ID | UCGACAGUGAAGCGG | SEQ ID |
| | GfAmAmGmCmGfGmAfC | NO: 686 | ACUUGGGU | NO: 286 |
| | mUmUmGmGmsGmsUm | | | |
| ETXS400 | AmsAfsGmAmCmGfCmUf | SEQ ID | AAGACGCUGGAUCCG | SEQ ID |
| | GfGmAmUmCmCfGmGfC | NO: 687 | GCUCUUGC | NO: 287 |
| | mUmCmUmUmsGmsCm | | | |
| ETXS402 | AmsGfsUmUmCmUfGmUf | SEQ ID | AGUUCUGUUUGUCAU | SEQ ID |
| | UfUmGmUmCmAfUmGfC | NO: 688 | GCUUUUUU | NO: 288 |
| | mUmUmUmUmsUmsUm | | | |
| ETXS404 | CmsUfsUmGmGmGfUmGf | SEQ ID | CUUGGGUGGACAGCG | SEQ ID |
| | GfAmCmAmGmCfGmGfC | NO: 689 | GCAUGAAC | NO: 289 |
| | mAmUmGmAmsAmsCm | | | |
| ETXS406 | UmsCfsGmAmGmUfUmCf | SEQ ID | UCGAGUUCUGUUUGU | SEQ ID |
| | UfGmUmUmUmGfUmCfA | NO: 690 | CAUGCUUU | NO: 290 |
| | mUmGmCmUmsUmsUm | | | |
| ETXS408 | GmsCfsCmGmGmAfAmGf | SEQ ID | GCCGGAAGUUGUGGU | SEQ ID |
| | UfUmGmUmGmGfUmUfG | NO: 691 | UGUGUGUC | NO: 291 |
| | mUmGmUmGmsUmsCm | | | |
| ETXS410 | CmsAfsGmCmCmGfGmAf | SEQ ID | CAGCCGGAAGUUGUG | SEQ ID |
| | AfGmUmUmGmUfGmGfU | NO: 692 | GUUGUGUG | NO: 292 |
| | mUmGmUmGmsUmsGm | | | |
| ETXS412 | UmsAfsAmGmAmCfGmCf | SEQ ID | UAAGACGCUGGAUCC | SEQ ID |
| | UfGmGmAmUmCfCmGfG | NO: 693 | GGCUCUUG | NO: 293 |
| | mCmUmCmUmsUmsGm | | | |
| ETXS414 | UmsCfsAmGmCmCfGmGf | SEQ ID | UCAGCCGGAAGUUGU | SEQ ID |
| | AfAmGmUmUmGfUmGfG | NO: 694 | GGUUGUGU | NO: 294 |
| | mUmUmGmUmsGmsUm | | | |
| ETXS416 | UmsUfsGmGmUmGfCmUf | SEQ ID | UUGGUGCUUGAACAG | SEQ ID |
| | UfGmAmAmCmAfGmGfU | NO: 695 | GUCGAUGG | NO: 295 |
| | mCmGmAmUmsGmsGm | | | |
| ETXS418 | UmsUfsUmUmGmCfCmAf | SEQ ID | UUUUGCCAUCUCUCC | SEQ ID |
| | UfCmUmCmUmCfCmAfC | NO: 696 | ACCACCCG | NO: 296 |
| | mCmAmCmCmsCmsGm | | | |
| ETXS420 | GmsAfsCmAmGmUfGmAf | SEQ ID | GACAGUGAAGCGGAC | SEQ ID |
| | AfGmCmGmGmAfCmUfU | NO: 697 | UUGGGUGG | NO: 297 |
| | mGmGmGmUmsGmsGm | | | |
| ETXS422 | UmsCfsUmCmGmAfGmUf | SEQ ID | UCUCGAGUUCUGUUU | SEQ ID |
| | UfCmUmGmUmUfUmGfU | NO: 698 | GUCAUGCU | NO: 298 |
| | mCmAmUmGmsCmsUm | | | |
| ETXS424 | UmsGfsUmUmAmAfGmAf | SEQ ID | UGUUAAGACGCUGGA | SEQ ID |
| | CfGmCmUmGmGfAmUfC | NO: 699 | UCCGGCUC | NO: 299 |
| | mCmGmGmCmsUmsCm | | | |
| ETXS426 | GmsAfsCmGmCmUfGmGf | SEQ ID | GACGCUGGAUCCGGC | SEQ ID |
| | AfUmCmCmGmGfCmUfC | NO: 700 | UCUUGCCA | NO: 300 |
| | mUmUmGmCmsCmsAm | | | |
| ETXS428 | AmsCfsGmCmUmGfGmAf | SEQ ID | ACGCUGGAUCCGGCU | SEQ ID |
| | UfCmCmGmGmCfUmCfU | NO: 701 | CUUGCCAU | NO: 301 |
| | mUmGmCmCmsAmsUm | | | |
| ETXS430 | UmsGfsGmAmCmAfGmCf | SEQ ID | UGGACAGCGGCAUGA | SEQ ID |
| | GfGmCmAmUmGfAmAfC | NO: 702 | ACCCCACC | NO: 302 |
| | mCmCmCmAmsCmsCm | | | |
| ETXS472 | UmsUfsUmCfCmAfCmUf | SEQ ID | UUUCCACUGGGAAUU | SEQ ID |
| | GfGmGmAmAmUfUmUfA | NO: 703 | UAUUCACC | NO: 203 |
| | mUmUmCmAmsCmsCm | | | |
| ETXS474 | GmsAfsUmCfCmUfUmUf | SEQ ID | GAUCCUUUGAAGUAG | SEQ ID |
| | GfAmAmGmUmAfGmAfU | NO: 704 | AUGCAGUU | NO: 204 |
| | mGmCmAmGmsUmsUm | | | |
| ETXS476 | AmsUfsCmCfUmUfUmGf | SEQ ID | AUCCUUUGAAGUAGA | SEQ ID |
| | AfAmGmUmAmGfAmUfG | NO: 705 | UGCAGUUG | NO: 205 |
| | mCmAmGmUmsUmsGm | | | |
| ETXS478 | AmsCfsUmUfGmUfUmCf | SEQ ID | ACUUGUUCAUGGGUC | SEQ ID |
| | AfUmGmGmGmUfCmUfC | NO: 706 | UCUCCCUU | NO: 206 |
| | mUmCmCmCmsUmsUm | | | |
| ETXS480 | UmsCfsCmAfCmUfGmGf | SEQ ID | UCCACUGGGAAUUUA | SEQ ID |
| | GfAmAmUmUmUfAmUfU | NO: 707 | UUCACCCA | NO: 207 |
| | mCmAmCmCmsCmsAm | | | |
| ETXS482 | UmsGfsUmAfCmCfCmAf | SEQ ID | UGUACCCAAAAUUCC | SEQ ID |
| | AfAmAmUmUmCfCmUfC | NO: 708 | UCCUGAAG | NO: 208 |
| | mCmUmGmAmsAmsGm | | | |
| ETXS484 | UmsUfsCmCfCmAfUmGf | SEQ ID | UUCCCAUGAAGAGCA | SEQ ID |
| | AfAmGmAmGmCfAmGfG | NO: 709 | GGCAGCUG | NO: 209 |
| | mCmAmGmCmsUmsGm | | | |
| ETXS486 | UmsCfsUmUfUmUfAmUf | SEQ ID | UCUUUUAUGAGGCCC | SEQ ID |
| | GfAmGmGmCmCfCmUfU | NO: 710 | UUGGUGAG | NO: 210 |
| | mGmGmUmGmsAmsGm | | | |
| ETXS488 | AmsAfsCmUfGmCfUmUf | SEQ ID | AACUGCUUCUGGAUA | SEQ ID |
| | CfUmGmGmAmUfAmUfA | NO: 711 | UAAAGGUC | NO: 211 |
| | mAmAmGmGmsUmsCm | | | |
| ETXS490 | CmsCfsUmUfUmGfAmAf | SEQ ID | CCUUUGAAGUAGAUG | SEQ ID |
| | GfUmAmGmAmUfGmCfA | NO: 712 | CAGUUGAG | NO: 212 |
| | mGmUmUmGmsAmsGm | | | |
| ETXS492 | UmsUfsAmAfGmAfCmGf | SEQ ID | UUAAGACGCUGGAUC | SEQ ID |
| | CfUmGmGmAmUfCmCfG | NO: 713 | CGGCUCUU | NO: 213 |
| | mGmCmUmCmsUmsUm | | | |
| ETXS494 | GmsAfsGmGfAmUfGmUf | SEQ ID | GAGGAUGUUAAGACG | SEQ ID |
| | UfAmAmGmAmCfGmCfU | NO: 714 | CUGGAUCC | NO: 214 |
| | mGmGmAmUmsCmsCm | | | |
| ETXS496 | UmsCfsUmGfUmUfUmGf | SEQ ID | UCUGUUUGUCAUGCU | SEQ ID |
| | UfCmAmUmGmCfUmUfU | NO: 715 | UUUUUGCC | NO: 215 |
| | mUmUmUmGmsCmsCm | | | |
| ETXS498 | GmsUfsGmUfAmCfCmCf | SEQ ID | GUGUACCCAAAAUUC | SEQ ID |
| | AfAmAmAmUmUfCmCfU | NO: 716 | CUCCUGAA | NO: 216 |
| | mCmCmUmGmsAmsAm | | | |
| ETXS500 | UmsUfsCmAfGmCfCmGf | SEQ ID | UUCAGCCGGAAGUUG | SEQ ID |
| | GfAmAmGmUmUfGmUfG | NO: 717 | UGGUUGUG | NO: 217 |
| | mGmUmUmGmsUmsGm | | | |
| ETXS502 | UmsGfsUmUfUmGfUmCf | SEQ ID | UGUUUGUCAUGCUUU | SEQ ID |
| | AfUmGmCmUmUfUmUfU | NO: 718 | UUUGCCAU | NO: 218 |
| | mUmGmCmCmsAmsUm | | | |
| ETXS504 | AmsUfsUmUfCmCfAmCf | SEQ ID | AUUUCCACUGGGAAU | SEQ ID |
| | UfGmGmGmAmAfUmUfU | NO: 719 | UUAUUCAC | NO: 219 |
| | mAmUmUmCmsAmsCm | | | |
| ETXS506 | AmsCfsAmAfUmUfAmGf | SEQ ID | ACAAUUAGCAUGCUG | SEQ ID |
| | CfAmUmGmCmUfGmAfU | NO: 720 | AUGCCCCC | NO: 220 |
| | mGmCmCmCmsCmsCm | | | |
| ETXS508 | GmsUfsUmCfUmGfUmUf | SEQ ID | GUUCUGUUUGUCAUG | SEQ ID |
| | UfGmUmCmAmUfGmCfU | NO: 721 | CUUUUUUG | NO: 221 |
| | mUmUmUmUmsUmsGm | | | |
| ETXS510 | GmsGfsCmCfUmCfAmGfC | SEQ ID | GGCCUCAGCAAAGUA | SEQ ID |
| | fAmAmAmGmUfAmAfUm | NO: 722 | AUACUCUC | NO: 222 |
| | AmCmUmCmsUmsCm | | | |
| ETXS512 | UmsUfsUmCfCmAfCmUf | SEQ ID | UUUCCACUGGGAAUU | SEQ ID |
| | GfGmGmAmAmUfUmUfA | NO: 723 | UAUUCACC | NO: 203 |
| | mUmUmCmAmsCmsCm | | | |
| ETXS514 | GmsAfsUmCfCmUfUmUf | SEQ ID | GAUCCUUUGAAGUAG | SEQ ID |
| | GfAmAmGmUmAfGmAfU | NO: 724 | AUGCAGUU | NO: 204 |
| | mGmCmAmGmsUmsUm | | | |
| ETXS516 | AmsUfsCmCfUmUfUmGf | SEQ ID | AUCCUUUGAAGUAGA | SEQ ID |
| | AfAmGmUmAmGfAmUfG | NO: 725 | UGCAGUUG | NO: 205 |
| | mCmAmGmUmsUmsGm | | | |
| ETXS518 | AmsCfsUmUfGmUfUmCf | SEQ ID | ACUUGUUCAUGGGUC | SEQ ID |
| | AfUmGmGmGmUfCmUfC | NO: 726 | UCUCCCUU | NO: 206 |
| | mUmCmCmCmsUmsUm | | | |
| ETXS520 | UmsCfsCmAfCmUfGmGf | SEQ ID | UCCACUGGGAAUUUA | SEQ ID |
| | GfAmAmUmUmUfAmUfU | NO: 727 | UUCACCCA | NO: 207 |
| | mCmAmCmCmsCmsAm | | | |
| ETXS522 | UmsGfsUmAfCmCfCmAf | SEQ ID | UGUACCCAAAAUUCC | SEQ ID |
| | AfAmAmUmUmCfCmUfC | NO: 728 | UCCUGAAG | NO: 208 |
| | mCmUmGmAmsAmsGm | | | |
| ETXS524 | UmsUfsCmCfCmAfUmGf | SEQ ID | UUCCCAUGAAGAGCA | SEQ ID |
| | AfAmGmAmGmCfAmGfG | NO: 729 | GGCAGCUG | NO: 209 |
| | mCmAmGmCmsUmsGm | | | |
| ETXS526 | UmsCfsUmUfUmUfAmUf | SEQ ID | UCUUUUAUGAGGCCC | SEQ ID |
| | GfAmGmGmCmCfCmUfU | NO: 730 | UUGGUGAG | NO: 210 |
| | mGmGmUmGmsAmsGm | | | |
| ETXS528 | AmsAfsCmUfGmCfUmUf | SEQ ID | AACUGCUUCUGGAUA | SEQ ID |
| | CfUmGmGmAmUfAmUfA | NO: 731 | UAAAGGUC | NO: 211 |
| | mAmAmGmGmsUmsCm | | | |
| ETXS530 | CmsCfsUmUfUmGfAmAf | SEQ ID | CCUUUGAAGUAGAUG | SEQ ID |
| | GfUmAmGmAmUfGmCfA | NO: 732 | CAGUUGAG | NO: 212 |
| | mGmUmUmGmsAmsGm | | | |
| ETXS532 | UmsUfsAmAfGmAfCmGf | SEQ ID | UUAAGACGCUGGAUC | SEQ ID |
| | CfUmGmGmAmUfCmCfG | NO: 733 | CGGCUCUU | NO: 213 |
| | mGmCmUmCmsUmsUm | | | |
| ETXS534 | GmsAfsGmGfAmUfGmUf | SEQ ID | GAGGAUGUUAAGACG | SEQ ID |
| | UfAmAmGmAmCfGmCfU | NO: 734 | CUGGAUCC | NO: 214 |
| | mGmGmAmUmsCmsCm | | | |
| ETXS536 | UmsCfsUmGfUmUfUmGf | SEQ ID | UCUGUUUGUCAUGCU | SEQ ID |
| | UfCmAmUmGmCfUmUfU | NO: 735 | UUUUUGCC | NO: 215 |
| | mUmUmUmGmsCmsCm | | | |
| ETXS538 | GmsUfsGmUfAmCfCmCf | SEQ ID | GUGUACCCAAAAUUC | SEQ ID |
| | AfAmAmAmUmUfCmCfU | NO: 736 | CUCCUGAA | NO: 216 |
| | mCmCmUmGmsAmsAm | | | |
| ETXS540 | UmsUfsCmAfGmCfCmGf | SEQ ID | UUCAGCCGGAAGUUG | SEQ ID |
| | GfAmAmGmUmUfGmUfG | NO: 737 | UGGUUGUG | NO: 217 |
| | mGmUmUmGmsUmsGm | | | |
| ETXS542 | UmsGfsUmUfUmGfUmCf | SEQ ID | UGUUUGUCAUGCUUU | SEQ ID |
| | AfUmGmCmUmUfUmUfU | NO: 738 | UUUGCCAU | NO: 218 |
| | mUmGmCmCmsAmsUm | | | |
| ETXS544 | AmsUfsUmUfCmCfAmCf | SEQ ID | AUUUCCACUGGGAAU | SEQ ID |
| | UfGmGmGmAmAfUmUfU | NO: 739 | UUAUUCAC | NO: 219 |
| | mAmUmUmCmsAmsCm | | | |
| ETXS546 | AmsCfsAmAfUmUfAmGf | SEQ ID | ACAAUUAGCAUGCUG | SEQ ID |
| | CfAmUmGmCmUfGmAfU | NO: 740 | AUGCCCCC | NO: 220 |
| | mGmCmCmCmsCmsCm | | | |
| ETXS548 | GmsUfsUmCfUmGfUmUf | SEQ ID | GUUCUGUUUGUCAUG | SEQ ID |
| | UfGmUmCmAmUfGmCfU | NO: 741 | CUUUUUUG | NO: 221 |
| | mUmUmUmUmsUmsGm | | | |
| ETXS550 | GmsGfsCmCfUmCfAmGfC | SEQ ID | GGCCUCAGCAAAGUA | SEQ ID |
| | fAmAmAmGmUfAmAfUm | NO: 742 | AUACUCUC | NO: 222 |
| | AmCmUmCmsUmsCm | | | |
| ETXS552 | UmsUfsUmCfCmAfCmUf | SEQ ID | UUUCCACUGGGAAUU | SEQ ID |
| | GfGmGmAmAmUfUmUfA | NO: 743 | UAUUCACC | NO: 203 |
| | mUmUmCmAmsCmsCm | | | |
| ETXS554 | GmsAfsUmCfCmUfUmUf | SEQ ID | GAUCCUUUGAAGUAG | SEQ ID |
| | GfAmAmGmUmAfGmAfU | NO: 744 | AUGCAGUU | NO: 204 |
| | mGmCmAmGmsUmsUm | | | |
| ETXS556 | AmsUfsCmCfUmUfUmGf | SEQ ID | AUCCUUUGAAGUAGA | SEQ ID |
| | AfAmGmUmAmGfAmUfG | NO: 745 | UGCAGUUG | NO: 205 |
| | mCmAmGmUmsUmsGm | | | |
| ETXS558 | AmsCfsUmUfGmUfUmCf | SEQ ID | ACUUGUUCAUGGGUC | SEQ ID |
| | AfUmGmGmGmUfCmUfC | NO: 746 | UCUCCCUU | NO: 206 |
| | mUmCmCmCmsUmsUm | | | |
| ETXS560 | UmsCfsCmAfCmUfGmGf | SEQ ID | UCCACUGGGAAUUUA | SEQ ID |
| | GfAmAmUmUmUfAmUfU | NO: 747 | UUCACCCA | NO: 207 |
| | mCmAmCmCmsCmsAm | | | |
| ETXS562 | UmsGfsUmAfCmCfCmAf | SEQ ID | UGUACCCAAAAUUCC | SEQ ID |
| | AfAmAmUmUmCfCmUfC | NO: 748 | UCCUGAAG | NO: 208 |
| | mCmUmGmAmsAmsGm | | | |
| ETXS564 | UmsUfsCmCfCmAfUmGf | SEQ ID | UUCCCAUGAAGAGCA | SEQ ID |
| | AfAmGmAmGmCfAmGfG | NO: 749 | GGCAGCUG | NO: 209 |
| | mCmAmGmCmsUmsGm | | | |
| ETXS566 | UmsCfsUmUfUmUfAmUf | SEQ ID | UCUUUUAUGAGGCCC | SEQ ID |
| | GfAmGmGmCmCfCmUfU | NO: 750 | | NO: 210 |
| | mGmGmUmGmsAmsGm | | UUGGUGAG | |
| ETXS568 | AmsAfsCmUfGmCfUmUf | SEQ ID | AACUGCUUCUGGAUA | SEQ ID |
| | CfUmGmGmAmUfAmUfA | NO: 751 | UAAAGGUC | NO: 211 |
| | mAmAmGmGmsUmsCm | | | |
| ETXS570 | CmsCfsUmUfUmGfAmAf | SEQ ID | CCUUUGAAGUAGAUG | SEQ ID |
| | GfUmAmGmAmUfGmCfA | NO: 752 | CAGUUGAG | NO: 212 |
| | mGmUmUmGmsAmsGm | | | |
| ETXS572 | UmsUfsAmAfGmAfCmGf | SEQ ID | UUAAGACGCUGGAUC | SEQ ID |
| | CfUmGmGmAmUfCmCfG | NO: 753 | CGGCUCUU | NO: 213 |
| | mGmCmUmCmsUmsUm | | | |
| ETXS574 | GmsAfsGmGfAmUfGmUf | SEQ ID | GAGGAUGUUAAGACG | SEQ ID |
| | UfAmAmGmAmCfGmCfU | NO: 754 | CUGGAUCC | NO: 214 |
| | mGmGmAmUmsCmsCm | | | |
| ETXS576 | UmsCfsUmGfUmUfUmGf | SEQ ID | UCUGUUUGUCAUGCU | SEQ ID |
| | UfCmAmUmGmCfUmUfU | NO: 755 | UUUUUGCC | NO: 215 |
| | mUmUmUmGmsCmsCm | | | |
| ETXS578 | GmsUfsGmUfAmCfCmCf | SEQ ID | GUGUACCCAAAAUUC | SEQ ID |
| | AfAmAmAmUmUfCmCfU | NO: 756 | CUCCUGAA | NO: 216 |
| | mCmCmUmGmsAmsAm | | | |
| ETXS580 | UmsUfsCmAfGmCfCmGf | SEQ ID | UUCAGCCGGAAGUUG | SEQ ID |
| | GfAmAmGmUmUfGmUfG | NO: 757 | UGGUUGUG | NO: 217 |
| | mGmUmUmGmsUmsGm | | | |
| ETXS582 | UmsGfsUmUfUmGfUmCf | SEQ ID | UGUUUGUCAUGCUUU | SEQ ID |
| | AfUmGmCmUmUfUmUfU | NO: 758 | UUUGCCAU | NO: 218 |
| | mUmGmCmCmsAmsUm | | | |
| ETXS584 | AmsUfsUmUfCmCfAmCf | SEQ ID | AUUUCCACUGGGAAU | SEQ ID |
| | UfGmGmGmAmAfUmUfU | NO: 759 | UUAUUCAC | NO: 219 |
| | mAmUmUmCmsAmsCm | | | |
| ETXS586 | AmsCfsAmAfUmUfAmGf | SEQ ID | ACAAUUAGCAUGCUG | SEQ ID |
| | CfAmUmGmCmUfGmAfU | NO: 760 | AUGCCCCC | NO: 220 |
| | mGmCmCmCmsCmsCm | | | |
| ETXS588 | GmsUfsUmCfUmGfUmUf | SEQ ID | GUUCUGUUUGUCAUG | SEQ ID |
| | UfGmUmCmAmUfGmCfU | NO: 761 | CUUUUUUG | NO: 221 |
| | mUmUmUmUmsUmsGm | | | |
| ETXS590 | GmsGfsCmCfUmCfAmGfC | SEQ ID | GGCCUCAGCAAAGUA | SEQ ID |
| | fAmAmAmGmUfAmAfUm | NO: 762 | AUACUCUC | NO: 222 |
| | AmCmUmCmsUmsCm | | | |
| ETXS592 | UmsUfsUmCfCmAfCmUm | SEQ ID | UUUCCACUGGGAAUU | SEQ ID |
| | GmGmGmAmAmUfUmUf | NO: 763 | UAUUCACC | NO: 203 |
| | AmUmUmCmAmsCmsCm | | | |
| ETXS594 | GmsAfsUmCfCmUfUmUm | SEQ ID | GAUCCUUUGAAGUAG | SEQ ID |
| | GmAmAmGmUmAfGmAf | NO: 764 | AUGCAGUU | NO: 204 |
| | UmGmCmAmGmsUmsUm | | | |
| ETXS596 | AmsUfsCmCfUmUfUmGm | SEQ ID | AUCCUUUGAAGUAGA | SEQ ID |
| | AmAmGmUmAmGfAmUf | NO: 765 | UGCAGUUG | NO: 205 |
| | GmCmAmGmUmsUmsGm | | | |
| ETXS598 | AmsCfsUmUfGmUfUmCm | SEQ ID | ACUUGUUCAUGGGUC | SEQ ID |
| | AmUmGmGmGmUfCmUf | NO: 766 | UCUCCCUU | NO: 206 |
| | CmUmCmCmCmsUmsUm | | | |
| ETXS600 | UmsCfsCmAfCmUfGmGm | SEQ ID | UCCACUGGGAAUUUA | SEQ ID |
| | GmAmAmUmUmUfAmUf | NO: 767 | UUCACCCA | NO: 207 |
| | UmCmAmCmCmsCmsAm | | | |
| ETXS602 | UmsGfsUmAfCmCfCmAm | SEQ ID | UGUACCCAAAAUUCC | SEQ ID |
| | AmAmAmUmUmCfCmUf | NO: 768 | UCCUGAAG | NO: 208 |
| | CmCmUmGmAmsAmsGm | | | |
| ETXS604 | UmsUfsCmCfCmAfUmGm | SEQ ID | UUCCCAUGAAGAGCA | SEQ ID |
| | AmAmGmAmGmCfAmGf | NO: 769 | GGCAGCUG | NO: 209 |
| | GmCmAmGmCmsUmsGm | | | |
| ETXS606 | UmsCfsUmUfUmUfAmUm | SEQ ID | UCUUUUAUGAGGCCC | SEQ ID |
| | GmAmGmGmCmCfCmUf | NO: 770 | UUGGUGAG | NO: 210 |
| | UmGmGmUmGmsAmsGm | | | |
| ETXS608 | AmsAfsCmUfGmCfUmUm | SEQ ID | AACUGCUUCUGGAUA | SEQ ID |
| | CmUmGmGmAmUfAmUf | NO: 771 | UAAAGGUC | NO: 211 |
| | AmAmAmGmGmsUmsCm | | | |
| ETXS610 | CmsCfsUmUfUmGfAmAm | SEQ ID | CCUUUGAAGUAGAUG | SEQ ID |
| | GmUmAmGmAmUfGmCf | NO: 772 | CAGUUGAG | NO: 212 |
| | AmGmUmUmGmsAmsGm | | | |
| ETXS612 | UmsUfsAmAfGmAfCmGm | SEQ ID | UUAAGACGCUGGAUC | SEQ ID |
| | CmUmGmGmAmUfCmCf | NO: 773 | CGGCUCUU | NO: 213 |
| | GmGmCmUmCmsUmsUm | | | |
| ETXS614 | GmsAfsGmGfAmUfGmUm | SEQ ID | GAGGAUGUUAAGACG | SEQ ID |
| | UmAmAmGmAmCfGmCf | NO: 774 | CUGGAUCC | NO: 214 |
| | UmGmGmAmUmsCmsCm | | | |
| ETXS616 | UmsCfsUmGfUmUfUmGm | SEQ ID | UCUGUUUGUCAUGCU | SEQ ID |
| | UmCmAmUmGmCfUmUf | NO: 775 | UUUUUGCC | NO: 215 |
| | UmUmUmUmGmsCmsCm | | | |
| ETXS618 | GmsUfsGmUfAmCfCmCm | SEQ ID | GUGUACCCAAAAUUC | SEQ ID |
| | AmAmAmAmUmUfCmCf | NO: 776 | CUCCUGAA | NO: 216 |
| | UmCmCmUmGmsAmsAm | | | |
| ETXS620 | UmsUfsCmAfGmCfCmGm | SEQ ID | UUCAGCCGGAAGUUG | SEQ ID |
| | GmAmAmGmUmUfGmUf | NO: 777 | UGGUUGUG | NO: 217 |
| | GmGmUmUmGmsUmsGm | | | |
| ETXS622 | UmsGfsUmUfUmGfUmCm | SEQ ID | UGUUUGUCAUGCUUU | SEQ ID |
| | AmUmGmCmUmUfUmUf | NO: 778 | UUUGCCAU | NO: 218 |
| | UmUmGmCmCmsAmsUm | | | |
| ETXS624 | AmsUfsUmUfCmCfAmCm | SEQ ID | AUUUCCACUGGGAAU | SEQ ID |
| | UmGmGmGmAmAfUmUf | NO: 779 | UUAUUCAC | NO: 219 |
| | UmAmUmUmCmsAmsCm | | | |
| ETXS626 | AmsCfsAmAfUmUfAmGm | SEQ ID | ACAAUUAGCAUGCUG | SEQ ID |
| | CmAmUmGmCmUfGmAf | NO: 780 | AUGCCCCC | NO: 220 |
| | UmGmCmCmCmsCmsCm | | | |
| ETXS628 | GmsUfsUmCfUmGfUmUm | SEQ ID | GUUCUGUUUGUCAUG | SEQ ID |
| | UmGmUmCmAmUfGmCf | NO: 781 | CUUUUUUG | NO: 221 |
| | UmUmUmUmUmsUmsGm | | | |
| ETXS630 | GmsGfsCmCfUmCfAmGm | SEQ ID | GGCCUCAGCAAAGUA | SEQ ID |
| | CmAmAmAmGmUfAmAf | NO: 782 | AUACUCUC | NO: 222 |
| | UmAmCmUmCmsUmsCm | | | |
| ETXS632 | AmsAfsAmUmCmCfUmUf | SEQ ID | AAAUCCUUGUGGAUG | SEQ ID |
| | GfUmGmGmAmUfGmAfA | NO: 783 | AAGGCAAA | NO: 303 |
| | mGmGmCmAmsAmsAm | | | |
| ETXS634 | GmsCfsCmUmCmAfUmGf | SEQ ID | GCCUCAUGGAGAUCU | SEQ ID |
| | GfAmGmAmUmCfUmUfU | NO: 784 | UUCGCAGC | NO: 304 |
| | mCmGmCmAmsGmsCm | | | |
| ETXS636 | UmsUfsCmUmCmCfAmUf | SEQ ID | UUCUCCAUGAGGACC | SEQ ID |
| | GfAmGmGmAmCfCmAfC | NO: 785 | ACCAGCAU | NO: 305 |
| | mCmAmGmCmsAmsUm | | | |
| ETXS638 | UmsGfsCmCmUmCfAmUf | SEQ ID | UGCCUCAUGGAGAUC | SEQ ID |
| | GfGmAmGmAmUfCmUfU | NO: 786 | UUUCGCAG | NO: 306 |
| | mUmCmGmCmsAmsGm | | | |
| ETXS640 | AmsGfsCmAmGmCfUmCf | SEQ ID | AGCAGCUCAUGCAUC | SEQ ID |
| | AfUmGmCmAmUfCmUfC | NO: 787 | UCAUACUU | NO: 307 |
| | mAmUmAmCmsUmsUm | | | |
| ETXS642 | UmsCfsAmGmGmAfUmUf | SEQ ID | UCAGGAUUAAUCUCA | SEQ ID |
| | AfAmUmCmUmCfAmUfC | NO: 788 | UCAAACAG | NO: 308 |
| | mAmAmAmCmsAmsGm | | | |
| ETXS644 | UmsUfsGmGmUmUfUmCf | SEQ ID | UUGGUUUCAGGAUUA | SEQ ID |
| | AfGmGmAmUmUfAmAfU | NO: 789 | AUCUCAUC | NO: 309 |
| | mCmUmCmAmsUmsCm | | | |
| ETXS646 | GmsUfsUmUmCmAfGmGf | SEQ ID | GUUUCAGGAUUAAUC | SEQ ID |
| | AfUmUmAmAmUfCmUfC | NO: 790 | UCAUCAAA | NO: 310 |
| | mAmUmCmAmsAmsAm | | | |
| ETXS648 | UmsGfsGmUmUmUfCmAf | SEQ ID | UGGUUUCAGGAUUAA | SEQ ID |
| | GfGmAmUmUmAfAmUfC | NO: 791 | UCUCAUCA | NO: 311 |
| | mUmCmAmUmsCmsAm | | | |
| ETXS650 | GmsGfsUmUmUmCfAmGf | SEQ ID | GGUUUCAGGAUUAAU | SEQ ID |
| | GfAmUmUmAmAfUmCfU | NO: 792 | CUCAUCAA | NO: 312 |
| | mCmAmUmCmsAmsAm | | | |
| ETXS652 | AmsGfsGmAmUmUfAmAf | SEQ ID | AGGAUUAAUCUCAUC | SEQ ID |
| | UfCmUmCmAmUfCmAfA | NO: 793 | AAACAGUU | NO: 313 |
| | mAmCmAmGmsUmsUm | | | |
| ETXS654 | UmsCfsCmUmUmGfUmGf | SEQ ID | UCCUUGUGGAUGAAG | SEQ ID |
| | GfAmUmGmAmAfGmGfC | NO: 794 | GCAAAACU | NO: 314 |
| | mAmAmAmAmsCmsUm | | | |
| ETXS656 | GmsUfsGmCmCmUfCmAf | SEQ ID | GUGCCUCAUGGAGAU | SEQ ID |
| | UfGmGmAmGmAfUmCfU | NO: 795 | CUUUCGCA | NO: 315 |
| | mUmUmCmGmsCmsAm | | | |
| ETXS658 | UmsCfsUmCmCmAfUmGf | SEQ ID | UCUCCAUGAGGACCA | SEQ ID |
| | AfGmGmAmCmCfAmCfC | NO: 796 | CCAGCAUG | NO: 316 |
| | mAmGmCmAmsUmsGm | | | |
| ETXS660 | CmsAfsAmAmAmUfCmCf | SEQ ID | CAAAAUCCUUGUGGA | SEQ ID |
| | UfUmGmUmGmGfAmUfG | NO: 797 | UGAAGGCA | NO: 317 |
| | mAmAmGmGmsCmsAm | | | |
| ETXS662 | CmsGfsUmGmCmCfUmCf | SEQ ID | CGUGCCUCAUGGAGA | SEQ ID |
| | AfUmGmGmAmGfAmUfC | NO: 798 | UCUUUCGC | NO: 318 |
| | mUmUmUmCmsGmsCm | | | |
| ETXS664 | AmsUfsCmAmAmAfAmUf | SEQ ID | AUCAAAAUCCUUGUG | SEQ ID |
| | CfCmUmUmGmUfGmGfA | NO: 799 | GAUGAAGG | NO: 319 |
| | mUmGmAmAmsGmsGm | | | |
| ETXS666 | UmsUfsCmAmGmGfAmUf | SEQ ID | UUCAGGAUUAAUCUC | SEQ ID |
| | UfAmAmUmCmUfCmAfU | NO: 800 | AUCAAACA | NO: 320 |
| | mCmAmAmAmsCmsAm | | | |
| ETXS668 | AmsAfsUmCmCmUfUmGf | SEQ ID | AAUCCUUGUGGAUGA | SEQ ID |
| | UfGmGmAmUmGfAmAfG | NO: 801 | AGGCAAAA | NO: 321 |
| | mGmCmAmAmsAmsAm | | | |
| ETXS670 | CmsCfsAmUmCmGfUmGf | SEQ ID | CCAUCGUGCCUCAUG | SEQ ID |
| | CfCmUmCmAmUfGmGfA | NO: 802 | GAGAUCUU | NO: 322 |
| | mGmAmUmCmsUmsUm | | | |
| ETXS672 | UmsCfsCmAmUmGfAmGf | SEQ ID | UCCAUGAGGACCACC | SEQ ID |
| | GfAmCmCmAmCfCmAfG | NO: 803 | AGCAUGGU | NO: 323 |
| | mCmAmUmGmsGmsUm | | | |
| ETXS674 | AmsUfsCmCmUmUfGmUf | SEQ ID | AUCCUUGUGGAUGAA | SEQ ID |
| | GfGmAmUmGmAfAmGfG | NO: 804 | GGCAAAAC | NO: 324 |
| | mCmAmAmAmsAmsCm | | | |
| ETXS676 | AmsAfsAmAmUmCfCmUf | SEQ ID | AAAAUCCUUGUGGAU | SEQ ID |
| | UfGmUmGmGmAfUmGfA | NO: 805 | GAAGGCAA | NO: 325 |
| | mAmGmGmCmsAmsAm | | | |
| ETXS678 | CmsAfsUmGmGmUfGmGf | SEQ ID | CAUGGUGGCAUUUCC | SEQ ID |
| | CfAmUmUmUmCfCmUfU | NO: 806 | UUGGUAGG | NO: 326 |
| | mGmGmUmAmsGmsGm | | | |
| ETXS680 | UmsUfsUmCmAmGfGmAf | SEQ ID | UUUCAGGAUUAAUCU | SEQ ID |
| | UfUmAmAmUmCfUmCfA | NO: 807 | CAUCAAAC | NO: 327 |
| | mUmCmAmAmsAmsCm | | | |
| ETXS682 | UmsCfsAmAmAmAfUmCf | SEQ ID | UCAAAAUCCUUGUGG | SEQ ID |
| | CfUmUmGmUmGfGmAfU | NO: 808 | AUGAAGGC | NO: 328 |
| | mGmAmAmGmsGmsCm | | | |
| ETXS684 | UmsCfsGmUmGmCfCmUf | SEQ ID | UCGUGCCUCAUGGAG | SEQ ID |
| | CfAmUmGmGmAfGmAfU | NO: 809 | AUCUUUCG | NO: 329 |
| | mCmUmUmUmsCmsGm | | | |
| ETXS686 | CmsAfsGmGmAmUfUmAf | SEQ ID | CAGGAUUAAUCUCAU | SEQ ID |
| | AfUmCmUmCmAfUmCfA | NO: 810 | CAAACAGU | NO: 330 |
| | mAmAmCmAmsGmsUm | | | |
| ETXS688 | CmsUfsCmCmAmUfGmAf | SEQ ID | CUCCAUGAGGACCAC | SEQ ID |
| | GfGmAmCmCmAfCmCfA | NO: 811 | CAGCAUGG | NO: 331 |
| | mGmCmAmUmsGmsGm | | | |
| ETXS690 | CmsCfsUmUmGmUfGmGf | SEQ ID | CCUUGUGGAUGAAGG | SEQ ID |
| | AfUmGmAmAmGfGmCfA | NO: 812 | CAAAACUC | NO: 332 |
| | mAmAmAmCmsUmsCm | | | |
| ETXS692 | UmsGfsAmGmGmAfCmCf | SEQ ID | UGAGGACCACCAGCA | SEQ ID |
| | AfCmCmAmGmCfAmUfG | NO: 813 | UGGUGGCA | NO: 333 |
| | mGmUmGmGmsCmsAm | | | |
| ETXS694 | AmsUfsGmAmGmGfAmCf | SEQ ID | AUGAGGACCACCAGC | SEQ ID |
| | CfAmCmCmAmGfCmAfU | NO: 814 | AUGGUGGC | NO: 334 |
| | mGmGmUmGmsGmsCm | | | |
| ETXS696 | AmsUfsCmGmUmGfCmCf | SEQ ID | AUCGUGCCUCAUGGA | SEQ ID |
| | UfCmAmUmGmGfAmGfA | NO: 815 | GAUCUUUC | NO: 335 |
| | mUmCmUmUmsUmsCm | | | |
| ETXS698 | CmsUfsUmGmUmGfGmAf | SEQ ID | CUUGUGGAUGAAGGC | SEQ ID |
| | UfGmAmAmGmGfCmAfA | NO: 816 | AAAACUCC | NO: 336 |
| | mAmAmCmUmsCmsCm | | | |
| ETXS700 | CmsAfsUmGmAmGfGmAf | SEQ ID | CAUGAGGACCACCAG | SEQ ID |
| | CfCmAmCmCmAfGmCfA | NO: 817 | CAUGGUGG | NO: 337 |
| | mUmGmGmUmsGmsGm | | | |
| ETXS702 | CmsAfsUmCmGmUfGmCf | SEQ ID | CAUCGUGCCUCAUGG | SEQ ID |
| | CfUmCmAmUmGfGmAfG | NO: 818 | AGAUCUUU | NO: 338 |
| | mAmUmCmUmsUmsUm | | | |
| ETXS704 | CmsCfsAmUmGmAfGmGf | SEQ ID | CCAUGAGGACCACCA | SEQ ID |
| | AfCmCmAmCmCfAmGfC | NO: 819 | GCAUGGUG | NO: 339 |
| | mAmUmGmGmsUmsGm | | | |
| ETXS706 | UmsUfsCmUmUmGfUmCf | SEQ ID | UUCUUGUCAAAGGUG | SEQ ID |
| | AfAmAmGmGmUfGmGfA | NO: 820 | GAGGCAAA | NO: 340 |
| | mGmGmCmAmsAmsAm | | | |
| ETXS708 | AmsAfsUmUmCmUfUmGf | SEQ ID | AAUUCUUGUCAAAGG | SEQ ID |
| | UfCmAmAmAmGfGmUfG | NO: 821 | UGGAGGCA | NO: 341 |
| | mGmAmGmGmsCmsAm | | | |
| ETXS710 | UmsAfsCmAmUmCfAmUf | SEQ ID | UACAUCAUGGGCACC | SEQ ID |
| | GfGmGmCmAmCfCmUfU | NO: 822 | UUAAUGGU | NO: 342 |
| | mAmAmUmGmsGmsUm | | | |
| ETXS712 | GmsGfsCmAmUmUfUmCf | SEQ ID | GGCAUUUCCUUGGUA | SEQ ID |
| | CfUmUmGmGmUfAmGfG | NO: 823 | GGGCAGUU | NO: 343 |
| | mGmCmAmGmsUmsUm | | | |
| ETXS714 | UmsUfsUmCmCmUfUmGf | SEQ ID | UUUCCUUGGUAGGGC | SEQ ID |
| | GfUmAmGmGmGfCmAfG | NO: 824 | AGUUUGAG | NO: 344 |
| | mUmUmUmGmsAmsGm | | | |
| ETXS716 | UmsUfsCmCmUmUfGmGf | SEQ ID | UUCCUUGGUAGGGCA | SEQ ID |
| | UfAmGmGmGmCfAmGfU | NO: 825 | GUUUGAGG | NO: 345 |
| | mUmUmGmAmsGmsGm | | | |
| ETXS718 | AmsAfsAmUmUmCfUmUf | SEQ ID | AAAUUCUUGUCAAAG | SEQ ID |
| | GfUmCmAmAmAfGmGfU | NO: 826 | GUGGAGGC | NO: 346 |
| | mGmGmAmGmsGmsCm | | | |
| ETXS720 | UmsUfsGmUmCmCfAmGf | SEQ ID | UUGUCCAGGUGGAAA | SEQ ID |
| | GfUmGmGmAmAfAmGfU | NO: 827 | GUGUCGAC | NO: 347 |
| | mGmUmCmGmsAmsCm | | | |
| ETXS722 | GmsUfsAmCmUmUfGmUf | SEQ ID | GUACUUGUCCAGGUG | SEQ ID |
| | CfCmAmGmGmUfGmGfA | NO: 828 | GAAAGUGU | NO: 348 |
| | mAmAmGmUmsGmsUm | | | |
| ETXS724 | AmsUfsUmUmCmCfUmUf | SEQ ID | AUUUCCUUGGUAGGG | SEQ ID |
| | GfGmUmAmGmGfGmCfA | NO: 829 | CAGUUUGA | NO: 349 |
| | mGmUmUmUmsGmsAm | | | |
| ETXS726 | GmsCfsAmUmUmUfCmCf | SEQ ID | GCAUUUCCUUGGUAG | SEQ ID |
| | UfUmGmGmUmAfGmGfG | NO: 830 | GGCAGUUU | NO: 350 |
| | mCmAmGmUmsUmsUm | | | |
| ETXS728 | UmsAfsCmUmUmGfUmCf | SEQ ID | UACUUGUCCAGGUGG | SEQ ID |
| | CfAmGmGmUmGfGmAfA | NO: 831 | AAAGUGUC | NO: 351 |
| | mAmGmUmGmsUmsCm | | | |
| ETXS730 | AmsCfsAmUmCmAfUmGf | SEQ ID | ACAUCAUGGGCACCU | SEQ ID |
| | GfGmCmAmCmCfUmUfA | NO: 832 | UAAUGGUC | NO: 352 |
| | mAmUmGmGmsUmsCm | | | |
| ETXS732 | UmsCfsUmUmGmUfCmAf | SEQ ID | UCUUGUCAAAGGUGG | SEQ ID |
| | AfAmGmGmUmGfGmAfG | NO: 833 | AGGCAAAC | NO: 353 |
| | mGmCmAmAmsAmsCm | | | |
| ETXS734 | UmsUfsGmUmAmCfUmUf | SEQ ID | UUGUACUUGUCCAGG | SEQ ID |
| | GfUmCmCmAmGfGmUfG | NO: 834 | UGGAAAGU | NO: 354 |
| | mGmAmAmAmsGmsUm | | | |
| ETXS736 | CmsUfsUmGmGmUfAmGf | SEQ ID | CUUGGUAGGGCAGUU | SEQ ID |
| | GfGmCmAmGmUfUmUfG | NO: 835 | UGAGGACA | NO: 355 |
| | mAmGmGmAmsCmsAm | | | |
| ETXS738 | CmsAfsUmUmUmCfCmUf | SEQ ID | CAUUUCCUUGGUAGG | SEQ ID |
| | UfGmGmUmAmGfGmGfC | NO: 836 | GCAGUUUG | NO: 356 |
| | mAmGmUmUmsUmsGm | | | |
| ETXS740 | CmsCfsUmUmGmGfUmAf | SEQ ID | CCUUGGUAGGGCAGU | SEQ ID |
| | GfGmGmCmAmGfUmUfU | NO: 837 | UUGAGGAC | NO: 357 |
| | mGmAmGmGmsAmsCm | | | |
| ETXS742 | UmsUfsGmGmUmAfGmGf | SEQ ID | UUGGUAGGGCAGUUU | SEQ ID |
| | GfCmAmGmUmUfUmGfA | NO: 838 | GAGGACAU | NO: 358 |
| | mGmGmAmCmsAmsUm | | | |
| ETXS744 | CmsUfsUmGmUmCfCmAf | SEQ ID | CUUGUCCAGGUGGAA | SEQ ID |
| | GfGmUmGmGmAfAmAfG | NO: 839 | AGUGUCGA | NO: 359 |
| | mUmGmUmCmsGmsAm | | | |
| ETXS746 | CmsUfsUmGmUmCfAmAf | SEQ ID | CUUGUCAAAGGUGGA | SEQ ID |
| | AfGmGmUmGmGfAmGfG | NO: 840 | GGCAAACU | NO: 360 |
| | mCmAmAmAmsCmsUm | | | |
| ETXS748 | AmsCfsUmUmGmUfCmCf | SEQ ID | ACUUGUCCAGGUGGA | SEQ ID |
| | AfGmGmUmGmGfAmAfA | NO: 841 | AAGUGUCG | NO: 361 |
| | mGmUmGmUmsCmsGm | | | |
| ETXS750 | CmsUfsUmGmUmAfCmUf | SEQ ID | CUUGUACUUGUCCAG | SEQ ID |
| | UfGmUmCmCmAfGmGfU | NO: 842 | GUGGAAAG | NO: 362 |
| | mGmGmAmAmsAmsGm | | | |
| ETXS752 | UmsGfsUmAmCmUfUmGf | SEQ ID | UGUACUUGUCCAGGU | SEQ ID |
| | UfCmCmAmGmGfUmGfG | NO: 843 | GGAAAGUG | NO: 363 |
| | mAmAmAmGmsUmsGm | | | |
| ETXS754 | UmsCfsCmUmUmGfGmUf | SEQ ID | UCCUUGGUAGGGCAG | SEQ ID |
| | AfGmGmGmCmAfGmUfU | NO: 844 | UUUGAGGA | NO: 364 |
| | mUmGmAmGmsGmsAm | | | |
| ETXS756 | UmsUfsCmCmCmUfUmUf | SEQ ID | UUCCCUUUGAACAAG | SEQ ID |
| | GfAmAmCmAmAfGmAfU | NO: 845 | AUGUAAUC | NO: 365 |
| | mGmUmAmAmsUmsCm | | | |
| ETXS758 | AmsGfsGmAmCmCfAmCf | SEQ ID | AGGACCACCAGCAUG | SEQ ID |
| | CfAmGmCmAmUfGmGfU | NO: 846 | GUGGCAUU | NO: 366 |
| | mGmGmCmAmsUmsUm | | | |
| ETXS760 | UmsAfsUmCmAmAfAmAf | SEQ ID | UAUCAAAAUACCUCU | SEQ ID |
| | UfAmCmCmUmCfUmUfG | NO: 847 | UGGAUAAA | NO: 367 |
| | mGmAmUmAmsAmsAm | | | |
| ETXS762 | AmsCfsCmAmCmCfAmGf | SEQ ID | ACCACCAGCAUGGUG | SEQ ID |
| | CfAmUmGmGmUfGmGfC | NO: 848 | GCAUUUCC | NO: 368 |
| | mAmUmUmUmsCmsCm | | | |
| ETXS764 | UmsCfsCmCmUmUfUmGf | SEQ ID | UCCCUUUGAACAAGA | SEQ ID |
| | AfAmCmAmAmGfAmUfG | NO: 849 | UGUAAUCC | NO: 369 |
| | mUmAmAmUmsCmsCm | | | |
| ETXS766 | UmsUfsGmCmCmAfUmCf | SEQ ID | UUGCCAUCGUGCCUC | SEQ ID |
| | GfUmGmCmCmUfCmAfU | NO: 850 | AUGGAGAU | NO: 370 |
| | mGmGmAmGmsAmsUm | | | |
| ETXS768 | UmsUfsUmGmAmUfGmAf | SEQ ID | UUUGAUGACAGGAGG | SEQ ID |
| | CfAmGmGmAmGfGmCfA | NO: 851 | CAUGGAAU | NO: 371 |
| | mUmGmGmAmsAmsUm | | | |
| ETXS770 | UmsGfsAmUmGmAfCmAf | SEQ ID | UGAUGACAGGAGGCA | SEQ ID |
| | GfGmAmGmGmCfAmUfG | NO: 852 | UGGAAUAA | NO: 372 |
| | mGmAmAmUmsAmsAm | | | |
| ETXS772 | CmsAfsGmCmAmUfGmGf | SEQ ID | CAGCAUGGUGGCAUU | SEQ ID |
| | UfGmGmCmAmUfUmUfC | NO: 853 | UCCUUGGU | NO: 373 |
| | mCmUmUmGmsGmsUm | | | |
| ETXS774 | UmsUfsUmUmCmUfCmCf | SEQ ID | UUUUCUCCAUGAGGA | SEQ ID |
| | AfUmGmAmGmGfAmCfC | NO: 854 | CCACCAGC | NO: 374 |
| | mAmCmCmAmsGmsCm | | | |
| ETXS776 | CmsCfsAmUmUmUfCmCf | SEQ ID | CCAUUUCCCUUUGAA | SEQ ID |
| | CfUmUmUmGmAfAmCfA | NO: 855 | CAAGAUGU | NO: 375 |
| | mAmGmAmUmsGmsUm | | | |
| ETXS778 | CmsCfsAmGmCmAfUmGf | SEQ ID | CCAGCAUGGUGGCAU | SEQ ID |
| | GfUmGmGmCmAfUmUfU | NO: 856 | UUCCUUGG | NO: 376 |
| | mCmCmUmUmsGmsGm | | | |
| ETXS780 | AmsUfsUmCmUmUfGmUf | SEQ ID | AUUCUUGUCAAAGGU | SEQ ID |
| | CfAmAmAmGmGfUmGfG | NO: 857 | GGAGGCAA | NO: 377 |
| | mAmGmGmCmsAmsAm | | | |
| ETXS782 | UmsUfsGmUmGmGfAmUf | SEQ ID | UUGUGGAUGAAGGCA | SEQ ID |
| | GfAmAmGmGmCfAmAfA | NO: 858 | AAACUCCC | NO: 378 |
| | mAmCmUmCmsCmsCm | | | |
| ETXS784 | CmsUfsCmAmUmGfGmAf | SEQ ID | CUCAUGGAGAUCUUU | SEQ ID |
| | GfAmUmCmUmUfUmCfG | NO: 859 | CGCAGCAG | NO: 379 |
| | mCmAmGmCmsAmsGm | | | |
| ETXS786 | UmsGfsUmCmAmAfAmGf | SEQ ID | UGUCAAAGGUGGAGG | SEQ ID |
| | GfUmGmGmAmGfGmCfA | NO: 860 | CAAACUUG | NO: 380 |
| | mAmAmCmUmsUmsGm | | | |
| ETXS788 | UmsUfsGmUmCmAfAmAf | SEQ ID | UUGUCAAAGGUGGAG | SEQ ID |
| | GfGmUmGmGmAfGmGfC | NO: 861 | GCAAACUU | NO: 381 |
| | mAmAmAmCmsUmsUm | | | |
| ETXS790 | CmsAfsUmUmUmCfCmCf | SEQ ID | CAUUUCCCUUUGAAC | SEQ ID |
| | UfUmUmGmAmAfCmAfA | NO: 862 | AAGAUGUA | NO: 382 |
| | mGmAmUmGmsUmsAm | | | |
| ETXS792 | UmsGfsUmGmGmAfUmGf | SEQ ID | UGUGGAUGAAGGCAA | SEQ ID |
| | AfAmGmGmCmAfAmAfA | NO: 863 | AACUCCCC | NO: 383 |
| | mCmUmCmCmsCmsCm | | | |
| ETXS794 | CmsCfsUmCmAmUfGmGf | SEQ ID | CCUCAUGGAGAUCUU | SEQ ID |
| | AfGmAmUmCmUfUmUfC | NO: 864 | UCGCAGCA | NO: 384 |
| | mGmCmAmGmsCmsAm | | | |
| ETXS796 | CmsUfsUmUmGmAfUmGf | SEQ ID | CUUUGAUGACAGGAG | SEQ ID |
| | AfCmAmGmGmAfGmGfC | NO: 865 | GCAUGGAA | NO: 385 |
| | mAmUmGmGmsAmsAm | | | |
| ETXS798 | UmsCfsAmCmCmAfCmCf | SEQ ID | UCACCACCCUGCCCA | SEQ ID |
| | CfUmGmCmCmCfAmGfA | NO: 866 | GAAACAGA | NO: 386 |
| | mAmAmCmAmsGmsAm | | | |
| ETXS800 | UmsGfsGmAmGmAfUmCf | SEQ ID | UGGAGAUCUUUCGCA | SEQ ID |
| | UfUmUmCmGmCfAmGfC | NO: 867 | GCAGGCUG | NO: 387 |
| | mAmGmGmCmsUmsGm | | | |
| ETXS802 | GmsAfsUmUmAmAfUmCf | SEQ ID | GAUUAAUCUCAUCAA | SEQ ID |
| | UfCmAmUmCmAfAmAfC | NO: 868 | ACAGUUUG | NO: 388 |
| | mAmGmUmUmsUmsGm | | | |
| ETXS804 | CmsAfsCmUmUmUfGmAf | SEQ ID | CACUUUGAUGACAGG | SEQ ID |
| | UfGmAmCmAmGfGmAfG | NO: 869 | AGGCAUGG | NO: 389 |
| | mGmCmAmUmsGmsGm | | | |
| ETXS806 | GmsCfsAmGmCmUfCmAf | SEQ ID | GCAGCUCAUGCAUCU | SEQ ID |
| | UfGmCmAmUmCfUmCfA | NO: 870 | CAUACUUC | NO: 390 |
| | mUmAmCmUmsUmsCm | | | |
| ETXS808 | CmsAfsGmCmUmCfAmUf | SEQ ID | CAGCUCAUGCAUCUC | SEQ ID |
| | GfCmAmUmCmUfCmAfU | NO: 871 | AUACUUCU | NO: 391 |
| | mAmCmUmUmsCmsUm | | | |
| ETXS810 | UmsGfsGmUmAmGfGmGf | SEQ ID | UGGUAGGGCAGUUUG | SEQ ID |
| | CfAmGmUmUmUfGmAfG | NO: 872 | AGGACAUG | NO: 392 |
| | mGmAmCmAmsUmsGm | | | |
| ETXS812 | GmsGfsAmUmUmAfAmUf | SEQ ID | GGAUUAAUCUCAUCA | SEQ ID |
| | CfUmCmAmUmCfAmAfA | NO: 873 | AACAGUUU | NO: 393 |
| | mCmAmGmUmsUmsUm | | | |
| ETXS814 | AmsUfsCmAmUmGfGmGf | SEQ ID | AUCAUGGGCACCUUA | SEQ ID |
| | CfAmCmCmUmUfAmAfU | NO: 874 | AUGGUCUU | NO: 394 |
| | mGmGmUmCmsUmsUm | | | |
| ETXS816 | CmsAfsUmCmAmUfGmGf | SEQ ID | CAUCAUGGGCACCUU | SEQ ID |
| | GfCmAmCmCmUfUmAfA | NO: 875 | AAUGGUCU | NO: 395 |
| | mUmGmGmUmsCmsUm | | | |
| ETXS818 | UmsUfsCmAmCmCfAmCf | SEQ ID | UUCACCACCCUGCCC | SEQ ID |
| | CfCmUmGmCmCfCmAfG | NO: 876 | AGAAACAG | NO: 396 |
| | mAmAmAmCmsAmsGm | | | |
| ETXS820 | CmsCfsAmCmCmCfUmGf | SEQ ID | CCACCCUGCCCAGAA | SEQ ID |
| | CfCmCmAmGmAfAmAfC | NO: 877 | ACAGAAGC | NO: 397 |
| | mAmGmAmAmsGmsCm | | | |
| ETXS822 | GmsCfsUmUmGmAfAmCf | SEQ ID | GCUUGAACUUCGGAA | SEQ ID |
| | UfUmCmGmGmAfAmAfG | NO: 878 | AGAAAACU | NO: 398 |
| | mAmAmAmAmsCmsUm | | | |
| ETXS824 | AmsGfsCmUmUmGfAmAf | SEQ ID | AGCUUGAACUUCGGA | SEQ ID |
| | CfUmUmCmGmGfAmAfA | NO: 879 | AAGAAAAC | NO: 399 |
| | mGmAmAmAmsAmsCm | | | |
| ETXS826 | AmsCfsCmAmCmCfCmUf | SEQ ID | ACCACCCUGCCCAGA | SEQ ID |
| | GfCmCmCmAmGfAmAfA | NO: 880 | AACAGAAG | NO: 400 |
| | mCmAmGmAmsAmsGm | | | |
| ETXS828 | GmsGfsUmAmGmGfGmCf | SEQ ID | GGUAGGGCAGUUUGA | SEQ ID |
| | AfGmUmUmUmGfAmGfG | NO: 881 | GGACAUGA | NO: 401 |
| | mAmCmAmUmsGmsAm | | | |
| ETXS830 | UmsGfsUmCmCmAfGmGf | SEQ ID | UGUCCAGGUGGAAAG | SEQ ID |
| | UfGmGmAmAmAfGmUfG | NO: 882 | UGUCGACU | NO: 402 |
| | mUmCmGmAmsCmsUm | | | |
| ETXS872 | AmsAfsAmUfCmCfUmUf | SEQ ID | AAAUCCUUGUGGAUG | SEQ ID |
| | GfUmGmGmAmUfGmAfA | NO: 883 | AAGGCAAA | NO: 303 |
| | mGmGmCmAmsAmsAm | | | |
| ETXS874 | GmsCfsCmUfCmAfUmGf | SEQ ID | GCCUCAUGGAGAUCU | SEQ ID |
| | GfAmGmAmUmCfUmUfU | NO: 884 | UUCGCAGC | NO: 304 |
| | mCmGmCmAmsGmsCm | | | |
| ETXS876 | UmsUfsCmUfCmCfAmUf | SEQ ID | UUCUCCAUGAGGACC | SEQ ID |
| | GfAmGmGmAmCfCmAfC | NO: 885 | ACCAGCAU | NO: 305 |
| | mCmAmGmCmsAmsUm | | | |
| ETXS878 | UmsGfsCmCfUmCfAmUf | SEQ ID | UGCCUCAUGGAGAUC | SEQ ID |
| | GfGmAmGmAmUfCmUfU | NO: 886 | UUUCGCAG | NO: 306 |
| | mUmCmGmCmsAmsGm | | | |
| ETXS880 | AmsGfsCmAfGmCfUmCf | SEQ ID | AGCAGCUCAUGCAUC | SEQ ID |
| | AfUmGmCmAmUfCmUfC | NO: 887 | UCAUACUU | NO: 307 |
| | mAmUmAmCmsUmsUm | | | |
| ETXS882 | UmsCfsAmGfGmAfUmUf | SEQ ID | UCAGGAUUAAUCUCA | SEQ ID |
| | AfAmUmCmUmCfAmUfC | NO: 888 | UCAAACAG | NO: 308 |
| | mAmAmAmCmsAmsGm | | | |
| ETXS884 | UmsUfsGmGfUmUfUmCf | SEQ ID | UUGGUUUCAGGAUUA | SEQ ID |
| | AfGmGmAmUmUfAmAfU | NO: 889 | AUCUCAUC | NO: 309 |
| | mCmUmCmAmsUmsCm | | | |
| ETXS886 | GmsUfsUmUfCmAfGmGf | SEQ ID | GUUUCAGGAUUAAUC | SEQ ID |
| | AfUmUmAmAmUfCmUfC | NO: 890 | UCAUCAAA | NO: 310 |
| | mAmUmCmAmsAmsAm | | | |
| ETXS888 | UmsGfsGmUfUmUfCmAf | SEQ ID | UGGUUUCAGGAUUAA | SEQ ID |
| | GfGmAmUmUmAfAmUfC | NO: 891 | UCUCAUCA | NO: 311 |
| | mUmCmAmUmsCmsAm | | | |
| ETXS890 | GmsGfsUmUfUmCfAmGf | SEQ ID | GGUUUCAGGAUUAAU | SEQ ID |
| | GfAmUmUmAmAfUmCfU | NO: 892 | CUCAUCAA | NO: 312 |
| | mCmAmUmCmsAmsAm | | | |
| ETXS892 | AmsGfsGmAfUmUfAmAf | SEQ ID | AGGAUUAAUCUCAUC | SEQ ID |
| | UfCmUmCmAmUfCmAfA | NO: 893 | AAACAGUU | NO: 313 |
| | mAmCmAmGmsUmsUm | | | |
| ETXS894 | UmsCfsCmUfUmGfUmGf | SEQ ID | UCCUUGUGGAUGAAG | SEQ ID |
| | GfAmUmGmAmAfGmGfC | NO: 894 | GCAAAACU | NO: 314 |
| | mAmAmAmAmsCmsUm | | | |
| ETXS896 | GmsUfsGmCfCmUfCmAf | SEQ ID | GUGCCUCAUGGAGAU | SEQ ID |
| | UfGmGmAmGmAfUmCfU | NO: 895 | CUUUCGCA | NO: 315 |
| | mUmUmCmGmsCmsAm | | | |
| ETXS898 | UmsCfsUmCfCmAfUmGf | SEQ ID | UCUCCAUGAGGACCA | SEQ ID |
| | AfGmGmAmCmCfAmCfC | NO: 896 | CCAGCAUG | NO: 316 |
| | mAmGmCmAmsUmsGm | | | |
| ETXS900 | CmsAfsAmAfAmUfCmCf | SEQ ID | CAAAAUCCUUGUGGA | SEQ ID |
| | UfUmGmUmGmGfAmUfG | NO: 897 | UGAAGGCA | NO: 317 |
| | mAmAmGmGmsCmsAm | | | |
| ETXS902 | CmsGfsUmGfCmCfUmCfA | SEQ ID | CGUGCCUCAUGGAGA | SEQ ID |
| | fUmGmGmAmGfAmUfCm | NO: 898 | UCUUUCGC | NO: 318 |
| | UmUmUmCmsGmsCm | | | |
| ETXS904 | AmsUfsCmAfAmAfAmUf | SEQ ID | AUCAAAAUCCUUGUG | SEQ ID |
| | CfCmUmUmGmUfGmGfA | NO: 899 | GAUGAAGG | NO: 319 |
| | mUmGmAmAmsGmsGm | | | |
| ETXS906 | UmsUfsCmAfGmGfAmUf | SEQ ID | UUCAGGAUUAAUCUC | SEQ ID |
| | UfAmAmUmCmUfCmAfU | NO: 900 | AUCAAACA | NO: 320 |
| | mCmAmAmAmsCmsAm | | | |
| ETXS908 | AmsAfsUmCfCmUfUmGf | SEQ ID | AAUCCUUGUGGAUGA | SEQ ID |
| | UfGmGmAmUmGfAmAfG | NO: 901 | AGGCAAAA | NO: 321 |
| | mGmCmAmAmsAmsAm | | | |
| ETXS910 | CmsCfsAmUfCmGfUmGfC | SEQ ID | CCAUCGUGCCUCAUG | SEQ ID |
| | fCmUmCmAmUfGmGfAm | NO: 902 | GAGAUCUU | NO: 322 |
| | GmAmUmCmsUmsUm | | | |
| ETXS912 | AmsAfsAmUfCmCfUmUf | SEQ ID | AAAUCCUUGUGGAUG | SEQ ID |
| | GfUmGmGmAmUfGmAfA | NO: 903 | AAGGCAAA | NO: 303 |
| | mGmGmCmAmsAmsAm | | | |
| ETXS914 | GmsCfsCmUfCmAfUmGf | SEQ ID | GCCUCAUGGAGAUCU | SEQ ID |
| | GfAmGmAmUmCfUmUfU | NO: 904 | UUCGCAGC | NO: 304 |
| | mCmGmCmAmsGmsCm | | | |
| ETXS916 | UmsUfsCmUfCmCfAmUf | SEQ ID | UUCUCCAUGAGGACC | SEQ ID |
| | GfAmGmGmAmCfCmAfC | NO: 905 | ACCAGCAU | NO: 305 |
| | mCmAmGmCmsAmsUm | | | |
| ETXS918 | UmsGfsCmCfUmCfAmUf | SEQ ID | UGCCUCAUGGAGAUC | SEQ ID |
| | GfGmAmGmAmUfCmUfU | NO: 906 | UUUCGCAG | NO: 306 |
| | mUmCmGmCmsAmsGm | | | |
| ETXS920 | AmsGfsCmAfGmCfUmCf | SEQ ID | AGCAGCUCAUGCAUC | SEQ ID |
| | AfUmGmCmAmUfCmUfC | NO: 907 | UCAUACUU | NO: 307 |
| | mAmUmAmCmsUmsUm | | | |
| ETXS922 | UmsCfsAmGfGmAfUmUf | SEQ ID | UCAGGAUUAAUCUCA | SEQ ID |
| | AfAmUmCmUmCfAmUfC | NO: 908 | UCAAACAG | NO: 308 |
| | mAmAmAmCmsAmsGm | | | |
| ETXS924 | UmsUfsGmGfUmUfUmCf | SEQ ID | UUGGUUUCAGGAUUA | SEQ ID |
| | AfGmGmAmUmUfAmAfU | NO: 909 | AUCUCAUC | NO: 309 |
| | mCmUmCmAmsUmsCm | | | |
| ETXS926 | GmsUfsUmUfCmAfGmGf | SEQ ID | GUUUCAGGAUUAAUC | SEQ ID |
| | AfUmUmAmAmUfCmUfC | NO: 910 | UCAUCAAA | NO: 310 |
| | mAmUmCmAmsAmsAm | | | |
| ETXS928 | UmsGfsGmUfUmUfCmAf | SEQ ID | UGGUUUCAGGAUUAA | SEQ ID |
| | GfGmAmUmUmAfAmUfC | NO: 911 | UCUCAUCA | NO: 311 |
| | mUmCmAmUmsCmsAm | | | |
| ETXS930 | GmsGfsUmUfUmCfAmGf | SEQ ID | GGUUUCAGGAUUAAU | SEQ ID |
| | GfAmUmUmAmAfUmCfU | NO: 912 | CUCAUCAA | NO: 312 |
| | mCmAmUmCmsAmsAm | | | |
| ETXS932 | AmsGfsGmAfUmUfAmAf | SEQ ID | AGGAUUAAUCUCAUC | SEQ ID |
| | UfCmUmCmAmUfCmAfA | NO: 913 | AAACAGUU | NO: 313 |
| | mAmCmAmGmsUmsUm | | | |
| ETXS934 | UmsCfsCmUfUmGfUmGf | SEQ ID | UCCUUGUGGAUGAAG | SEQ ID |
| | GfAmUmGmAmAfGmGfC | NO: 914 | GCAAAACU | NO: 314 |
| | mAmAmAmAmsCmsUm | | | |
| ETXS936 | GmsUfsGmCfCmUfCmAf | SEQ ID | GUGCCUCAUGGAGAU | SEQ ID |
| | UfGmGmAmGmAfUmCfU | NO: 915 | CUUUCGCA | NO: 315 |
| | mUmUmCmGmsCmsAm | | | |
| ETXS938 | UmsCfsUmCfCmAfUmGf | SEQ ID | UCUCCAUGAGGACCA | SEQ ID |
| | AfGmGmAmCmCfAmCfC | NO: 916 | CCAGCAUG | NO: 316 |
| | mAmGmCmAmsUmsGm | | | |
| ETXS940 | CmsAfsAmAfAmUfCmCf | SEQ ID | CAAAAUCCUUGUGGA | SEQ ID |
| | UfUmGmUmGmGfAmUfG | NO: 917 | UGAAGGCA | NO: 317 |
| | mAmAmGmGmsCmsAm | | | |
| ETXS942 | CmsGfsUmGfCmCfUmCfA | SEQ ID | CGUGCCUCAUGGAGA | SEQ ID |
| | fUmGmGmAmGfAmUfCm | NO: 918 | UCUUUCGC | NO: 318 |
| | UmUmUmCmsGmsCm | | | |
| ETXS944 | AmsUfsCmAfAmAfAmUf | SEQ ID | AUCAAAAUCCUUGUG | SEQ ID |
| | CfCmUmUmGmUfGmGfA | NO: 919 | GAUGAAGG | NO: 319 |
| | mUmGmAmAmsGmsGm | | | |
| ETXS946 | UmsUfsCmAfGmGfAmUf | SEQ ID | UUCAGGAUUAAUCUC | SEQ ID |
| | UfAmAmUmCmUfCmAfU | NO: 920 | AUCAAACA | NO: 320 |
| | mCmAmAmAmsCmsAm | | | |
| ETXS948 | AmsAfsUmCfCmUfUmGf | SEQ ID | AAUCCUUGUGGAUGA | SEQ ID |
| | UfGmGmAmUmGfAmAfG | NO: 921 | AGGCAAAA | NO: 321 |
| | mGmCmAmAmsAmsAm | | | |
| ETXS950 | CmsCfsAmUfCmGfUmGfC | SEQ ID | CCAUCGUGCCUCAUG | SEQ ID |
| | fCmUmCmAmUfGmGfAm | NO: 922 | GAGAUCUU | NO: 322 |
| | GmAmUmCmsUmsUm | | | |
| ETXS952 | AmsAfsAmUfCmCfUmUf | SEQ ID | AAAUCCUUGUGGAUG | SEQ ID |
| | GfUmGmGmAmUfGmAfA | NO: 923 | AAGGCAAA | NO: 303 |
| | mGmGmCmAmsAmsAm | | | |
| ETXS954 | GmsCfsCmUfCmAfUmGf | SEQ ID | GCCUCAUGGAGAUCU | SEQ ID |
| | GfAmGmAmUmCfUmUfU | NO: 924 | UUCGCAGC | NO: 304 |
| | mCmGmCmAmsGmsCm | | | |
| ETXS956 | UmsUfsCmUfCmCfAmUf | SEQ ID | UUCUCCAUGAGGACC | SEQ ID |
| | GfAmGmGmAmCfCmAfC | NO: 925 | ACCAGCAU | NO: 305 |
| | mCmAmGmCmsAmsUm | | | |
| ETXS958 | UmsGfsCmCfUmCfAmUf | SEQ ID | UGCCUCAUGGAGAUC | SEQ ID |
| | GfGmAmGmAmUfCmUfU | NO: 926 | UUUCGCAG | NO: 306 |
| | mUmCmGmCmsAmsGm | | | |
| ETXS960 | AmsGfsCmAfGmCfUmCf | SEQ ID | AGCAGCUCAUGCAUC | SEQ ID |
| | AfUmGmCmAmUfCmUfC | NO: 927 | UCAUACUU | NO: 307 |
| | mAmUmAmCmsUmsUm | | | |
| ETXS962 | UmsCfsAmGfGmAfUmUf | SEQ ID | UCAGGAUUAAUCUCA | SEQ ID |
| | AfAmUmCmUmCfAmUfC | NO: 928 | UCAAACAG | NO: 308 |
| | mAmAmAmCmsAmsGm | | | |
| ETXS964 | UmsUfsGmGfUmUfUmCf | SEQ ID | UUGGUUUCAGGAUUA | SEQ ID |
| | AfGmGmAmUmUfAmAfU | NO: 929 | AUCUCAUC | NO: 309 |
| | mCmUmCmAmsUmsCm | | | |
| ETXS966 | GmsUfsUmUfCmAfGmGf | SEQ ID | GUUUCAGGAUUAAUC | SEQ ID |
| | AfUmUmAmAmUfCmUfC | NO: 930 | UCAUCAAA | NO: 310 |
| | mAmUmCmAmsAmsAm | | | |
| ETXS968 | UmsGfsGmUfUmUfCmAf | SEQ ID | UGGUUUCAGGAUUAA | SEQ ID |
| | GfGmAmUmUmAfAmUfC | NO: 931 | UCUCAUCA | NO: 311 |
| | mUmCmAmUmsCmsAm | | | |
| ETXS970 | GmsGfsUmUfUmCfAmGf | SEQ ID | GGUUUCAGGAUUAAU | SEQ ID |
| | GfAmUmUmAmAfUmCfU | NO: 932 | CUCAUCAA | NO: 312 |
| | mCmAmUmCmsAmsAm | | | |
| ETXS972 | AmsGfsGmAfUmUfAmAf | SEQ ID | AGGAUUAAUCUCAUC | SEQ ID |
| | UfCmUmCmAmUfCmAfA | NO: 933 | AAACAGUU | NO: 313 |
| | mAmCmAmGmsUmsUm | | | |
| ETXS974 | UmsCfsCmUfUmGfUmGf | SEQ ID | UCCUUGUGGAUGAAG | SEQ ID |
| | GfAmUmGmAmAfGmGfC | NO: 934 | GCAAAACU | NO: 314 |
| | mAmAmAmAmsCmsUm | | | |
| ETXS976 | GmsUfsGmCfCmUfCmAf | SEQ ID | GUGCCUCAUGGAGAU | SEQ ID |
| | UfGmGmAmGmAfUmCfU | NO: 935 | CUUUCGCA | NO: 315 |
| | mUmUmCmGmsCmsAm | | | |
| ETXS978 | UmsCfsUmCfCmAfUmGf | SEQ ID | UCUCCAUGAGGACCA | SEQ ID |
| | AfGmGmAmCmCfAmCfC | NO: 936 | CCAGCAUG | NO: 316 |
| | mAmGmCmAmsUmsGm | | | |
| ETXS980 | CmsAfsAmAfAmUfCmCf | SEQ ID | CAAAAUCCUUGUGGA | SEQ ID |
| | UfUmGmUmGmGfAmUfG | NO: 937 | UGAAGGCA | NO: 317 |
| | mAmAmGmGmsCmsAm | | | |
| ETXS982 | CmsGfsUmGfCmCfUmCfA | SEQ ID | CGUGCCUCAUGGAGA | SEQ ID |
| | fUmGmGmAmGfAmUfCm | NO: 938 | UCUUUCGC | NO: 318 |
| | UmUmUmCmsGmsCm | | | |
| ETXS984 | AmsUfsCmAfAmAfAmUf | SEQ ID | AUCAAAAUCCUUGUG | SEQ ID |
| | CfCmUmUmGmUfGmGfA | NO: 939 | GAUGAAGG | NO: 319 |
| | mUmGmAmAmsGmsGm | | | |
| ETXS986 | UmsUfsCmAfGmGfAmUf | SEQ ID | UUCAGGAUUAAUCUC | SEQ ID |
| | UfAmAmUmCmUfCmAfU | NO: 940 | AUCAAACA | NO: 320 |
| | mCmAmAmAmsCmsAm | | | |
| ETXS988 | AmsAfsUmCfCmUfUmGf | SEQ ID | AAUCCUUGUGGAUGA | SEQ ID |
| | UfGmGmAmUmGfAmAfG | NO: 941 | AGGCAAAA | NO: 321 |
| | mGmCmAmAmsAmsAm | | | |
| ETXS990 | CmsCfsAmUfCmGfUmGfC | SEQ ID | CCAUCGUGCCUCAUG | SEQ ID |
| | fCmUmCmAmUfGmGfAm | NO: 942 | GAGAUCUU | NO: 322 |
| | GmAmUmCmsUmsUm | | | |
| ETXS992 | AmsAfsAmUfCmCfUmUm | SEQ ID | AAAUCCUUGUGGAUG | SEQ ID |
| | GmUmGmGmAmUfGmAf | NO: 943 | AAGGCAAA | NO: 303 |
| | AmGmGmCmAmsAmsAm | | | |
| ETXS994 | GmsCfsCmUfCmAfUmGm | SEQ ID | GCCUCAUGGAGAUCU | SEQ ID |
| | GmAmGmAmUmCfUmUf | NO: 944 | UUCGCAGC | NO: 304 |
| | UmCmGmCmAmsGmsCm | | | |
| ETXS996 | UmsUfsCmUfCmCfAmUm | SEQ ID | UUCUCCAUGAGGACC | SEQ ID |
| | GmAmGmGmAmCfCmAf | NO: 945 | ACCAGCAU | NO: 305 |
| | CmCmAmGmCmsAmsUm | | | |
| ETXS998 | UmsGfsCmCfUmCfAmUm | SEQ ID | UGCCUCAUGGAGAUC | SEQ ID |
| | GmGmAmGmAmUfCmUf | NO: 946 | UUUCGCAG | NO: 306 |
| | UmUmCmGmCmsAmsGm | | | |
| ETXS1000 | AmsGfsCmAfGmCfUmCm | SEQ ID | AGCAGCUCAUGCAUC | SEQ ID |
| | AmUmGmCmAmUfCmUf | NO: 947 | UCAUACUU | NO: 307 |
| | CmAmUmAmCmsUmsUm | | | |
| ETXS1002 | UmsCfsAmGfGmAfUmUm | SEQ ID | UCAGGAUUAAUCUCA | SEQ ID |
| | AmAmUmCmUmCfAmUf | NO: 948 | UCAAACAG | NO: 308 |
| | CmAmAmAmCmsAmsGm | | | |
| ETXS1004 | UmsUfsGmGfUmUfUmCm | SEQ ID | UUGGUUUCAGGAUUA | SEQ ID |
| | AmGmGmAmUmUfAmAf | NO: 949 | AUCUCAUC | NO: 309 |
| | UmCmUmCmAmsUmsCm | | | |
| ETXS1006 | GmsUfsUmUfCmAfGmGm | SEQ ID | GUUUCAGGAUUAAUC | SEQ ID |
| | AmUmUmAmAmUfCmUf | NO: 950 | UCAUCAAA | NO: 310 |
| | CmAmUmCmAmsAmsAm | | | |
| ETXS1008 | UmsGfsGmUfUmUfCmAm | SEQ ID | UGGUUUCAGGAUUAA | SEQ ID |
| | GmGmAmUmUmAfAmUf | NO: 951 | UCUCAUCA | NO: 311 |
| | CmUmCmAmUmsCmsAm | | | |
| ETXS1010 | GmsGfsUmUfUmCfAmGm | SEQ ID | GGUUUCAGGAUUAAU | SEQ ID |
| | GmAmUmUmAmAfUmCf | NO: 952 | CUCAUCAA | NO: 312 |
| | UmCmAmUmCmsAmsAm | | | |
| ETXS1012 | AmsGfsGmAfUmUfAmAm | SEQ ID | AGGAUUAAUCUCAUC | SEQ ID |
| | UmCmUmCmAmUfCmAf | NO: 953 | AAACAGUU | NO: 313 |
| | AmAmCmAmGmsUmsUm | | | |
| ETXS1014 | UmsCfsCmUfUmGfUmGm | SEQ ID | UCCUUGUGGAUGAAG | SEQ ID |
| | GmAmUmGmAmAfGmGf | NO: 954 | GCAAAACU | NO: 314 |
| | CmAmAmAmAmsCmsUm | | | |
| ETXS1016 | GmsUfsGmCfCmUfCmAm | SEQ ID | GUGCCUCAUGGAGAU | SEQ ID |
| | UmGmGmAmGmAfUmCf | NO: 955 | CUUUCGCA | NO: 315 |
| | UmUmUmCmGmsCmsAm | | | |
| ETXS1018 | UmsCfsUmCfCmAfUmGm | SEQ ID | UCUCCAUGAGGACCA | SEQ ID |
| | AmGmGmAmCmCfAmCf | NO: 956 | CCAGCAUG | NO: 316 |
| | CmAmGmCmAmsUmsGm | | | |
| ETXS1020 | CmsAfsAmAfAmUfCmCm | SEQ ID | CAAAAUCCUUGUGGA | SEQ ID |
| | UmUmGmUmGmGfAmUf | NO: 957 | UGAAGGCA | NO: 317 |
| | GmAmAmGmGmsCmsAm | | | |
| ETXS1022 | CmsGfsUmGfCmCfUmCm | SEQ ID | CGUGCCUCAUGGAGA | SEQ ID |
| | AmUmGmGmAmGfAmUf | NO: 958 | UCUUUCGC | NO: 318 |
| | CmUmUmUmCmsGmsCm | | | |
| ETXS1024 | AmsUfsCmAfAmAfAmUm | SEQ ID | AUCAAAAUCCUUGUG | SEQ ID |
| | CmCmUmUmGmUfGmGf | NO: 959 | GAUGAAGG | NO: 319 |
| | AmUmGmAmAmsGmsGm | | | |
| ETXS1026 | UmsUfsCmAfGmGfAmUm | SEQ ID | UUCAGGAUUAAUCUC | SEQ ID |
| | UmAmAmUmCmUfCmAf | NO: 960 | AUCAAACA | NO: 320 |
| | UmCmAmAmAmsCmsAm | | | |
| ETXS1028 | AmsAfsUmCfCmUfUmGm | SEQ ID | AAUCCUUGUGGAUGA | SEQ ID |
| | UmGmGmAmUmGfAmAf | NO: 961 | AGGCAAAA | NO: 321 |
| | GmGmCmAmAmsAmsAm | | | |
| ETXS1030 | CmsCfsAmUfCmGfUmGm | SEQ ID | CCAUCGUGCCUCAUG | SEQ ID |
| | CmCmUmCmAmUfGmGf | NO: 962 | GAGAUCUU | NO: 322 |
| | AmGmAmUmCmsUmsUm | | | |

Table 25 provides the modified second (sense) sequences, together with the corresponding unmodified second (sense) sequences for siRNA oligonucleosides (targeting HCII and ZPI) according to the present invention as follows.

TABLE 25

| | | | Underlying Base | |
|---|---|---|---|---|
| | | | Sequence 5′→3′ | |
| | Modified Second | SEQ ID | (Shown as an | SEQ ID |
| Sense | (Sense) Strand | NO | Unmodified | NO |
| strand ID | 5′→3′ | (SS-mod) | Nucleoside Sequence) | (SS-unmod) |
| ETXS231 | UmsGmsAmAmUmAmAfA | SEQ ID | UGAAUAAAUUCCCA | SEQ ID |
| | mUfUfCfCmCmAmGmUmG | NO: 963 | GUGGAAA | NO: 403 |
| | mGmAmAmAm | | | |
| ETXS233 | CmsUmsGmCmAmUmCfU | SEQ ID | CUGCAUCUACUUCA | SEQ ID |
| | mAfCfUfUmCmAmAmAmG | NO: 964 | AAGGAUC | NO: 404 |
| | mGmAmUmCm | | | |
| ETXS235 | AmsCmsUmGmCmAmUfC | SEQ ID | ACUGCAUCUACUUC | SEQ ID |
| | mUfAfCfUmUmCmAmAmA | NO: 965 | AAAGGAU | NO: 405 |
| | mGmGmAmUm | | | |
| ETXS237 | GmsGmsGmAmGmAmGfA | SEQ ID | GGGAGAGACCCAUG | SEQ ID |
| | mCfCfCfAmUmGmAmAmC | NO: 966 | AACAAGU | NO: 406 |
| | mAmAmGmUm | | | |
| ETXS239 | GmsGmsUmGmAmAmUfA | SEQ ID | GGUGAAUAAAUUC | SEQ ID |
| | mAfAfUfUmCmCmCmAmG | NO: 967 | CCAGUGGA | NO: 407 |
| | mUmGmGmAm | | | |
| ETXS241 | UmsCmsAmGmGmAmGfG | SEQ ID | UCAGGAGGAAUUU | SEQ ID |
| | mAfAfUfUmUmUmGmGm | NO: 968 | UGGGUACA | NO: 408 |
| | GmUmAmCmAm | | | |
| ETXS243 | GmsCmsUmGmCmCmUfG | SEQ ID | GCUGCCUGCUCUUC | SEQ ID |
| | mCfUfCfUmUmCmAmUmG | NO: 969 | AUGGGAA | NO: 409 |
| | mGmGmAmAm | | | |
| ETXS245 | CmsAmsCmCmAmAmGfG | SEQ ID | CACCAAGGGCCUCA | SEQ ID |
| | mGfCfCfUmCmAmUmAmA | NO: 970 | UAAAAGA | NO: 410 |
| | mAmAmGmAm | | | |
| ETXS247 | CmsCmsUmUmUmAmUfA | SEQ ID | CCUUUAUAUCCAGA | SEQ ID |
| | mUfCfCfAmGmAmAmGmC | NO: 971 | AGCAGUU | NO: 411 |
| | mAmGmUmUm | | | |
| ETXS249 | CmsAmsAmCmUmGmCfA | SEQ ID | CAACUGCAUCUACU | SEQ ID |
| | mUfCfUfAmCmUmUmCmA | NO: 972 | UCAAAGG | NO: 412 |
| | mAmAmGmGm | | | |
| ETXS251 | GmsAmsGmCmCmGmGfA | SEQ ID | GAGCCGGAUCCAGC | SEQ ID |
| | mUfCfCfAmGmCmGmUmC | NO: 973 | GUCUUAA | NO: 413 |
| | mUmUmAmAm | | | |
| ETXS253 | AmsUmsCmCmAmGmCfG | SEQ ID | AUCCAGCGUCUUAA | SEQ ID |
| | mUfCfUfUmAmAmCmAmU | NO: 974 | CAUCCUC | NO: 414 |
| | mCmCmUmCm | | | |
| ETXS255 | CmsAmsAmAmAmAmAfG | SEQ ID | CAAAAAAGCAUGAC | SEQ ID |
| | mCfAfUfGmAmCmAmAmA | NO: 975 | AAACAGA | NO: 415 |
| | mCmAmGmAm | | | |
| ETXS257 | CmsAmsGmGmAmGmGfA | SEQ ID | CAGGAGGAAUUUU | SEQ ID |
| | mAfUfUfUmUmGmGmGm | NO: 976 | GGGUACAC | NO: 416 |
| | UmAmCmAmCm | | | |
| ETXS259 | CmsAmsAmCmCmAmCfA | SEQ ID | CAACCACAACUUCC | SEQ ID |
| | mAfCfUfUmCmCmGmGmC | NO: 977 | GGCUGAA | NO: 417 |
| | mUmGmAmAm | | | |
| ETXS261 | GmsGmsCmAmAmAmAfA | SEQ ID | GGCAAAAAAGCAU | SEQ ID |
| | mAfGfCfAmUmGmAmCmA | NO: 978 | GACAAACA | NO: 418 |
| | mAmAmCmAm | | | |
| ETXS263 | GmsAmsAmUmAmAmAfU | SEQ ID | GAAUAAAUUCCCAG | SEQ ID |
| | mUfCfCfCmAmGmUmGmG | NO: 979 | UGGAAAU | NO: 419 |
| | mAmAmAmUm | | | |
| ETXS265 | GmsGmsGmCmAmUmCfA | SEQ ID | GGGCAUCAGCAUGC | SEQ ID |
| | mGfCfAfUmGmCmUmAmA | NO: 980 | UAAUUGU | NO: 420 |
| | mUmUmGmUm | | | |
| ETXS267 | AmsAmsAmAmAmGmCfA | SEQ ID | AAAAAGCAUGACA | SEQ ID |
| | mUfGfAfCmAmAmAmCmA | NO: 981 | AACAGAAC | NO: 421 |
| | mGmAmAmCm | | | |
| ETXS269 | GmsAmsGmUmAmUmUfA | SEQ ID | GAGUAUUACUUUG | SEQ ID |
| | mCfUfUfUmGmCmUmGmA | NO: 982 | CUGAGGCC | NO: 422 |
| | mGmGmCmCm | | | |
| ETXS271 | UmsCmsCmUmUmAmGfG | SEQ ID | UCCUUAGGUCUGAA | SEQ ID |
| | mUfCfUfGmAmAmGmGm | NO: 983 | GGGAGAG | NO: 423 |
| | GmAmGmAmGm | | | |
| ETXS273 | CmsAmsUmCmGmAmCfC | SEQ ID | CAUCGACCUGUUCA | SEQ ID |
| | mUfGfUfUmCmAmAmGmC | NO: 984 | AGCACCA | NO: 424 |
| | mAmCmCmAm | | | |
| ETXS275 | UmsCmsAmCmCmAmAfG | SEQ ID | UCACCAAGGGCCUC | SEQ ID |
| | mGfGfCfCmUmCmAmUmA | NO: 985 | AUAAAAG | NO: 425 |
| | mAmAmAmGm | | | |
| ETXS277 | AmsAmsUmAmAmAmUfU | SEQ ID | AAUAAAUUCCCAGU | SEQ ID |
| | mCfCfCfAmGmUmGmGmA | NO: 986 | GGAAAUG | NO: 426 |
| | mAmAmUmGm | | | |
| ETXS279 | GmsCmsAmAmAmAmAfA | SEQ ID | GCAAAAAAGCAUG | SEQ ID |
| | mGfCfAfUmGmAmCmAmA | NO: 987 | ACAAACAG | NO: 427 |
| | mAmCmAmGm | | | |
| ETXS281 | UmsUmsCmCmGmGmCfU | SEQ ID | UUCCGGCUGAAUGA | SEQ ID |
| | mGfAfAfUmGmAmGmAm | NO: 988 | GAGAGAG | NO: 428 |
| | GmAmGmAmGm | | | |
| ETXS283 | GmsCmsAmUmGmAmCfA | SEQ ID | GCAUGACAAACAGA | SEQ ID |
| | mAfAfCfAmGmAmAmCmU | NO: 989 | ACUCGAG | NO: 429 |
| | mCmGmAmGm | | | |
| ETXS285 | CmsUmsGmCmUmCmUfU | SEQ ID | CUGCUCUUCAUGGG | SEQ ID |
| | mCfAfUfGmGmGmAmAm | NO: 990 | AAGAGUG | NO: 430 |
| | GmAmGmUmGm | | | |
| ETXS287 | AmsGmsAmGmUmAmUfU | SEQ ID | AGAGUAUUACUUU | SEQ ID |
| | mAfCfUfUmUmGmCmUmG | NO: 991 | GCUGAGGC | NO: 431 |
| | mAmGmGmCm | | | |
| ETXS289 | CmsUmsUmCmAmGmGfA | SEQ ID | CUUCAGGAGGAAU | SEQ ID |
| | mGfGfAfAmUmUmUmUm | NO: 992 | UUUGGGUA | NO: 432 |
| | GmGmGmUmAm | | | |
| ETXS291 | AmsAmsGmCmAmUmGfA | SEQ ID | AAGCAUGACAAACA | SEQ ID |
| | mCfAfAfAmCmAmGmAmA | NO: 993 | GAACUCG | NO: 433 |
| | mCmUmCmGm | | | |
| ETXS293 | GmsUmsGmAmAmUmAfA | SEQ ID | GUGAAUAAAUUCCC | SEQ ID |
| | mAfUfUfCmCmCmAmGmU | NO: 994 | AGUGGAA | NO: 434 |
| | mGmGmAmAm | | | |
| ETXS295 | GmsGmsAmUmCmCmAfG | SEQ ID | GGAUCCAGCGUCUU | SEQ ID |
| | mCfGfUfCmUmUmAmAmC | NO: 995 | AACAUCC | NO: 435 |
| | mAmUmCmCm | | | |
| ETXS297 | GmsAmsCmAmAmAmCfA | SEQ ID | GACAAACAGAACUC | SEQ ID |
| | mGfAfAfCmUmCmGmAmG | NO: 996 | GAGAAGU | NO: 436 |
| | mAmAmGmUm | | | |
| ETXS299 | AmsUmsCmUmCmAmGfA | SEQ ID | AUCUCAGACCAAAG | SEQ ID |
| | mCfCfAfAmAmGmGmAmU | NO: 997 | GAUCGCC | NO: 437 |
| | mCmGmCmCm | | | |
| ETXS301 | CmsAmsAmCmUmUmCfC | SEQ ID | CAACUUCCGGCUGA | SEQ ID |
| | mGfGfCfUmGmAmAmUm | NO: 998 | AUGAGAG | NO: 438 |
| | GmAmGmAmGm | | | |
| ETXS303 | UmsUmsCmAmGmGmAfG | SEQ ID | UUCAGGAGGAAUU | SEQ ID |
| | mGfAfAfUmUmUmUmGm | NO: 999 | UUGGGUAC | NO: 439 |
| | GmGmUmAmCm | | | |
| ETXS305 | GmsAmsGmAmGmUmAfU | SEQ ID | GAGAGUAUUACUU | SEQ ID |
| | mUfAfCfUmUmUmGmCmU | NO: 1000 | UGCUGAGG | NO: 440 |
| | mGmAmGmGm | | | |
| ETXS307 | CmsCmsAmCmAmAmCfU | SEQ ID | CCACAACUUCCGGC | SEQ ID |
| | mUfCfCfGmGmCmUmGmA | NO: 1001 | UGAAUGA | NO: 441 |
| | mAmUmGmAm | | | |
| ETXS309 | CmsGmsGmAmUmCmCfA | SEQ ID | CGGAUCCAGCGUCU | SEQ ID |
| | mGfCfGfUmCmUmUmAmA | NO: 1002 | UAACAUC | NO: 442 |
| | mCmAmUmCm | | | |
| ETXS311 | UmsCmsUmCmAmAmCfU | SEQ ID | UCUCAACUGCAUCU | SEQ ID |
| | mGfCfAfUmCmUmAmCmU | NO: 1003 | ACUUCAA | NO: 443 |
| | mUmCmAmAm | | | |
| ETXS313 | GmsCmsAmUmCmUmCfA | SEQ ID | GCAUCUCAGACCAA | SEQ ID |
| | mGfAfCfCmAmAmAmGmG | NO: 1004 | AGGAUCG | NO: 444 |
| | mAmUmCmGm | | | |
| ETXS315 | AmsAmsAmGmCmAmUfG | SEQ ID | AAAGCAUGACAAAC | SEQ ID |
| | mAfCfAfAmAmCmAmGmA | NO: 1005 | AGAACUC | NO: 445 |
| | mAmCmUmCm | | | |
| ETXS317 | UmsCmsUmGmGmAmGfA | SEQ ID | UCUGGAGAAUAUA | SEQ ID |
| | mAfUfAfUmAmGmAmCmC | NO: 1006 | GACCCUGC | NO: 446 |
| | mCmUmGmCm | | | |
| ETXS319 | AmsGmsAmGmAmGmUfA | SEQ ID | AGAGAGUAUUACU | SEQ ID |
| | mUfUfAfCmUmUmUmGmC | NO: 1007 | UUGCUGAG | NO: 447 |
| | mUmGmAmGm | | | |
| ETXS321 | CmsCmsGmGmCmUmGfA | SEQ ID | CCGGCUGAAUGAGA | SEQ ID |
| | mAfUfGfAmGmAmGmAm | NO: 1008 | GAGAGGU | NO: 448 |
| | GmAmGmGmUm | | | |
| ETXS323 | CmsCmsUmGmGmGmUfG | SEQ ID | CCUGGGUGAAUAA | SEQ ID |
| | mAfAfUfAmAmAmUmUm | NO: 1009 | AUUCCCAG | NO: 449 |
| | CmCmCmAmGm | | | |
| ETXS325 | CmsUmsUmAmGmGmUfC | SEQ ID | CUUAGGUCUGAAG | SEQ ID |
| | mUfGfAfAmGmGmGmAm | NO: 1010 | GGAGAGAC | NO: 450 |
| | GmAmGmAmCm | | | |
| ETXS327 | CmsUmsUmCmCmGmGfC | SEQ ID | CUUCCGGCUGAAUG | SEQ ID |
| | mUfGfAfAmUmGmAmGm | NO: 1011 | AGAGAGA | NO: 451 |
| | AmGmAmGmAm | | | |
| ETXS329 | UmsAmsAmAmUmUmCfC | SEQ ID | UAAAUUCCCAGUGG | SEQ ID |
| | mCfAfGfUmGmGmAmAm | NO: 1012 | AAAUGAC | NO: 452 |
| | AmUmGmAmCm | | | |
| ETXS331 | AmsAmsCmCmAmCmAfA | SEQ ID | AACCACAACUUCCG | SEQ ID |
| | mCfUfUfCmCmGmGmCmU | NO: 1013 | GCUGAAU | NO: 453 |
| | mGmAmAmUm | | | |
| ETXS333 | GmsGmsAmGmAmAmUfA | SEQ ID | GGAGAAUAUAGAC | SEQ ID |
| | mUfAfGfAmCmCmCmUmG | NO: 1014 | CCUGCUAC | NO: 454 |
| | mCmUmAmCm | | | |
| ETXS335 | CmsCmsAmUmCmGmAfC | SEQ ID | CCAUCGACCUGUUC | SEQ ID |
| | mCfUfGfUmUmCmAmAmG | NO: 1015 | AAGCACC | NO: 455 |
| | mCmAmCmCm | | | |
| ETXS337 | UmsUmsCmUmCmAmAfC | SEQ ID | UUCUCAACUGCAUC | SEQ ID |
| | mUfGfCfAmUmCmUmAmC | NO: 1016 | UACUUCA | NO: 456 |
| | mUmUmCmAm | | | |
| ETXS339 | UmsCmsCmAmGmCmGfU | SEQ ID | UCCAGCGUCUUAAC | SEQ ID |
| | mCfUfUfAmAmCmAmUmC | NO: 1017 | AUCCUCA | NO: 457 |
| | mCmUmCmAm | | | |
| ETXS341 | AmsGmsGmCmAmUmCfU | SEQ ID | AGGCAUCUCAGACC | SEQ ID |
| | mCfAfGfAmCmCmAmAmA | NO: 1018 | AAAGGAU | NO: 458 |
| | mGmGmAmUm | | | |
| ETXS343 | GmsCmsCmGmCmUmGfU | SEQ ID | GCCGCUGUCCACCC | SEQ ID |
| | mCfCfAfCmCmCmAmAmG | NO: 1019 | AAGUCCG | NO: 459 |
| | mUmCmCmGm | | | |
| ETXS345 | AmsUmsGmAmCmAmAfA | SEQ ID | AUGACAAACAGAAC | SEQ ID |
| | mCfAfGfAmAmCmUmCmG | NO: 1020 | UCGAGAA | NO: 460 |
| | mAmGmAmAm | | | |
| ETXS347 | GmsGmsGmGmUmUmCfA | SEQ ID | GGGGUUCAUGCCGC | SEQ ID |
| | mUfGfCfCmGmCmUmGmU | NO: 1021 | UGUCCAC | NO: 461 |
| | mCmCmAmCm | | | |
| ETXS349 | GmsAmsUmCmCmAmGfC | SEQ ID | GAUCCAGCGUCUUA | SEQ ID |
| | mGfUfCfUmUmAmAmCmA | NO: 1022 | ACAUCCU | NO: 462 |
| | mUmCmCmUm | | | |
| ETXS351 | CmsCmsCmAmAmGmUfC | SEQ ID | CCCAAGUCCGCUUC | SEQ ID |
| | mCfGfCfUmUmCmAmCmU | NO: 1023 | ACUGUCG | NO: 463 |
| | mGmUmCmGm | | | |
| ETXS353 | AmsCmsUmUmCmCmGfG | SEQ ID | ACUUCCGGCUGAAU | SEQ ID |
| | mCfUfGfAmAmUmGmAm | NO: 1024 | GAGAGAG | NO: 464 |
| | GmAmGmAmGm | | | |
| ETXS355 | UmsAmsUmUmAmCmUfU | SEQ ID | UAUUACUUUGCUG | SEQ ID |
| | mUfGfCfUmGmAmGmGmC | NO: 1025 | AGGCCCAG | NO: 465 |
| | mCmCmAmGm | | | |
| ETXS357 | CmsUmsGmGmAmGmAfA | SEQ ID | CUGGAGAAUAUAG | SEQ ID |
| | mUfAfUfAmGmAmCmCmC | NO: 1026 | ACCCUGCU | NO: 466 |
| | mUmGmCmUm | | | |
| ETXS359 | UmsGmsAmUmUmCmUfC | SEQ ID | UGAUUCUCAACUGC | SEQ ID |
| | mAfAfCfUmGmCmAmUmC | NO: 1027 | AUCUACU | NO: 467 |
| | mUmAmCmUm | | | |
| ETXS361 | UmsGmsCmCmGmCmUfG | SEQ ID | UGCCGCUGUCCACC | SEQ ID |
| | mUfCfCfAmCmCmCmAmA | NO: 1028 | CAAGUCC | NO: 468 |
| | mGmUmCmCm | | | |
| ETXS363 | CmsUmsCmAmAmCmUfG | SEQ ID | CUCAACUGCAUCUA | SEQ ID |
| | mCfAfUfCmUmAmCmUmU | NO: 1029 | CUUCAAA | NO: 469 |
| | mCmAmAmAm | | | |
| ETXS365 | AmsCmsCmAmCmAmAfC | SEQ ID | ACCACAACUUCCGG | SEQ ID |
| | mUfUfCfCmGmGmCmUmG | NO: 1030 | CUGAAUG | NO: 470 |
| | mAmAmUmGm | | | |
| ETXS367 | CmsGmsGmUmGmGmGfG | SEQ ID | CGGUGGGGUUCAU | SEQ ID |
| | mUfUfCfAmUmGmCmCmG | NO: 1031 | GCCGCUGU | NO: 471 |
| | mCmUmGmUm | | | |
| ETXS369 | UmsCmsCmGmGmCmUfG | SEQ ID | UCCGGCUGAAUGAG | SEQ ID |
| | mAfAfUfGmAmGmAmGm | NO: 1032 | AGAGAGG | NO: 472 |
| | AmGmAmGmGm | | | |
| ETXS371 | UmsCmsUmUmCmAmGfG | SEQ ID | UCUUCAGGAGGAA | SEQ ID |
| | mAfGfGfAmAmUmUmUm | NO: 1033 | UUUUGGGU | NO: 473 |
| | UmGmGmGmUm | | | |
| ETXS373 | GmsUmsAmUmUmAmCfU | SEQ ID | GUAUUACUUUGCU | SEQ ID |
| | mUfUfGfCmUmGmAmGm | NO: 1034 | GAGGCCCA | NO: 474 |
| | GmCmCmCmAm | | | |
| ETXS375 | GmsAmsUmUmCmUmCfA | SEQ ID | GAUUCUCAACUGCA | SEQ ID |
| | mAfCfUfGmCmAmUmCmU | NO: 1035 | UCUACUU | NO: 475 |
| | mAmCmUmUm | | | |
| ETXS377 | CmsAmsUmCmUmCmAfG | SEQ ID | CAUCUCAGACCAAA | SEQ ID |
| | mAfCfCfAmAmAmGmGmA | NO: 1036 | GGAUCGC | NO: 476 |
| | mUmCmGmCm | | | |
| ETXS379 | GmsGmsCmAmUmCmUfC | SEQ ID | GGCAUCUCAGACCA | SEQ ID |
| | mAfGfAfCmCmAmAmAmG | NO: 1037 | AAGGAUC | NO: 477 |
| | mGmAmUmCm | | | |
| ETXS381 | UmsCmsAmAmCmUmGfC | SEQ ID | UCAACUGCAUCUAC | SEQ ID |
| | mAfUfCfUmAmCmUmUmC | NO: 1038 | UUCAAAG | NO: 478 |
| | mAmAmAmGm | | | |
| ETXS383 | CmsAmsAmGmUmCmCfG | SEQ ID | CAAGUCCGCUUCAC | SEQ ID |
| | mCfUfUfCmAmCmUmGmU | NO: 1039 | UGUCGAC | NO: 479 |
| | mCmGmAmCm | | | |
| ETXS385 | GmsGmsGmGmCmAmUfC | SEQ ID | GGGGCAUCAGCAUG | SEQ ID |
| | mAfGfCfAmUmGmCmUmA | NO: 1040 | CUAAUUG | NO: 480 |
| | mAmUmUmGm | | | |
| ETXS387 | AmsGmsUmAmUmUmAfC | SEQ ID | AGUAUUACUUUGC | SEQ ID |
| | mUfUfUfGmCmUmGmAm | NO: 1041 | UGAGGCCC | NO: 481 |
| | GmGmCmCmCm | | | |
| ETXS389 | GmsGmsUmGmGmGmGfU | SEQ ID | GGUGGGGUUCAUG | SEQ ID |
| | mUfCfAfUmGmCmCmGmC | NO: 1042 | CCGCUGUC | NO: 482 |
| | mUmGmUmCm | | | |
| ETXS391 | AmsCmsCmAmGmCmUfG | SEQ ID | ACCAGCUGCCUGCU | SEQ ID |
| | mCfCfUfGmCmUmCmUmU | NO: 1043 | CUUCAUG | NO: 483 |
| | mCmAmUmGm | | | |
| ETXS393 | UmsGmsGmGmUmGmAfA | SEQ ID | UGGGUGAAUAAAU | SEQ ID |
| | mUfAfAfAmUmUmCmCmC | NO: 1044 | UCCCAGUG | NO: 484 |
| | mAmGmUmGm | | | |
| ETXS395 | AmsAmsAmAmAmAmGfC | SEQ ID | AAAAAAGCAUGAC | SEQ ID |
| | mAfUfGfAmCmAmAmAmC | NO: 1045 | AAACAGAA | NO: 485 |
| | mAmGmAmAm | | | |
| ETXS397 | CmsCmsAmAmGmUmCfC | SEQ ID | CCAAGUCCGCUUCA | SEQ ID |
| | mGfCfUfUmCmAmCmUmG | NO: 1046 | CUGUCGA | NO: 486 |
| | mUmCmGmAm | | | |
| ETXS399 | AmsAmsGmAmGmCmCfG | SEQ ID | AAGAGCCGGAUCCA | SEQ ID |
| | mGfAfUfCmCmAmGmCmG | NO: 1047 | GCGUCUU | NO: 487 |
| | mUmCmUmUm | | | |
| ETXS401 | AmsAmsAmAmGmCmAfU | SEQ ID | AAAAGCAUGACAA | SEQ ID |
| | mGfAfCfAmAmAmCmAmG | NO: 1048 | ACAGAACU | NO: 488 |
| | mAmAmCmUm | | | |
| ETXS403 | UmsCmsAmUmGmCmCfG | SEQ ID | UCAUGCCGCUGUCC | SEQ ID |
| | mCfUfGfUmCmCmAmCmC | NO: 1049 | ACCCAAG | NO: 489 |
| | mCmAmAmGm | | | |
| ETXS405 | AmsGmsCmAmUmGmAfC | SEQ ID | AGCAUGACAAACAG | SEQ ID |
| | mAfAfAfCmAmGmAmAmC | NO: 1050 | AACUCGA | NO: 490 |
| | mUmCmGmAm | | | |
| ETXS407 | CmsAmsCmAmCmAmAfC | SEQ ID | CACACAACCACAAC | SEQ ID |
| | mCfAfCfAmAmCmUmUmC | NO: 1051 | UUCCGGC | NO: 491 |
| | mCmGmGmCm | | | |
| ETXS409 | CmsAmsCmAmAmCmCfA | SEQ ID | CACAACCACAACUU | SEQ ID |
| | mCfAfAfCmUmUmCmCmG | NO: 1052 | CCGGCUG | NO: 492 |
| | mGmCmUmGm | | | |
| ETXS411 | AmsGmsAmGmCmCmGfG | SEQ ID | AGAGCCGGAUCCAG | SEQ ID |
| | mAfUfCfCmAmGmCmGmU | NO: 1053 | CGUCUUA | NO: 493 |
| | mCmUmUmAm | | | |
| ETXS413 | AmsCmsAmAmCmCmAfC | SEQ ID | ACAACCACAACUUC | SEQ ID |
| | mAfAfCfUmUmCmCmGmG | NO: 1054 | CGGCUGA | NO: 494 |
| | mCmUmGmAm | | | |
| ETXS415 | AmsUmsCmGmAmCmCfU | SEQ ID | AUCGACCUGUUCAA | SEQ ID |
| | mGfUfUfCmAmAmGmCmA | NO: 1055 | GCACCAA | NO: 495 |
| | mCmCmAmAm | | | |
| ETXS417 | GmsGmsUmGmGmUmGfG | SEQ ID | GGUGGUGGAGAGA | SEQ ID |
| | mAfGfAfGmAmUmGmGm | NO: 1056 | UGGCAAAA | NO: 496 |
| | CmAmAmAmAm | | | |
| ETXS419 | AmsCmsCmCmAmAmGfU | SEQ ID | ACCCAAGUCCGCUU | SEQ ID |
| | mCfCfGfCmUmUmCmAmC | NO: 1057 | CACUGUC | NO: 497 |
| | mUmGmUmCm | | | |
| ETXS421 | CmsAmsUmGmAmCmAfA | SEQ ID | CAUGACAAACAGAA | SEQ ID |
| | mAfCfAfGmAmAmCmUmC | NO: 1058 | CUCGAGA | NO: 498 |
| | mGmAmGmAm | | | |
| ETXS423 | GmsCmsCmGmGmAmUfC | SEQ ID | GCCGGAUCCAGCGU | SEQ ID |
| | mCfAfGfCmGmUmCmUmU | NO: 1059 | CUUAACA | NO: 499 |
| | mAmAmCmAm | | | |
| ETXS425 | GmsCmsAmAmGmAmGfC | SEQ ID | GCAAGAGCCGGAUC | SEQ ID |
| | mCfGfGfAmUmCmCmAmG | NO: 1060 | CAGCGUC | NO: 500 |
| | mCmGmUmCm | | | |
| ETXS427 | GmsGmsCmAmAmGmAfG | SEQ ID | GGCAAGAGCCGGAU | SEQ ID |
| | mCfCfGfGmAmUmCmCmA | NO: 1061 | CCAGCGU | NO: 501 |
| | mGmCmGmUm | | | |
| ETXS429 | UmsGmsGmGmGmUmUfC | SEQ ID | UGGGGUUCAUGCCG | SEQ ID |
| | mAfUfGfCmCmGmCmUmG | NO: 1062 | CUGUCCA | NO: 502 |
| | mUmCmCmAm | | | |
| ETXS471 | UmsGmsAmAmUmAmAfAf | SEQ ID | UGAAUAAAUUCCCA | SEQ ID |
| | UfUfCfCmCmAmGmUmGm | NO: 1063 | GUGGAAA | NO: 403 |
| | GmAfAmAm | | | |
| ETXS473 | CmsUmsGmCmAmUmCfUf | SEQ ID | CUGCAUCUACUUCA | SEQ ID |
| | AfCfUfUmCmAmAmAmGm | NO: 1064 | AAGGAUC | NO: 404 |
| | GmAfUmCm | | | |
| ETXS475 | AmsCmsUmGmCmAmUfCf | SEQ ID | ACUGCAUCUACUUC | SEQ ID |
| | UfAfCfUmUmCmAmAmAm | NO: 1065 | AAAGGAU | NO: 405 |
| | GmGfAmUm | | | |
| ETXS477 | GmsGmsGmAmGmAmGfAf | SEQ ID | GGGAGAGACCCAUG | SEQ ID |
| | CfCfCfAmUmGmAmAmCm | NO: 1066 | AACAAGU | NO: 406 |
| | AmAfGmUm | | | |
| ETXS479 | GmsGmsUmGmAmAmUfAf | SEQ ID | GGUGAAUAAAUUC | SEQ ID |
| | AfAfUfUmCmCmCmAmGm | NO: 1067 | CCAGUGGA | NO: 407 |
| | UmGfGmAm | | | |
| ETXS481 | UmsCmsAmGmGmAmGfGf | SEQ ID | UCAGGAGGAAUUU | SEQ ID |
| | AfAfUfUmUmUmGmGmG | NO: 1068 | UGGGUACA | NO: 408 |
| | mUmAfCmAm | | | |
| ETXS483 | GmsCmsUmGmCmCmUfGf | SEQ ID | GCUGCCUGCUCUUC | SEQ ID |
| | CfUfCfUmUmCmAmUmGm | NO: 1069 | AUGGGAA | NO: 409 |
| | GmGfAmAm | | | |
| ETXS485 | CmsAmsCmCmAmAmGfGf | SEQ ID | CACCAAGGGCCUCA | SEQ ID |
| | GfCfCfUmCmAmUmAmAm | NO: 1070 | UAAAAGA | NO: 410 |
| | AmAfGmAm | | | |
| ETXS487 | CmsCmsUmUmUmAmUfAf | SEQ ID | CCUUUAUAUCCAGA | SEQ ID |
| | UfCfCfAmGmAmAmGmCm | NO: 1071 | AGCAGUU | NO: 411 |
| | AmGfUmUm | | | |
| ETXS489 | CmsAmsAmCmUmGmCfAf | SEQ ID | CAACUGCAUCUACU | SEQ ID |
| | UfCfUfAmCmUmUmCmAm | NO: 1072 | UCAAAGG | NO: 412 |
| | AmAfGmGm | | | |
| ETXS491 | GmsAmsGmCmCmGmGfAf | SEQ ID | GAGCCGGAUCCAGC | SEQ ID |
| | UfCfCfAmGmCmGmUmCm | NO: 1073 | GUCUUAA | NO: 413 |
| | UmUfAmAm | | | |
| ETXS493 | AmsUmsCmCmAmGmCfGf | SEQ ID | AUCCAGCGUCUUAA | SEQ ID |
| | UfCfUfUmAmAmCmAmUm | NO: 1074 | CAUCCUC | NO: 414 |
| | CmCfUmCm | | | |
| ETXS495 | CmsAmsAmAmAmAmAfGf | SEQ ID | CAAAAAAGCAUGAC | SEQ ID |
| | CfAfUfGmAmCmAmAmAm | NO: 1075 | AAACAGA | NO: 415 |
| | CmAfGmAm | | | |
| ETXS497 | CmsAmsGmGmAmGmGfAf | SEQ ID | CAGGAGGAAUUUU | SEQ ID |
| | AfUfUfUmUmGmGmGmU | NO: 1076 | GGGUACAC | NO: 416 |
| | mAmCfAmCm | | | |
| ETXS499 | CmsAmsAmCmCmAmCfAf | SEQ ID | CAACCACAACUUCC | SEQ ID |
| | AfCfUfUmCmCmGmGmCm | NO: 1077 | GGCUGAA | NO: 417 |
| | UmGfAmAm | | | |
| ETXS501 | GmsGmsCmAmAmAmAfAf | SEQ ID | GGCAAAAAAGCAU | SEQ ID |
| | AfGfCfAmUmGmAmCmAm | NO: 1078 | GACAAACA | NO: 418 |
| | AmAfCmAm | | | |
| ETXS503 | GmsAmsAmUmAmAmAfUf | SEQ ID | GAAUAAAUUCCCAG | SEQ ID |
| | UfCfCfCmAmGmUmGmGm | NO: 1079 | UGGAAAU | NO: 419 |
| | AmAfAmUm | | | |
| ETXS505 | GmsGmsGmCmAmUmCfAf | SEQ ID | GGGCAUCAGCAUGC | SEQ ID |
| | GfCfAfUmGmCmUmAmAm | NO: 1080 | UAAUUGU | NO: 420 |
| | UmUfGmUm | | | |
| ETXS507 | AmsAmsAmAmAmGmCfAf | SEQ ID | AAAAAGCAUGACA | SEQ ID |
| | UfGfAfCmAmAmAmCmAm | NO: 1081 | AACAGAAC | NO: 421 |
| | GmAfAmCm | | | |
| ETXS509 | GmsAmsGmUmAmUmUfAf | SEQ ID | GAGUAUUACUUUG | SEQ ID |
| | CfUfUfUmGmCmUmGmAm | NO: 1082 | CUGAGGCC | NO: 422 |
| | GmGfCmCm | | | |
| ETXS511 | UmsGmsAmAmUmAfAfAm | SEQ ID | UGAAUAAAUUCCCA | SEQ ID |
| | UfUfCfCfCmAmGmUmGm | NO: 1083 | GUGGAAA | NO: 403 |
| | GmAmAmAm | | | |
| ETXS513 | CmsUmsGmCmAmUfCfUm | SEQ ID | CUGCAUCUACUUCA | SEQ ID |
| | AfCfUfUfCmAmAmAmGm | NO: 1084 | AAGGAUC | NO: 404 |
| | GmAmUmCm | | | |
| ETXS515 | AmsCmsUmGmCmAfUfCm | SEQ ID | ACUGCAUCUACUUC | SEQ ID |
| | UfAfCfUfUmCmAmAmAm | NO: 1085 | AAAGGAU | NO: 405 |
| | GmGmAmUm | | | |
| ETXS517 | GmsGmsGmAmGmAfGfAm | SEQ ID | GGGAGAGACCCAUG | SEQ ID |
| | CfCfCfAfUmGmAmAmCm | NO: 1086 | AACAAGU | NO: 406 |
| | AmAmGmUm | | | |
| ETXS519 | GmsGmsUmGmAmAfUfAm | SEQ ID | GGUGAAUAAAUUC | SEQ ID |
| | AfAfUfUfCmCmCmAmGm | NO: 1087 | CCAGUGGA | NO: 407 |
| | UmGmGmAm | | | |
| ETXS521 | UmsCmsAmGmGmAfGfGm | SEQ ID | UCAGGAGGAAUUU | SEQ ID |
| | AfAfUfUfUmUmGmGmGm | NO: 1088 | UGGGUACA | NO: 408 |
| | UmAmCmAm | | | |
| ETXS523 | GmsCmsUmGmCmCfUfGm | SEQ ID | GCUGCCUGCUCUUC | SEQ ID |
| | CfUfCfUfUmCmAmUmGm | NO: 1089 | AUGGGAA | NO: 409 |
| | GmGmAmAm | | | |
| ETXS525 | CmsAmsCmCmAmAfGfGm | SEQ ID | CACCAAGGGCCUCA | SEQ ID |
| | GfCfCfUfCmAmUmAmAm | NO: 1090 | UAAAAGA | NO: 410 |
| | AmAmGmAm | | | |
| ETXS527 | CmsCmsUmUmUmAfUfAm | SEQ ID | CCUUUAUAUCCAGA | SEQ ID |
| | UfCfCfAfGmAmAmGmCm | NO: 1091 | AGCAGUU | NO: 411 |
| | AmGmUmUm | | | |
| ETXS529 | CmsAmsAmCmUmGfCfAm | SEQ ID | CAACUGCAUCUACU | SEQ ID |
| | UfCfUfAfCmUmUmCmAm | NO: 1092 | UCAAAGG | NO: 412 |
| | AmAmGmGm | | | |
| ETXS531 | GmsAmsGmCmCmGfGfAm | SEQ ID | GAGCCGGAUCCAGC | SEQ ID |
| | UfCfCfAfGmCmGmUmCm | NO: 1093 | GUCUUAA | NO: 413 |
| | UmUmAmAm | | | |
| ETXS533 | AmsUmsCmCmAmGfCfGm | SEQ ID | AUCCAGCGUCUUAA | SEQ ID |
| | UfCfUfUfAmAmCmAmUm | NO: 1094 | CAUCCUC | NO: 414 |
| | CmCmUmCm | | | |
| ETXS535 | CmsAmsAmAmAmAfAfGm | SEQ ID | CAAAAAAGCAUGAC | SEQ ID |
| | CfAfUfGfAmCmAmAmAm | NO: 1095 | AAACAGA | NO: 415 |
| | CmAmGmAm | | | |
| ETXS537 | CmsAmsGmGmAmGfGfAm | SEQ ID | CAGGAGGAAUUUU | SEQ ID |
| | AfUfUfUfUmGmGmGmUm | NO: 1096 | GGGUACAC | NO: 416 |
| | AmCmAmCm | | | |
| ETXS539 | CmsAmsAmCmCmAfCfAm | SEQ ID | CAACCACAACUUCC | SEQ ID |
| | AfCfUfUfCmCmGmGmCm | NO: 1097 | GGCUGAA | NO: 417 |
| | UmGmAmAm | | | |
| ETXS541 | GmsGmsCmAmAmAfAfAm | SEQ ID | GGCAAAAAAGCAU | SEQ ID |
| | AfGfCfAfUmGmAmCmAm | NO: 1098 | GACAAACA | NO: 418 |
| | AmAmCmAm | | | |
| ETXS543 | GmsAmsAmUmAmAfAfUm | SEQ ID | GAAUAAAUUCCCAG | SEQ ID |
| | UfCfCfCfAmGmUmGmGm | NO: 1099 | UGGAAAU | NO: 419 |
| | AmAmAmUm | | | |
| ETXS545 | GmsGmsGmCmAmUfCfAm | SEQ ID | GGGCAUCAGCAUGC | SEQ ID |
| | GfCfAfUfGmCmUmAmAm | NO: 1100 | UAAUUGU | NO: 420 |
| | UmUmGmUm | | | |
| ETXS547 | AmsAmsAmAmAmGfCfAm | SEQ ID | AAAAAGCAUGACA | SEQ ID |
| | UfGfAfCfAmAmAmCmAm | NO: 1101 | AACAGAAC | NO: 421 |
| | GmAmAmCm | | | |
| ETXS549 | GmsAmsGmUmAmUfUfAm | SEQ ID | GAGUAUUACUUUG | SEQ ID |
| | CfUfUfUfGmCmUmGmAm | NO: 1102 | CUGAGGCC | NO: 422 |
| | GmGmCmCm | | | |
| ETXS551 | UmsGmsAmAmUmAmAfA | SEQ ID | UGAAUAAAUUCCCA | SEQ ID |
| | mUfUfCfCfCmAmGmUmG | NO: 1103 | GUGGAAA | NO: 403 |
| | mGmAmAmAm | | | |
| ETXS553 | CmsUmsGmCmAmUmCfU | SEQ ID | CUGCAUCUACUUCA | SEQ ID |
| | mAfCfUfUfCmAmAmAmG | NO: 1104 | AAGGAUC | NO: 404 |
| | mGmAmUmCm | | | |
| ETXS555 | AmsCmsUmGmCmAmUfC | SEQ ID | ACUGCAUCUACUUC | SEQ ID |
| | mUfAfCfUfUmCmAmAmA | NO: 1105 | AAAGGAU | NO: 405 |
| | mGmGmAmUm | | | |
| ETXS557 | GmsGmsGmAmGmAmGfA | SEQ ID | GGGAGAGACCCAUG | SEQ ID |
| | mCfCfCfAfUmGmAmAmC | NO: 1106 | AACAAGU | NO: 406 |
| | mAmAmGmUm | | | |
| ETXS559 | GmsGmsUmGmAmAmUfA | SEQ ID | GGUGAAUAAAUUC | SEQ ID |
| | mAfAfUfUfCmCmCmAmG | NO: 1107 | CCAGUGGA | NO: 407 |
| | mUmGmGmAm | | | |
| ETXS561 | UmsCmsAmGmGmAmGfG | SEQ ID | UCAGGAGGAAUUU | SEQ ID |
| | mAfAfUfUfUmUmGmGmG | NO: 1108 | UGGGUACA | NO: 408 |
| | mUmAmCmAm | | | |
| ETXS563 | GmsCmsUmGmCmCmUfG | SEQ ID | GCUGCCUGCUCUUC | SEQ ID |
| | mCfUfCfUfUmCmAmUmG | NO: 1109 | AUGGGAA | NO: 409 |
| | mGmGmAmAm | | | |
| ETXS565 | CmsAmsCmCmAmAmGfG | SEQ ID | CACCAAGGGCCUCA | SEQ ID |
| | mGfCfCfUfCmAmUmAmA | NO: 1110 | UAAAAGA | NO: 410 |
| | mAmAmGmAm | | | |
| ETXS567 | CmsCmsUmUmUmAmUfA | SEQ ID | CCUUUAUAUCCAGA | SEQ ID |
| | mUfCfCfAfGmAmAmGmC | NO: 1111 | AGCAGUU | NO: 411 |
| | mAmGmUmUm | | | |
| ETXS569 | CmsAmsAmCmUmGmCfA | SEQ ID | CAACUGCAUCUACU | SEQ ID |
| | mUfCfUfAfCmUmUmCmA | NO: 1112 | UCAAAGG | NO: 412 |
| | mAmAmGmGm | | | |
| ETXS571 | GmsAmsGmCmCmGmGfA | SEQ ID | GAGCCGGAUCCAGC | SEQ ID |
| | mUfCfCfAfGmCmGmUmC | NO: 1113 | GUCUUAA | NO: 413 |
| | mUmUmAmAm | | | |
| ETXS573 | AmsUmsCmCmAmGmCfG | SEQ ID | AUCCAGCGUCUUAA | SEQ ID |
| | mUfCfUfUfAmAmCmAmU | NO: 1114 | CAUCCUC | NO: 414 |
| | mCmCmUmCm | | | |
| ETXS575 | CmsAmsAmAmAmAmAfG | SEQ ID | CAAAAAAGCAUGAC | SEQ ID |
| | mCfAfUfGfAmCmAmAmA | NO: 1115 | AAACAGA | NO: 415 |
| | mCmAmGmAm | | | |
| ETXS577 | CmsAmsGmGmAmGmGfA | SEQ ID | CAGGAGGAAUUUU | SEQ ID |
| | mAfUfUfUfUmGmGmGmU | NO: 1116 | GGGUACAC | NO: 416 |
| | mAmCmAmCm | | | |
| ETXS579 | CmsAmsAmCmCmAmCfA | SEQ ID | CAACCACAACUUCC | SEQ ID |
| | mAfCfUfUfCmCmGmGmC | NO: 1117 | GGCUGAA | NO: 417 |
| | mUmGmAmAm | | | |
| ETXS581 | GmsGmsCmAmAmAmAfA | SEQ ID | GGCAAAAAAGCAU | SEQ ID |
| | mAfGfCfAfUmGmAmCmA | NO: 1118 | GACAAACA | NO: 418 |
| | mAmAmCmAm | | | |
| ETXS583 | GmsAmsAmUmAmAmAfU | SEQ ID | GAAUAAAUUCCCAG | SEQ ID |
| | mUfCfCfCfAmGmUmGmG | NO: 1119 | UGGAAAU | NO: 419 |
| | mAmAmAmUm | | | |
| ETXS585 | GmsGmsGmCmAmUmCfA | SEQ ID | GGGCAUCAGCAUGC | SEQ ID |
| | mGfCfAfUfGmCmUmAmA | NO: 1120 | UAAUUGU | NO: 420 |
| | mUmUmGmUm | | | |
| ETXS587 | AmsAmsAmAmAmGmCfA | SEQ ID | AAAAAGCAUGACA | SEQ ID |
| | mUfGfAfCfAmAmAmCmA | NO: 1121 | AACAGAAC | NO: 421 |
| | mGmAmAmCm | | | |
| ETXS589 | GmsAmsGmUmAmUmUfA | SEQ ID | GAGUAUUACUUUG | SEQ ID |
| | mCfUfUfUfGmCmUmGmA | NO: 1122 | CUGAGGCC | NO: 422 |
| | mGmGmCmCm | | | |
| ETXS591 | UmsGmsAmAmUmAmAfA | SEQ ID | UGAAUAAAUUCCCA | SEQ ID |
| | mUfUfCfCfCmAmGmUmG | NO: 1123 | GUGGAAA | NO: 403 |
| | mGmAmAmAm | | | |
| ETXS593 | CmsUmsGmCmAmUmCfU | SEQ ID | CUGCAUCUACUUCA | SEQ ID |
| | mAfCfUfUfCmAmAmAmG | NO: 1124 | AAGGAUC | NO: 404 |
| | mGmAmUmCm | | | |
| ETXS595 | AmsCmsUmGmCmAmUfC | SEQ ID | ACUGCAUCUACUUC | SEQ ID |
| | mUfAfCfUfUmCmAmAmA | NO: 1125 | AAAGGAU | NO: 405 |
| | mGmGmAmUm | | | |
| ETXS597 | GmsGmsGmAmGmAmGfA | SEQ ID | GGGAGAGACCCAUG | SEQ ID |
| | mCfCfCfAfUmGmAmAmC | NO: 1126 | AACAAGU | NO: 406 |
| | mAmAmGmUm | | | |
| ETXS599 | GmsGmsUmGmAmAmUfA | SEQ ID | GGUGAAUAAAUUC | SEQ ID |
| | mAfAfUfUfCmCmCmAmG | NO: 1127 | CCAGUGGA | NO: 407 |
| | mUmGmGmAm | | | |
| ETXS601 | UmsCmsAmGmGmAmGfG | SEQ ID | UCAGGAGGAAUUU | SEQ ID |
| | mAfAfUfUfUmUmGmGmG | NO: 1128 | UGGGUACA | NO: 408 |
| | mUmAmCmAm | | | |
| ETXS603 | GmsCmsUmGmCmCmUfG | SEQ ID | GCUGCCUGCUCUUC | SEQ ID |
| | mCfUfCfUfUmCmAmUmG | NO: 1129 | AUGGGAA | NO: 409 |
| | mGmGmAmAm | | | |
| ETXS605 | CmsAmsCmCmAmAmGfG | SEQ ID | CACCAAGGGCCUCA | SEQ ID |
| | mGfCfCfUfCmAmUmAmA | NO: 1130 | UAAAAGA | NO: 410 |
| | mAmAmGmAm | | | |
| ETXS607 | CmsCmsUmUmUmAmUfA | SEQ ID | CCUUUAUAUCCAGA | SEQ ID |
| | mUfCfCfAfGmAmAmGmC | NO: 1131 | AGCAGUU | NO: 411 |
| | mAmGmUmUm | | | |
| ETXS609 | CmsAmsAmCmUmGmCfA | SEQ ID | CAACUGCAUCUACU | SEQ ID |
| | mUfCfUfAfCmUmUmCmA | NO: 1132 | UCAAAGG | NO: 412 |
| | mAmAmGmGm | | | |
| ETXS611 | GmsAmsGmCmCmGmGfA | SEQ ID | GAGCCGGAUCCAGC | SEQ ID |
| | mUfCfCfAfGmCmGmUmC | NO: 1133 | GUCUUAA | NO: 413 |
| | mUmUmAmAm | | | |
| ETXS613 | AmsUmsCmCmAmGmCfG | SEQ ID | AUCCAGCGUCUUAA | SEQ ID |
| | mUfCfUfUfAmAmCmAmU | NO: 1134 | CAUCCUC | NO: 414 |
| | mCmCmUmCm | | | |
| ETXS615 | CmsAmsAmAmAmAmAfG | SEQ ID | CAAAAAAGCAUGAC | SEQ ID |
| | mCfAfUfGfAmCmAmAmA | NO: 1135 | AAACAGA | NO: 415 |
| | mCmAmGmAm | | | |
| ETXS617 | CmsAmsGmGmAmGmGfA | SEQ ID | CAGGAGGAAUUUU | SEQ ID |
| | mAfUfUfUfUmGmGmGmU | NO: 1136 | GGGUACAC | NO: 416 |
| | mAmCmAmCm | | | |
| ETXS619 | CmsAmsAmCmCmAmCfA | SEQ ID | CAACCACAACUUCC | SEQ ID |
| | mAfCfUfUfCmCmGmGmC | NO: 1137 | GGCUGAA | NO: 417 |
| | mUmGmAmAm | | | |
| ETXS621 | GmsGmsCmAmAmAmAfA | SEQ ID | GGCAAAAAAGCAU | SEQ ID |
| | mAfGfCfAfUmGmAmCmA | NO: 1138 | GACAAACA | NO: 418 |
| | mAmAmCmAm | | | |
| ETXS623 | GmsAmsAmUmAmAmAfU | SEQ ID | GAAUAAAUUCCCAG | SEQ ID |
| | mUfCfCfCfAmGmUmGmG | NO: 1139 | UGGAAAU | NO: 419 |
| | mAmAmAmUm | | | |
| ETXS625 | GmsGmsGmCmAmUmCfA | SEQ ID | GGGCAUCAGCAUGC | SEQ ID |
| | mGfCfAfUfGmCmUmAmA | NO: 1140 | UAAUUGU | NO: 420 |
| | mUmUmGmUm | | | |
| ETXS627 | AmsAmsAmAmAmGmCfA | SEQ ID | AAAAAGCAUGACA | SEQ ID |
| | mUfGfAfCfAmAmAmCmA | NO: 1141 | AACAGAAC | NO: 421 |
| | mGmAmAmCm | | | |
| ETXS629 | GmsAmsGmUmAmUmUfA | SEQ ID | GAGUAUUACUUUG | SEQ ID |
| | mCfUfUfUfGmCmUmGmA | NO: 1142 | CUGAGGCC | NO: 422 |
| | mGmGmCmCm | | | |
| ETXS631 | UmsGmsCmCmUmUmCfA | SEQ ID | UGCCUUCAUCCACA | SEQ ID |
| | mUfCfCfAmCmAmAmGmG | NO: 1143 | AGGAUUU | NO: 503 |
| | mAmUmUmUm | | | |
| ETXS633 | UmsGmsCmGmAmAmAfG | SEQ ID | UGCGAAAGAUCUCC | SEQ ID |
| | mAfUfCfUmCmCmAmUmG | NO: 1144 | AUGAGGC | NO: 504 |
| | mAmGmGmCm | | | |
| ETXS635 | GmsCmsUmGmGmUmGfG | SEQ ID | GCUGGUGGUCCUCA | SEQ ID |
| | mUfCfCfUmCmAmUmGmG | NO: 1145 | UGGAGAA | NO: 505 |
| | mAmGmAmAm | | | |
| ETXS637 | GmsCmsGmAmAmAmGfA | SEQ ID | GCGAAAGAUCUCCA | SEQ ID |
| | mUfCfUfCmCmAmUmGmA | NO: 1146 | UGAGGCA | NO: 506 |
| | mGmGmCmAm | | | |
| ETXS639 | GmsUmsAmUmGmAmGfA | SEQ ID | GUAUGAGAUGCAU | SEQ ID |
| | mUfGfCfAmUmGmAmGmC | NO: 1147 | GAGCUGCU | NO: 507 |
| | mUmGmCmUm | | | |
| ETXS641 | GmsUmsUmUmGmAmUfG | SEQ ID | GUUUGAUGAGAUU | SEQ ID |
| | mAfGfAfUmUmAmAmUm | NO: 1148 | AAUCCUGA | NO: 508 |
| | CmCmUmGmAm | | | |
| ETXS643 | UmsGmsAmGmAmUmUfA | SEQ ID | UGAGAUUAAUCCU | SEQ ID |
| | mAfUfCfCmUmGmAmAmA | NO: 1149 | GAAACCAA | NO: 509 |
| | mCmCmAmAm | | | |
| ETXS645 | UmsGmsAmUmGmAmGfA | SEQ ID | UGAUGAGAUUAAU | SEQ ID |
| | mUfUfAfAmUmCmCmUmG | NO: 1150 | CCUGAAAC | NO: 510 |
| | mAmAmAmCm | | | |
| ETXS647 | AmsUmsGmAmGmAmUfU | SEQ ID | AUGAGAUUAAUCC | SEQ ID |
| | mAfAfUfCmCmUmGmAmA | NO: 1151 | UGAAACCA | NO: 511 |
| | mAmCmCmAm | | | |
| ETXS649 | GmsAmsUmGmAmGmAfU | SEQ ID | GAUGAGAUUAAUC | SEQ ID |
| | mUfAfAfUmCmCmUmGmA | NO: 1152 | CUGAAACC | NO: 512 |
| | mAmAmCmCm | | | |
| ETXS651 | CmsUmsGmUmUmUmGfA | SEQ ID | CUGUUUGAUGAGA | SEQ ID |
| | mUfGfAfGmAmUmUmAm | NO: 1153 | UUAAUCCU | NO: 513 |
| | AmUmCmCmUm | | | |
| ETXS653 | UmsUmsUmUmGmCmCfU | SEQ ID | UUUUGCCUUCAUCC | SEQ ID |
| | mUfCfAfUmCmCmAmCmA | NO: 1154 | ACAAGGA | NO: 514 |
| | mAmGmGmAm | | | |
| ETXS655 | CmsGmsAmAmAmGmAfU | SEQ ID | CGAAAGAUCUCCAU | SEQ ID |
| | mCfUfCfCmAmUmGmAmG | NO: 1155 | GAGGCAC | NO: 515 |
| | mGmCmAmCm | | | |
| ETXS657 | UmsGmsCmUmGmGmUfG | SEQ ID | UGCUGGUGGUCCUC | SEQ ID |
| | mGfUfCfCmUmCmAmUmG | NO: 1156 | AUGGAGA | NO: 516 |
| | mGmAmGmAm | | | |
| ETXS659 | CmsCmsUmUmCmAmUfC | SEQ ID | CCUUCAUCCACAAG | SEQ ID |
| | mCfAfCfAmAmGmGmAmU | NO: 1157 | GAUUUUG | NO: 517 |
| | mUmUmUmGm | | | |
| ETXS661 | GmsAmsAmAmGmAmUfC | SEQ ID | GAAAGAUCUCCAUG | SEQ ID |
| | mUfCfCfAmUmGmAmGmG | NO: 1158 | AGGCACG | NO: 518 |
| | mCmAmCmGm | | | |
| ETXS663 | UmsUmsCmAmUmCmCfA | SEQ ID | UUCAUCCACAAGGA | SEQ ID |
| | mCfAfAfGmGmAmUmUm | NO: 1159 | UUUUGAU | NO: 519 |
| | UmUmGmAmUm | | | |
| ETXS665 | UmsUmsUmGmAmUmGfA | SEQ ID | UUUGAUGAGAUUA | SEQ ID |
| | mGfAfUfUmAmAmUmCmC | NO: 1160 | AUCCUGAA | NO: 520 |
| | mUmGmAmAm | | | |
| ETXS667 | UmsUmsGmCmCmUmUfC | SEQ ID | UUGCCUUCAUCCAC | SEQ ID |
| | mAfUfCfCmAmCmAmAmG | NO: 1161 | AAGGAUU | NO: 521 |
| | mGmAmUmUm | | | |
| ETXS669 | GmsAmsUmCmUmCmCfA | SEQ ID | GAUCUCCAUGAGGC | SEQ ID |
| | mUfGfAfGmGmCmAmCmG | NO: 1162 | ACGAUGG | NO: 522 |
| | mAmUmGmGm | | | |
| ETXS671 | CmsAmsUmGmCmUmGfG | SEQ ID | CAUGCUGGUGGUCC | SEQ ID |
| | mUfGfGfUmCmCmUmCmA | NO: 1163 | UCAUGGA | NO: 523 |
| | mUmGmGmAm | | | |
| ETXS673 | UmsUmsUmGmCmCmUfU | SEQ ID | UUUGCCUUCAUCCA | SEQ ID |
| | mCfAfUfCmCmAmCmAmA | NO: 1164 | CAAGGAU | NO: 524 |
| | mGmGmAmUm | | | |
| ETXS675 | GmsCmsCmUmUmCmAfU | SEQ ID | GCCUUCAUCCACAA | SEQ ID |
| | mCfCfAfCmAmAmGmGmA | NO: 1165 | GGAUUUU | NO: 525 |
| | mUmUmUmUm | | | |
| ETXS677 | UmsAmsCmCmAmAmGfG | SEQ ID | UACCAAGGAAAUGC | SEQ ID |
| | mAfAfAfUmGmCmCmAmC | NO: 1166 | CACCAUG | NO: 526 |
| | mCmAmUmGm | | | |
| ETXS679 | UmsUmsGmAmUmGmAfG | SEQ ID | UUGAUGAGAUUAA | SEQ ID |
| | mAfUfUfAmAmUmCmCmU | NO: 1167 | UCCUGAAA | NO: 527 |
| | mGmAmAmAm | | | |
| ETXS681 | CmsUmsUmCmAmUmCfC | SEQ ID | CUUCAUCCACAAGG | SEQ ID |
| | mAfCfAfAmGmGmAmUm | NO: 1168 | AUUUUGA | NO: 528 |
| | UmUmUmGmAm | | | |
| ETXS683 | AmsAmsAmGmAmUmCfU | SEQ ID | AAAGAUCUCCAUGA | SEQ ID |
| | mCfCfAfUmGmAmGmGmC | NO: 1169 | GGCACGA | NO: 529 |
| | mAmCmGmAm | | | |
| ETXS685 | UmsGmsUmUmUmGmAfU | SEQ ID | UGUUUGAUGAGAU | SEQ ID |
| | mGfAfGfAmUmUmAmAm | NO: 1170 | UAAUCCUG | NO: 530 |
| | UmCmCmUmGm | | | |
| ETXS687 | AmsUmsGmCmUmGmGfU | SEQ ID | AUGCUGGUGGUCCU | SEQ ID |
| | mGfGfUfCmCmUmCmAmU | NO: 1171 | CAUGGAG | NO: 531 |
| | mGmGmAmGm | | | |
| ETXS689 | GmsUmsUmUmUmGmCfC | SEQ ID | GUUUUGCCUUCAUC | SEQ ID |
| | mUfUfCfAmUmCmCmAmC | NO: 1172 | CACAAGG | NO: 532 |
| | mAmAmGmGm | | | |
| ETXS691 | CmsCmsAmCmCmAmUfG | SEQ ID | CCACCAUGCUGGUG | SEQ ID |
| | mCfUfGfGmUmGmGmUmC | NO: 1173 | GUCCUCA | NO: 533 |
| | mCmUmCmAm | | | |
| ETXS693 | CmsAmsCmCmAmUmGfC | SEQ ID | CACCAUGCUGGUGG | SEQ ID |
| | mUfGfGfUmGmGmUmCmC | NO: 1174 | UCCUCAU | NO: 534 |
| | mUmCmAmUm | | | |
| ETXS695 | AmsAmsGmAmUmCmUfC | SEQ ID | AAGAUCUCCAUGAG | SEQ ID |
| | mCfAfUfGmAmGmGmCmA | NO: 1175 | GCACGAU | NO: 535 |
| | mCmGmAmUm | | | |
| ETXS697 | AmsGmsUmUmUmUmGfC | SEQ ID | AGUUUUGCCUUCAU | SEQ ID |
| | mCfUfUfCmAmUmCmCmA | NO: 1176 | CCACAAG | NO: 536 |
| | mCmAmAmGm | | | |
| ETXS699 | AmsCmsCmAmUmGmCfU | SEQ ID | ACCAUGCUGGUGGU | SEQ ID |
| | mGfGfUfGmGmUmCmCmU | NO: 1177 | CCUCAUG | NO: 537 |
| | mCmAmUmGm | | | |
| ETXS701 | AmsGmsAmUmCmUmCfC | SEQ ID | AGAUCUCCAUGAGG | SEQ ID |
| | mAfUfGfAmGmGmCmAmC | NO: 1178 | CACGAUG | NO: 538 |
| | mGmAmUmGm | | | |
| ETXS703 | CmsCmsAmUmGmCmUfG | SEQ ID | CCAUGCUGGUGGUC | SEQ ID |
| | mGfUfGfGmUmCmCmUmC | NO: 1179 | CUCAUGG | NO: 539 |
| | mAmUmGmGm | | | |
| ETXS705 | UmsGmsCmCmUmCmCfA | SEQ ID | UGCCUCCACCUUUG | SEQ ID |
| | mCfCfUfUmUmGmAmCmA | NO: 1180 | ACAAGAA | NO: 540 |
| | mAmGmAmAm | | | |
| ETXS707 | CmsCmsUmCmCmAmCfCm | SEQ ID | CCUCCACCUUUGAC | SEQ ID |
| | UfUfUfGmAmCmAmAmG | NO: 1181 | AAGAAUU | NO: 541 |
| | mAmAmUmUm | | | |
| ETXS709 | CmsAmsUmUmAmAmGfG | SEQ ID | CAUUAAGGUGCCCA | SEQ ID |
| | mUfGfCfCmCmAmUmGmA | NO: 1182 | UGAUGUA | NO: 542 |
| | mUmGmUmAm | | | |
| ETXS711 | CmsUmsGmCmCmCmUfA | SEQ ID | CUGCCCUACCAAGG | SEQ ID |
| | mCfCfAfAmGmGmAmAmA | NO: 1183 | AAAUGCC | NO: 543 |
| | mUmGmCmCm | | | |
| ETXS713 | CmsAmsAmAmCmUmGfC | SEQ ID | CAAACUGCCCUACC | SEQ ID |
| | mCfCfUfAmCmCmAmAmG | NO: 1184 | AAGGAAA | NO: 544 |
| | mGmAmAmAm | | | |
| ETXS715 | UmsCmsAmAmAmCmUfG | SEQ ID | UCAAACUGCCCUAC | SEQ ID |
| | mCfCfCfUmAmCmCmAmA | NO: 1185 | CAAGGAA | NO: 545 |
| | mGmGmAmAm | | | |
| ETXS717 | CmsUmsCmCmAmCmCfUm | SEQ ID | CUCCACCUUUGACA | SEQ ID |
| | UfUfGfAmCmAmAmGmA | NO: 1186 | AGAAUUU | NO: 546 |
| | mAmUmUmUm | | | |
| ETXS719 | CmsGmsAmCmAmCmUfU | SEQ ID | CGACACUUUCCACC | SEQ ID |
| | mUfCfCfAmCmCmUmGmG | NO: 1187 | UGGACAA | NO: 547 |
| | mAmCmAmAm | | | |
| ETXS721 | AmsCmsUmUmUmCmCfA | SEQ ID | ACUUUCCACCUGGA | SEQ ID |
| | mCfCfUfGmGmAmCmAmA | NO: 1188 | CAAGUAC | NO: 548 |
| | mGmUmAmCm | | | |
| ETXS723 | AmsAmsAmCmUmGmCfC | SEQ ID | AAACUGCCCUACCA | SEQ ID |
| | mCfUfAfCmCmAmAmGmG | NO: 1189 | AGGAAAU | NO: 549 |
| | mAmAmAmUm | | | |
| ETXS725 | AmsCmsUmGmCmCmCfU | SEQ ID | ACUGCCCUACCAAG | SEQ ID |
| | mAfCfCfAmAmGmGmAmA | NO: 1190 | GAAAUGC | NO: 550 |
| | mAmUmGmCm | | | |
| ETXS727 | CmsAmsCmUmUmUmCfC | SEQ ID | CACUUUCCACCUGG | SEQ ID |
| | mAfCfCfUmGmGmAmCmA | NO: 1191 | ACAAGUA | NO: 551 |
| | mAmGmUmAm | | | |
| ETXS729 | CmsCmsAmUmUmAmAfG | SEQ ID | CCAUUAAGGUGCCC | SEQ ID |
| | mGfUfGfCmCmCmAmUmG | NO: 1192 | AUGAUGU | NO: 552 |
| | mAmUmGmUm | | | |
| ETXS731 | UmsUmsGmCmCmUmCfC | SEQ ID | UUGCCUCCACCUUU | SEQ ID |
| | mAfCfCfUmUmUmGmAmC | NO: 1193 | GACAAGA | NO: 553 |
| | mAmAmGmAm | | | |
| ETXS733 | UmsUmsUmCmCmAmCfC | SEQ ID | UUUCCACCUGGACA | SEQ ID |
| | mUfGfGfAmCmAmAmGm | NO: 1194 | AGUACAA | NO: 554 |
| | UmAmCmAmAm | | | |
| ETXS735 | UmsCmsCmUmCmAmAfA | SEQ ID | UCCUCAAACUGCCC | SEQ ID |
| | mCfUfGfCmCmCmUmAmC | NO: 1195 | UACCAAG | NO: 555 |
| | mCmAmAmGm | | | |
| ETXS737 | AmsAmsCmUmGmCmCfC | SEQ ID | AACUGCCCUACCAA | SEQ ID |
| | mUfAfCfCmAmAmGmGmA | NO: 1196 | GGAAAUG | NO: 556 |
| | mAmAmUmGm | | | |
| ETXS739 | CmsCmsUmCmAmAmAfC | SEQ ID | CCUCAAACUGCCCU | SEQ ID |
| | mUfGfCfCmCmUmAmCmC | NO: 1197 | ACCAAGG | NO: 557 |
| | mAmAmGmGm | | | |
| ETXS741 | GmsUmsCmCmUmCmAfA | SEQ ID | GUCCUCAAACUGCC | SEQ ID |
| | mAfCfUfGmCmCmCmUmA | NO: 1198 | CUACCAA | NO: 558 |
| | mCmCmAmAm | | | |
| ETXS743 | GmsAmsCmAmCmUmUfU | SEQ ID | GACACUUUCCACCU | SEQ ID |
| | mCfCfAfCmCmUmGmGmA | NO: 1199 | GGACAAG | NO: 559 |
| | mCmAmAmGm | | | |
| ETXS745 | UmsUmsUmGmCmCmUfC | SEQ ID | UUUGCCUCCACCUU | SEQ ID |
| | mCfAfCfCmUmUmUmGmA | NO: 1200 | UGACAAG | NO: 560 |
| | mCmAmAmGm | | | |
| ETXS747 | AmsCmsAmCmUmUmUfC | SEQ ID | ACACUUUCCACCUG | SEQ ID |
| | mCfAfCfCmUmGmGmAmC | NO: 1201 | GACAAGU | NO: 561 |
| | mAmAmGmUm | | | |
| ETXS749 | UmsUmsCmCmAmCmCfU | SEQ ID | UUCCACCUGGACAA | SEQ ID |
| | mGfGfAfCmAmAmGmUm | NO: 1202 | GUACAAG | NO: 562 |
| | AmCmAmAmGm | | | |
| ETXS751 | CmsUmsUmUmCmCmAfC | SEQ ID | CUUUCCACCUGGAC | SEQ ID |
| | mCfUfGfGmAmCmAmAmG | NO: 1203 | AAGUACA | NO: 563 |
| | mUmAmCmAm | | | |
| ETXS753 | CmsUmsCmAmAmAmCfU | SEQ ID | CUCAAACUGCCCUA | SEQ ID |
| | mGfCfCfCmUmAmCmCmA | NO: 1204 | CCAAGGA | NO: 564 |
| | mAmGmGmAm | | | |
| ETXS755 | UmsUmsAmCmAmUmCfU | SEQ ID | UUACAUCUUGUUCA | SEQ ID |
| | mUfGfUfUmCmAmAmAm | NO: 1205 | AAGGGAA | NO: 565 |
| | GmGmGmAmAm | | | |
| ETXS757 | UmsGmsCmCmAmCmCfA | SEQ ID | UGCCACCAUGCUGG | SEQ ID |
| | mUfGfCfUmGmGmUmGm | NO: 1206 | UGGUCCU | NO: 566 |
| | GmUmCmCmUm | | | |
| ETXS759 | UmsAmsUmCmCmAmAfG | SEQ ID | UAUCCAAGAGGUA | SEQ ID |
| | mAfGfGfUmAmUmUmUm | NO: 1207 | UUUUGAUA | NO: 567 |
| | UmGmAmUmAm | | | |
| ETXS761 | AmsAmsAmUmGmCmCfA | SEQ ID | AAAUGCCACCAUGC | SEQ ID |
| | mCfCfAfUmGmCmUmGmG | NO: 1208 | UGGUGGU | NO: 568 |
| | mUmGmGmUm | | | |
| ETXS763 | AmsUmsUmAmCmAmUfC | SEQ ID | AUUACAUCUUGUUC | SEQ ID |
| | mUfUfGfUmUmCmAmAm | NO: 1209 | AAAGGGA | NO: 569 |
| | AmGmGmGmAm | | | |
| ETXS765 | CmsUmsCmCmAmUmGfA | SEQ ID | CUCCAUGAGGCACG | SEQ ID |
| | mGfGfCfAmCmGmAmUmG | NO: 1210 | AUGGCAA | NO: 570 |
| | mGmCmAmAm | | | |
| ETXS767 | UmsCmsCmAmUmGmCfC | SEQ ID | UCCAUGCCUCCUGU | SEQ ID |
| | mUfCfCfUmGmUmCmAmU | NO: 1211 | CAUCAAA | NO: 571 |
| | mCmAmAmAm | | | |
| ETXS769 | AmsUmsUmCmCmAmUfG | SEQ ID | AUUCCAUGCCUCCU | SEQ ID |
| | mCfCfUfCmCmUmGmUmC | NO: 1212 | GUCAUCA | NO: 572 |
| | mAmUmCmAm | | | |
| ETXS771 | CmsAmsAmGmGmAmAfA | SEQ ID | CAAGGAAAUGCCAC | SEQ ID |
| | mUfGfCfCmAmCmCmAmU | NO: 1213 | CAUGCUG | NO: 573 |
| | mGmCmUmGm | | | |
| ETXS773 | UmsGmsGmUmGmGmUfC | SEQ ID | UGGUGGUCCUCAUG | SEQ ID |
| | mCfUfCfAmUmGmGmAmG | NO: 1214 | GAGAAAA | NO: 574 |
| | mAmAmAmAm | | | |
| ETXS775 | AmsUmsCmUmUmGmUfU | SEQ ID | AUCUUGUUCAAAG | SEQ ID |
| | mCfAfAfAmGmGmGmAm | NO: 1215 | GGAAAUGG | NO: 575 |
| | AmAmUmGmGm | | | |
| ETXS777 | AmsAmsGmGmAmAmAfU | SEQ ID | AAGGAAAUGCCACC | SEQ ID |
| | mGfCfCfAmCmCmAmUmG | NO: 1216 | AUGCUGG | NO: 576 |
| | mCmUmGmGm | | | |
| ETXS779 | GmsCmsCmUmCmCmAfCm | SEQ ID | GCCUCCACCUUUGA | SEQ ID |
| | CfUfUfUmGmAmCmAmAm | NO: 1217 | CAAGAAU | NO: 577 |
| | GmAmAmUm | | | |
| ETXS781 | GmsAmsGmUmUmUmUfG | SEQ ID | GAGUUUUGCCUUCA | SEQ ID |
| | mCfCfUfUmCmAmUmCmC | NO: 1218 | UCCACAA | NO: 578 |
| | mAmCmAmAm | | | |
| ETXS783 | GmsCmsUmGmCmGmAfA | SEQ ID | GCUGCGAAAGAUCU | SEQ ID |
| | mAfGfAfUmCmUmCmCmA | NO: 1219 | CCAUGAG | NO: 579 |
| | mUmGmAmGm | | | |
| ETXS785 | AmsGmsUmUmUmGmCfC | SEQ ID | AGUUUGCCUCCACC | SEQ ID |
| | mUfCfCfAmCmCmUmUmU | NO: 1220 | UUUGACA | NO: 580 |
| | mGmAmCmAm | | | |
| ETXS787 | GmsUmsUmUmGmCmCfU | SEQ ID | GUUUGCCUCCACCU | SEQ ID |
| | mCfCfAfCmCmUmUmUmG | NO: 1221 | UUGACAA | NO: 581 |
| | mAmCmAmAm | | | |
| ETXS789 | CmsAmsUmCmUmUmGfU | SEQ ID | CAUCUUGUUCAAAG | SEQ ID |
| | mUfCfAfAmAmGmGmGm | NO: 1222 | GGAAAUG | NO: 582 |
| | AmAmAmUmGm | | | |
| ETXS791 | GmsGmsAmGmUmUmUfU | SEQ ID | GGAGUUUUGCCUUC | SEQ ID |
| | mGfCfCfUmUmCmAmUmC | NO: 1223 | AUCCACA | NO: 583 |
| | mCmAmCmAm | | | |
| ETXS793 | CmsUmsGmCmGmAmAfA | SEQ ID | CUGCGAAAGAUCUC | SEQ ID |
| | mGfAfUfCmUmCmCmAmU | NO: 1224 | CAUGAGG | NO: 584 |
| | mGmAmGmGm | | | |
| ETXS795 | CmsCmsAmUmGmCmCfU | SEQ ID | CCAUGCCUCCUGUC | SEQ ID |
| | mCfCfUfGmUmCmAmUmC | NO: 1225 | AUCAAAG | NO: 585 |
| | mAmAmAmGm | | | |
| ETXS797 | UmsGmsUmUmUmCmUfG | SEQ ID | UGUUUCUGGGCAG | SEQ ID |
| | mGfGfCfAmGmGmGmUm | NO: 1226 | GGUGGUGA | NO: 586 |
| | GmGmUmGmAm | | | |
| ETXS799 | GmsCmsCmUmGmCmUfG | SEQ ID | GCCUGCUGCGAAAG | SEQ ID |
| | mCfGfAfAmAmGmAmUmC | NO: 1227 | AUCUCCA | NO: 587 |
| | mUmCmCmAm | | | |
| ETXS801 | AmsAmsCmUmGmUmUfU | SEQ ID | AACUGUUUGAUGA | SEQ ID |
| | mGfAfUfGmAmGmAmUm | NO: 1228 | GAUUAAUC | NO: 588 |
| | UmAmAmUmCm | | | |
| ETXS803 | AmsUmsGmCmCmUmCfC | SEQ ID | AUGCCUCCUGUCAU | SEQ ID |
| | mUfGfUfCmAmUmCmAmA | NO: 1229 | CAAAGUG | NO: 589 |
| | mAmGmUmGm | | | |
| ETXS805 | AmsGmsUmAmUmGmAfG | SEQ ID | AGUAUGAGAUGCA | SEQ ID |
| | mAfUfGfCmAmUmGmAm | NO: 1230 | UGAGCUGC | NO: 590 |
| | GmCmUmGmCm | | | |
| ETXS807 | AmsAmsGmUmAmUmGfA | SEQ ID | AAGUAUGAGAUGC | SEQ ID |
| | mGfAfUfGmCmAmUmGm | NO: 1231 | AUGAGCUG | NO: 591 |
| | AmGmCmUmGm | | | |
| ETXS809 | UmsGmsUmCmCmUmCfA | SEQ ID | UGUCCUCAAACUGC | SEQ ID |
| | mAfAfCfUmGmCmCmCmU | NO: 1232 | CCUACCA | NO: 592 |
| | mAmCmCmAm | | | |
| ETXS811 | AmsCmsUmGmUmUmUfG | SEQ ID | ACUGUUUGAUGAG | SEQ ID |
| | mAfUfGfAmGmAmUmUm | NO: 1233 | AUUAAUCC | NO: 593 |
| | AmAmUmCmCm | | | |
| ETXS813 | GmsAmsCmCmAmUmUfA | SEQ ID | GACCAUUAAGGUGC | SEQ ID |
| | mAfGfGfUmGmCmCmCmA | NO: 1234 | CCAUGAU | NO: 594 |
| | mUmGmAmUm | | | |
| ETXS815 | AmsCmsCmAmUmUmAfA | SEQ ID | ACCAUUAAGGUGCC | SEQ ID |
| | mGfGfUfGmCmCmCmAmU | NO: 1235 | CAUGAUG | NO: 595 |
| | mGmAmUmGm | | | |
| ETXS817 | GmsUmsUmUmCmUmGfG | SEQ ID | GUUUCUGGGCAGG | SEQ ID |
| | mGfCfAfGmGmGmUmGm | NO: 1236 | GUGGUGAA | NO: 596 |
| | GmUmGmAmAm | | | |
| ETXS819 | UmsUmsCmUmGmUmUfU | SEQ ID | UUCUGUUUCUGGGC | SEQ ID |
| | mCfUfGfGmGmCmAmGmG | NO: 1237 | AGGGUGG | NO: 597 |
| | mGmUmGmGm | | | |
| ETXS821 | UmsUmsUmUmCmUmUfU | SEQ ID | UUUUCUUUCCGAAG | SEQ ID |
| | mCfCfGfAmAmGmUmUmC | NO: 1238 | UUCAAGC | NO: 598 |
| | mAmAmGmCm | | | |
| ETXS823 | UmsUmsUmCmUmUmUfC | SEQ ID | UUUCUUUCCGAAGU | SEQ ID |
| | mCfGfAfAmGmUmUmCmA | NO: 1239 | UCAAGCU | NO: 599 |
| | mAmGmCmUm | | | |
| ETXS825 | UmsCmsUmGmUmUmUfC | SEQ ID | UCUGUUUCUGGGCA | SEQ ID |
| | mUfGfGfGmCmAmGmGm | NO: 1240 | GGGUGGU | NO: 600 |
| | GmUmGmGmUm | | | |
| ETXS827 | AmsUmsGmUmCmCmUfC | SEQ ID | AUGUCCUCAAACUG | SEQ ID |
| | mAfAfAfCmUmGmCmCmC | NO: 1241 | CCCUACC | NO: 601 |
| | mUmAmCmCm | | | |
| ETXS829 | UmsCmsGmAmCmAmCfU | SEQ ID | UCGACACUUUCCAC | SEQ ID |
| | mUfUfCfCmAmCmCmUmG | NO: 1242 | CUGGACA | NO: 602 |
| | mGmAmCmAm | | | |
| ETXS871 | UmsGmsCmCmUmUmCfAf | SEQ ID | UGCCUUCAUCCACA | SEQ ID |
| | UfCfCfAmCmAmAmGmGm | NO: 1243 | AGGAUUU | NO: 503 |
| | AmUfUmUm | | | |
| ETXS873 | UmsGmsCmGmAmAmAfGf | SEQ ID | UGCGAAAGAUCUCC | SEQ ID |
| | AfUfCfUmCmCmAmUmGm | NO: 1244 | AUGAGGC | NO: 504 |
| | AmGfGmCm | | | |
| ETXS875 | GmsCmsUmGmGmUmGfGf | SEQ ID | GCUGGUGGUCCUCA | SEQ ID |
| | UfCfCfUmCmAmUmGmGm | NO: 1245 | UGGAGAA | NO: 505 |
| | AmGfAmAm | | | |
| ETXS877 | GmsCmsGmAmAmAmGfAf | SEQ ID | GCGAAAGAUCUCCA | SEQ ID |
| | UfCfUfCmCmAmUmGmAm | NO: 1246 | UGAGGCA | NO: 506 |
| | GmGfCmAm | | | |
| ETXS879 | GmsUmsAmUmGmAmGfAf | SEQ ID | GUAUGAGAUGCAU | SEQ ID |
| | UfGfCfAmUmGmAmGmCm | NO: 1247 | GAGCUGCU | NO: 507 |
| | UmGfCmUm | | | |
| ETXS881 | GmsUmsUmUmGmAmUfGf | SEQ ID | GUUUGAUGAGAUU | SEQ ID |
| | AfGfAfUmUmAmAmUmC | NO: 1248 | AAUCCUGA | NO: 508 |
| | mCmUfGmAm | | | |
| ETXS883 | UmsGmsAmGmAmUmUfAf | SEQ ID | UGAGAUUAAUCCU | SEQ ID |
| | AfUfCfCmUmGmAmAmAm | NO: 1249 | GAAACCAA | NO: 509 |
| | CmCfAmAm | | | |
| ETXS885 | UmsGmsAmUmGmAmGfAf | SEQ ID | UGAUGAGAUUAAU | SEQ ID |
| | UfUfAfAmUmCmCmUmGm | NO: 1250 | CCUGAAAC | NO: 510 |
| | AmAfAmCm | | | |
| ETXS887 | AmsUmsGmAmGmAmUfUf | SEQ ID | AUGAGAUUAAUCC | SEQ ID |
| | AfAfUfCmCmUmGmAmAm | NO: 1251 | UGAAACCA | NO: 511 |
| | AmCfCmAm | | | |
| ETXS889 | GmsAmsUmGmAmGmAfUf | SEQ ID | GAUGAGAUUAAUC | SEQ ID |
| | UfAfAfUmCmCmUmGmAm | NO: 1252 | CUGAAACC | NO: 512 |
| | AmAfCmCm | | | |
| ETXS891 | CmsUmsGmUmUmUmGfAf | SEQ ID | CUGUUUGAUGAGA | SEQ ID |
| | UfGfAfGmAmUmUmAmA | NO: 1253 | UUAAUCCU | NO: 513 |
| | mUmCfCmUm | | | |
| ETXS893 | UmsUmsUmUmGmCmCfUf | SEQ ID | UUUUGCCUUCAUCC | SEQ ID |
| | UfCfAfUmCmCmAmCmAm | NO: 1254 | ACAAGGA | NO: 514 |
| | AmGfGmAm | | | |
| ETXS895 | CmsGmsAmAmAmGmAfUf | SEQ ID | CGAAAGAUCUCCAU | SEQ ID |
| | CfUfCfCmAmUmGmAmGm | NO: 1255 | GAGGCAC | NO: 515 |
| | GmCfAmCm | | | |
| ETXS897 | UmsGmsCmUmGmGmUfGf | SEQ ID | UGCUGGUGGUCCUC | SEQ ID |
| | GfUfCfCmUmCmAmUmGm | NO: 1256 | AUGGAGA | NO: 516 |
| | GmAfGmAm | | | |
| ETXS899 | CmsCmsUmUmCmAmUfCf | SEQ ID | CCUUCAUCCACAAG | SEQ ID |
| | CfAfCfAmAmGmGmAmUm | NO: 1257 | GAUUUUG | NO: 517 |
| | UmUfUmGm | | | |
| ETXS901 | GmsAmsAmAmGmAmUfCf | SEQ ID | GAAAGAUCUCCAUG | SEQ ID |
| | UfCfCfAmUmGmAmGmGm | NO: 1258 | AGGCACG | NO: 518 |
| | CmAfCmGm | | | |
| ETXS903 | UmsUmsCmAmUmCmCfAf | SEQ ID | UUCAUCCACAAGGA | SEQ ID |
| | CfAfAfGmGmAmUmUmU | NO: 1259 | UUUUGAU | NO: 519 |
| | mUmGfAmUm | | | |
| ETXS905 | UmsUmsUmGmAmUmGfAf | SEQ ID | UUUGAUGAGAUUA | SEQ ID |
| | GfAfUfUmAmAmUmCmCm | NO: 1260 | AUCCUGAA | NO: 520 |
| | UmGfAmAm | | | |
| ETXS907 | UmsUmsGmCmCmUmUfCf | SEQ ID | UUGCCUUCAUCCAC | SEQ ID |
| | AfUfCfCmAmCmAmAmGm | NO: 1261 | AAGGAUU | NO: 521 |
| | GmAfUmUm | | | |
| ETXS909 | GmsAmsUmCmUmCmCfAf | SEQ ID | GAUCUCCAUGAGGC | SEQ ID |
| | UfGfAfGmGmCmAmCmGm | NO: 1262 | ACGAUGG | NO: 522 |
| | AmUfGmGm | | | |
| ETXS911 | UmsGmsCmCmUmUfCfAm | SEQ ID | UGCCUUCAUCCACA | SEQ ID |
| | UfCfCfAfCmAmAmGmGm | NO: 1263 | AGGAUUU | NO: 503 |
| | AmUmUmUm | | | |
| ETXS913 | UmsGmsCmGmAmAfAfGm | SEQ ID | UGCGAAAGAUCUCC | SEQ ID |
| | AfUfCfUfCmCmAmUmGm | NO: 1264 | AUGAGGC | NO: 504 |
| | AmGmGmCm | | | |
| ETXS915 | GmsCmsUmGmGmUfGfGm | SEQ ID | GCUGGUGGUCCUCA | SEQ ID |
| | UfCfCfUfCmAmUmGmGm | NO: 1265 | UGGAGAA | NO: 505 |
| | AmGmAmAm | | | |
| ETXS917 | GmsCmsGmAmAmAfGfAm | SEQ ID | GCGAAAGAUCUCCA | SEQ ID |
| | UfCfUfCfCmAmUmGmAm | NO: 1266 | UGAGGCA | NO: 506 |
| | GmGmCmAm | | | |
| ETXS919 | GmsUmsAmUmGmAfGfAm | SEQ ID | GUAUGAGAUGCAU | SEQ ID |
| | UfGfCfAfUmGmAmGmCm | NO: 1267 | GAGCUGCU | NO: 507 |
| | UmGmCmUm | | | |
| ETXS921 | GmsUmsUmUmGmAfUfGm | SEQ ID | GUUUGAUGAGAUU | SEQ ID |
| | AfGfAfUfUmAmAmUmCm | NO: 1268 | AAUCCUGA | NO: 508 |
| | CmUmGmAm | | | |
| ETXS923 | UmsGmsAmGmAmUfUfAm | SEQ ID | UGAGAUUAAUCCU | SEQ ID |
| | AfUfCfCfUmGmAmAmAm | NO: 1269 | GAAACCAA | NO: 509 |
| | CmCmAmAm | | | |
| ETXS925 | UmsGmsAmUmGmAfGfAm | SEQ ID | UGAUGAGAUUAAU | SEQ ID |
| | UfUfAfAfUmCmCmUmGm | NO: 1270 | CCUGAAAC | NO: 510 |
| | AmAmAmCm | | | |
| ETXS927 | AmsUmsGmAmGmAfUfUm | SEQ ID | AUGAGAUUAAUCC | SEQ ID |
| | AfAfUfCfCmUmGmAmAm | NO: 1271 | UGAAACCA | NO: 511 |
| | AmCmCmAm | | | |
| ETXS929 | GmsAmsUmGmAmGfAfUm | SEQ ID | GAUGAGAUUAAUC | SEQ ID |
| | UfAfAfUfCmCmUmGmAm | NO: 1272 | CUGAAACC | NO: 512 |
| | AmAmCmCm | | | |
| ETXS931 | CmsUmsGmUmUmUfGfAm | SEQ ID | CUGUUUGAUGAGA | SEQ ID |
| | UfGfAfGfAmUmUmAmAm | NO: 1273 | UUAAUCCU | NO: 513 |
| | UmCmCmUm | | | |
| ETXS933 | UmsUmsUmUmGmCfCfUm | SEQ ID | UUUUGCCUUCAUCC | SEQ ID |
| | UfCfAfUfCmCmAmCmAm | NO: 1274 | ACAAGGA | NO: 514 |
| | AmGmGmAm | | | |
| ETXS935 | CmsGmsAmAmAmGfAfUm | SEQ ID | CGAAAGAUCUCCAU | SEQ ID |
| | CfUfCfCfAmUmGmAmGm | NO: 1275 | GAGGCAC | NO: 515 |
| | GmCmAmCm | | | |
| ETXS937 | UmsGmsCmUmGmGfUfGm | SEQ ID | UGCUGGUGGUCCUC | SEQ ID |
| | GfUfCfCfUmCmAmUmGm | NO: 1276 | AUGGAGA | NO: 516 |
| | GmAmGmAm | | | |
| ETXS939 | CmsCmsUmUmCmAfUfCm | SEQ ID | CCUUCAUCCACAAG | SEQ ID |
| | CfAfCfAfAmGmGmAmUm | NO: 1277 | GAUUUUG | NO: 517 |
| | UmUmUmGm | | | |
| ETXS941 | GmsAmsAmAmGmAfUfCm | SEQ ID | GAAAGAUCUCCAUG | SEQ ID |
| | UfCfCfAfUmGmAmGmGm | NO: 1278 | AGGCACG | NO: 518 |
| | CmAmCmGm | | | |
| ETXS943 | UmsUmsCmAmUmCfCfAm | SEQ ID | UUCAUCCACAAGGA | SEQ ID |
| | CfAfAfGfGmAmUmUmUm | NO: 1279 | UUUUGAU | NO: 519 |
| | UmGmAmUm | | | |
| ETXS945 | UmsUmsUmGmAmUfGfAm | SEQ ID | UUUGAUGAGAUUA | SEQ ID |
| | GfAfUfUfAmAmUmCmCm | NO: 1280 | AUCCUGAA | NO: 520 |
| | UmGmAmAm | | | |
| ETXS947 | UmsUmsGmCmCmUfUfCm | SEQ ID | UUGCCUUCAUCCAC | SEQ ID |
| | AfUfCfCfAmCmAmAmGm | NO: 1281 | AAGGAUU | NO: 521 |
| | GmAmUmUm | | | |
| ETXS949 | GmsAmsUmCmUmCfCfAm | SEQ ID | GAUCUCCAUGAGGC | SEQ ID |
| | UfGfAfGfGmCmAmCmGm | NO: 1282 | ACGAUGG | NO: 522 |
| | AmUmGmGm | | | |
| ETXS951 | UmsGmsCmCmUmUmCfA | SEQ ID | UGCCUUCAUCCACA | SEQ ID |
| | mUfCfCfAfCmAmAmGmG | NO: 1283 | AGGAUUU | NO: 503 |
| | mAmUmUmUm | | | |
| ETXS953 | UmsGmsCmGmAmAmAfG | SEQ ID | UGCGAAAGAUCUCC | SEQ ID |
| | mAfUfCfUfCmCmAmUmG | NO: 1284 | AUGAGGC | NO: 504 |
| | mAmGmGmCm | | | |
| ETXS955 | GmsCmsUmGmGmUmGfG | SEQ ID | GCUGGUGGUCCUCA | SEQ ID |
| | mUfCfCfUfCmAmUmGmG | NO: 1285 | UGGAGAA | NO: 505 |
| | mAmGmAmAm | | | |
| ETXS957 | GmsCmsGmAmAmAmGfA | SEQ ID | GCGAAAGAUCUCCA | SEQ ID |
| | mUfCfUfCfCmAmUmGmA | NO: 1286 | UGAGGCA | NO: 506 |
| | mGmGmCmAm | | | |
| ETXS959 | GmsUmsAmUmGmAmGfA | SEQ ID | GUAUGAGAUGCAU | SEQ ID |
| | mUfGfCfAfUmGmAmGmC | NO: 1287 | GAGCUGCU | NO: 507 |
| | mUmGmCmUm | | | |
| ETXS961 | GmsUmsUmUmGmAmUfG | SEQ ID | GUUUGAUGAGAUU | SEQ ID |
| | mAfGfAfUfUmAmAmUmC | NO: 1288 | AAUCCUGA | NO: 508 |
| | mCmUmGmAm | | | |
| ETXS963 | UmsGmsAmGmAmUmUfA | SEQ ID | UGAGAUUAAUCCU | SEQ ID |
| | mAfUfCfCfUmGmAmAmA | NO: 1289 | GAAACCAA | NO: 509 |
| | mCmCmAmAm | | | |
| ETXS965 | UmsGmsAmUmGmAmGfA | SEQ ID | UGAUGAGAUUAAU | SEQ ID |
| | mUfUfAfAfUmCmCmUmG | NO: 1290 | CCUGAAAC | NO: 510 |
| | mAmAmAmCm | | | |
| ETXS967 | AmsUmsGmAmGmAmUfU | SEQ ID | AUGAGAUUAAUCC | SEQ ID |
| | mAfAfUfCfCmUmGmAmA | NO: 1291 | UGAAACCA | NO: 511 |
| | mAmCmCmAm | | | |
| ETXS969 | GmsAmsUmGmAmGmAfU | SEQ ID | GAUGAGAUUAAUC | SEQ ID |
| | mUfAfAfUfCmCmUmGmA | NO: 1292 | CUGAAACC | NO: 512 |
| | mAmAmCmCm | | | |
| ETXS971 | CmsUmsGmUmUmUmGfA | SEQ ID | CUGUUUGAUGAGA | SEQ ID |
| | mUfGfAfGfAmUmUmAmA | NO: 1293 | UUAAUCCU | NO: 513 |
| | mUmCmCmUm | | | |
| ETXS973 | UmsUmsUmUmGmCmCfU | SEQ ID | UUUUGCCUUCAUCC | SEQ ID |
| | mUfCfAfUfCmCmAmCmA | NO: 1294 | ACAAGGA | NO: 514 |
| | mAmGmGmAm | | | |
| ETXS975 | CmsGmsAmAmAmGmAfU | SEQ ID | CGAAAGAUCUCCAU | SEQ ID |
| | mCfUfCfCfAmUmGmAmG | NO: 1295 | GAGGCAC | NO: 515 |
| | mGmCmAmCm | | | |
| ETXS977 | UmsGmsCmUmGmGmUfG | SEQ ID | UGCUGGUGGUCCUC | SEQ ID |
| | mGfUfCfCfUmCmAmUmG | NO: 1296 | AUGGAGA | NO: 516 |
| | mGmAmGmAm | | | |
| ETXS979 | CmsCmsUmUmCmAmUfC | SEQ ID | CCUUCAUCCACAAG | SEQ ID |
| | mCfAfCfAfAmGmGmAmU | NO: 1297 | GAUUUUG | NO: 517 |
| | mUmUmUmGm | | | |
| ETXS981 | GmsAmsAmAmGmAmUfC | SEQ ID | GAAAGAUCUCCAUG | SEQ ID |
| | mUfCfCfAfUmGmAmGmG | NO: 1298 | AGGCACG | NO: 518 |
| | mCmAmCmGm | | | |
| ETXS983 | UmsUmsCmAmUmCmCfA | SEQ ID | UUCAUCCACAAGGA | SEQ ID |
| | mCfAfAfGfGmAmUmUmU | NO: 1299 | UUUUGAU | NO: 519 |
| | mUmGmAmUm | | | |
| ETXS985 | UmsUmsUmGmAmUmGfA | SEQ ID | UUUGAUGAGAUUA | SEQ ID |
| | mGfAfUfUfAmAmUmCmC | NO: 1300 | AUCCUGAA | NO: 520 |
| | mUmGmAmAm | | | |
| ETXS987 | UmsUmsGmCmCmUmUfC | SEQ ID | UUGCCUUCAUCCAC | SEQ ID |
| | mAfUfCfCfAmCmAmAmG | NO: 1301 | AAGGAUU | NO: 521 |
| | mGmAmUmUm | | | |
| ETXS989 | GmsAmsUmCmUmCmCfA | SEQ ID | GAUCUCCAUGAGGC | SEQ ID |
| | mUfGfAfGfGmCmAmCmG | NO: 1302 | ACGAUGG | NO: 522 |
| | mAmUmGmGm | | | |
| ETXS991 | UmsGmsCmCmUmUmCfA | SEQ ID | UGCCUUCAUCCACA | SEQ ID |
| | mUfCfCfAfCmAmAmGmG | NO: 1303 | AGGAUUU | NO: 503 |
| | mAmUmUmUm | | | |
| ETXS993 | UmsGmsCmGmAmAmAfG | SEQ ID | UGCGAAAGAUCUCC | SEQ ID |
| | mAfUfCfUfCmCmAmUmG | NO: 1304 | AUGAGGC | NO: 504 |
| | mAmGmGmCm | | | |
| ETXS995 | GmsCmsUmGmGmUmGfG | SEQ ID | GCUGGUGGUCCUCA | SEQ ID |
| | mUfCfCfUfCmAmUmGmG | NO: 1305 | UGGAGAA | NO: 505 |
| | mAmGmAmAm | | | |
| ETXS997 | GmsCmsGmAmAmAmGfA | SEQ ID | GCGAAAGAUCUCCA | SEQ ID |
| | mUfCfUfCfCmAmUmGmA | NO: 1306 | UGAGGCA | NO: 506 |
| | mGmGmCmAm | | | |
| ETXS999 | GmsUmsAmUmGmAmGfA | SEQ ID | GUAUGAGAUGCAU | SEQ ID |
| | mUfGfCfAfUmGmAmGmC | NO: 1307 | GAGCUGCU | NO: 507 |
| | mUmGmCmUm | | | |
| ETXS1001 | GmsUmsUmUmGmAmUfG | SEQ ID | GUUUGAUGAGAUU | SEQ ID |
| | mAfGfAfUfUmAmAmUmC | NO: 1308 | AAUCCUGA | NO: 508 |
| | mCmUmGmAm | | | |
| ETXS1003 | UmsGmsAmGmAmUmUfA | SEQ ID | UGAGAUUAAUCCU | SEQ ID |
| | mAfUfCfCfUmGmAmAmA | NO: 1309 | GAAACCAA | NO: 509 |
| | mCmCmAmAm | | | |
| ETXS1005 | UmsGmsAmUmGmAmGfA | SEQ ID | UGAUGAGAUUAAU | SEQ ID |
| | mUfUfAfAfUmCmCmUmG | NO: 1310 | CCUGAAAC | NO: 510 |
| | mAmAmAmCm | | | |
| ETXS1007 | AmsUmsGmAmGmAmUfU | SEQ ID | AUGAGAUUAAUCC | SEQ ID |
| | mAfAfUfCfCmUmGmAmA | NO: 1311 | UGAAACCA | NO: 511 |
| | mAmCmCmAm | | | |
| ETXS1009 | GmsAmsUmGmAmGmAfU | SEQ ID | GAUGAGAUUAAUC | SEQ ID |
| | mUfAfAfUfCmCmUmGmA | NO: 1312 | CUGAAACC | NO: 512 |
| | mAmAmCmCm | | | |
| ETXS1011 | CmsUmsGmUmUmUmGfA | SEQ ID | CUGUUUGAUGAGA | SEQ ID |
| | mUfGfAfGfAmUmUmAmA | NO: 1313 | UUAAUCCU | NO: 513 |
| | mUmCmCmUm | | | |
| ETXS1013 | UmsUmsUmUmGmCmCfU | SEQ ID | UUUUGCCUUCAUCC | SEQ ID |
| | mUfCfAfUfCmCmAmCmA | NO: 1314 | ACAAGGA | NO: 514 |
| | mAmGmGmAm | | | |
| ETXS1015 | CmsGmsAmAmAmGmAfU | SEQ ID | CGAAAGAUCUCCAU | SEQ ID |
| | mCfUfCfCfAmUmGmAmG | NO: 1315 | GAGGCAC | NO: 515 |
| | mGmCmAmCm | | | |
| ETXS1017 | UmsGmsCmUmGmGmUfG | SEQ ID | UGCUGGUGGUCCUC | SEQ ID |
| | mGfUfCfCfUmCmAmUmG | NO: 1316 | AUGGAGA | NO: 516 |
| | mGmAmGmAm | | | |
| ETXS1019 | CmsCmsUmUmCmAmUfC | SEQ ID | CCUUCAUCCACAAG | SEQ ID |
| | mCfAfCfAfAmGmGmAmU | NO: 1317 | GAUUUUG | NO: 517 |
| | mUmUmUmGm | | | |
| ETXS1021 | GmsAmsAmAmGmAmUfC | SEQ ID | GAAAGAUCUCCAUG | SEQ ID |
| | mUfCfCfAfUmGmAmGmG | NO: 1318 | AGGCACG | NO: 518 |
| | mCmAmCmGm | | | |
| ETXS1023 | UmsUmsCmAmUmCmCfA | SEQ ID | UUCAUCCACAAGGA | SEQ ID |
| | mCfAfAfGfGmAmUmUmU | NO: 1319 | UUUUGAU | NO: 519 |
| | mUmGmAmUm | | | |
| ETXS1025 | UmsUmsUmGmAmUmGfA | SEQ ID | UUUGAUGAGAUUA | SEQ ID |
| | mGfAfUfUfAmAmUmCmC | NO: 1320 | AUCCUGAA | NO: 520 |
| | mUmGmAmAm | | | |
| ETXS1027 | UmsUmsGmCmCmUmUfC | SEQ ID | UUGCCUUCAUCCAC | SEQ ID |
| | mAfUfCfCfAmCmAmAmG | NO: 1321 | AAGGAUU | NO: 521 |
| | mGmAmUmUm | | | |
| ETXS1029 | GmsAmsUmCmUmCmCfA | SEQ ID | GAUCUCCAUGAGGC | SEQ ID |
| | mUfGfAfGfGmCmAmCmG | NO: 1322 | ACGAUGG | NO: 522 |
| | mAmUmGmGm | | | |

Table 26 identifies duplexes with Duplex IDs referencing the modified antisense and sense IDs from previous Tables 24 and 25.

TABLE 26

| Duplex ID | First (Antisense) strand ID | Second (Sense) strand ID |
|---|---|---|
| ETXM116 | ETXS232 | ETXS231 |
| ETXM117 | ETXS234 | ETXS233 |
| ETXM118 | ETXS236 | ETXS235 |
| ETXM119 | ETXS238 | ETXS237 |
| ETXM120 | ETXS240 | ETXS239 |
| ETXM121 | ETXS242 | ETXS241 |
| ETXM122 | ETXS244 | ETXS243 |
| ETXM123 | ETXS246 | ETXS245 |
| ETXM124 | ETXS248 | ETXS247 |
| ETXM125 | ETXS250 | ETXS249 |
| ETXM126 | ETXS252 | ETXS251 |
| ETXM127 | ETXS254 | ETXS253 |
| ETXM128 | ETXS256 | ETXS255 |
| ETXM129 | ETXS258 | ETXS257 |
| ETXM130 | ETXS260 | ETXS259 |
| ETXM131 | ETXS262 | ETXS261 |
| ETXM132 | ETXS264 | ETXS263 |
| ETXM133 | ETXS266 | ETXS265 |
| ETXM134 | ETXS268 | ETXS267 |
| ETXM135 | ETXS270 | ETXS269 |
| ETXM136 | ETXS272 | ETXS271 |
| ETXM137 | ETXS274 | ETXS273 |
| ETXM138 | ETXS276 | ETXS275 |
| ETXM139 | ETXS278 | ETXS277 |
| ETXM140 | ETXS280 | ETXS279 |
| ETXM141 | ETXS282 | ETXS281 |
| ETXM142 | ETXS284 | ETXS283 |
| ETXM143 | ETXS286 | ETXS285 |
| ETXM144 | ETXS288 | ETXS287 |
| ETXM145 | ETXS290 | ETXS289 |
| ETXM146 | ETXS292 | ETXS291 |
| ETXM147 | ETXS294 | ETXS293 |
| ETXM148 | ETXS296 | ETXS295 |
| ETXM149 | ETXS298 | ETXS297 |
| ETXM150 | ETXS300 | ETXS299 |
| ETXM151 | ETXS302 | ETXS301 |
| ETXM152 | ETXS304 | ETXS303 |
| ETXM153 | ETXS306 | ETXS305 |
| ETXM154 | ETXS308 | ETXS307 |
| ETXM155 | ETXS310 | ETXS309 |
| ETXM156 | ETXS312 | ETXS311 |
| ETXM157 | ETXS314 | ETXS313 |
| ETXM158 | ETXS316 | ETXS315 |
| ETXM159 | ETXS318 | ETXS317 |
| ETXM160 | ETXS320 | ETXS319 |
| ETXM161 | ETXS322 | ETXS321 |
| ETXM162 | ETXS324 | ETXS323 |
| ETXM163 | ETXS326 | ETXS325 |
| ETXM164 | ETXS328 | ETXS327 |
| ETXM165 | ETXS330 | ETXS329 |
| ETXM166 | ETXS332 | ETXS331 |
| ETXM167 | ETXS334 | ETXS333 |
| ETXM168 | ETXS336 | ETXS335 |
| ETXM169 | ETXS338 | ETXS337 |
| ETXM170 | ETXS340 | ETXS339 |
| ETXM171 | ETXS342 | ETXS341 |
| ETXM172 | ETXS344 | ETXS343 |
| ETXM173 | ETXS346 | ETXS345 |
| ETXM174 | ETXS348 | ETXS347 |
| ETXM175 | ETXS350 | ETXS349 |
| ETXM176 | ETXS352 | ETXS351 |
| ETXM177 | ETXS354 | ETXS353 |
| ETXM178 | ETXS356 | ETXS355 |
| ETXM179 | ETXS358 | ETXS357 |
| ETXM180 | ETXS360 | ETXS359 |
| ETXM181 | ETXS362 | ETXS361 |
| ETXM182 | ETXS364 | ETXS363 |
| ETXM183 | ETXS366 | ETXS365 |
| ETXM184 | ETXS368 | ETXS367 |
| ETXM185 | ETXS370 | ETXS369 |
| ETXM186 | ETXS372 | ETXS371 |
| ETXM187 | ETXS374 | ETXS373 |
| ETXM188 | ETXS376 | ETXS375 |
| ETXM189 | ETXS378 | ETXS377 |
| ETXM190 | ETXS380 | ETXS379 |
| ETXM191 | ETXS382 | ETXS381 |
| ETXM192 | ETXS384 | ETXS383 |
| ETXM193 | ETXS386 | ETXS385 |
| ETXM194 | ETXS388 | ETXS387 |
| ETXM195 | ETXS390 | ETXS389 |
| ETXM196 | ETXS392 | ETXS391 |
| ETXM197 | ETXS394 | ETXS393 |
| ETXM198 | ETXS396 | ETXS395 |
| ETXM199 | ETXS398 | ETXS397 |
| ETXM200 | ETXS400 | ETXS399 |
| ETXM201 | ETXS402 | ETXS401 |
| ETXM202 | ETXS404 | ETXS403 |
| ETXM203 | ETXS406 | ETXS405 |
| ETXM204 | ETXS408 | ETXS407 |
| ETXM205 | ETXS410 | ETXS409 |
| ETXM206 | ETXS412 | ETXS411 |
| ETXM207 | ETXS414 | ETXS413 |
| ETXM208 | ETXS416 | ETXS415 |
| ETXM209 | ETXS418 | ETXS417 |
| ETXM210 | ETXS420 | ETXS419 |
| ETXM211 | ETXS422 | ETXS421 |
| ETXM212 | ETXS424 | ETXS423 |
| ETXM213 | ETXS426 | ETXS425 |
| ETXM214 | ETXS428 | ETXS427 |
| ETXM215 | ETXS430 | ETXS429 |
| ETXM236 | ETXS472 | ETXS471 |
| ETXM237 | ETXS474 | ETXS473 |
| ETXM238 | ETXS476 | ETXS475 |
| ETXM239 | ETXS478 | ETXS477 |
| ETXM240 | ETXS480 | ETXS479 |
| ETXM241 | ETXS482 | ETXS481 |
| ETXM242 | ETXS484 | ETXS483 |
| ETXM243 | ETXS486 | ETXS485 |
| ETXM244 | ETXS488 | ETXS487 |
| ETXM245 | ETXS490 | ETXS489 |
| ETXM246 | ETXS492 | ETXS491 |
| ETXM247 | ETXS494 | ETXS493 |
| ETXM248 | ETXS496 | ETXS495 |
| ETXM249 | ETXS498 | ETXS497 |
| ETXM250 | ETXS500 | ETXS499 |
| ETXM251 | ETXS502 | ETXS501 |
| ETXM252 | ETXS504 | ETXS503 |
| ETXM253 | ETXS506 | ETXS505 |
| ETXM254 | ETXS508 | ETXS507 |
| ETXM255 | ETXS510 | ETXS509 |
| ETXM256 | ETXS512 | ETXS511 |
| ETXM257 | ETXS514 | ETXS513 |
| ETXM258 | ETXS516 | ETXS515 |
| ETXM259 | ETXS518 | ETXS517 |
| ETXM260 | ETXS520 | ETXS519 |
| ETXM261 | ETXS522 | ETXS521 |
| ETXM262 | ETXS524 | ETXS523 |
| ETXM263 | ETXS526 | ETXS525 |
| ETXM264 | ETXS528 | ETXS527 |
| ETXM265 | ETXS530 | ETXS529 |
| ETXM266 | ETXS532 | ETXS531 |
| ETXM267 | ETXS534 | ETXS533 |
| ETXM268 | ETXS536 | ETXS535 |
| ETXM269 | ETXS538 | ETXS537 |
| ETXM270 | ETXS540 | ETXS539 |
| ETXM271 | ETXS542 | ETXS541 |
| ETXM272 | ETXS544 | ETXS543 |
| ETXM273 | ETXS546 | ETXS545 |
| ETXM274 | ETXS548 | ETXS547 |
| ETXM275 | ETXS550 | ETXS549 |
| ETXM276 | ETXS552 | ETXS551 |
| ETXM277 | ETXS554 | ETXS553 |
| ETXM278 | ETXS556 | ETXS555 |
| ETXM279 | ETXS558 | ETXS557 |
| ETXM280 | ETXS560 | ETXS559 |
| ETXM281 | ETXS562 | ETXS561 |
| ETXM282 | ETXS564 | ETXS563 |
| ETXM283 | ETXS566 | ETXS565 |
| ETXM284 | ETXS568 | ETXS567 |
| ETXM285 | ETXS570 | ETXS569 |
| ETXM286 | ETXS572 | ETXS571 |
| ETXM287 | ETXS574 | ETXS573 |
| ETXM288 | ETXS576 | ETXS575 |
| ETXM289 | ETXS578 | ETXS577 |
| ETXM290 | ETXS580 | ETXS579 |
| ETXM291 | ETXS582 | ETXS581 |
| ETXM292 | ETXS584 | ETXS583 |
| ETXM293 | ETXS586 | ETXS585 |
| ETXM294 | ETXS588 | ETXS587 |
| ETXM295 | ETXS590 | ETXS589 |
| ETXM296 | ETXS592 | ETXS591 |
| ETXM297 | ETXS594 | ETXS593 |
| ETXM298 | ETXS596 | ETXS595 |
| ETXM299 | ETXS598 | ETXS597 |
| ETXM300 | ETXS600 | ETXS599 |
| ETXM301 | ETXS602 | ETXS601 |
| ETXM302 | ETXS604 | ETXS603 |
| ETXM303 | ETXS606 | ETXS605 |
| ETXM304 | ETXS608 | ETXS607 |
| ETXM305 | ETXS610 | ETXS609 |
| ETXM306 | ETXS612 | ETXS611 |
| ETXM307 | ETXS614 | ETXS613 |
| ETXM308 | ETXS616 | ETXS615 |
| ETXM309 | ETXS618 | ETXS617 |
| ETXM310 | ETXS620 | ETXS619 |
| ETXM311 | ETXS622 | ETXS621 |
| ETXM312 | ETXS624 | ETXS623 |
| ETXM313 | ETXS626 | ETXS625 |
| ETXM314 | ETXS628 | ETXS627 |
| ETXM315 | ETXS630 | ETXS629 |
| ETXM316 | ETXS632 | ETXS631 |
| ETXM317 | ETXS634 | ETXS633 |
| ETXM318 | ETXS636 | ETXS635 |
| ETXM319 | ETXS638 | ETXS637 |
| ETXM320 | ETXS640 | ETXS639 |
| ETXM321 | ETXS642 | ETXS641 |
| ETXM322 | ETXS644 | ETXS643 |
| ETXM323 | ETXS646 | ETXS645 |
| ETXM324 | ETXS648 | ETXS647 |
| ETXM325 | ETXS650 | ETXS649 |
| ETXM326 | ETXS652 | ETXS651 |
| ETXM327 | ETXS654 | ETXS653 |
| ETXM328 | ETXS656 | ETXS655 |
| ETXM329 | ETXS658 | ETXS657 |
| ETXM330 | ETXS660 | ETXS659 |
| ETXM331 | ETXS662 | ETXS661 |
| ETXM332 | ETXS664 | ETXS663 |
| ETXM333 | ETXS666 | ETXS665 |
| ETXM334 | ETXS668 | ETXS667 |
| ETXM335 | ETXS670 | ETXS669 |
| ETXM336 | ETXS672 | ETXS671 |
| ETXM337 | ETXS674 | ETXS673 |
| ETXM338 | ETXS676 | ETXS675 |
| ETXM339 | ETXS678 | ETXS677 |
| ETXM340 | ETXS680 | ETXS679 |
| ETXM341 | ETXS682 | ETXS681 |
| ETXM342 | ETXS684 | ETXS683 |
| ETXM343 | ETXS686 | ETXS685 |
| ETXM344 | ETXS688 | ETXS687 |
| ETXM345 | ETXS690 | ETXS689 |
| ETXM346 | ETXS692 | ETXS691 |
| ETXM347 | ETXS694 | ETXS693 |
| ETXM348 | ETXS696 | ETXS695 |
| ETXM349 | ETXS698 | ETXS697 |
| ETXM350 | ETXS700 | ETXS699 |
| ETXM351 | ETXS702 | ETXS701 |
| ETXM352 | ETXS704 | ETXS703 |
| ETXM353 | ETXS706 | ETXS705 |
| ETXM354 | ETXS708 | ETXS707 |
| ETXM355 | ETXS710 | ETXS709 |
| ETXM356 | ETXS712 | ETXS711 |
| ETXM357 | ETXS714 | ETXS713 |
| ETXM358 | ETXS716 | ETXS715 |
| ETXM359 | ETXS718 | ETXS717 |
| ETXM360 | ETXS720 | ETXS719 |
| ETXM361 | ETXS722 | ETXS721 |
| ETXM362 | ETXS724 | ETXS723 |
| ETXM363 | ETXS726 | ETXS725 |
| ETXM364 | ETXS728 | ETXS727 |
| ETXM365 | ETXS730 | ETXS729 |
| ETXM366 | ETXS732 | ETXS731 |
| ETXM367 | ETXS734 | ETXS733 |
| ETXM368 | ETXS736 | ETXS735 |
| ETXM369 | ETXS738 | ETXS737 |
| ETXM370 | ETXS740 | ETXS739 |
| ETXM371 | ETXS742 | ETXS741 |
| ETXM372 | ETXS744 | ETXS743 |
| ETXM373 | ETXS746 | ETXS745 |
| ETXM374 | ETXS748 | ETXS747 |
| ETXM375 | ETXS750 | ETXS749 |
| ETXM376 | ETXS752 | ETXS751 |
| ETXM377 | ETXS754 | ETXS753 |
| ETXM378 | ETXS756 | ETXS755 |
| ETXM379 | ETXS758 | ETXS757 |
| ETXM380 | ETXS760 | ETXS759 |
| ETXM381 | ETXS762 | ETXS761 |
| ETXM382 | ETXS764 | ETXS763 |
| ETXM383 | ETXS766 | ETXS765 |
| ETXM384 | ETXS768 | ETXS767 |
| ETXM385 | ETXS770 | ETXS769 |
| ETXM386 | ETXS772 | ETXS771 |
| ETXM387 | ETXS774 | ETXS773 |
| ETXM388 | ETXS776 | ETXS775 |
| ETXM389 | ETXS778 | ETXS777 |
| ETXM390 | ETXS780 | ETXS779 |
| ETXM391 | ETXS782 | ETXS781 |
| ETXM392 | ETXS784 | ETXS783 |
| ETXM393 | ETXS786 | ETXS785 |
| ETXM394 | ETXS788 | ETXS787 |
| ETXM395 | ETXS790 | ETXS789 |
| ETXM396 | ETXS792 | ETXS791 |
| ETXM397 | ETXS794 | ETXS793 |
| ETXM398 | ETXS796 | ETXS795 |
| ETXM399 | ETXS798 | ETXS797 |
| ETXM400 | ETXS800 | ETXS799 |
| ETXM401 | ETXS802 | ETXS801 |
| ETXM402 | ETXS804 | ETXS803 |
| ETXM403 | ETXS806 | ETXS805 |
| ETXM404 | ETXS808 | ETXS807 |
| ETXM405 | ETXS810 | ETXS809 |
| ETXM406 | ETXS812 | ETXS811 |
| ETXM407 | ETXS814 | ETXS813 |
| ETXM408 | ETXS816 | ETXS815 |
| ETXM409 | ETXS818 | ETXS817 |
| ETXM410 | ETXS820 | ETXS819 |
| ETXM411 | ETXS822 | ETXS821 |
| ETXM412 | ETXS824 | ETXS823 |
| ETXM413 | ETXS826 | ETXS825 |
| ETXM414 | ETXS828 | ETXS827 |
| ETXM415 | ETXS830 | ETXS829 |
| ETXM436 | ETXS872 | ETXS871 |
| ETXM437 | ETXS874 | ETXS873 |
| ETXM438 | ETXS876 | ETXS875 |
| ETXM439 | ETXS878 | ETXS877 |
| ETXM440 | ETXS880 | ETXS879 |
| ETXM441 | ETXS882 | ETXS881 |
| ETXM442 | ETXS884 | ETXS883 |
| ETXM443 | ETXS886 | ETXS885 |
| ETXM444 | ETXS888 | ETXS887 |
| ETXM445 | ETXS890 | ETXS889 |
| ETXM446 | ETXS892 | ETXS891 |
| ETXM447 | ETXS894 | ETXS893 |
| ETXM448 | ETXS896 | ETXS895 |
| ETXM449 | ETXS898 | ETXS897 |
| ETXM450 | ETXS900 | ETXS899 |
| ETXM451 | ETXS902 | ETXS901 |
| ETXM452 | ETXS904 | ETXS903 |
| ETXM453 | ETXS906 | ETXS905 |
| ETXM454 | ETXS908 | ETXS907 |
| ETXM455 | ETXS910 | ETXS909 |
| ETXM456 | ETXS912 | ETXS911 |
| ETXM457 | ETXS914 | ETXS913 |
| ETXM458 | ETXS916 | ETXS915 |
| ETXM459 | ETXS918 | ETXS917 |
| ETXM460 | ETXS920 | ETXS919 |
| ETXM461 | ETXS922 | ETXS921 |
| ETXM462 | ETXS924 | ETXS923 |
| ETXM463 | ETXS926 | ETXS925 |
| ETXM464 | ETXS928 | ETXS927 |
| ETXM465 | ETXS930 | ETXS929 |
| ETXM466 | ETXS932 | ETXS931 |
| ETXM467 | ETXS934 | ETXS933 |
| ETXM468 | ETXS936 | ETXS935 |
| ETXM469 | ETXS938 | ETXS937 |
| ETXM470 | ETXS940 | ETXS939 |
| ETXM471 | ETXS942 | ETXS941 |
| ETXM472 | ETXS944 | ETXS943 |
| ETXM473 | ETXS946 | ETXS945 |
| ETXM474 | ETXS948 | ETXS947 |
| ETXM475 | ETXS950 | ETXS949 |
| ETXM476 | ETXS952 | ETXS951 |
| ETXM477 | ETXS954 | ETXS953 |
| ETXM478 | ETXS956 | ETXS955 |
| ETXM479 | ETXS958 | ETXS957 |
| ETXM480 | ETXS960 | ETXS959 |
| ETXM481 | ETXS962 | ETXS961 |
| ETXM482 | ETXS964 | ETXS963 |
| ETXM483 | ETXS966 | ETXS965 |
| ETXM484 | ETXS968 | ETXS967 |
| ETXM485 | ETXS970 | ETXS969 |
| ETXM486 | ETXS972 | ETXS971 |
| ETXM487 | ETXS974 | ETXS973 |
| ETXM488 | ETXS976 | ETXS975 |
| ETXM489 | ETXS978 | ETXS977 |
| ETXM490 | ETXS980 | ETXS979 |
| ETXM491 | ETXS982 | ETXS981 |
| ETXM492 | ETXS984 | ETXS983 |
| ETXM493 | ETXS986 | ETXS985 |
| ETXM494 | ETXS988 | ETXS987 |
| ETXM495 | ETXS990 | ETXS989 |
| ETXM496 | ETXS992 | ETXS991 |
| ETXM497 | ETXS994 | ETXS993 |
| ETXM498 | ETXS996 | ETXS995 |
| ETXM499 | ETXS998 | ETXS997 |
| ETXM500 | ETXS1000 | ETXS999 |
| ETXM501 | ETXS1002 | ETXS1001 |
| ETXM502 | ETXS1004 | ETXS1003 |
| ETXM503 | ETXS1006 | ETXS1005 |
| ETXM504 | ETXS1008 | ETXS1007 |
| ETXM505 | ETXS1010 | ETXS1009 |
| ETXM506 | ETXS1012 | ETXS1011 |
| ETXM507 | ETXS1014 | ETXS1013 |
| ETXM508 | ETXS1016 | ETXS1015 |
| ETXM509 | ETXS1018 | ETXS1017 |
| ETXM510 | ETXS1020 | ETXS1019 |
| ETXM511 | ETXS1022 | ETXS1021 |
| ETXM512 | ETXS1024 | ETXS1023 |
| ETXM513 | ETXS1026 | ETXS1025 |
| ETXM514 | ETXS1028 | ETXS1027 |
| ETXM515 | ETXS1030 | ETXS1029 |

Definitions as provided in the above Tables:
A—adenosine
C—cytidine
G—guanosine
T—thymidine
m—2′-O-methyl
f—2′fluro
s—phosphorothioate bond

### Example 13: Inhibition Screen for HCII and ZPI Expression in Human Huh7 Cells

HCII: Huh7 cells (human hepatocyte-derived cell line, obtained from JCRB Cell Bank) were maintained in Dulbecco's Modified Eagle Medium (DMEM) supplemented with 10% FBS at 37° C. in at atmosphere of 5% CO₂. Cells were transfected with siRNA duplexes targeting HCII mRNA or a negative control siRNA (siRNA-control; sense strand 5′-UUCUCCGAACGUGUCACGUTT-3′, antisense strand 5′-ACGUGACACGUUCGGAGAATT-3′) at a final duplex concentration of 5 nM and 0.1 nM. Transfection was carried out by adding 9.7 μL Opti-MEM (ThermoFisher) plus 0.3 μL Lipofectamine RNAiMAX (ThermoFisher) to 10 μL of each siRNA duplex. The mixture was incubated at room temperature for 15 minutes before being added to 100 μL of complete growth medium containing 20,000 Huh7 cells. Cells were incubated for 24 hours at 37° C./5% CO₂ prior to total RNA purification using a RNeasy 96 Kit (Qiagen). Each duplex was tested by transfection in duplicate wells in two independent experiments.

cDNA synthesis was performed using FastQuant RT (with gDNase) Kit (Tiangen). Real-time quantitative PCR (qPCR) was performed on an ABI Prism 7900HT or ABI QuantStudio 7 with primers specific for human HCII (Hs00164821_m1) and human GAPDH (Hs02786624_g1) using FastStart Universal Probe Master Kit (Roche).

qPCR was performed in duplicate on cDNA derived from each well and the mean Ct calculated. Relative HCII expression was calculated from mean Ct values using the comparative Ct (ΔΔCt) method, normalised to GAPDH and relative to untreated cells. Based on the results of primary screen, siRNA duplexes displaying good activity were selected for dose-response follow-up. Results are shown in FIG. 45. Sequences of RNAi molecules are depicted herein.

ZPI: Huh7 cells (human hepatocyte-derived cell line, obtained from JCRB Cell Bank) were maintained in Dulbecco's Modified Eagle Medium (DMEM) supplemented with 10% FBS at 37° C. in at atmosphere of 5% CO₂. Cells were transfected with siRNA duplexes targeting ZPI mRNA or a negative control siRNA (siRNA-control; sense strand 5′-UUCUCCGAACGUGUCACGUTT-3′, antisense strand 5′-ACGUGACACGUUCGGAGAATT-3′) at a final duplex concentration of 10 nM and 0.1 nM. Transfection was carried out by adding 9.7 μL Opti-MEM (ThermoFisher) plus 0.3 μL Lipofectamine RNAiMAX (ThermoFisher) to 10 μL of each siRNA duplex. The mixture was incubated at room temperature for 15 minutes before being added to 100 μL of complete growth medium containing 20,000 Huh7 cells. Cells were incubated for 24 hours at 37° C./5% CO₂ prior to total RNA purification using a RNeasy 96 Kit (Qiagen). Each duplex was tested by transfection in duplicate wells in two independent experiments.

cDNA synthesis was performed using FastQuant RT (with gDNase) Kit (Tiangen). Real-time quantitative PCR (qPCR) was performed on an ABI Prism 7900HT or ABI QuantStudio 7 with primers specific for human ZPI (Hs01547819_m1) and human GAPDH (Hs02786624_g1) using FastStart Universal Probe Master Kit (Roche).

qPCR was performed in duplicate on cDNA derived from each well and the mean Ct calculated. Relative ZPI expression was calculated from mean Ct values using the comparative Ct (ΔΔCt) method, normalised to GAPDH and relative to untreated cells. Based on the results of primary screen, siRNA duplexes displaying good activity were selected for dose-response follow-up. Results are shown in FIG. 45. Sequences of RNAi molecules are depicted herein.

### Example 14: Dose-Response for Inhibition of HCII in Human Huh7 Cells

Huh7 cells (human hepatocyte-derived cell line, obtained from JCRB Cell Bank) were maintained in Dulbecco's Modified Eagle Medium (DMEM) supplemented with 10% FBS at 37° C. in at atmosphere of 5% CO₂. Cells were transfected with siRNA duplexes targeting HCII mRNA or a negative control siRNA (siRNA-control; sense strand 5′-UUCUCCGAACGUGUCACGUTT-3′, antisense strand 5′-ACGUGACACGUUCGGAGAATT-3′) using 10×3-fold serial dilutions over a final duplex concentration range of 20 nM to 1 μM. Transfection was carried out by adding 9.7 μL Opti-MEM (ThermoFisher) plus 0.3 μL Lipofectamine RNAiMAX (ThermoFisher) to 10 μL of each siRNA duplex. The mixture was incubated at room temperature for 15 minutes before being added to 100 μL of complete growth medium containing 20,000 Huh7 cells. Cells were incubated for 24 hours at 37° C./5% CO₂ prior to total RNA purification using a RNeasy 96 Kit (Qiagen). Each duplex was tested by transfection in duplicate wells in a single experiment.

cDNA synthesis was performed using FastQuant RT (with gDNase) Kit (Tiangen). Real-time quantitative PCR (qPCR) was performed on an ABI Prism 7900HT or ABI QuantStudio 7 with primers specific for human HCII (Hs00164821_m1) and human GAPDH (Hs02786624_g1) using FastStart Universal Probe Master Kit (Roche).

qPCR was performed in duplicate on cDNA derived from each well and the mean Ct calculated. Relative HCII expression was calculated from mean Ct values using the comparative Ct (ΔΔCt) method, normalised to GAPDH and relative to untreated cells. Maximum percent inhibition of HCII expression and IC50 values were calculated using a four parameter (variable slope) model using GraphPad Prism 9. Results are shown in FIG. 45. Sequences of RNAi molecules are depicted in the relevant Tables herein.

### Dose-Response for Inhibition of ZPI Expression in Human Huh7 Cells

Huh7 cells (human hepatocyte-derived cell line, obtained from JCRB Cell Bank) were maintained in Dulbecco's Modified Eagle Medium (DMEM) supplemented with 10% FBS at 37° C. in at atmosphere of 5% CO₂. Cells were transfected with siRNA duplexes targeting ZPI mRNA or a negative control siRNA (siRNA-control; sense strand 5′-UUCUCCGAACGUGUCACGUTT-3′, antisense strand 5′-ACGUGACACGUUCGGAGAATT-3′) using 10×3-fold serial dilutions over a final duplex concentration range of 20 nM to 1 μM. Transfection was carried out by adding 9.7 μL Opti-MEM (ThermoFisher) plus 0.3 μL Lipofectamine RNAiMAX (ThermoFisher) to 10 μL of each siRNA duplex. The mixture was incubated at room temperature for 15 minutes before being added to 100 μL of complete growth medium containing 20,000 Huh7 cells. Cells were incubated for 24 hours at 37° C./5% CO₂ prior to total RNA purification using a RNeasy 96 Kit (Qiagen). Each duplex was tested by transfection in duplicate wells in a single experiment.

cDNA synthesis was performed using FastQuant RT (with gDNase) Kit (Tiangen). Real-time quantitative PCR (qPCR) was performed on an ABI Prism 7900HT or ABI QuantStudio 7 with primers specific for human ZPI (Hs01547819_m1) and human GAPDH (Hs02786624_g1) using FastStart Universal Probe Master Kit (Roche).

qPCR was performed in duplicate on cDNA derived from each well and the mean Ct calculated. Relative ZPI expression was calculated from mean Ct values using the comparative Ct (ΔΔCt) method, normalised to GAPDH and relative to untreated cells. Maximum percent inhibition of ZPI expression and IC50 values were calculated using a four parameter (variable slope) model using GraphPad Prism 9. Results are shown in FIG. 45. Sequences of RNAi molecules are depicted in the relevant Tables herein.

TABLE 27
Relative mRNA Expression

| | | | | | Mean | Mean | |
|---|---|---|---|---|---|---|---|
| | | | | | Rel- | Rel- | |
| | | | | | ative | ative | |
| | | | | SEQ | Ex- | Ex- | |
| | | SEQ ID | | ID | pres- | pres- | |
| | | NO | | NO | sion/ | sion/ | |
| Duplex | Antisense | (AS- | Sense | (SS- | 0.1 | 1 | Tar- |
| ID | strand ID | mod) | strand ID | mod) | nM | nM | get |
| ETXM116 | ETXS232 | SEQ ID | ETXS231 | SEQ ID | 0.93 | 0.4 | HCII |
| | | NO: 603 | | NO: 963 | | | |
| ETXM117 | ETXS234 | SEQ ID | ETXS233 | SEQ ID | 1 | 0.47 | HCII |
| | | NO: 604 | | NO: 964 | | | |
| ETXM118 | ETXS236 | SEQ ID | ETXS235 | SEQ ID | 0.95 | 0.42 | HCII |
| | | NO: 605 | | NO: 965 | | | |
| ETXM119 | ETXS238 | SEQ ID | ETXS237 | SEQ ID | 1.03 | 0.86 | HCII |
| | | NO: 606 | | NO: 966 | | | |
| ETXM120 | ETXS240 | SEQ ID | ETXS239 | SEQ ID | 0.95 | 0.6 | HCII |
| | | NO: 607 | | NO: 967 | | | |
| ETXM121 | ETXS242 | SEQ ID | ETXS241 | SEQ ID | 0.89 | 0.54 | HCII |
| | | NO: 608 | | NO: 968 | | | |
| ETXM122 | ETXS244 | SEQ ID | ETXS243 | SEQ ID | 1.01 | 0.77 | HCII |
| | | NO: 609 | | NO: 969 | | | |
| ETXM123 | ETXS246 | SEQ ID | ETXS245 | SEQ ID | 1.03 | 0.73 | HCII |
| | | NO: 610 | | NO: 970 | | | |
| ETXM124 | ETXS248 | SEQ ID | ETXS247 | SEQ ID | 0.84 | 0.38 | HCII |
| | | NO: 611 | | NO: 971 | | | |
| ETXM125 | ETXS250 | SEQ ID | ETXS249 | SEQ ID | 0.88 | 0.6 | HCII |
| | | NO: 612 | | NO: 972 | | | |
| ETXM126 | ETXS252 | SEQ ID | ETXS251 | SEQ ID | 0.68 | 0.31 | HCII |
| | | NO: 613 | | NO: 973 | | | |
| ETXM127 | ETXS254 | SEQ ID | ETXS253 | SEQ ID | 0.84 | 0.57 | HCII |
| | | NO: 614 | | NO: 974 | | | |
| ETXM128 | ETXS256 | SEQ ID | ETXS255 | SEQ ID | 0.67 | 0.36 | HCII |
| | | NO: 615 | | NO: 975 | | | |
| ETXM129 | ETXS258 | SEQ ID | ETXS257 | SEQ ID | 0.62 | 0.31 | HCII |
| | | NO: 616 | | NO: 976 | | | |
| ETXM130 | ETXS260 | SEQ ID | ETXS259 | SEQ ID | 0.59 | 0.26 | HCII |
| | | NO: 617 | | NO: 977 | | | |
| ETXM131 | ETXS262 | SEQ ID | ETXS261 | SEQ ID | 0.7 | 0.34 | HCII |
| | | NO: 618 | | NO: 978 | | | |
| ETXM132 | ETXS264 | SEQ ID | ETXS263 | SEQ ID | 0.48 | 0.22 | HCII |
| | | NO: 619 | | NO: 979 | | | |
| ETXM133 | ETXS266 | SEQ ID | ETXS265 | SEQ ID | 0.72 | 0.32 | HCII |
| | | NO: 620 | | NO: 980 | | | |
| ETXM134 | ETXS268 | SEQ ID | ETXS267 | SEQ ID | 0.66 | 0.28 | HCII |
| | | NO: 621 | | NO: 981 | | | |
| ETXM135 | ETXS270 | SEQ ID | ETXS269 | SEQ ID | 0.74 | 0.41 | HCII |
| | | NO: 622 | | NO: 982 | | | |
| ETXM136 | ETXS272 | SEQ ID | ETXS271 | SEQ ID | 0.98 | 1 | HCII |
| | | NO: 623 | | NO: 983 | | | |
| ETXM137 | ETXS274 | SEQ ID | ETXS273 | SEQ ID | 0.89 | 0.71 | HCII |
| | | NO: 624 | | NO: 984 | | | |
| ETXM138 | ETXS276 | SEQ ID | ETXS275 | SEQ ID | 0.87 | 0.78 | HCII |
| | | NO: 625 | | NO: 985 | | | |
| ETXM139 | ETXS278 | SEQ ID | ETXS277 | SEQ ID | 0.89 | 0.65 | HCII |
| | | NO: 626 | | NO: 986 | | | |
| ETXM140 | ETXS280 | SEQ ID | ETXS279 | SEQ ID | 0.81 | 0.32 | HCII |
| | | NO: 627 | | NO: 987 | | | |
| ETXM141 | ETXS282 | SEQ ID | ETXS281 | SEQ ID | 0.91 | 0.78 | HCII |
| | | NO: 628 | | NO: 988 | | | |
| ETXM142 | ETXS284 | SEQ ID | ETXS283 | SEQ ID | 0.82 | 0.46 | HCII |
| | | NO: 629 | | NO: 989 | | | |
| ETXM143 | ETXS286 | SEQ ID | ETXS285 | SEQ ID | 0.95 | 0.85 | HCII |
| | | NO: 630 | | NO: 990 | | | |
| ETXM144 | ETXS288 | SEQ ID | ETXS287 | SEQ ID | 0.93 | 0.55 | HCII |
| | | NO: 631 | | NO: 991 | | | |
| ETXM145 | ETXS290 | SEQ ID | ETXS289 | SEQ ID | 0.58 | 0.16 | HCII |
| | | NO: 632 | | NO: 992 | | | |
| ETXM146 | ETXS292 | SEQ ID | ETXS291 | SEQ ID | 0.44 | 0.2 | HCII |
| | | NO: 633 | | NO: 993 | | | |
| ETXM147 | ETXS294 | SEQ ID | ETXS293 | SEQ ID | 0.8 | 0.44 | HCII |
| | | NO: 634 | | NO: 994 | | | |
| ETXM148 | ETXS296 | SEQ ID | ETXS295 | SEQ ID | 0.85 | 0.73 | HCII |
| | | NO: 635 | | NO: 995 | | | |
| ETXM149 | ETXS298 | SEQ ID | ETXS297 | SEQ ID | 0.81 | 0.36 | HCII |
| | | NO: 636 | | NO: 996 | | | |
| ETXM150 | ETXS300 | SEQ ID | ETXS299 | SEQ ID | 0.79 | 0.59 | HCII |
| | | NO: 637 | | NO: 997 | | | |
| ETXM151 | ETXS302 | SEQ ID | ETXS301 | SEQ ID | 0.93 | 0.91 | HCII |
| | | NO: 638 | | NO: 998 | | | |
| ETXM152 | ETXS304 | SEQ ID | ETXS303 | SEQ ID | 0.86 | 0.79 | HCII |
| | | NO: 639 | | NO: 999 | | | |
| ETXM153 | ETXS306 | SEQ ID | ETXS305 | SEQ ID | 0.71 | 0.46 | HCII |
| | | NO: 640 | | NO: | | | |
| | | | | 1000 | | | |
| ETXM154 | ETXS308 | SEQ ID | ETXS307 | SEQ ID | 0.66 | 0.38 | HCII |
| | | NO: 641 | | NO: | | | |
| | | | | 1001 | | | |
| ETXM155 | ETXS310 | SEQ ID | ETXS309 | SEQ ID | 0.7 | 0.43 | HCII |
| | | NO: 642 | | NO: | | | |
| | | | | 1002 | | | |
| ETXM156 | ETXS312 | SEQ ID | ETXS311 | SEQ ID | 0.63 | 0.25 | HCII |
| | | NO: 643 | | NO: | | | |
| | | | | 1003 | | | |
| ETXM157 | ETXS314 | SEQ ID | ETXS313 | SEQ ID | 0.86 | 0.7 | HCII |
| | | NO: 644 | | NO: | | | |
| | | | | 1004 | | | |
| ETXM158 | ETXS316 | SEQ ID | ETXS315 | SEQ ID | 0.76 | 0.5 | HCII |
| | | NO: 645 | | NO: | | | |
| | | | | 1005 | | | |
| ETXM159 | ETXS318 | SEQ ID | ETXS317 | SEQ ID | 0.71 | 0.49 | HCII |
| | | NO: 646 | | NO: | | | |
| | | | | 1006 | | | |
| ETXM160 | ETXS320 | SEQ ID | ETXS319 | SEQ ID | 0.54 | 0.2 | HCII |
| | | NO: 647 | | NO: | | | |
| | | | | 1007 | | | |
| ETXM161 | ETXS322 | SEQ ID | ETXS321 | SEQ ID | 1.15 | 0.7 | HCII |
| | | NO: 648 | | NO: | | | |
| | | | | 1008 | | | |
| ETXM162 | ETXS324 | SEQ ID | ETXS323 | SEQ ID | 1.17 | 1.24 | HCII |
| | | NO: 649 | | NO: | | | |
| | | | | 1009 | | | |
| ETXM163 | ETXS326 | SEQ ID | ETXS325 | SEQ ID | 1.13 | 1.14 | HCII |
| | | NO: 650 | | NO: | | | |
| | | | | 1010 | | | |
| ETXM164 | ETXS328 | SEQ ID | ETXS327 | SEQ ID | 1.12 | 1.15 | HCII |
| | | NO: 651 | | NO: | | | |
| | | | | 1011 | | | |
| ETXM165 | ETXS330 | SEQ ID | ETXS329 | SEQ ID | 1.02 | 0.69 | HCII |
| | | NO: 652 | | NO: | | | |
| | | | | 1012 | | | |
| ETXM166 | ETXS332 | SEQ ID | ETXS331 | SEQ ID | 0.78 | 0.41 | HCII |
| | | NO: 653 | | NO: | | | |
| | | | | 1013 | | | |
| ETXM167 | ETXS334 | SEQ ID | ETXS333 | SEQ ID | 0.72 | 0.48 | HCII |
| | | NO: 654 | | NO: | | | |
| | | | | 1014 | | | |
| ETXM168 | ETXS336 | SEQ ID | ETXS335 | SEQ ID | 0.74 | 0.5 | HCII |
| | | NO: 655 | | NO: | | | |
| | | | | 1015 | | | |
| ETXM169 | ETXS338 | SEQ ID | ETXS337 | SEQ ID | 0.54 | 0.33 | HCII |
| | | NO: 656 | | NO: | | | |
| | | | | 1016 | | | |
| ETXM170 | ETXS340 | SEQ ID | ETXS339 | SEQ ID | 0.61 | 0.36 | HCII |
| | | NO: 657 | | NO: | | | |
| | | | | 1017 | | | |
| ETXM171 | ETXS342 | SEQ ID | ETXS341 | SEQ ID | 0.86 | 0.41 | HCII |
| | | NO: 658 | | NO: | | | |
| | | | | 1018 | | | |
| ETXM172 | ETXS344 | SEQ ID | ETXS343 | SEQ ID | 0.95 | 0.67 | HCII |
| | | NO: 659 | | NO: | | | |
| | | | | 1019 | | | |
| ETXM173 | ETXS346 | SEQ ID | ETXS345 | SEQ ID | 0.84 | 0.47 | HCII |
| | | NO: 660 | | NO: | | | |
| | | | | 1020 | | | |
| ETXM174 | ETXS348 | SEQ ID | ETXS347 | SEQ ID | 0.82 | 0.69 | HCII |
| | | NO: 661 | | NO: | | | |
| | | | | 1021 | | | |
| ETXM175 | ETXS350 | SEQ ID | ETXS349 | SEQ ID | 0.97 | 0.71 | HCII |
| | | NO: 662 | | NO: | | | |
| | | | | 1022 | | | |
| ETXM176 | ETXS352 | SEQ ID | ETXS351 | SEQ ID | 0.93 | 0.78 | HCII |
| | | NO: 663 | | NO: | | | |
| | | | | 1023 | | | |
| ETXM177 | ETXS354 | SEQ ID | ETXS353 | SEQ ID | 0.86 | 0.81 | HCII |
| | | NO: 664 | | NO: | | | |
| | | | | 1024 | | | |
| ETXM178 | ETXS356 | SEQ ID | ETXS355 | SEQ ID | 0.68 | 0.4 | HCII |
| | | NO: 665 | | NO: | | | |
| | | | | 1025 | | | |
| ETXM179 | ETXS358 | SEQ ID | ETXS357 | SEQ ID | 0.68 | 0.4 | HCII |
| | | NO: 666 | | NO: | | | |
| | | | | 1026 | | | |
| ETXM180 | ETXS360 | SEQ ID | ETXS359 | SEQ ID | 0.28 | 0.13 | HCII |
| | | NO: 667 | | NO: | | | |
| | | | | 1027 | | | |
| ETXM181 | ETXS362 | SEQ ID | ETXS361 | SEQ ID | 1.04 | 1.04 | HCII |
| | | NO: 668 | | NO: | | | |
| | | | | 1028 | | | |
| ETXM182 | ETXS364 | SEQ ID | ETXS363 | SEQ ID | 0.43 | 0.23 | HCII |
| | | NO: 669 | | NO: | | | |
| | | | | 1029 | | | |
| ETXM183 | ETXS366 | SEQ ID | ETXS365 | SEQ ID | 0.96 | 0.83 | HCII |
| | | NO: 670 | | NO: | | | |
| | | | | 1030 | | | |
| ETXM184 | ETXS368 | SEQ ID | ETXS367 | SEQ ID | 0.97 | 0.78 | HCII |
| | | NO: 671 | | NO: | | | |
| | | | | 1031 | | | |
| ETXM185 | ETXS370 | SEQ ID | ETXS369 | SEQ ID | 1.03 | 0.88 | HCII |
| | | NO: 672 | | NO: | | | |
| | | | | 1032 | | | |
| ETXM186 | ETXS372 | SEQ ID | ETXS371 | SEQ ID | 0.94 | 0.71 | HCII |
| | | NO: 673 | | NO: | | | |
| | | | | 1033 | | | |
| ETXM187 | ETXS374 | SEQ ID | ETXS373 | SEQ ID | 0.92 | 0.68 | HCII |
| | | NO: 674 | | NO: | | | |
| | | | | 1034 | | | |
| ETXM188 | ETXS376 | SEQ ID | ETXS375 | SEQ ID | 0.68 | 0.3 | HCII |
| | | NO: 675 | | NO: | | | |
| | | | | 1035 | | | |
| ETXM189 | ETXS378 | SEQ ID | ETXS377 | SEQ ID | 0.76 | 0.66 | HCII |
| | | NO: 676 | | NO: | | | |
| | | | | 1036 | | | |
| ETXM190 | ETXS380 | SEQ ID | ETXS379 | SEQ ID | 0.81 | 0.48 | HCII |
| | | NO: 677 | | NO: | | | |
| | | | | 1037 | | | |
| ETXM191 | ETXS382 | SEQ ID | ETXS381 | SEQ ID | 0.86 | 0.42 | HCII |
| | | NO: 678 | | NO: | | | |
| | | | | 1038 | | | |
| ETXM192 | ETXS384 | SEQ ID | ETXS383 | SEQ ID | 1 | 0.56 | HCII |
| | | NO: 679 | | NO: | | | |
| | | | | 1039 | | | |
| ETXM193 | ETXS386 | SEQ ID | ETXS385 | SEQ ID | 1.07 | 0.84 | HCII |
| | | NO: 680 | | NO: | | | |
| | | | | 1040 | | | |
| ETXM194 | ETXS388 | SEQ ID | ETXS387 | SEQ ID | 1.06 | 0.86 | HCII |
| | | NO: 681 | | NO: | | | |
| | | | | 1041 | | | |
| ETXM195 | ETXS390 | SEQ ID | ETXS389 | SEQ ID | 0.95 | 0.66 | HCII |
| | | NO: 682 | | NO: | | | |
| | | | | 1042 | | | |
| ETXM196 | ETXS392 | SEQ ID | ETXS391 | SEQ ID | 1.21 | 1.12 | HCII |
| | | NO: 683 | | NO: | | | |
| | | | | 1043 | | | |
| ETXM197 | ETXS394 | SEQ ID | ETXS393 | SEQ ID | 1.29 | 0.94 | HCII |
| | | NO: 684 | | NO: | | | |
| | | | | 1044 | | | |
| ETXM198 | ETXS396 | SEQ ID | ETXS395 | SEQ ID | 1.06 | 0.41 | HCII |
| | | NO: 685 | | NO: | | | |
| | | | | 1045 | | | |
| ETXM199 | ETXS398 | SEQ ID | ETXS397 | SEQ ID | 1.07 | 0.67 | HCII |
| | | NO: 686 | | NO: | | | |
| | | | | 1046 | | | |
| ETXM200 | ETXS400 | SEQ ID | ETXS399 | SEQ ID | 1.08 | 0.65 | HCII |
| | | NO: 687 | | NO: | | | |
| | | | | 1047 | | | |
| ETXM201 | ETXS402 | SEQ ID | ETXS401 | SEQ ID | 0.97 | 0.5 | HCII |
| | | NO: 688 | | NO: | | | |
| | | | | 1048 | | | |
| ETXM202 | ETXS404 | SEQ ID | ETXS403 | SEQ ID | 1.23 | 1.16 | HCII |
| | | NO: 689 | | NO: | | | |
| | | | | 1049 | | | |
| ETXM203 | ETXS406 | SEQ ID | ETXS405 | SEQ ID | 0.86 | 0.45 | HCII |
| | | NO: 690 | | NO: | | | |
| | | | | 1050 | | | |
| ETXM204 | ETXS408 | SEQ ID | ETXS407 | SEQ ID | 1.11 | 1.24 | HCII |
| | | NO: 691 | | NO: | | | |
| | | | | 1051 | | | |
| ETXM205 | ETXS410 | SEQ ID | ETXS409 | SEQ ID | 1.12 | 0.89 | HCII |
| | | NO: 692 | | NO: | | | |
| | | | | 1052 | | | |
| ETXM206 | ETXS412 | SEQ ID | ETXS411 | SEQ ID | 1.17 | 0.92 | HCII |
| | | NO: 693 | | NO: | | | |
| | | | | 1053 | | | |
| ETXM207 | ETXS414 | SEQ ID | ETXS413 | SEQ ID | 1.17 | 0.81 | HCII |
| | | NO: 694 | | NO: | | | |
| | | | | 1054 | | | |
| ETXM208 | ETXS416 | SEQ ID | ETXS415 | SEQ ID | 0.96 | 0.53 | HCII |
| | | NO: 695 | | NO: | | | |
| | | | | 1055 | | | |
| ETXM209 | ETXS418 | SEQ ID | ETXS417 | SEQ ID | 1.06 | 0.91 | HCII |
| | | NO: 696 | | NO: | | | |
| | | | | 1056 | | | |
| ETXM210 | ETXS420 | SEQ ID | ETXS419 | SEQ ID | 1.12 | 0.92 | HCII |
| | | NO: 697 | | NO: | | | |
| | | | | 1057 | | | |
| ETXM211 | ETXS422 | SEQ ID | ETXS421 | SEQ ID | 0.62 | 0.36 | HCII |
| | | NO: 698 | | NO: | | | |
| | | | | 1058 | | | |
| ETXM212 | ETXS424 | SEQ ID | ETXS423 | SEQ ID | 0.8 | 0.64 | HCII |
| | | NO: 699 | | NO: | | | |
| | | | | 1059 | | | |
| ETXM213 | ETXS426 | SEQ ID | ETXS425 | SEQ ID | 0.83 | 0.85 | HCII |
| | | NO: 700 | | NO: | | | |
| | | | | 1060 | | | |
| ETXM214 | ETXS428 | SEQ ID | ETXS427 | SEQ ID | 0.78 | 0.85 | HCII |
| | | NO: 701 | | NO: | | | |
| | | | | 1061 | | | |
| ETXM215 | ETXS430 | SEQ ID | ETXS429 | SEQ ID | 0.89 | 0.89 | HCII |
| | | NO: 702 | | NO: | | | |
| | | | | 1062 | | | |
| ETXM236 | ETXS472 | SEQ ID | ETXS471 | SEQ ID | 0.81 | 0.3 | HCII |
| | | NO: 703 | | NO: | | | |
| | | | | 1063 | | | |
| ETXM237 | ETXS474 | SEQ ID | ETXS473 | SEQ ID | 0.92 | 0.37 | HCII |
| | | NO: 704 | | NO: | | | |
| | | | | 1064 | | | |
| ETXM238 | ETXS476 | SEQ ID | ETXS475 | SEQ ID | 0.67 | 0.24 | HCII |
| | | NO: 705 | | NO: | | | |
| | | | | 1065 | | | |
| ETXM239 | ETXS478 | SEQ ID | ETXS477 | SEQ ID | 1.09 | 0.77 | HCII |
| | | NO: 706 | | NO: | | | |
| | | | | 1066 | | | |
| ETXM240 | ETXS480 | SEQ ID | ETXS479 | SEQ ID | 0.93 | 0.58 | HCII |
| | | NO: 707 | | NO: | | | |
| | | | | 1067 | | | |
| ETXM241 | ETXS482 | SEQ ID | ETXS481 | SEQ ID | 1.12 | 0.91 | HCII |
| | | NO: 708 | | NO: | | | |
| | | | | 1068 | | | |
| ETXM242 | ETXS484 | SEQ ID | ETXS483 | SEQ ID | 1.1 | 1.03 | HCII |
| | | NO: 709 | | NO: | | | |
| | | | | 1069 | | | |
| ETXM243 | ETXS486 | SEQ ID | ETXS485 | SEQ ID | 1.09 | 0.89 | HCII |
| | | NO: 710 | | NO: | | | |
| | | | | 1070 | | | |
| ETXM244 | ETXS488 | SEQ ID | ETXS487 | SEQ ID | 0.91 | 0.48 | HCII |
| | | NO: 711 | | NO: | | | |
| | | | | 1071 | | | |
| ETXM245 | ETXS490 | SEQ ID | ETXS489 | SEQ ID | 0.93 | 0.56 | HCII |
| | | NO: 712 | | NO: | | | |
| | | | | 1072 | | | |
| ETXM246 | ETXS492 | SEQ ID | ETXS491 | SEQ ID | 0.69 | 0.34 | HCII |
| | | NO: 713 | | NO: | | | |
| | | | | 1073 | | | |
| ETXM247 | ETXS494 | SEQ ID | ETXS493 | SEQ ID | 0.93 | 0.7 | HCII |
| | | NO: 714 | | NO: | | | |
| | | | | 1074 | | | |
| ETXM248 | ETXS496 | SEQ ID | ETXS495 | SEQ ID | 0.8 | 0.44 | HCII |
| | | NO: 715 | | NO: | | | |
| | | | | 1075 | | | |
| ETXM249 | ETXS498 | SEQ ID | ETXS497 | SEQ ID | 0.84 | 0.44 | HCII |
| | | NO: 716 | | NO: | | | |
| | | | | 1076 | | | |
| ETXM250 | ETXS500 | SEQ ID | ETXS499 | SEQ ID | 0.81 | 0.39 | HCII |
| | | NO: 717 | | NO: | | | |
| | | | | 1077 | | | |
| ETXM251 | ETXS502 | SEQ ID | ETXS501 | SEQ ID | 0.67 | 0.41 | HCII |
| | | NO: 718 | | NO: | | | |
| | | | | 1078 | | | |
| ETXM252 | ETXS504 | SEQ ID | ETXS503 | SEQ ID | 0.51 | 0.34 | HCII |
| | | NO: 719 | | NO: | | | |
| | | | | 1079 | | | |
| ETXM253 | ETXS506 | SEQ ID | ETXS505 | SEQ ID | 0.57 | 0.37 | HCII |
| | | NO: 720 | | NO: | | | |
| | | | | 1080 | | | |
| ETXM254 | ETXS508 | SEQ ID | ETXS507 | SEQ ID | 0.65 | 0.43 | HCII |
| | | NO: 721 | | NO: | | | |
| | | | | 1081 | | | |
| ETXM255 | ETXS510 | SEQ ID | ETXS509 | SEQ ID | 0.73 | 0.65 | HCII |
| | | NO: 722 | | NO: | | | |
| | | | | 1082 | | | |
| ETXM256 | ETXS512 | SEQ ID | ETXS511 | SEQ ID | 0.77 | 0.36 | HCII |
| | | NO: 723 | | NO: | | | |
| | | | | 1083 | | | |
| ETXM257 | ETXS514 | SEQ ID | ETXS513 | SEQ ID | 0.92 | 0.43 | HCII |
| | | NO: 724 | | NO: | | | |
| | | | | 1084 | | | |
| ETXM258 | ETXS516 | SEQ ID | ETXS515 | SEQ ID | 0.62 | 0.24 | HCII |
| | | NO: 725 | | NO: | | | |
| | | | | 1085 | | | |
| ETXM259 | ETXS518 | SEQ ID | ETXS517 | SEQ ID | 0.96 | 0.58 | HCII |
| | | NO: 726 | | NO: | | | |
| | | | | 1086 | | | |
| ETXM260 | ETXS520 | SEQ ID | ETXS519 | SEQ ID | 0.86 | 0.54 | HCII |
| | | NO: 727 | | NO: | | | |
| | | | | 1087 | | | |
| ETXM261 | ETXS522 | SEQ ID | ETXS521 | SEQ ID | 0.92 | 0.67 | HCII |
| | | NO: 728 | | NO: | | | |
| | | | | 1088 | | | |
| ETXM262 | ETXS524 | SEQ ID | ETXS523 | SEQ ID | 0.89 | 0.73 | HCII |
| | | NO: 729 | | NO: | | | |
| | | | | 1089 | | | |
| ETXM263 | ETXS526 | SEQ ID | ETXS525 | SEQ ID | 0.76 | 0.59 | HCII |
| | | NO: 730 | | NO: | | | |
| | | | | 1090 | | | |
| ETXM264 | ETXS528 | SEQ ID | ETXS527 | SEQ ID | 0.78 | 0.42 | HCII |
| | | NO: 731 | | NO: | | | |
| | | | | 1091 | | | |
| ETXM265 | ETXS530 | SEQ ID | ETXS529 | SEQ ID | 0.74 | 0.52 | HCII |
| | | NO: 732 | | NO: | | | |
| | | | | 1092 | | | |
| ETXM266 | ETXS532 | SEQ ID | ETXS531 | SEQ ID | 0.79 | 0.32 | HCII |
| | | NO: 733 | | NO: | | | |
| | | | | 1093 | | | |
| ETXM267 | ETXS534 | SEQ ID | ETXS533 | SEQ ID | 0.98 | 0.51 | HCII |
| | | NO: 734 | | NO: | | | |
| | | | | 1094 | | | |
| ETXM268 | ETXS536 | SEQ ID | ETXS535 | SEQ ID | 0.92 | 0.39 | HCII |
| | | NO: 735 | | NO: | | | |
| | | | | 1095 | | | |
| ETXM269 | ETXS538 | SEQ ID | ETXS537 | SEQ ID | 0.78 | 0.34 | HCII |
| | | NO: 736 | | NO: | | | |
| | | | | 1096 | | | |
| ETXM270 | ETXS540 | SEQ ID | ETXS539 | SEQ ID | 0.82 | 0.51 | HCII |
| | | NO: 737 | | NO: | | | |
| | | | | 1097 | | | |
| ETXM271 | ETXS542 | SEQ ID | ETXS541 | SEQ ID | 0.7 | 0.39 | HCII |
| | | NO: 738 | | NO: | | | |
| | | | | 1098 | | | |
| ETXM272 | ETXS544 | SEQ ID | ETXS543 | SEQ ID | 0.56 | 0.22 | HCII |
| | | NO: 739 | | NO: | | | |
| | | | | 1099 | | | |
| ETXM273 | ETXS546 | SEQ ID | ETXS545 | SEQ ID | 0.58 | 0.27 | HCII |
| | | NO: 740 | | NO: | | | |
| | | | | 1100 | | | |
| ETXM274 | ETXS548 | SEQ ID | ETXS547 | SEQ ID | 0.71 | 0.34 | HCII |
| | | NO: 741 | | NO: | | | |
| | | | | 1101 | | | |
| ETXM275 | ETXS550 | SEQ ID | ETXS549 | SEQ ID | 0.78 | 0.51 | HCII |
| | | NO: 742 | | NO: | | | |
| | | | | 1102 | | | |
| ETXM276 | ETXS552 | SEQ ID | ETXS551 | SEQ ID | 0.93 | 0.43 | HCII |
| | | NO: 743 | | NO: | | | |
| | | | | 1103 | | | |
| ETXM277 | ETXS554 | SEQ ID | ETXS553 | SEQ ID | 1.12 | 0.76 | HCII |
| | | NO: 744 | | NO: | | | |
| | | | | 1104 | | | |
| ETXM278 | ETXS556 | SEQ ID | ETXS555 | SEQ ID | 0.75 | 0.35 | HCII |
| | | NO: 745 | | NO: | | | |
| | | | | 1105 | | | |
| ETXM279 | ETXS558 | SEQ ID | ETXS557 | SEQ ID | 1.02 | 1.01 | HCII |
| | | NO: 746 | | NO: | | | |
| | | | | 1106 | | | |
| ETXM280 | ETXS560 | SEQ ID | ETXS559 | SEQ ID | 1.01 | 0.9 | HCII |
| | | NO: 747 | | NO: | | | |
| | | | | 1107 | | | |
| ETXM281 | ETXS562 | SEQ ID | ETXS561 | SEQ ID | 1.2 | 0.98 | HCII |
| | | NO: 748 | | NO: | | | |
| | | | | 1108 | | | |
| ETXM282 | ETXS564 | SEQ ID | ETXS563 | SEQ ID | 1.23 | 0.98 | HCII |
| | | NO: 749 | | NO: | | | |
| | | | | 1109 | | | |
| ETXM283 | ETXS566 | SEQ ID | ETXS565 | SEQ ID | 1.22 | 0.91 | HCII |
| | | NO: 750 | | NO: | | | |
| | | | | 1110 | | | |
| ETXM284 | ETXS568 | SEQ ID | ETXS567 | SEQ ID | 0.97 | 0.45 | HCII |
| | | NO: 751 | | NO: | | | |
| | | | | 1111 | | | |
| ETXM285 | ETXS570 | SEQ ID | ETXS569 | SEQ ID | 1.34 | 0.94 | HCII |
| | | NO: 752 | | NO: | | | |
| | | | | 1112 | | | |
| ETXM286 | ETXS572 | SEQ ID | ETXS571 | SEQ ID | 0.88 | 0.48 | HCII |
| | | NO: 753 | | NO: | | | |
| | | | | 1113 | | | |
| ETXM287 | ETXS574 | SEQ ID | ETXS573 | SEQ ID | 0.84 | 0.64 | HCII |
| | | NO: 754 | | NO: | | | |
| | | | | 1114 | | | |
| ETXM288 | ETXS576 | SEQ ID | ETXS575 | SEQ ID | 0.85 | 0.43 | HCII |
| | | NO: 755 | | NO: | | | |
| | | | | 1115 | | | |
| ETXM289 | ETXS578 | SEQ ID | ETXS577 | SEQ ID | 0.76 | 0.42 | HCII |
| | | NO: 756 | | NO: | | | |
| | | | | 1116 | | | |
| ETXM290 | ETXS580 | SEQ ID | ETXS579 | SEQ ID | 0.81 | 0.45 | HCII |
| | | NO: 757 | | NO: | | | |
| | | | | 1117 | | | |
| ETXM291 | ETXS582 | SEQ ID | ETXS581 | SEQ ID | 0.81 | 0.4 | HCII |
| | | NO: 758 | | NO: | | | |
| | | | | 1118 | | | |
| ETXM292 | ETXS584 | SEQ ID | ETXS583 | SEQ ID | 0.48 | 0.28 | HCII |
| | | NO: 759 | | NO: | | | |
| | | | | 1119 | | | |
| ETXM293 | ETXS586 | SEQ ID | ETXS585 | SEQ ID | 0.56 | 0.25 | HCII |
| | | NO: 760 | | NO: | | | |
| | | | | 1120 | | | |
| ETXM294 | ETXS588 | SEQ ID | ETXS587 | SEQ ID | 0.62 | 0.32 | HCII |
| | | NO: 761 | | NO: | | | |
| | | | | 1121 | | | |
| ETXM295 | ETXS590 | SEQ ID | ETXS589 | SEQ ID | 1 | 0.67 | HCII |
| | | NO: 762 | | NO: | | | |
| | | | | 1122 | | | |
| ETXM296 | ETXS592 | SEQ ID | ETXS591 | SEQ ID | 0.71 | 0.5 | HCII |
| | | NO: 763 | | NO: | | | |
| | | | | 1123 | | | |
| ETXM297 | ETXS594 | SEQ ID | ETXS593 | SEQ ID | 0.74 | 0.46 | HCII |
| | | NO: 764 | | NO: | | | |
| | | | | 1124 | | | |
| ETXM298 | ETXS596 | SEQ ID | ETXS595 | SEQ ID | 0.65 | 0.29 | HCII |
| | | NO: 765 | | NO: | | | |
| | | | | 1125 | | | |
| ETXM299 | ETXS598 | SEQ ID | ETXS597 | SEQ ID | 0.82 | 0.65 | HCII |
| | | NO: 766 | | NO: | | | |
| | | | | 1126 | | | |
| ETXM300 | ETXS600 | SEQ ID | ETXS599 | SEQ ID | 0.81 | 0.6 | HCII |
| | | NO: 767 | | NO: | | | |
| | | | | 1127 | | | |
| ETXM301 | ETXS602 | SEQ ID | ETXS601 | SEQ ID | 0.97 | 0.94 | HCII |
| | | NO: 768 | | NO: | | | |
| | | | | 1128 | | | |
| ETXM302 | ETXS604 | SEQ ID | ETXS603 | SEQ ID | 1.13 | 0.85 | HCII |
| | | NO: 769 | | NO: | | | |
| | | | | 1129 | | | |
| ETXM303 | ETXS606 | SEQ ID | ETXS605 | SEQ ID | 1.08 | 0.69 | HCII |
| | | NO: 770 | | NO: | | | |
| | | | | 1130 | | | |
| ETXM304 | ETXS608 | SEQ ID | ETXS607 | SEQ ID | 0.99 | 0.41 | HCII |
| | | NO: 771 | | NO: | | | |
| | | | | 1131 | | | |
| ETXM305 | ETXS610 | SEQ ID | ETXS609 | SEQ ID | 1.14 | 0.78 | HCII |
| | | NO: 772 | | NO: | | | |
| | | | | 1132 | | | |
| ETXM306 | ETXS612 | SEQ ID | ETXS611 | SEQ ID | 0.74 | 0.43 | HCII |
| | | NO: 773 | | NO: | | | |
| | | | | 1133 | | | |
| ETXM307 | ETXS614 | SEQ ID | ETXS613 | SEQ ID | 0.81 | 0.53 | HCII |
| | | NO: 774 | | NO: | | | |
| | | | | 1134 | | | |
| ETXM308 | ETXS616 | SEQ ID | ETXS615 | SEQ ID | 0.68 | 0.36 | HCII |
| | | NO: 775 | | NO: | | | |
| | | | | 1135 | | | |
| ETXM309 | ETXS618 | SEQ ID | ETXS617 | SEQ ID | 0.63 | 0.26 | HCII |
| | | NO: 776 | | NO: | | | |
| | | | | 1136 | | | |
| ETXM310 | ETXS620 | SEQ ID | ETXS619 | SEQ ID | 0.76 | 0.43 | HCII |
| | | NO: 777 | | NO: | | | |
| | | | | 1137 | | | |
| ETXM311 | ETXS622 | SEQ ID | ETXS621 | SEQ ID | 0.71 | 0.36 | HCII |
| | | NO: 778 | | NO: | | | |
| | | | | 1138 | | | |
| ETXM312 | ETXS624 | SEQ ID | ETXS623 | SEQ ID | 0.62 | 0.25 | HCII |
| | | NO: 779 | | NO: | | | |
| | | | | 1139 | | | |
| ETXM313 | ETXS626 | SEQ ID | ETXS625 | SEQ ID | 0.79 | 0.31 | HCII |
| | | NO: 780 | | NO: | | | |
| | | | | 1140 | | | |
| ETXM314 | ETXS628 | SEQ ID | ETXS627 | SEQ ID | 0.65 | 0.25 | HCII |
| | | NO: 781 | | NO: | | | |
| | | | | 1141 | | | |
| ETXM315 | ETXS630 | SEQ ID | ETXS629 | SEQ ID | 0.96 | 0.66 | HCII |
| | | NO: 782 | | NO: | | | |
| | | | | 1142 | | | |
| ETXM316 | ETXS632 | SEQ ID | ETXS631 | SEQ ID | 0.72 | 0.38 | ZPI |
| | | NO: 783 | | NO: | | | |
| | | | | 1143 | | | |
| ETXM317 | ETXS634 | SEQ ID | ETXS633 | SEQ ID | 1.01 | 0.54 | ZPI |
| | | NO: 784 | | NO: | | | |
| | | | | 1144 | | | |
| ETXM318 | ETXS636 | SEQ ID | ETXS635 | SEQ ID | 0.97 | 0.56 | ZPI |
| | | NO: 785 | | NO: | | | |
| | | | | 1145 | | | |
| ETXM319 | ETXS638 | SEQ ID | ETXS637 | SEQ ID | 0.91 | 0.43 | ZPI |
| | | NO: 786 | | NO: | | | |
| | | | | 1146 | | | |
| ETXM320 | ETXS640 | SEQ ID | ETXS639 | SEQ ID | 0.84 | 0.34 | ZPI |
| | | NO: 787 | | NO: | | | |
| | | | | 1147 | | | |
| ETXM321 | ETXS642 | SEQ ID | ETXS641 | SEQ ID | 0.58 | 0.27 | ZPI |
| | | NO: 788 | | NO: | | | |
| | | | | 1148 | | | |
| ETXM322 | ETXS644 | SEQ ID | ETXS643 | SEQ ID | 0.5 | 0.27 | ZPI |
| | | NO: 789 | | NO: | | | |
| | | | | 1149 | | | |
| ETXM323 | ETXS646 | SEQ ID | ETXS645 | SEQ ID | 0.69 | 0.3 | ZPI |
| | | NO: 790 | | NO: | | | |
| | | | | 1150 | | | |
| ETXM324 | ETXS648 | SEQ ID | ETXS647 | SEQ ID | 0.87 | 0.46 | ZPI |
| | | NO: 791 | | NO: | | | |
| | | | | 1151 | | | |
| ETXM325 | ETXS650 | SEQ ID | ETXS649 | SEQ ID | 0.74 | 0.31 | ZPI |
| | | NO: 792 | | NO: | | | |
| | | | | 1152 | | | |
| ETXM326 | ETXS652 | SEQ ID | ETXS651 | SEQ ID | 0.99 | 0.33 | ZPI |
| | | NO: 793 | | NO: | | | |
| | | | | 1153 | | | |
| ETXM327 | ETXS654 | SEQ ID | ETXS653 | SEQ ID | 0.79 | 0.37 | ZPI |
| | | NO: 794 | | NO: | | | |
| | | | | 1154 | | | |
| ETXM328 | ETXS656 | SEQ ID | ETXS655 | SEQ ID | 0.9 | 0.49 | ZPI |
| | | NO: 795 | | NO: | | | |
| | | | | 1155 | | | |
| ETXM329 | ETXS658 | SEQ ID | ETXS657 | SEQ ID | 1.11 | 0.81 | ZPI |
| | | NO: 796 | | NO: | | | |
| | | | | 1156 | | | |
| ETXM330 | ETXS660 | SEQ ID | ETXS659 | SEQ ID | 1 | 0.83 | ZPI |
| | | NO: 797 | | NO: | | | |
| | | | | 1157 | | | |
| ETXM331 | ETXS662 | SEQ ID | ETXS661 | SEQ ID | 1.04 | 0.84 | ZPI |
| | | NO: 798 | | NO: | | | |
| | | | | 1158 | | | |
| ETXM332 | ETXS664 | SEQ ID | ETXS663 | SEQ ID | 0.42 | 0.22 | ZPI |
| | | NO: 799 | | NO: | | | |
| | | | | 1159 | | | |
| ETXM333 | ETXS666 | SEQ ID | ETXS665 | SEQ ID | 0.58 | 0.28 | ZPI |
| | | NO: 800 | | NO: | | | |
| | | | | 1160 | | | |
| ETXM334 | ETXS668 | SEQ ID | ETXS667 | SEQ ID | 0.91 | 0.57 | ZPI |
| | | NO: 801 | | NO: | | | |
| | | | | 1161 | | | |
| ETXM335 | ETXS670 | SEQ ID | ETXS669 | SEQ ID | 1.04 | 0.74 | ZPI |
| | | NO: 802 | | NO: | | | |
| | | | | 1162 | | | |
| ETXM336 | ETXS672 | SEQ ID | ETXS671 | SEQ ID | 1.07 | 0.85 | ZPI |
| | | NO: 803 | | NO: | | | |
| | | | | 1163 | | | |
| ETXM337 | ETXS674 | SEQ ID | ETXS673 | SEQ ID | 0.84 | 0.51 | ZPI |
| | | NO: 804 | | NO: | | | |
| | | | | 1164 | | | |
| ETXM338 | ETXS676 | SEQ ID | ETXS675 | SEQ ID | 0.38 | 0.23 | ZPI |
| | | NO: 805 | | NO: | | | |
| | | | | 1165 | | | |
| ETXM339 | ETXS678 | SEQ ID | ETXS677 | SEQ ID | 0.85 | 0.4 | ZPI |
| | | NO: 806 | | NO: | | | |
| | | | | 1166 | | | |
| ETXM340 | ETXS680 | SEQ ID | ETXS679 | SEQ ID | 0.75 | 0.36 | ZPI |
| | | NO: 807 | | NO: | | | |
| | | | | 1167 | | | |
| ETXM341 | ETXS682 | SEQ ID | ETXS681 | SEQ ID | 0.55 | 0.22 | ZPI |
| | | NO: 808 | | NO: | | | |
| | | | | 1168 | | | |
| ETXM342 | ETXS684 | SEQ ID | ETXS683 | SEQ ID | 0.55 | 0.42 | ZPI |
| | | NO: 809 | | NO: | | | |
| | | | | 1169 | | | |
| ETXM343 | ETXS686 | SEQ ID | ETXS685 | SEQ ID | 0.45 | 0.29 | ZPI |
| | | NO: 810 | | NO: | | | |
| | | | | 1170 | | | |
| ETXM344 | ETXS688 | SEQ ID | ETXS687 | SEQ ID | 0.98 | 1.01 | ZPI |
| | | NO: 811 | | NO: | | | |
| | | | | 1171 | | | |
| ETXM345 | ETXS690 | SEQ ID | ETXS689 | SEQ ID | 0.78 | 0.57 | ZPI |
| | | NO: 812 | | NO: | | | |
| | | | | 1172 | | | |
| ETXM346 | ETXS692 | SEQ ID | ETXS691 | SEQ ID | 1.09 | 1.12 | ZPI |
| | | NO: 813 | | NO: | | | |
| | | | | 1173 | | | |
| ETXM347 | ETXS694 | SEQ ID | ETXS693 | SEQ ID | 0.93 | 0.45 | ZPI |
| | | NO: 814 | | NO: | | | |
| | | | | 1174 | | | |
| ETXM348 | ETXS696 | SEQ ID | ETXS695 | SEQ ID | 0.91 | 0.65 | ZPI |
| | | NO: 815 | | NO: | | | |
| | | | | 1175 | | | |
| ETXM349 | ETXS698 | SEQ ID | ETXS697 | SEQ ID | 0.88 | 0.49 | ZPI |
| | | NO: 816 | | NO: | | | |
| | | | | 1176 | | | |
| ETXM350 | ETXS700 | SEQ ID | ETXS699 | SEQ ID | 0.87 | 0.75 | ZPI |
| | | NO: 817 | | NO: | | | |
| | | | | 1177 | | | |
| ETXM351 | ETXS702 | SEQ ID | ETXS701 | SEQ ID | 0.96 | 0.96 | ZPI |
| | | NO: 818 | | NO: | | | |
| | | | | 1178 | | | |
| ETXM352 | ETXS704 | SEQ ID | ETXS703 | SEQ ID | 0.95 | 1.04 | ZPI |
| | | NO: 819 | | NO: | | | |
| | | | | 1179 | | | |
| ETXM353 | ETXS706 | SEQ ID | ETXS705 | SEQ ID | 0.71 | 0.5 | ZPI |
| | | NO: 820 | | NO: | | | |
| | | | | 1180 | | | |
| ETXM354 | ETXS708 | SEQ ID | ETXS707 | SEQ ID | 0.7 | 0.43 | ZPI |
| | | NO: 821 | | NO: | | | |
| | | | | 1181 | | | |
| ETXM355 | ETXS710 | SEQ ID | ETXS709 | SEQ ID | 0.69 | 0.34 | ZPI |
| | | NO: 822 | | NO: | | | |
| | | | | 1182 | | | |
| ETXM356 | ETXS712 | SEQ ID | ETXS711 | SEQ ID | 0.92 | 0.71 | ZPI |
| | | NO: 823 | | NO: | | | |
| | | | | 1183 | | | |
| ETXM357 | ETXS714 | SEQ ID | ETXS713 | SEQ ID | 0.88 | 0.49 | ZPI |
| | | NO: 824 | | NO: | | | |
| | | | | 1184 | | | |
| ETXM358 | ETXS716 | SEQ ID | ETXS715 | SEQ ID | 0.98 | 0.5 | ZPI |
| | | NO: 825 | | NO: | | | |
| | | | | 1185 | | | |
| ETXM359 | ETXS718 | SEQ ID | ETXS717 | SEQ ID | 0.66 | 0.33 | ZPI |
| | | NO: 826 | | NO: | | | |
| | | | | 1186 | | | |
| ETXM360 | ETXS720 | SEQ ID | ETXS719 | SEQ ID | 0.75 | 0.54 | ZPI |
| | | NO: 827 | | NO: | | | |
| | | | | 1187 | | | |
| ETXM361 | ETXS722 | SEQ ID | ETXS721 | SEQ ID | 0.68 | 0.48 | ZPI |
| | | NO: 828 | | NO: | | | |
| | | | | 1188 | | | |
| ETXM362 | ETXS724 | SEQ ID | ETXS723 | SEQ ID | 0.95 | 0.9 | ZPI |
| | | NO: 829 | | NO: | | | |
| | | | | 1189 | | | |
| ETXM363 | ETXS726 | SEQ ID | ETXS725 | SEQ ID | 0.96 | 0.75 | ZPI |
| | | NO: 830 | | NO: | | | |
| | | | | 1190 | | | |
| ETXM364 | ETXS728 | SEQ ID | ETXS727 | SEQ ID | 0.88 | 0.44 | ZPI |
| | | NO: 831 | | NO: | | | |
| | | | | 1191 | | | |
| ETXM365 | ETXS730 | SEQ ID | ETXS729 | SEQ ID | 0.82 | 0.49 | ZPI |
| | | NO: 832 | | NO: | | | |
| | | | | 1192 | | | |
| ETXM366 | ETXS732 | SEQ ID | ETXS731 | SEQ ID | 0.95 | 0.6 | ZPI |
| | | NO: 833 | | NO: | | | |
| | | | | 1193 | | | |
| ETXM367 | ETXS734 | SEQ ID | ETXS733 | SEQ ID | 0.92 | 0.51 | ZPI |
| | | NO: 834 | | NO: | | | |
| | | | | 1194 | | | |
| ETXM368 | ETXS736 | SEQ ID | ETXS735 | SEQ ID | 1.02 | 0.84 | ZPI |
| | | NO: 835 | | NO: | | | |
| | | | | 1195 | | | |
| ETXM369 | ETXS738 | SEQ ID | ETXS737 | SEQ ID | 1.02 | 1 | ZPI |
| | | NO: 836 | | NO: | | | |
| | | | | 1196 | | | |
| ETXM370 | ETXS740 | SEQ ID | ETXS739 | SEQ ID | 1.37 | 0.96 | ZPI |
| | | NO: 837 | | NO: | | | |
| | | | | 1197 | | | |
| ETXM371 | ETXS742 | SEQ ID | ETXS741 | SEQ ID | 0.94 | 0.65 | ZPI |
| | | NO: 838 | | NO: | | | |
| | | | | 1198 | | | |
| ETXM372 | ETXS744 | SEQ ID | ETXS743 | SEQ ID | 0.95 | 0.67 | ZPI |
| | | NO: 839 | | NO: | | | |
| | | | | 1199 | | | |
| ETXM373 | ETXS746 | SEQ ID | ETXS745 | SEQ ID | 1.05 | 0.96 | ZPI |
| | | NO: 840 | | NO: | | | |
| | | | | 1200 | | | |
| ETXM374 | ETXS748 | SEQ ID | ETXS747 | SEQ ID | 0.97 | 0.91 | ZPI |
| | | NO: 841 | | NO: | | | |
| | | | | 1201 | | | |
| ETXM375 | ETXS750 | SEQ ID | ETXS749 | SEQ ID | 0.81 | 0.39 | ZPI |
| | | NO: 842 | | NO: | | | |
| | | | | 1202 | | | |
| ETXM376 | ETXS752 | SEQ ID | ETXS751 | SEQ ID | 0.97 | 0.76 | ZPI |
| | | NO: 843 | | NO: | | | |
| | | | | 1203 | | | |
| ETXM377 | ETXS754 | SEQ ID | ETXS753 | SEQ ID | 0.93 | 0.59 | ZPI |
| | | NO: 844 | | NO: | | | |
| | | | | 1204 | | | |
| ETXM378 | ETXS756 | SEQ ID | ETXS755 | SEQ ID | 0.93 | 0.52 | ZPI |
| | | NO: 845 | | NO: | | | |
| | | | | 1205 | | | |
| ETXM379 | ETXS758 | SEQ ID | ETXS757 | SEQ ID | 0.96 | 0.81 | ZPI |
| | | NO: 846 | | NO: | | | |
| | | | | 1206 | | | |
| ETXM380 | ETXS760 | SEQ ID | ETXS759 | SEQ ID | 0.61 | 0.31 | ZPI |
| | | NO: 847 | | NO: | | | |
| | | | | 1207 | | | |
| ETXM381 | ETXS762 | SEQ ID | ETXS761 | SEQ ID | 0.84 | 0.82 | ZPI |
| | | NO: 848 | | NO: | | | |
| | | | | 1208 | | | |
| ETXM382 | ETXS764 | SEQ ID | ETXS763 | SEQ ID | 0.8 | 0.47 | ZPI |
| | | NO: 849 | | NO: | | | |
| | | | | 1209 | | | |
| ETXM383 | ETXS766 | SEQ ID | ETXS765 | SEQ ID | 0.82 | 0.37 | ZPI |
| | | NO: 850 | | NO: | | | |
| | | | | 1210 | | | |
| ETXM384 | ETXS768 | SEQ ID | ETXS767 | SEQ ID | 0.67 | 0.38 | ZPI |
| | | NO: 851 | | NO: | | | |
| | | | | 1211 | | | |
| ETXM385 | ETXS770 | SEQ ID | ETXS769 | SEQ ID | 0.9 | 0.87 | ZPI |
| | | NO: 852 | | NO: | | | |
| | | | | 1212 | | | |
| ETXM386 | ETXS772 | SEQ ID | ETXS771 | SEQ ID | 0.91 | 0.73 | ZPI |
| | | NO: 853 | | NO: | | | |
| | | | | 1213 | | | |
| ETXM387 | ETXS774 | SEQ ID | ETXS773 | SEQ ID | 0.86 | 0.97 | ZPI |
| | | NO: 854 | | NO: | | | |
| | | | | 1214 | | | |
| ETXM388 | ETXS776 | SEQ ID | ETXS775 | SEQ ID | 0.96 | 0.7 | ZPI |
| | | NO: 855 | | NO: | | | |
| | | | | 1215 | | | |
| ETXM389 | ETXS778 | SEQ ID | ETXS777 | SEQ ID | 0.95 | 0.68 | ZPI |
| | | NO: 856 | | NO: | | | |
| | | | | 1216 | | | |
| ETXM390 | ETXS780 | SEQ ID | ETXS779 | SEQ ID | 0.87 | 0.51 | ZPI |
| | | NO: 857 | | NO: | | | |
| | | | | 1217 | | | |
| ETXM391 | ETXS782 | SEQ ID | ETXS781 | SEQ ID | 0.76 | 0.35 | ZPI |
| | | NO: 858 | | NO: | | | |
| | | | | 1218 | | | |
| ETXM392 | ETXS784 | SEQ ID | ETXS783 | SEQ ID | 0.99 | 0.76 | ZPI |
| | | NO: 859 | | NO: | | | |
| | | | | 1219 | | | |
| ETXM393 | ETXS786 | SEQ ID | ETXS785 | SEQ ID | 0.94 | 1.06 | ZPI |
| | | NO: 860 | | NO: | | | |
| | | | | 1220 | | | |
| ETXM394 | ETXS788 | SEQ ID | ETXS787 | SEQ ID | 0.85 | 0.8 | ZPI |
| | | NO: 861 | | NO: | | | |
| | | | | 1221 | | | |
| ETXM395 | ETXS790 | SEQ ID | ETXS789 | SEQ ID | 0.95 | 0.53 | ZPI |
| | | NO: 862 | | NO: | | | |
| | | | | 1222 | | | |
| ETXM396 | ETXS792 | SEQ ID | ETXS791 | SEQ ID | 0.62 | 0.27 | ZPI |
| | | NO: 863 | | NO: | | | |
| | | | | 1223 | | | |
| ETXM397 | ETXS794 | SEQ ID | ETXS793 | SEQ ID | 0.96 | 0.64 | ZPI |
| | | NO: 864 | | NO: | | | |
| | | | | 1224 | | | |
| ETXM398 | ETXS796 | SEQ ID | ETXS795 | SEQ ID | 0.93 | 0.55 | ZPI |
| | | NO: 865 | | NO: | | | |
| | | | | 1225 | | | |
| ETXM399 | ETXS798 | SEQ ID | ETXS797 | SEQ ID | 0.94 | 0.66 | ZPI |
| | | NO: 866 | | NO: | | | |
| | | | | 1226 | | | |
| ETXM400 | ETXS800 | SEQ ID | ETXS799 | SEQ ID | 0.77 | 0.57 | ZPI |
| | | NO: 867 | | NO: | | | |
| | | | | 1227 | | | |
| ETXM401 | ETXS802 | SEQ ID | ETXS801 | SEQ ID | 0.69 | 0.25 | ZPI |
| | | NO: 868 | | NO: | | | |
| | | | | 1228 | | | |
| ETXM402 | ETXS804 | SEQ ID | ETXS803 | SEQ ID | 1.05 | 0.95 | ZPI |
| | | NO: 869 | | NO: | | | |
| | | | | 1229 | | | |
| ETXM403 | ETXS806 | SEQ ID | ETXS805 | SEQ ID | 0.86 | 0.5 | ZPI |
| | | NO: 870 | | NO: | | | |
| | | | | 1230 | | | |
| ETXM404 | ETXS808 | SEQ ID | ETXS807 | SEQ ID | 0.83 | 0.35 | ZPI |
| | | NO: 871 | | NO: | | | |
| | | | | 1231 | | | |
| ETXM405 | ETXS810 | SEQ ID | ETXS809 | SEQ ID | 0.97 | 0.73 | ZPI |
| | | NO: 872 | | NO: | | | |
| | | | | 1232 | | | |
| ETXM406 | ETXS812 | SEQ ID | ETXS811 | SEQ ID | 0.84 | 0.33 | ZPI |
| | | NO: 873 | | NO: | | | |
| | | | | 1233 | | | |
| ETXM407 | ETXS814 | SEQ ID | ETXS813 | SEQ ID | 0.77 | 0.51 | ZPI |
| | | NO: 874 | | NO: | | | |
| | | | | 1234 | | | |
| ETXM408 | ETXS816 | SEQ ID | ETXS815 | SEQ ID | 0.89 | 0.51 | ZPI |
| | | NO: 875 | | NO: | | | |
| | | | | 1235 | | | |
| ETXM409 | ETXS818 | SEQ ID | ETXS817 | SEQ ID | 1 | 0.59 | ZPI |
| | | NO: 876 | | NO: | | | |
| | | | | 1236 | | | |
| ETXM410 | ETXS820 | SEQ ID | ETXS819 | SEQ ID | 0.98 | 0.71 | ZPI |
| | | NO: 877 | | NO: | | | |
| | | | | 1237 | | | |
| ETXM411 | ETXS822 | SEQ ID | ETXS821 | SEQ ID | 0.77 | 0.36 | ZPI |
| | | NO: 878 | | NO: | | | |
| | | | | 1238 | | | |
| ETXM412 | ETXS824 | SEQ ID | ETXS823 | SEQ ID | 0.97 | 0.42 | ZPI |
| | | NO: 879 | | NO: | | | |
| | | | | 1239 | | | |
| ETXM413 | ETXS826 | SEQ ID | ETXS825 | SEQ ID | 1 | 0.77 | ZPI |
| | | NO: 880 | | NO: | | | |
| | | | | 1240 | | | |
| ETXM414 | ETXS828 | SEQ ID | ETXS827 | SEQ ID | 0.98 | 0.79 | ZPI |
| | | NO: 881 | | NO: | | | |
| | | | | 1241 | | | |
| ETXM415 | ETXS830 | SEQ ID | ETXS829 | SEQ ID | 0.96 | 0.62 | ZPI |
| | | NO: 882 | | NO: | | | |
| | | | | 1242 | | | |
| ETXM436 | ETXS872 | SEQ ID | ETXS871 | SEQ ID | 0.72 | 0.44 | ZPI |
| | | NO: 883 | | NO: | | | |
| | | | | 1243 | | | |
| ETXM437 | ETXS874 | SEQ ID | ETXS873 | SEQ ID | 0.98 | 0.47 | ZPI |
| | | NO: 884 | | NO: | | | |
| | | | | 1244 | | | |
| ETXM438 | ETXS876 | SEQ ID | ETXS875 | SEQ ID | 1.05 | 0.75 | ZPI |
| | | NO: 885 | | NO: | | | |
| | | | | 1245 | | | |
| ETXM439 | ETXS878 | SEQ ID | ETXS877 | SEQ ID | 0.91 | 0.49 | ZPI |
| | | NO: 886 | | NO: | | | |
| | | | | 1246 | | | |
| ETXM440 | ETXS880 | SEQ ID | ETXS879 | SEQ ID | 0.91 | 0.46 | ZPI |
| | | NO: 887 | | NO: | | | |
| | | | | 1247 | | | |
| ETXM441 | ETXS882 | SEQ ID | ETXS881 | SEQ ID | 0.74 | 0.36 | ZPI |
| | | NO: 888 | | NO: | | | |
| | | | | 1248 | | | |
| ETXM442 | ETXS884 | SEQ ID | ETXS883 | SEQ ID | 0.62 | 0.36 | ZPI |
| | | NO: 889 | | NO: | | | |
| | | | | 1249 | | | |
| ETXM443 | ETXS886 | SEQ ID | ETXS885 | SEQ ID | 0.73 | 0.3 | ZPI |
| | | NO: 890 | | NO: | | | |
| | | | | 1250 | | | |
| ETXM444 | ETXS888 | SEQ ID | ETXS887 | SEQ ID | 1 | 0.59 | ZPI |
| | | NO: 891 | | NO: | | | |
| | | | | 1251 | | | |
| ETXM445 | ETXS890 | SEQ ID | ETXS889 | SEQ ID | 0.71 | 0.37 | ZPI |
| | | NO: 892 | | NO: | | | |
| | | | | 1252 | | | |
| ETXM446 | ETXS892 | SEQ ID | ETXS891 | SEQ ID | 0.73 | 0.27 | ZPI |
| | | NO: 893 | | NO: | | | |
| | | | | 1253 | | | |
| ETXM447 | ETXS894 | SEQ ID | ETXS893 | SEQ ID | 0.81 | 0.39 | ZPI |
| | | NO: 894 | | NO: | | | |
| | | | | 1254 | | | |
| ETXM448 | ETXS896 | SEQ ID | ETXS895 | SEQ ID | 0.81 | 0.61 | ZPI |
| | | NO: 895 | | NO: | | | |
| | | | | 1255 | | | |
| ETXM449 | ETXS898 | SEQ ID | ETXS897 | SEQ ID | 0.91 | 0.8 | ZPI |
| | | NO: 896 | | NO: | | | |
| | | | | 1256 | | | |
| ETXM450 | ETXS900 | SEQ ID | ETXS899 | SEQ ID | 0.97 | 0.52 | ZPI |
| | | NO: 897 | | NO: | | | |
| | | | | 1257 | | | |
| ETXM451 | ETXS902 | SEQ ID | ETXS901 | SEQ ID | 0.96 | 0.61 | ZPI |
| | | NO: 898 | | NO: | | | |
| | | | | 1258 | | | |
| ETXM452 | ETXS904 | SEQ ID | ETXS903 | SEQ ID | 0.4 | 0.24 | ZPI |
| | | NO: 899 | | NO: | | | |
| | | | | 1259 | | | |
| ETXM453 | ETXS906 | SEQ ID | ETXS905 | SEQ ID | 0.62 | 0.3 | ZPI |
| | | NO: 900 | | NO: | | | |
| | | | | 1260 | | | |
| ETXM454 | ETXS908 | SEQ ID | ETXS907 | SEQ ID | 0.81 | 0.46 | ZPI |
| | | NO: 901 | | NO: | | | |
| | | | | 1261 | | | |
| ETXM455 | ETXS910 | SEQ ID | ETXS909 | SEQ ID | 0.94 | 0.68 | ZPI |
| | | NO: 902 | | NO: | | | |
| | | | | 1262 | | | |
| ETXM456 | ETXS912 | SEQ ID | ETXS911 | SEQ ID | 0.75 | 0.36 | ZPI |
| | | NO: 903 | | NO: | | | |
| | | | | 1263 | | | |
| ETXM457 | ETXS914 | SEQ ID | ETXS913 | SEQ ID | 0.98 | 0.52 | ZPI |
| | | NO: 904 | | NO: | | | |
| | | | | 1264 | | | |
| ETXM458 | ETXS916 | SEQ ID | ETXS915 | SEQ ID | 1 | 0.61 | ZPI |
| | | NO: 905 | | NO: | | | |
| | | | | 1265 | | | |
| ETXM459 | ETXS918 | SEQ ID | ETXS917 | SEQ ID | 0.92 | 0.44 | ZPI |
| | | NO: 906 | | NO: | | | |
| | | | | 1266 | | | |
| ETXM460 | ETXS920 | SEQ ID | ETXS919 | SEQ ID | 0.86 | 0.4 | ZPI |
| | | NO: 907 | | NO: | | | |
| | | | | 1267 | | | |
| ETXM461 | ETXS922 | SEQ ID | ETXS921 | SEQ ID | 0.84 | 0.27 | ZPI |
| | | NO: 908 | | NO: | | | |
| | | | | 1268 | | | |
| ETXM462 | ETXS924 | SEQ ID | ETXS923 | SEQ ID | 0.72 | 0.33 | ZPI |
| | | NO: 909 | | NO: | | | |
| | | | | 1269 | | | |
| ETXM463 | ETXS926 | SEQ ID | ETXS925 | SEQ ID | 0.76 | 0.35 | ZPI |
| | | NO: 910 | | NO: | | | |
| | | | | 1270 | | | |
| ETXM464 | ETXS928 | SEQ ID | ETXS927 | SEQ ID | 0.95 | 0.55 | ZPI |
| | | NO: 911 | | NO: | | | |
| | | | | 1271 | | | |
| ETXM465 | ETXS930 | SEQ ID | ETXS929 | SEQ ID | 0.77 | 0.36 | ZPI |
| | | NO: 912 | | NO: | | | |
| | | | | 1272 | | | |
| ETXM466 | ETXS932 | SEQ ID | ETXS931 | SEQ ID | 0.84 | 0.33 | ZPI |
| | | NO: 913 | | NO: | | | |
| | | | | 1273 | | | |
| ETXM467 | ETXS934 | SEQ ID | ETXS933 | SEQ ID | 0.91 | 0.39 | ZPI |
| | | NO: 914 | | NO: | | | |
| | | | | 1274 | | | |
| ETXM468 | ETXS936 | SEQ ID | ETXS935 | SEQ ID | 1.14 | 0.8 | ZPI |
| | | NO: 915 | | NO: | | | |
| | | | | 1275 | | | |
| ETXM469 | ETXS938 | SEQ ID | ETXS937 | SEQ ID | 1.17 | 0.67 | ZPI |
| | | NO: 916 | | NO: | | | |
| | | | | 1276 | | | |
| ETXM470 | ETXS940 | SEQ ID | ETXS939 | SEQ ID | 1.12 | 0.79 | ZPI |
| | | NO: 917 | | NO: | | | |
| | | | | 1277 | | | |
| ETXM471 | ETXS942 | SEQ ID | ETXS941 | SEQ ID | 1.16 | 0.86 | ZPI |
| | | NO: 918 | | NO: | | | |
| | | | | 1278 | | | |
| ETXM472 | ETXS944 | SEQ ID | ETXS943 | SEQ ID | 0.49 | 0.25 | ZPI |
| | | NO: 919 | | NO: | | | |
| | | | | 1279 | | | |
| ETXM473 | ETXS946 | SEQ ID | ETXS945 | SEQ ID | 0.91 | 0.34 | ZPI |
| | | NO: 920 | | NO: | | | |
| | | | | 1280 | | | |
| ETXM474 | ETXS948 | SEQ ID | ETXS947 | SEQ ID | 1.12 | 0.68 | ZPI |
| | | NO: 921 | | NO: | | | |
| | | | | 1281 | | | |
| ETXM475 | ETXS950 | SEQ ID | ETXS949 | SEQ ID | 1.25 | 0.84 | ZPI |
| | | NO: 922 | | NO: | | | |
| | | | | 1282 | | | |
| ETXM476 | ETXS952 | SEQ ID | ETXS951 | SEQ ID | 0.87 | 0.42 | ZPI |
| | | NO: 923 | | NO: | | | |
| | | | | 1283 | | | |
| ETXM477 | ETXS954 | SEQ ID | ETXS953 | SEQ ID | 1.12 | 0.52 | ZPI |
| | | NO: 924 | | NO: | | | |
| | | | | 1284 | | | |
| ETXM478 | ETXS956 | SEQ ID | ETXS955 | SEQ ID | 1.03 | 0.62 | ZPI |
| | | NO: 925 | | NO: | | | |
| | | | | 1285 | | | |
| ETXM479 | ETXS958 | SEQ ID | ETXS957 | SEQ ID | 1.13 | 0.51 | ZPI |
| | | NO: 926 | | NO: | | | |
| | | | | 1286 | | | |
| ETXM480 | ETXS960 | SEQ ID | ETXS959 | SEQ ID | 0.93 | 0.56 | ZPI |
| | | NO: 927 | | NO: | | | |
| | | | | 1287 | | | |
| ETXM481 | ETXS962 | SEQ ID | ETXS961 | SEQ ID | 0.89 | 0.36 | ZPI |
| | | NO: 928 | | NO: | | | |
| | | | | 1288 | | | |
| ETXM482 | ETXS964 | SEQ ID | ETXS963 | SEQ ID | 0.68 | 0.46 | ZPI |
| | | NO: 929 | | NO: | | | |
| | | | | 1289 | | | |
| ETXM483 | ETXS966 | SEQ ID | ETXS965 | SEQ ID | 0.82 | 0.5 | ZPI |
| | | NO: 930 | | NO: | | | |
| | | | | 1290 | | | |
| ETXM484 | ETXS968 | SEQ ID | ETXS967 | SEQ ID | 1.06 | 0.74 | ZPI |
| | | NO: 931 | | NO: | | | |
| | | | | 1291 | | | |
| ETXM485 | ETXS970 | SEQ ID | ETXS969 | SEQ ID | 0.91 | 0.41 | ZPI |
| | | NO: 932 | | NO: | | | |
| | | | | 1292 | | | |
| ETXM486 | ETXS972 | SEQ ID | ETXS971 | SEQ ID | 0.68 | 0.23 | ZPI |
| | | NO: 933 | | NO: | | | |
| | | | | 1293 | | | |
| ETXM487 | ETXS974 | SEQ ID | ETXS973 | SEQ ID | 0.8 | 0.31 | ZPI |
| | | NO: 934 | | NO: | | | |
| | | | | 1294 | | | |
| ETXM488 | ETXS976 | SEQ ID | ETXS975 | SEQ ID | 0.89 | 0.64 | ZPI |
| | | NO: 935 | | NO: | | | |
| | | | | 1295 | | | |
| ETXM489 | ETXS978 | SEQ ID | ETXS977 | SEQ ID | 0.89 | 0.67 | ZPI |
| | | NO: 936 | | NO: | | | |
| | | | | 1296 | | | |
| ETXM490 | ETXS980 | SEQ ID | ETXS979 | SEQ ID | 0.91 | 0.56 | ZPI |
| | | NO: 937 | | NO: | | | |
| | | | | 1297 | | | |
| ETXM491 | ETXS982 | SEQ ID | ETXS981 | SEQ ID | 1.06 | 0.75 | ZPI |
| | | NO: 938 | | NO: | | | |
| | | | | 1298 | | | |
| ETXM492 | ETXS984 | SEQ ID | ETXS983 | SEQ ID | 0.43 | 0.22 | ZPI |
| | | NO: 939 | | NO: | | | |
| | | | | 1299 | | | |
| ETXM493 | ETXS986 | SEQ ID | ETXS985 | SEQ ID | 0.59 | 0.33 | ZPI |
| | | NO: 940 | | NO: | | | |
| | | | | 1300 | | | |
| ETXM494 | ETXS988 | SEQ ID | ETXS987 | SEQ ID | 0.93 | 0.59 | ZPI |
| | | NO: 941 | | NO: | | | |
| | | | | 1301 | | | |
| ETXM495 | ETXS990 | SEQ ID | ETXS989 | SEQ ID | 1.08 | 0.74 | ZPI |
| | | NO: 942 | | NO: | | | |
| | | | | 1302 | | | |
| ETXM496 | ETXS992 | SEQ ID | ETXS991 | SEQ ID | 0.73 | 0.42 | ZPI |
| | | NO: 943 | | NO: | | | |
| | | | | 1303 | | | |
| ETXM497 | ETXS994 | SEQ ID | ETXS993 | SEQ ID | 1.01 | 0.59 | ZPI |
| | | NO: 944 | | NO: | | | |
| | | | | 1304 | | | |
| ETXM498 | ETXS996 | SEQ ID | ETXS995 | SEQ ID | 0.95 | 0.59 | ZPI |
| | | NO: 945 | | NO: | | | |
| | | | | 1305 | | | |
| ETXM499 | ETXS998 | SEQ ID | ETXS997 | SEQ ID | 1.08 | 0.53 | ZPI |
| | | NO: 946 | | NO: | | | |
| | | | | 1306 | | | |
| ETXM500 | ETXS100 | SEQ ID | ETXS999 | SEQ ID | 0.87 | 0.46 | ZPI |
| | 0 | NO: 947 | | NO: | | | |
| | | | | 1307 | | | |
| ETXM501 | ETXS100 | SEQ ID | ETXS100 | SEQ ID | 0.6 | 0.27 | ZPI |
| | 2 | NO: 948 | 1 | NO: | | | |
| | | | | 1308 | | | |
| ETXM502 | ETXS100 | SEQ ID | ETXS100 | SEQ ID | 0.6 | 0.28 | ZPI |
| | 4 | NO: 949 | 3 | NO: | | | |
| | | | | 1309 | | | |
| ETXM503 | ETXS100 | SEQ ID | ETXS100 | SEQ ID | 0.73 | 0.36 | ZPI |
| | 6 | NO: 950 | 5 | NO: | | | |
| | | | | 1310 | | | |
| ETXM504 | ETXS100 | SEQ ID | ETXS100 | SEQ ID | 0.9 | 0.68 | ZPI |
| | 8 | NO: 951 | 7 | NO: | | | |
| | | | | 1311 | | | |
| ETXM505 | ETXS101 | SEQ ID | ETXS100 | SEQ ID | 0.72 | 0.36 | ZPI |
| | 0 | NO: 952 | 9 | NO: | | | |
| | | | | 1312 | | | |
| ETXM506 | ETXS101 | SEQ ID | ETXS101 | SEQ ID | 0.62 | 0.25 | ZPI |
| | 2 | NO: 953 | 1 | NO: | | | |
| | | | | 1313 | | | |
| ETXM507 | ETXS101 | SEQ ID | ETXS101 | SEQ ID | 1.21 | 0.33 | ZPI |
| | 4 | NO: 954 | 3 | NO: | | | |
| | | | | 1314 | | | |
| ETXM508 | ETXS101 | SEQ ID | ETXS101 | SEQ ID | 1.08 | 0.83 | ZPI |
| | 6 | NO: 955 | 5 | NO: | | | |
| | | | | 1315 | | | |
| ETXM509 | ETXS101 | SEQ ID | ETXS101 | SEQ ID | 1.09 | 0.85 | ZPI |
| | 8 | NO: 956 | 7 | NO: | | | |
| | | | | 1316 | | | |
| ETXM510 | ETXS102 | SEQ ID | ETXS101 | SEQ ID | 0.98 | 0.62 | ZPI |
| | 0 | NO: 957 | 9 | NO: | | | |
| | | | | 1317 | | | |
| ETXM511 | ETXS102 | SEQ ID | ETXS102 | SEQ ID | 0.81 | 0.87 | ZPI |
| | 2 | NO: 958 | 1 | NO: | | | |
| | | | | 1318 | | | |
| ETXM512 | ETXS102 | SEQ ID | ETXS102 | SEQ ID | 0.34 | 0.17 | ZPI |
| | 4 | NO: 959 | 3 | NO: | | | |
| | | | | 1319 | | | |
| ETXM513 | ETXS102 | SEQ ID | ETXS102 | SEQ ID | 0.49 | 0.22 | ZPI |
| | 6 | NO: 960 | 5 | NO: | | | |
| | | | | 1320 | | | |
| ETXM514 | ETXS102 | SEQ ID | ETXS102 | SEQ ID | 0.84 | 0.56 | ZPI |
| | 8 | NO: 961 | 7 | NO: | | | |
| | | | | 1321 | | | |
| ETXM515 | ETXS103 | SEQ ID | ETXS102 | SEQ ID | 0.93 | 0.74 | ZPI |
| | 0 | NO: 962 | 9 | NO: | | | |
| | | | | 1322 | | | |

TABLE 28
Dose-Response Data Table

| | | SEQ ID | | SEQ ID | | | |
|---|---|---|---|---|---|---|---|
| | Antisense | NO (AS − | Sense | NO (SS − | IC₅₀ | % Max | |
| Duplex ID | strand ID | mod) | strand ID | mod) | [pM] | Inhibition | Target |
| ETXM130 | ETXS260 | SEQ ID | ETXS259 | SEQ ID | 265 | 81 | HCII |
| | | NO: 617 | | NO: | | | |
| | | | | 977 | | | |
| ETXM132 | ETXS264 | SEQ ID | ETXS263 | SEQ ID | 142 | 89 | HCII |
| | | NO: 619 | | NO: | | | |
| | | | | 979 | | | |
| ETXM133 | ETXS266 | SEQ ID | ETXS265 | SEQ ID | 286 | 86 | HCII |
| | | NO: 620 | | NO: | | | |
| | | | | 980 | | | |
| ETXM145 | ETXS290 | SEQ ID | ETXS289 | SEQ ID | 131 | 82 | HCII |
| | | NO: 632 | | NO: | | | |
| | | | | 992 | | | |
| ETXM146 | ETXS292 | SEQ ID | ETXS291 | SEQ ID | 83 | 79 | HCII |
| | | NO: 633 | | NO: | | | |
| | | | | 993 | | | |
| ETXM156 | ETXS312 | SEQ ID | ETXS311 | SEQ ID | 918 | 90 | HCII |
| | | NO: 643 | | NO: | | | |
| | | | | 1003 | | | |
| ETXM160 | ETXS320 | SEQ ID | ETXS319 | SEQ ID | 583 | 81 | HCII |
| | | NO: 647 | | NO: | | | |
| | | | | 1007 | | | |
| ETXM180 | ETXS360 | SEQ ID | ETXS359 | SEQ ID | 78 | 87 | HCII |
| | | NO: 667 | | NO: | | | |
| | | | | 1027 | | | |
| ETXM182 | ETXS364 | SEQ ID | ETXS363 | SEQ ID | 65 | 81 | HCII |
| | | NO: 669 | | NO: | | | |
| | | | | 1029 | | | |
| ETXM188 | ETXS376 | SEQ ID | ETXS375 | SEQ ID | 519 | 88 | HCII |
| | | NO: 675 | | NO: | | | |
| | | | | 1035 | | | |
| ETXM253 | ETXS506 | SEQ ID | ETXS505 | SEQ ID | 294 | 82 | HCII |
| | | NO: 720 | | NO: | | | |
| | | | | 1080 | | | |
| ETXM258 | ETXS516 | SEQ ID | ETXS515 | SEQ ID | 212 | 89 | HCII |
| | | NO: 725 | | NO: | | | |
| | | | | 1085 | | | |
| ETXM272 | ETXS544 | SEQ ID | ETXS543 | SEQ ID | 348 | 84 | HCII |
| | | NO: 739 | | NO: | | | |
| | | | | 1099 | | | |
| ETXM273 | ETXS546 | SEQ ID | ETXS545 | SEQ ID | 320 | 86 | HCII |
| | | NO: 740 | | NO: | | | |
| | | | | 1100 | | | |
| ETXM293 | ETXS586 | SEQ ID | ETXS585 | SEQ ID | 213 | 73 | HCII |
| | | NO: 760 | | NO: | | | |
| | | | | 1120 | | | |
| ETXM309 | ETXS618 | SEQ ID | ETXS617 | SEQ ID | 253 | 72 | HCII |
| | | NO: 776 | | NO: | | | |
| | | | | 1136 | | | |
| ETXM313 | ETXS626 | SEQ ID | ETXS625 | SEQ ID | 327 | 84 | HCII |
| | | NO: 780 | | NO: | | | |
| | | | | 1140 | | | |
| ETXM314 | ETXS628 | SEQ ID | ETXS627 | SEQ ID | 214 | 81 | HCII |
| | | NO: 781 | | NO: | | | |
| | | | | 1141 | | | |
| ETXM320 | ETXS640 | SEQ ID | ETXS639 | SEQ ID | 556 | 77 | ZPI |
| | | NO: 787 | | NO: | | | |
| | | | | 1147 | | | |
| ETXM321 | ETXS642 | SEQ ID | ETXS641 | SEQ ID | 172 | 82 | ZPI |
| | | NO: 788 | | NO: | | | |
| | | | | 1148 | | | |
| ETXM322 | ETXS644 | SEQ ID | ETXS643 | SEQ ID | 101 | 79 | ZPI |
| | | NO: 789 | | NO: | | | |
| | | | | 1149 | | | |
| ETXM323 | ETXS646 | SEQ ID | ETXS645 | SEQ ID | 268 | 79 | ZPI |
| | | NO: 790 | | NO: | | | |
| | | | | 1150 | | | |
| ETXM325 | ETXS650 | SEQ ID | ETXS649 | SEQ ID | 274 | 79 | ZPI |
| | | NO: 792 | | NO: | | | |
| | | | | 1152 | | | |
| ETXM326 | ETXS652 | SEQ ID | ETXS651 | SEQ ID | 607 | 86 | ZPI |
| | | NO: 793 | | NO: | | | |
| | | | | 1153 | | | |
| ETXM332 | ETXS664 | SEQ ID | ETXS663 | SEQ ID | 81 | 85 | ZPI |
| | | NO: 799 | | NO: | | | |
| | | | | 1159 | | | |
| ETXM333 | ETXS666 | SEQ ID | ETXS665 | SEQ ID | 130 | 83 | ZPI |
| | | NO: 800 | | NO: | | | |
| | | | | 1160 | | | |
| ETXM338 | ETXS676 | SEQ ID | ETXS675 | SEQ ID | 33 | 77 | ZPI |
| | | NO: 805 | | NO: | | | |
| | | | | 1165 | | | |
| ETXM339 | ETXS678 | SEQ ID | ETXS677 | SEQ ID | 1180 | 78 | ZPI |
| | | NO: 806 | | NO: | | | |
| | | | | 1166 | | | |
| ETXM341 | ETXS682 | SEQ ID | ETXS681 | SEQ ID | 186 | 83 | ZPI |
| | | NO: 808 | | NO: | | | |
| | | | | 1168 | | | |
| ETXM342 | ETXS684 | SEQ ID | ETXS683 | SEQ ID | 71 | 63 | ZPI |
| | | NO: 809 | | NO: | | | |
| | | | | 1169 | | | |
| ETXM343 | ETXS686 | SEQ ID | ETXS685 | SEQ ID | 60 | 79 | ZPI |
| | | NO: 810 | | NO: | | | |
| | | | | 1170 | | | |
| ETXM452 | ETXS904 | SEQ ID | ETXS903 | SEQ ID | 82 | 78 | ZPI |
| | | NO: 899 | | NO: | | | |
| | | | | 1259 | | | |
| ETXM472 | ETXS944 | SEQ ID | ETXS943 | SEQ ID | 130 | 77 | ZPI |
| | | NO: 919 | | NO: | | | |
| | | | | 1279 | | | |
| ETXM492 | ETXS984 | SEQ ID | ETXS983 | SEQ ID | 47 | 84 | ZPI |
| | | NO: 939 | | NO: | | | |
| | | | | 1299 | | | |
| ETXM500 | ETXS1000 | SEQ ID | ETXS999 | SEQ ID | 607 | 70 | ZPI |
| | | NO: 947 | | NO: | | | |
| | | | | 1307 | | | |
| ETXM501 | ETXS1002 | SEQ ID | ETXS1001 | SEQ ID | 190 | 74 | ZPI |
| | | NO: 948 | | NO: | | | |
| | | | | 1308 | | | |
| ETXM502 | ETXS1004 | SEQ ID | ETXS1003 | SEQ ID | 132 | 76 | ZPI |
| | | NO: 949 | | NO: | | | |
| | | | | 1309 | | | |
| ETXM503 | ETXS1006 | SEQ ID | ETXS1005 | SEQ ID | 322 | 75 | ZPI |
| | | NO: 950 | | NO: | | | |
| | | | | 1310 | | | |
| ETXM505 | ETXS1010 | SEQ ID | ETXS1009 | SEQ ID | 199 | 72 | ZPI |
| | | NO: 952 | | NO: | | | |
| | | | | 1312 | | | |
| ETXM506 | ETXS1012 | SEQ ID | ETXS1011 | SEQ ID | 162 | 82 | ZPI |
| | | NO: 953 | | NO: | | | |
| | | | | 1313 | | | |
| ETXM512 | ETXS1024 | SEQ ID | ETXS1023 | SEQ ID | 76 | 84 | ZPI |
| | | NO: 959 | | NO: | | | |
| | | | | 1319 | | | |
| ETXM513 | ETXS1026 | SEQ ID | ETXS1025 | SEQ ID | 146 | 77 | ZPI |
| | | NO: 960 | | NO: | | | |
| | | | | 1320 | | | |

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

In case of ambiguity between the sequences in this specification and the sequences in the attached sequence listing, the sequences provided herein are considered to be the correct sequences.

1. A nucleic acid for inhibiting expression of a target gene, comprising a duplex region that comprises:
a first strand that is at least partially complementary to a portion of RNA transcribed from the target gene, and a second strand that is at least partially complementary to the first strand,
wherein the second strand comprises 2 consecutive abasic nucleosides in the 5′ terminal region of the second strand, wherein one such abasic nucleoside is a terminal nucleoside at the 5′ terminal region of the second strand and the other abasic nucleoside is a penultimate nucleoside at the 5′ terminal region of the second strand, wherein:
(a) said penultimate abasic nucleoside is connected to an adjacent first basic nucleoside in an adjacent 5′ near terminal region through a reversed internucleoside linkage;
(b) the reversed linkage is a 5-5′ reversed linkage; and
(c) the linkage between the terminal and penultimate abasic nucleosides is 3′-5′ when reading towards the terminus comprising the terminal and penultimate abasic nucleosides.

2. A nucleic acid for inhibiting expression of a target gene, comprising a duplex region that comprises:
a first strand that is at least partially complementary to a portion of RNA transcribed from the target gene, and a second strand that is at least partially complementary to the first strand,
wherein the second strand comprises 2 consecutive abasic nucleosides preferably in an overhang in the 3′ terminal region of the second strand, wherein one such abasic nucleoside is a terminal nucleoside at the 3′ terminal region of the second strand and the other abasic nucleoside is a penultimate nucleoside at the 3′ terminal region of the second strand, wherein:
(a) said penultimate abasic nucleoside is connected to an adjacent first basic nucleoside in an adjacent 3′ near terminal region through a reversed internucleoside linkage;
(b) the reversed linkage is a 3-3′ reversed linkage; and
(c) the linkage between the terminal and penultimate abasic nucleosides is 5′-3′ when reading towards the terminus comprising the terminal and penultimate abasic nucleosides.

3. A nucleic acid for inhibiting expression of a target gene, comprising a duplex region that comprises:
a first strand that is at least partially complementary to a portion of RNA transcribed from the target gene, and a second strand that is at least partially complementary to the first strand,
wherein:
(i) preferably the first strand and the second strand each has a length of 23 nucleosides;
(ii) the second strand comprises 2 consecutive abasic nucleosides in the 5′ terminal region of the second strand, wherein one such abasic nucleoside is a terminal nucleoside at the 5′ terminal region of the second strand and the other abasic nucleoside is a penultimate nucleoside at the 5′ terminal region of the second strand, wherein:
(a) said penultimate abasic nucleoside is connected to an adjacent first basic nucleoside in an adjacent 5′ near terminal region through a reversed internucleoside linkage; and
(b) the reversed linkage is a 5-5′ reversed linkage; and
(c) the linkage between the terminal and penultimate abasic nucleosides is 3-′5′ when reading towards the terminus comprising the terminal and penultimate abasic nucleosides;
(iii) two phosphorothioate internucleoside linkages are respectively present between three consecutive positions in said 5′ near terminal region of the second strand, wherein a first phosphorothioate internucleoside linkage is present between said first basic nucleoside of (a) and an adjacent second basic nucleoside in said 5′ near terminal region of the second strand, and a second phosphorothioate internucleoside linkage is present between said second basic nucleoside and an adjacent third basic nucleoside in said 5′ near terminal region of the second strand;
(iv) two phosphorothioate internucleoside linkages are respectively present between three consecutive positions in both 5′ and 3′ terminal regions of the first strand, whereby a terminal nucleoside respectively at each of the 5′ and 3′ terminal regions of said first strand is each attached to a respective 5′ and 3′ adjacent penultimate nucleoside by a phosphorothioate internucleoside linkage, and each 5′ and 3′ penultimate nucleoside is attached to a respective 5′ and 3′ adjacent antepenultimate nucleoside by a phosphorothioate internucleoside linkage; and
(v) the second strand of the nucleic acid is conjugated directly or indirectly to the one or more ligand moieties at the 3′ terminal region of the second strand.

4. A nucleic acid for inhibiting expression of a target gene, comprising a duplex region that comprises:
a first strand that is at least partially complementary to a portion of RNA transcribed from the target gene, and a second strand that is at least partially complementary to the first strand,
wherein:
(i) preferably the first strand and the second strand each has a length of 23 nucleosides;
(ii) the second strand comprises 2 consecutive abasic nucleosides in the 3′ terminal region of the second strand, wherein one such abasic nucleoside is a terminal nucleoside at the 3′ terminal region of the second strand and the other abasic nucleoside is a penultimate nucleoside at the 3′ terminal region of the second strand, wherein:
(a) said penultimate abasic nucleoside is connected to an adjacent first basic nucleoside in an adjacent 3′ near terminal region through a reversed internucleoside linkage; and
(b) the reversed linkage is a 3-3′ reversed linkage; and
(c) the linkage between the terminal and penultimate abasic nucleosides is 5-′3′ when reading towards the terminus comprising the terminal and penultimate abasic nucleosides;
(iii) two phosphorothioate internucleoside linkages are respectively present between three consecutive positions in said 3′ near terminal region of the second strand, wherein a first phosphorothioate internucleoside linkage is present between said first basic nucleoside of (a) and an adjacent second basic nucleoside in said 3′ near terminal region of the second strand, and a second phosphorothioate internucleoside linkage is present between said second basic nucleoside and an adjacent third basic nucleoside in said 3′ near terminal region of the second strand;
(iv) two phosphorothioate internucleoside linkages are respectively present between three consecutive positions in both 5′ and 3′ terminal regions of the first strand, whereby a terminal nucleoside respectively at each of the 5′ and 3′ terminal regions of said first strand is each attached to a respective 5′ and 3′ adjacent penultimate nucleoside by a phosphorothioate internucleoside linkage, and each 5′ and 3′ penultimate nucleoside is attached to a respective 5′ and 3′ adjacent antepenultimate nucleoside by a phosphorothioate internucleoside linkage; and
(v) the second strand of the nucleic acid is conjugated directly or indirectly to the one or more ligand moieties at the 5′ terminal region of the second strand.

5. A nucleic acid for inhibiting expression of a target gene, comprising a duplex region that comprises:
a first strand that is at least partially complementary to a portion of RNA transcribed from the target gene, and a second strand that is at least partially complementary to the first strand,
wherein the second strand comprises 2 consecutive abasic nucleosides in the 5′ terminal region of the second strand present as the following 5′ terminal motif 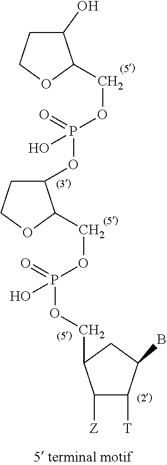
wherein:
B represents a nucleoside base,
T represents H, OH or a 2′ ribose modification,
Z represents the remaining nucleosides of said second strand.

6. A nucleic acid for inhibiting expression of a target gene, comprising a duplex region that comprises:
a first strand that is at least partially complementary to a portion of RNA transcribed from the target gene, and a second strand that is at least partially complementary to the first strand,
wherein the second strand comprises 2 consecutive abasic nucleosides in the 5′ terminal region of the second strand present as the following 5′ terminal motif 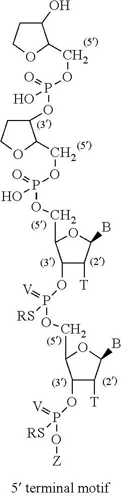
wherein:
B represents a nucleoside base,
T represents H, OH or a 2′ ribose modification,
V represents O or S (preferably O),
R represents H or C₁₋₄ alkyl (preferably H),
Z represents the remaining nucleosides of said second strand,
more preferably the following 5′ terminal motif 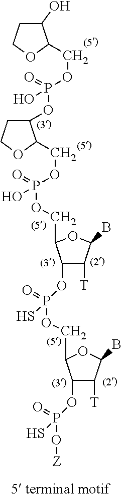
wherein:
B represents a nucleoside base,
T represents H, OH or a 2′ ribose modification,
Z represents the remaining nucleosides of said second strand.

7. A nucleic acid for inhibiting expression of a target gene, comprising a duplex region that comprises:
a first strand that is at least partially complementary to a portion of RNA transcribed from the target gene, and a second strand that is at least partially complementary to the first strand,
wherein the second strand comprises 2 consecutive abasic nucleosides in the 5′ terminal region of the second strand present as the following 5′ terminal motif 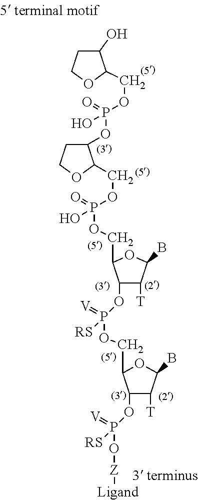
wherein:
B represents a nucleoside base,
T represents H, OH or a 2′ ribose modification,
V represents O or S (preferably O),
R represents H or C₁₋₄ alkyl (preferably H),
Z comprises 11 to 26 contiguous nucleosides, preferably 15 to 21 contiguous nucleosides, and more preferably 19 contiguous nucleosides,
more preferably the following 5′ terminal motif 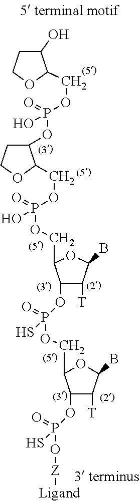
wherein:
B represents a nucleoside base,
T represents H, OH or a 2′ ribose modification,
Z comprises 11 to 26 contiguous nucleosides, preferably 15 to 21 contiguous nucleosides, and more preferably 19 contiguous nucleosides.

8. A nucleic acid for inhibiting expression of a target gene, comprising a duplex region that comprises:
a first strand that is at least partially complementary to a portion of RNA transcribed from the target gene, and a second strand that is at least partially complementary to the first strand,
wherein the second strand comprises 2 consecutive abasic nucleosides in the 3′ terminal region of the second strand present as the following 3′ terminal motif 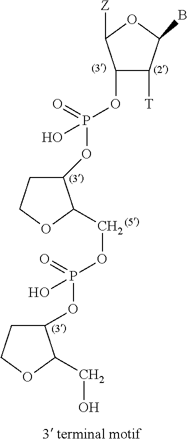
wherein:
B represents a nucleoside base,
T represents H, OH or a 2′ ribose modification,
Z represents the remaining nucleosides of said second strand.

9. A nucleic acid for inhibiting expression of a target gene, comprising a duplex region that comprises:
a first strand that is at least partially complementary to a portion of RNA transcribed from the target gene, and a second strand that is at least partially complementary to the first strand,
wherein the second strand comprises 2 consecutive abasic nucleosides in the 3′ terminal region of the second strand present as the following 3′ terminal motif 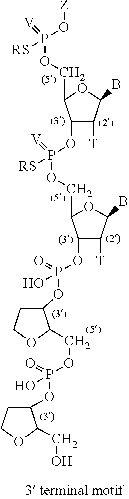
wherein:
B represents a nucleoside base,
T represents H, OH or a 2′ ribose modification,
V represents O or S (preferably O),
R represents H or C₁₋₄ alkyl (preferably H),
Z represents the remaining nucleosides of said second strand,
more preferably the following 3′ terminal motif 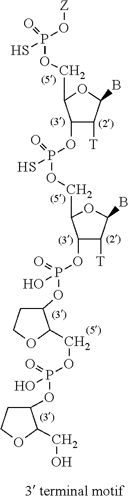
wherein:
B represents a nucleoside base,
T represents H, OH or a 2′ ribose modification,
Z represents the remaining nucleosides of said second strand.

10. A nucleic acid for inhibiting expression of a target gene, comprising a duplex region that comprises:
a first strand that is at least partially complementary to a portion of RNA transcribed from the target gene, and a second strand that is at least partially complementary to the first strand,
wherein the second strand comprises 2 consecutive abasic nucleosides in the 3′ terminal region of the second strand present as the following 3′ terminal motif 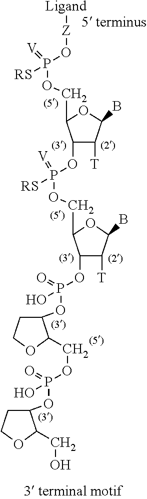
wherein:
B represents a nucleoside base,
T represents H, OH or a 2′ ribose modification,
V represents O or S (preferably O),
R represents H or C₁₋₄ alkyl (preferably H),
Z comprises 11 to 26 contiguous nucleosides, preferably 15 to 21 contiguous nucleosides, and more preferably 19 contiguous nucleosides,
more preferably the following 3′ terminal motif 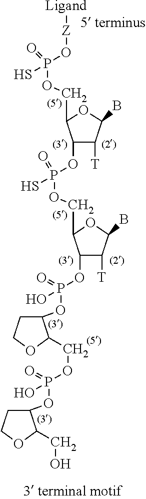
wherein:
B represents a nucleoside base,
T represents H, OH or a 2′ ribose modification,
Z comprises 11 to 26 contiguous nucleosides, preferably 15 to 21 contiguous nucleosides, and more preferably 19 contiguous nucleosides.

11. A nucleic acid according to any of claims 5 to 10, wherein the first strand comprises the following contiguous nucleosides represented by each N:
N(s)N(s)Nₓ(s)N(s)N
where X=11 to 26, preferably 15 to 21, and more preferably 19,
s represents a phosphorothioate internucleoside linkage.

12. A nucleic acid according to any preceding claim, wherein the nucleic acid is an siRNA oligonucleoside.

13. A nucleic acid according to any preceding claim, wherein one or more nucleosides of the first and/or second strand are modified, to form modified nucleosides.

14. A nucleic acid according to claim 13, wherein one or more nucleosides on the first and/or second strand comprise terminal modifications, base modifications, sugar modifications and/or backbone modifications.

15. A nucleic acid according to claim 13 or 14, wherein one or more nucleosides on the first and/or second strand comprise sugar modifications, wherein the modification is a modification at the 2′-OH group of the ribose sugar.

16. A nucleic acid according to claim 15, wherein the sugar modifications comprise 2′-Me and/or 2′-F modifications.

17. A nucleic acid according to claim 15, wherein the first strand comprises a 2′-F modification at any of position 2, position 6, position 14, or any combination thereof, counting from position 1 of said first strand.

18. A nucleic acid according to claim 15, wherein the second strand comprises a 2′-F modification at any of position 7, position 9, position 11, or any combination thereof, counting from position 1 of said second strand.

19. A nucleic acid according to claim 15, wherein the first and second strand each comprise 2′-Me and 2′-F modifications.

20. A nucleic according to any preceding claim, wherein the nucleic acid comprises at least one thermally destabilizing modification, suitably at one or more of positions 1 to 9 of the first strand counting from position 1 of the first strand, and/or at one or more of positions on the second strand aligned with positions 1 to 9 of the first strand, wherein the destabilizing modification is selected from a modified unlocked nucleic acid (IMUNA) and a glycol nucleic acid (GNA), preferably a glycol nucleic acid.

21. A nucleic acid according to claim 20, wherein the nucleic acid comprises at least one thermally destabilizing modification at position 7 of the first strand, counting from position 1 of the first strand.

22. A nucleic acid according to claim 15, which is an siRNA oligonucleoside, wherein the siRNA oligonucleoside comprises 3 or more 2′-F modifications at positions 6 to 12 of the second strand, such as 4, 5, 6 or 7 2′-F modifications at positions 6 to 12 of the second strand, counting from position 1 of said second strand.

23. A nucleic acid according to claim 15, which is an siRNA oligonucleoside, wherein said second strand comprises at least 3, such as 4, 5 or 6, 2′-Me modifications at positions 1 to 6 of the second strand, counting from position 1 of said second strand.

24. A nucleic acid according to claim 15, which is an siRNA oligonucleoside, wherein said first strand comprises at least 5 2′-Me consecutive modifications at the 3′ terminal region, preferably including the terminal nucleoside at the 3′ terminal region, or at least within 1 or 2 nucleosides from the terminal nucleoside at the 3′ terminal region.

25. A nucleic acid according to claim 15, which is an siRNA oligonucleoside, wherein said first strand comprises 7 2′-Me consecutive modifications at the 3′ terminal region, preferably including the terminal nucleoside at the 3′ terminal region.

26. A nucleic acid according to claim 15, wherein said modified nucleosides of said second strand comprise a modification pattern according to any one of the following (5′-3′):
ia-ia-Me-Me-Me-Me-Me-Me-F—F—F—F—F-Me-Me-Me-Me-Me-Me-Me-F-Me-Me, or
ia-ia-Me-Me-Me-Me-Me-F—F-Me-F—F—F—F-Me-Me-Me-Me-Me-Me-Me-Me-Me, or
ia-ia-Me-Me-Me-Me-Me-Me-F-Me-F—F—F—F-Me-Me-Me-Me-Me-Me-Me-Me-Me, or
ia-ia-Me-Me-Me-Me-Me-Me-Me-Me-F—F—F-Me-Me-Me-Me-Me-Me-Me-Me-Me-Me, or
ia-ia-Me-Me-Me-Me-Me-Me-F-Me-F—F—F-Me-Me-Me-Me-Me-Me-Me-Me-Me-Me, or
Me-Me-Me-Me-Me-Me-F—F—F—F—F-Me-Me-Me-Me-Me-Me-Me-F-Me-Me-ia-ia, or
Me-Me-Me-Me-Me-F—F-Me-F—F—F—F-Me-Me-Me-Me-Me-Me-Me-Me-Me-ia-ia, or
Me-Me-Me-Me-Me-Me-F-Me-F—F—F—F-Me-Me-Me-Me-Me-Me-Me-Me-Me-ia-ia, or
Me-Me-Me-Me-Me-Me-Me-Me-F—F—F-Me-Me-Me-Me-Me-Me-Me-Me-Me-Me-ia-ia, or
Me-Me-Me-Me-Me-Me-F-Me-F—F—F-Me-Me-Me-Me-Me-Me-Me-Me-Me-Me-ia-ia,
wherein is represents an inverted abasic nucleoside.

27. A nucleic acid according to claim 15, wherein said modified nucleosides comprise any of the following modification patterns:
Modification pattern 1:
Second strand (5′-3′): ia-ia-Me-Me-Me-Me-Me-Me-F—F—F—F—F-Me-Me-Me-Me-Me-Me-Me-F-Me-Me,
First strand (5′-3′): Me-F-Me-F-Me-F-Me-F—F-Me-Me-Me-Me-F-Me-F-Me-Me-Me-Me-Me-Me-Me
Or Modification pattern 2:
Second strand (5′-3′): ia-ia-Me-Me-Me-Me-Me-F—F-Me-F—F—F—F-Me-Me-Me-Me-Me-Me-Me-Me-Me,
First strand (5′-3′): Me-F-Me-F-Me-F-Me-F—F-Me-Me-Me-Me-F-Me-F-Me-Me-Me-Me-Me-Me-Me
Or Modification pattern 3:
Second strand (5′-3′): ia-ia-Me-Me-Me-Me-Me-Me-F-Me-F—F—F—F-Me-Me-Me-Me-Me-Me-Me-Me-Me,
First strand (5′-3′): Me-F-Me-F-Me-F-Me-F—F-Me-Me-Me-Me-F-Me-F-Me-Me-Me-Me-Me-Me-Me
Or Modification pattern 4:
Second strand (5′-3′): ia-ia-Me-Me-Me-Me-Me-Me-F-Me-F—F—F—F-Me-Me-Me-Me-Me-Me-Me-Me-Me,
First strand (5′-3′): Me-F-Me-F-Me-F-Me-Me-Me-Me-Me-Me-Me-F-Me-F-Me-Me-Me-Me-Me-Me-Me
Or Modification pattern 5:
Second strand (5′-3′): ia-ia-Me-Me-Me-Me-Me-Me-Me-Me-F—F—F-Me-Me-Me-Me-Me-Me-Me-Me-Me-Me,
First strand (5′-3′): Me-F-Me-Me-Me-F-Me-Me-F-Me-Me-Me-Me-F-Me-F-Me-Me-Me-Me-Me-Me-Me
Or Modification pattern 6:
Second strand (5′-3′): ia-ia-Me-Me-Me-Me-Me-Me-F-Me-F—F—F-Me-Me-Me-Me-Me-Me-Me-Me-Me-Me,
First strand (5′-3′): Me-F-Me-Me-Me-F-Me-Me-F-Me-Me-Me-Me-F-Me-F-Me-Me-Me-Me-Me-Me-Me
Or Modification pattern 7:
Second strand (5′-3′): Me-Me-Me-Me-Me-Me-F—F—F—F—F-Me-Me-Me-Me-Me-Me-Me-F-Me-Me-ia-ia,
First strand (5′-3′): Me-F-Me-F-Me-F-Me-F—F-Me-Me-Me-Me-F-Me-F-Me-Me-Me-Me-Me-Me-Me
Or Modification pattern 8:
Second strand (5′-3′): Me-Me-Me-Me-Me-F—F-Me-F—F—F—F-Me-Me-Me-Me-Me-Me-Me-Me-Me-ia-ia,
First strand (5′-3′): Me-F-Me-F-Me-F-Me-F—F-Me-Me-Me-Me-F-Me-F-Me-Me-Me-Me-Me-Me-Me
Or Modification pattern 9:
Second strand (5′-3′): Me-Me-Me-Me-Me-Me-F-Me-F—F—F—F-Me-Me-Me-Me-Me-Me-Me-Me-Me-ia-ia,
First strand (5′-3′): Me-F-Me-F-Me-F-Me-F—F-Me-Me-Me-Me-F-Me-F-Me-Me-Me-Me-Me-Me-Me
Or Modification pattern 10:
Second strand (5′-3′): Me-Me-Me-Me-Me-Me-F-Me-F—F—F—F-Me-Me-Me-Me-Me-Me-Me-Me-Me-ia-ia,
First strand (5′-3′): Me-F-Me-F-Me-F-Me-Me-Me-Me-Me-Me-Me-F-Me-F-Me-Me-Me-Me-Me-Me-Me
Or Modification pattern 11:
Second strand (5′-3′): Me-Me-Me-Me-Me-Me-Me-Me-F—F—F-Me-Me-Me-Me-Me-Me-Me-Me-Me-Me-ia-ia,
First strand (5′-3′): Me-F-Me-Me-Me-F-Me-Me-F-Me-Me-Me-Me-F-Me-F-Me-Me-Me-Me-Me-Me-Me
Or Modification pattern 12:
Second strand (5′-3′): Me-Me-Me-Me-Me-Me-F-Me-F—F—F-Me-Me-Me-Me-Me-Me-Me-Me-Me-Me-ia-ia,
First strand (5′-3′): Me-F-Me-Me-Me-F-Me-Me-F-Me-Me-Me-Me-F-Me-F-Me-Me-Me-Me-Me-Me-Me

28. A nucleic acid according to any preceding claim, wherein said first strand comprises at least 17 contiguous nucleosides differing by 0 or 1 nucleosides from any one of the first strand sequences as listed in Table 23.

29. A nucleic acid according to any preceding claim, wherein said first strand comprises at least 17 contiguous nucleosides differing by 0 or 1 nucleosides from any one of first strand modified sequences as listed in Table 24.

30. A nucleic acid according to claim 28 or 29, wherein the first strand comprises nucleosides 2-18 of any one of the sequences defined in claim 28 or 29.

31. A nucleic acid according to any preceding claim, wherein the second strand comprises a nucleoside sequence of at least 17 contiguous nucleosides differing by 0 or 1 nucleosides from any one of second strand sequences as listed in Table 23, and wherein the second strand has a region of at least 85% complementarity over the 17 contiguous nucleosides to the first strand.

32. A nucleic acid according to any preceding claim, wherein the second strand comprises a nucleoside sequence of at least 17 contiguous nucleosides differing by 0 or 1 nucleosides from any one of second strand modified sequences as listed in Table 25, and wherein the second strand has a region of at least 85% complementarity over the 17 contiguous nucleosides to the first strand.

33. A nucleic acid according to any preceding claim, wherein the first strand comprises any one of the first strand sequences as listed in Table 23.

34. A nucleic acid according to any preceding claim, wherein the first strand comprises any one of first strand modified sequences as listed in Table 24.

35. A nucleic acid according to any preceding claim, wherein the second strand comprises any one of second strand sequences as listed in Table 23.

36. A nucleic acid according to any preceding claim, wherein the second strand comprises any one of second strand modified sequences as listed in Table 25.

37. A nucleic acid according to any preceding claim, wherein the nucleic acid is conjugated directly or indirectly to one or more ligand moieties, optionally wherein said ligand moiety is present at a terminal region of the second strand, preferably at the 3′ terminal region thereof.

38. A nucleic acid according to claim 37, wherein the ligand moiety comprises:
(i) one or more N-acetyl galactosamine (GalNAc) ligands, and/or
(ii) one or more N-acetyl galactosamine (GalNAc) ligand derivatives, and/or
(iii) one or more N-acetyl galactosamine (GalNAc) ligands and/or derivatives thereof, conjugated to the nucleic acid through a linker.

39. A nucleic acid according to claim 38, wherein said one or more GalNAc ligands and/or GalNAc ligand derivatives are conjugated directly or indirectly to the 5′ or 3′ terminal region of the second strand of the nucleic acid, preferably at the 3′ terminal region thereof.

40. A nucleic acid according to any one of claims 37 to 39, wherein the ligand moiety comprises the following structure:

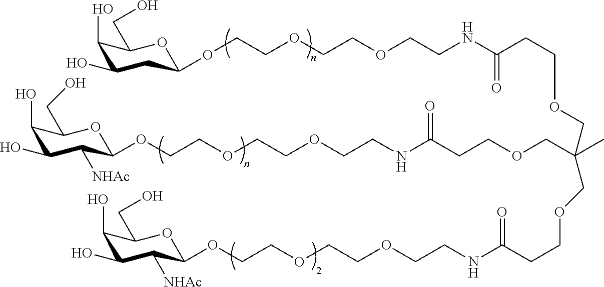

41. A nucleic acid according to any one of claims 37 to 40, having the structure: 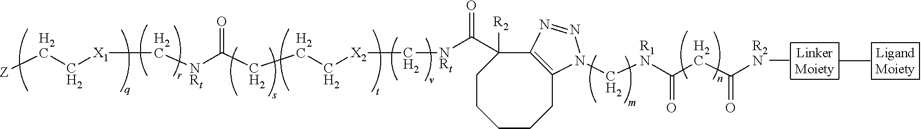
wherein:
R₁ at each occurrence is independently selected from the group consisting of hydrogen, methyl and ethyl;
R₂ is selected from the group consisting of hydrogen, hydroxy, —OC₁₋₃alkyl, —C(═O)OC₁₋₃alkyl, halo and nitro;
X₁ and X₂ at each occurrence are independently selected from the group consisting of methylene, oxygen and sulfur;
m is an integer of from 1 to 6;
n is an integer of from 1 to 10;
q, r, s, t, v are independently integers from 0 to 4, with the proviso that:
(i) q and r cannot both be 0 at the same time; and
(ii) s, t and v cannot all be 0 at the same time;
Z is an oligonucleoside.

42. A nucleic acid according to any one of claims 37 to 40, having the structure: 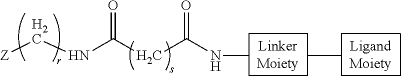
wherein:
r and s are independently an integer selected from 1 to 16; and
Z is an oligonucleoside.

43. A pharmaceutical composition comprising a nucleic acid according to any preceding claim, in combination with a pharmaceutically acceptable excipient or carrier.

44. A nucleic acid or pharmaceutical composition according to any preceding claim, for use in therapy.

45. A nucleic acid or pharmaceutical composition according to any preceding claim, for use in prevention or treatment of a disease related to a disorder of haemostasis, such as a disease related to a disorder of haemostasis, such as haemophilia.

46. A nucleic acid or pharmaceutical composition according to any preceding claim, for use in prevention or treatment of diabetes.
